(19) United States
(12) Patent Application Publication
(10) Pub. No.: US 2023/0055237 A1
(43) Pub. Date: Feb. 23, 2023

(54) COMPOUNDS AND METHODS FOR THE TREATMENT OF CYSTIC FIBROSIS

(71) Applicant: Flatley Discovery Lab, LLC, Canton, MA (US)

(72) Inventors: Michael P. Zawistoski, West Warwick, RI (US); Christopher Oalmann, Watertown, MA (US); Feng Li, Winchester, MA (US); Andrew Kolodziej, Winchester, MA (US); Marshall Morningstar, Framingham, MA (US)

(21) Appl. No.: 17/383,797

(22) Filed: Jul. 23, 2021

Related U.S. Application Data
(63) Continuation of application No. PCT/US2020/015441, filed on Jan. 28, 2020.
(60) Provisional application No. 62/797,743, filed on Jan. 28, 2019.
(60) Provisional application No. 62/931,502, filed on Nov. 6, 2019.

(51) Int. Cl. C07D 498/20; C07D 487/20; C07D 487/10; C07D 519/00; A61K 45/06

(52) U.S. Cl. CPC C07D 498/20; C07D 487/20; A61K 45/06; C07D 519/00; C07D 487/10

(57) ABSTRACT

The invention relates to a compound of Formula I, pharmaceutical compositions comprising a compound of Formula I,

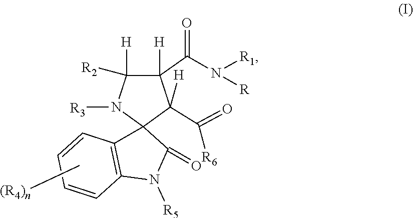

and pharmaceutically acceptable slats thereof, pharmaceutical compositions comprising such compounds and methods of treating cystic fibrosis comprising the step of administering a therapeutically effective amount of a compound of Formula Ito a subject in need thereof.

### RELATED APPLICATIONS

This application is a continuation of International Application No. PCT/US2020/015441, which designated the United States and was filed on Jan. 28, 2020, published in English, which claims the benefit of U.S. Provisional Application No. 62/797,743, filed on Jan. 28, 2019 and U.S. Provisional Application No. 62/931,502, filed on Nov. 6, 2019. The entire teachings of the above applications are incorporated herein by reference.

### BACKGROUND

Cystic fibrosis (CF) is a lethal, recessive, genetic disease affecting approximately 1 in 2500 live births among Caucasians. (Cohen-Cymberknoh, M. et al., Am. J. Respir. Crit. Care Med. 1463-1471, 2011; Boat et al., The Metabolic Basis of Inherited Disease, 6th ed., pp 2649-2680, McGraw Hill, NY (1989)). Approximately 1 in 25 persons are carriers of the disease. The major symptoms of cystic fibrosis include chronic pulmonary disease, pancreatic exocrine insufficiency, and elevated sweat electrolyte levels. The symptoms are consistent with cystic fibrosis being an exocrine disorder. (Hantash F: U.S. Patent Application No. 20060057593).

The CF gene codes for a cAMP/PKA-dependent, ATP-requiring, membrane chloride ion channel, generally found in the apical membranes of many secreting epithelia and is known as CFTR (cystic fibrosis transmembrane conductance regulator). There are currently over 1900 known mutations affecting CFTR, many of which give rise to a disease phenotype. Around 75% of CF alleles contain the ΔF508 mutation in which a triplet codon has been lost, leading to a missing phenylalanine at position 508 in the protein. This altered protein fails to be trafficked to the correct location in the cell and is generally destroyed by the proteasome. The small amount that does reach the correct location functions poorly. (Cuthbert A W, British Journal of Pharmacology, 163(1), 173-183, 2011).

Mutations in the CFTR gene result in absence or dysfunction of the protein that regulates ion transport across the apical membrane at the surface of certain epithelia. Although CFTR functions mainly as a chloride channel, it has many other roles, including inhibition of sodium transport through the epithelial sodium channel, regulation of the outwardly rectifying chloride channel, ATP channels, intracellular vesicle transport, and inhibition of endogenous calcium-activated chloride channels. CFTR is also involved in bicarbonate-chloride exchange. A deficiency in bicarbonate secretion leads to poor solubility and aggregation of luminal mucins. Obstruction of intrapancreatic ducts with thickened secretions causes autolysis of pancreatic tissue with replacement of the body of the pancreas with fat, leading to pancreatic insufficiency with subsequent malnutrition. In the lungs, CFTR dysfunction leads to airway surface liquid (ASL) depletion and thickened and viscous mucus that adheres to airway surfaces. The result is decreased mucociliary clearance (MCC) and impaired host defenses. Dehydrated, thickened secretions lead to endobronchial infection with a limited spectrum of distinctive bacteria, mainly Staphylococcus aureus and Pseudomonas aeruginosa, and an exaggerated inflammatory response leading to development of bronchiectasis and progressive obstructive airways disease. Pulmonary insufficiency is responsible for most CF-related deaths. (Cohen-Cymberknoh, M. et al., Am. J. Respir. Crit. Care Med. 1463-1471, 2011).

The prognosis for the treatment of CF has improved over the last 40 years. This was achieved by improving pancreatic enzyme supplements, drugs designed to treat pulmonary infection, reduce inflammation and enhance mucociliary clearance. Currently the therapeutic challenges are to correct the biochemical defect of CF and to identify effective treatments for chronic respiratory infection. (Frerichs C. et al., Expert Opin Pharmacother. 10(7), 1191-202, 2009).

### SUMMARY OF THE INVENTION

In one embodiment, the invention relates to a compound of Formula (I)

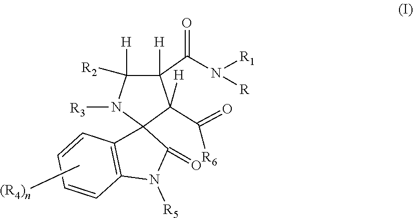

or a pharmaceutically acceptable salt thereof, wherein:
R and R₁ are independently selected from hydrogen, optionally substituted alkyl; optionally substituted alkenyl, optionally substituted alkynyl, optionally substituted cycloalkyl, optionally substituted aryl, optionally substituted arylalkyl, optionally substituted heteroaryl or optionally substituted heteroarylalkyl;
   or R and R₁, together with the nitrogen atom to which they are attached, form an optionally substituted 3 to 7-membered heterocyclyl;
R₂ is hydrogen, optionally substituted alkyl, optionally substituted aryl, optionally substituted arylalkyl, optionally substituted heteroaryl or optionally substituted heteroarylalkyl; in certain embodiments, R₂ is hydrogen, optionally substituted alkyl, optionally substituted aryl or optionally substituted arylalkyl;
R₃ is hydrogen, optionally substituted alkyl, R₇C(O)—, R₇SO₂- or R₇NHC(O)—; or R₂ and R₃, together with the atoms to which they are attached, form an optionally substituted 3 to 7-membered heterocyclyl;
   Each R₄ is independently halogen, optionally substituted alkyl, CN, optionally substituted alkoxy, NRi2R₁₃, or hydroxy;
R₃ is hydrogen, optionally substituted alkyl, optionally substituted alkenyl, optionally substituted alkynyl or optionally substituted cycloalkyl;
R₆ is OR₈ or NR₉R₁₀; or R₆ is —SR₈;
R₇ is optionally substituted alkyl, optionally substituted cycloalkyl, optionally substituted aryl, or optionally substituted arylalkyl;
R₈ is hydrogen, optionally substituted alkyl; optionally substituted alkenyl, optionally substituted alkynyl, optionally substituted cycloalkyl, optionally substituted aryl or optionally substituted heteroaryl;
R₉ is hydrogen, OR₁₁, optionally substituted alkyl, optionally substituted alkenyl, optionally substituted alkynyl, optionally substituted cycloalkyl; optionally substituted aryl or optionally substituted heteroaryl; or R₉ is optionally substituted heterocyclyl, SO₂R₈, SO₂NRₐR_{b} or N(Rₐ)R_{b};
Rₐ and R_{b} are each independently hydrogen, optionally substituted alkyl; optionally substituted alkenyl, optionally substituted alkynyl, optionally substituted cycloalkyl, optionally substituted aryl or optionally substituted heteroaryl;
R₁₀ is hydrogen; optionally substituted alkyl, optionally substituted alkenyl, optionally substituted alkynyl, optionally substituted cycloalkyl; optionally substituted aryl or optionally substituted heteroaryl;
   or R₉ and R₁₀, together with the nitrogen atom to which they are attached, form an optionally substituted heterocyclyl;
R₁₁ is hydrogen or optionally substituted alkyl;
R₁₂ and R₁₃ are each independently hydrogen, optionally substituted alkyl, R₇C(O)—, R₇SO₂— or R₇NHC(O)—;
   or R₁₂ and R₁₃, together with the nitrogen atom to which they are attached, form an optionally substituted heterocyclyl; and
   n is 0, 1, 2, 3 or 4; preferably n is 1 or 2.

In another embodiment, the present invention relates to a pharmaceutical composition comprising a compound of Formula (I), or a pharmaceutically acceptable salt thereof, and a pharmaceutically acceptable carrier or excipient.

In another embodiment, the present invention relates to a method of treating a CFTR-mediated disease or disorder, such as cystic fibrosis, in a subject in need thereof, comprising administering to the subject a therapeutically effective amount of a compound of Formula (I) or a pharmaceutically acceptable salt thereof.

### DETAILED DESCRIPTION OF THE INVENTION

The present invention relates to compounds of Formula (I) and pharmaceutically salts thereof, pharmaceutical compositions comprising such compounds and methods of using such compounds for treating a CFTR-mediated disease or condition in a subject in need thereof.

In certain embodiments, the compounds of the invention have the absolute stereochemistry shown in Formula (Ia) or Formula (Ib).

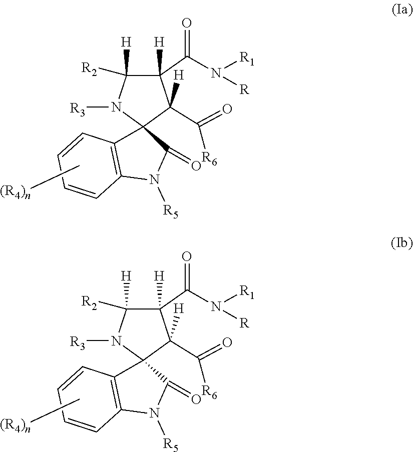

In certain embodiments of the compounds of the invention, R₁ is optionally substituted aryl, optionally substituted heteroaryl, optionally substituted arylalkyl, such as optionally substituted aryl-C₁-C₆-alkyl or optionally substituted heteroarylalkyl, such as heteroaryl-C₁-C₆-alkyl; preferably optionally substituted phenyl or optionally substituted 6-membered heteroaryl.

In certain embodiments of the compounds of the invention, R is hydrogen, optionally substituted C₁-C₆-alkyl; optionally substituted C₃-C₈-cycloalkyl; in certain embodiments, R is hydrogen, methyl, ethyl, n-propyl, isopropyl, n-butyl, t-butyl, sec-butyl, n-pentyl, neopentyl, optionally substituted C₃-C₆-cycloalkyl, optionally substituted C₃-C₆-cycloalkylmethyl, 2-dimethylaminoethyl, or 3-hydroxycyclobutyl. In certain embodiments, R is optionally substituted C₃-C₁₂-cycloalkyl-C₁-C₆-alkyl, preferably optionally substituted C₃-C₁₂-cycloalkyl-methyl. In certain embodiments, R is hydrogen or C₁-C₆-alkyl, such as hydrogen or methyl. In certain embodiments, R is a branched C₃-C₁₀-alkyl, preferably a branched C₃-C₈-alkyl. In certain embodiments, R is a (β-branched C₄-C₁₀-alkyl, such as 2,2,3,3,-tetramethylbutyl or 2,2,-dimethylpropyl.

In certain embodiments of the compounds of the invention, R₂ is hydrogen, optionally substituted C₁-C₆-alkyl, optionally substituted aryl-C₁-C₆-alkyl, or optionally substituted heteroaryl-C₁-C₆-alkyl. In certain embodiments, R₂ is hydrogen, C₁-C₄-alkyl, halo-C₁-C₄-alkyl, optionally substituted arylmethyl, or optionally substituted heteroarylmethyl. In certain embodiments, R₂ is hydrogen, benzyl, optionally substituted phenyl-CF₂—, optionally substituted heteroaryl-CF₂—, benzyl-O—CH₂—, CF₃, CF₃CH₂— or isopropyl. In certain embodiments, R₂ is hydrogen, C₁-C₄-alkyl, halo-C₁-C₄-alkyl, aryl optionally substituted with 1 to 5 halogen or aryl-C₁-C₂-alkyl optionally substituted with 1 to 5 halogen. In certain embodiments, R₂ is hydrogen, CF₃, isopropyl, benzyl, benzyl-O—CH₂—, 3-hydroxy-n-propyl, or α,α-difluorobenzyl.

In certain embodiments of the compounds of the invention, R₃ is hydrogen, C₁-C₄-alkyl, halo-C₁-C₄-alkyl, C₁-C₄-alkylC(O)—, aryl-C₁-C₄-alkylC(O)—, aryl-C₁-C₄-alkyl S(O)2-, aryl-C₁-C₄-alkylNHC(O)—, or arylNHC(O)—. In certain embodiments, R₃ is hydrogen, methyl, CF₃CH₂—, acetyl, propionyl, phenethylC(O)—, phenethylSO₂—, benzylNHC(O)— or phenylNHC(O)—.

In certain embodiments of the compounds of the invention, at least one of R₂ and R₃ is hydrogen.

In certain embodiments, R₂ and R₃, together with the atoms to which they are attached, form an optionally substituted saturated 4 to 6-membered heterocyclyl, preferably an optionally substituted saturated 5-membered heterocyclyl, and more preferably an optionally substituted pyrollidine. In certain embodiments, R₂ and R₃, together with the atoms to which they are attached, form an optionally substituted saturated 6-membered heterocyclyl, such as an optionally substituted piperidinyl or optionally substituted morpholyl. In certain embodiments, the saturated 4 to 6-membered heterocyclyl is unsubstituted or substituted with one or more substituents independently selected from halogen, CN, hydroxyl, C₁-C₃-alkoxy, halo-C₁-C₃-alkoxy, C₁-C₃-alkyl, halo-C₁-C₃-alkyl, a spiro cycloalkyl, a spiro heterocyclyl or an optionally substituted C₁-C₃-alkylidene.

In certain embodiments of the compouns of the invention, each R₄ is independently halo, such as chloro or fluoro.

In certain embodiments of the compounds of the invention, R₅ is hydrogen or C₁-C₆-alkyl; preferably hydrogen or methyl;

In certain embodiments of the compounds of the invention, R₆ is OR₈, and R₅ is hydrogen, optionally substituted C₁-C₁₀-alkyl or optionally substituted C₂-C₁₀-alkenyl. In certain embodiments, R₈ is hydrogen or optionally substituted C₁-C₁₀-alkyl. In certain embodiments, R₈ is hydrogen, C₁-C₄-alkyl or allyl. In certain embodiments, R₈ is —CH₂-O—R_{c}, where R_{c} is —C(O)—C₁-C₈-alkyl or

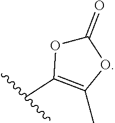 [SMILES: *c1oc(=O)oc1C]

In certain embodiments of the compounds of the invention, R₆ is NR₉R₁₀. In certain embodiments, R₉ and R₁₀ are both C₁-C₄-alkyl, preferably methyl. In certain embodiments, R₉ is OH or O—C₁-C₂-alkyl, preferably methyl and R₁₀ is hydrogen or C₁-C₃-alkyl, preferably hydrogen or methyl. In certain embodiments, R₉ is SO₂R₈ or SO₂NRₐRb. In certain embodiments, R₉ is —SO₂-C₁-C₄-alkyl, —SO₂-phenyl, —SO₂NH2 or —SO₂N(CH₃)₂.

In certain embodiments of the compounds of the invention, R₁ is optionally substituted aryl or heteroaryl, preferably optionally substituted phenyl or optionally substituted 6-membered heteroaryl; R is hydrogen, C₁-C₈-alkyl or C₁-C₆-alkyl; preferably hydrogen,methyl or a β-branched C₄-C₁₀-alkyl; R₅ is hydrogen or C₁-C₆-alkyl; preferably hydrogen or methyl; and R₆ is OR₈, and R₈ is hydrogen, or optionally substituted C₁-C₁₀-alkyl; or R₈ is hydrogen, optionally substituted C₁-C₁₀-alkyl; or optionally substituted C₂-C₆-alkenyl.

In certain embodiments, the compound of Formula (I) is represented by Formula (II),

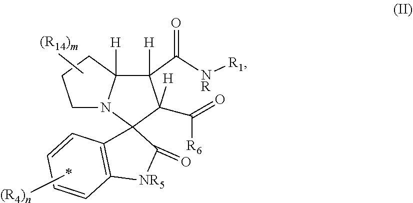

wherein m is 0, 1, 2, 3, 4, 5 or 6; and
each R₁₄ is independently hydroxyl, protected hydroxyl, cyano, amino, protected amino, halogen, optionally substituted alkoxy, or optionally substituted alkyl; or
two adjacent R₁₄ groups, together with the carbon atoms to which they are attached, form an optionally substituted fused 3 to 7-membered carbocylyl or heterocyclyl; or
two geminal R₁₄ groups, together with the carbon atom to which they are attached, form an optionally substituted spiro 3 to 7-membered carbocyclyl or heterocyclyl; or
two geminal R₁₄ groups together form (R₁₅)₂C=, wherein each R₁₅ is independently hydrogen, halogen, C₁-C₄-alkyl or halo-C₁-C₄-alkyl. In certain embodiments of the compounds of Formula II, m is 0 or 2. In certain embodiments, m is 2 and both R₁₄ groups are attached to the same carbon atom.

In certain embodiments, the compounds of Formula (II) have the absolute stereochemistry shown in Formula (IIa) or Formula (IIb).

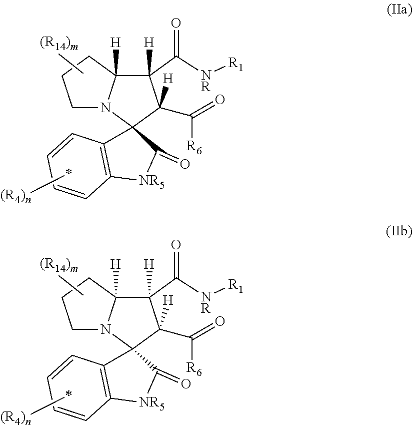

In certain embodiments, the compound of Formula I is represented by Formula (III),

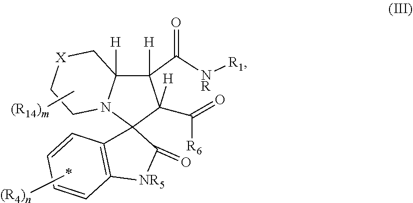

wherein X is O or C(Rₐ)₂, and each Rₐ is independently hydrogen, hydroxyl, protected hydroxyl, cyano, amino, protected amino, halogen, optionally substituted alkoxy, or optionally substituted alkyl.

In certain embodiments, the compounds of Formula (III) have the absolute stereochemistry shown in Formula (IIIa) or Formula (IIIb).

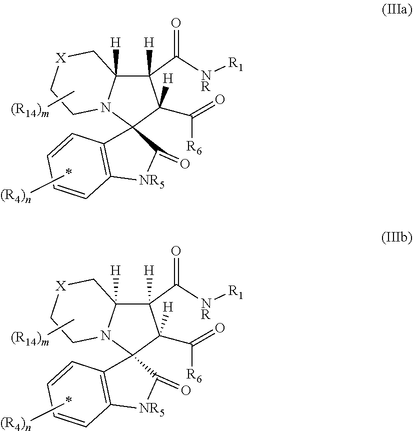

In certain embodiments, the compound of Formula I is represented by Formula (IV),

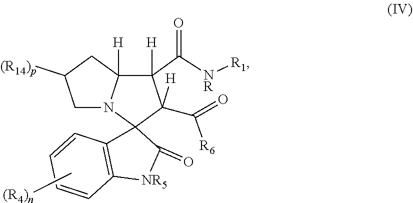

or a pharmaceutically acceptable salt thereof, wherein R₁₄ is as previously defined and p is 0, 1 or 2.

In certain embodiments, the compounds of Formula (IV) have the absolute stereochemistry shown in Formula (IVa) or Formula (IVb).

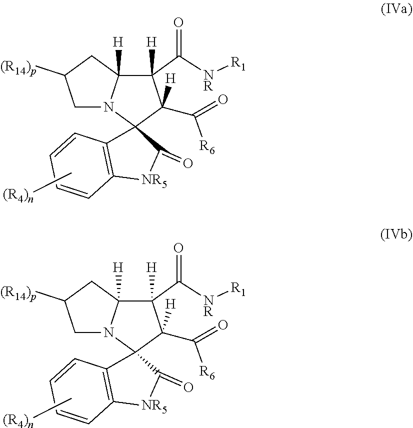

In certain embodiments, the compound of Formula I is represented by Formula (V),

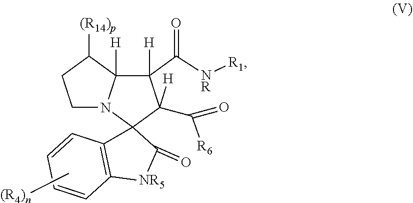

or a pharmaceutically acceptable salt thereof, wherein R₁₄ and p are as previously defined.

In certain embodiments, the compounds of Formula (V) have the absolute stereochemistry shown in Formula (Va) or Formula (Vb).

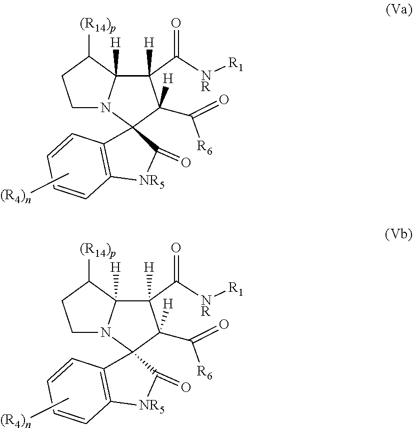

In certain embodiments of the compounds of the invention, R₁ is optionally substituted aryl, or optionally substituted 5- or 6-membered heteroaryl, for example, phenyl, naphthyl, pyridyl, pyrazinyl, pyrimidyl, pyrazolyl, oxazolyl, oxadiazolyl, thiazolyl, thiadiazolyl, triazolyl or pyrrolyl. In certain embodiments, R₁ is optionally substituted fused bicyclic heteroaryl, for example, quinolyl, quinazolyl, naphthyl, benzimidazolyl, isoquinolyl, pyrazopyridyl, benzothiazolyl, naphthyridyl, indolyl, or indazolyl. In certain embodiments, R₁ is optionally substituted phenyl-C₁-C₆-alkyl, optionally substituted heteroaryl-C₁-C₆-alkyl, or an optionally substituted biaryl group, such as optionally substituted biphenyl, phenylheteroaryl or heteroarylphenyl, including phenylpyrazyl.

Preferably, R₁ is unsubstituted or substituted with 1, 2 or 3 substituents independently selected from C₁-C₄-alkyl, halo-C₁-C₄-alkyl, halogen, C₁-C₄-alkoxy and halo-C₁-C₄-alkoxy. More preferably, the substituents are independently selected from methyl, methoxy, fluoro, chloro, methoxy, CHF2, CF₃, CHF2O— and CF₃₀—.

In certain embodiments, R₁ is selected from the groups below.

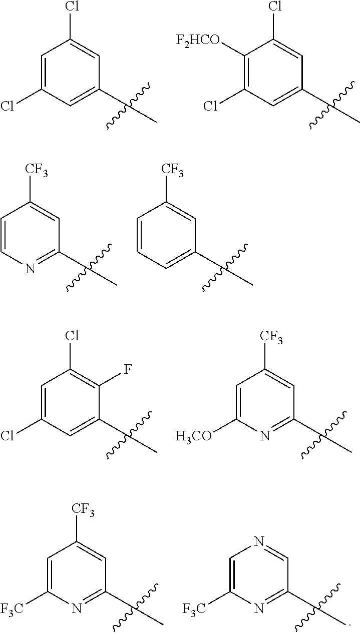

In certain embodiments, R₁ is represented by

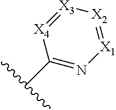

where X₁-X₄ are each independently N or CR₁₇, where each R₁₇ is independently hydrogen, optionally substituted alkyl, optionally substituted alkoxy or halogen. In certain embodiments, each R₁₇ is independently H, CF₃, CH₃, OCH₃, OCF₃ or halogen. Preferably no more than two of X₁, X₂, X₃ and X₄ are N. More preferably, no more than one of X₁, X₂, X₃ and X₄ is N.

In certain embodiments, R₁ is selected from the groups shown below:

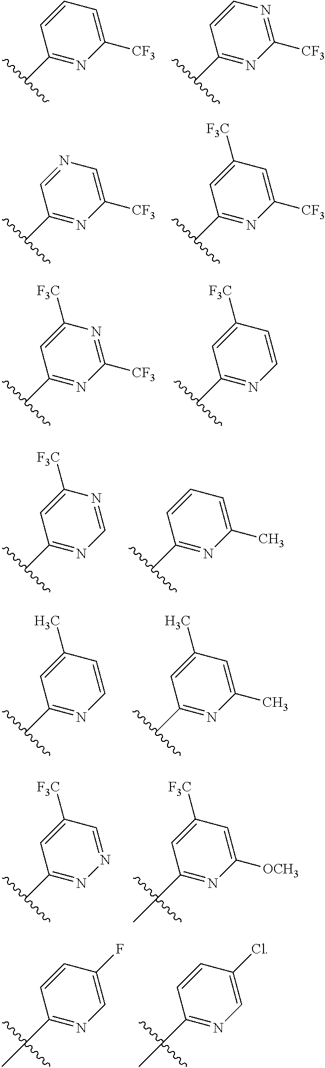

In certain embodiments of the compounds of the invention, R₁ is represented by

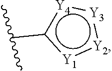

where one of Y₁, Y₂, Y₃ and Y₄ is O, S or NR₁₆, and the remainder are independently N or CR₁₇, where R₁₆ is hydrogen, optionally substituted alkyl, R₇C(O)—, R₇SO₂— or R₇NHC(O)— and R₁₇ is hydrogen, optionally substituted alkyl, optionally substituted alkoxy, CN or halogen. Preferably R₁₆ is hydrogen or methyl. Preferably R₁₇ is H; CF₃; CN; C₁-C₄-alkyl, such as CH₃; OCH₃; OCF₃ or halogen. Preferably at least one of Y₁ to Y₄ is CR₁₇. In certain embodiments, Y₃ is C—CF₃, one of Y₁, Y₂ and Y₄ is O, S or NR₁₆, and the remainder are independently N or CR₁₇. In certain embodiments, R₁ is selected from the groups shown below:

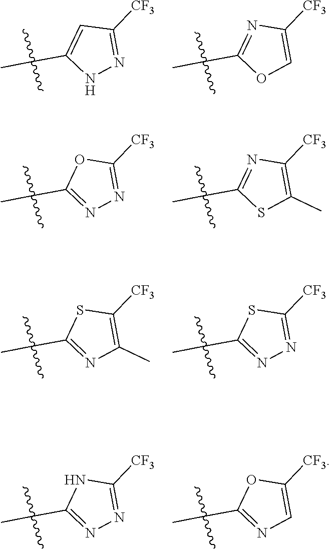

In certain embodiments of the compounds of Formula II,

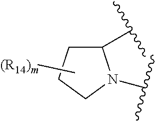

is selected from the groups shown below:

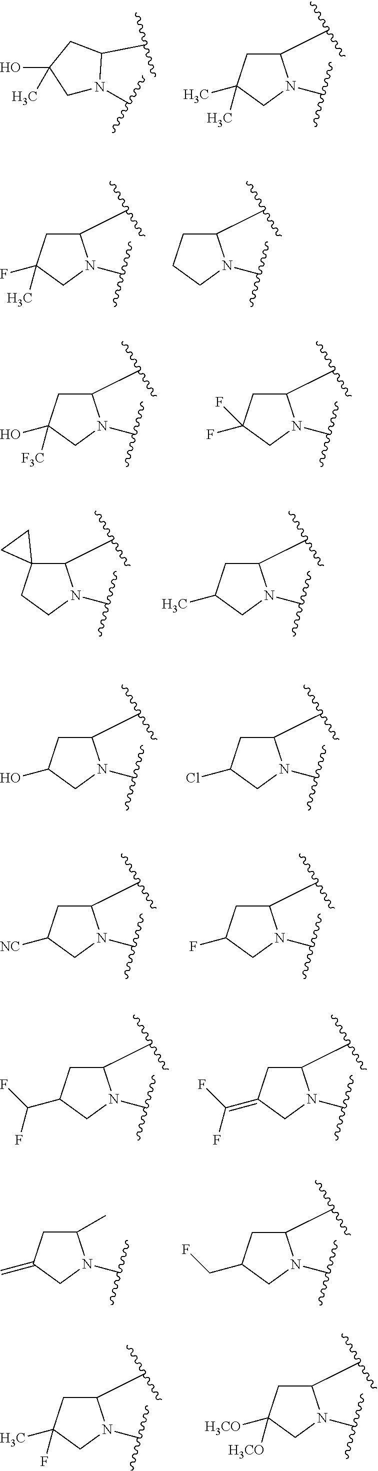

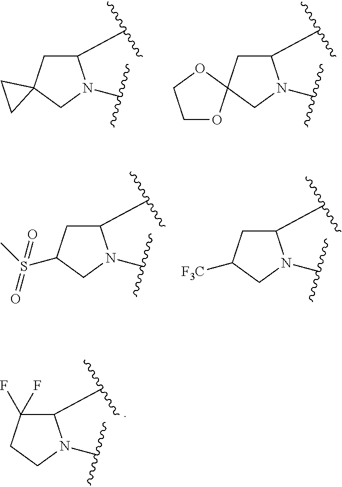 [SMILES: *C1CC2(CC2)CN1* ; *C1CC2(CN1*)OCCO2 ; *C1CC(S(C)(=O)=O)CN1* ; *C1CC(C(F)(F)F)CN1* ; *C1N(*)CCC1(F)F]

In other embodiments of the compounds of Formula II,

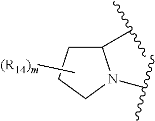

is selected from the groups shown below:

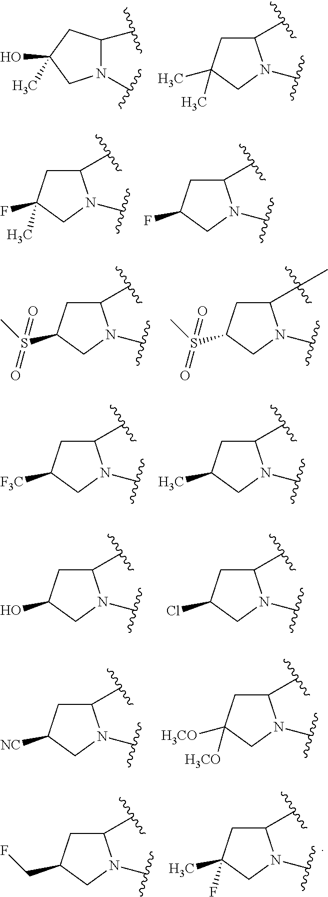

In preferred embodiments of the compounds of Formula II,

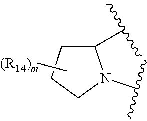

is selected from the groups shown below:

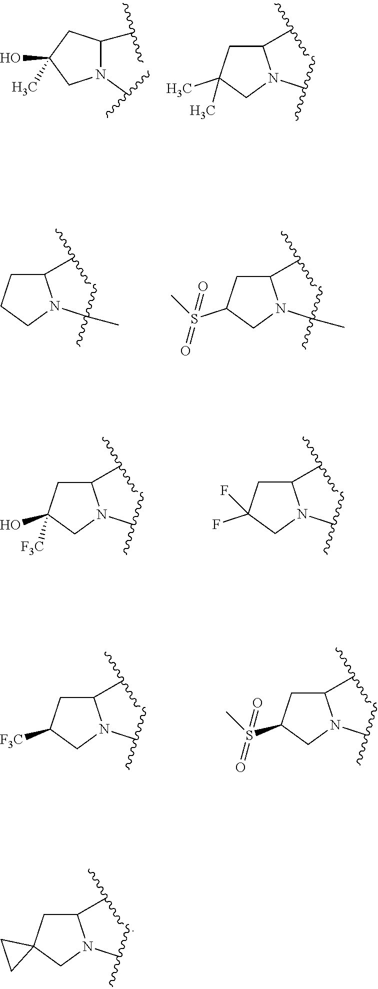

In certain embodiments of the compounds of the invention,

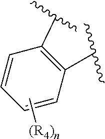

is selected from the groups below:

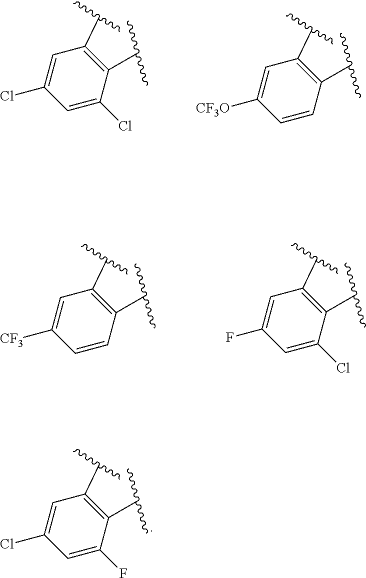

Representative compounds of the invention include the compounds set forth in the table below and pharmaceutically acceptable salts thereof.

| Compound | |
|---|---|
| No. | Structure |
| 1 | 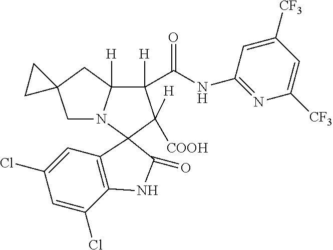 [SMILES: O=C(Nc1cc(C(F)(F)F)cc(C(F)(F)F)n1)C1C2CC3(CC3)CN2C2(C(=O)Nc3c(Cl)cc(Cl)cc32)C1C(=O)O] |
| 2 | 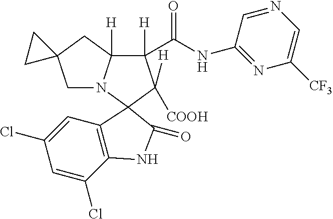 [SMILES: O=C(Nc1cncc(C(F)(F)F)n1)C1C2CC3(CC3)CN2C2(C(=O)Nc3c(Cl)cc(Cl)cc32)C1C(=O)O] |
| 3 | 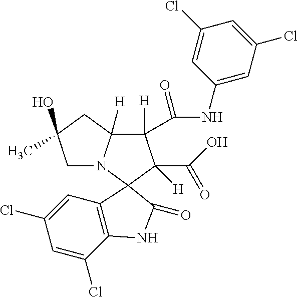 [SMILES: C[C@]1(O)CC2C(C(=O)Nc3cc(Cl)cc(Cl)c3)C(C(=O)O)C3(C(=O)Nc4c(Cl)cc(Cl)cc43)N2C1] |
| 4 | 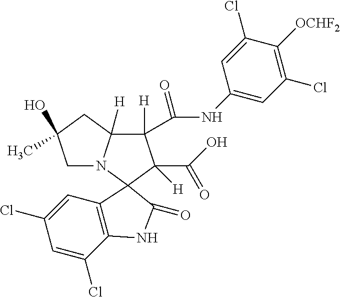 [SMILES: C[C@]1(O)CC2C(C(=O)Nc3cc(Cl)c(OC(F)F)c(Cl)c3)C(C(=O)O)C3(C(=O)Nc4c(Cl)cc(Cl)cc43)N2C1] |
| 5 | 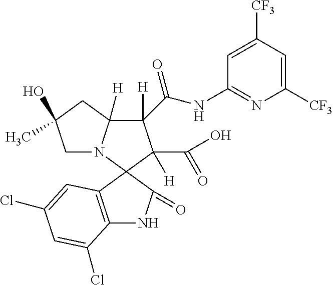 [SMILES: C[C@]1(O)CC2C(C(=O)Nc3cc(C(F)(F)F)cc(C(F)(F)F)n3)C(C(=O)O)C3(C(=O)Nc4c(Cl)cc(Cl)cc43)N2C1] |
| 6 | 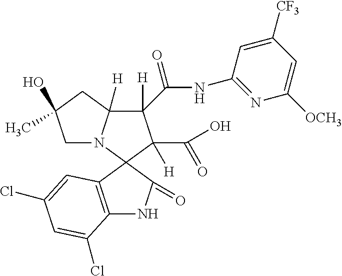 [SMILES: COc1cc(C(F)(F)F)cc(NC(=O)C2C3C[C@](C)(O)CN3C3(C(=O)Nc4c(Cl)cc(Cl)cc43)C2C(=O)O)n1] |
| 7 | 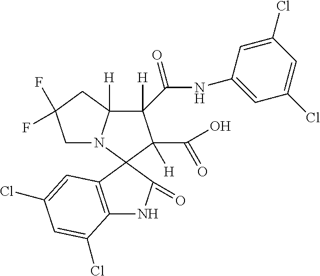 [SMILES: O=C(Nc1cc(Cl)cc(Cl)c1)C1C2CC(F)(F)CN2C2(C(=O)Nc3c(Cl)cc(Cl)cc32)C1C(=O)O] |
| 8 | 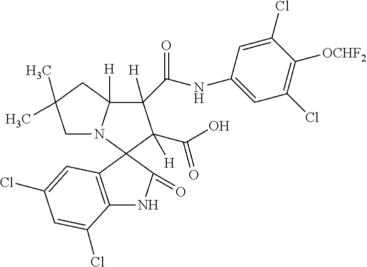 [SMILES: CC1(C)CC2C(C(=O)Nc3cc(Cl)c(OC(F)F)c(Cl)c3)C(C(=O)O)C3(C(=O)Nc4c(Cl)cc(Cl)cc43)N2C1] |
| 9 | 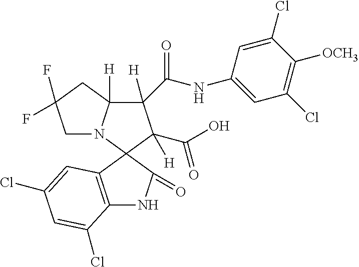 [SMILES: COc1c(Cl)cc(NC(=O)C2C3CC(F)(F)CN3C3(C(=O)Nc4c(Cl)cc(Cl)cc43)C2C(=O)O)cc1Cl] |
| 10 | 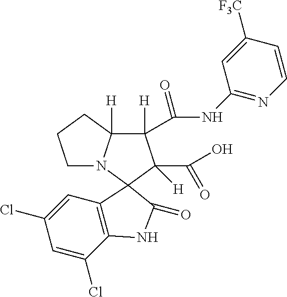 [SMILES: O=C(Nc1cc(C(F)(F)F)ccn1)C1C2CCCN2C2(C(=O)Nc3c(Cl)cc(Cl)cc32)C1C(=O)O] |
| 11 | 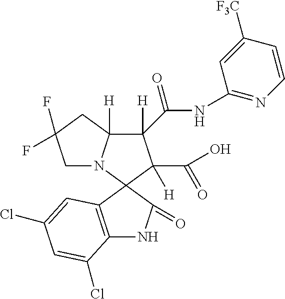 [SMILES: O=C(Nc1cc(C(F)(F)F)ccn1)C1C2CC(F)(F)CN2C2(C(=O)Nc3c(Cl)cc(Cl)cc32)C1C(=O)O] |
| 12 | 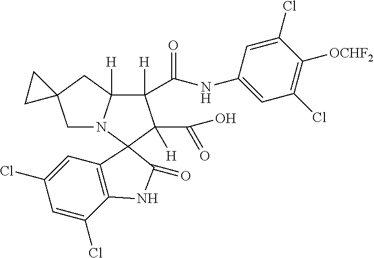 [SMILES: O=C(Nc1cc(Cl)c(OC(F)F)c(Cl)c1)C1C2CC3(CC3)CN2C2(C(=O)Nc3c(Cl)cc(Cl)cc32)C1C(=O)O] |
| 13 | 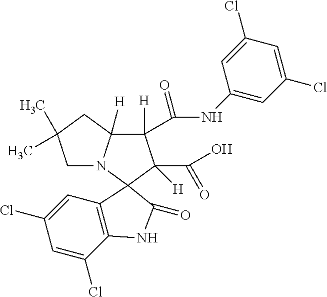 [SMILES: CC1(C)CC2C(C(=O)Nc3cc(Cl)cc(Cl)c3)C(C(=O)O)C3(C(=O)Nc4c(Cl)cc(Cl)cc43)N2C1] |
| 14 | 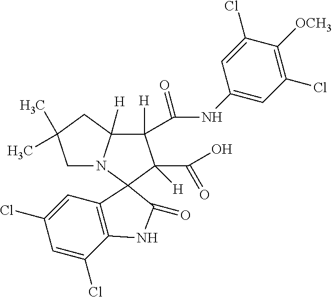 [SMILES: COc1c(Cl)cc(NC(=O)C2C3CC(C)(C)CN3C3(C(=O)Nc4c(Cl)cc(Cl)cc43)C2C(=O)O)cc1Cl] |
| 15 | 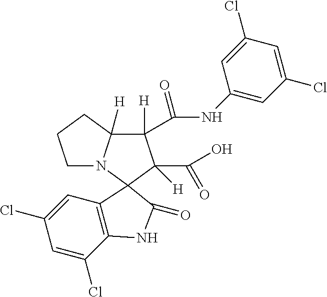 [SMILES: O=C(Nc1cc(Cl)cc(Cl)c1)C1C2CCCN2C2(C(=O)Nc3c(Cl)cc(Cl)cc32)C1C(=O)O] |
| 16 | 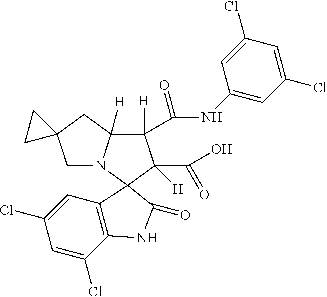 [SMILES: O=C(Nc1cc(Cl)cc(Cl)c1)C1C2CC3(CC3)CN2C2(C(=O)Nc3c(Cl)cc(Cl)cc32)C1C(=O)O] |
| 17 | 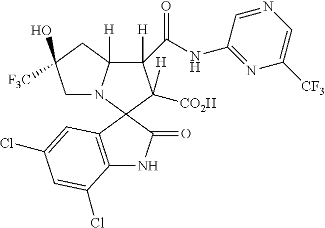 [SMILES: O=C(Nc1cncc(C(F)(F)F)n1)C1C2C[C@@](O)(C(F)(F)F)CN2C2(C(=O)Nc3c(Cl)cc(Cl)cc32)C1C(=O)O] |
| 18 | 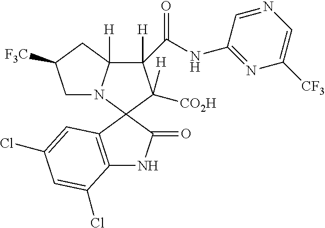 [SMILES: O=C(Nc1cncc(C(F)(F)F)n1)C1C2C[C@H](C(F)(F)F)CN2C2(C(=O)Nc3c(Cl)cc(Cl)cc32)C1C(=O)O] |
| 19 | 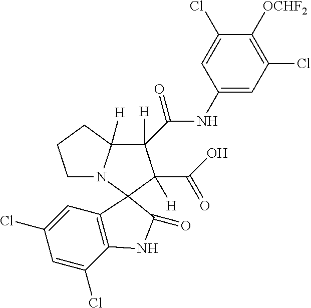 [SMILES: O=C(Nc1cc(Cl)c(OC(F)F)c(Cl)c1)C1C2CCCN2C2(C(=O)Nc3c(Cl)cc(Cl)cc32)C1C(=O)O] |
| 20 | 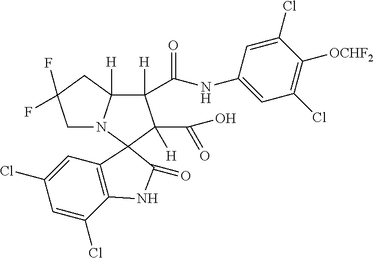 [SMILES: O=C(Nc1cc(Cl)c(OC(F)F)c(Cl)c1)C1C2CC(F)(F)CN2C2(C(=O)Nc3c(Cl)cc(Cl)cc32)C1C(=O)O] |
| 21 | 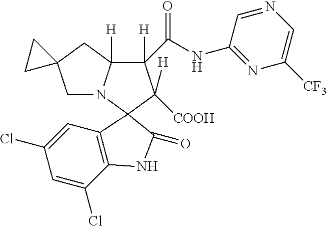 [SMILES: O=C(Nc1cncc(C(F)(F)F)n1)C1C2CC3(CC3)CN2C2(C(=O)Nc3c(Cl)cc(Cl)cc32)C1C(=O)O] |
| 22 | 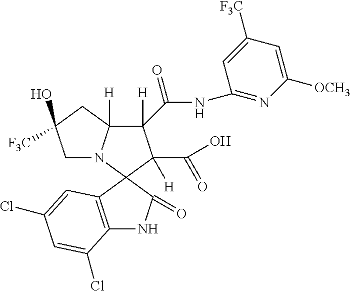 [SMILES: COc1cc(C(F)(F)F)cc(NC(=O)C2C3C[C@@](O)(C(F)(F)F)CN3C3(C(=O)Nc4c(Cl)cc(Cl)cc43)C2C(=O)O)n1] |
| 23 | 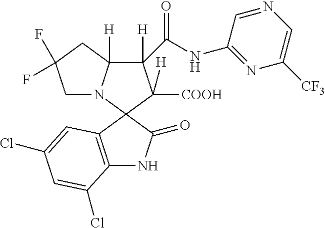 [SMILES: O=C(Nc1cncc(C(F)(F)F)n1)C1C2CC(F)(F)CN2C2(C(=O)Nc3c(Cl)cc(Cl)cc32)C1C(=O)O] |
| 24 | 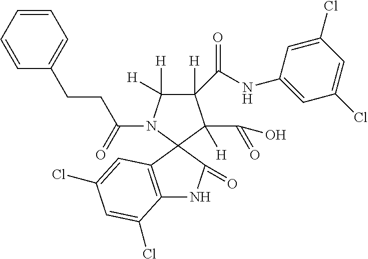 [SMILES: O=C(Nc1cc(Cl)cc(Cl)c1)C1CN(C(=O)CCc2ccccc2)C2(C(=O)Nc3c(Cl)cc(Cl)cc32)C1C(=O)O] |
| 25 | 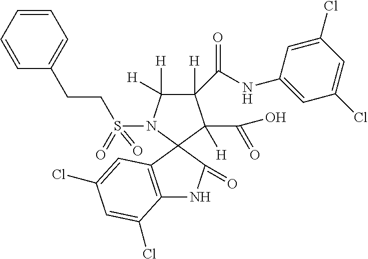 [SMILES: O=C(Nc1cc(Cl)cc(Cl)c1)C1CN(S(=O)(=O)CCc2ccccc2)C2(C(=O)Nc3c(Cl)cc(Cl)cc32)C1C(=O)O] |
| 26 | 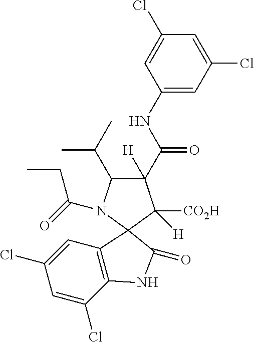 [SMILES: CCC(=O)N1C(C(C)C)C(C(=O)Nc2cc(Cl)cc(Cl)c2)C(C(=O)O)C12C(=O)Nc1c(Cl)cc(Cl)cc12] |
| 27 | 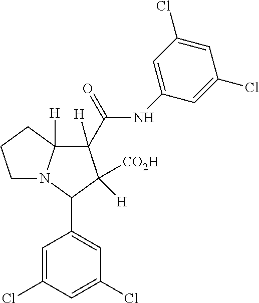 [SMILES: O=C(Nc1cc(Cl)cc(Cl)c1)C1C(C(=O)O)C(c2cc(Cl)cc(Cl)c2)N2CCCC12] |
| 28 | 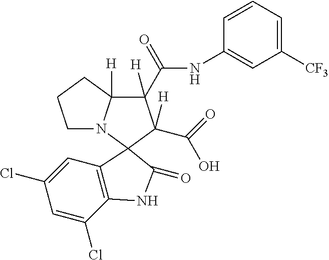 [SMILES: O=C(Nc1cccc(C(F)(F)F)c1)C1C2CCCN2C2(C(=O)Nc3c(Cl)cc(Cl)cc32)C1C(=O)O] |
| 29 | 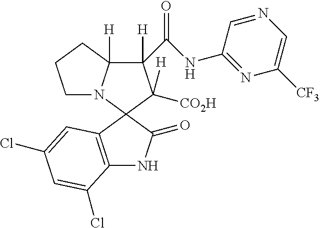 [SMILES: O=C(Nc1cncc(C(F)(F)F)n1)C1C2CCCN2C2(C(=O)Nc3c(Cl)cc(Cl)cc32)C1C(=O)O] |
| 30 | 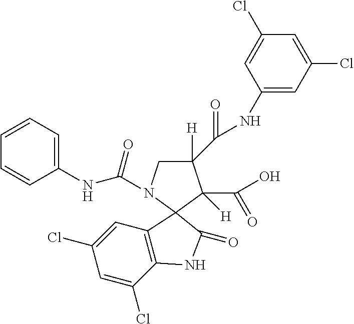 [SMILES: O=C(Nc1cc(Cl)cc(Cl)c1)C1CN(C(=O)Nc2ccccc2)C2(C(=O)Nc3c(Cl)cc(Cl)cc32)C1C(=O)O] |
| 31 | 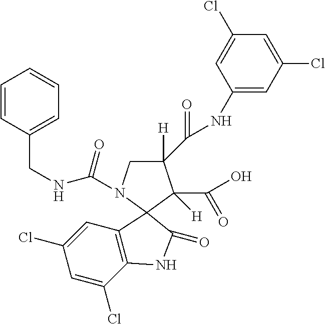 [SMILES: O=C(Nc1cc(Cl)cc(Cl)c1)C1CN(C(=O)NCc2ccccc2)C2(C(=O)Nc3c(Cl)cc(Cl)cc32)C1C(=O)O] |
| 32 | 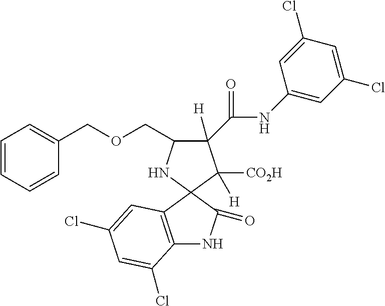 [SMILES: O=C(Nc1cc(Cl)cc(Cl)c1)C1C(COCc2ccccc2)NC2(C(=O)Nc3c(Cl)cc(Cl)cc32)C1C(=O)O] |
| 33 | 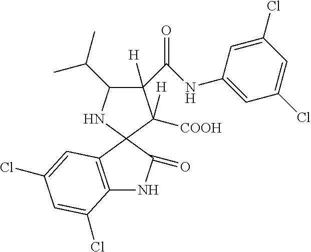 [SMILES: CC(C)C1NC2(C(=O)Nc3c(Cl)cc(Cl)cc32)C(C(=O)O)C1C(=O)Nc1cc(Cl)cc(Cl)c1] |
| 34 | 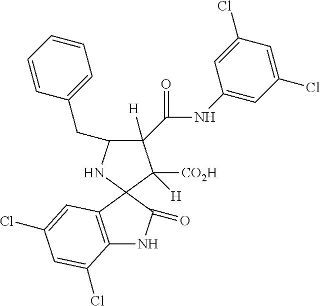 [SMILES: O=C(Nc1cc(Cl)cc(Cl)c1)C1C(Cc2ccccc2)NC2(C(=O)Nc3c(Cl)cc(Cl)cc32)C1C(=O)O] |
| 35 | 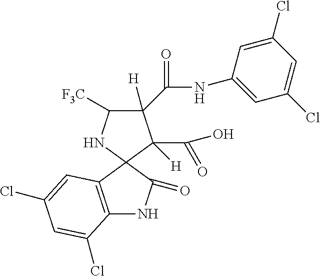 [SMILES: O=C(Nc1cc(Cl)cc(Cl)c1)C1C(C(F)(F)F)NC2(C(=O)Nc3c(Cl)cc(Cl)cc32)C1C(=O)O] |
| 36 | 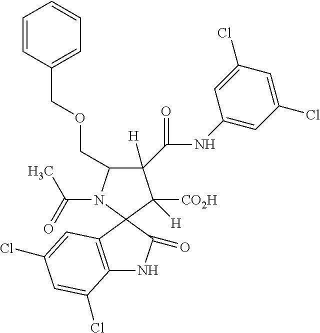 [SMILES: CC(=O)N1C(COCc2ccccc2)C(C(=O)Nc2cc(Cl)cc(Cl)c2)C(C(=O)O)C12C(=O)Nc1c(Cl)cc(Cl)cc12] |
| 37 | 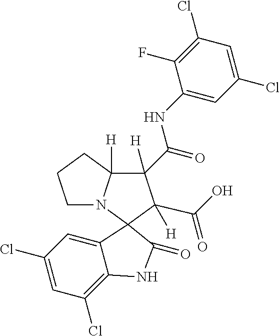 [SMILES: O=C(Nc1cc(Cl)cc(Cl)c1F)C1C2CCCN2C2(C(=O)Nc3c(Cl)cc(Cl)cc32)C1C(=O)O] |
| 38 | 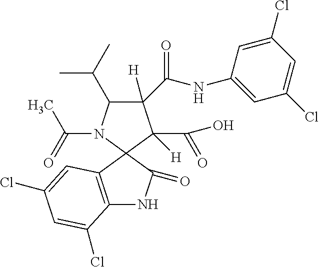 [SMILES: CC(=O)N1C(C(C)C)C(C(=O)Nc2cc(Cl)cc(Cl)c2)C(C(=O)O)C12C(=O)Nc1c(Cl)cc(Cl)cc12] |
| 39 | 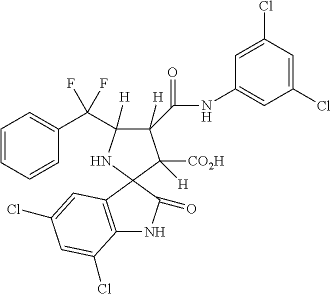 [SMILES: O=C(Nc1cc(Cl)cc(Cl)c1)C1C(C(F)(F)c2ccccc2)NC2(C(=O)Nc3c(Cl)cc(Cl)cc32)C1C(=O)O] |
| 40 | 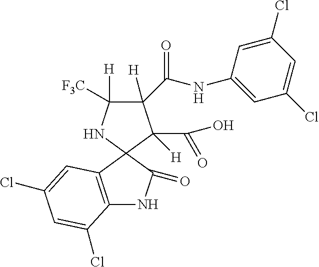 [SMILES: O=C(Nc1cc(Cl)cc(Cl)c1)C1C(C(F)(F)F)NC2(C(=O)Nc3c(Cl)cc(Cl)cc32)C1C(=O)O] |
| 41 | 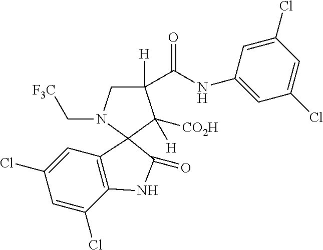 [SMILES: O=C(Nc1cc(Cl)cc(Cl)c1)C1CN(CC(F)(F)F)C2(C(=O)Nc3c(Cl)cc(Cl)cc32)C1C(=O)O] |
| 42 | 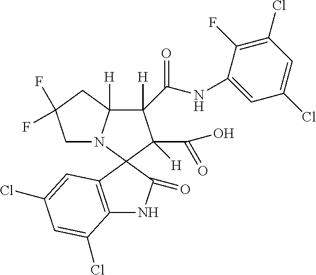 [SMILES: O=C(Nc1cc(Cl)cc(Cl)c1F)C1C2CC(F)(F)CN2C2(C(=O)Nc3c(Cl)cc(Cl)cc32)C1C(=O)O] |
| 43 | 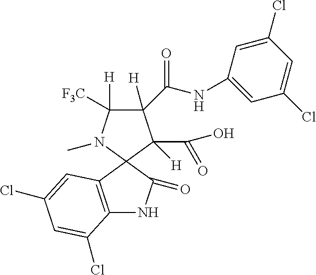 [SMILES: CN1C(C(F)(F)F)C(C(=O)Nc2cc(Cl)cc(Cl)c2)C(C(=O)O)C12C(=O)Nc1c(Cl)cc(Cl)cc12] |
| 44 | 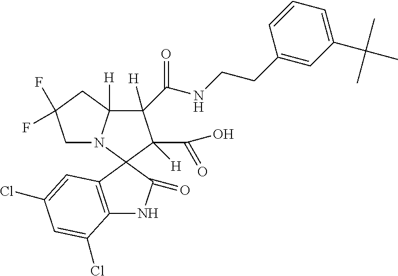 [SMILES: CC(C)(C)c1cccc(CCNC(=O)C2C3CC(F)(F)CN3C3(C(=O)Nc4c(Cl)cc(Cl)cc43)C2C(=O)O)c1] |
| 45 | 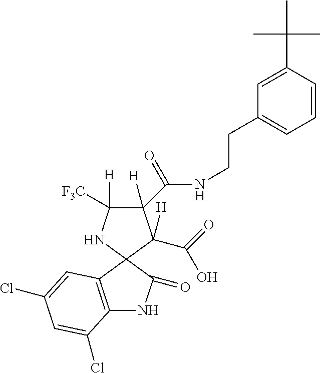 [SMILES: CC(C)(C)c1cccc(CCNC(=O)C2C(C(F)(F)F)NC3(C(=O)Nc4c(Cl)cc(Cl)cc43)C2C(=O)O)c1] |
| 46 | 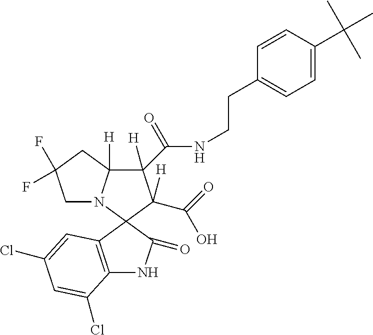 [SMILES: CC(C)(C)c1ccc(CCNC(=O)C2C3CC(F)(F)CN3C3(C(=O)Nc4c(Cl)cc(Cl)cc43)C2C(=O)O)cc1] |
| 47 | 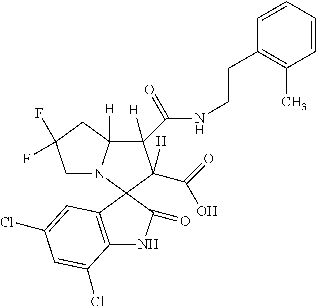 [SMILES: Cc1ccccc1CCNC(=O)C1C2CC(F)(F)CN2C2(C(=O)Nc3c(Cl)cc(Cl)cc32)C1C(=O)O] |
| 48 | 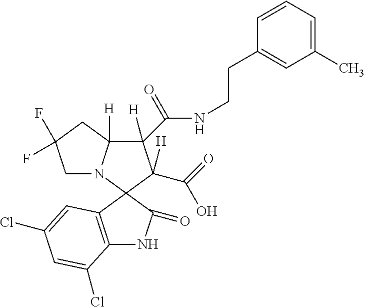 [SMILES: Cc1cccc(CCNC(=O)C2C3CC(F)(F)CN3C3(C(=O)Nc4c(Cl)cc(Cl)cc43)C2C(=O)O)c1] |
| 49 | 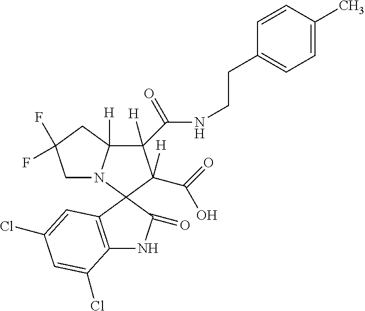 [SMILES: Cc1ccc(CCNC(=O)C2C3CC(F)(F)CN3C3(C(=O)Nc4c(Cl)cc(Cl)cc43)C2C(=O)O)cc1] |
| 50 | 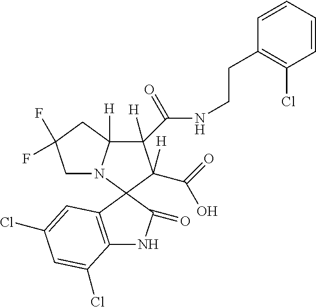 [SMILES: O=C(NCCc1ccccc1Cl)C1C2CC(F)(F)CN2C2(C(=O)Nc3c(Cl)cc(Cl)cc32)C1C(=O)O] |
| 51 | 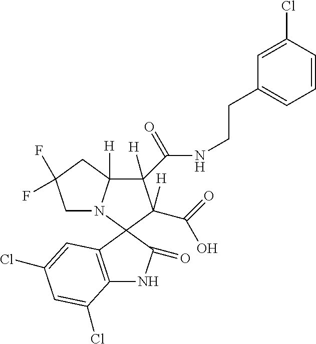 [SMILES: O=C(NCCc1cccc(Cl)c1)C1C2CC(F)(F)CN2C2(C(=O)Nc3c(Cl)cc(Cl)cc32)C1C(=O)O] |
| 52 | 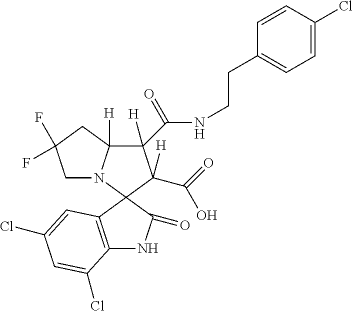 [SMILES: O=C(NCCc1ccc(Cl)cc1)C1C2CC(F)(F)CN2C2(C(=O)Nc3c(Cl)cc(Cl)cc32)C1C(=O)O] |
| 53 | 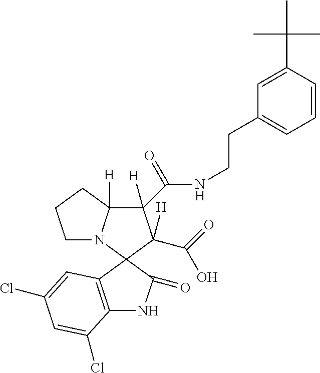 [SMILES: CC(C)(C)c1cccc(CCNC(=O)C2C3CCCN3C3(C(=O)Nc4c(Cl)cc(Cl)cc43)C2C(=O)O)c1] |
| 54 | 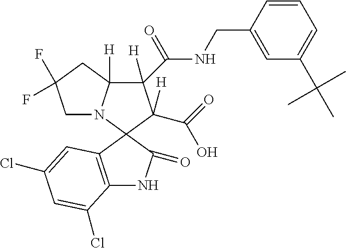 [SMILES: CC(C)(C)c1cccc(CNC(=O)C2C3CC(F)(F)CN3C3(C(=O)Nc4c(Cl)cc(Cl)cc43)C2C(=O)O)c1] |
| 55 | 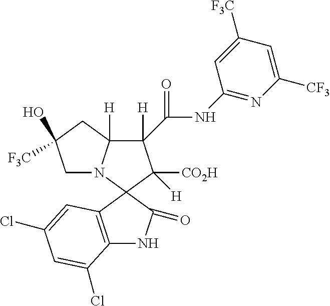 [SMILES: O=C(Nc1cc(C(F)(F)F)cc(C(F)(F)F)n1)C1C2C[C@@](O)(C(F)(F)F)CN2C2(C(=O)Nc3c(Cl)cc(Cl)cc32)C1C(=O)O] |
| 56 | 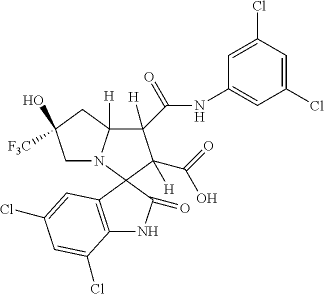 [SMILES: O=C(Nc1cc(Cl)cc(Cl)c1)C1C2C[C@@](O)(C(F)(F)F)CN2C2(C(=O)Nc3c(Cl)cc(Cl)cc32)C1C(=O)O] |
| 57 | 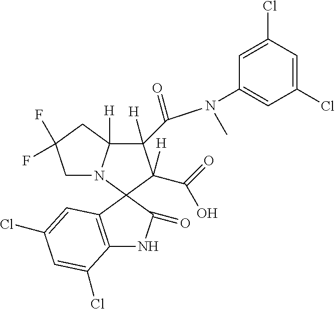 [SMILES: CN(C(=O)C1C2CC(F)(F)CN2C2(C(=O)Nc3c(Cl)cc(Cl)cc32)C1C(=O)O)c1cc(Cl)cc(Cl)c1] |
| 58 | 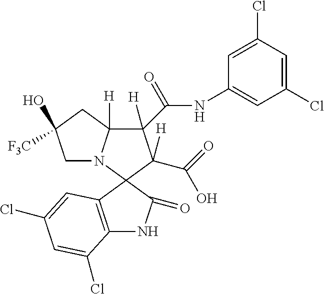 [SMILES: O=C(Nc1cc(Cl)cc(Cl)c1)C1C2C[C@@](O)(C(F)(F)F)CN2C2(C(=O)Nc3c(Cl)cc(Cl)cc32)C1C(=O)O] |
| 59 | 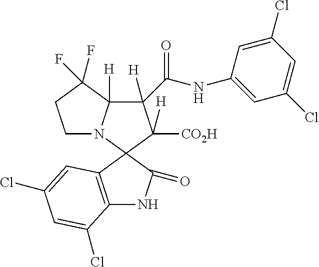 [SMILES: O=C(Nc1cc(Cl)cc(Cl)c1)C1C2N(CCC2(F)F)C2(C(=O)Nc3c(Cl)cc(Cl)cc32)C1C(=O)O] |
| 60 | 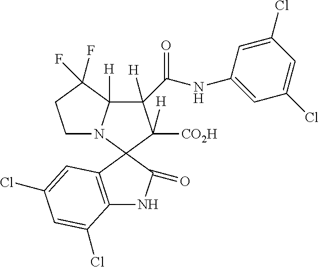 [SMILES: O=C(Nc1cc(Cl)cc(Cl)c1)C1C2N(CCC2(F)F)C2(C(=O)Nc3c(Cl)cc(Cl)cc32)C1C(=O)O] |
| 61 | 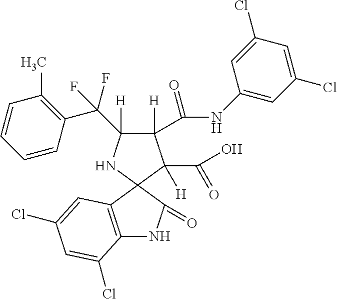 [SMILES: Cc1ccccc1C(F)(F)C1NC2(C(=O)Nc3c(Cl)cc(Cl)cc32)C(C(=O)O)C1C(=O)Nc1cc(Cl)cc(Cl)c1] |
| 62 | 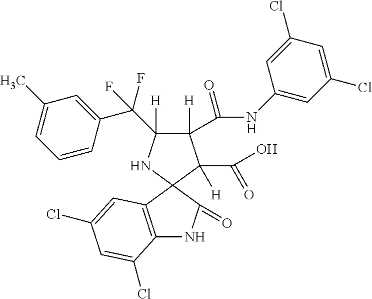 [SMILES: Cc1cccc(C(F)(F)C2NC3(C(=O)Nc4c(Cl)cc(Cl)cc43)C(C(=O)O)C2C(=O)Nc2cc(Cl)cc(Cl)c2)c1] |
| 63 | 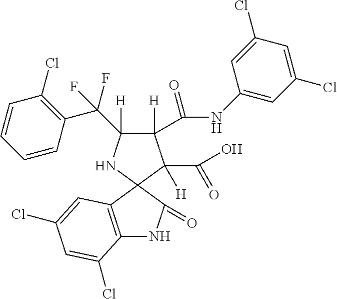 [SMILES: O=C(Nc1cc(Cl)cc(Cl)c1)C1C(C(F)(F)c2ccccc2Cl)NC2(C(=O)Nc3c(Cl)cc(Cl)cc32)C1C(=O)O] |
| 64 | 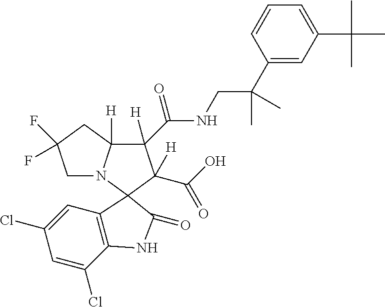 [SMILES: CC(C)(C)c1cccc(C(C)(C)CNC(=O)C2C3CC(F)(F)CN3C3(C(=O)Nc4c(Cl)cc(Cl)cc43)C2C(=O)O)c1] |
| 65 | 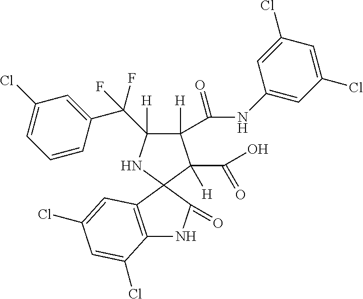 [SMILES: O=C(Nc1cc(Cl)cc(Cl)c1)C1C(C(F)(F)c2cccc(Cl)c2)NC2(C(=O)Nc3c(Cl)cc(Cl)cc32)C1C(=O)O] |
| 66.9a 66.9b.1 66.9b.2 66.9b.3 | 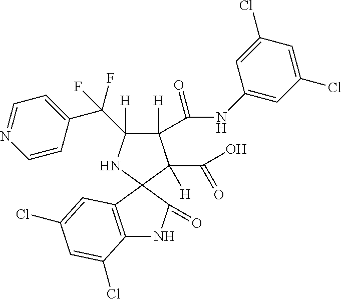 [SMILES: O=C(Nc1cc(Cl)cc(Cl)c1)C1C(C(F)(F)c2ccncc2)NC2(C(=O)Nc3c(Cl)cc(Cl)cc32)C1C(=O)O] |
| 67.9a 67.9b | 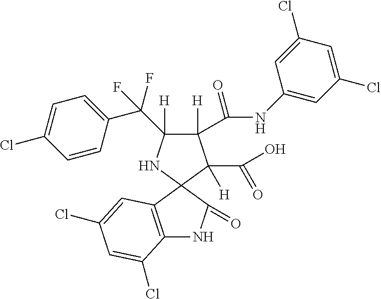 [SMILES: O=C(Nc1cc(Cl)cc(Cl)c1)C1C(C(F)(F)c2ccc(Cl)cc2)NC2(C(=O)Nc3c(Cl)cc(Cl)cc32)C1C(=O)O] |
| 68.a 68.b 68.c | 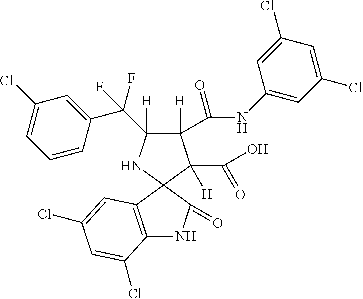 [SMILES: O=C(Nc1cc(Cl)cc(Cl)c1)C1C(C(F)(F)c2cccc(Cl)c2)NC2(C(=O)Nc3c(Cl)cc(Cl)cc32)C1C(=O)O] |
| 69.a 69.b | 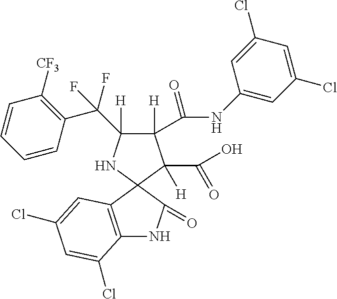 [SMILES: O=C(Nc1cc(Cl)cc(Cl)c1)C1C(C(F)(F)c2ccccc2C(F)(F)F)NC2(C(=O)Nc3c(Cl)cc(Cl)cc32)C1C(=O)O] |
| 70.a 70.b 70.c | 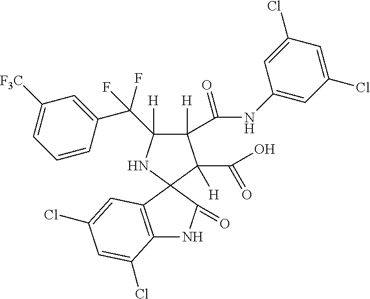 [SMILES: O=C(Nc1cc(Cl)cc(Cl)c1)C1C(C(F)(F)c2cccc(C(F)(F)F)c2)NC2(C(=O)Nc3c(Cl)cc(Cl)cc32)C1C(=O)O] |
| 71.a | 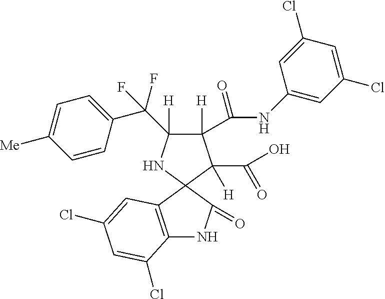 [SMILES: Cc1ccc(C(F)(F)C2NC3(C(=O)Nc4c(Cl)cc(Cl)cc43)C(C(=O)O)C2C(=O)Nc2cc(Cl)cc(Cl)c2)cc1] |
| 72.a 72.b 72.c | 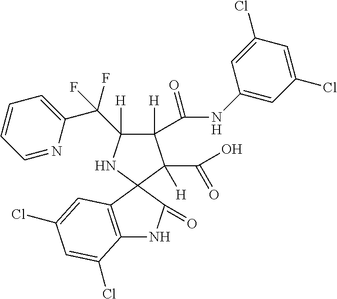 [SMILES: O=C(Nc1cc(Cl)cc(Cl)c1)C1C(C(F)(F)c2ccccn2)NC2(C(=O)Nc3c(Cl)cc(Cl)cc32)C1C(=O)O] |
| 73.a 73.b 73.c | 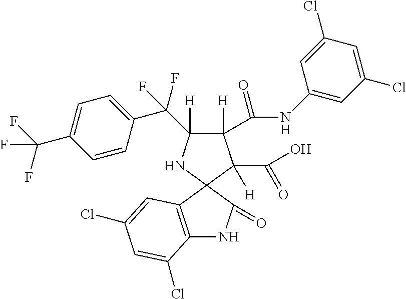 [SMILES: O=C(Nc1cc(Cl)cc(Cl)c1)C1C(C(F)(F)c2ccc(C(F)(F)F)cc2)NC2(C(=O)Nc3c(Cl)cc(Cl)cc32)C1C(=O)O] |
| 74.a | 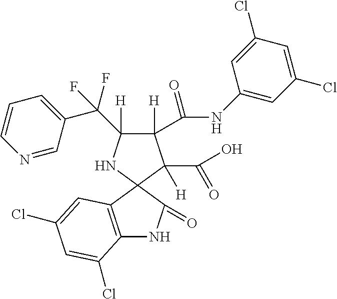 [SMILES: O=C(Nc1cc(Cl)cc(Cl)c1)C1C(C(F)(F)c2cccnc2)NC2(C(=O)Nc3c(Cl)cc(Cl)cc32)C1C(=O)O] |
| 75.a | 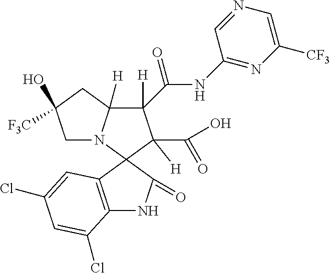 [SMILES: O=C(Nc1cncc(C(F)(F)F)n1)C1C2C[C@@](O)(C(F)(F)F)CN2C2(C(=O)Nc3c(Cl)cc(Cl)cc32)C1C(=O)O] |
| 76.a 76.b 76.c 76.d | 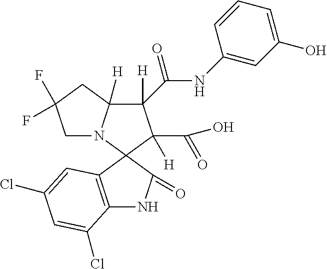 [SMILES: O=C(Nc1cccc(O)c1)C1C2CC(F)(F)CN2C2(C(=O)Nc3c(Cl)cc(Cl)cc32)C1C(=O)O] |
| 77.a 77.b | 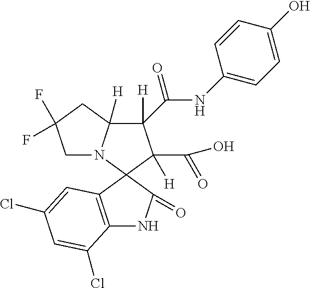 [SMILES: O=C(Nc1ccc(O)cc1)C1C2CC(F)(F)CN2C2(C(=O)Nc3c(Cl)cc(Cl)cc32)C1C(=O)O] |
| 78.a | 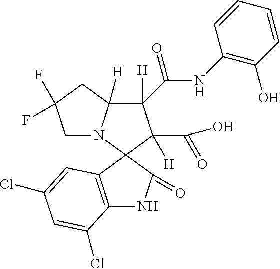 [SMILES: O=C(Nc1ccccc1O)C1C2CC(F)(F)CN2C2(C(=O)Nc3c(Cl)cc(Cl)cc32)C1C(=O)O] |
| 79a 79b 79c | 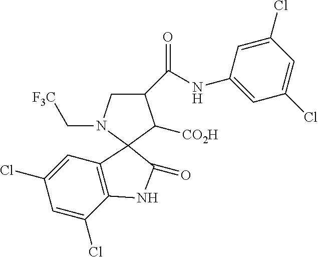 [SMILES: O=C(Nc1cc(Cl)cc(Cl)c1)C1CN(CC(F)(F)F)C2(C(=O)Nc3c(Cl)cc(Cl)cc32)C1C(=O)O] |
| 80.7a.1 80.7a.2 80.7b.1 80.7b.2 | 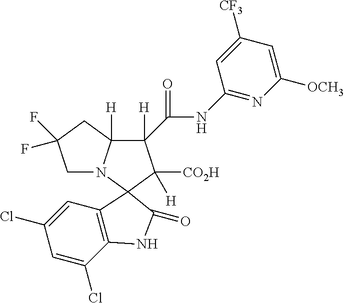 [SMILES: COc1cc(C(F)(F)F)cc(NC(=O)C2C3CC(F)(F)CN3C3(C(=O)Nc4c(Cl)cc(Cl)cc43)C2C(=O)O)n1] |
| 81 | 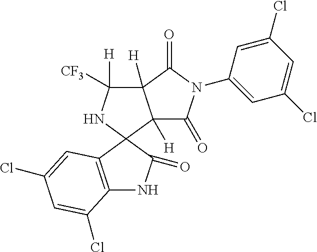 [SMILES: O=C1C2C(C(F)(F)F)NC3(C(=O)Nc4c(Cl)cc(Cl)cc43)C2C(=O)N1c1cc(Cl)cc(Cl)c1] |
| 82.6a | 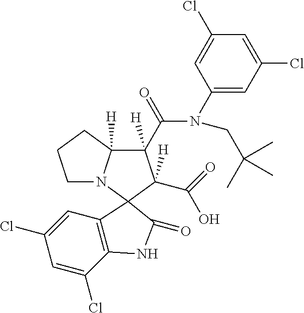 [SMILES: CC(C)(C)CN(C(=O)[C@H]1[C@@H](C(=O)O)C2(C(=O)Nc3c(Cl)cc(Cl)cc32)N2CCC[C@@H]12)c1cc(Cl)cc(Cl)c1] |
| 82.6b | 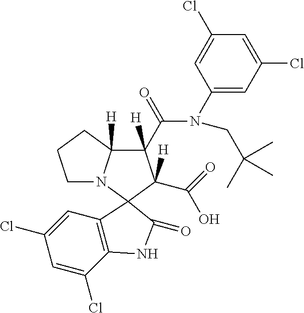 [SMILES: CC(C)(C)CN(C(=O)[C@H]1[C@H]2CCCN2C2(C(=O)Nc3c(Cl)cc(Cl)cc32)[C@H]1C(=O)O)c1cc(Cl)cc(Cl)c1] |
| 83a | 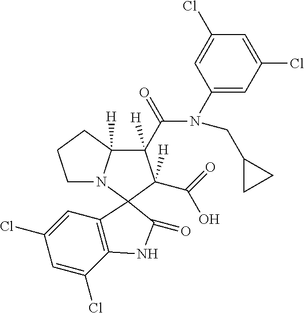 [SMILES: O=C(O)[C@@H]1[C@H](C(=O)N(CC2CC2)c2cc(Cl)cc(Cl)c2)[C@@H]2CCCN2C12C(=O)Nc1c(Cl)cc(Cl)cc12] |
| 83b | 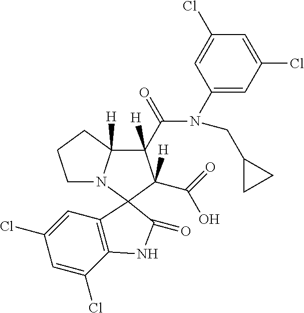 [SMILES: O=C(O)[C@H]1[C@@H](C(=O)N(CC2CC2)c2cc(Cl)cc(Cl)c2)[C@H]2CCCN2C12C(=O)Nc1c(Cl)cc(Cl)cc12] |
| 84a | 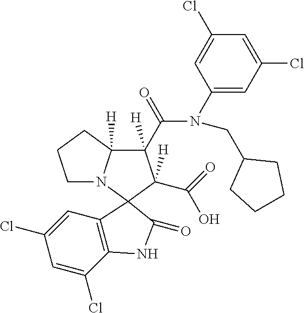 [SMILES: O=C(O)[C@@H]1[C@H](C(=O)N(CC2CCCC2)c2cc(Cl)cc(Cl)c2)[C@@H]2CCCN2C12C(=O)Nc1c(Cl)cc(Cl)cc12] |
| 84b | 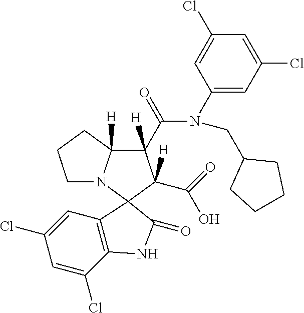 [SMILES: O=C(O)[C@H]1[C@@H](C(=O)N(CC2CCCC2)c2cc(Cl)cc(Cl)c2)[C@H]2CCCN2C12C(=O)Nc1c(Cl)cc(Cl)cc12] |
| 85a | 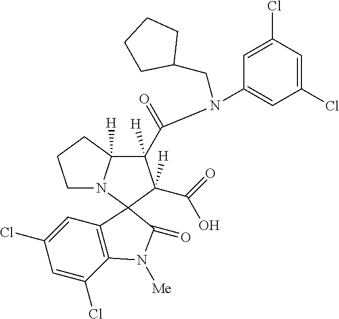 [SMILES: CN1C(=O)C2(c3cc(Cl)cc(Cl)c31)[C@H](C(=O)O)[C@H](C(=O)N(CC1CCCC1)c1cc(Cl)cc(Cl)c1)[C@@H]1CCCN12] |
| 85b | 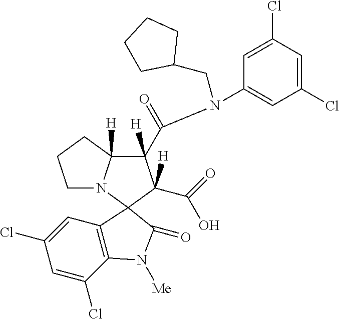 [SMILES: CN1C(=O)C2(c3cc(Cl)cc(Cl)c31)[C@@H](C(=O)O)[C@@H](C(=O)N(CC1CCCC1)c1cc(Cl)cc(Cl)c1)[C@H]1CCCN12] |
| 90 | 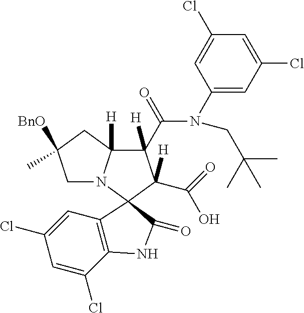 [SMILES: CC(C)(C)CN(C(=O)[C@H]1[C@H]2C[C@](C)(OCc3ccccc3)CN2[C@]2(C(=O)Nc3c(Cl)cc(Cl)cc32)[C@H]1C(=O)O)c1cc(Cl)cc(Cl)c1] |
| 91 | 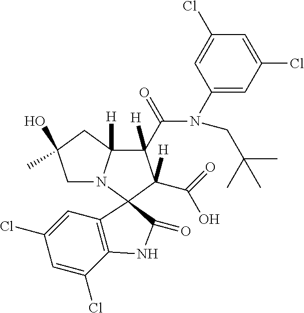 [SMILES: CC(C)(C)CN(C(=O)[C@H]1[C@H]2C[C@](C)(O)CN2[C@]2(C(=O)Nc3c(Cl)cc(Cl)cc32)[C@H]1C(=O)O)c1cc(Cl)cc(Cl)c1] |
| 92 | 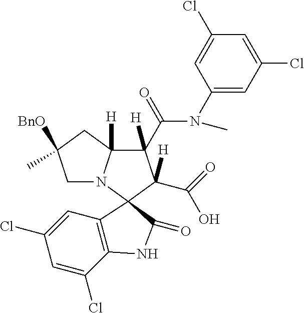 [SMILES: CN(C(=O)[C@H]1[C@H]2C[C@](C)(OCc3ccccc3)CN2[C@]2(C(=O)Nc3c(Cl)cc(Cl)cc32)[C@H]1C(=O)O)c1cc(Cl)cc(Cl)c1] |
| 93 | 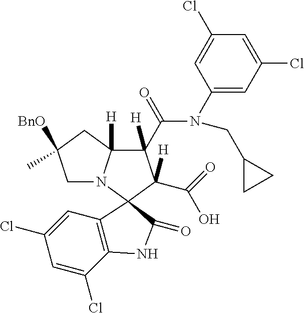 [SMILES: C[C@]1(OCc2ccccc2)C[C@@H]2[C@H](C(=O)N(CC3CC3)c3cc(Cl)cc(Cl)c3)[C@H](C(=O)O)[C@@]3(C(=O)Nc4c(Cl)cc(Cl)cc43)N2C1] |
| 94 | 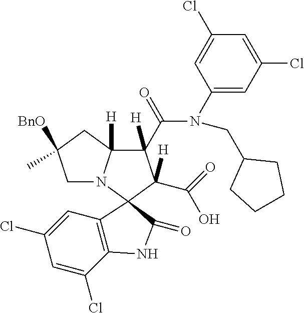 [SMILES: C[C@]1(OCc2ccccc2)C[C@@H]2[C@H](C(=O)N(CC3CCCC3)c3cc(Cl)cc(Cl)c3)[C@H](C(=O)O)[C@@]3(C(=O)Nc4c(Cl)cc(Cl)cc43)N2C1] |
| 95 | 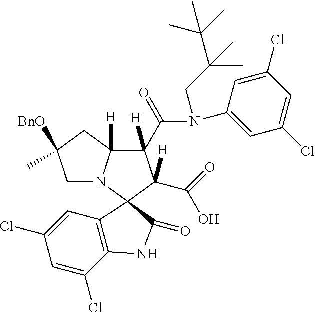 [SMILES: CC(C)(C)C(C)(C)CN(C(=O)[C@H]1[C@H]2C[C@](C)(OCc3ccccc3)CN2[C@]2(C(=O)Nc3c(Cl)cc(Cl)cc32)[C@H]1C(=O)O)c1cc(Cl)cc(Cl)c1] |
| 96 | 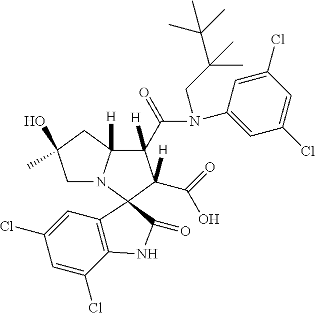 [SMILES: CC(C)(C)C(C)(C)CN(C(=O)[C@H]1[C@H]2C[C@](C)(O)CN2[C@]2(C(=O)Nc3c(Cl)cc(Cl)cc32)[C@H]1C(=O)O)c1cc(Cl)cc(Cl)c1] |
| 97 | 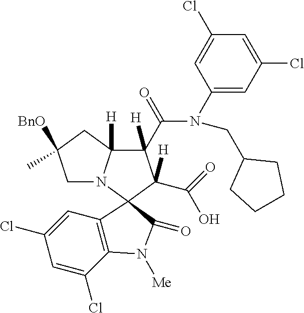 [SMILES: CN1C(=O)[C@]2(c3cc(Cl)cc(Cl)c31)[C@@H](C(=O)O)[C@@H](C(=O)N(CC1CCCC1)c1cc(Cl)cc(Cl)c1)[C@H]1C[C@](C)(OCc3ccccc3)CN12] |
| 100.6b | 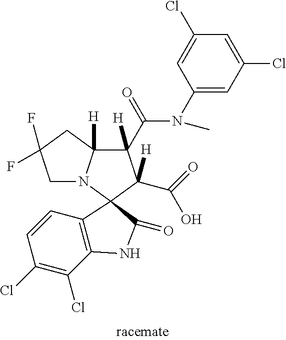 [SMILES: CN(C(=O)[C@H]1[C@H]2CC(F)(F)CN2[C@]2(C(=O)Nc3c2ccc(Cl)c3Cl)[C@H]1C(=O)O)c1cc(Cl)cc(Cl)c1] |
| 101 | 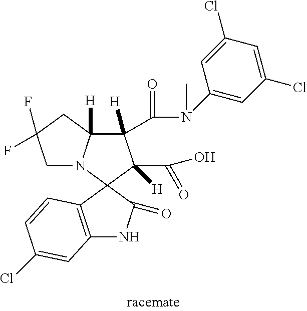 [SMILES: CN(C(=O)[C@H]1[C@H]2CC(F)(F)CN2C2(C(=O)Nc3cc(Cl)ccc32)[C@H]1C(=O)O)c1cc(Cl)cc(Cl)c1] |
| 102 | 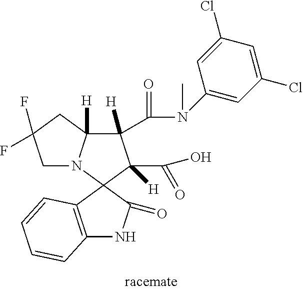 [SMILES: CN(C(=O)[C@H]1[C@H]2CC(F)(F)CN2C2(C(=O)Nc3ccccc32)[C@H]1C(=O)O)c1cc(Cl)cc(Cl)c1] |
| 103 | 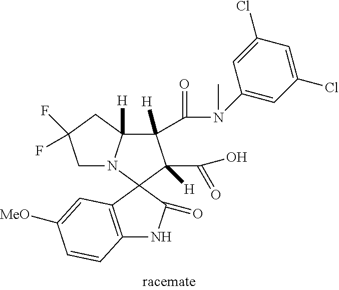 [SMILES: COc1ccc2c(c1)C1(C(=O)N2)[C@@H](C(=O)O)[C@@H](C(=O)N(C)c2cc(Cl)cc(Cl)c2)[C@H]2CC(F)(F)CN21] |
| 104 | 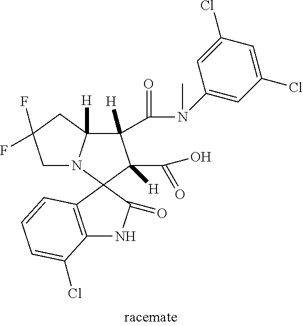 [SMILES: CN(C(=O)[C@H]1[C@H]2CC(F)(F)CN2C2(C(=O)Nc3c(Cl)cccc32)[C@H]1C(=O)O)c1cc(Cl)cc(Cl)c1] |
| 110 | 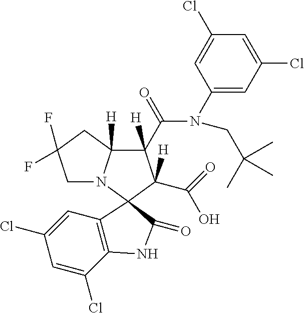 [SMILES: CC(C)(C)CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 111 | 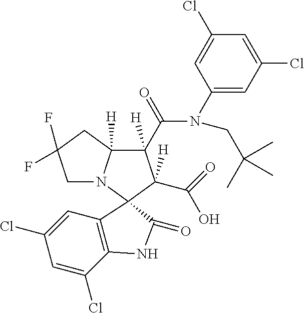 [SMILES: CC(C)(C)CN(C(=O)[C@H]1[C@@H](C(=O)O)[C@]2(C(=O)Nc3c(Cl)cc(Cl)cc32)N2CC(F)(F)C[C@@H]12)c1cc(Cl)cc(Cl)c1] |
| 112 | 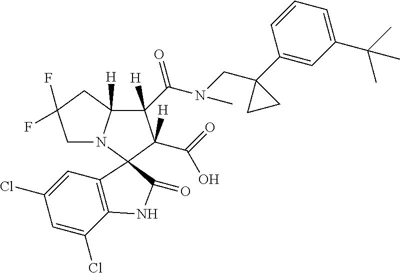 [SMILES: CN(CC1(c2cccc(C(C)(C)C)c2)CC1)C(=O)[C@H]1[C@H]2CC(F)(F)CN2[C@]2(C(=O)Nc3c(Cl)cc(Cl)cc32)[C@H]1C(=O)O] |
| 113 | 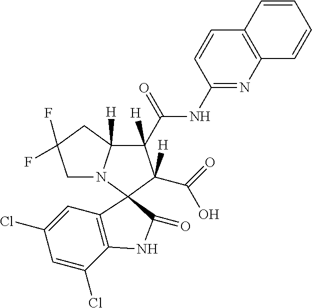 [SMILES: O=C(Nc1ccc2ccccc2n1)[C@H]1[C@H]2CC(F)(F)CN2[C@]2(C(=O)Nc3c(Cl)cc(Cl)cc32)[C@H]1C(=O)O] |
| 114 | 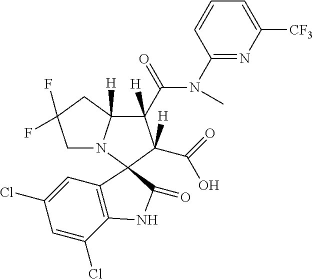 [SMILES: CN(C(=O)[C@H]1[C@H]2CC(F)(F)CN2[C@]2(C(=O)Nc3c(Cl)cc(Cl)cc32)[C@H]1C(=O)O)c1cccc(C(F)(F)F)n1] |
| 115 | 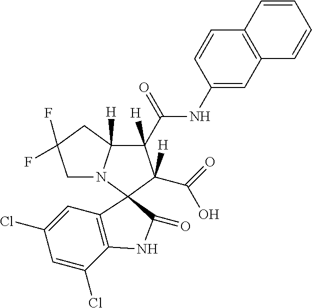 [SMILES: O=C(Nc1ccc2ccccc2c1)[C@H]1[C@H]2CC(F)(F)CN2[C@]2(C(=O)Nc3c(Cl)cc(Cl)cc32)[C@H]1C(=O)O] |
| 116 | 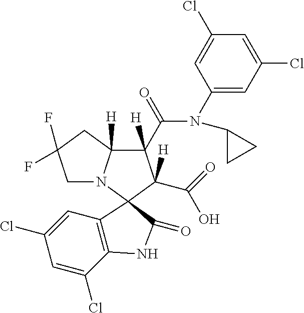 [SMILES: O=C(O)[C@H]1[C@@H](C(=O)N(c2cc(Cl)cc(Cl)c2)C2CC2)[C@H]2CC(F)(F)CN2[C@]12C(=O)Nc1c(Cl)cc(Cl)cc12] |
| 117 | 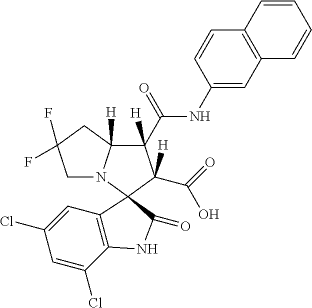 [SMILES: O=C(Nc1ccc2ccccc2c1)[C@H]1[C@H]2CC(F)(F)CN2[C@]2(C(=O)Nc3c(Cl)cc(Cl)cc32)[C@H]1C(=O)O] |
| 118 | 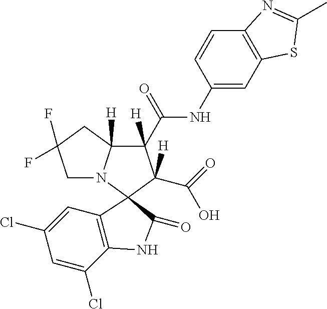 [SMILES: Cc1nc2ccc(NC(=O)[C@H]3[C@H]4CC(F)(F)CN4[C@]4(C(=O)Nc5c(Cl)cc(Cl)cc54)[C@H]3C(=O)O)cc2s1] |
| 119 | 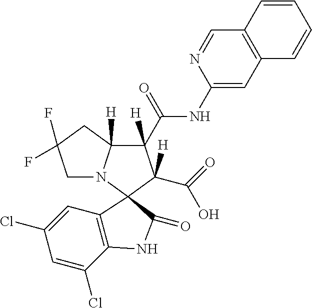 [SMILES: O=C(Nc1cc2ccccc2cn1)[C@H]1[C@H]2CC(F)(F)CN2[C@]2(C(=O)Nc3c(Cl)cc(Cl)cc32)[C@H]1C(=O)O] |
| 120 | 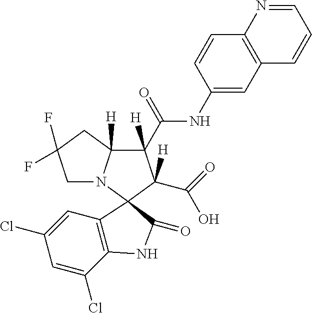 [SMILES: O=C(Nc1ccc2ncccc2c1)[C@H]1[C@H]2CC(F)(F)CN2[C@]2(C(=O)Nc3c(Cl)cc(Cl)cc32)[C@H]1C(=O)O] |
| 121 | 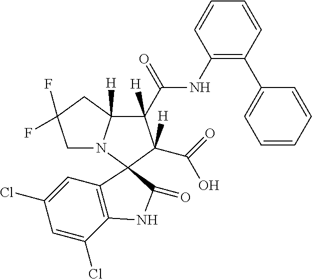 [SMILES: O=C(Nc1ccccc1-c1ccccc1)[C@H]1[C@H]2CC(F)(F)CN2[C@]2(C(=O)Nc3c(Cl)cc(Cl)cc32)[C@H]1C(=O)O] |
| 122 | 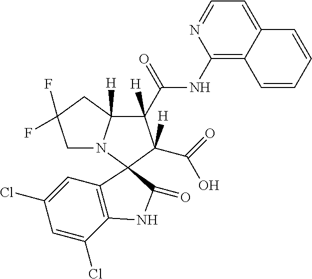 [SMILES: O=C(Nc1nccc2ccccc12)[C@H]1[C@H]2CC(F)(F)CN2[C@]2(C(=O)Nc3c(Cl)cc(Cl)cc32)[C@H]1C(=O)O] |
| 123 | 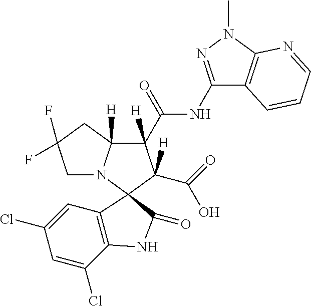 [SMILES: Cn1nc(NC(=O)[C@H]2[C@H]3CC(F)(F)CN3[C@]3(C(=O)Nc4c(Cl)cc(Cl)cc43)[C@H]2C(=O)O)c2cccnc21] |
| 124 | 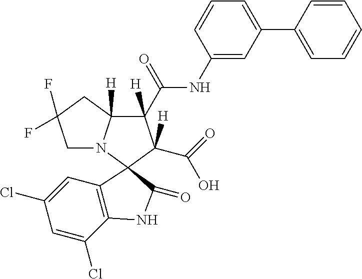 [SMILES: O=C(Nc1cccc(-c2ccccc2)c1)[C@H]1[C@H]2CC(F)(F)CN2[C@]2(C(=O)Nc3c(Cl)cc(Cl)cc32)[C@H]1C(=O)O] |
| 125 | 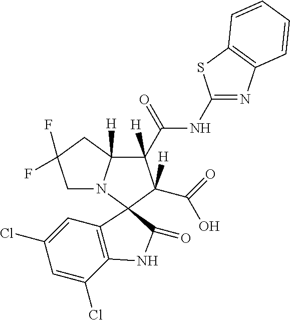 [SMILES: O=C(Nc1nc2ccccc2s1)[C@H]1[C@H]2CC(F)(F)CN2[C@]2(C(=O)Nc3c(Cl)cc(Cl)cc32)[C@H]1C(=O)O] |
| 126 | 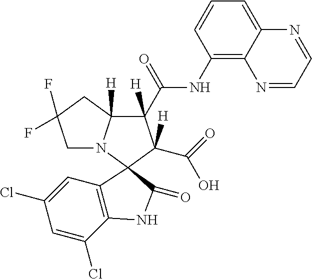 [SMILES: O=C(Nc1cccc2nccnc12)[C@H]1[C@H]2CC(F)(F)CN2[C@]2(C(=O)Nc3c(Cl)cc(Cl)cc32)[C@H]1C(=O)O] |
| 127 | 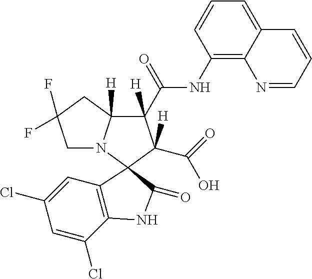 [SMILES: O=C(Nc1cccc2cccnc12)[C@H]1[C@H]2CC(F)(F)CN2[C@]2(C(=O)Nc3c(Cl)cc(Cl)cc32)[C@H]1C(=O)O] |
| 128 | 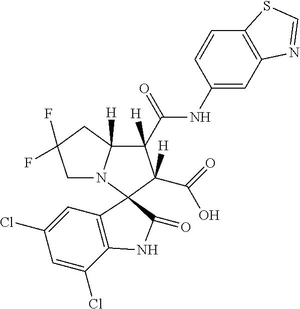 [SMILES: O=C(Nc1ccc2scnc2c1)[C@H]1[C@H]2CC(F)(F)CN2[C@]2(C(=O)Nc3c(Cl)cc(Cl)cc32)[C@H]1C(=O)O] |
| 129 | 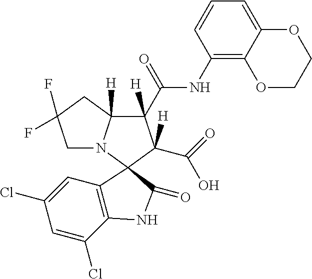 [SMILES: O=C(Nc1cccc2c1OCCO2)[C@H]1[C@H]2CC(F)(F)CN2[C@]2(C(=O)Nc3c(Cl)cc(Cl)cc32)[C@H]1C(=O)O] |
| 130 | 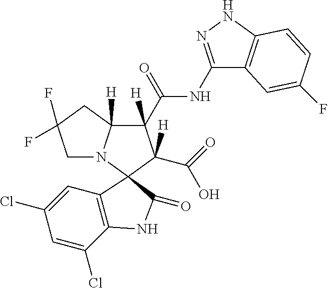 [SMILES: O=C(Nc1n[nH]c2ccc(F)cc12)[C@H]1[C@H]2CC(F)(F)CN2[C@]2(C(=O)Nc3c(Cl)cc(Cl)cc32)[C@H]1C(=O)O] |
| 131 | 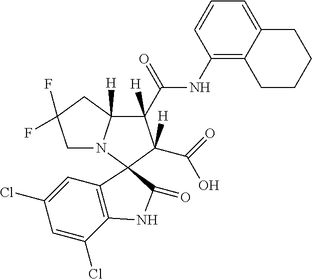 [SMILES: O=C(Nc1cccc2c1CCCC2)[C@H]1[C@H]2CC(F)(F)CN2[C@]2(C(=O)Nc3c(Cl)cc(Cl)cc32)[C@H]1C(=O)O] |
| 132 | 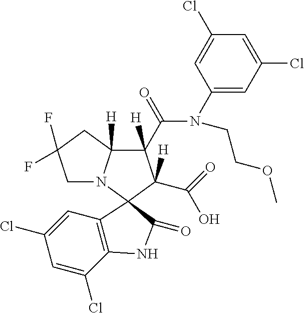 [SMILES: COCCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 133 | 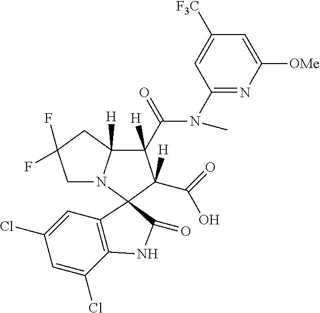 [SMILES: COc1cc(C(F)(F)F)cc(N(C)C(=O)[C@H]2[C@H]3CC(F)(F)CN3[C@]3(C(=O)Nc4c(Cl)cc(Cl)cc43)[C@H]2C(=O)O)n1] |
| 134 | 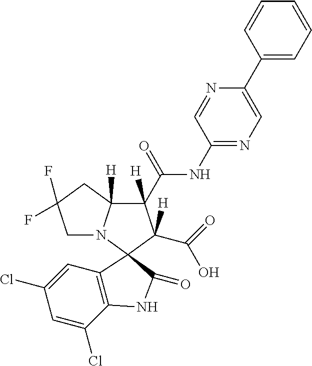 [SMILES: O=C(Nc1cnc(-c2ccccc2)cn1)[C@H]1[C@H]2CC(F)(F)CN2[C@]2(C(=O)Nc3c(Cl)cc(Cl)cc32)[C@H]1C(=O)O] |
| 135 | 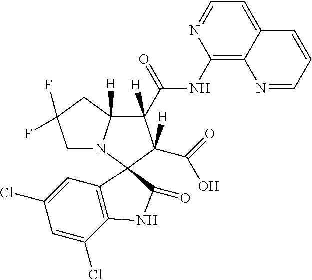 [SMILES: O=C(Nc1nccc2cccnc12)[C@H]1[C@H]2CC(F)(F)CN2[C@]2(C(=O)Nc3c(Cl)cc(Cl)cc32)[C@H]1C(=O)O] |
| 136 | 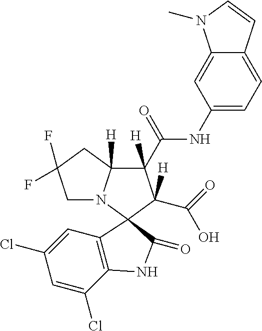 [SMILES: Cn1ccc2ccc(NC(=O)[C@H]3[C@H]4CC(F)(F)CN4[C@]4(C(=O)Nc5c(Cl)cc(Cl)cc54)[C@H]3C(=O)O)cc21] |
| 137 | 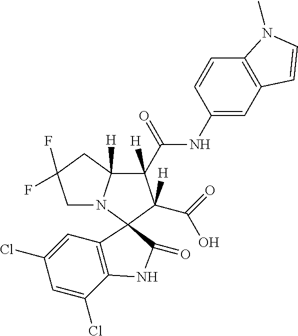 [SMILES: Cn1ccc2cc(NC(=O)[C@H]3[C@H]4CC(F)(F)CN4[C@]4(C(=O)Nc5c(Cl)cc(Cl)cc54)[C@H]3C(=O)O)ccc21] |
| 138 | 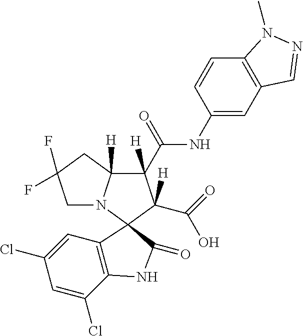 [SMILES: Cn1ncc2cc(NC(=O)[C@H]3[C@H]4CC(F)(F)CN4[C@]4(C(=O)Nc5c(Cl)cc(Cl)cc54)[C@H]3C(=O)O)ccc21] |
| 139 | 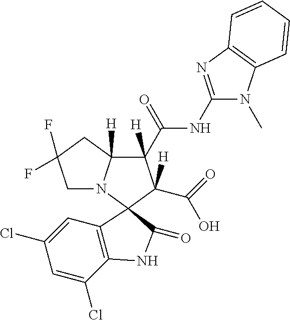 [SMILES: Cn1c(NC(=O)[C@H]2[C@H]3CC(F)(F)CN3[C@]3(C(=O)Nc4c(Cl)cc(Cl)cc43)[C@H]2C(=O)O)nc2ccccc21] |
| 140 | 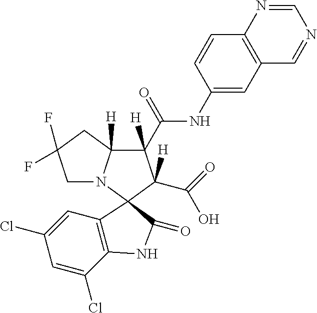 [SMILES: O=C(Nc1ccc2ncncc2c1)[C@H]1[C@H]2CC(F)(F)CN2[C@]2(C(=O)Nc3c(Cl)cc(Cl)cc32)[C@H]1C(=O)O] |
| 141 | 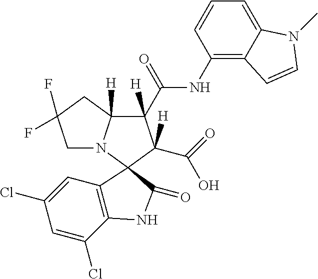 [SMILES: Cn1ccc2c(NC(=O)[C@H]3[C@H]4CC(F)(F)CN4[C@]4(C(=O)Nc5c(Cl)cc(Cl)cc54)[C@H]3C(=O)O)cccc21] |
| 142 | 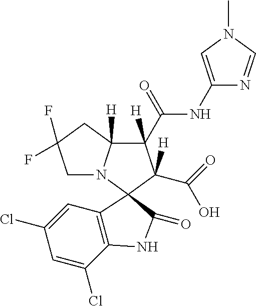 [SMILES: Cn1cnc(NC(=O)[C@H]2[C@H]3CC(F)(F)CN3[C@]3(C(=O)Nc4c(Cl)cc(Cl)cc43)[C@H]2C(=O)O)c1] |
| 143 | 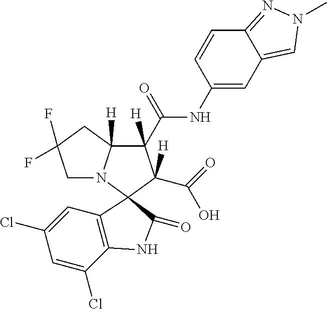 [SMILES: Cn1cc2cc(NC(=O)[C@H]3[C@H]4CC(F)(F)CN4[C@]4(C(=O)Nc5c(Cl)cc(Cl)cc54)[C@H]3C(=O)O)ccc2n1] |
| 144 | 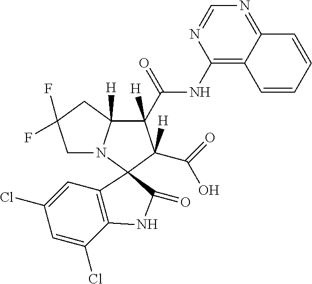 [SMILES: O=C(Nc1ncnc2ccccc12)[C@H]1[C@H]2CC(F)(F)CN2[C@]2(C(=O)Nc3c(Cl)cc(Cl)cc32)[C@H]1C(=O)O] |
| 145 | 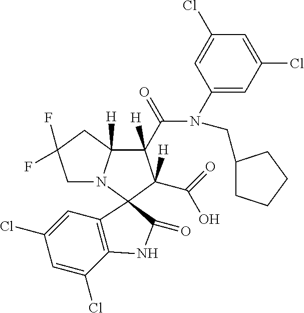 [SMILES: O=C(O)[C@H]1[C@@H](C(=O)N(CC2CCCC2)c2cc(Cl)cc(Cl)c2)[C@H]2CC(F)(F)CN2[C@]12C(=O)Nc1c(Cl)cc(Cl)cc12] |
| 146 | 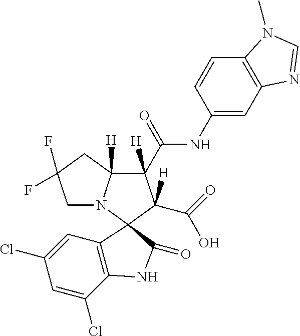 [SMILES: Cn1cnc2cc(NC(=O)[C@H]3[C@H]4CC(F)(F)CN4[C@]4(C(=O)Nc5c(Cl)cc(Cl)cc54)[C@H]3C(=O)O)ccc21] |
| 147 | 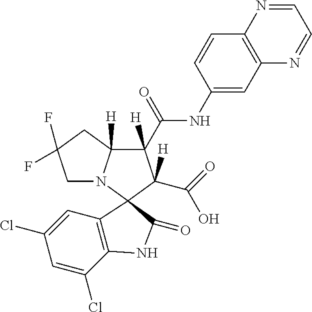 [SMILES: O=C(Nc1ccc2nccnc2c1)[C@H]1[C@H]2CC(F)(F)CN2[C@]2(C(=O)Nc3c(Cl)cc(Cl)cc32)[C@H]1C(=O)O] |
| 148 | 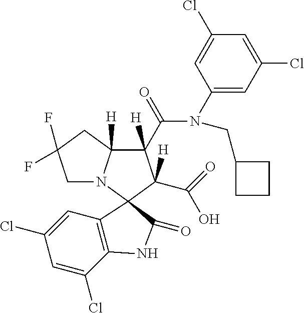 [SMILES: O=C(O)[C@H]1[C@@H](C(=O)N(CC2CCC2)c2cc(Cl)cc(Cl)c2)[C@H]2CC(F)(F)CN2[C@]12C(=O)Nc1c(Cl)cc(Cl)cc12] |
| 149 | 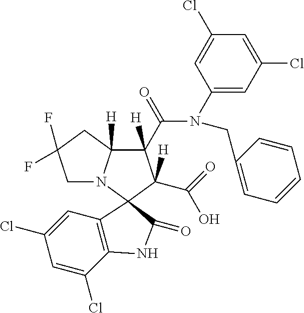 [SMILES: O=C(O)[C@H]1[C@@H](C(=O)N(Cc2ccccc2)c2cc(Cl)cc(Cl)c2)[C@H]2CC(F)(F)CN2[C@]12C(=O)Nc1c(Cl)cc(Cl)cc12] |
| 150 | 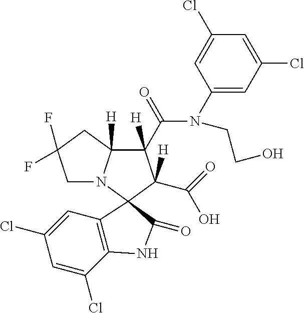 [SMILES: O=C(O)[C@H]1[C@@H](C(=O)N(CCO)c2cc(Cl)cc(Cl)c2)[C@H]2CC(F)(F)CN2[C@]12C(=O)Nc1c(Cl)cc(Cl)cc12] |
| 151 | 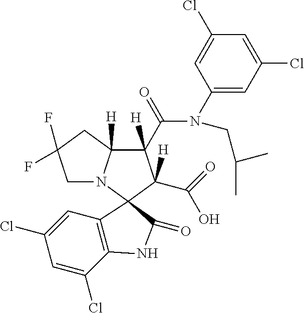 [SMILES: CC(C)CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 152 | 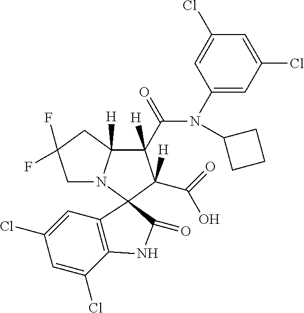 [SMILES: O=C(O)[C@H]1[C@@H](C(=O)N(c2cc(Cl)cc(Cl)c2)C2CCC2)[C@H]2CC(F)(F)CN2[C@]12C(=O)Nc1c(Cl)cc(Cl)cc12] |
| 153 | 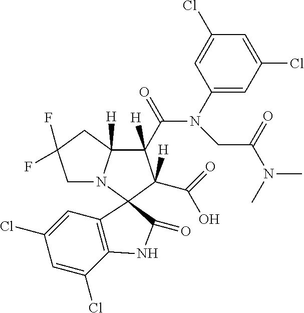 [SMILES: CN(C)C(=O)CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 154 | 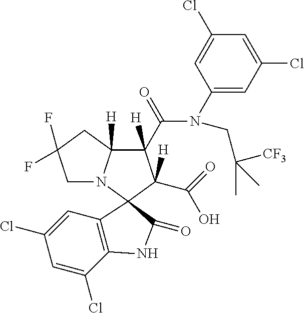 [SMILES: CC(C)(CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1)C(F)(F)F] |
| 155 | 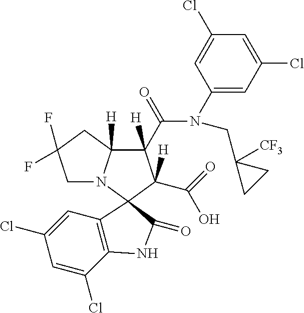 [SMILES: O=C(O)[C@H]1[C@@H](C(=O)N(CC2(C(F)(F)F)CC2)c2cc(Cl)cc(Cl)c2)[C@H]2CC(F)(F)CN2[C@]12C(=O)Nc1c(Cl)cc(Cl)cc12] |
| 156 | 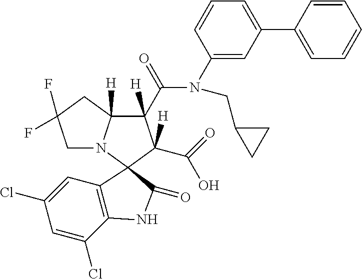 [SMILES: O=C(O)[C@H]1[C@@H](C(=O)N(CC2CC2)c2cccc(-c3ccccc3)c2)[C@H]2CC(F)(F)CN2[C@]12C(=O)Nc1c(Cl)cc(Cl)cc12] |
| 157 | 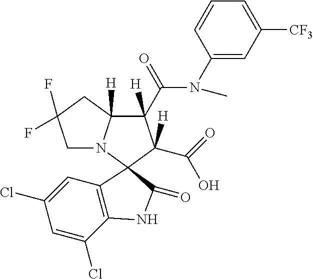 [SMILES: CN(C(=O)[C@H]1[C@H]2CC(F)(F)CN2[C@]2(C(=O)Nc3c(Cl)cc(Cl)cc32)[C@H]1C(=O)O)c1cccc(C(F)(F)F)c1] |
| 158 | 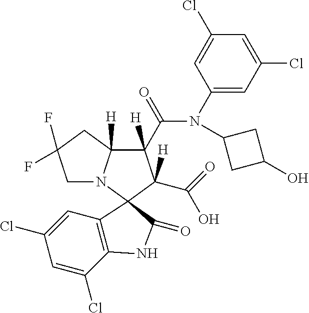 [SMILES: O=C(O)[C@H]1[C@@H](C(=O)N(c2cc(Cl)cc(Cl)c2)C2CC(O)C2)[C@H]2CC(F)(F)CN2[C@]12C(=O)Nc1c(Cl)cc(Cl)cc12] |
| 159 | 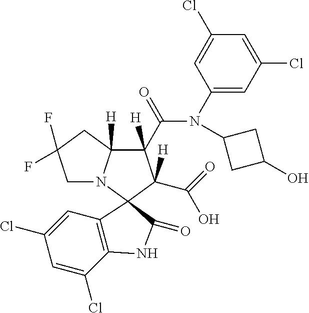 [SMILES: O=C(O)[C@H]1[C@@H](C(=O)N(c2cc(Cl)cc(Cl)c2)C2CC(O)C2)[C@H]2CC(F)(F)CN2[C@]12C(=O)Nc1c(Cl)cc(Cl)cc12] |
| 160 | 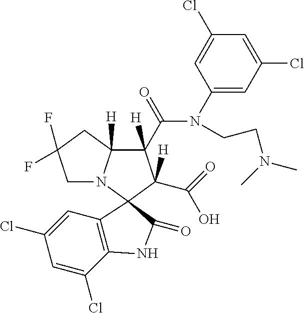 [SMILES: CN(C)CCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 161 | 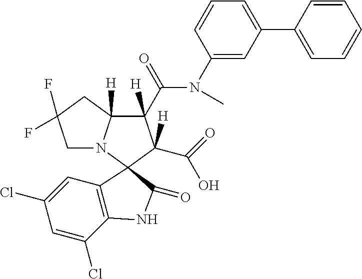 [SMILES: CN(C(=O)[C@H]1[C@H]2CC(F)(F)CN2[C@]2(C(=O)Nc3c(Cl)cc(Cl)cc32)[C@H]1C(=O)O)c1cccc(-c2ccccc2)c1] |
| 162 | 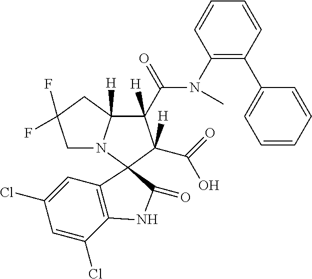 [SMILES: CN(C(=O)[C@H]1[C@H]2CC(F)(F)CN2[C@]2(C(=O)Nc3c(Cl)cc(Cl)cc32)[C@H]1C(=O)O)c1ccccc1-c1ccccc1] |
| 163 | 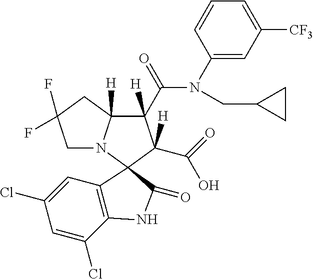 [SMILES: O=C(O)[C@H]1[C@@H](C(=O)N(CC2CC2)c2cccc(C(F)(F)F)c2)[C@H]2CC(F)(F)CN2[C@]12C(=O)Nc1c(Cl)cc(Cl)cc12] |
| 164 | 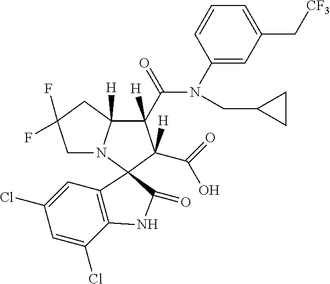 [SMILES: O=C(O)[C@H]1[C@@H](C(=O)N(CC2CC2)c2cccc(CC(F)(F)F)c2)[C@H]2CC(F)(F)CN2[C@]12C(=O)Nc1c(Cl)cc(Cl)cc12] |
| 165 | 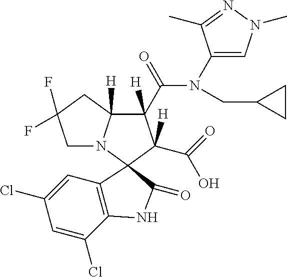 [SMILES: Cc1nn(C)cc1N(CC1CC1)C(=O)[C@H]1[C@H]2CC(F)(F)CN2[C@]2(C(=O)Nc3c(Cl)cc(Cl)cc32)[C@H]1C(=O)O] |
| 166 | 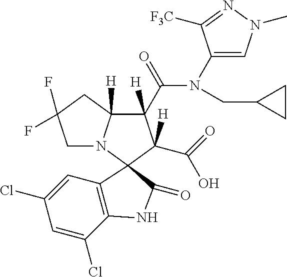 [SMILES: Cn1cc(N(CC2CC2)C(=O)[C@H]2[C@H]3CC(F)(F)CN3[C@]3(C(=O)Nc4c(Cl)cc(Cl)cc43)[C@H]2C(=O)O)c(C(F)(F)F)n1] |
| 167 | 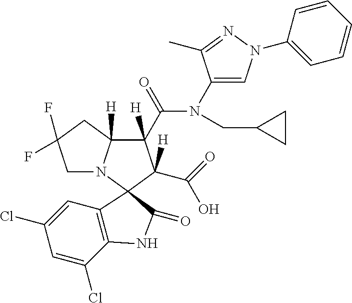 [SMILES: Cc1nn(-c2ccccc2)cc1N(CC1CC1)C(=O)[C@H]1[C@H]2CC(F)(F)CN2[C@]2(C(=O)Nc3c(Cl)cc(Cl)cc32)[C@H]1C(=O)O] |
| 168 | 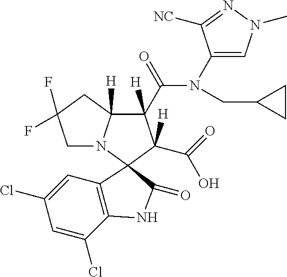 [SMILES: Cn1cc(N(CC2CC2)C(=O)[C@H]2[C@H]3CC(F)(F)CN3[C@]3(C(=O)Nc4c(Cl)cc(Cl)cc43)[C@H]2C(=O)O)c(C#N)n1] |
| 169 | 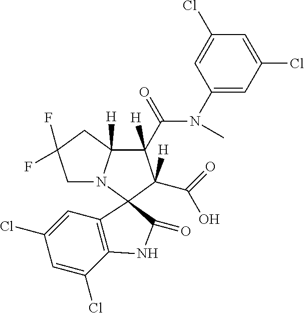 [SMILES: CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 170 | 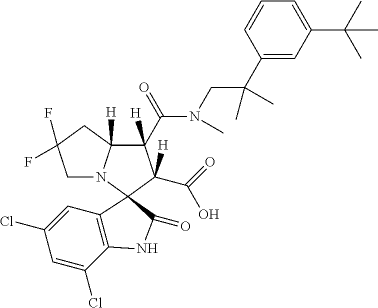 [SMILES: CN(CC(C)(C)c1cccc(C(C)(C)C)c1)C(=O)[C@H]1[C@H]2CC(F)(F)CN2[C@]2(C(=O)Nc3c(Cl)cc(Cl)cc32)[C@H]1C(=O)O] |
| 171 | 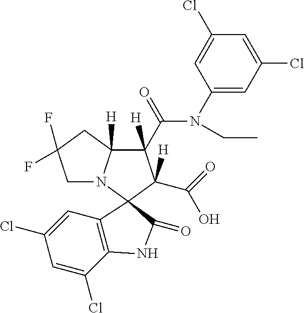 [SMILES: CCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 172 | 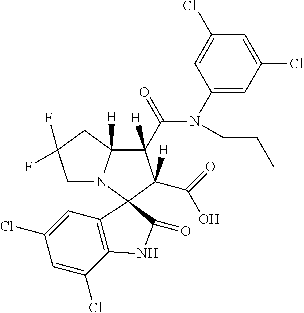 [SMILES: CCCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 173 | 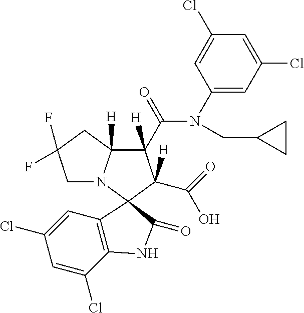 [SMILES: O=C(O)[C@H]1[C@@H](C(=O)N(CC2CC2)c2cc(Cl)cc(Cl)c2)[C@H]2CC(F)(F)CN2[C@]12C(=O)Nc1c(Cl)cc(Cl)cc12] |
| 174 | 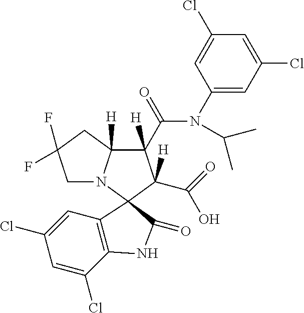 [SMILES: CC(C)N(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 175 | 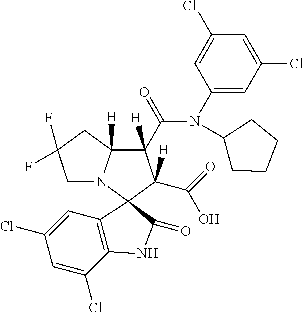 [SMILES: O=C(O)[C@H]1[C@@H](C(=O)N(c2cc(Cl)cc(Cl)c2)C2CCCC2)[C@H]2CC(F)(F)CN2[C@]12C(=O)Nc1c(Cl)cc(Cl)cc12] |
| 176 | 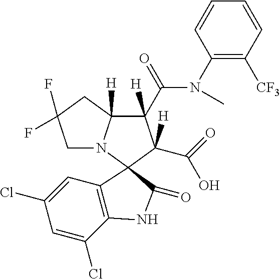 [SMILES: CN(C(=O)[C@H]1[C@H]2CC(F)(F)CN2[C@]2(C(=O)Nc3c(Cl)cc(Cl)cc32)[C@H]1C(=O)O)c1ccccc1C(F)(F)F] |
| 177 | 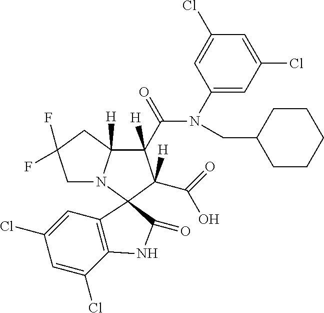 [SMILES: O=C(O)[C@H]1[C@@H](C(=O)N(CC2CCCCC2)c2cc(Cl)cc(Cl)c2)[C@H]2CC(F)(F)CN2[C@]12C(=O)Nc1c(Cl)cc(Cl)cc12] |
| 178 | 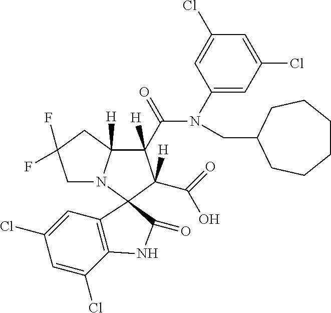 [SMILES: O=C(O)[C@H]1[C@@H](C(=O)N(CC2CCCCCC2)c2cc(Cl)cc(Cl)c2)[C@H]2CC(F)(F)CN2[C@]12C(=O)Nc1c(Cl)cc(Cl)cc12] |
| 179 | 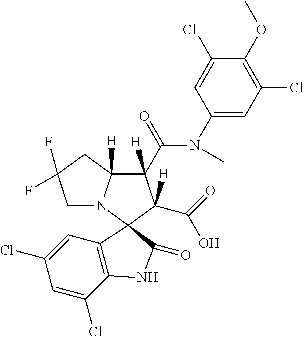 [SMILES: COc1c(Cl)cc(N(C)C(=O)[C@H]2[C@H]3CC(F)(F)CN3[C@]3(C(=O)Nc4c(Cl)cc(Cl)cc43)[C@H]2C(=O)O)cc1Cl] |
| 180 | 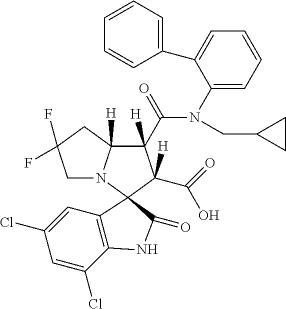 [SMILES: O=C(O)[C@H]1[C@@H](C(=O)N(CC2CC2)c2ccccc2-c2ccccc2)[C@H]2CC(F)(F)CN2[C@]12C(=O)Nc1c(Cl)cc(Cl)cc12] |
| 181 | 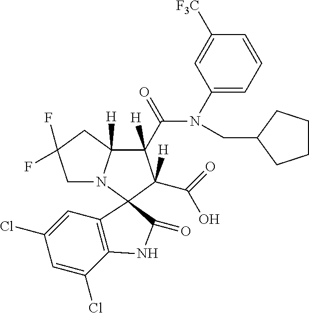 [SMILES: O=C(O)[C@H]1[C@@H](C(=O)N(CC2CCCC2)c2cccc(C(F)(F)F)c2)[C@H]2CC(F)(F)CN2[C@]12C(=O)Nc1c(Cl)cc(Cl)cc12] |
| 182 | 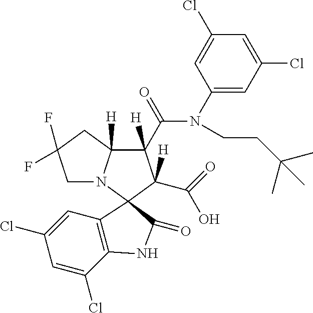 [SMILES: CC(C)(C)CCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 183 | 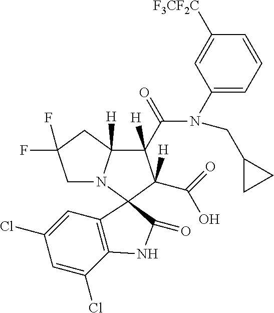 |
| 184 | 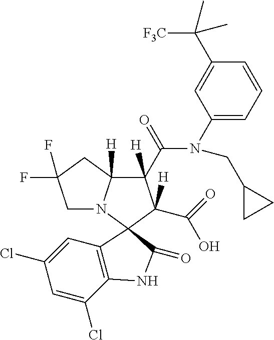 [SMILES: CC(C)(c1cccc(N(CC2CC2)C(=O)[C@H]2[C@H]3CC(F)(F)CN3[C@]3(C(=O)Nc4c(Cl)cc(Cl)cc43)[C@H]2C(=O)O)c1)C(F)(F)F] |
| 185 | 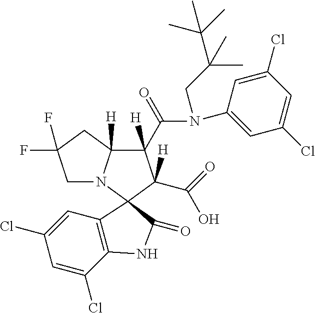 [SMILES: CC(C)(C)C(C)(C)CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 186 | 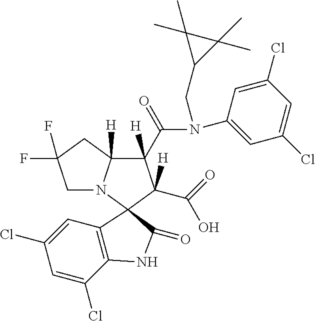 [SMILES: CC1(C)C(CN(C(=O)[C@H]2[C@H]3CC(F)(F)CN3[C@]3(C(=O)Nc4c(Cl)cc(Cl)cc43)[C@H]2C(=O)O)c2cc(Cl)cc(Cl)c2)C1(C)C] |
| 187 | 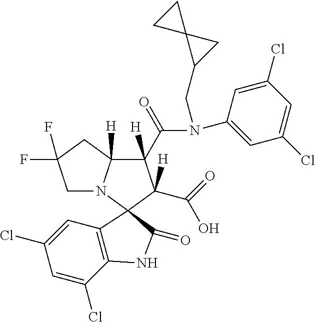 [SMILES: O=C(O)[C@H]1[C@@H](C(=O)N(CC2CC23CC3)c2cc(Cl)cc(Cl)c2)[C@H]2CC(F)(F)CN2[C@]12C(=O)Nc1c(Cl)cc(Cl)cc12] |
| 188 | 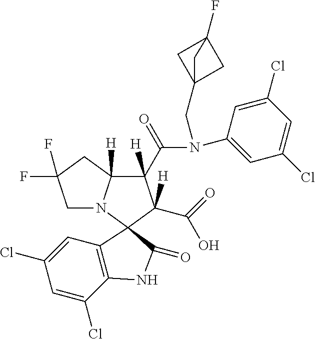 [SMILES: O=C(O)[C@H]1[C@@H](C(=O)N(CC23CC(F)(C2)C3)c2cc(Cl)cc(Cl)c2)[C@H]2CC(F)(F)CN2[C@]12C(=O)Nc1c(Cl)cc(Cl)cc12] |
| 189 | 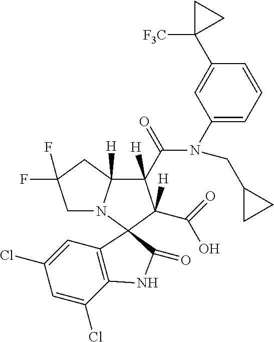 [SMILES: O=C(O)[C@H]1[C@@H](C(=O)N(CC2CC2)c2cccc(C3(C(F)(F)F)CC3)c2)[C@H]2CC(F)(F)CN2[C@]12C(=O)Nc1c(Cl)cc(Cl)cc12] |
| 190 | 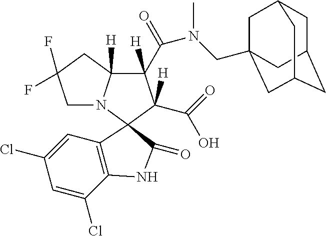 [SMILES: CN(CC12CC3CC(CC(C3)C1)C2)C(=O)[C@H]1[C@H]2CC(F)(F)CN2[C@]2(C(=O)Nc3c(Cl)cc(Cl)cc32)[C@H]1C(=O)O] |
| 191 | 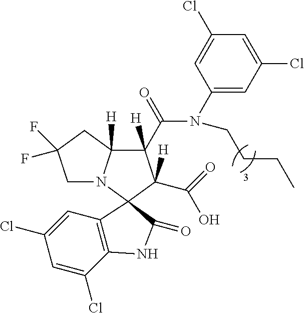 |
| 192 | 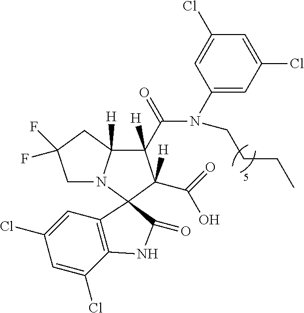 |
| 193 | 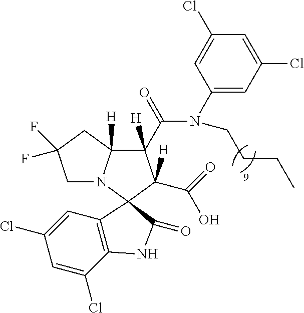 |
| 194 | 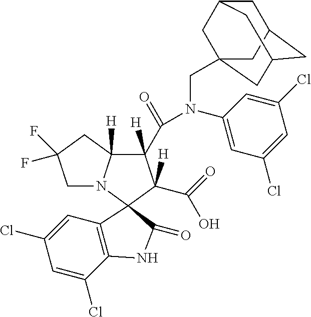 [SMILES: O=C(O)[C@H]1[C@@H](C(=O)N(CC23CC4CC(CC(C4)C2)C3)c2cc(Cl)cc(Cl)c2)[C@H]2CC(F)(F)CN2[C@]12C(=O)Nc1c(Cl)cc(Cl)cc12] |
| 195 | 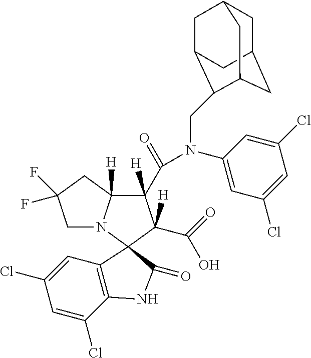 [SMILES: O=C(O)[C@H]1[C@@H](C(=O)N(CC2C3CC4CC(C3)CC2C4)c2cc(Cl)cc(Cl)c2)[C@H]2CC(F)(F)CN2[C@]12C(=O)Nc1c(Cl)cc(Cl)cc12] |
| 196 | 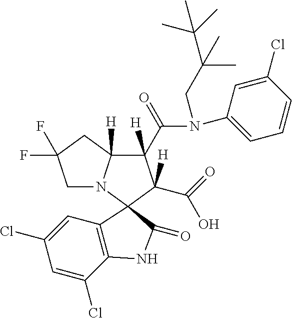 [SMILES: CC(C)(C)C(C)(C)CN(C(=O)[C@H]1[C@H]2CC(F)(F)CN2[C@]2(C(=O)Nc3c(Cl)cc(Cl)cc32)[C@H]1C(=O)O)c1cccc(Cl)c1] |
| 197 | 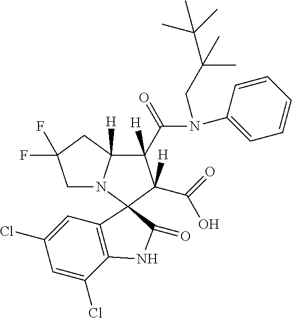 [SMILES: CC(C)(C)C(C)(C)CN(C(=O)[C@H]1[C@H]2CC(F)(F)CN2[C@]2(C(=O)Nc3c(Cl)cc(Cl)cc32)[C@H]1C(=O)O)c1ccccc1] |
| 198 | 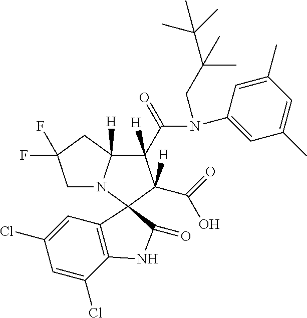 [SMILES: Cc1cc(C)cc(N(CC(C)(C)C(C)(C)C)C(=O)[C@H]2[C@H]3CC(F)(F)CN3[C@]3(C(=O)Nc4c(Cl)cc(Cl)cc43)[C@H]2C(=O)O)c1] |
| 199 | 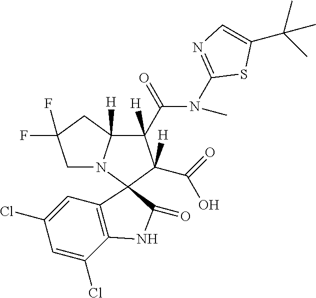 [SMILES: CN(C(=O)[C@H]1[C@H]2CC(F)(F)CN2[C@]2(C(=O)Nc3c(Cl)cc(Cl)cc32)[C@H]1C(=O)O)c1ncc(C(C)(C)C)s1] |
| 200 | 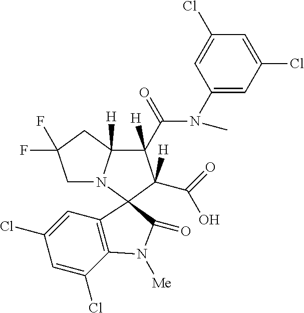 [SMILES: CN(C(=O)[C@H]1[C@H]2CC(F)(F)CN2[C@]2(C(=O)N(C)c3c(Cl)cc(Cl)cc32)[C@H]1C(=O)O)c1cc(Cl)cc(Cl)c1] |
| 201 | 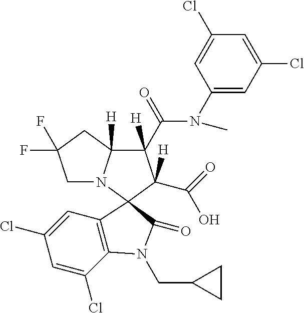 [SMILES: CN(C(=O)[C@H]1[C@H]2CC(F)(F)CN2[C@]2(C(=O)N(CC3CC3)c3c(Cl)cc(Cl)cc32)[C@H]1C(=O)O)c1cc(Cl)cc(Cl)c1] |
| 202 | 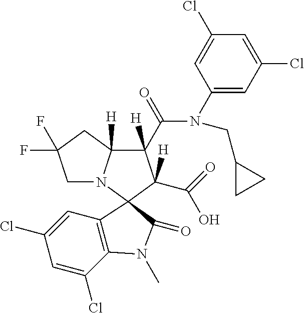 [SMILES: CN1C(=O)[C@]2(c3cc(Cl)cc(Cl)c31)[C@@H](C(=O)O)[C@@H](C(=O)N(CC1CC1)c1cc(Cl)cc(Cl)c1)[C@H]1CC(F)(F)CN12] |
| 203 | 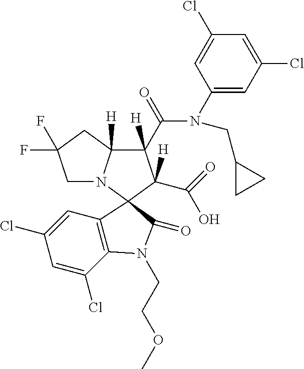 [SMILES: COCCN1C(=O)[C@]2(c3cc(Cl)cc(Cl)c31)[C@@H](C(=O)O)[C@@H](C(=O)N(CC1CC1)c1cc(Cl)cc(Cl)c1)[C@H]1CC(F)(F)CN12] |
| 204 | 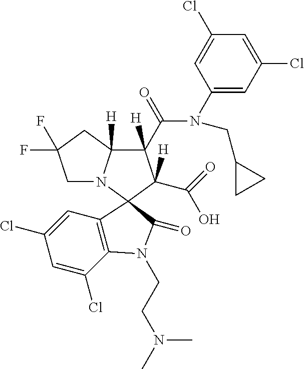 [SMILES: CN(C)CCN1C(=O)[C@]2(c3cc(Cl)cc(Cl)c31)[C@@H](C(=O)O)[C@@H](C(=O)N(CC1CC1)c1cc(Cl)cc(Cl)c1)[C@H]1CC(F)(F)CN12] |
| 205 | 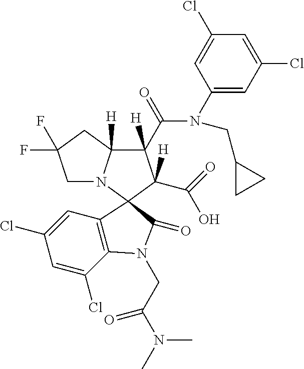 [SMILES: CN(C)C(=O)CN1C(=O)[C@]2(c3cc(Cl)cc(Cl)c31)[C@@H](C(=O)O)[C@@H](C(=O)N(CC1CC1)c1cc(Cl)cc(Cl)c1)[C@H]1CC(F)(F)CN12] |
| 206 | 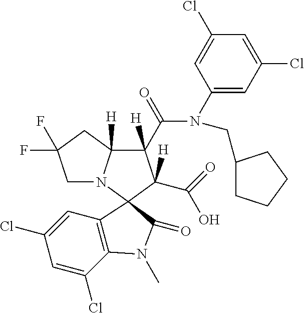 [SMILES: CN1C(=O)[C@]2(c3cc(Cl)cc(Cl)c31)[C@@H](C(=O)O)[C@@H](C(=O)N(CC1CCCC1)c1cc(Cl)cc(Cl)c1)[C@H]1CC(F)(F)CN12] |
| 207 | 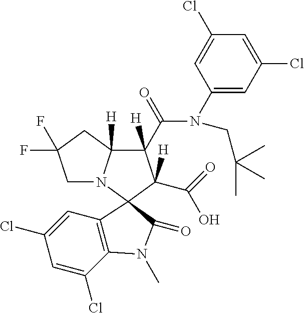 [SMILES: CN1C(=O)[C@]2(c3cc(Cl)cc(Cl)c31)[C@@H](C(=O)O)[C@@H](C(=O)N(CC(C)(C)C)c1cc(Cl)cc(Cl)c1)[C@H]1CC(F)(F)CN12] |
| 208 | 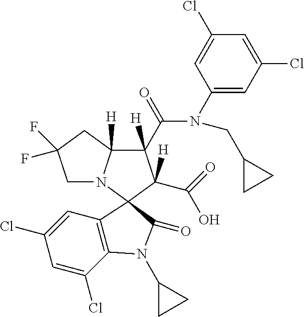 [SMILES: O=C(O)[C@H]1[C@@H](C(=O)N(CC2CC2)c2cc(Cl)cc(Cl)c2)[C@H]2CC(F)(F)CN2[C@]12C(=O)N(C1CC1)c1c(Cl)cc(Cl)cc12] |
| 216.8a | 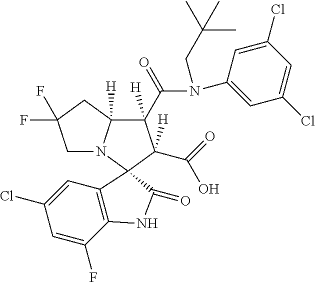 [SMILES: CC(C)(C)CN(C(=O)[C@H]1[C@@H](C(=O)O)[C@]2(C(=O)Nc3c(F)cc(Cl)cc32)N2CC(F)(F)C[C@@H]12)c1cc(Cl)cc(Cl)c1] |
| 216.8b | 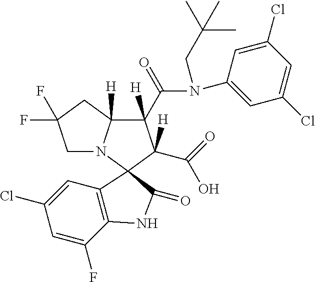 [SMILES: CC(C)(C)CN(C(=O)[C@H]1[C@H]2CC(F)(F)CN2[C@]2(C(=O)Nc3c(F)cc(Cl)cc32)[C@H]1C(=O)O)c1cc(Cl)cc(Cl)c1] |
| 217a | 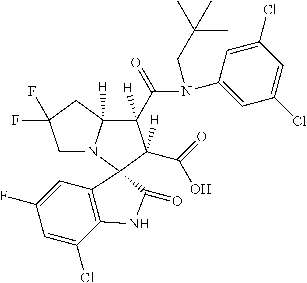 [SMILES: CC(C)(C)CN(C(=O)[C@H]1[C@@H](C(=O)O)[C@]2(C(=O)Nc3c(Cl)cc(F)cc32)N2CC(F)(F)C[C@@H]12)c1cc(Cl)cc(Cl)c1] |
| 217b | 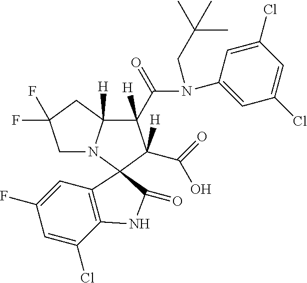 [SMILES: CC(C)(C)CN(C(=O)[C@H]1[C@H]2CC(F)(F)CN2[C@]2(C(=O)Nc3c(Cl)cc(F)cc32)[C@H]1C(=O)O)c1cc(Cl)cc(Cl)c1] |
| 218a | 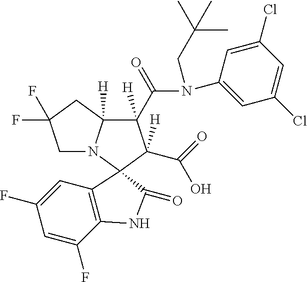 [SMILES: CC(C)(C)CN(C(=O)[C@H]1[C@@H](C(=O)O)[C@]2(C(=O)Nc3c(F)cc(F)cc32)N2CC(F)(F)C[C@@H]12)c1cc(Cl)cc(Cl)c1] |
| 218b | 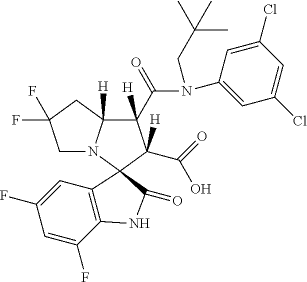 [SMILES: CC(C)(C)CN(C(=O)[C@H]1[C@H]2CC(F)(F)CN2[C@]2(C(=O)Nc3c(F)cc(F)cc32)[C@H]1C(=O)O)c1cc(Cl)cc(Cl)c1] |
| 219 | 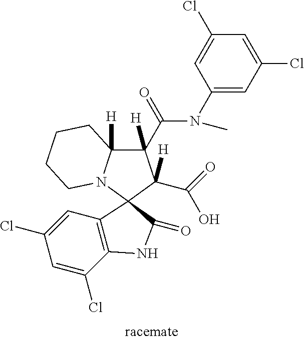 [SMILES: CN(C(=O)[C@H]1[C@H]2CCCCN2[C@]2(C(=O)Nc3c(Cl)cc(Cl)cc32)[C@H]1C(=O)O)c1cc(Cl)cc(Cl)c1] |
| 220.7a 220.7c | 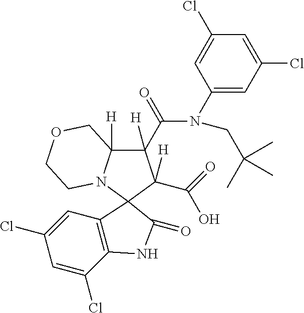 [SMILES: CC(C)(C)CN(C(=O)C1C2COCCN2C2(C(=O)Nc3c(Cl)cc(Cl)cc32)C1C(=O)O)c1cc(Cl)cc(Cl)c1] |
| 225a 225b | 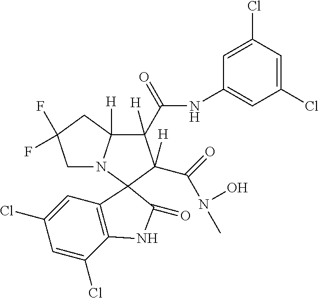 [SMILES: CN(O)C(=O)C1C(C(=O)Nc2cc(Cl)cc(Cl)c2)C2CC(F)(F)CN2C12C(=O)Nc1c(Cl)cc(Cl)cc12] |
| 226a 226b | 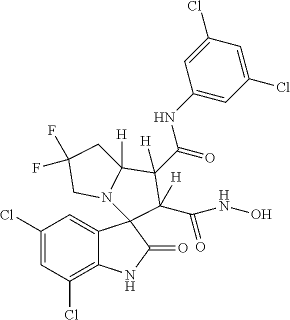 [SMILES: O=C(Nc1cc(Cl)cc(Cl)c1)C1C2CC(F)(F)CN2C2(C(=O)Nc3c(Cl)cc(Cl)cc32)C1C(=O)NO] |
| 227 | 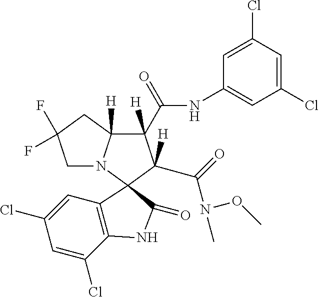 [SMILES: CON(C)C(=O)[C@H]1[C@@H](C(=O)Nc2cc(Cl)cc(Cl)c2)[C@H]2CC(F)(F)CN2[C@]12C(=O)Nc1c(Cl)cc(Cl)cc12] |
| 228 | 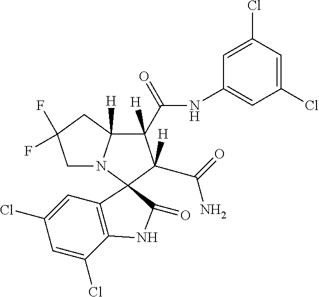 [SMILES: NC(=O)[C@H]1[C@@H](C(=O)Nc2cc(Cl)cc(Cl)c2)[C@H]2CC(F)(F)CN2[C@]12C(=O)Nc1c(Cl)cc(Cl)cc12] |
| 229a 229b | 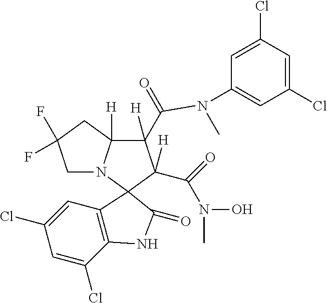 [SMILES: CN(O)C(=O)C1C(C(=O)N(C)c2cc(Cl)cc(Cl)c2)C2CC(F)(F)CN2C12C(=O)Nc1c(Cl)cc(Cl)cc12] |
| 230 | 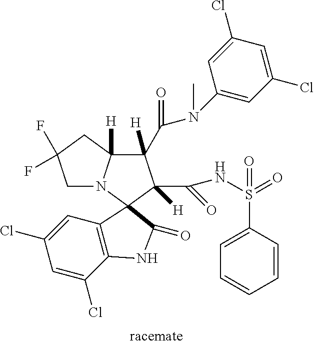 |
| 231 | 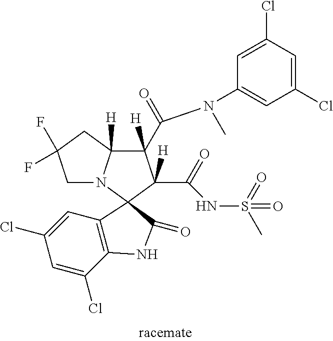 [SMILES: CN(C(=O)[C@H]1[C@H]2CC(F)(F)CN2[C@]2(C(=O)Nc3c(Cl)cc(Cl)cc32)[C@H]1C(=O)NS(C)(=O)=O)c1cc(Cl)cc(Cl)c1] |
| 232a | 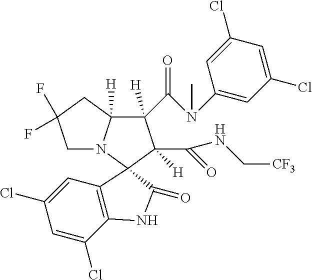 [SMILES: CN(C(=O)[C@H]1[C@@H](C(=O)NCC(F)(F)F)[C@]2(C(=O)Nc3c(Cl)cc(Cl)cc32)N2CC(F)(F)C[C@@H]12)c1cc(Cl)cc(Cl)c1] |
| 232b | 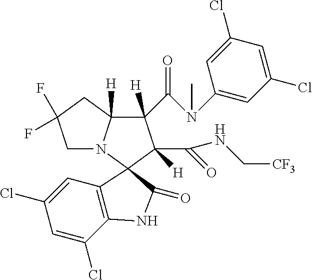 [SMILES: CN(C(=O)[C@H]1[C@H]2CC(F)(F)CN2[C@]2(C(=O)Nc3c(Cl)cc(Cl)cc32)[C@H]1C(=O)NCC(F)(F)F)c1cc(Cl)cc(Cl)c1] |
| 233 | 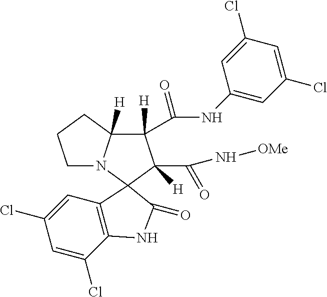 [SMILES: CONC(=O)[C@H]1[C@@H](C(=O)Nc2cc(Cl)cc(Cl)c2)[C@H]2CCCN2C12C(=O)Nc1c(Cl)cc(Cl)cc12] |
| 234a 234b | 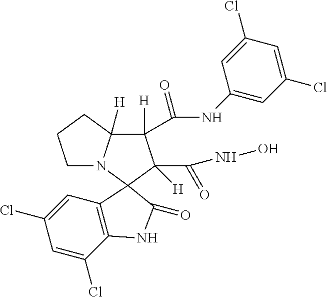 [SMILES: O=C(Nc1cc(Cl)cc(Cl)c1)C1C2CCCN2C2(C(=O)Nc3c(Cl)cc(Cl)cc32)C1C(=O)NO] |
| 235a 235b | 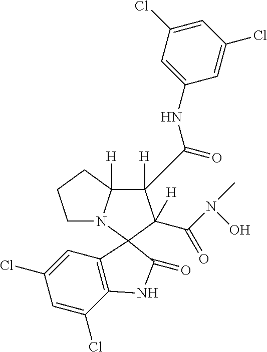 [SMILES: CN(O)C(=O)C1C(C(=O)Nc2cc(Cl)cc(Cl)c2)C2CCCN2C12C(=O)Nc1c(Cl)cc(Cl)cc12] |
| 236 | 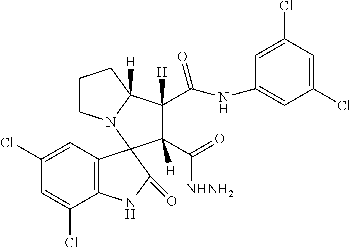 [SMILES: NNC(=O)[C@H]1[C@@H](C(=O)Nc2cc(Cl)cc(Cl)c2)[C@H]2CCCN2C12C(=O)Nc1c(Cl)cc(Cl)cc12] |
| 237 | 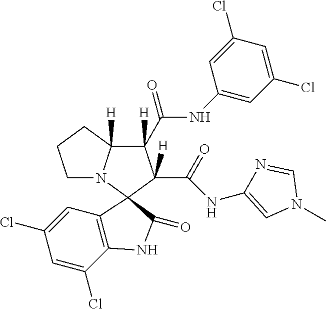 [SMILES: Cn1cnc(NC(=O)[C@H]2[C@@H](C(=O)Nc3cc(Cl)cc(Cl)c3)[C@H]3CCCN3[C@]23C(=O)Nc2c(Cl)cc(Cl)cc23)c1] |
| 238 | 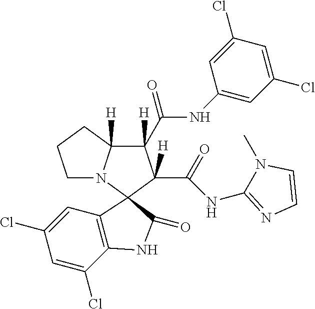 [SMILES: Cn1ccnc1NC(=O)[C@H]1[C@@H](C(=O)Nc2cc(Cl)cc(Cl)c2)[C@H]2CCCN2[C@]12C(=O)Nc1c(Cl)cc(Cl)cc12] |
| 239 | 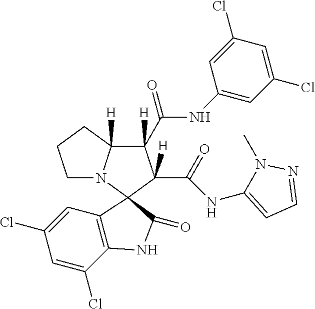 [SMILES: Cn1nccc1NC(=O)[C@H]1[C@@H](C(=O)Nc2cc(Cl)cc(Cl)c2)[C@H]2CCCN2[C@]12C(=O)Nc1c(Cl)cc(Cl)cc12] |
| 240 | 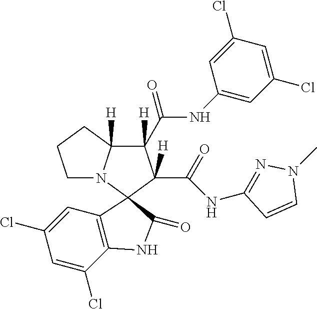 [SMILES: Cn1ccc(NC(=O)[C@H]2[C@@H](C(=O)Nc3cc(Cl)cc(Cl)c3)[C@H]3CCCN3[C@]23C(=O)Nc2c(Cl)cc(Cl)cc23)n1] |
| 241 | 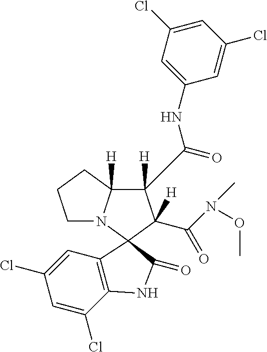 [SMILES: CON(C)C(=O)[C@H]1[C@@H](C(=O)Nc2cc(Cl)cc(Cl)c2)[C@H]2CCCN2[C@]12C(=O)Nc1c(Cl)cc(Cl)cc12] |
| 242 | 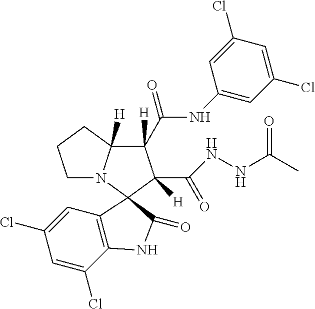 [SMILES: CC(=O)NNC(=O)[C@H]1[C@@H](C(=O)Nc2cc(Cl)cc(Cl)c2)[C@H]2CCCN2[C@]12C(=O)Nc1c(Cl)cc(Cl)cc12] |
| 243 | 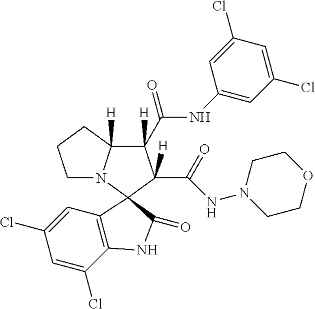 [SMILES: O=C(Nc1cc(Cl)cc(Cl)c1)[C@H]1[C@H]2CCCN2[C@]2(C(=O)Nc3c(Cl)cc(Cl)cc32)[C@H]1C(=O)NN1CCOCC1] |
| 244 | 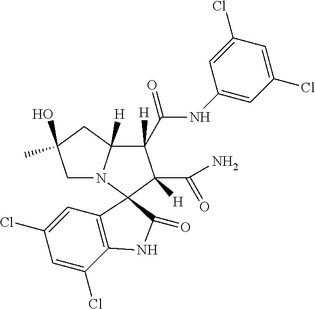 [SMILES: C[C@]1(O)C[C@@H]2[C@H](C(=O)Nc3cc(Cl)cc(Cl)c3)[C@H](C(N)=O)[C@@]3(C(=O)Nc4c(Cl)cc(Cl)cc43)N2C1] |
| 245 | 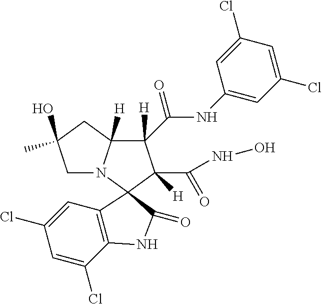 [SMILES: C[C@]1(O)C[C@@H]2[C@H](C(=O)Nc3cc(Cl)cc(Cl)c3)[C@H](C(=O)NO)[C@@]3(C(=O)Nc4c(Cl)cc(Cl)cc43)N2C1] |
| 246 | 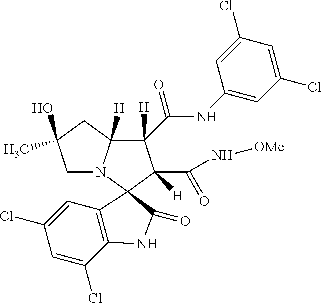 [SMILES: CONC(=O)[C@H]1[C@@H](C(=O)Nc2cc(Cl)cc(Cl)c2)[C@H]2C[C@](C)(O)CN2[C@]12C(=O)Nc1c(Cl)cc(Cl)cc12] |
| 247 | 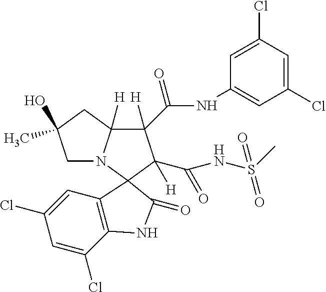 [SMILES: C[C@]1(O)CC2C(C(=O)Nc3cc(Cl)cc(Cl)c3)C(C(=O)NS(C)(=O)=O)C3(C(=O)Nc4c(Cl)cc(Cl)cc43)N2C1] |
| 248 | 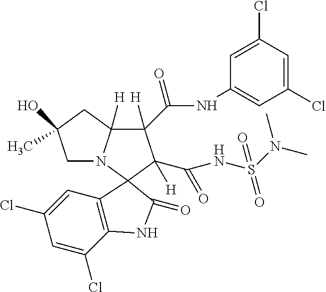 [SMILES: CN(C)S(=O)(=O)NC(=O)C1C(C(=O)Nc2cc(Cl)cc(Cl)c2)C2C[C@](C)(O)CN2C12C(=O)Nc1c(Cl)cc(Cl)cc12] |
| 249a | 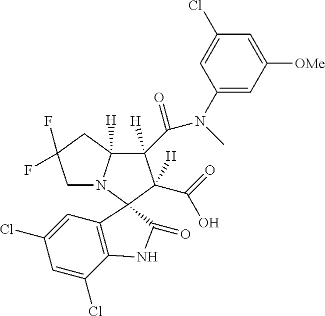 [SMILES: COc1cc(Cl)cc(N(C)C(=O)[C@H]2[C@@H](C(=O)O)[C@]3(C(=O)Nc4c(Cl)cc(Cl)cc43)N3CC(F)(F)C[C@@H]23)c1] |
| 249b | 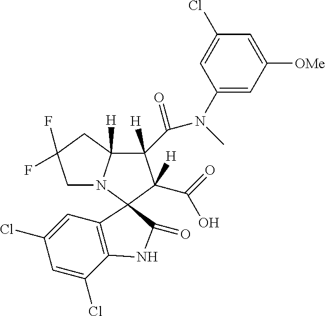 [SMILES: COc1cc(Cl)cc(N(C)C(=O)[C@H]2[C@H]3CC(F)(F)CN3[C@]3(C(=O)Nc4c(Cl)cc(Cl)cc43)[C@H]2C(=O)O)c1] |
| 250.3a | 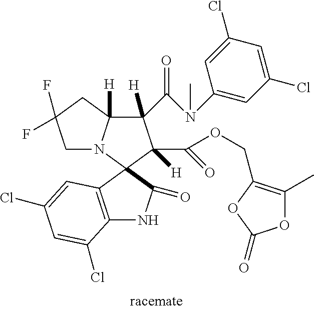 |
| 250.3b | 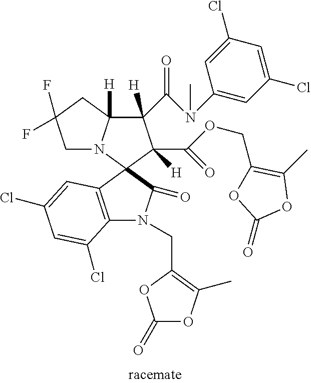 |
| 251a | 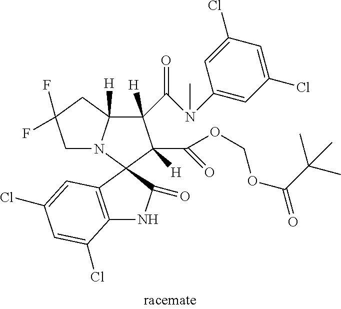 [SMILES: CN(C(=O)[C@H]1[C@H]2CC(F)(F)CN2[C@]2(C(=O)Nc3c(Cl)cc(Cl)cc32)[C@H]1C(=O)OCOC(=O)C(C)(C)C)c1cc(Cl)cc(Cl)c1] |
| 251c | 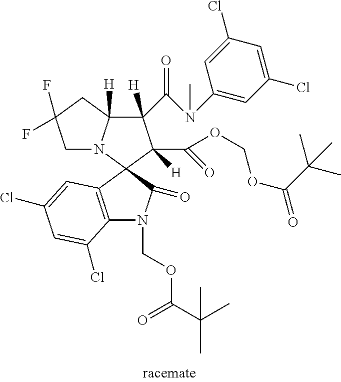 [SMILES: CN(C(=O)[C@H]1[C@H]2CC(F)(F)CN2[C@]2(C(=O)N(COC(=O)C(C)(C)C)c3c(Cl)cc(Cl)cc32)[C@H]1C(=O)OCOC(=O)C(C)(C)C)c1cc(Cl)cc(Cl)c1] |
| 252 | 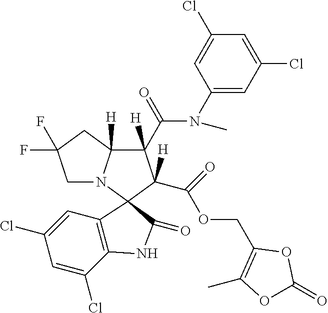 [SMILES: Cc1oc(=O)oc1COC(=O)[C@H]1[C@@H](C(=O)N(C)c2cc(Cl)cc(Cl)c2)[C@H]2CC(F)(F)CN2[C@]12C(=O)Nc1c(Cl)cc(Cl)cc12] |
| 253 | 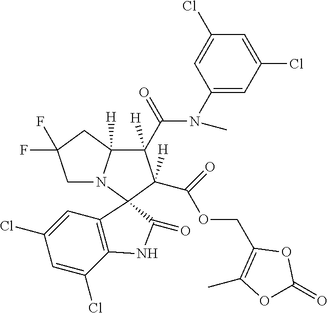 [SMILES: Cc1oc(=O)oc1COC(=O)[C@@H]1[C@H](C(=O)N(C)c2cc(Cl)cc(Cl)c2)[C@@H]2CC(F)(F)CN2[C@@]12C(=O)Nc1c(Cl)cc(Cl)cc12] |
| 254a | 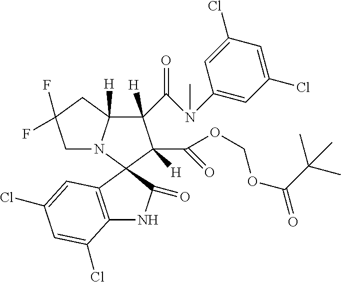 [SMILES: CN(C(=O)[C@H]1[C@H]2CC(F)(F)CN2[C@]2(C(=O)Nc3c(Cl)cc(Cl)cc32)[C@H]1C(=O)OCOC(=O)C(C)(C)C)c1cc(Cl)cc(Cl)c1] |
| 254b | 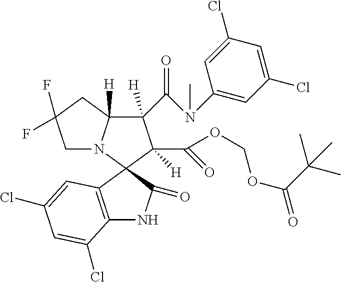 [SMILES: CN(C(=O)[C@@H]1[C@H]2CC(F)(F)CN2[C@]2(C(=O)Nc3c(Cl)cc(Cl)cc32)[C@@H]1C(=O)OCOC(=O)C(C)(C)C)c1cc(Cl)cc(Cl)c1] |
| 255a | 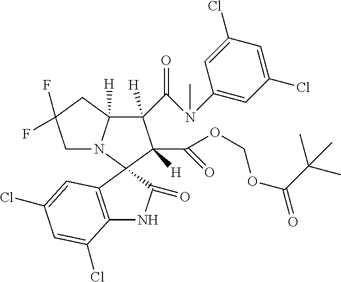 [SMILES: CN(C(=O)[C@@H]1[C@@H]2CC(F)(F)CN2[C@@]2(C(=O)Nc3c(Cl)cc(Cl)cc32)[C@H]1C(=O)OCOC(=O)C(C)(C)C)c1cc(Cl)cc(Cl)c1] |
| 255b | 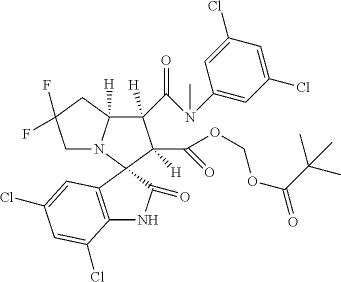 [SMILES: CN(C(=O)[C@H]1[C@@H](C(=O)OCOC(=O)C(C)(C)C)[C@]2(C(=O)Nc3c(Cl)cc(Cl)cc32)N2CC(F)(F)C[C@@H]12)c1cc(Cl)cc(Cl)c1] |
| 256a | 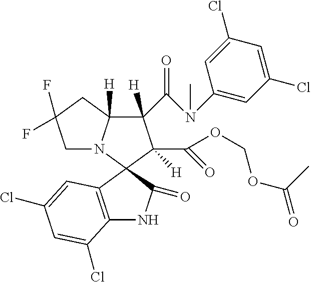 [SMILES: CC(=O)OCOC(=O)[C@@H]1[C@@H](C(=O)N(C)c2cc(Cl)cc(Cl)c2)[C@H]2CC(F)(F)CN2[C@]12C(=O)Nc1c(Cl)cc(Cl)cc12] |
| 256b | 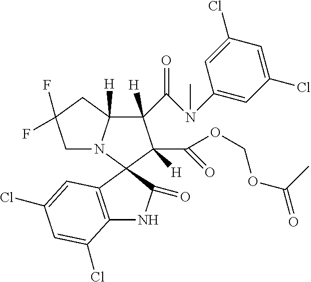 [SMILES: CC(=O)OCOC(=O)[C@H]1[C@@H](C(=O)N(C)c2cc(Cl)cc(Cl)c2)[C@H]2CC(F)(F)CN2[C@]12C(=O)Nc1c(Cl)cc(Cl)cc12] |
| 257 | 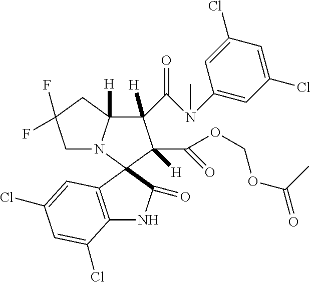 |
| 258 | 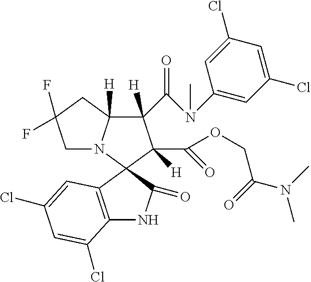 [SMILES: CN(C)C(=O)COC(=O)[C@H]1[C@@H](C(=O)N(C)c2cc(Cl)cc(Cl)c2)[C@H]2CC(F)(F)CN2[C@]12C(=O)Nc1c(Cl)cc(Cl)cc12] |
| 259 | 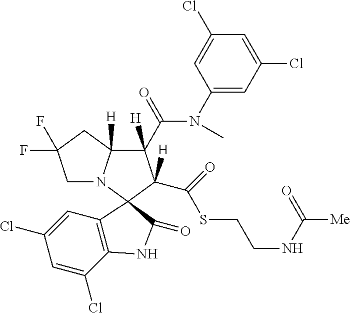 [SMILES: CC(=O)NCCSC(=O)[C@H]1[C@@H](C(=O)N(C)c2cc(Cl)cc(Cl)c2)[C@H]2CC(F)(F)CN2[C@]12C(=O)Nc1c(Cl)cc(Cl)cc12] |
| 260a | 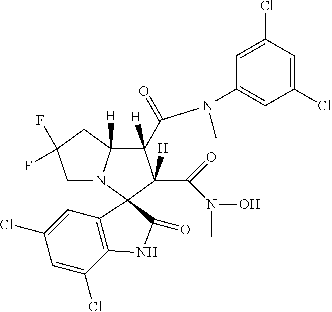 [SMILES: CN(O)C(=O)[C@H]1[C@@H](C(=O)N(C)c2cc(Cl)cc(Cl)c2)[C@H]2CC(F)(F)CN2[C@]12C(=O)Nc1c(Cl)cc(Cl)cc12] |
| 260b | 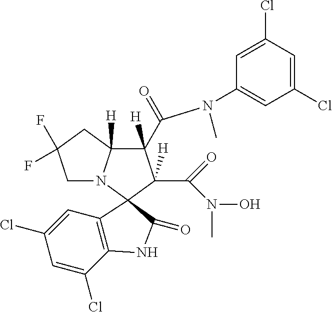 [SMILES: CN(O)C(=O)[C@@H]1[C@@H](C(=O)N(C)c2cc(Cl)cc(Cl)c2)[C@H]2CC(F)(F)CN2[C@]12C(=O)Nc1c(Cl)cc(Cl)cc12] |
| 261 | 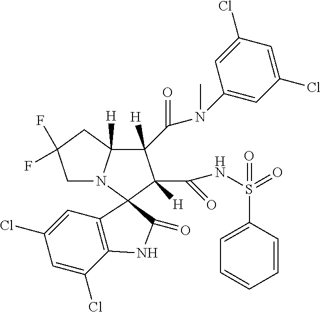 [SMILES: CN(C(=O)[C@H]1[C@H]2CC(F)(F)CN2[C@]2(C(=O)Nc3c(Cl)cc(Cl)cc32)[C@H]1C(=O)NS(=O)(=O)c1ccccc1)c1cc(Cl)cc(Cl)c1] |
| 265a | 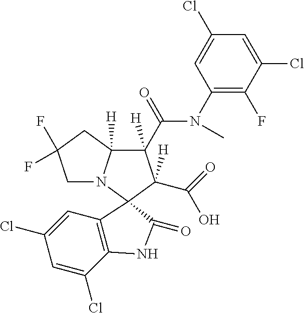 [SMILES: CN(C(=O)[C@H]1[C@@H](C(=O)O)[C@]2(C(=O)Nc3c(Cl)cc(Cl)cc32)N2CC(F)(F)C[C@@H]12)c1cc(Cl)cc(Cl)c1F] |
| 265b | 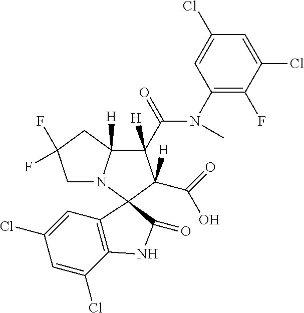 [SMILES: CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1F] |
| 266 | 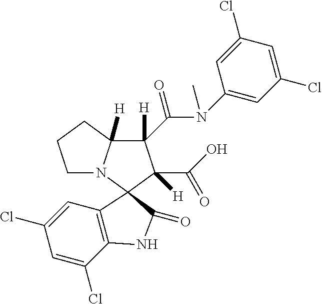 [SMILES: CN(C(=O)[C@H]1[C@H]2CCCN2[C@]2(C(=O)Nc3c(Cl)cc(Cl)cc32)[C@H]1C(=O)O)c1cc(Cl)cc(Cl)c1] |
| 267 | 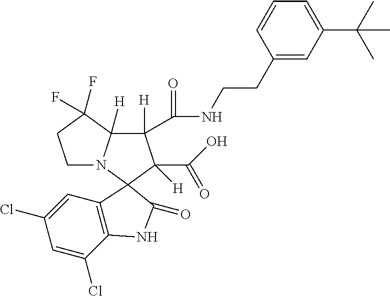 [SMILES: CC(C)(C)c1cccc(CCNC(=O)C2C3N(CCC3(F)F)C3(C(=O)Nc4c(Cl)cc(Cl)cc43)C2C(=O)O)c1] |
| 268a 268b | 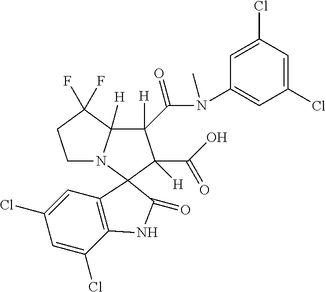 [SMILES: CN(C(=O)C1C2N(CCC2(F)F)C2(C(=O)Nc3c(Cl)cc(Cl)cc32)C1C(=O)O)c1cc(Cl)cc(Cl)c1] |
| 269a | 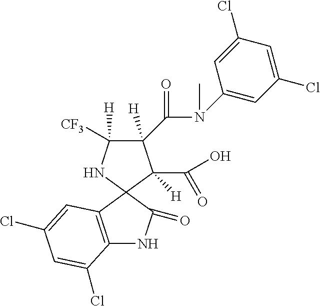 [SMILES: CN(C(=O)[C@@H]1[C@H](C(F)(F)F)NC2(C(=O)Nc3c(Cl)cc(Cl)cc32)[C@@H]1C(=O)O)c1cc(Cl)cc(Cl)c1] |
| 269b | 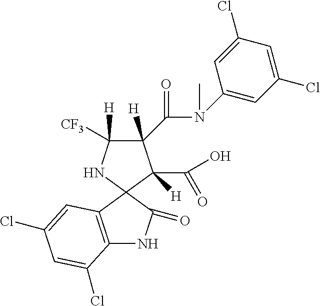 [SMILES: CN(C(=O)[C@H]1[C@@H](C(F)(F)F)NC2(C(=O)Nc3c(Cl)cc(Cl)cc32)[C@H]1C(=O)O)c1cc(Cl)cc(Cl)c1] |
| 270a | 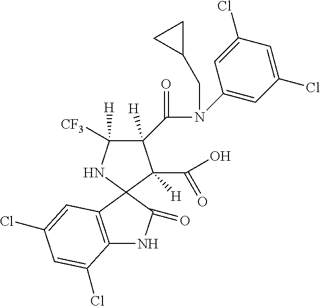 [SMILES: O=C(O)[C@@H]1[C@H](C(=O)N(CC2CC2)c2cc(Cl)cc(Cl)c2)[C@H](C(F)(F)F)NC12C(=O)Nc1c(Cl)cc(Cl)cc12] |
| 270b | 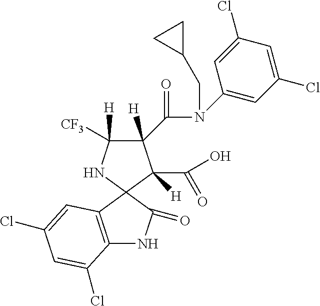 [SMILES: O=C(O)[C@H]1[C@@H](C(=O)N(CC2CC2)c2cc(Cl)cc(Cl)c2)[C@@H](C(F)(F)F)NC12C(=O)Nc1c(Cl)cc(Cl)cc12] |
| 271 | 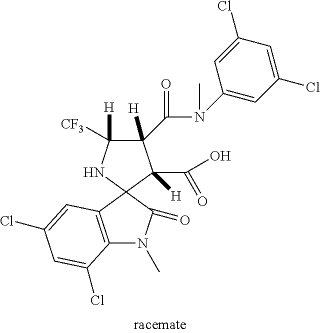 [SMILES: CN(C(=O)[C@H]1[C@@H](C(F)(F)F)NC2(C(=O)N(C)c3c(Cl)cc(Cl)cc32)[C@H]1C(=O)O)c1cc(Cl)cc(Cl)c1] |
| 272 | 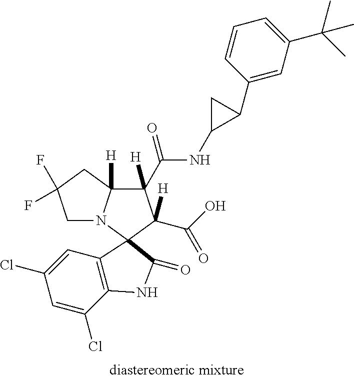 |
| 273a 273b | 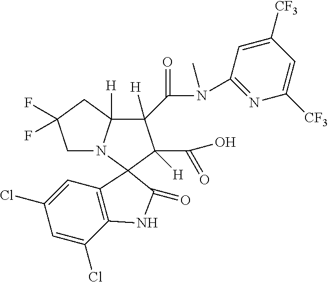 [SMILES: CN(C(=O)C1C2CC(F)(F)CN2C2(C(=O)Nc3c(Cl)cc(Cl)cc32)C1C(=O)O)c1cc(C(F)(F)F)cc(C(F)(F)F)n1] |
| 274.6a | 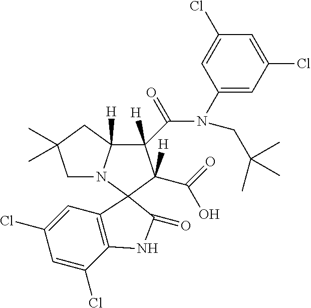 [SMILES: CC(C)(C)CN(C(=O)[C@H]1[C@H]2CC(C)(C)CN2C2(C(=O)Nc3c(Cl)cc(Cl)cc32)[C@H]1C(=O)O)c1cc(Cl)cc(Cl)c1] |
| 274.6b | 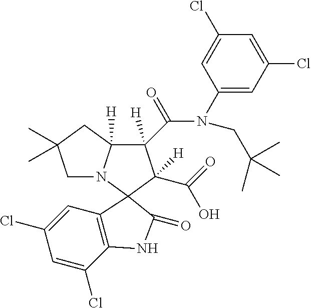 [SMILES: CC(C)(C)CN(C(=O)[C@H]1[C@@H](C(=O)O)C2(C(=O)Nc3c(Cl)cc(Cl)cc32)N2CC(C)(C)C[C@@H]12)c1cc(Cl)cc(Cl)c1] |
| 275.7a | 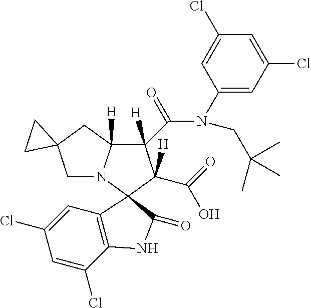 [SMILES: CC(C)(C)CN(C(=O)[C@H]1[C@H]2CC3(CC3)CN2[C@]2(C(=O)Nc3c(Cl)cc(Cl)cc32)[C@H]1C(=O)O)c1cc(Cl)cc(Cl)c1] |
| 275.7b | 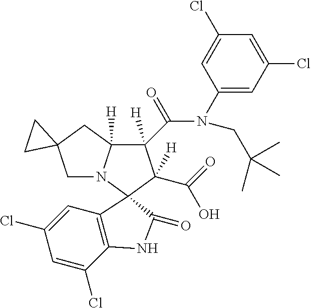 [SMILES: CC(C)(C)CN(C(=O)[C@H]1[C@@H](C(=O)O)[C@]2(C(=O)Nc3c(Cl)cc(Cl)cc32)N2CC3(CC3)C[C@@H]12)c1cc(Cl)cc(Cl)c1] |
| 280 | 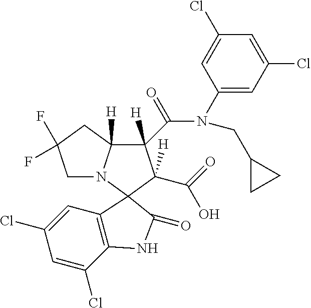 [SMILES: O=C(O)[C@@H]1[C@@H](C(=O)N(CC2CC2)c2cc(Cl)cc(Cl)c2)[C@H]2CC(F)(F)CN2C12C(=O)Nc1c(Cl)cc(Cl)cc12] |
| 281 | 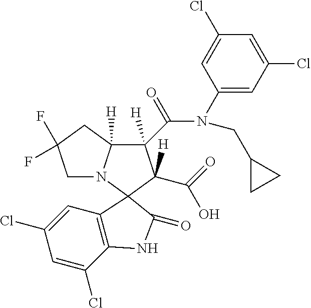 [SMILES: O=C(O)[C@H]1[C@H](C(=O)N(CC2CC2)c2cc(Cl)cc(Cl)c2)[C@@H]2CC(F)(F)CN2C12C(=O)Nc1c(Cl)cc(Cl)cc12] |
| 282 | 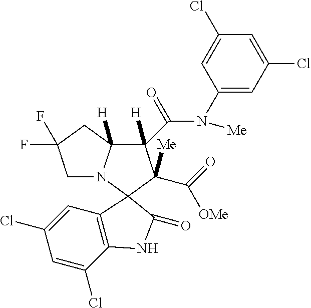 [SMILES: COC(=O)[C@@]1(C)[C@@H](C(=O)N(C)c2cc(Cl)cc(Cl)c2)[C@H]2CC(F)(F)CN2C12C(=O)Nc1c(Cl)cc(Cl)cc12] |
| 284 | 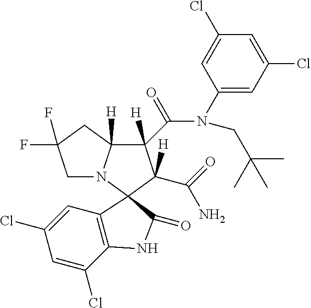 [SMILES: CC(C)(C)CN(C(=O)[C@H]1[C@H]2CC(F)(F)CN2[C@]2(C(=O)Nc3c(Cl)cc(Cl)cc32)[C@H]1C(N)=O)c1cc(Cl)cc(Cl)c1] |
| 285 | 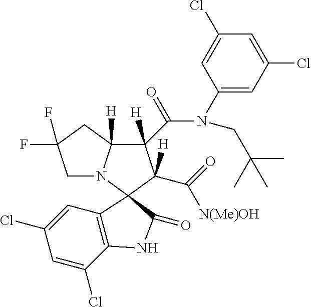 |
| 286a 286b | 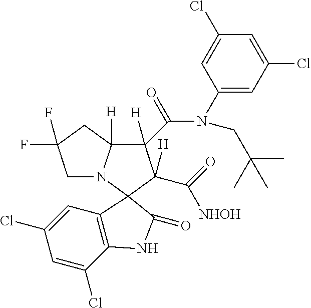 |
| 287 | 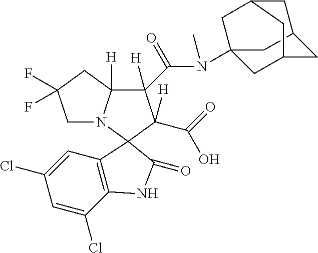 [SMILES: CN(C(=O)C1C2CC(F)(F)CN2C2(C(=O)Nc3c(Cl)cc(Cl)cc32)C1C(=O)O)C12CC3CC(CC(C3)C1)C2] |
| 288a | 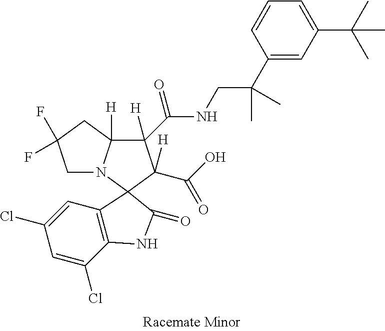 [SMILES: CC(C)(C)c1cccc(C(C)(C)CNC(=O)C2C3CC(F)(F)CN3C3(C(=O)Nc4c(Cl)cc(Cl)cc43)C2C(=O)O)c1] |
| 288b | 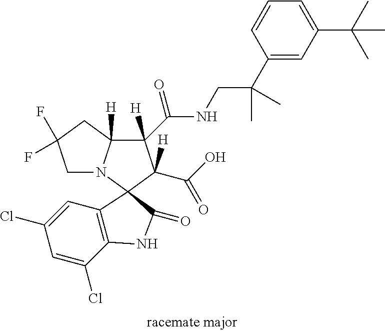 [SMILES: CC(C)(C)c1cccc(C(C)(C)CNC(=O)[C@H]2[C@H]3CC(F)(F)CN3[C@]3(C(=O)Nc4c(Cl)cc(Cl)cc43)[C@H]2C(=O)O)c1] |
| 289 | 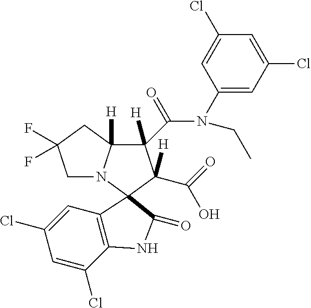 |
| 290 | 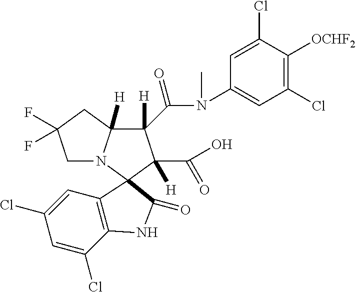 |
| 291 | 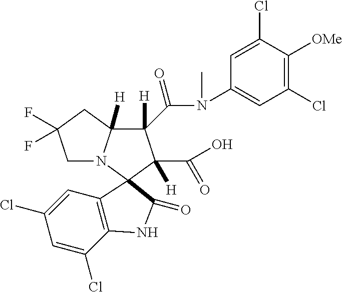 |
| 292 | 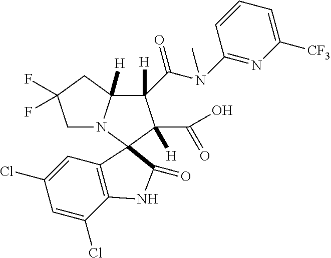 |
| 293 | 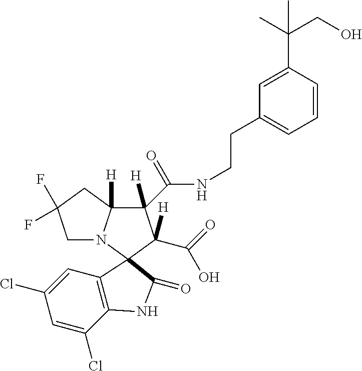 |
| 294 | 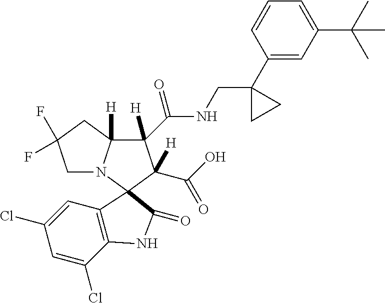 |

In compounds illustrated herein in which the stereochemistry is not indicated, the compound is preferably the stereoisomer having the absolute stereochemistry indicated in Formulas (Ia), (IIa), (IIIa), (IVa) and (Va) or Formulas (Ib), (llb), (IIIb), (IVb) and (Vb). In certain embodiments, the preferred stereoisomer has the absolute stereochemistry indicated in Formulas (Ia), (IIa), (IIIa), (IVa) and (Va).

The compounds of the invention are useful as modulators of CFTR and treating diseases or disorders mediated by CFTR. The present invention, thus, provides methods of treating a disease or disorder mediated by CFTR in a subject in need thereof, comprising administering to the subject a therapeutically effective amount of a compound of the invention. Diseases or disorders mediated by CFTR include cystic fibrosis, Asthma, Constipation, Pancreatitis, Gastrointestinal diseases or disorders, Infertility, Hereditary emphysema, Hereditary hemochromatosis, Coagulation-Fibrinolysis deficiencies, such as Protein C deficiency, Type 1 hereditary angioedema, Lipid processing deficiencies, such as Familial hypercholesterolemia, Type 1 chylomicronemia, Abetalipoproteinemia, Lysosomal storage diseases, such as I-cell disease/Pseudo-Hurler, Mucopolysaccharidoses, Sandhof/Tay-Sachs, Crigler-Najjar type II, Polyendocrinopathy/Hyperinsulemia, Diabetes mellitus, Laron dwarfism, Myeloperoxidase deficiency, Primary hypoparathyroidism, Melanoma, Glycanosis CDG type 1, Hereditary emphysema, Congenital hyperthyroidism, Osteogenesis imperfecta, Hereditary hypofibrinogenemia, ACT deficiency, Diabetes insipidus (DI), Neurophyseal DI, Neprogenic DI, Charcot-Marie Tooth syndrome, Perlizaeus-Merzbacher disease, neurodegenerative diseases such as Alzheimer's disease, Parkinson's disease, Amyotrophic lateral sclerosis, Progressive supranuclear palsy, Pick's disease, several polyglutamine neurological disorders such as Huntington's disease, Spinocerebullar ataxia type I, Spinal and bulbar muscular atrophy, Dentororubal pallidoluysian, and Myotonic dystrophy, as well as spongiform encephalopathies such as Hereditary Creutzfeldt-Jakob disease, Fabry disease, and Straussler-Scheinker disease; secretory diarrhea, polycystic kidney disease, chronic obstructive pulmonary disease (COPD), dry eye disease, Sjogren's Syndrome, congenital bilateral absence of vas deferens (CBAVD), disseminated bronchiectasis, allergic pulmonary aspergillosis, chronic sinusitis, protein C deficiency, A-lipoproteinemia, mild pulmonary disease, lipid processing deficiencies, coagulation fibrinolyis, CFTR-related metabolic syndrome, chronic bronchitis, constipation, pancreatic insufficiency, melanoma, glycanosis CDG type 1, ACT deficiency, allergic pulmonary aspergillosis; celiac disease; vascular inflammation-atherosclerotic disease, increased glucagon production, cholestatic liver disease (e.g. Primary biliary cirrhosis (PBC) and primary sclerosing cholangitis (PSC)).

In certain embodiments, the disease or disorder mediated by CFTR is selected from congenital bilateral absence of vas deferens; acute, recurrent or chronic pancreatitis; disseminated bronchiectasis; asthma; allergic pulmonary aspergillosis; smoking related lung disease (e.g., chronic obstructive pulmonary disease, COPD); dry eye disease; Sjogren's syndrome; chronic sinusitis; cholestatic liver disease, such as primary biliary cirrhosis and primary sclerosing cholangitis; and polycystic kidney disease (autosomal dominant).

In certain embodiments, the disease or disorder mediated by CFTR is selected from celiac disease; vascular inflammation-atherosclerotic disease; dry eye (keratoconjunctivitis sicca) with or without associated autoimmune disease; polycystic kidney disease; cystic fibrosis-related diabetes mellitus; increased glucagon production; non-atopic asthma; non-CF bronchiectasis; and constipation.

The compounds of the invention can be administered in combination with one or more additional therapeutic agents, such as antibiotics, anti-inflammatory medicines, bronchodilators, or mucus-thinning medicines. In particular, antibiotics for the treatment of bacteria mucoid Pseudomonas can be used in combination with compounds of the invention. Inhaled antibiotics such as tobramycin, colistin, and aztreonam can be used in combination with treatment with compounds of the invention. Anti-inflammatory medicines can also be used in combination with compounds of the invention to treat CFTR related diseases. Bronchodilators can be used in combination with compounds of the invention to treat CFTR related diseases. In certain embodiments, the compound of the invention is administered in combination with a second compound which is a CFTR modulator.

In one embodiment, the invention provides a method of treating cystic fibrosis or a symptom thereof, in a subject in need thereof, comprising administering to the subject a therapeutically effective amount of a compound of the invention. The compound of the invention is optionally administered in combination with one or more additional pharmaceutical agents useful for the treatment of cystic fibrosis, such as compounds which are CFTR modulators, for example, compounds which are modulators of CFTR expression, activity and/or function. Suitable additional pharmaceutical agents include, but are not limited to, gentamicin ataluren, ivacaftor (KALYDECO™), lumacaftor, tezacaftor, VX-445 PTI-428, PTI-801, PTI-808, GLPG1837, GLPG2222, GLPG2737, FDL169, and FDL176. In certain embodiments, the compound of the invention is administered in combination with two or more additional CFTR modulators. For example, in one embodiment, a compound of the invention is administered in combination with FDL169 and/or FDL176. In one embodiment, the compound of the invention is administered in combination with both FDL169 and FDL176. In one embodiment, the invention relates to a pharmaceutical composition comprising a compound of the invention and a pharmaceutically acceptable excipient or carrier. The compositions can include one or more compounds of the invention, and a pharmaceutically acceptable carrier, adjuvant or vehicle. In certain embodiments, these compositions further comprise one or more additional therapeutic agents useful for the treatment of CFTR mediated diseases or disorders.

### Pharmaceutical Compositions

The pharmaceutical compositions of the present invention comprise a compound of the present invention formulated together with one or more pharmaceutically acceptable carriers or excipients.

As used herein, the term “pharmaceutically acceptable carrier or excipient” means a non-toxic, inert solid, semi-solid, gel or liquid filler, diluent, encapsulating material or formulation auxiliary of any type. Some examples of materials which can serve as pharmaceutically acceptable carriers are sugars such as lactose, glucose and sucrose; cyclodextrins such as alpha—(α), beta—(β) and gamma—(γ) cyclodextrins; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention can be administered orally, parenterally, by inhalation spray, topically, rectally, nasally, buccally, vaginally or via an implanted reservoir. In a preferred embodiment, administration is oral administration. Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

The pharmaceutical compositions of this invention can contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

In another embodiment, administration is parenteral administration by injection. Injectable preparations, for example, sterile injectable aqueous or oleaginous suspensions, may be formulated according to the known art using suitable dispersing or wetting agents and suspending agents. The sterile injectable preparation may also be a sterile injectable suspension or emulsion, such as INTRALIPID®, LIPOSYN® or OMEGAVEN®, or solution, in a nontoxic parenterally acceptable diluent or solvent, for example, as a solution in 1, 3-butanediol. INTRALIPID® is an intravenous fat emulsion containing 10-30% soybean oil, 1-10% egg yolk phospholipids, 1-10% glycerin and water. LIPOSYN® is also an intravenous fat emulsion containing 2-15% safflower oil, 2-15% soybean oil, 0.5-5% egg phosphatides 1-10% glycerin and water. OMEGAVEN® is an emulsion for infusion containing about 5-25% fish oil, 0.5-10% egg phosphatides, 1-10% glycerin and water. Among the acceptable vehicles and solvents that may be employed are water, Ringer's solution, USP and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics is known in the art (see, for example U.S. Pat. No. 5,767,068 to Van Devanter et al., U.S. Pat. No. 5,508,269 to Smith et al., and WO 98/43650 by Montgomery).

The compositions described herein can be formulated in a unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose. The amount of the active compound in a unit dosage form will vary depending upon, for example, the host treated, and the particular mode of administration. In one embodiment, the unit dosage form can have one of the compounds of the invention as an active ingredient in an amount of about 10 mg, 20 mg, 30 mg, 40 mg, 50 mg, 100 mg, 150 mg, 200 mg, 250 mg, 300 mg, 400 mg, 500 mg, 600 mg, 700 mg, 750 mg, 800 mg, 900 mg, 1000 mg, or 1,250 mg.

In some embodiments, the compounds of the invention can be administered in a dose of at least about 10 mg/day to at least about 1500 mg/day. In some embodiments, the compounds of the invention are administered in a dose of at least about 300 mg (e.g., at least about 450 mg, at least about 500 mg, at least about 750 mg, at least about 1,000 mg, at least about 1250 mg, or at least about 1500 mg).

Dose adjustments can be made for patients with mild, moderate or severe hepatic impairment (Child-Pugh Class A). Furthermore, dosage adjustments can be made for patients taking one or more Cytochrome P450 inhibitors and inducers, in particular CYP3A4, CYP2D6, CYP2C₉, CYP2C₁₉ and CYP2B6 inhibitors and inducers. Dose adjustments can also be made for patients with impaired Cytochrome P450 function such as poor, intermediate, extensive and ultra-rapid metabolizers.

### Definitions

Listed below are definitions of various terms used to describe this invention. These definitions apply to the terms as they are used throughout this specification and claims, unless otherwise limited in specific instances, either individually or as part of a larger group. The term “alkyl” is intended to include both branched and straight chain, substituted or unsubstituted saturated aliphatic hydrocarbon radicals/groups having the specified number of carbons. Preferred alkyl groups comprise about 1 to about 24 carbon atoms (“C₁-C₂₄”). Other preferred alkyl groups comprise at about 1 to about 8 carbon atoms (“C₁-C₈”) such as about 1 to about 6 carbon atoms (“C₁-C₆”), or such as about 1 to about 3 carbon atoms (“C₁-C₃”). Examples of C₁-C₆ alkyl radicals include, but are not limited to, methyl, ethyl, propyl, isopropyl, n-butyl, tent-butyl, n-pentyl, neopentyl and n-hexyl radicals.

The term “alkenyl” refers to linear or branched radicals having at least one carbon-carbon double bond. Such radicals preferably contain from about two to about twenty-four carbon atoms (“C₂-C₂₄”). Other preferred alkenyl radicals are “lower alkenyl” radicals having two to about ten carbon atoms (“C₂-C₁₀”) such as ethenyl, allyl, propenyl, butenyl and 4-methylbutenyl. Preferred lower alkenyl radicals include 2 to about 6 carbon atoms (“C₂-C₆”). The terms “alkenyl”, and “lower alkenyl”, embrace radicals having “cis” and “trans” orientations, or alternatively, “E” and “Z” orientations.

The term “alkynyl” refers to linear or branched radicals having at least one carbon-carbon triple bond. Such radicals preferably contain from about two to about twenty-four carbon atoms (“C₂-C₂₄”). Other preferred alkynyl radicals are “lower alkynyl” radicals having two to about ten carbon atoms such as propargyl, 1-propynyl, 2-propynyl, 1-butyne, 2-butynyl and 1-pentynyl. Preferred lower alkynyl radicals include 2 to about 6 carbon atoms (“C₂-C₆”).

The term “aryl,” as used herein, refers to a mono- or polycyclic carbocyclic ring system comprising at least one aromatic ring, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof.

As used herein, the term “arylalkyl” means a functional group wherein an alkylene chain is attached to an aryl group, e.g., —CH₂CH₂-phenyl. The term “substituted arylalkyl” means an arylalkyl functional group in which the aryl group is substituted. Similarly, the term “heteroarylalkyl” means a functional group wherein an alkylene chain is attached to a heteroaryl group. The term “substituted heteroarylalkyl” means a heteroarylalkyl functional group in which the heteroaryl group is substituted.

As used herein, the term “alkoxy” employed alone or in combination with other terms means, unless otherwise stated, an alkyl group having the designated number of carbon atoms connected to the rest of the molecule via an oxygen atom, such as, for example, methoxy, ethoxy, 1-propoxy, 2-propoxy (isopropoxy) and the higher homologs and isomers. Preferred alkoxy are (C₁-C₃) alkoxy.

The term “cycloalkyl” refers to saturated carbocyclic radicals having three to about twelve carbon atoms (“C₃-C₁₂”). The term “cycloalkyl” embraces saturated carbocyclic radicals having three to about twelve carbon atoms. Examples of such radicals include cyclopropyl, cyclobutyl, cyclopentyl and cyclohexyl.

The term “alkoxy” is intended to refer to an alkyl-O-radical.

The term “cycloalkenyl” refers to partially unsaturated carbocyclic radicals having three to twelve carbon atoms. Cycloalkenyl radicals that are partially unsaturated carbocyclic radicals that contain two double bonds (that may or may not be conjugated) can be called “cycloalkyldienyl”. More preferred cycloalkenyl radicals are “lower cycloalkenyl” radicals having four to about eight carbon atoms. Examples of such radicals include cyclobutenyl, cyclopentenyl and cyclohexenyl.

The terms “heterocyclyl”, “heterocycle” “heterocyclic” or “heterocyclo” refer to saturated, partially unsaturated and unsaturated heteroatom-containing ring-shaped radicals, which can also be called “heterocyclyl”, “heterocycloalkenyl” and “heteroaryl” correspondingly, where the heteroatoms may be selected from nitrogen, sulfur and oxygen. Examples of saturated heterocyclyl radicals include saturated 3 to 6-membered heteromonocyclic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. Heterocyclyl radicals may include a pentavalent nitrogen, such as in tetrazolium and pyridinium radicals. The term “heterocycle” also embraces radicals where heterocyclyl radicals are fused with aryl or cycloalkyl radicals. Examples of such fused bicyclic radicals include benzofuran, benzothiophene, and the like.

The terms “halogen” or “halo” as used herein, refers to an atom selected from fluorine, chlorine, bromine and iodine. Preferred halogens are fluorine and chlorine.

The term “haloalkyl” refers to an alkyl group which includes one or more halogen substituents.

The term “haloalkoxy” refers to an alkoxy group which includes one or more halogen substituents.

The term “substituted” refers to substitution by independent replacement of one, two, or three or more of the hydrogen atoms with substituents including, but not limited to, —F, —Cl, —Br, —I, —OH, C₁-C₁₂-alkyl; C₂-C₁₂-alkenyl, C₂-C₁₂-alkynyl, —C₃-C₁₂-cycloalkyl, protected hydroxy, —NO₂, —N₃, —CN, —NH₂, protected amino, oxo, thioxo, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O-C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-Cu-cycloalkyl, —CONH-aryl, —CONH-heteroaryl, —CONH-heterocycloalkyl, —OCO₂-C₁-C₁₂-alkyl, —OCO₂-C₂-C₈-alkenyl, —OCO₂-C₂-C₈-alkynyl, —OCO₂-C₃-C₁₂-cycloalkyl, —OCO₂-aryl, —OCO₂-heteroaryl, —OCO₂-heterocycloalkyl, -0O2-C₁-C₁₂ alkyl, —CO₂-C₂-C₈ alkenyl, —OCO₂-C₂-C₈ alkynyl, CO₂-C₃-C₁₂-cycloalkyl, —CO₂- aryl, CO₂-heteroaryl, CO₂-heterocyloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocyclo-alkyl, —NHC(O)H, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocyclo-alkyl, —NHCO₂-C₁-C₁₂-alkyl, —NHCO₂-C₂-C₈-alkenyl, —NHCO₂- C₂-C₈-alkynyl, —NHCO₂-C₃-C₁₂-cycloalkyl, —NHCO₂-aryl, —NHCO₂-heteroaryl, —NHCO₂- heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)-C₁-C₁₂-alkyl, —NHC(NH)-C₂-C₈-alkenyl, —NHC(NH)-C₂-C₈-alkynyl, —NHC(NH)-C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl, —SO₂NH₂, —SO₂NH—C₁-—SO₂NH—C₂-C₈-alkenyl, —SO₂NH—C₂-C₈-alkynyl, —SO₂NH—C₃-C₁₂-cycloalkyl, —SO₂NH-aryl, —SO₂NH-heteroaryl, —SO₂NH-heterocycloalkyl, —NHSO₂-C₁-C₁₂-alkyl, —NHSO₂-C₂-C₈-alkenyl, —NHSO₂-C₂-C₈-alkynyl, —NHSO₂-C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, -S-aryl, -S-heteroaryl, -S-heterocycloalkyl, or methylthio-methyl. In certain embodiments, the substituents are independently selected from halo, preferably Cl and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy; —CN; —OH; NH₂; C₁-C₄-alkylamino; di(C₁-C₄-alkyl)amino; and NO₂. It is understood that the aryls, heteroaryls, alkyls, cycloalkyls, heterocyclyls and the like can be further substituted. In some cases, each substituent in a substituted moiety is additionally optionally substituted when possible with one or more groups, each group being independently selected from C₁-C₄-alkyl; —CF₃, —OCH₃, —OCF₃, —F, —Cl, —Br, —I, —OH, —NO₂, —CN, and —NH₂. Preferably, a substituted alkyl group, such as a substituted methyl group, is substituted with one or more halogen atoms, more preferably one or more fluorine or chlorine atoms.

The term “optionally substituted”, as used herein, means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The compounds of the invention can occur in various forms, including salt forms, particularly pharmaceutically acceptable salts, co-crystals, solvates, hydrates, polymorphs, enantiomers, diastereoisomers, racemates and the like of the compounds having a formula as set forth herein. In certain embodiments, the compounds of the invention occur as a racemic mixture, for example of stereoisomers having the stereochemistry of Formulas (Ia), (1Ia), (IIIa), and (IVa) and Formulas (Ib), (IIb), (IIIb), and (IVb). In other embodiments, the compounds exist as mixtures of two enantiomers, with an enantiomeric excess of one enantiomer. In still other embodiments, the compounds exists as substantially pure single enantiomers, for example with an enatiomeric excess of one enantiomer of at least 90%, 95%, 98% or 99%. As used herein, the term “pharmaceutically acceptable salt,” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentane-propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include salts of an acid drug with nontoxic ammonium, quaternary ammonium, and amine cations.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in T. H. Greene and P. G. M. Wuts, Protective Groups in Organic Synthesis, 3rd edition, John Wiley & Sons, New York (1999). Examples of hydroxyl protecting groups include benzyloxycarbonyl, 4-methoxybenzyloxycarbonyl, tert-butoxy-carbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl (trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in T. H. Greene and P. G. M. Wuts, Protective Groups in Organic Synthesis, 3rd pnedition, John Wiley & Sons, New York (1999). Examples of amino protecting groups include, but are not limited to, methoxycarbonyl, t-butoxycarbonyl, 9-fluorenyl-methoxycarbonyl, benzyloxycarbonyl, and the like.

The term “protected amino,” as used herein, refers to an amino group protected with an amino protecting group as defined above.

The present invention includes all pharmaceutically acceptable isotopically-labeled or enriched compounds of the invention. These compounds include at one or more positions an isotopic abundance or the indicated element which differs from the natural isotopic distribution for that element. For example, a position at which a hydrogen atom is depicted can include deuterium at a higher abundance than the natural abundance of deuterium.

Examples of isotopes suitable for inclusion in the compounds of the invention comprises isotopes of hydrogen, such as ²H and ³H, carbon, such as ¹¹C ¹³C and ¹⁴C, nitrogen, such as ¹³N and ¹⁵N, oxygen, such as ¹⁵O, ¹⁷O and ¹⁸O, chlorine, such as ³⁶Cl, fluorine, such as¹⁸F, iodine, ¹²³I and ¹²⁵I, phosphorus, such as ³²P, and sulfur, such as ³⁵S.

Substituents indicated as attached through variable points of attachments can be attached to any available position on the ring structure.

As used herein, the term “therapeutically effective amount of the subject compounds,” with respect to the subject method of treatment, refers to an amount of the subject compound which, when delivered as part of desired dose regimen, brings about management of the disease or disorder to clinically acceptable standards.

“Treatment” or “treating” refers to an approach for obtaining beneficial or desired clinical results in a patient. For purposes of this invention, beneficial or desired clinical results include, but are not limited to, one or more of the following: alleviation of symptoms, diminishment of extent of a disease, stabilization (i.e., not worsening) of a state of disease, preventing spread (i.e., metastasis) of disease, preventing occurrence or recurrence of disease, delay or slowing of disease progression, amelioration of the disease state, and remission (whether partial or total).

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### EXAMPLES

List of Abbreviations:

All temperatures are in degrees Centigrade

BF_{3.}Et₂O—boron trifluoride etherate

CDCl₃—deuterated chloroform

CF—cystic fibrosis

CFTR—cystic fibrosis transmembrane conductance regulator

CH₃CN—acetonitrile

CH₃NO₂—nitromethane

CH₂Cl₂—methylene chloride

DIPEA—N,N-diisopropylethylamine

DMF—dimethylformamide

DMSO-d₆—deuterated dimethylsulfoxide

ENaC—epithelial sodium channel

Et₂O—diethyl ether

EtOAc—ethyl acetate

H20—water

HATU—(1-[Bis(dimethylamino)methylene]-1H-1,2,3-triazolo[4,5-b]pyridinium 3-oxid hexafluorophosphate)

FIBS—Hepes-buffered saline

HCl—hydrochloric acid

HCOOH—formic acid

HPLC—high pressure liquid chromatography

hr—hours

HTS—high throughput screen

ms—milliseconds

Na₂SO₄—sodium sulfate

NaBH₄—sodium borohydride

NaOH—sodium hydroxide

NaHCO₃—sodium bicarbonate

NAUC—normalized area under the curve

NH4OAc—ammonium acetate

NMR—nuclear magnetic resonance

Pet. Ether—petroleum ether

PBS—Phosphate buffered saline

Pd(PPh₃)₄—palladium tetrakis

s—seconds

rt—RT

TFA—trifluoroacetic acid

THF—tetrahydrofuran

YFP—yellow fluorescent protein

### Example 1

### Synthesis of Compounds

Compounds of the invention were synthesized via either method A or method B below. Method A proceeds via ring opening of the corresponding succinimide compounds. These succinimide compounds were prepared as described in WO 2017/117239, which is incorporated herein by reference in its entirety.

Method A

This method is exemplified for the preparation of 5,7-dichloro-1′-((3,5-dichloro-4-(difluoromethoxy)phenyl)carbamoyl)-6′,6′-dimethyl-2-oxo-1′,2′,5′,6′,7′,7a′-hexahydrospiro[indoline-3,3′-pyrrolizine]-2′-carboxylic acid (Compound 8) as illustrated in the scheme below.

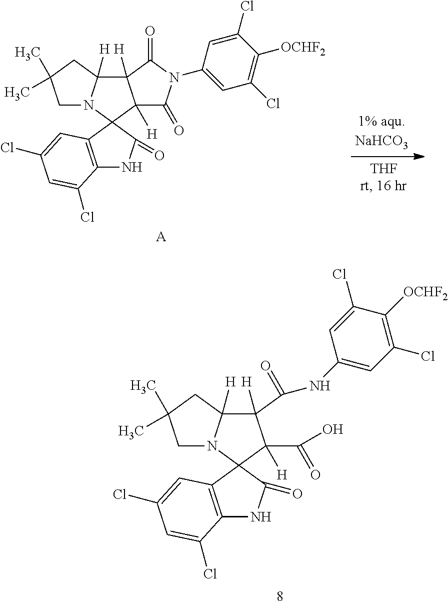

To a stirred solution of Compound A (100 mg, 0.165 mmol) in THF (33 mL) was added 1% aqu. NaHCO₃ (66 mL) at rt, and the reaction mixture was stirred at rt for 24 hr. The reaction mixture was cooled to 0° and acidified with 1N HCl (pH ˜4), then extracted with EtOAc (2×30 mL). The combined organic layers were dried over the anhydrous Na₂SO₄, filtered and evaporated under vacuum to afford crude product. This was purified by preparative HPLC (0.05% HCOOH/CH₃CN/H₂O) to give 40 mg (39% yield) of 8 as solid. LCMS: 621.9 [M+H]⁺; (97.2% purity). ¹H NMR (500 MHz, DMSO-d₆) δ=12.28 (bs, 1H), 10.89 (s, 1H), 10.51 (s, 1H), 8.16 (d, J=2.0 Hz, 1H), 7.82 (s, 2H), 7.45 (d, J=2.0 Hz, 1H), 7.11 (t, J=72.5 Hz, 1H), 4.20-4.13 (m, 2H), 3.47 (t, J=7.0 Hz, 1H), 2.49 (d, J=8.0 Hz, 1H), 1.93 (d, J=7.5 Hz, 1H), 1.61-1.58 (m, 1H), 1.29-1.23 (m, 1H), 1.00 (s, 3H), 0.91 (s, 3H); ¹⁹F NMR (470.59 MHz, DMSO-d₆): −80.26, −80.10. Chiral SFC purification (97.9% ee).

Prep HPLC Conditions

Column: SYMMETRY-C8 (300*19), 7u; Mobile phase: 0.1% FORMIC ACID IN H₂O: CH₃CN GRADIENT: (T%B): 0/30,8/80,8.1/98,10/98,10.1/30,13/30 Flow Rate: 20 ml/min; Diluent: CH₃CN +H₂O+THF.

Method B

This method is exemplified for the preparation of (1′R,2′S,3R,7a′R)-1′-((3-(tert-butyl)phenethyl)carbamoyl)-5,7-dichloro-6′,6′-difluoro-2-oxo-1′,2′,5′,6′,7′,7a′-hexahydrospiro[indoline-3,3′-pyrrolizine]-2′-carboxylic acid (Compound 44)

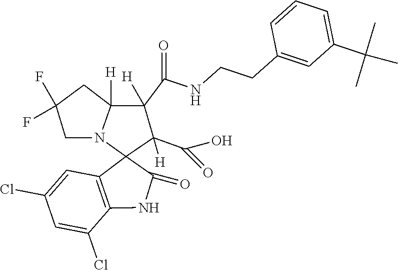 [SMILES: CC(C)(C)c1cccc(CCNC(=O)C2C3CC(F)(F)CN3C3(C(=O)Nc4c(Cl)cc(Cl)cc43)C2C(=O)O)c1]

### (Z)-4-(allyloxy)-4-oxobut-2-enoic acid (A)

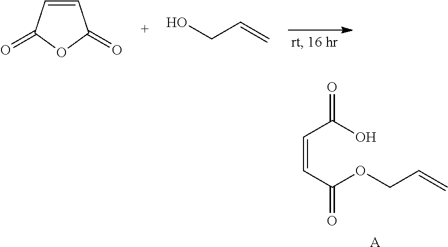

A solution of maleic anhydride (15.0 g, 152 mmol) in allyl alcohol (200 mL) was stirred at rt for 16 hr. The reaction mixture was concentrated in vacuo to a residue which was dissolved in Et₂O (1.0 L), washed with H₂O (3×1 L), brine (500 mL) then dried over anhydrous Na₂SO₄ and concentrated in vacuo to give 12.0 g (50% yield) of A as a colorless liquid. LCMS: m/z 157.0 [M+H]⁺; (99.5% purity).

### 2′-((allyloxy)carbonyl)-5,7-dichloro-6′,6′-difluoro-2-oxo-1′,2′,5′,6′,7′,7a′-hexahydrospiro[indoline-3,3′-pyrrolizine]-1′-carboxylic acid (E)

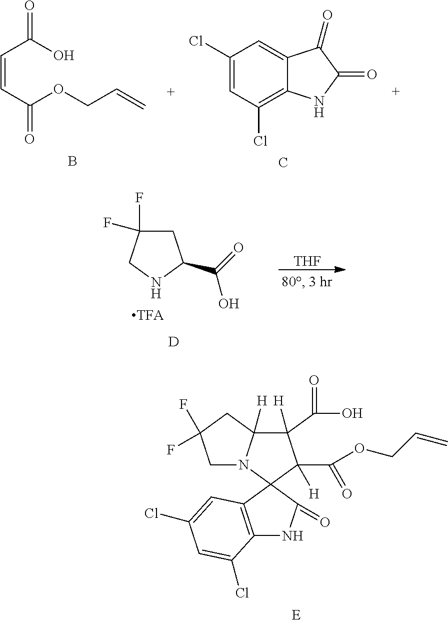

To a stirred solution of B (5 g, 32.0 mmol) in THF (70 mL) was added 5,7-dichloroisatin (C) (6.9 g, 32.0 mmol) and 4,4-difluoro-L-proline.TFA salt (D) (7.9 g, 32.0 mmol) at rt. The reaction mixture was stirred at 80° for 3 hr, then cooled to rt, and evaporated in vacuo to give the crude product as a mixture of diastereomers. This was purified by column chromatography, eluting with Pet. Ether:EtOAc (80° to give crude product as a brown solid. This brown material was triturated with CH₂Cl₂ and filtered to give, after drying in vacuo, 1.3 g (9% yield) of E as a white solid (single isomer). LCMS: m/z 461.0 [M+H]⁺; (99.2% purity). ¹H NMR (500 MHz, acetone-d6) δ 11.8-10.9 (br s, 1H), 9.96 (s, 1H), 7.85 (d, J=2 Hz, 1H), 7.39 (d, J=2 Hz, 1H), 5.56-5.53 (m, 1H), 5.15-5.07 (m, 2H), 4.33-4.32 (m, 2H), 4.22-4.20 (m, 1H), 4.10-4.09 (m, 1H), 3.82-3.79 (m, 1H), 3.30-3.25 (m, 1H), 2.53-2.45 (m, 1H), 2.34-2.20 (m, 1H). The desired diastereomer (i.e., having the relative stereochemistry set forth in Formulas (Ia), (IIa) and (IIIa)) was confirmed by 2D NMR.

### (E)-1-(tert-butyl)-3-(2-nitrovinyl)benzene (F)

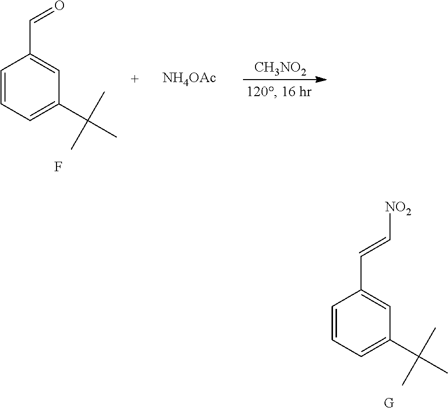

To a solution of NH₄OAc (4.2 g, 55.1 mmol) in CH₃NO₂ (150 mL) was added 3-(tert-butyl)benzaldehyde (F) (3 g, 18.3 mmol) at 90° and the resulting mixture was stirred at 120° for 16 hr. The reaction mixture was cooled to rt and concentrated in vacuo to give the crude product as a brown liquid. This was purified by column chromatography, eluting with Pet. Ether:EtOAc (80:20) to give 2.9 g (65% yield) of product G as a pale brown solid. ¹H NMR (500 MHz, CDCl₃) δ 8.03 (d, J=13.5 Hz, 1H), 7.60 (d, J=14 Hz, 1H), 7.55-7.53 (m, 2H), 7.39-7.38 (m, 2H).

### 2-(3-(tert-butyl)phenyl)ethan-1-amine (I)

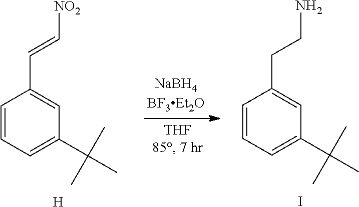

To a stirred solution of NaBH₄ (847 mg, 22.4 mmol) in anhydrous THF (20 mL) was added BF3.Et₂O (3.3 mL, 26.8 mmol) at 0°. After stirring at 0° for 15 minutes, the reaction mixture was warmed to rt and then a solution of (H) (920 mg, 4.48 mmol) in anhydrous THF (10 mL) was added. The resulting mixture was stirred at 85° for 5 hr, then cooled to 0°. H₂O (40 mL) and 1N HCl (40 mL) were added to the reaction mixture over 20 minutes, and the resulting mixture was stirred at 85° for 2 hr. The reaction mixture was cooled to 0° and basified (pH ˜12) using 5N NaOH solution. The resulting mixture was extracted with EtOAc (2×100 mL), then the combined organic layers were washed with brine, dried over anhydrous Na₂SO₄, and concentrated in vacuo to give 700 mg (88% yield) of product (I) as liquid which was used in the next step without purification. LCMS: m/z 178.1 [M+H]⁺; (99.5% purity).

### Allyl 1′-((3-(tert-butyl)phenethyl)carbamoyl)-5,7-dichloro-6′,6′-difluoro-2-oxo-1′,2′,5′,6′,7′,7a′-hexahydrospiro[indoline-3,3′-pyrrolizine]-2′-carboxylate (J)

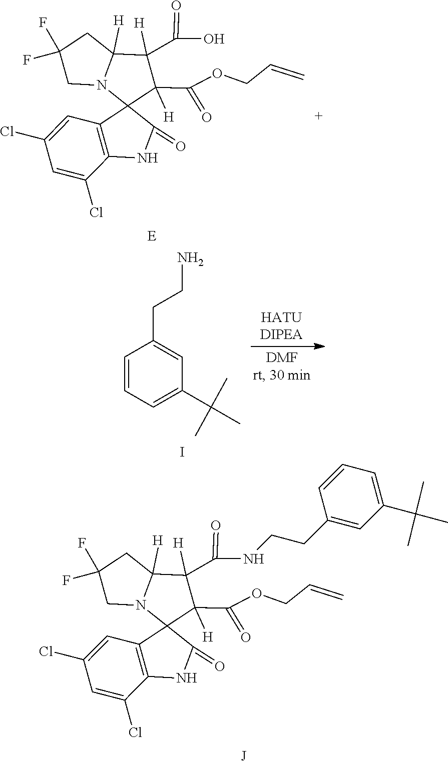

To a stirred solution of E (200 mg, 0.43 mmol) in DMF (10 mL) was added DIPEA (0.15 mL, 0.86 mmol), HATU (196 mg, 0.51 mmol) and I (115 mg, 0.65 mmol) at rt. The resulting reaction mixture was stirred at rt for 30 min, then poured into ice cold H₂O and stirred for 10 minutes. The resulting precipitate was collected, washed with cold H₂O and dried in vacuo to give 250 mg (94% yield) of product J as a white solid, which was used in the next step without purification. LCMS: m/z 620.1 [M+H]⁺; (10.9%+83.3% purity).

### 1′-((3-(tert-butyl)phenethyl)carbamoyl)-5,7-dichloro-6′,6′-difluoro-2-oxo-1′,2′,5′,6′,7′,7a′-hexahydrospiro[indoline-3,3′-pyrrolizine]-2′-carboxylic acid (44).

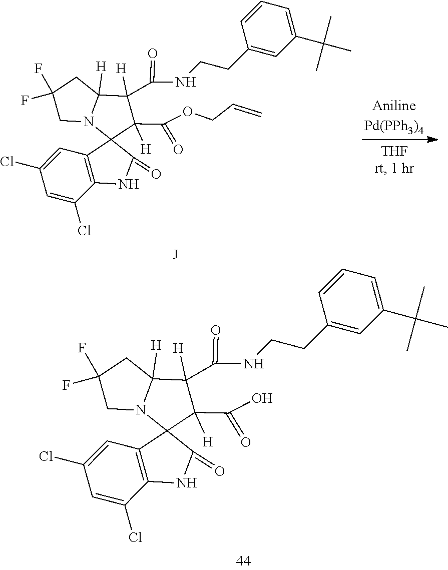 [SMILES: C=CCOC(=O)C1C(C(=O)NCCc2cccc(C(C)(C)C)c2)C2CC(F)(F)CN2C12C(=O)Nc1c(Cl)cc(Cl)cc12 ; CC(C)(C)c1cccc(CCNC(=O)C2C3CC(F)(F)CN3C3(C(=O)Nc4c(Cl)cc(Cl)cc43)C2C(=O)O)c1]

To a stirred solution of J (200 mg, 0.32 mmol) in anhydrous THF (5 mL) were added aniline (30 mg, 0.32 mmol) and Pd(PPh₃)₄ (37 mg, 0.032 mmol) at rt. The resulting mixture was stirred at rt for 1 hr, then diluted with EtOAc (100 mL) and washed with brine, dried over anhydrous Na₂SO₄, and concentrated in vacuo to give the crude product as liquid. The crude product was purified by preparative HPLC using the following conditions to give 95 mg (50% yield) of product 44: Column—INERTSIL-ODS (250 mm×20 mm×5 uM); Mobile Phase-A—0.1% Formic Acid in H₂O, B—0.05% Formic Acid in CH₃CN; Time (min)/%B: 0/60, 8/85, 9/85, 12/95, 12.1/60, 15/60; Flow Rate—20 mL/min.

LCMS: m/z 580.1 [M+H]⁺; (98.9% de). ¹H NMR (500 MHz, DMSO-d₆): δ 12.40 (s, 1H), 10.95 (s, 1H), 8.25 (br s, 2H), 7.43 (d, J=1.5 Hz, 1H), 7.26 (s, 1H), 7.24-7.20 (m, 2H), 7.07-7.05 (m, 1H), 3.96-3.90 (m, 2H), 3.46-3.44 (m, 2H), 3.17- 3.13 (m, 2H), 2.77-2.71 (m, 2H), 2.58-2.51 (m, 1H), 2.10-2.07 (m, 1H), 1.99-1.85 (m, 1H), 1.28 (s, 9H).

Compounds 1-65 were prepared as described herein and characterized by LCMS. For each compound, the synthesis method used is shown in the final column.

| | | Single enantiomer | |
|---|---|---|---|
| Compound | | (s) or mixture of | Synthesis |
| No. | M/Z | stereoisomers (m) | method |
| 1. | 623.2 [M + H]⁺ | (s) | A |
| 2. | 556.0 [M + H]⁺ | (s) | A |
| 3. | 558.0 [M − H]⁻ | (s) | A |
| 4. | 623.9 [M − H]⁻ | (s) | A |
| 5. | 627.0 [M − H]⁻ | (s) | A |
| 6. | 589.0 [M − H]⁻ | (s) | A |
| 7. | 561.9 [M − H]⁻ | (s) | A |
| 8. | 622.0 [M − H]⁻ | (s) | A |
| 9. | 593.9 [M − H]⁻ | (s) | A |
| 10. | 529.0 [M − H]⁻ | (s) | A |
| 11. | 564.9 [M − H]⁻ | (s) | A |
| 12. | 620.0 [M + H]⁺ | (s) | A |
| 13. | 553.9 [M − H]⁻ | (s) | A |
| 14. | 585.9 [M − H]⁻ | (s) | A |
| 15. | 528.0 [M − H]⁻ | (s) | A |
| 16. | 554.0 [M − H]⁻ | (s) | A |
| 17. | 612.2 [M − H]⁻ | (s) | A |
| 18. | 596.4 [M − H]⁻ | (s) | A |
| 19. | 593.9 [M − H]⁻ | (s) | A |
| 20. | 627.9 [M − H]⁻ | (s) | A |
| 21. | 556.2 [M + H]⁺ | (s) | A |
| 22. | 642.9 [M − H]⁻ | (s) | A |
| 23. | 566.0 [M + H]⁺ | (s) | A |
| 24. | 617.9 [M − H]⁻ | (m) | A |
| 25. | 653.9 [M − H]⁻ | (m) | A |
| 26. | 584.0 [M − H]⁻ | (m) | A |
| 27. | 487.0 [M − H]⁻ | (m) | A |
| 28. | 516.1 [M − H]⁻ | (s) | A |
| 29. | 530.4 [M + H]⁺ | (s) | A |
| 30. | 605.0 [M − H]⁻ | (m) | A |
| 31. | 619.0 [M − H]⁻ | (m) | A |
| 32. | 607.9 [M − H]⁻ | (m) | A |
| 33. | 530.0 [M − H]⁻ | (m) | A |
| 34. | 578.0 [M − H]⁻ | (m) | A |
| 35. | 555.9 [M − H]⁻ | (m) | A |
| 36. | 649.9 [M − H]⁻ | (m) | A |
| 37. | 545.9 [M − H]⁻ | (m) | A |
| 38. | 570.0 [M − H]⁻ | (m) | A |
| 39. | 614.0 [M − H]⁻ | (m) | A |
| 40. | 555.9 [M − H]⁻ | (s) | A |
| 41. | 567.9 [M − H]⁻ | (m) | A |
| 42. | 581.9 [M + H]⁺ | (m) | A |
| 43. | 567.9 [M − H]⁻ | (s) | A |
| 44. | 580.1 [M + H]⁺ | (m) | B |
| 45. | 572.1 [M + H]⁺ | (m) | B |
| 46. | 580.1 [M + H]⁺ | (m) | B |
| 47. | 538.0 [M + H]⁺ | (m) | B |
| 48. | 538.0 [M + H]⁺ | (m) | B |
| 49. | 538.0 [M + H]⁺ | (m) | B |
| 50. | 558.0 [M + H]⁺ | (m) | B |
| 51. | 558.0 [M + H]⁺ | (m) | B |
| 52. | 558.0 [M + H]⁺ | (m) | B |
| 53. | 544.2 [M + H]⁺ | (m) | B |
| 54. | 566.1 [M + H]⁺ | (m) | B |
| 55. | 681.0 [M + H]⁺ | (s) | A |
| 56. | 611.9 [M + H]⁺ | (s) | A |
| 57. | 578.1 [M + H]⁺ | (m) | B |
| 58. | 609.9 [M − H]⁻ | (s) | A |
| 59. | 564.1 [M + H]⁺ | (s) | A |
| 60. | 564.1 [M + H]⁺ | (s) | A |
| 61. | 628.2 [M + H]⁺ | (m) | A |
| 62. | 627.9 [M + H]⁺ | (m) | A |
| 63. | 648.1 [M + H]⁺ | (m) | A |
| 64. | 608.3 [M + H]⁺ | (m) | B |
| 65. | 646.1 [M − H]⁻ | (m) | A |

### Example 66

### Synthesis of 5,7-dichloro-4′-((3,5-dichlorophenyl)carbamoyl)-5′-(difluoro(pyridin-4-yl)methyl)-2-oxospiro[indoline-3,2′-pyrrolidine]-3′-carboxylic acid, 66.9a, 66.9b1, 66.9b2, 66.9b3

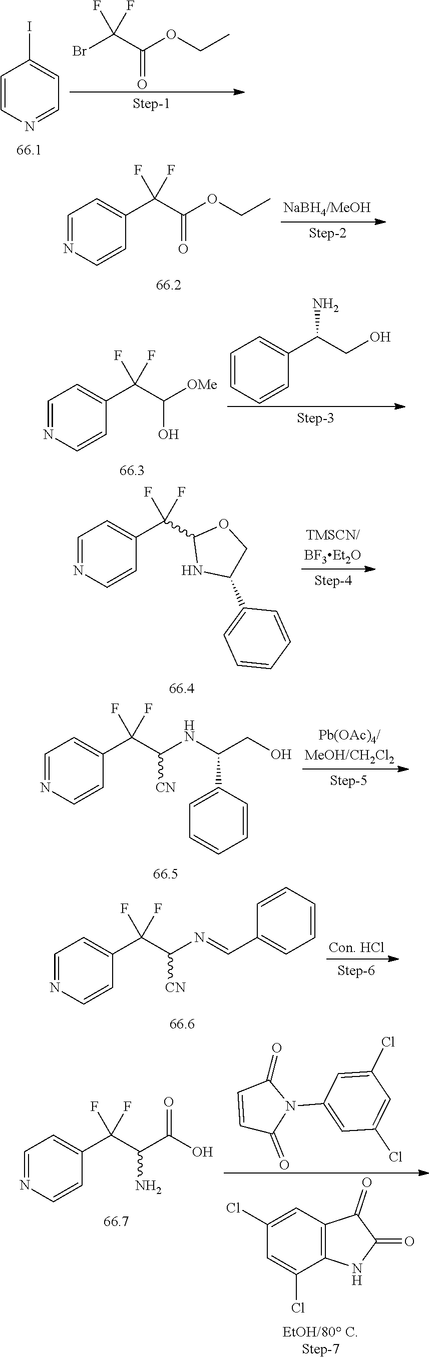

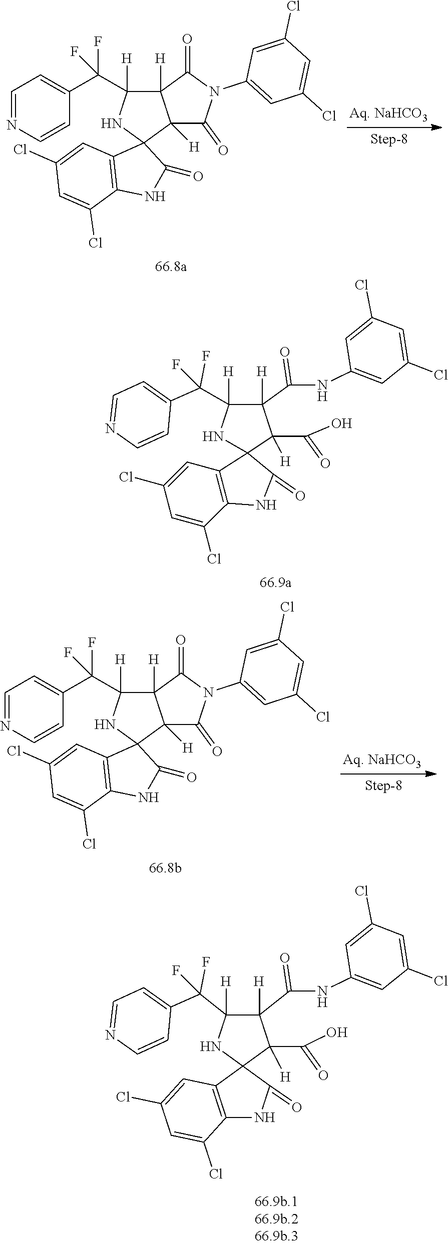 [SMILES: O=C1C2C(C(F)(F)c3ccncc3)NC3(C(=O)Nc4c(Cl)cc(Cl)cc43)C2C(=O)N1c1cc(Cl)cc(Cl)c1 ; O=C(Nc1cc(Cl)cc(Cl)c1)C1C(C(F)(F)c2ccncc2)NC2(C(=O)Nc3c(Cl)cc(Cl)cc32)C1C(=O)O ; O=C1C2C(C(F)(F)c3ccncc3)NC3(C(=O)Nc4c(Cl)cc(Cl)cc43)C2C(=O)N1c1cc(Cl)cc(Cl)c1 ; O=C(Nc1cc(Cl)cc(Cl)c1)C1C(C(F)(F)c2ccncc2)NC2(C(=O)Nc3c(Cl)cc(Cl)cc32)C1C(=O)O]

### Synthesis of 66.2

To a stirred suspension of activated Cu powder (3.8 g, 60.9 mmol), CuI (2.3 g, 12.2 mmol) in DMSO (30 mL) was added 4-iodopyridine, 66.1 (5.0 g, 24.4 mmol) and ethyl 2-bromo-2,2-difluoroacetate (12.4 g, 60.9 mmol) at RT. The resulting reaction mixture was stirred for 16 h at RT. The reaction mixture was cooled to RT, poured into ice-water (50 mL) and extracted with diethyl ether (2×50 mL). The combined organic layer was dried over anhydrous Na₂SO₄, filtered and concentrated under reduced pressure. The residue was purified by flash column chromatography (80 g Silica gel cartridge, 20% EtOAc in pet ether) to afford 66.2 (4 g, 81%) as liquid. ¹H NMR (500 MHz, CDCl₃): 8.81 (br s, 2H), 7.53 (d, J=5.0 Hz, 2H), 4.32 (q, J=7.0 Hz, 2H), 1.32 (t, J=7.0 Hz, 3H).

### Synthesis of 66.3

To a stirred solution of 66.2 (2.2 g, 10.9 mmol) in MeOH (50 mL) was added NaBH₄ (289 mg, 7.6 mmol) portion wise at −60° C. The resulting reaction mixture was stirred for 4 h at −60° C. The reaction mixture was quenched with NH₄Cl solution at −60° C. and extracted with EtOAc (2×30 mL). The combined organic layer was washed with water (30 mL), brine (30 mL), dried over anhydrous Na₂SO₄, filtered and concentrated under reduced pressure to afford 66.3 (1.8 g) as a solid, which used in the next step without purification. ¹H NMR (400 MHz, DMSO-d₆): 8.90 (br s, 2H), 7.56 (br s, 2H), 7.33 (d, J=6.4 Hz, 1H), 4.89 (q, J=6.0 Hz, 1H), 3.29 (s, 3H).

### Synthesis of 66.4

To a stirred solution of 66.3 (1.8 g, 9.49 mmol) in toluene (30 mL) was added (S)-2-amino-2-phenylethan-1-ol (1.3 g, 9.49 mmol), PTSA (180 mg, 0.94 mmol) at RT. The resulting mixture was refluxed for 2 h using Dean-Stark apparatus. The reaction mixture was concentrated under reduced pressure. The resulting residue was purified by flash column chromatography (40 g Silica gel cartridge, 10% EtOAc in pet ether) to afford mixture of diastereomers 66.4 (2.1 g, 80%) as a colorless liquid. LCMS: (37.2+52.1%, m/z [M+H]⁺=277.0.

### Synthesis of 66.5

To a stirred solution of 66.4 (2.0 g, 7.23 mmol) in CH₂Cl₂ (50 mL) was added TMSCN (1.4 g, 14.5 mmol) and BF_{3.}Et₂O (2 g, 14.5 mmol) at −78° C. over a period of 10 minutes. The resulting reaction mixture was stirred at −78° C. for 8 h. The reaction mixture was poured into sat.NaHCO₃ solution (80 mL) and extracted with CH₂Cl₂ (2×30 mL). The combined organic layer was washed with water (50 mL), brine (50 mL), dried over anhydrous Na₂SO₄, filtered and concentrated under vacuum to afford residue. The residue was purified by flash column chromatography (40 g Silica gel cartridge, 30% EtOAc in pet ether) to afford mixture of diastereomers 66.5 (1.8 g, 81%) as a pale brown solid. LCMS: (30.5+66.2%), m/z [M+H]⁺=304.3.

### Synthesis of 66.6

To a stirred solution of 66.5 (2.4 g, 7.91 mmol) in MeOH (50 mL) and CH₂Cl₂ (80 mL) was added Pb(OAc)₄ (5.2 g, 11.9 mmol) at 0° C. The resulting reaction mixture was stirred for 2 h at 0° C. The reaction mixture was poured in to 0.2M phosphate buffer solution (50 mL) at RT and then filtered through celite bed. The aqueous layer was extracted with CH₂Cl₂ (2×20 mL). The combined organic layer was washed with H₂O (2×50 mL), brine (50 mL), dried over anhydrous Na₂SO₄, filtered and concentrated under vacuum to afford mixture of diastereomers 66.6 (1.8 g) as a brown sticky material, which used in the next step without purification.

### Synthesis of 66.7

To a stirred solution of 66.6 (1.7 g, 6.26 mmol) in con. HCl (60 mL) was heated at 100° C. for 6 h. The reaction mixture was cooled to RT and concentrated under vacuum at 50° C. The resulting residue was triturated with CH₂Cl₂ to afford a mixture of diastereomers 66.7 (550 mg) as a pale brown solid. LCMS: 87.5%, m/z [M+H]⁺=203.2.

### Synthesis of 66.8a & 66.8b

To a stirred solution of 66.7 (500 mg, 2.47 mmol) in EtOH (40 mL) was added 1-(3,5-dichlorophenyl)-1H-pyrrole-2,5-dione (598 mg, 2.47 mmol), 5,7-dichloroindoline-2,3-dione (534 mg, 2.47 mmol) at RT. The reaction mixture was stirred at 90° C. for 2 h. The volatile components were removed under vacuum. The resulting residue was purified by flash chromatography (Silica gel 100-200 mesh, 50% EtOAc/pet ether) to afford 66.8a (120 mg, 8%) and 66.8b (240 mg, 16%) as a solid.

66.8a: ¹H NMR (500 MHz, DMSO-d₆): 11.18 (s, 1H), 8.76 (d, J=5.0 Hz, 2H), 7.78 (d, J=1.5 Hz, 1H), 7.58 (d, J=6.0 Hz, 2H), 7.53 (d, J=2.0 Hz, 1H), 7.33 (d, J=2.0 Hz, 1H), 7.25 (d, J=2.0 Hz, 2H), 4.60-4.49 (m, 2H), 3.93 (s, 2H); LCMS: 97.5%, m/z [M+H]⁺=597.1. 66.8b: ¹H NMR (500 MHz, DMSO-d₆): 11.07 (br s, 1H), 8.73 (d, J=5.5 Hz, 2H), 7.78 (t, J=2.0 Hz, 1H), 7.61 (d, J=6.0 Hz, 2H), 7.49 (d, J=1.5 Hz, 1H), 7.33 (d, J=1.5 Hz, 2H), 7.05 (d, J=1.5 Hz, 1H), 5.02-4.94 (m, 1H), 4.35 (d, J=5.0 Hz, 1H), 3.90 (t, J=8.5 Hz, 1H), 3.67 (d, J=8.0 Hz, 1H); LCMS: 99.4%, m/z [M+H]⁺=597.1.

### Synthesis of 66.9a

To a stirred solution of 66.8a (80 mg, 0.13 mmol) in THF (30 mL) was added 1% NaHCO₃ solution (60 mL) at RT. The resulting reaction mixture was stirred for 96 h at RT. After completion of the reaction, the pH of the solution was adjusted to ˜6-7 with 1N HCl solution and extracted with EtOAc (2×20 mL). The combined organic layer was washed with brine (30 mL), dried over anhydrous Na₂SO₄, filtered and concentrated under reduced pressure. The resulting residue was purified by prep. HPLC [X-BRIDGE C18 (150×30) mm, 5 μ; A: 0.1% HCOOH in H₂O, B: Acetonitrile; Gradient: (Time/%B): 0/40, 8/80, 9/80, 9.1/98, 14/98, 14.1/40, 17/40 at 25 mL/min] to give 66.9a (32 mg, 39%) as a solid. ¹H NMR (500 MHz, DMSO-d₆): 12.91 (br s, 1H), 11.21 (br s, 1H), 11.03 (br s, 1H), 8.71 (d, J=5.5 Hz, 2H), 7.54-7.52 (m, 4H), 7.44 (br s, 1H), 7.28 (s, 1H), 7.25-7.16 (m, 1H), 4.80-4.69 (m, 1H), 4.30-4.15 (m, 2H), 3.54-3.51 (m, 1H); LCMS: 97.0%, m/z [M+H]⁺=615.1.

### Synthesis of 66.9b.1, 66.9b.2 & 66.9b.3

To a stirred solution of 66.8b (180 mg, 0.3 mmol) in THF (60 mL) was added 1% NaHCO₃ solution (120 mL) at RT. The resulting reaction mixture was stirred for 96 h at RT. After completion of the reaction, the pH of the solution was adjusted to ˜6-7 with 1N HCl solution and extracted with EtOAc (2×20 mL). The combined organic layer was washed with brine (60 mL), dried over anhydrous Na₂SO₄, filtered and concentrated under reduced pressure. The resulting residue was purified by prep. HPLC [X-BRIDGE C₁₈ (150×30) mm, 5 μ; A: 0.1% HCOOH in H₂O, B: Acetonitrile; Gradient: (Time/%B): 0/45, 8/80, 10/80, 10.1/98, 12/98, 12.1/45, 15/45 at 25 mL/min] to afford 66.9b.1 (7 mg, 4%) as a solid, 66.9b.2 (7 mg, 4%) as a solid and 66.9b.3 (64 mg, 35%) as a solid.

66.9b.1: LCMS: 93.8%, m/z [M+H]⁺=615.1;

66.9b.2: LCMS: 87.0%, m/z [M+H]⁺=615.1;

66.9b.3: ¹H NMR (500 MHz, DMSO-d₆): 12.46 (br s, 1H), 10.95 (s, 1H), 10.57 (s, 1H), 8.66 (d, J=5.0 Hz, 2H), 8.19 (d, J=1.5 Hz, 1H), 7.67 (d, J=1.5 Hz, 2H), 7.51 (d, J=6.0 Hz, 2H), 7.46 (s, 1H), 7.32 (s, 1H), 4.34-4.28 (m, 1H), 4.00-3.90 (m, 1H), 3.76-3.73 (m, 1H), 3.60-3.59 (m, 1H); LCMS: 99.4%, m/z [M+H]⁺=615.1.

### Example 67

### Synthesis of 5,7-dichloro-5,7′-((4-chlorophenyl)difluoromethyl)-4′-((3,5-dichlorophenyl)carbamoyl)-2-oxospiro[indoline-3,2′-pyrrolidine]-3′-carboxylic acid, 67.9a & 67.9b

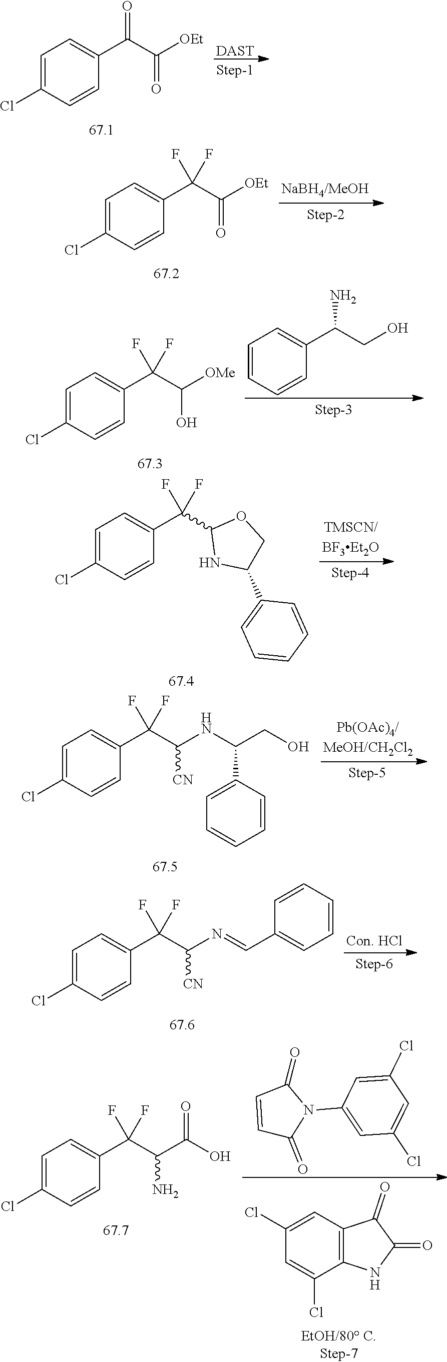

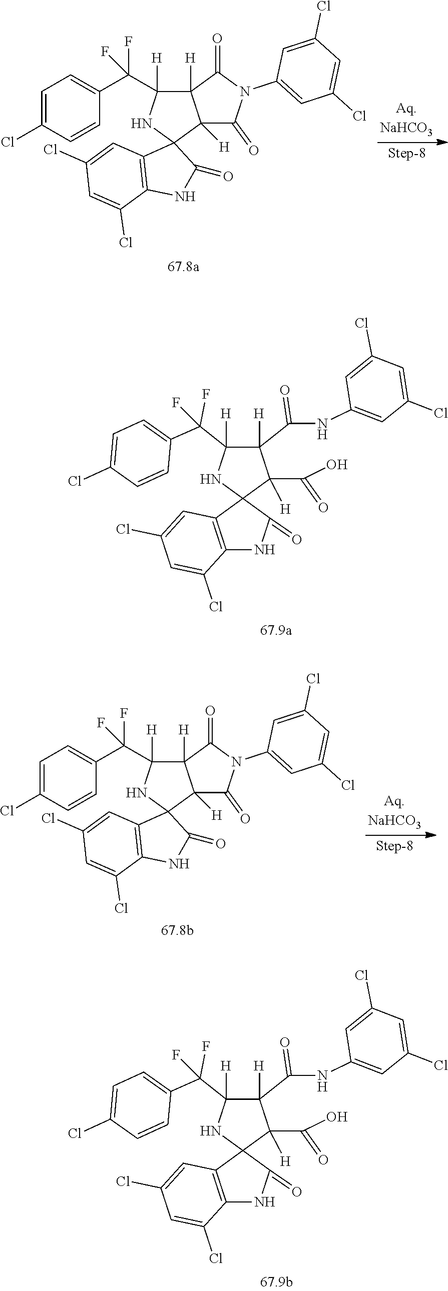 [SMILES: O=C1C2C(C(F)(F)c3ccc(Cl)cc3)NC3(C(=O)Nc4c(Cl)cc(Cl)cc43)C2C(=O)N1c1cc(Cl)cc(Cl)c1 ; O=C(Nc1cc(Cl)cc(Cl)c1)C1C(C(F)(F)c2ccc(Cl)cc2)NC2(C(=O)Nc3c(Cl)cc(Cl)cc32)C1C(=O)O ; O=C1C2C(C(F)(F)c3ccc(Cl)cc3)NC3(C(=O)Nc4c(Cl)cc(Cl)cc43)C2C(=O)N1c1cc(Cl)cc(Cl)c1 ; O=C(Nc1cc(Cl)cc(Cl)c1)C1C(C(F)(F)c2ccc(Cl)cc2)NC2(C(=O)Nc3c(Cl)cc(Cl)cc32)C1C(=O)O]

### Synthesis of 67.2

To a stirred solution of ethyl 2-(4-chlorophenyl)-2-oxoacetate, 67.1 (5.0 g, 23.6 mmol) in DCM (50 mL) was added DAST (11 mL, 82.5 mmol) at RT. The resulting reaction mixture was stirred for 16 h at RT. The reaction mixture was cooled to 0° C. and quenched with sat. NaHCO₃ solution. The organic layer was separated, washed with water (2×50 mL) and brine (15 mL). The combined organic layer was dried over anhydrous Na₂SO₄, filtered and concentrated under reduced pressure to afford 67.2 (5.0 g) as a pale brown liquid, which was used in the next step without purification.

¹H NMR (400 MHz, CDCl₃): 7.55 (d, J=8.8 Hz, 2H), 7.44 (d, J=8.8 Hz, 2H), 4.30 (q, J=7.2 Hz, 2H), 1.31 (t, J=7.2 Hz, 3H).

### Synthesis of 67.3

To a stirred solution of 67.2 (5 g, 21.3 mmol) in MeOH (25 mL) was added NaBH₄ (800 mg, 21.3 mmol) portion wise at −60° C. The resulting reaction mixture was stirred for 1 h at −60° C. The reaction mixture was quenched with 1N HCl solution (50 mL) at 0° C. and extracted with Et₂O (2×30 mL). The combined organic layer was washed with water (30 mL), brine (30 mL), dried over anhydrous Na₂SO₄, filtered and concentrated under reduced pressure to afford 67.3 (5.0 g) as pale yellow liquid, which used for next step without purification and analysis.

### Synthesis of 67.4

To a stirred solution of 67.3 (5.0 g, 22.4 mmol) in toluene (50 mL) was added (S)-2-amino-2-phenylethan-1-ol (2.99 g, 22.4 mmol), PTSA (112 mg, 0.44 mniol) at RT. The reaction mixture was refluxed for 1 h using Dean-Stark apparatus. Then the reaction mixture was concentrated under reduced pressure. The residue was purified by flash column chromatography (40 g Silica gel cartridge, 20% EtOAc in pet ether] to afford mixture of diastereomers 67.4 (6.5 g, 94%) as a colorless liquid. LCMS: (47.7+40.0)%, m/z [M+H]⁺=310.

### Synthesis of 67.5

To a stirred solution of 67.4 (6.5 g, 21.0 mmol) in CH₂Cl₂ (100 mL) was added TMSCN (5.2 mL, 42.0 mmol) and BF3.Et₂O (5.1 mL, 42.0 mmol) at −78° C. over a period of 10 minutes. The resulting reaction mixture was stirred at RT for 16 h. The reaction mixture was cooled to 0° C., quenched with sat.NaHCO₃ solution (80 mL) and extracted with CH₂Cl₂ (2×30 mL). The combined organic layer was washed with water (50 mL), brine (50 mL), dried over anhydrous Na₂SO₄, filtered and concentrated under vacuum. The residue was purified by flash column chromatography (80 g Silica gel cartridge, 20% EtOAc in pet ether) to afford mixture of diastereomers 67.5 (5.8 g, 82%) as a solid. ¹H NMR (400 MHz, CDCl₃): 7.52-7.22 (m, 7H), 7.02 (d, J=7.6 Hz, 2H), 4.13-3.96 (m, 1H), 3.81-3.59 (m, 2H), 3.53-3.46 (m, 1H), 2.61-2.59 (m, 1H), 1.73-1.65 (m, 1H); LCMS: (31.1 +68.3)%, m/z [M+H]⁺=337.1.

### Synthesis of 67.6

To a stirred solution of 67.5 (5.8 g, 17.2 mmol) in MeOH (100 mL) and CH₂Cl₂ (200 mL) was added Pb(OAc)₄ (11.5 g, 25.8 mmol) at 0° C. The resulting reaction mixture was stirred for 30 minutes at 0° C. The reaction mixture was poured into 0.2 M phosphate buffer solution (50 mL) at RT and then filtered through celite bed. The aqueous layer was extracted with CH₂Cl₂ (2×40 mL). The combined organic layer was washed with H₂O (2×50 mL), brine (50 mL), dried over anhydrous Na₂SO₄, filtered and concentrated under vacuum to afford 67.6 (6.0 g) as liquid, which used in the next step without purification. ¹H NMR (500 MHz, CDCl₃): 8.45 (s, 1H), 7.74 (d, J=1.5 Hz, 2H), 7.54-7.42 (m, 7H), 5.22-5.19 (m, 1H).

### Synthesis of 67.7

To a stirred solution of 67.6 (1.5 g, 6.36 mmol) in con. HCl (20 mL) was heated at 110° C. for 16 h. The reaction mixture was cooled to RT and and concentrated under vacuum at 50° C. The resulting residue was triturated with CH₂Cl₂ to afford 67.7 (550 mg) as a pale brown solid. LCMS: 89.0%, m/z [M+H]⁺=236.1.

### Synthesis of 67.8a & 67.8b

To a stirred solution of 66.7 (1.0 g, 4.25 mmol) in EtOH (20 mL) was added 1-(3,5-dichlorophenyl)-1H-pyrrole-2,5-dione (1.09 g, 4.25 mmol), 5,7-dichloroindoline-2,3-dione (0.91 g, 4.25 mmol) at RT. The reaction mixture was heated at 80° C. for 2 h. Then the reaction mixture was concentrated under vacuum to afford residue. The residue was purified by flash chromatography (80 g Silica gel cartridge, gradient elution of 50% EtOAc/pet ether) to afford minor diastereomer 67.8a (170 mg, 6%) as a white solid and major diastereomer 678b (600 mg, 22%) as solid.

67.8a: ¹H NMR (500 MHz, DMSO-d₆): 11.18 (br s, 1H), 7.78 (t, J=2.0 Hz, 1H), 7.64-7.55 (m, 4H), 7.53 (d, J=2.0 Hz, 1H), 7.30 (d, J=1.5 Hz, 1H), 7.25 (d, J=1.5 Hz, 2H), 4.52-4.49 (m, 2H), 3.91 (s, 2H); LCMS: 95.7%, m/z [M−H]⁻=628.1.

67.8b: ¹H NMR (500 MHz, DMSO-d₆): 11.07 (br s, 1H), 7.79 (t, J=2.0 Hz, 1H), 7.63 (d, J=9.0 Hz, 2H), 7.56 (d, J=9.0 Hz, 2H), 7.50 (d, J=2.0 Hz, 1H), 7.30 (d, J=2.0 Hz, 2H), 7.03 (d, J=2.0 Hz, 1H), 5.00-4.92 (m, 1H), 4.32 (d, J=4.5 Hz, 1H), 3.86 (t, J=8.5 Hz, 1H), 3.66 (d, J=6.5 Hz, 1H); LCMS: 99.1%, m/z [M−H]⁻=628.1.

### Synthesis of 67.9a

To a stirred solution of 67.8a (170 mg, 0.269 mmol) in THF (75 mL) was added 1% NaHCO₃ solution (150 mL) at RT. The resulting reaction mixture was stirred for 2 days at RT. After completion of reaction, the pH of solution was adjusted to ˜6-7 with 1N HCl solution and extracted with ethyl acetate (2×40 mL). The combined organic layer was washed with brine (30 mL), dried over anhydrous Na₂SO₄, filtered and concentrated under reduced pressure to afford residue. The residue was purified by prep. HPLC [X-BRIDGE-C18 (150×30) mm, 5 μ; A: 0.1% Formic Acid in H₂O; B: Acetonitrile; Gradient: (Time/%B): 0/50, 8/80, 10/80, 13/98, 14/98, 14.1/50, 17/50 at 25 mL/min] to give 67.9a (12 mg, 7%) as a white solid ¹H NMR (500 MHz, DMSO-d₆): 12.91 (br s, 1H), 11.23 (br s, 1H), 10.92 (br s, 1H), 7.55-7.27 (m, 9H), 4.85-4.68 (m, 1H), 4.25-4.05 (m, 1H), 3.55-3.40 (m, 1H); LCMS: 96.9%, m/z [M−H]⁻=646.1.

### Synthesis of 67.9b

To a stirred solution of 67.8b (400 mg, 0.633 mmol) in THF (150 mL) was added 1% NaHCO₃ solution (300 mL) at RT. The resulting reaction mixture was stirred for 2 days at RT. After completion of reaction, the pH of solution was adjusted to ˜6-7 with 1N HCl solution and extracted with ethyl acetate (2×60 mL). The combined organic layer was washed with brine (60 mL), dried over anhydrous Na₂SO₄, filtered and concentrated under reduced pressure to afford residue. The residue was purified by prep. HPLC [X-BRIDGE-C18 (150×30) mm, 5 μ; A: 0.1% Formic Acid in H₂O; B: Acetonitrile; Gradient: (Time/%B): 0/65, 8/90, 10/90, 10.1/98, 12/98, 12.1/65, 15/65 at 25 mL/min] to give 67.9b (40 mg, 10%) as a white solid. ¹H NMR (500 MHz, DMSO-d₆): 12.48 (br s, 1H), 10.94 (br s, 1H), 10.52 (br s, 1H), 8.18 (d, J=1.5 Hz, 1H), 7.66 (d, J=2.0 Hz, 2H), 7.54-7.45 (m, 5H), 7.30 (br s, 1H), 4.35-4.22 (m, 1H), 3.95-3.86 (m, 1H), 3.72-3.62 (m, 1H), 3.60-3.50 (m, 1H); LCMS: 96.1%, m/z [M−H]⁻=646.1.

TABLE 1

| | SM: | | | |
|---|---|---|---|---|
| Ex- | Isatin (I) | | | |
| am- | Aminoacid (AA) | | | Isolated |
| ple | Maleimide (M) | Compound | M/Z | diastereomers |
| 68 | I: 5,7-dicholoroisatin AA: 2-amino-3-(3-chlorophenyl)- 3,3-difluoropropanoic acid M: 1-(3,5-dichlorophenyl)-1H- pyrrole-2,5-dione | 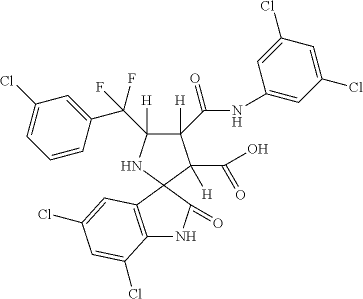 [SMILES: O=C(Nc1cc(Cl)cc(Cl)c1)C1C(C(F)(F)c2cccc(Cl)c2)NC2(C(=O)Nc3c(Cl)cc(Cl)cc32)C1C(=O)O] | 646.1 [M − H]⁻ 646.1 [M − H]⁻ 646.1 [M − H]⁻ | 68.a 68.b 68.c |
| 69 | I: 5,7-dicholoroisatin AA: 2-amino-3,3-difluoro-3-(2- (trifluoromethyl)phenyl)propanoic acid M: 1-(3,5-dichlorophenyl)-1H- pyrrole-2,5-dione | 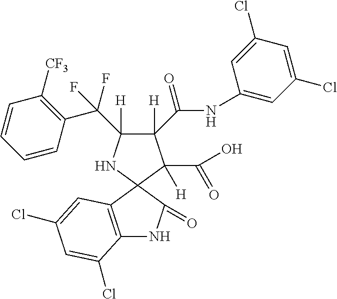 [SMILES: O=C(Nc1cc(Cl)cc(Cl)c1)C1C(C(F)(F)c2ccccc2C(F)(F)F)NC2(C(=O)Nc3c(Cl)cc(Cl)cc32)C1C(=O)O] | 680.1 [M − H]⁻ 681.9 [M + H]⁺ | 69.a 69.b |
| 70 | I: 5,7-dicholoroisatin AA: 2-amino-3,3-difluoro-3-(3- (trifluoromethyl)phenyl)propanoic acid M: 1-(3,5-dichlorophenyl)-1H- pyrrole-2,5-dione | 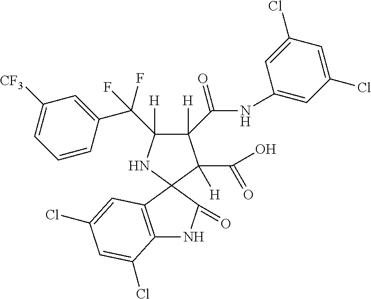 [SMILES: O=C(Nc1cc(Cl)cc(Cl)c1)C1C(C(F)(F)c2cccc(C(F)(F)F)c2)NC2(C(=O)Nc3c(Cl)cc(Cl)cc32)C1C(=O)O] | 680.0 [M − H]⁻ 679.9 [M − H]⁻ 682.1 [M + H]⁺ | 70.a 70.b 70.c |
| 71 | I: 5,7-dicholoroisatin AA: 2-amino-3,3-difluoro-3-(p- tolyl)propanoic acid M: 1-(3,5-dichlorophenyl)-1H- pyrrole-2,5-dione | 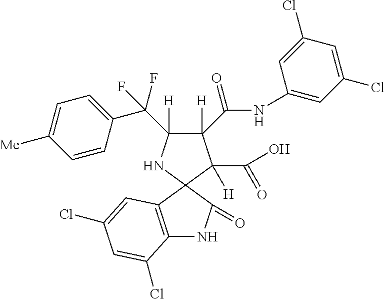 [SMILES: Cc1ccc(C(F)(F)C2NC3(C(=O)Nc4c(Cl)cc(Cl)cc43)C(C(=O)O)C2C(=O)Nc2cc(Cl)cc(Cl)c2)cc1] | 628.1 [M + H]+ | 71.a Major diastereomer only collected |
| 72 | I: 5,7-dicholoroisatin AA: 2-amino-3,3-difluoro-3- (pyridin-2-yl)propanoic acid M: 1-(3,5-dichlorophenyl)-1H- pyrrole-2,5-dione | 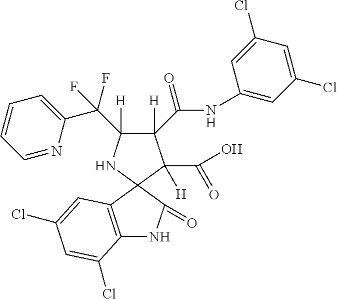 [SMILES: O=C(Nc1cc(Cl)cc(Cl)c1)C1C(C(F)(F)c2ccccn2)NC2(C(=O)Nc3c(Cl)cc(Cl)cc32)C1C(=O)O] | 615.1 [M + H]⁺ 615.1 [M + H]⁺ 614.9 [M + H]⁺ | 72.a 72.b 72.c |
| 73 | I: 5,7-dicholoroisatin AA: 2-amino-3,3-difluoro-3-(4- (trifluoromethyflphenyppropanoic acid M: 1-(3,5-dichlorophenyl)-1H- pyrrole-2,5-dione | 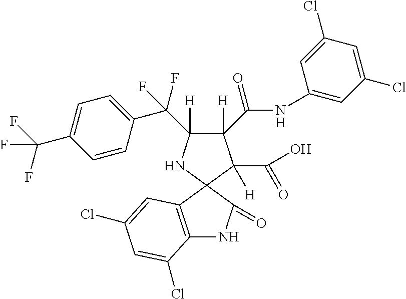 [SMILES: O=C(Nc1cc(Cl)cc(Cl)c1)C1C(C(F)(F)c2ccc(C(F)(F)F)cc2)NC2(C(=O)Nc3c(Cl)cc(Cl)cc32)C1C(=O)O] | 682.1 [M + H]⁺ 682.1 [M + H]⁺ 682.1 [M + H]⁺ | 73.a 73.b 73.c |
| 74 | I: 5,7-dicholoroisatin AA: 2-amino-3,3-difluoro-3- (pyridin-3-yl)propanoic acid M: 1-(3,5-dichlorophenyl)-1H- pyrrole-2,5-dione | 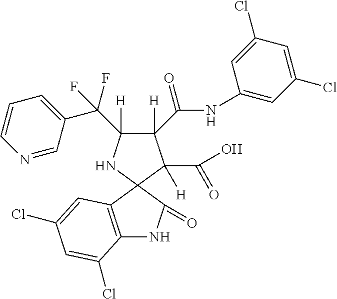 [SMILES: O=C(Nc1cc(Cl)cc(Cl)c1)C1C(C(F)(F)c2cccnc2)NC2(C(=O)Nc3c(Cl)cc(Cl)cc32)C1C(=O)O] | 614.1 [M + H]+ | 74.a Major diastereomer only collected |
| 75 | I: I: 5,7-dicholoroisatin AA: (4S)-4-hydroxy-4- (trifluoromethyl)pyrrolidine-2- carboxylic acid M: 1-(6-(trifluoromethyl)pyrazin- 2-yl)-1H-pyrrole-2,5-dione | 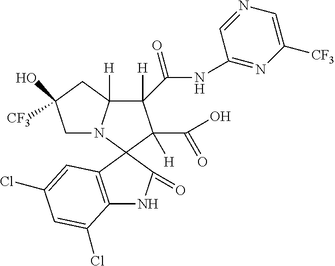 [SMILES: O=C(Nc1cncc(C(F)(F)F)n1)C1C2C[C@@](O)(C(F)(F)F)CN2C2(C(=O)Nc3c(Cl)cc(Cl)cc32)C1C(=O)O] | 612.2 [M − H]− | 75.a Isolated as single enantiomer resulting from chiral AA |
| 76 | I: 5,7-dicholoroisatin AA: 4,4-difluoropyrrolidine-2- carboxylic acid M: 1-(3-hydroxyphenyl)-1H- pyrrole-2,5-dione | 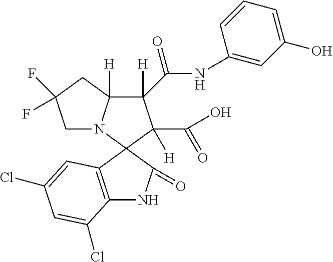 [SMILES: O=C(Nc1cccc(O)c1)C1C2CC(F)(F)CN2C2(C(=O)Nc3c(Cl)cc(Cl)cc32)C1C(=O)O] | 512.1 [M + H]⁺ 512.1 [M + H]⁺ 512.1 [M + H]⁺ 512.1 [M + H]⁺ | 76.a 76.b 76.c 76.d |
| 77 | I: 5,7-dicholoroisatin AA: 4,4-difluoropyrrolidine-2- carboxylic acid M: 1-(4-hydroxyphenyl)-1H- pyrrole-2,5-dione | 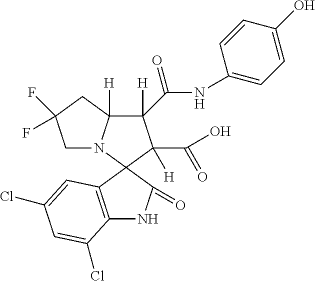 [SMILES: O=C(Nc1ccc(O)cc1)C1C2CC(F)(F)CN2C2(C(=O)Nc3c(Cl)cc(Cl)cc32)C1C(=O)O] | 512.0 [M + H]⁺ 512.0 [M + H]⁺ | 77.a 77.b |
| 78 | I: 5,7-dicholoroisatin AA: 4,4-difluoropyrrolidine-2- carboxylic acid M: 1-(2-hydroxyphenyl)-1H- pyrrole-2,5-dione | 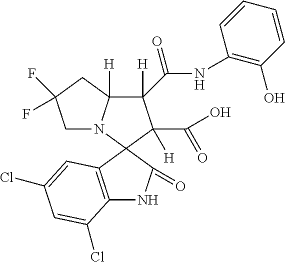 [SMILES: O=C(Nc1ccccc1O)C1C2CC(F)(F)CN2C2(C(=O)Nc3c(Cl)cc(Cl)cc32)C1C(=O)O] | 512.1 [M + H]⁺ | 78.a Major diastereomer only collected |
| 79 | I: 5,7-dicholoroisatin AA: (2,2,2-trifluoroethyl)glycine M: 1-(4-hydroxyphenyl)-1H- pyrrole-2,5-dione | 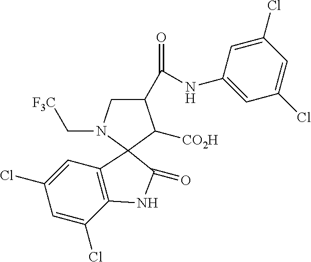 [SMILES: O=C(Nc1cc(Cl)cc(Cl)c1)C1CN(CC(F)(F)F)C2(C(=O)Nc3c(Cl)cc(Cl)cc32)C1C(=O)O] | 567.9 [M − H]− 567.9 [M − H]− 567.9 [M − H]− | 79a 79b 79c |

Following Examples 66 and 67, the following diastereomeric examples were made. Depending on substituents on the corresponding analogous intermediates 66.8a or 66.8b, epimerized products might be found or in the case of corresponding analogous intermediates 67.8a or 67.8b, the desired products were isolated without epimerization.

### Example 80

### Synthesis of 5,7-dichloro-6′,6′-difluoro-1′-((6-methoxy-4-(trifluoromethyl)pyridin-2-yl)carbamoyl)-2-oxo-1′,2′,5′,6′,7′,7a′-hexahydrospiro[indoline-3,3′-pyrrolizine]-2′-carboxylic acid, 80.7a.1, 80.7a.2, 80.7b.1 & 80.7b.2

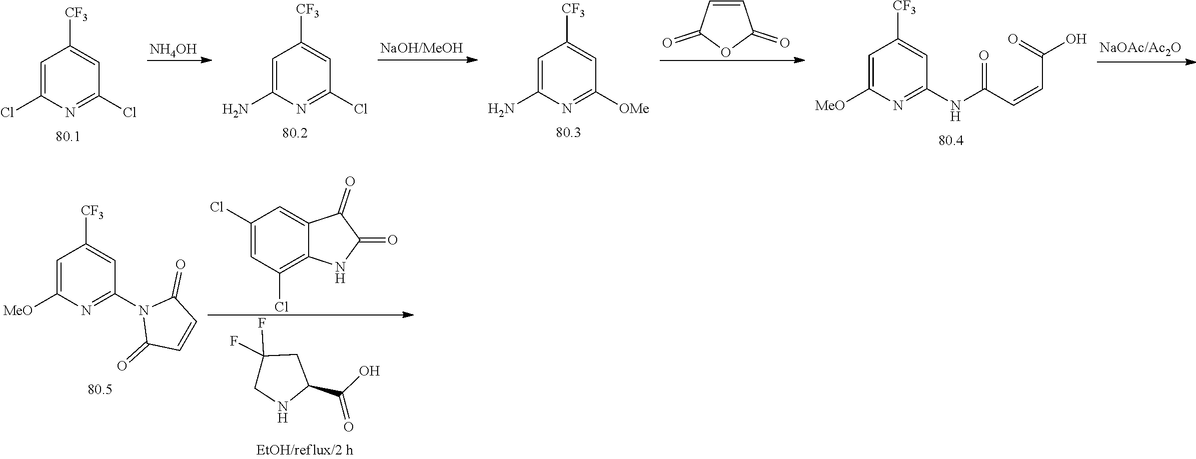

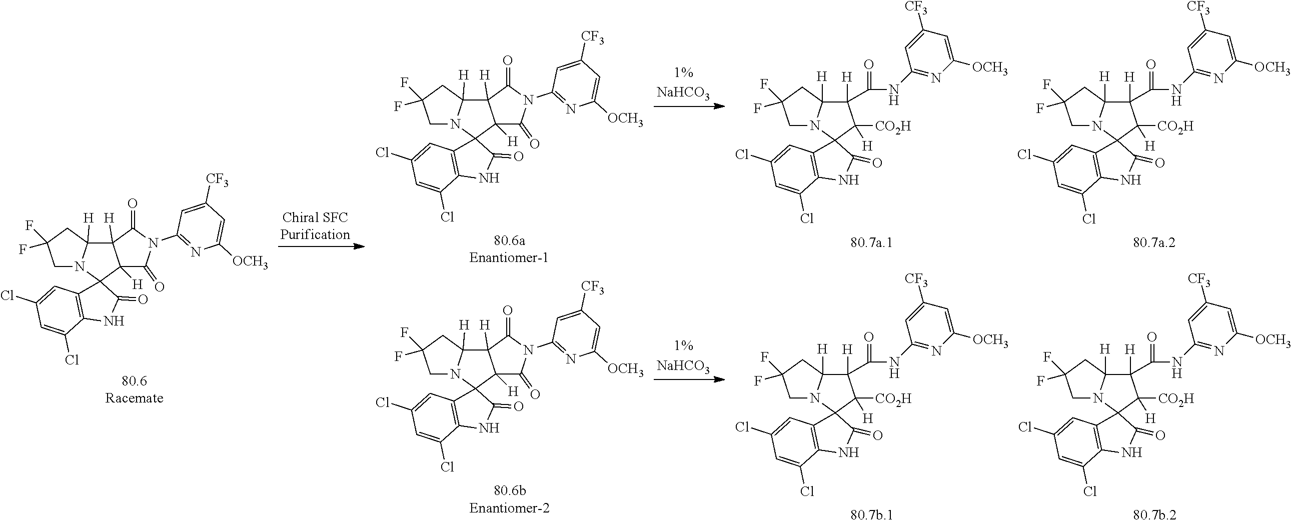

### Synthesis of 80.2

A solution of 80.1 (15 g, 69.4 mmol) in NH₄OH (150 mL) was stirred in a steel bomb at 180° C. for 16 h. The reaction mixture was cooled to RT, diluted with H₂O and extracted with EtOAc (3×200 mL). The combined organic layer was concentrated under reduced pressure. The residue was purified by column chromatography (Silica gel 100-200 mesh, 20% EtOAc in pet ether) to afford 80.2 (7 g, 51%) as solid.

¹HNMR (400 MHz, CDCl₃): 6.85 (s, 1H), 6.58 (s, 1H), 4.96 (br s, 2H); LCMS: 76.1%, m/z [M+H]⁺=197.0.

### Synthesis of 80.3

80.2 (12 g, 61.0 mmol) was dissolved in 25% NaOH in MeOH (120 mL) solution. After stirring in a steel bomb for 16 h at 100° C., the reaction mixture was cooled to RT, diluted with H₂O and extracted with EtOAc (3×200 mL). The combined organic layer was concentrated under vacuum. The residue was purified by column chromatography (Silica gel 100-200 mesh, 20% EtOAc in pet ether) to afford 80.3 (9.8 g, 83%) as solid.

¹H NMR (400 MHz, CDCl₃): 6.28 (s, 1H), 6.23 (s, 1H), 4.56 (br s, 2H), 3.87 (s, 3H); LCMS: 88.1%, m/z [M+H]⁺=193.1.

Synthesis of 80.4:

To a stirred solution of furan-2,5-dione (4.49 g, 45.8 mmol) in MTBE (100 mL) was added 80.3 (8.8 g, 45.8 mmol) at RT. After stirring for 16 h at RT, the precipitated solid was filtered and dried under vacuum. The residue was washed with MTBE to afford 80.4 (13.0 g, 66%) as a solid.

¹H NMR (500 MHz, CDCl₃): 8.46 (d, J=6.5 Hz, 1H), 8.03 (s, 1H), 6.87 (s, 1H), 6.54 (d, J=12.5 Hz, 1H), 6.41 (d, J=12.5 Hz, 1H), 3.93 (s, 3H); LCMS: 98.4%, m/z [M+H]⁺=291.0.

### Synthesis of 80.5

To a stirred solution of 80.4 (5.0 g, 19.2 mmol) in Ac₂O (50 mL) was added NaOAc (1.57 g, 19.2 mmol) at RT. After stirring at 80° C. for 2 h, the reaction mixture was cooled to RT and the excess Ac₂O was evaporated under reduced pressure. The residue was diluted with DCM (50 mL) and washed with water (2×25 mL). The combined organic layer was dried over Na₂SO₄, filtered and concentrated. The residue was purified by flash column chromatography (80 g Silica gel cartridge, 20% EtOAc in pet ether) to afford 80.5 (3.0 g, 64%) as a solid.

¹H NMR (400 MHz, CDCl₃): 7.14 (s, 1H), 6.99 (s, 1H), 6.90 (s, 2H), 3.97 (s, 3H); LCMS: 98.1%, m/z [M+H]⁺=273.0.

### Synthesis of 80.6

To a stirred solution of 80.5 (2.5 g, 9.19 mmol) in EtOH (30 mL) was added 5,7-dichloroindoline-2,3-dione (1.98 g, 9.19 mmol), (S)-4,4-difluoropyrrolidine-2-carboxylic acid (2.27 g, 9.19 mmol) at RT. After stirring at 80° C. for 2 h, the reaction mixture was concentrated under reduced pressure. The residue was purified by flash chromatography (120 g Silica gel cartridge, 30% EtOAc/pet ether) to afford major diastereomer 80.6 (4.4 g, 84%) as a yellow solid.

¹H NMR (500 MHz, DMSO-d₆): 11.40 (s, 1H), 7.59 (d, J=2.0 Hz, 1H), 7.46 (s, 1H), 7.39 (d, J=1.0 Hz, 1H), 7.14 (d, J=1.5 Hz, 1H), 4.44-4.39 (m, 1H), 4.02 (s, 3H), 3.99-3.91 (m, 1H), 3.90 (t, J=7.5 Hz, 1H), 3.09-3.05 (m, 1H), 2.69-2.63 (m, 1H), 2.57-2.51 (m, 1H), 2.36-2.30 (m, 1H); LCMS: 96.9%, m/z [M−H]⁻=574.8; Chiral purity: (46.6+47.9)%. Separation of 80.6a & 80.6b (absolute stereochemistry of Enantiomer 1 & 2 not determined):

80.6 (4.4 g) was purified by chiral SFC using Chiral pack IG (250×30) mm, 5 μ; 0.2% TFA in n-Hexane: Isopropanol (85:15) at RT (Isocratic 42.0 mL/min, 13 min run time with detection at 254 nm). Pure fractions were concentrated under reduced pressure to give 480 mg of 80.6a (Enantiomer-1) as a yellow solid and 470 mg of 80.6b (Enantiomer-2) as a yellow solid.

80.6a:¹H NMR (500 MHz, DMSO-d₆): 11.40 (s, 1H), 7.59 (d, J=2.0 Hz, 1H), 7.46 (s, 1H), 7.39 (d, J=0.5 Hz, 1H), 7.14 (d, J=2.0 Hz, 1H), 4.44-4.39 (m, 1H), 4.02 (s, 3H), 3.98 (d, J=7.5 Hz, 1H), 3.90 (t, J=7.5 Hz, 1H), 3.09-3.07 (m, 1H), 2.71-2.65 (m, 1H), 2.60-2.51 (m, 1H), 2.36-2.29 (m, 1H); LCMS: 99.5%, m/z [M+H]⁺=576.9; Chiral purity: 98.0%.

80.6b:'H NMR (500 MHz, DMSO-d₆): 11.04 (s, 1H), 7.59 (d, J=2.0 Hz, 1H), 7.46 (s, 1H), 7.39 (s, 1H), 7.14 (d, J=1.5 Hz, 1H), 4.44-4.39 (m, 1H), 4.02 (s, 3H), 3.98 (d, J=7.5 Hz, 1H), 3.90 (t, J=7.5 Hz, 1H), 3.11-3.05 (m, 1H), 2.69-2.63 (m, 1H), 2.57-2.51 (m, 1H), 2.35-2.30 (m, 1H); LCMS: 98.6%, m/z [M+H]⁺=576.9; Chiral purity: 99.9%.

### Synthesis of 80.7a.1 & 80.7a.2

To a stirred solution of 80.6a (300 mg, 0.52 mmol) in THF (100 mL) was added 1% NaHCO₃ solution (200 mL) at RT. The resulting reaction mixture was stirred for 16 h at RT. After completion of reaction, the pH of the solution was adjusted to −6-7 with IN HCl solution and extracted with EtOAc (2×50 mL). The combined organic layer was washed with brine (60 mL), dried over anhydrous Na₂SO₄, filtered and concentrated under reduced pressure to afford residue. The residue was purified by prep. HPLC [X-BRIDGE C₁₈ (150×30) mm, 5 μ; A: 0.1% Formic acid in H₂O, B: Acetonitrile; Gradient: (Time/%B): 0/40, 8/80, 9/80, 9.1/98, 14/98, 14.1/40, 17/40 at 25 mL/min] to afford 80.7a.1 (11 mg) as a white solid and 80.7a.2 (208 mg) as a white solid.

80.7a.1: ¹HNMR (500 MHz, DMSO-d₆): 12.74 (br s, 1H), 11.11 (br s, 1H), 7.83 (s, 1H), 7.49 (s, 1H), 7.05 (br s, 1H), 6.88 (s, 1H), 4.25-4.15 (m, 2H), 3.83 (s, 3H), 3.78 (d, J=7.0 Hz, 1H), 3.55-3.50 (m, 1H), 3.24-3.15 (m, 1H), 2.85-7.73 (m, 1H), 2.40-2.30 (m, 1H); LCMS: 98.6%, m/z [M+H]⁺=595.2.

80.7a.2: ¹HNMR (500 MHz, DMSO-d₆): 12.49 (br s, 1H), 11.08 (br s, 1H), 11.04 (br s, 1H), 8.05 (s, 1H), 8.02 (s, 1H), 7.48 (d, J=1.5 Hz, 1H), 6.92 (s, 1H), 4.08-4.04 (m, 2H), 3.94 (s, 3H), 3.85-3.78 (m, 1H), 3.17-3.14 (m, 1H), 2.67-2.63 (m, 1H), 2.50-2.44 (m, 1H), 2.08-1.90 (m, 1H); LCMS: 98.2%, m/z [M+H]⁺=595.2.

### Synthesis of 80.7b.1 & 80.7b.2

To a stirred solution of 80.6b (300 mg, 0.519 mmol) in THF (100 mL) was added 1% NaHCO₃ solution (200 mL) at RT. The resulting reaction mixture was stirred for 16 h at RT. After completion of reaction, the pH of the solution was adjusted to ˜6-7 with 1N HCl solution and extracted with EtOAc (2×50 mL). The combined organic layer was washed with brine (60 mL), dried over anhydrous Na₂SO₄, filtered and concentrated under reduced pressure to afford residue. The residue was purified by prep. HPLC [X-BRIDGE C₁₈ (150×30) mm, 5 μ; A: 0.1% Formic acid in H₂O, B: Acetonitrile; Gradient: (Time/%B): 0/50, 9/80, 11/80, 11.1/98, 12/98, 12.1/50, 15/50 at 25 mL/min] to afford 80.7b.1 (13 mg) as a white solid and 80.7b.2 (135 mg) as a white solid.

80.7b.1: ¹H NMR (500 MHz, DMSO-d₆): 12.76 (br s, 1H), 11.12 (br s, 1H), 7.83 (s, 1H), 7.49 (s, 1H), 7.06 (br s, 1H), 6.89 (s, 1H), 4.26-4.05 (m, 2H), 3.83 (s, 3H), 3.78 (d, J=8.0 Hz, 1H), 3.55-3.49 (m, 1H), 3.28-3.11 (m, 1H), 2.82-2.77 (m, 1H), 2.38-2.36 (m, 1H); LCMS: 98.6%, m/z [M+H]⁺=595.2.

80.7b.2: ¹H NMR (500 MHz, DMSO-d₆): 12.50 (br s, 1H), 11.08 (br s, 1H), 11.02 (br s, 1H), 8.06 (br s, 1H), 8.02 (s, 1H), 7.49 (d, J=1.5 Hz, 1H), 6.92 (s, 1H), 4.09-4.04 (m, 2H), 3.94 (s, 3H), 3.85-3.78 (m, 1H), 3.19-3.12 (m, 1H), 2.69-2.62 (m, 1H), 2.50-2.40 (m, 1H), 2.08-1.90 (m, 1H); LCMS: 98.0%, m/z [M+H]⁺=595.2.

### Example 81

### Synthesis of 5,7-dichloro-4′4(3,5-dichlorophenyl)carbamoyl)-1-methyl-2-oxo-5′-(trifluoromethyl)spiro[indoline-3,2′-pyrrolidinel-3′-carboxylic acid

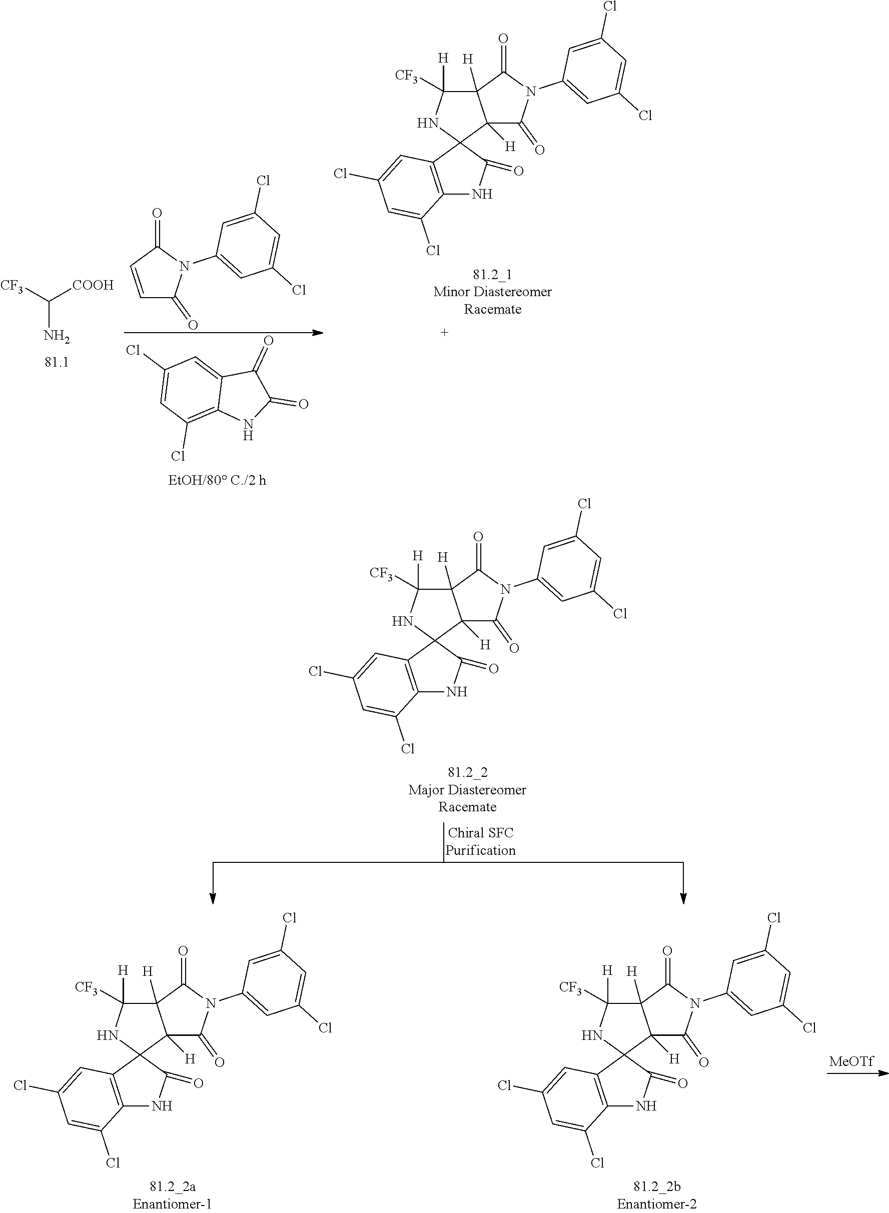

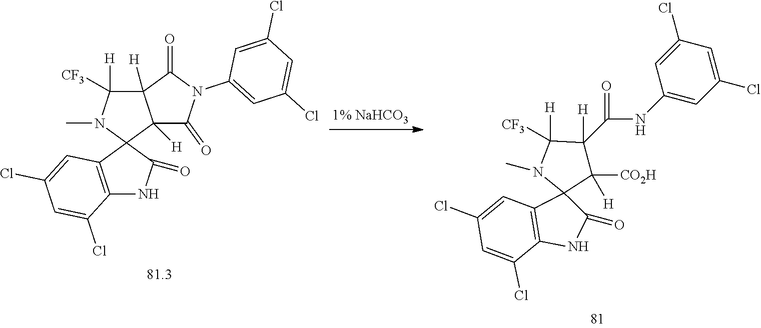 [SMILES: CN1C(C(F)(F)F)C2C(=O)N(c3cc(Cl)cc(Cl)c3)C(=O)C2C12C(=O)Nc1c(Cl)cc(Cl)cc12 ; CN1C(C(F)(F)F)C(C(=O)Nc2cc(Cl)cc(Cl)c2)C(C(=O)O)C12C(=O)Nc1c(Cl)cc(Cl)cc12]

### Synthesis of 81.2_1 and 81.2_2

To a stirred solution of 81.1 (1 g, 6.98 mmol) in EtOH (40 mL) were added 1-(3,5-dichlorophenyl)-1H-pyrrole-2,5-dione (1.69 g, 6.98 mmol) and 5,7-dichloroindoline-2,3-dione (1.50 g, 6.98 mmol). After stirring for 3 h at 80° C., the reaction mixture was cooled to RT and concentrated. The residue was purified by flash chromatography (40 g Silica gel cartridge, 10% EtOAc in pet ether) to afford minor diastereomer 81.2_1 (400 mg, 10%) as a solid and major diastereomer 81.2_2 (2.2 g, 59%) as a solid.

81.2_1: ¹H NMR (400 MHz, DMSO-d₆): 11.28 (br s, 1H), 7.78 (t, J=2.0 Hz, 1H), 7.54 (d, J=2.0 Hz, 1H), 7.48 (d, J=2.0 Hz, 1H), 7.26 (d, J=2.0 Hz, 2H), 4.88 (d, J=5.2 Hz, 1H), 4.50-4.46 (m, 1H), 4.12 (d, J=10.0 Hz, 1H), 3.95-3.92 (m, 1H); LCMS: 98.8%, m/z [M-H]⁻=535.9.

81.2_2: ¹H NMR (400 MHz, DMSO-d₆): 11.17 (br s, 1H), 7.79 (t, J=2.0 Hz, 1H), 7.53 (d, J=2.0 Hz, 1H), 7.38 (d, J=2.0 Hz, 2H), 7.07 (d, J=2.0 Hz, 1H), 4.88-4.83 (m, 2H), 4.06-4.01 (m, 1H), 3.71 (d, J=8.4 Hz, 1H); LCMS: 98.3%, m/z [M-H]⁻=535.9.

### Separation of 81.2_2a & 81.2_2b

81.2_2 (2.2 g) was purified by chiral SFC using (R, R) Whelk-01 (30×250 mm), 5 μ; 80% CO₂: 20% acetonitrile at RT (Isocratic 90 g/min, with detection at 214 nm) to give 81.2_2a (Enantiomer-1, 900 mg, 82%) as a solid and 81.2_2b (Enantiomer-2, 850 mg, 77%) as a solid. (absolute stereochemistry of Enantiomer 1 & 2 were not determined)

81.2_2a: ¹H NMR (400 MHz, DMSO-d₆): 11.17 (br s, 1H), 7.80 (t, J=2.0 Hz, 1H), 7.54 (d, J=2.0 Hz, 1H), 7.38 (d, J=2.0 Hz, 2H), 7.07 (d, J=2.0 Hz, 1H), 4.91-4.84 (m, 2H), 4.05-4.01 (m, 1H), 3.71 (d, J=8.0 Hz, 1H); LCMS: 99.4%, m/z [M−H]⁻=535.9. 81.2_2b: ¹H NMR (400 MHz, DMSO-d₆): 11.18 (br s, 1H), 7.80 (t, J=2.0 Hz, 1H), 7.53 (d, J=2.0 Hz, 1H), 7.38 (d, J=2.0 Hz, 2H), 7.07 (d, J=1.6 Hz, 1H), 4.89-4.84 (m, 2H), 4.05-4.01 (m, 1H), 3.71 (d, J=8.0 Hz, 1H); LCMS: 99.3%, m/z [M−H]⁻=535.9.

### Synthesis of 81.3

To a stirred solution of 81.2_2b (200 mg, 0.37 mmol) in DCM (5 mL) was added TEA (0.3 mL, 2.14 mmol) followed by Methyltriflate (0.3 mL, 2.74 mmol) at 0° C. After stirring for 16 h at RT, the reaction mixture was quenched with ice cold water and extracted with EtOAc (2×20 mL). The combined organic layer was dried over Na₂SO₄, filtered and concentrated under vacuum pressure. The residue was purified by prep. HPLC [Column: INERTSIL-ODS 2 (250×19mm), 5 μ; A: 0.1% Formic Acid in H₂O, B: Acetonitrile; Gradient:(T%B) 0/55, 8/80, 11/90, 11.1/98, 13/98, 13.1/55, 17/55 at 18 mL/min] to afford 81.3 (14 mg, 7%) as a white solid.

¹H NMR (500 MHz, DMSO-d₆): 11.51 (br s, 1H), 7.80 (t, J=2.0 Hz, 1H), 7.59 (d, J=1.5 Hz, 1H), 7.40 (d, J=1.5 Hz, 2H), 7.10 (d, J=1.5 Hz, 1H), 4.57-4.54 (m, 1H), 4.11 (t, J=9.0 Hz, 1H), 3.78 (d, J=8.5 Hz, 1H), 2.13 (s, 3H); LCMS: 98.7%, m/z [M−H]⁻=549.9.

### Synthesis of 81

To a stirred solution of 81.3 (30 mg, 0.054 mmol) in THF (20 mL) was added 1% NaHCO₃ solution (41 mL) at RT. After stirring for 16 h at RT, the pH of the solution was adjusted to ˜6-7 with 1N HCl solution and extracted with EtOAc (2×10 mL). The combined organic layer was washed with brine (10 mL), dried over anhydrous Na₂SO₄, filtered and concentrated under reduced pressure. The residue was purified by prep. HPLC [Column: SYMMETRY-C8 (300×19 mm),7 μ; A: 0.1% Formic Acid in H₂O, B: Acetonitrile; Gradient:(T%B): -0/65, 8/90, 10/90, 10.1/98, 12/98, 12.1/65, 16/65 at 18 mL/min] to afford 81 (6 mg, 19%) as a solid.

¹H NMR (500 MHz, DMSO-d₆): 12.61 (br s, 1H), 11.02 (br s, 1H), 10.70 (s, 1H), 8.22 (s, 1H), 7.67 (d, J=1.5 Hz, 2H), 7.39 (s, 1H), 7.27 (s, 1H), 4.13-4.03 (m, 1H), 3.81-3.70 (m, 1H), 3.62-3.55 (m, 1H), 2.05 (s, 3H); LCMS: 98.7%, m/z [M−H]⁻=567.9.

### Example 82

### Synthesis of (1′R,2′S,7a′R)-5,7-dichloro-1-((3,5-dichlorophenyl)(neopentyl)carbamoyl)-2-oxo-1′,2′,5′,6′,7′,7a′-hexahydrospiro[indoline-3,3′-pyrrolizine]-2′-carboxylic acid (82.6a) and (1′S,2′R,7a′S)-5,7-dichloro-1-((3,5-dichlorophenyl)(neopentyl)carbamoyl)-2-oxo-1′,2′,5′,6′,7′,7a′-hexahydrospiro[indoline-3,3′-pyrrolizine]-2′-carboxylic acid, (82.6b)

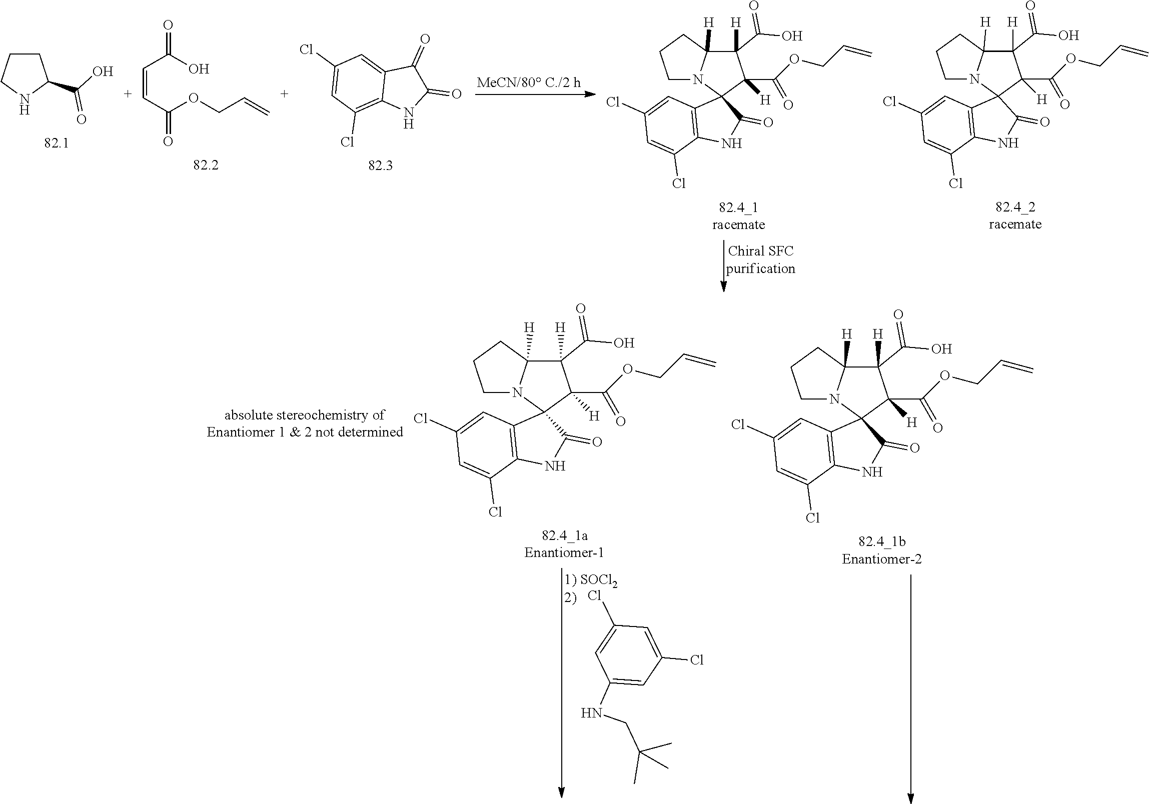

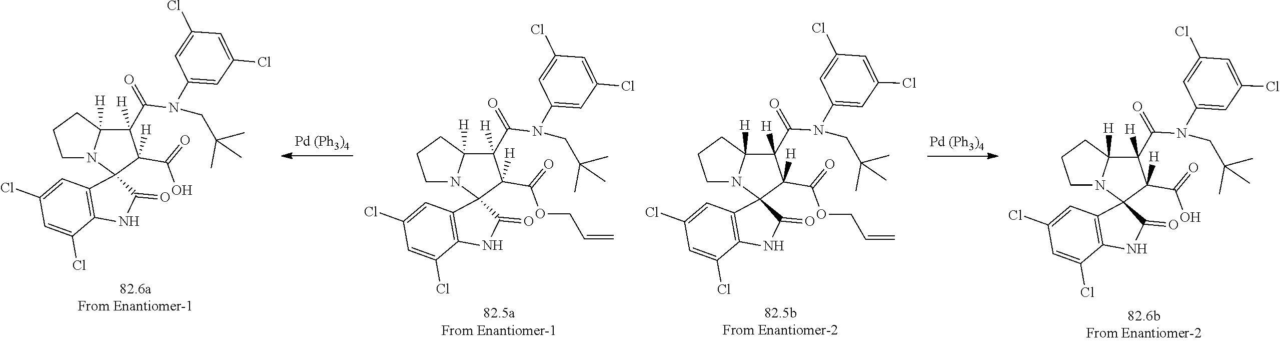 [SMILES: CC(C)(C)CN(C(=O)[C@H]1[C@@H](C(=O)O)[C@]2(C(=O)Nc3c(Cl)cc(Cl)cc32)N2CCC[C@@H]12)c1cc(Cl)cc(Cl)c1 ; C=CCOC(=O)[C@@H]1[C@H](C(=O)N(CC(C)(C)C)c2cc(Cl)cc(Cl)c2)[C@@H]2CCCN2[C@@]12C(=O)Nc1c(Cl)cc(Cl)cc12 ; C=CCOC(=O)[C@H]1[C@@H](C(=O)N(CC(C)(C)C)c2cc(Cl)cc(Cl)c2)[C@H]2CCCN2[C@]12C(=O)Nc1c(Cl)cc(Cl)cc12 ; CC(C)(C)CN(C(=O)[C@H]1[C@H]2CCCN2[C@]2(C(=O)Nc3c(Cl)cc(Cl)cc32)[C@H]1C(=O)O)c1cc(Cl)cc(Cl)c1]

### Synthesis of 82.4_1 & 82.4_2

To a solution of (S)-pyrrolidine-2-carboxylic acid, 82.1, (10 g, 66.2 mmol) in MeCN (100 mL) was added (Z)-4-(allyloxy)-4-oxobut-2-enoic acid, 82.2, (10.3 g, 66.2 mmol) and 5,7-dichloroindoline-2,3-dione, 82.3, (14.3 g, 66.2 mmol) at RT. After refluxing for 2 h, the reaction mixture was cooled to RT. The resulting precipitate was filtered and washed with MeCN (2×20 mL) and dried under high vacuum to give 82.4_1 & 82.4_2 (LCMS ratio: 26:35).

82.4_1: rac-(1′R,2′S,3R,7a′R)-2′-((allyloxy)carbonyl)-5,7-dichloro-2-oxo-1′,2′,5′,6′,7′,7a′-hexahydrospiro[indoline-3,3′-pyrrolizine]-1′-carboxylic acid. ¹H NMR (500 MHz, DMSO-d₆): 12.56 (br s, 1H), 11.00 (s, 1H), 7.77 (d, J=2.0 Hz, 1H), 7.46 (d, J=2.0 Hz, 1H), 5.50-5.45 (m, 1H), 5.10-5.05 (m, 2H), 4.26 (d, J=5.5 Hz, 2H), 4.06 (d, J=7.5 Hz, 1H), 3.97-3.96 (m, 1H), 3.55-3.52 (m, 1H), 2.64-2.62 (m, 1H), 2.26-2.23 (m, 1H), 1.91-1.78 (m, 3H), 1.53-1.49 (m, 1H); LCMS: 94.0%, m/z [M+H]⁺=425.0; Chiral purity: (49.7 +50.2)%. Regiochemistry and relative stereochemistry was confirmed by 2D NMR studies. 82.4_2: ¹H NMR (500 MHz, DMSO-d₆): 12.40 (br s, 1H), 10.95 (s, 1H), 7.56 (d, J=2.0 Hz, 1H), 7.51 (d, J=2.0 Hz, 1H), 5.86-5.80 (m, 1H), 5.27-5.15 (m, 2H), 4.45-4.39 (m, 2H), 4.27-4.25 (m, 1H), 3.64 (d, J=8.5 Hz, 1H), 3.47-3.44 (m, 1H), 2.51-2.48 (m, 1H), 2.42-2.35 (m, 1H), 2.10-2.00 (m, 1H), 1.90-1.80 (m, 1H), 1.73-1.71 (m, 2H); LCMS: 80.7%, m/z [M+H]⁺=425.0. Unknown relative regiochemistry.

### Separation of 82.4_la & 82.4_1b

82.4_1 (10 g) was separated by chiral SFC using Chiral pack IG (4.6×250) mm, 5 μ; 0.5% TFA in Isopropanol at RT (Isocratic 42.0 mL/min, 16 min run time with detection at 214 nm) to give 1.8 g of 82.4_la (Peak-1) as a white solid and 3.8 g of 82.4_1b (Peak-2) as a solid. (absolute stereochemistry of Enantiomer 1 & 2 not determined).

82.4_1a: ¹H NMR (500 MHz, DMSO-d₆): 12.55 (br s, 1H), 11.00 (s, 1H), 7.77 (d, J=2.0 Hz, 1H), 7.46 (d, J=2.0 Hz, 1H), 5.50-5.45 (m, 1H), 5.10-5.05 (m, 2H), 4.26 (d, J=5.5 Hz, 2H), 4.06 (d, J=8.0 Hz, 1H), 3.97-3.96 (m, 1H), 3.54-3.51 (m, 1H), 2.64-2.62 (m, 1H), 2.26-2.23 (m, 1H), 1.91-1.80 (m, 3H), 1.53-1.49 (m, 1H); LCMS: 99.0%, m/z [M+H]⁺=425.0; Chiral purity: 99.9%.

82.4_1b: ¹H NMR (400 MHz, DMSO-d₆): 12.58 (br s, 1H), 11.04 (s, 1H), 7.76 (s, 1H), 7.47 (d, J=1.6 Hz, 1H), 5.53-5.43 (m, 1H), 5.11-5.05 (m, 2H), 4.27 (d, J=5.2 Hz, 2H), 4.07 (d, J=7.6 Hz, 1H), 4.04-3.97 (m, 1H), 3.58-3.54 (m, 1H), 2.70-2.65 (m, 1H), 2.30-2.27 (m, 1H), 1.93-1.75 (m, 3H), 1.59-1.52 (m, 1H); LCMS: 97.2%, m/z [M+H]⁺=425.0; Chiral purity: 97.7%.

### Synthesis of 82.5a

Thionyl chloride (6 mL) was added to 82.4_la (300 mg, 0.70 mmol) at RT. After stirring for 2 h at RT, the reaction mixture was concentrated under reduced pressure. To the resulting acid chloride in CH₂Cl₂ (3 mL) was added a solution of 3,5-dichloro-N-neopentylaniline (245 mg, 1.05 mmol) in CH₂Cl₂ (2 mL). After stirring for 16 h at 55° C., the reaction mixture was quenched with water (20 mL) and extracted with CH₂Cl₂ (2×20 mL). The combined organic layer was washed with brine, dried over anhydrous Na₂SO₄, filtered and concentrated under reduced pressure. The residue was purified by column chromatography (40 g Silica gel cartridge, 20% EtOAc/pet ether) to afford 82.5a (200 mg, 50%) as a solid.

¹H NMR (400 MHz, DMSO-d₆) (Exist in rotameric form): 10.97/10.92 (s, 1H), 8.22/7.70 (d, J=2.0 Hz, 1H), 7.64 (d, J=1.2 Hz, 1H), 7.54-7.40 (m, 3H), 5.52-5.43 (m, 1H), 5.21-5.08 (m, 2H), 4.34-4.28 (m, 1H), 4.22-4.15 (m, 2H), 3.83-3.77 (m, 1H), 3.71-3.58 (m, 3H), 2.70-2.64 (m, 1H), 2.20-2.14 (m, 1H), 1.97-1.83 (m, 1H), 1.81-1.73 (m, 2H), 1.57-1.50 (m, 1H), 0.89-0.78 (m, 9H); LCMS: 98.8%, m/z [M+H]⁺=638.0.

Synthesis of 82.6a

To a stirred solution of 82.5a (200 mg, 0.31 mmol) in THF (4 mL) were added aniline (30 mg, 0.31 mmol) and Pd(PPh₃)₄ (72 mg, 0.06 mmol) at RT. After stirring for 2 h at RT, the reaction mixture was concentrated under reduced pressure. The residue was purified by prep. HPLC [KROMOSIL-C18 (150×25) mm, 10 μ; A: 0.1% Formic acid in H₂O, B: Acetonitrile; Gradient: (Time in minutes/%B): 0/70, 8/90, 11/95, 11.1/98, 13/98, 13.1/70, 15/70 at 20 mL/min] to afford 82.6a (58 mg, 37%) as a solid. ¹H NMR (500 MHz, DMSO-d₆) (Exist in rotameric form): 12.44 (br s, 1H), 10.90/10.84 (s, 1H), 8.37/7.86 (d, J=2.0 Hz, 1H), 7.62-7.61 (m, 1H), 7.53-7.38 (m, 3H), 4.08 (d, J=8.0 Hz, 1H), 3.99-3.97 (m, 1H), 3.69-3.65 (m, 1H), 3.52-3.45 (m, 2H), 2.72-2.68 (m, 1H), 2.13-2.12 (m, 1H), 1.91-1.87 (m, 1H), 1.77-1.70 (m, 2H), 1.52-1.48 (m, 1H), 0.83 (s, 9H); LCMS: 98.7%, m/z [M+H]⁺=598.0; Chiral purity: 98.0%.

### Synthesis of 82.5b

Thionyl chloride (6 mL) was added to 82.4_1b (350 mg, 0.82 mmol) at RT. After stirring for 2 h at RT, the reaction mixture was concentrated under reduced pressure. To the resulting acid chloride in CH₂Cl₂ (3 mL) was added a solution of 3,5-dichloro-N-neopentylaniline (286 mg, 1.23 mmol) in CH₂Cl₂ (2 mL). After stirring for 16 h at 55° C., the reaction mixture was quenched with water (20 mL) and extracted with CH₂Cl₂ (2×20 mL). The combined organic layer was washed with brine, dried over anhydrous Na₂SO₄, filtered and concentrated under reduced pressure. The residue was purified by column chromatography (40 g Silica gel cartridge, 20% EtOAc/pet ether) to afford 82.5b (155 mg, 30%) as a solid.

¹H NMR (400 MHz, CDCl₃): 8.33 (d, J=2.0 Hz, 1H), 7.38 (br s, 1H), 7.33 (br s, 1H), 7.26-7.18 (m, 2H), 5.49-5.44 (m, 1H), 5.14-5.06 (m, 2H), 4.36-4.31 (m, 1H), 4.24-4.19 (m, 1H), 4.12 (d, J=8.0 Hz, 1H), 3.91-3.86 (m, 1H), 3.75-3.70 (m, 2H), 3.48-3.44 (m, 1H), 2.85-2.79 (m, 1H), 2.38-2.33 (m, 1H), 2.06-2.01 (m, 1H), 1.96-1.91 (m, 1H), 1.88-1.81 (m, 1H), 1.72-1.65 (m, 1H), 0.91 (m, 9H); LCMS: 98.1%, m/z [M+H]⁺=638.0.

### Synthesis of 82.6b

To a stirred solution of 82.5b (155 mg, 0.24 mmol) in THF (4 mL) were added aniline (22 mg, 0.24 mmol) and Pd(PPh₃)₄ (56 mg, 0.05 mmol) at RT. After stirring for 2 h at RT, the reaction mixture was concentrated under reduced pressure. The residue was purified by prep. HPLC [KROMOSIL-C18 (150×25) mm, 10 μ; A: 0.1% Formic Acid in H₂O, B: Acetonitrile; Gradient: (Time in minutes/%B): 0/50, 8/90, 10/90, 10.1/98, 12/98, 12.1/50, 14/50 at 22 mL/min) to afford 82.6b (88 mg, 61%) as a solid. ¹H NMR (500 MHz, DMSO-d₆) (Exist in rotameric form): 12.50 (br s, 1H), 11.00/10.99 (s, 1H), 8.37/7.87 (d, J=2.0 Hz, 1H), 7.63-7.62 (m, 1H), 7.53-7.40 (m, 3H), 4.13 (d, J=8.0 Hz, 1H), 4.01-3.98 (m, 1H), 3.76-3.60 (m, 2H), 3.47-3.45 (m, 1H), 2.80-2.69 (m, 1H), 2.27-2.20 (m, 1H), 1.93-1.90 (m, 1H), 1.81-1.75 (m, 2H), 1.57-1.54 (m, 1H), 0.84 (s, 9H); LCMS: 97.4%, m/z [M+H]⁺=598.0; Chiral purity: 96.0%.

TABLE 2

| | | | M/Z | |
|---|---|---|---|---|
| Example | aniline | Compound | (M + H)+ | 1H NMR |
| 83a | 3,5-dichloro-N- (cyclopropylmethyl)aniline | 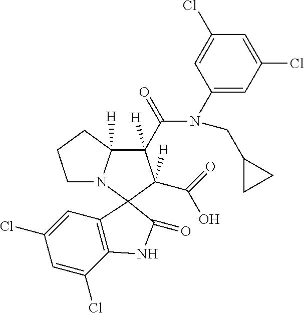 [SMILES: O=C(O)[C@@H]1[C@H](C(=O)N(CC2CC2)c2cc(Cl)cc(Cl)c2)[C@@H]2CCCN2C12C(=O)Nc1c(Cl)cc(Cl)cc12] | 582.0 | 1H NMR (500 MHz, DMSO-d₆) (Exist in rotameric form): 12.35 (br s, 1H), 10.90/10.84 (br s, 1H), 8.39/7.95 (d, J = 2.0 Hz, 1H), 7.70-7.69 (m, 1H), 7.43-7.34 (m, 3H), 4.04 (d, J = 8.0 Hz, 1H), 3.75-3.68 (m, 2H), 3.49-3.44 (m, 1H), 3.36-3.35 (m, 1H), 2.72-2.63 (m, 1H), 2.20-2.10 (m, 1H), 1.89-1.88 (m, 1H), 1.76-1.73 (m, 2H), 1.63-1.52 (m, 1H), 0.88-0.86 (m, 1H), 0.41-0.40 (m, 2H), 0.15-0.13 (m, 1H), 0.07-0.04 (m, 1H). |
| 83b | | 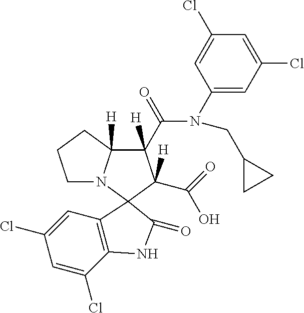 [SMILES: O=C(O)[C@H]1[C@@H](C(=O)N(CC2CC2)c2cc(Cl)cc(Cl)c2)[C@H]2CCCN2C12C(=O)Nc1c(Cl)cc(Cl)cc12] | 582.0 | 1H NMR (500 MHz, DMSO-d₆) (Exist in rotameric form): 12.34 (br s, 1H), 10.90/10.84 (br s, 1H), 8.39/7.95 (d, J = 1.5 Hz, 1H), 7.69-7.67 (m, 1H), 7.43-7.34 (m, 3H), 4.05 (d, J = 8.0 Hz, 1H), 3.75-3.68 (m, 2H), 3.49-3.44 (m, 1H), 3.36-3.32 (m, 1H), 2.73-2.64 (m, 1H), 2.20-2.10 (m, 1H), 1.88-1.87 (m, 1H), 1.76-1.74 (m, 2H), 1.64-1.51 (m, 1H), 0.88-0.86 (m, 1H), 0.41-0.40 (m, 2H), 0.14-0.13 (m, 1H), 0.07-0.04 (m, 1H). |
| 84a | 3,5-dichloro-N- (cyclopentylmethyl)aniline | 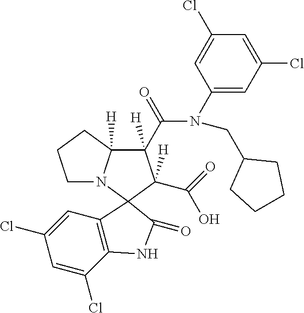 [SMILES: O=C(O)[C@@H]1[C@H](C(=O)N(CC2CCCC2)c2cc(Cl)cc(Cl)c2)[C@@H]2CCCN2C12C(=O)Nc1c(Cl)cc(Cl)cc12] | 610 | 1H NMR (500 MHz, DMSO-d₆) (Exist in rotameric form): 12.36/12.28 (br s, 1H), 10.89/10.84 (br s, 1H), 8.38/7.92 (d, J = 2.0 Hz, 1H), 7.67 (s, 1H), 7.56-7.34 (m, 3H), 4.05 (d, J = 8.0 Hz, 1H), 3.93-3.89 (m, 1H), 3.68-3.66 (m, 1H), 3.54-3.50 (m, 1H), 3.40-3.32 (m, 1H), 2.72-2.61 (m, 1H), 2.18-2.10 (m, 1H), 1.92-1.87 (m, 2H), 1.74-1.71 (m, 2H), 1.67-1.40 (m, 7H), 1.38-1.26 (m, 1H), 1.18-1.10 (m, 1H). |
| 84b | | 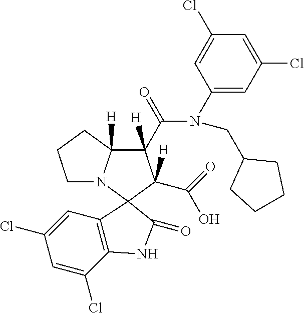 [SMILES: O=C(O)[C@H]1[C@@H](C(=O)N(CC2CCCC2)c2cc(Cl)cc(Cl)c2)[C@H]2CCCN2C12C(=O)Nc1c(Cl)cc(Cl)cc12] | 610 | 1H NMR (500 MHz, DMSO-d₆) (Exist in rotameric form): 12.36/12.29 (br s, 1H), 10.89/10.84 (br s, 1H), 8.38/7.92 (d, J = 1.5 Hz, 1H), 7.67 (s, 1H), 7.56-7.34 (m, 3H), 4.05 (d, J = 7.5 Hz, 1H), 3.92-3.89 (m, 1H), 3.68-3.66 (m, 1H), 3.54-3.50 (m, 1H), 3.37-3.32 (m, 1H), 2.71-2.62 (m, 1H), 2.16-2.09 (m, 1H), 1.92-1.87 (m, 2H), 1.74-1.71 (m, 2H), 1.68-1.40 (m, 7H), 1.38-1.28 (m, 1H), 1.18-1.10 (m, 1H). |

Using the listed anilines, the following compounds were made as in Example 82. Relative stereochemistry was assigned by 2D NMR studies. Absolute stereochemistry unknown for enantiomeric pairs (a and b).

### Example 85a

### Synthesis of (1′S,2′R,7a′S)-5,7-dichloro-1′-((cyclopentylmethyl)(3,5-dichlorophenyl)carbamoyl)-1-methyl-2-oxo-1′,2′,5′,6′,7′,7a′-hexahydrospiro[indoline-3,3′-pyrrolizine]-2′-carboxylic acid

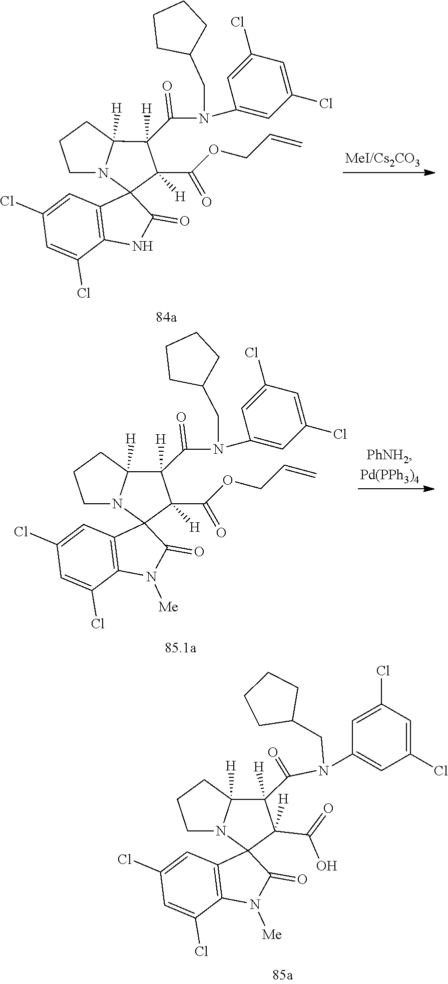 [SMILES: C=CCOC(=O)[C@@H]1[C@H](C(=O)N(CC2CCCC2)c2cc(Cl)cc(Cl)c2)[C@@H]2CCCN2C12C(=O)Nc1c(Cl)cc(Cl)cc12 ; C=CCOC(=O)[C@@H]1[C@H](C(=O)N(CC2CCCC2)c2cc(Cl)cc(Cl)c2)[C@@H]2CCCN2C12C(=O)N(C)c1c(Cl)cc(Cl)cc12 ; CN1C(=O)C2(c3cc(Cl)cc(Cl)c31)[C@H](C(=O)O)[C@H](C(=O)N(CC1CCCC1)c1cc(Cl)cc(Cl)c1)[C@@H]1CCCN12]

### Synthesis of 85.1

To a stirred solution of 84a (110 mg, 0.17 mmol) in CH₃CN (5 mL) was added Cs₂CO₃ (66 mg, 0.20 mmol) followed by Mel (72 mg, 0.50 mmol) and stirred for 6 h at RT. The reaction mixture was quenched with water (10 mL) and extracted with EtOAc (2×20 mL). The combined organic layer was dried over anhydrous Na₂SO₄, filtered and concentrated under reduced pressure to afford 85.1 (110 mg) as solid which was used in the next step without further purification. LCMS: 88.2%, m/z [M+H]⁺=666.0.

### Synthesis of 85

To a stirred solution of 85.1 (110 mg, 0.16 mmol) in THF (3 mL) were added aniline (15 mg, 0.16 mmol) and Pd(PPh₃)₄ (38 mg, 0.03 mmol) at RT. The resulting reaction mixture was stirred for 2 h at RT. After completion of the reaction, the reaction mixture was concentrated under reduced pressure. The residue was purified by prep. HPLC [Column: X-BRIDGE-C18 (150×30), 5 μ; A: 0.1% Formic acid in H₂O, B: Acetonitrile; Gradient: (Time/%B): 0/75, 8/90, 10/95, 12/98, 12.1/75, 14/75 at 25 mL/min] to afford 85 (39 mg, 38%) as a solid. Absolute stereochemistry unknown for enantiomeric pairs (a and b).

¹H NMR (500 MHz, DMSO-d₆) (Exist in rotameric form): 12.40 (br s, 1H), 8.48/8.03 (d, J=2.0 Hz, 1H), 7.68/7.56 (t, J=2.0 Hz, 1H), 7.45/7.41 (d, J=2.5 Hz, 1H), 7.38/7.34 (d, J=1.5 Hz, 2H), 4.08 (d, J=8.0 Hz, 1H), 3.90-3.88 (m, 1H), 3.67-3.65 (m, 1H), 3.55-3.51 (m, 1H), 3.45/3.43 (s, 3H), 3.40-3.37 (m, 1H), 2.68-2.60 (m, 1H), 2.12-2.05 (m, 1H), 1.92-1.87 (m, 2H), 1.80-1.69 (m, 2H), 1.63-1.58 (m, 5H), 1.57-1.46 (m, 2H), 1.38-1.28 (m, 1H), 1.18-1.10 (m, 1H); LCMS: 98.1%, m/z [M+H]⁺=624.0; Chiral purity: 97.4%.

### Example 85b

### Synthesis of (1′R,2′S,7a′R)-5,7-dichloro-1-((cyclopentylmethyl)(3,5-dichlorophenyl)carbamoyl)-1-methyl-2-oxo-1′,2′,5′,6′,7′,7a′-hexahydrospiro[indoline-3,3′-pyrrolizine]-2′-carboxylic acid:

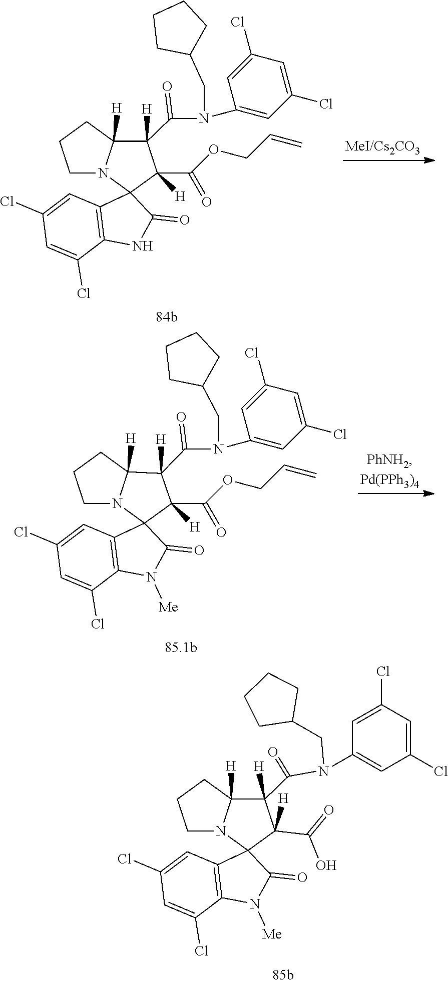 [SMILES: C=CCOC(=O)[C@H]1[C@@H](C(=O)N(CC2CCCC2)c2cc(Cl)cc(Cl)c2)[C@H]2CCCN2C12C(=O)Nc1c(Cl)cc(Cl)cc12 ; C=CCOC(=O)[C@H]1[C@@H](C(=O)N(CC2CCCC2)c2cc(Cl)cc(Cl)c2)[C@H]2CCCN2C12C(=O)N(C)c1c(Cl)cc(Cl)cc12 ; CN1C(=O)C2(c3cc(Cl)cc(Cl)c31)[C@@H](C(=O)O)[C@@H](C(=O)N(CC1CCCC1)c1cc(Cl)cc(Cl)c1)[C@H]1CCCN12]

Compound 85b was prepared from 84b following the procedure described for Example 85a.

¹H NMR (500 MHz, DMSO-d₆) (Exist in rotameric form): 12.39 (br s, 1H), 8.48/8.03 (d, J=2.5 Hz, 1H), 7.68/7.56 (t, J=2.0 Hz, 1H), 7.45/7.41 (d, J=2.0 Hz, 1H), 7.38-7.34 (m, 2H), 4.08 (d, J=8.0 Hz, 1H), 3.92-3.87 (m, 1H), 3.67-3.65 (m, 1H), 3.55-3.51 (m, 1H), 3.45/3.42 (s, 3H), 3.40-3.37 (m, 1H), 2.68-2.60 (m, 1H), 2.10-2.04 (m, 1H), 1.92-1.87 (m, 2H), 1.80-1.69 (m, 2H), 1.63-1.57 (m, 5H), 1.55-1.42 (m, 2H), 1.38-1.27 (m, 1H), 1.17-1.10 (m, 1H); LCMS:

99.6%, m/z [M+H]⁺=624.0; Chiral purity: 97.6%. Absolute stereochemistry unknown for enantiomeric pairs (a and b).

### Example 90 and 91

### Synthesis of (1′R,2′S,3R,6′S,7a′R)-6′-(benzyloxy)-5,7-dichloro-1-((3,5-dichlorophenyl)(neopentyl)carbamoyl)-6′-methyl-2-oxo-1′,2′,5′,6′,7′,7a′-hexahydrospiro[indoline-3,3′-pyrrolizine]-2′-carboxylic acid (90) and (1′R,2′S,3R,6′S,7a′R)-5,7-dichloro-1′-((3,5-dichlorophenyl)(neopentyl)carbamoyl)-6′-hydroxy-6′-methyl-2-oxo-1′,2′,5′,6′,7′,7a′-hexahydrospiro[indoline-3,3′-pyrrolizine]-2′-carboxylic acid (91):

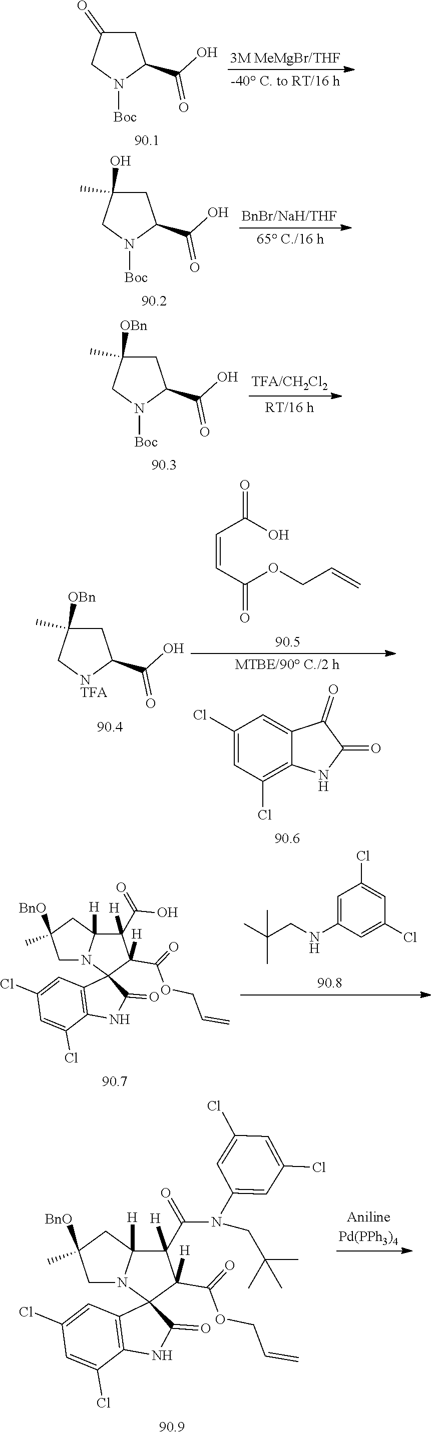

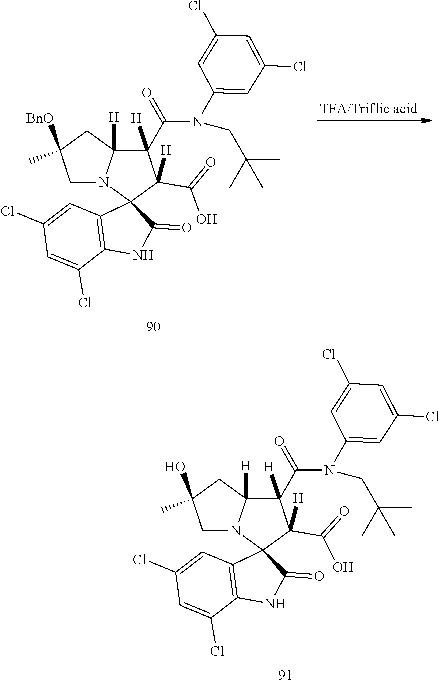 [SMILES: CC(C)(C)CN(C(=O)[C@H]1[C@H]2C[C@](C)(OCc3ccccc3)CN2[C@]2(C(=O)Nc3c(Cl)cc(Cl)cc32)[C@H]1C(=O)O)c1cc(Cl)cc(Cl)c1 ; CC(C)(C)CN(C(=O)[C@H]1[C@H]2C[C@](C)(O)CN2[C@]2(C(=O)Nc3c(Cl)cc(Cl)cc32)[C@H]1C(=O)O)c1cc(Cl)cc(Cl)c1]

### Synthesis of 90.2

To a stirred solution of CH₃MgBr (3M in Et₂O, 728 mL, 2.18 mol) in dry THF (6 L) was added drop wise a solution of 90.1 (200 g, 872 mmol) in dry THF (2 L) through additional funnel over a period of 2 h at −40° C. After slowly warming to RT and stirring for 16 h at RT, the reaction mixture was cooled to −5° C., quenched with 1N HCl until the reaction mixture turned into a clear solution and then extracted with EtOAc (3×3 L). The combined organic layer was washed with H₂O and dried over anhydrous Na₂SO₄, filtered and concentrated. The residue was purified by column chromatography (Silica gel 100-200 mesh, 2% MeOH in DCM) to afford 90.2 (85 g, 40%) as an off white solid.

¹H NMR (500 MHz, DMSO-d₆) (Exist in rotameric form): 12.50-12.30 (br s, 1H), 4.95-4.75 (br s, 1H), 4.14-4.08 (m, 1H), 3.26-3.18 (m, 2H), 2.15-2.08 (m, 1H), 1.95-1.91 (m, 1H), 1.39/1.34 (s, 9H), 1.21 (s, 3H); LCMS: 99.3%, m/z [M−H]⁻=244.1.

### Synthesis of 90.3

To a stirred solution of 90.2 (15 g, 61.1 mmol) in THF (150 ml) was added NaH (14.6 g, 367 mmol) at 0° C. After stiurring at 65° C. for 30 minutes, benzyl bromide (10.9 mL, 91.7 mmol) was added at 65° C. After stirring at 65° C. for 16 h, the reaction mixture was cooled to 0° C., quenched with 10% citric acid solution and extracted with EtOAc (3×100 mL). The combined organic layer was washed with H₂O and dried over anhydrous Na₂SO₄, filtered and concentrated. The residue was purified by column chromatography (Silica gel 100-200 mesh, 30% EtOAc in pet ether) to afford 90.3 (12 g, 58%) as a light brown solid.

¹H NMR (400 MHz, DMSO-d₆): 12.44 (br s, 1H), 7.33-7.22 (m, 5H), 4.42-4.34 (m, 2H), 4.24-4.17 (m, 1H), 3.47 (d, J=11.2 Hz, 1H), 3.32-3.27 (m, 1H), 2.28-2.16 (m, 2H), 1.04-1.36 (m, 12 H); LCMS: 94.8%, m/z [M−H]⁻=334.1.

### Synthesis of 90.4

To a stirred solution of 90.3 (12 g, 35.7 mmol) in CH₂Cl₂ (240 mL) was added TFA (12 mL) at RT. After stirring for 16 h at RT, the reaction mixture was concentrated under reduced pressure to afford 90.4 (12 g) as a brown solid. LCMS: 86.2%, [M-TFA+H]⁺=236.1.

### Synthesis of 90.7

To a stirred solution of 90.4 (3.2 g, 13.6 mmol) in MTBE (100 mL) were added Et₃N (1.9 mL, 13.6 mmol), 90.5 (2.12 g, 13.6 mmol) and 90.6 (2.93 g, 13.6 mmol) and at RT. After stirring at 80° C. for 16 h, the reaction mixture was cooled to RT and concentrated under reduced pressure. The residue was purified by column chromatography (Silica gel 100-200 mesh, 30% EtOAc in pet ether) to afford 90.7 (2.1 g, 30%) as a solid. The regio and relative stereochemistry of 90.7 was confirmed by 2D NMR analysis.

¹H NMR (400 MHz, DMSO-d₆): 12.65 (br s, 1H), 11.00 (s, 1H), 7.68 (s, 1H), 7.46 (s, 1H), 7.27-7.22 (m, 5H), 5.55-5.42 (m, 1H), 5.10-5.04 (m, 2H), 4.38-4.26 (m, 5H), 4.00 (d, J=7.6 Hz, 1H), 3.64-3.60 (m, 1H), 2.67 (d, J=9.6 Hz, 1H), 2.54-2.48 (m, 1H), 2.16-2.09 (m, 1H), 1.61-1.53 (m, 1H), 1.26 (s, 3H); LCMS: 80.2%, m/z [M+H]⁺=545.1.

### Synthesis of 90.9

SOCl₂ (5 mL) was added to 90.7 (1.0 g, 1.83 mmol). After stirring for 2 h at RT, SOCl₂ was evaporated under reduced pressure at 40° C. to afford acid chloride intermediate. To a solution of 90.8 (638 mg, 2.75 mmol) in CH₂Cl₂ (10 mL) was added the acid chloride intermediate at RT. After stirring at 50° C. for 16 h, the reaction mixture was concentrated under reduced pressure. The residue was purified by flash chromatography (40 g Silica gel cartridge, 10% EtOAc in pet ether) to afford 90.9 (950 mg, 70%) as a yellow solid.

¹H NMR (500 MHz, DMSO-d₆): 10.95/10.91 (s, 1H), 8.14/7.69 (d, J=2.0 Hz, 1H), 7.64-7.63 (m, 1H), 7.54-7.42 (m, 3H), 7.33-7.21 (m, 5H), 5.67-5.45 (m, 1H), 5.12-5.10 (m, 2H), 4.37-4.30 (m, 4H), 4.24-4.23 (m, 1H), 4.11 (d, J=8.0 Hz, 1H), 3.95-3.88 (m, 1H), 3.74-3.66 (m, 2H), 2.73 (d, J=9.5 Hz, 1H), 2.50-2.46 (m, 1H), 2.15-2.05 (m, 1H), 1.69-1.59 (m, 1H), 1.28 (s, 3H), 0.84/0.82 (s, 9H); LCMS: 96.0%, m/z [M+H]⁺=760.6.

### Synthesis of 90

To a stirred solution of 90.9 (200 mg, 0.26 mmol) in THF (5 mL) were added aniline (29 mg, 0.32 mmol) and Pd(PPh₃)₄ (61 mg, 0.05 mmol) at RT. After stirring at RT for 2 h, the reaction mixture was diluted with EtOAc (15 mL). The organic solution was collected, washed with brine, dried over anhydrous Na₂SO₄, filtered and concentrated. The residue was purified by flash chromatography (24 g Silica gel cartridge, 20% EtOAc in pet ether) followed by trituration with diethyl ether to afford 90 (45 mg, 24%) as a solid.

¹H NMR (500 MHz, DMSO-d₆) (Exist in rotameric form): 12.49 (br s, 1H), 10.90/10.88 (s, 1H), 8.31/7.85 (d, J=2.0 Hz, 1H), 7.61-7.40 (m, 4H), 7.32-7.20 (m, 5H), 4.39-4.29 (m, 2H), 4.05 (d, J=8.0 Hz, 1H), 3.96-3.89 (m, 2H), 3.61-3.59 (m, 1H), 3.51 (d, J=13.5 Hz, 1H), 2.74 (d, J=8.5 Hz, 1H), 2.43 (d, J=9.0 Hz, 1H), 2.03-2.00 (m, 1H), 1.62-1.58 (m, 1H), 1.26 (s, 3H), 0.87/0.85 (s, 9H); LCMS: 99.0%, m/z [M+H]⁺=718.0; Chiral Purity: 98.8%.

### Synthesis of 91

To a stirred solution 90 (150 mg, 0.21 mmol) in CH₂Cl₂ (3 mL) was added TFA (0.3 mL) and CF₃SO₃H (0.3 mL) at RT. After stirring for 16 h, the reaction mixture was concentrated under reduced pressure. The resulting residue was diluted with EtOAc (25 mL), washed with water (15 mL), brine (15 mL), dried over anhydrous Na₂SO₄, filtered and concentrated under reduced pressure. The residue was purified by prep HPLC [Column: X-SELECT-C₁₈ (150×19), 5 μ; A: 0.1% Formic acid in H₂O, B: Acetonitrile; Gradient: (Time/%B): 0/50, 8/90, 10/90, 10.1/98, 11/98, 11.1/50, 14/50 at 20 mL/minute] to afford 91 (45 mg, 38%) as a solid.

¹H NMR (500 MHz, DMSO-d₆) (Exist in rotameric form): 12.38/12.32 (br s, 1H), 10.90/10.82 (br s, 1H), 8.30/7.79 (s, 1H), 7.62-7.39 (m, 4H), 4.65/4.54 (s, 1H), 4.17-3.80 (m, 3H), 3.62-3.49 (m, 2H), 2.63 (d, J=8.0 Hz, 1H), 2.20/2.10 (d, J=8.0 Hz, 1H), 1.71-1.69 (m, 1H), 1.55-1.51 (m, 1H), 1.15 (s, 3H), 0.83 (s, 9H); LCMS: 99.1%, m/z [M+H]⁺=628.0; Chiral Purity: 99.6%.

TABLE 3

| | | | M/Z | |
|---|---|---|---|---|
| Example | aniline | Compound | (M + H)+ | 1H NMR |
| 92 | 3,5-dichloro-N- methylaniline | 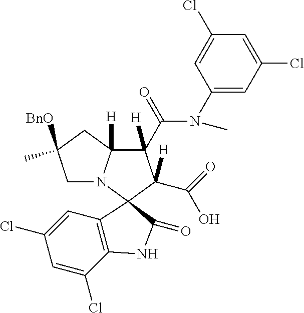 [SMILES: CN(C(=O)[C@H]1[C@H]2C[C@](C)(OCc3ccccc3)CN2[C@]2(C(=O)Nc3c(Cl)cc(Cl)cc32)[C@H]1C(=O)O)c1cc(Cl)cc(Cl)c1] | 662.0 | ¹H NMR (300 MHz, DMSO-d₆) (Exist in rotameric form): 12.38 (br s, 1H), 10.95/10.85 (s, 1H), 8.35/7.87 (d, J = 1.5 Hz, 1H), 7.66/7.53 (t, J = 1.8 Hz, 1H), 7.45-7.42 (m, 3H), 7.31-7.19 (m, 5H), 4.39-4.28 (m, 2H), 4.18-3.90 (m, 2H), 3.57-3.52 (m, 1H), 3.40/3.24 (s, 3H), 2.71 (d, J = 9.3 Hz, 1H), 2.45 (d, J = 9.3 Hz, 1H), 2.07-2.01 (m, 1H), 1.63-1.56 (m, 1H), 1.28/1.24 (s, 3H). |
| 93 | 3,5-dichloro-N- (cyclopropylmethyl)aniline | 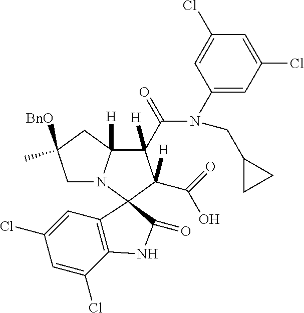 [SMILES: C[C@]1(OCc2ccccc2)C[C@@H]2[C@H](C(=O)N(CC3CC3)c3cc(Cl)cc(Cl)c3)[C@H](C(=O)O)[C@@]3(C(=O)Nc4c(Cl)cc(Cl)cc43)N2C1] | 702 | ¹H NMR (500 MHz, DMSO-d₆) (Exist in rotameric form) 12.37 (br s, 1H), 10.90/10.85 (s, 1H), 8.34/7.93 (d, J = 1.5 Hz, 1H), 7.69/7.59 (t, J = 2.0 Hz, 1H), 7.45-7.20 (m, 8H), 4.37-4.30 (m, 2H), 4.02 (d, J = 8.0 Hz, 1H), 3.98-3.90 (m, 1H), 3.75-3.68 (m, 1H), 3.52-3.48 (m, 1H), 3.45-3.42 (m, 1H), 2.71 (d, J = 9.0 Hz, 1H), 2.44 (d, J = 9.0 Hz, 1H), 2.10-2.02 (m, 1H), 1.68-1.64 (m, 1H), 1.28 (s, 3H), 0.98-0.84 (m, 1H), 0.43-0.41 (m, 2H), 0.17-0.09 (m, 2H). |
| 94 | 3,5-dichloro-N- (cyclopentylmethyl)aniline | 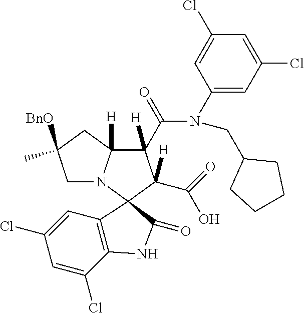 [SMILES: C[C@]1(OCc2ccccc2)C[C@@H]2[C@H](C(=O)N(CC3CCCC3)c3cc(Cl)cc(Cl)c3)[C@H](C(=O)O)[C@@]3(C(=O)Nc4c(Cl)cc(Cl)cc43)N2C1] | 730.0 | ¹H NMR (500 MHz, DMSO-d₆) (Exist in rotameric form): 12.38 (s, 1H), 10.90/10.85 (s, 1H), 8.33/7.90 (d, J = 2.0 Hz, 1H), 7.67/7.56 (m, 1H), 7.45-7.40 (m, 3H), 7.37-7.22 (m, 5H), 4.41-4.30 (m, 2H), 4.30 (d, J = 8.0 Hz, 1H), 3.93-3.89 (m, 2H), 3.56-3.52 (m, 1H), 3.47-3.46 (m, 1H), 2.71 (d, J = 9.0 Hz, 1H), 2.44 (d, J = 9.0 Hz, 1H), 2.07-1.99 (m, 1H), 1.93-1.90 (m, 1H), 1.66-1.58 (m, 5H), 1.49-1.45 (m, 2H), 1.38-1.30 (m, 1H), 1.27 (s, 3H), 1.20-1.11 (m, 1H). |
| 95 | 3,5-dichloro-N-(2,2,3,3- tetramethylbutyl)aniline | 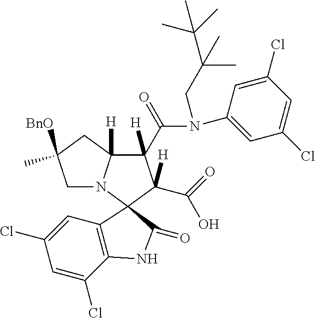 [SMILES: CC(C)(C)C(C)(C)CN(C(=O)[C@H]1[C@H]2C[C@](C)(OCc3ccccc3)CN2[C@]2(C(=O)Nc3c(Cl)cc(Cl)cc32)[C@H]1C(=O)O)c1cc(Cl)cc(Cl)c1] | 760.2 | ¹H NMR (400 MHz, DMSO-d₆) (Exist in rotameric form): 12.37 (br s, 1H), 10.90/10.85 (s, 1H), 8.27/7.78 (s, 1H), 7.61-7.40 (m, 4H), 7.33-7.20 (m, 5H), 4.36-4.29 (m, 2H), 4.10 (d, J = 14.0 Hz, 1H), 3.99 (d, J = 7.6 Hz, 1H), 3.91-3.85 (m, 1H), 3.73-3.63 (m, 2H), 2.74 (d, J = 9.2 Hz, 1H), 2.44 (d, J = 9.2 Hz, 1H), 2.04-1.99 (m, 1H), 1.67-1.61 (m, 1H), 1.27 (s, 3H), 0.89 (s, 9H), 0.85 (s, 3H), 0.67 (s, 3H). |
| 96 | 3,5-dichloro-N-(2,2,3,3- tetramethylbutyl)aniline | 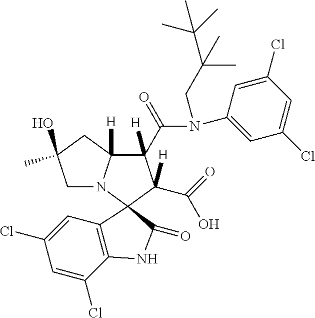 [SMILES: CC(C)(C)C(C)(C)CN(C(=O)[C@H]1[C@H]2C[C@](C)(O)CN2[C@]2(C(=O)Nc3c(Cl)cc(Cl)cc32)[C@H]1C(=O)O)c1cc(Cl)cc(Cl)c1] | 670.1 | ¹H NMR (400 MHz, DMSO-d₆) (Exist in rotameric form): 12.35 (br s, 1H), 10.90/10.82 (s, 1H), 8.29/7.73 (d, J = 2.0 Hz, 1H), 7.62 (t, J = 1.6 Hz, 1H), 7.52-7.49 (m, 2H), 7.43/7.39 (d, J = 2.0 Hz, 1H), 4.56 (s, 1H), 4.08-4.00 (m, 2H), 3.93-3.87 (m, 1H), 3.71 (d, J = 13.6 Hz, 1H), 3.59 (t, J = 7.6 Hz, 1H), 2.61 (d, J = 8.4 Hz, 1H), 2.12 (d, J = 8.4 Hz, 1H), 1.68-1.64 (m, 1H), 1.57-1.52 (m, 1H), 1.14/1.10 (s, 3H), 0.91/0.88 (s, 9H), 0.81 (s, 3H), 0.65 (s, 3H). |

Using the listed anilines, the following compounds were made as in Example 90 or 91 with intermediate 90.7 and listed aniline.

### Example 97

### Synthesis of (1′R,2′S,3R,6′S,7a′R)-6′-(benzyloxy)-5,7-dichloro-1′-((cyclopentylmethyl)(3,5-dichlorophenyl)carbamoyl)-1,6′-dimethyl-2-oxo-1′,2′,5′,6′,7′,7a′-hexahydrospiro[indoline-3,3′-pyrrolizine]-2′-carboxylic acid:

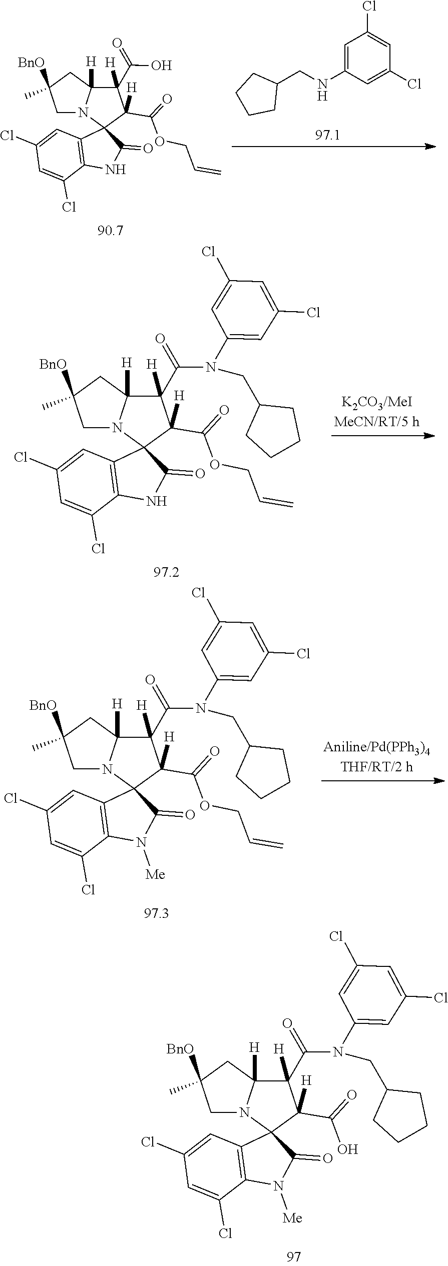 [SMILES: C=CCOC(=O)[C@H]1[C@@H](C(=O)O)[C@H]2C[C@](C)(OCc3ccccc3)CN2[C@]12C(=O)Nc1c(Cl)cc(Cl)cc12 ; Clc1cc(Cl)cc(NCC2CCCC2)c1 ; C=CCOC(=O)[C@H]1[C@@H](C(=O)N(CC2CCCC2)c2cc(Cl)cc(Cl)c2)[C@H]2C[C@](C)(OCc3ccccc3)CN2[C@]12C(=O)Nc1c(Cl)cc(Cl)cc12 ; C=CCOC(=O)[C@H]1[C@@H](C(=O)N(CC2CCCC2)c2cc(Cl)cc(Cl)c2)[C@H]2C[C@](C)(OCc3ccccc3)CN2[C@]12C(=O)N(C)c1c(Cl)cc(Cl)cc12 ; CN1C(=O)[C@]2(c3cc(Cl)cc(Cl)c31)[C@@H](C(=O)O)[C@@H](C(=O)N(CC1CCCC1)c1cc(Cl)cc(Cl)c1)[C@H]1C[C@](C)(OCc3ccccc3)CN12]

### Synthesis of 97.2

SOCl₂ (10 mL) was added to 90.7 (1.2 g, 2.20 mmol). After stirring for 2 h at RT, SOCl₂ was evaporated under reduced pressure to afford acid chloride intermeidate. To a solution of 97.1 (805 mg, 3.30 mmol) in CH₂Cl₂ (15 mL) was added to the above prepared acid chloride intermediate at RT. After stirring at RT for 16 h, the reaction mixture was concentrated under reduced pressure. The resulting residue was purified by flash column chromatography (40 g Silica gel cartridge, 15% EtOAc in pet ether) to afford 97.2 (800 mg, 50%) as a solid.

¹H NMR (400 MHz, CDCl₃): 8.35 (d, J=1.6 Hz, 1H), 7.42 (s, 1H), 7.36 (t, J=1.6 Hz, 1H), 7.33-7.17 (m, 7H), 5.51-5.44 (m, 1H), 5.14-5.06 (m, 2H), 4.40-4.32 (m, 3H), 4.27-4.17 (m, 2H), 4.11 (d, J=8.0 Hz, 1H), 3.89-3.84 (m, 1H), 3.67-3.61 (m, 1H), 3.36 (t, J=8.0 Hz, 1H), 2.86 (d, J=9.2 Hz, 1H), 2.64 (d, J=8.8 Hz, 1H), 2.10-2.00 (m, 2H), 1.80-1.66 (m, 5H), 1.39 (s, 3H), 1.29-1.24 (m, 3H), 0.93-0.83 (m, 1H); LCMS: 97.3%, m/z [M+H]⁺=772.49.

### Synthesis of 97.3

To a stirred solution of 97.2 (300 mg, 0.39 mmol) in CH₃CN (5 mL) were added K₂CO₃ (80 mg, 0.58 mmol) and CH₃I (0.05 mL, 0.78 mmol) at RT. After stirring at RT for 5 h, the reaction mixture was diluted with EtOAc (15 mL). The organic solution was washed with water (20 mL), brine (20 mL), dried over anhydrous Na₂SO₄, filtered and concentrated under reduced pressure. The residue was purified by flash column chromatography (24 g Silica gel cartridge, 5% EtOAc in pet ether) to afford 97.3 (220 mg, 72%) as a solid.

¹H NMR (400 MHz, CDCl₃): 8.37 (d, J=2.0 Hz, 1H), 7.37-7.16 (m, 9H), 5.52-5.48 (m, 1H), 5.17-5.07 (m, 2H), 4.41-4.15 (m, 5H), 4.06 (d, J=8.0 Hz, 1H), 3.89-3.84 (m, 1H), 3.65-3.60 (m, 1H), 3.51 (s, 3H), 3.39-3.35 (m, 1H), 2.78 (d, J=8.8 Hz, 1H), 2.56 (d, J=8.8 4 Hz, 1H), 2.06-2.02 (m, 2H), 1.76-1.53 (m, 6H), 1.39 (s, 3H), 1.28-1.24 (m, 3H); LCMS: 94.4%, m/z [M+H]⁺=786.4.

### Synthesis of 97

To a stirred solution of 97.3 (220 mg, 0.28 mmol) in THF (3 mL) were added aniline (26 mg, 0.28 mmol) and Pd(PPh₃)₄ (64 mg, 0.06 mmol) at RT. After stirring for 2 h, the reaction mixture was concentrated under pressure. The residue was purified by prep. HPLC [Column: KROMOSIL-C18 (150×25) mm, 10 μ, A: 0.1% Formic acid in H₂O, B: Acetontrile; Gradient: (Time/%B): 0/80, 8/95, 11/95, 11/98, 12.1/98, 12.1/80, 15/80 at 25 mL/minute] to afford 97 (58 mg, 27%) as a solid.

¹H NMR (500 MHz, DMSO-d₆) (exist in rotameric form): 12.43 (s, 1H), 8.42/8.00 (d, J=2.5 Hz, 1H), 7.67/7.56 (t, J=2.0 Hz, 1H), 7.47 (d, J=2.0 Hz, 1H), 7.43-7.41 (m, 2H), 7.37-7.23 (m, 5H), 4.36-4.29 (m, 2H), 4.04 (d, J=8.0 Hz, 1H), 3.92-3.88 (m, 2H), 3.57-3.53 (m, 1H), 3.50-3.45 (m, 1H), 3.45/3.41 (s, 3H), 2.67 (d, J=9.0 Hz, 1H), 2.37 (d, J=9.0 Hz, 1H), 2.05-1.95 (m, 1H), 1.95-1.87 (m, 1H), 1.65-1.58 (m, 5H), 1.48-1.44 (m, 2H), 1.35-1.28 (m, 1H), 1.28 (s, 3H), 1.19-1.12 (m, 1H); LCMS: 99.1%, m/z [M+H]⁺=743.9; Chiral Purity: 99.8%.

### Example 100

### Synthesis of rac-(1′R,2′S,7a′R)-6,7-dichloro-1-((3,5-dichlorophenyl)(methyl)carbamoyl)-6′,6′-difluoro-2-oxo-1′,2′,5′,6′,7′,7a′-hexahydrospiro[indoline-3,3′-pyrrolizine]-2′-carboxylic acid (100.6b)

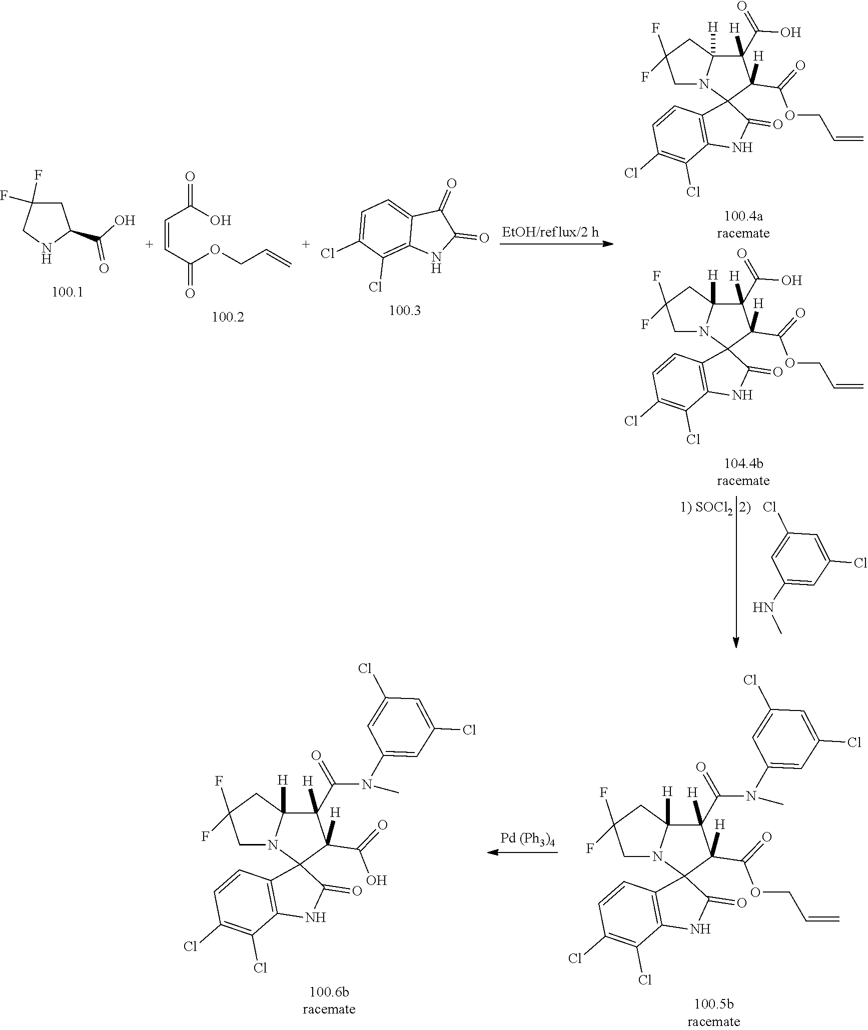

### Synthesis of 100.4a & 100.4b

To a solution of 100.2 (500 mg, 3.18 mmol) in EtOH (10 mL) was added 100.1 (789 mg, 3.18 mmol) and 100.3 (680 mg, 3.18 mmol) at RT. After refluxing for 2 h, the reaction mixture was concentrated under reduced pressure. The residue was purified by flash chromatography (80 g Silica gel cartridge, 30%-35% EtOAc/pet ether) to afford minor diastereomer 100.4a (150 mg, 10%) as a white solid and major diastereomer 100.4b (250 mg, 17%) as a white solid.

100.4a: ¹H NMR (500 MHz, DMSO-d₆): 12.65 (s, 1H), 11.13 (s, 1H), 7.54 (d, J=8.0 Hz, 1H), 7.28 (d, J=8.0 Hz, 1H), 5.86-5.80 (m, 1H), 5.27-5.15 (m, 2H), 4.47-4.39 (m, 3H), 3.73-3.68 (m, 2H), 3.10-2.95 (m, 1H), 2.88-2.76 (m, 1H), 2.62-2.40 (m, 2H); LCMS: 88.6%, m/z [M+H]⁺=461.0. Regiochemistry and relative stereochemistry was confirmed by 2D NMR studies.

100.4b: ¹H NMR (400 MHz, DMSO-d₆): 12.80 (s, 1H), 11.17 (s, 1H), 7.55 (d, J=8.4 Hz, 1H), 7.25 (d, J=8.4 Hz, 1H), 5.53-5.44 (m, 1H), 5.11-5.04 (m, 2H), 4.34-4.22 (m, 2H), 4.15-4.09 (m, 1H), 3.92 (d, J=7.6 Hz, 1H), 3.76-3.72 (m, 1H), 3.32-3.24 (m, 1H), 2.75-2.67 (m, 1H), 2.51-2.40 (m, 1H), 2.35-2.15 (m, 1H); LCMS: 89.1%, m/z [M−H]⁻=459.0. Regiochemistry and relative stereochemistry was confirmed by 2D NMR studies.

### Synthesis of 100.5b

Thionyl chloride (3 mL) was added to 100.4b (250 mg, 0.54 mmol) at RT. After stirring for 2 h, the reaction mixture was concentrated under reduced pressure to afford residue of acid chloride. To the acid chloride in CH₂Cl₂ (5 mL) was added a solution of 3,5-dichloro-N-methylaniline (144 mg, 0.81 mmol) in CH₂Cl₂ (5 mL). After stirring for 16 h at RT, the reaction was quenched with water (10 mL). The organic layer was separated, and the aqueous layer was extracted with CH₂Cl₂ (2×15 mL). The combined organic layer was washed with brine, dried over anhydrous Na₂SO₄, filtered and concentrated under reduced pressure. The residue was purified by prep. HPLC (X-BRIDGE-C18 (150×30) mm, 5u; A: 0.1% Formic Acid in H₂O, B: Acetonitrile; Gradient: (T%B):- 0/70, 8/85, 11/85, 11.1/98, 12/98, 12.1/70 ,15/70 at 20 mL/min) to afford 100.5b (80 mg, 24%) as a solid. LCMS: 96.6%, m/z [M+H]⁺=618.1.

### Synthesis of 100.6b

To a stirred solution of 100.5b (80 mg, 0.13 mmol) in THF (2 mL) were added aniline (10 mg, 0.13 mmol) and Pd(PPh₃)₄ (29 mg, 0.03 mmol) at RT. After stirring for 2 h at RT, the reaction mixture was concentrated under reduced pressure. The resulting residue was purified by prep. HPLC [ATLANTIS-T3 (250×20) mm, 5 μ; A: 10 mM Ammonium bicarbonate in H₂O, B: Acetonitrile; Gradient: (Time/%B): 0/55, 8/80, 11/90, 11.1/98, 13/98, 13.1/55, 16/55 at 18 mL/min] to afford 100.6b (25 mg, 34%) as a solid.

¹H NMR (500 MHz, DMSO-d₆) (Exist in rotameric form): 12.44 (br s, 1H), 11.10/11.01 (s, 1H), 8.20/7.80 (d, J=8.0 Hz, 1H), 7.66-7.20 (m, 4H), 4.25-4.07 (m, 1H), 3.99 (d, J=7.5 Hz, 1H), 3.85-3.82 (m, 1H), 3.55 (t, J=7.5 Hz, 1H), 3.39/3.23 (s, 3H), 2.60-2.50 (m, 1H), 2.42-2.35 (m, 1H), 2.25-2.05 (m, 1H); LCMS: 95.2%, m/z [M+H]⁺=578.0; Chiral purity: (49.1+48.6)%.

TABLE 4

| Example | Isatin | Compound | M/Z | 1H NMR |
|---|---|---|---|---|
| 101 | 6-Chloroisatin | 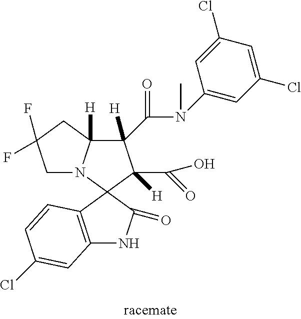 [SMILES: CN(C(=O)[C@H]1[C@H]2CC(F)(F)CN2C2(C(=O)Nc3cc(Cl)ccc32)[C@H]1C(=O)O)c1cc(Cl)cc(Cl)c1] | 542 [M − H]⁻ | 1H NMR (500 MHz, DMSO-d₆) (Exist in rotameric form): 12.36 (br s, 1H), 10.59/10.51 (s, 1H), 8.17/7.79 (d, J = 8.0 Hz, 1H), 7.65-7.40 (m, 3H), 7.16-6.96 (m, 1H), 6.80 (d, J = 1.5 Hz, 1H), 4.19/4.03 (m, 1H), 3.91 (d, J = 7.5 Hz, 1H), 3.83-3.79 (m, 1H), 3.55 (t, J = 7.5 Hz, 1H), 3.39/3.23 (s, 3H), 2.60-2.50 (m, 1H), 2.41-2.33 (m, 1H), 2.23-2.07 (m, 1H). |
| 102 | Isatin | 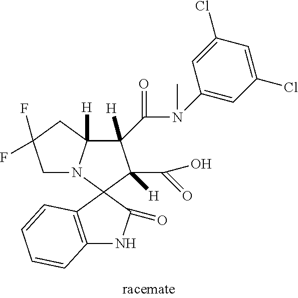 [SMILES: CN(C(=O)[C@H]1[C@H]2CC(F)(F)CN2C2(C(=O)Nc3ccccc32)[C@H]1C(=O)O)c1cc(Cl)cc(Cl)c1] | 510.1 [M + H]⁺ | ¹H NMR (500 MHz, DMSO-d₆) (Exist in rotameric form): 12.27 (br s, 1H), 10.40/10.34 (s, 1H), 8.07/7.74 (d, J = 7.5 Hz, 1H), 7.65-7.42 (m, 3H), 7.23-7.20 (m, 1H), 7.01-6.79 (m, 2H), 4.28-4.20/4.00-3.96 (m, 1H), 3.82-3.64 (m, 2H), 3.55-3.48 (m, 1H), 3.39/3.23 (s, 3H), 2.50-2.44 (m, 1H), 2.40-2.20 (m, 2H). |
| 103 | 5- Methoxyisatin | 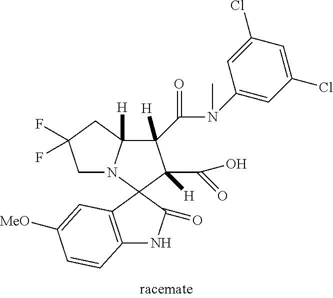 [SMILES: COc1ccc2c(c1)C1(C(=O)N2)[C@@H](C(=O)O)[C@@H](C(=O)N(C)c2cc(Cl)cc(Cl)c2)[C@H]2CC(F)(F)CN21] | 540.1 [M + H]⁺ | ¹H NMR (500 MHz, DMSO-d₆) (Exist in rotameric form): 12.30 (br s, 1H), 10.25/10.17 (br s, 1H), 7.83-7.44 (m, 4H), 6.89-6.46 (m, 2H), 4.25-4.00 (m, 1H), 3.85-3.59 (m, 2H), 3.69/3.64 (s, 3H), 3.52-3.44 (m, 1H), 3.39/3.23 (s, 3H), 2.60-2.50 (m, 1H), 2.40-2.15 (m, 2H). |
| 104 | 7- chloroindoline- 2,3-dione | 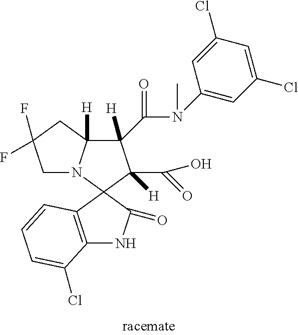 [SMILES: CN(C(=O)[C@H]1[C@H]2CC(F)(F)CN2C2(C(=O)Nc3c(Cl)cccc32)[C@H]1C(=O)O)c1cc(Cl)cc(Cl)c1] | 544.1 [M + H]⁺ | ¹H NMR (500 MHz, DMSO-d₆) (Exist in rotameric form): 12.37 (br s, 1H), 10.86/10.78 (s, 1H), 8.15/7.77 (d, J = 7.5 Hz, 1H), 7.66 (t, J = 1.5 Hz, 1H), 7.55-7.39 (m, 2H), 7.31-7.26 (m, 1H), 7.04-6.91 (m, 1H), 4.21-4.06 (m, 1H), 3.94 (d, J = 8.0 Hz, 1H), 3.85-3.81 (m, 1H), 3.58 (t, J = 7.0 Hz, 1H), 3.45-3.37 (m, 1H), 3.39/3.23 (s, 3H), 2.40-2.33 (m, 1H), 2.24-2.17 (m, 1H). |

Using various isatins in place of 6,7-dichloroindoline-2,3-dione, 100.3, as in Example 100, the following compounds were made.

### Example 110

### Synthesis of (1′R,2′S,3R,7a′R)-5,7-dichloro-1-((3,5-dichlorophenyl)(neopentyl)carbamoyl)-6′,6′-difluoro-2-oxo-1′,2′,5′,6′,7′,7a′-hexahydrospiro[indoline-3,3′-pyrrolizine]-2′-carboxylic acid (110)

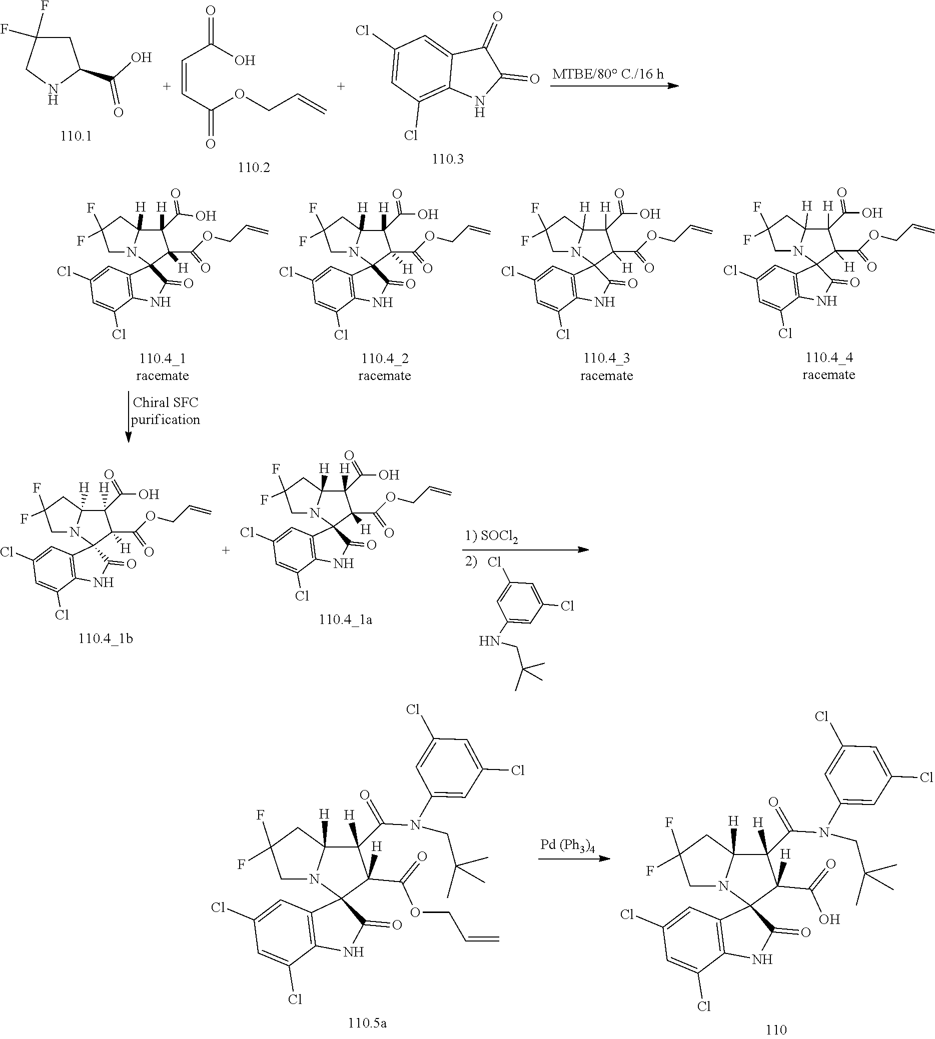

### Synthesis of 110.4_1 & 110.4_2 & 110.4_3 & 110.4_4

To a solution of (S)-4,4-difluoropyrrolidine-2-carboxylic acid, 110.1, (10 g, 66.2 mmol) in MTBE (200 mL) were added (Z)-4-(allyloxy)-4-oxobut-2-enoic acid, 110.2, (10.3 g, 66.2 mmol) and 5,7-dichloroindoline-2,3-dione, 110.3, (14.3 g, 66.2 mmol) at RT. After stirring at reflux for 16 h, the reaction mixture was concentrated under reduced pressure. The residue was purified by column chromatography (silica-gel, 100-200 mesh, gradient 20%-25% EtOAc/pet ether) to give 110.4_1, 110.4_2, 110.4_3 & 110.4_4. (LCMS ratio: 25:15:6:5).

110.4_1: rac-(1′R,2′S,3R,7a′R)-2′-((allyloxy)carbonyl)-5,7-dichloro-6′,6′-difluoro-2-oxo-1′,2′,5′,6′,7′,7a′-hexahydrospiro[indoline-3,3′-pyrrolizine]-1′-carboxylic acid ¹H NMR (500 MHz, DMSO-d₆): 12.89 (br s, 1H), 11.15 (s, 1H), 7.69 (d, J=2.0 Hz, 1H), 7.50 (d, J=2.0 Hz, 1H), 5.47-5.44 (m, 1H), 5.11-5.06 (m, 2H), 4.28-4.27 (m, 2H), 4.07-4.02 (m, 2H), 3.64 (t, J=7.0 Hz, 1H), 3.23-3.15 (m, 1H), 2.75-2.65 (m, 1H), 2.51-2.49 (m, 1H), 2.19-2.08 (m, 1H), ¹⁹F NMR (376.49 MHz, DMSO-d₆): −89.57 (d, J=226 Hz), −94.10 (d, J=226 Hz); LCMS 98.0%, m/z [M+H]⁺=461.0. Relative regiochemistry was confirmed by 2D NMR studies.

110.4_2: rac-(1′R,2′R,3R,7a′R)-2′-((allyloxy)carbonyl)-5,7-dichloro-6′,6′-difluoro-2-oxo-1′,2′,5′,6′,7′,7a′-hexahydrospiro[indoline-3,3′-pyrrolizine]-1′-carboxylic acid ¹H NMR (400 MHz, DMSO-d₆): 8.96 (br s, 1H), 7.62 (d, J=2.0 Hz, 1H), 7.54 (d, J=2.0 Hz, 1H), 5.85-5.79 (m, 1H), 5.30-5.13 (m, 2H), 4.48-4.35 (m, 3H), 3.56-3.53 (m, 2H), 3.00-2.79 (m, 2H), 2.51-2.40 (m, 2H); ¹⁹F NMR (376.49 MHz, DMSO-d₆): −92.55 (d, J=228 Hz), −100.21 (d, J=228 Hz); LCMS 97.8%, m/z [M+H]⁺=461.0. Relative regiochemistry was confirmed by 2D NMR studies.

110.4_3: ¹H NMR (400 MHz, DMSO-d₆): 12.75 (br s, 1H), 11.09 (s, 1H), 7.77 (d, J=2.0 Hz, 1H), 7.48 (d, J=2.0 Hz, 1H), 6.01-5.91 (m, 1H), 5.42-5.37 (m, 1H), 5.26 (dd, J=10.4 Hz, 1.6 Hz, 1H), 4.71-4.61 (m, 2H), 4.06-3.94 (m, 2H), 3.62 (t, J=6.4 Hz, 1H), 3.18-3.09 (m, 1H), 2.70-2.61 (m, 1H), 2.49-2.32 (m, 1H), 2.09-1.98 (m, 1H); ¹⁹F NMR (376.49 MHz, DMSO-d₆): −89.13 (d, J=231 Hz), −92.96 (d, J=231 Hz); LCMS 99.6%, m/z [M+H]⁺=460.9. Unknown relative regiochemistry.

110.4_4: ¹H NMR (400 MHz, DMSO-d₆): 12.87 (br s, 1H), 11.42 (s, 1H), 7.54 (d, J=2.0 Hz, 1H), 6.87 (d, J=2.0 Hz, 1H), 5.76-5.66 (m, 1H), 5.23-5.16 (m, 2H), 4.51-4.35 (m, 3H), 3.87 (t, J=8.0 Hz, 1H), 3.58 (d, J=7.2 Hz, 1H), 3.08-3.01 (m, 1H), 2.89-2.67 (m, 1H), 2.58-2.32 (m, 2H); ¹⁹F NMR (376.49 MHz, DMSO-d₆): −92.33 (d, J=230 Hz), −101.91 (d, J=230 Hz); LCMS 95.2%, m/z [M+H]⁺=461.0. Unknown relative regiochemistry.

### Separation of 110.4_1a & 110.4_1b

110.4_1 (45 g) was purified by chiral SFC using Chiral pack IG (250×30) mm, 5 μ; 0.2% TFA in n-hexane: Isopropanol (85:15) at rt (isocratic 42.0 mL/min, 13 min run time with detection at 254 nm). Pure fractions were concentrated under reduced pressure to give 20 g of 110.4_1a (Peak-1) and 14.7 g of 110.4_1b (Peak-2) as white solids.

110.4_1a: (1′R,2′S,3R,7a′R)-2′-((allyloxy)carbonyl)-5,7-dichloro-6′,6′-difluoro-2-oxo-1′,2′,5′,6′,7′,7a′-hexahydrospiro[indoline-3,3′-pyrrolizine]-1′-carboxylic acid. [α]²⁵D+78.8 (c 1.0, MeOH); ¹H NMR (500 MHz, DMSO-d₆): 12.86 (s, 1H), 11.16 (s, 1H), 7.68 (d, J=2.0 Hz, 1H), 7.51 (d, J=2.0 Hz, 1H), 5.49-5.43 (m, 1H), 5.11-5.06 (m, 2H), 4.28-4.27 (m, 2H), 4.07-4.02 (m, 2H), 3.64 (t, J=6.5 Hz, 1H), 3.21-3.18 (m, 1H), 2.69-2.52 (m, 1H), 2.51-2.50 (m, 1H), 2.15-2.05 (m, 1H); ¹⁹F NMR (376.49 MHz, DMSO-d₆): −89.56 (d, J=226 Hz), −94.08 (d, J=226 Hz); LCMS 98.9%, m/z [M+H]⁺=461.2; Chiral purity: 99.8%. Absolute stereochemistry was determined by single crystal x-ray diffraction analysis.

110.4_1b: (1′S,2′R,3S,7a′S)-2′-((allyloxy)carbonyl)-5,7-dichloro-6′,6′-difluoro-2-oxo-1′,2′,5′,6′,7′,7a′-hexahydrospiro[indoline-3,3′-pyrrolizine]-1′-carboxylic acid [α]²⁵D−73.2 (c 1.0, MeOH); ¹H NMR (400 MHz, DMSO-d₆): 12.88 (br s, 1H), 11.16 (s, 1H), 7.69 (d, J=2.0 Hz, 1H), 7.50 (d, J=2.0 Hz, 1H), 5.50-5.42 (m, 1H), 5.11-5.06 (m, 2H), 4.28-4.26 (m, 2H), 4.09-4.01 (m, 2H), 3.64 (t, J=7.2 Hz, 1H), 3.23-3.14 (m, 1H), 2.74-2.65 (m, 1H), 2.53-2.44 (m, 1H), 2.16-2.08 (m, 1H); ¹⁹F NMR (376.49 MHz, DMSO-d₆): −89.56 (d, J=226 Hz), −94.08 (d, J=226 Hz); LCMS 98.7%, m/z [M+H]⁺=461.0; Chiral purity: 99.9%.

### Synthesis of 110.5a

Thionyl chloride (8 mL) was added to 110.4_1a (500 mg, 1.08 mmol) at RT. After stirring for 2 h at RT, the reaction mixture was concentrated under reduced pressure. To the resulting residue dissolved in CH₂Cl₂(10 mL) was a solution of 3,5-dichloro-N-neopentylaniline (500 mg, 2.16 mmol) in CH₂Cl₂ (5 mL). After stirring for 16 h at 55° C., the reaction mixture was quenched with water (20 mL) and extracted with CH₂Cl₂ (2×20 mL). The combined organic layer was washed with brine, dried over anhydrous Na₂SO₄, filtered and concentrated under reduced pressure. The residue was purified by column chromatography (40 g silica gel cartridge, gradient 10% EtOAc/pet ether) to afford 110.5a (410 mg, 53%) as a solid.

¹H NMR (400 MHz, CDCl₃): 8.28 (d, J=2.0 Hz, 1H), 7.43 (s, 1H), 7.34 (t, J=1.6 Hz, 1H), 7.27-7.26 (m, 2H), 5.48-5.42 (m, 1H), 5.14-5.06 (m, 2H), 4.36-4.31 (m, 1H), 4.24-4.19 (m, 1H), 4.04-3.97 (m, 2H), 3.81-3.68 (m, 2H), 3.51 (t, J=7.6 Hz, 1H), 3.33-3.26 (m, 1H), 2.77-2.69 (m, 1H), 2.31-2.17 (m, 2H), 0.91 (s, 9H); LCMS 99.3%, m/z [M+H]⁺=674.0.

### Synthesis of 110

To a stirred solution of 110.5a (400 mg, 0.59 mmol) in THF (10 mL) were added aniline (55 mg, 0.59 mmol) and Pd(PPh₃)₄ (136 mg, 0.11 mmol) at RT. After stirring for 2 h at RT, the reaction mixture was concentrated under reduced pressure. The residue was purified by prep-HPLC (KROMOSIL-C18 (150×30) mm, 10 μ; A: 0.1% Formic acid in H₂O; B: acetonitrile; gradient: (Time in minutes/%B): 0/70, 8/90, 10/95 at 24 mL/min) to afford 110 (161 mg, 42%) as a solid.

[α]²⁵D −6.41 (c 0.5, MeOH); ¹H NMR (500 MHz, DMSO-d₆): 12.65 (br s, 1H), 10.98 (s, 1H), 8.26 (d, J=2.0 Hz, 1H), 7.81-7.43 (m, 4H), 4.05 (d, J=7.5 Hz, 1H), 4.01 (d, J=14 Hz, 1H), 3.84-3.69 (m, 1H), 3.60 (dd, J=7.5 Hz, J=6.5 Hz, 1H), 3.44 (d, J=14.0 Hz, 1H), 3.32-3.24 (m, 1H), 2.63-2.52 (m, 1H), 2.40-2.32 (m, 1H), 2.11-2.04 (m, 1H), 0.84/0.82 (s, 9H); ¹⁹F NMR (470.59 MHz, DMSO-d₆): −89.21 (d, J=226 Hz), −97.17 (d, J=226 Hz); LCMS 99.3%, m/z [M+H]⁺=633.9; Chiral purity: 98.9%.

### Example 111

### Synthesis of (1′S,2′R,3S,7a′S)-5,7-dichloro-1-((3,5-dichlorophenyl)(neopentyl)carbamoyl)-6′,6′-difluoro-2-oxo-1′,2′,5′,6′,7′,7a′-hexahydrospiro[indoline-3,3′-pyrrolizine]-2′-carboxylic acid

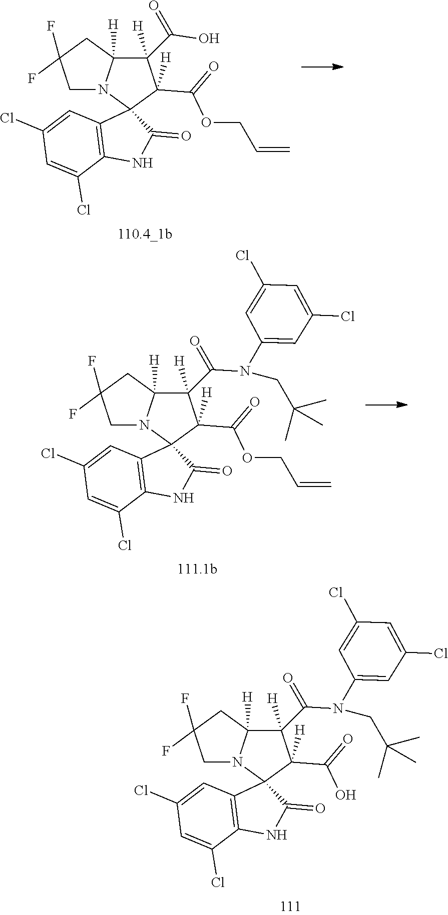 [SMILES: C=CCOC(=O)[C@@H]1[C@H](C(=O)O)[C@@H]2CC(F)(F)CN2[C@@]12C(=O)Nc1c(Cl)cc(Cl)cc12 ; C=CCOC(=O)[C@@H]1[C@H](C(=O)N(CC(C)(C)C)c2cc(Cl)cc(Cl)c2)[C@@H]2CC(F)(F)CN2[C@@]12C(=O)Nc1c(Cl)cc(Cl)cc12 ; CC(C)(C)CN(C(=O)[C@H]1[C@@H](C(=O)O)[C@]2(C(=O)Nc3c(Cl)cc(Cl)cc32)N2CC(F)(F)C[C@@H]12)c1cc(Cl)cc(Cl)c1]

### Synthesis of 111.1b

Thionyl chloride (5 mL) was added to 110.4_1b (300 mg, 0.65 mmol) at RT. After stirring for 2 h, the reaction mixture was concentrated under reduced pressure. To the resulting residue dissolved in CH₂Cl₂(3 mL) was a solution of 3,5-dichloro-N-neopentylaniline (217 mg, 0.93 mmol) in CH₂Cl₂ (2 mL). After stirring for 16 h at 55° C., the reaction mixture was quenched with water (10 mL) and extracted with CH₂Cl₂ (2×20 mL). The combined organic layer was washed with brine, dried over anhydrous Na₂SO₄, filtered and concentrated under reduced pressure. The residue was purified by column chromatography (40 g silica gel cartridge, gradient 15% EtOAc/pet ether) to afford 111.1b (125 mg, 30%) as a solid.

¹H NMR (400 MHz, DMSO-d₆): 11.10/11.04 (s, 1H), 8.13 (d, J=2.0 Hz, 1H), 7.71-7.44 (m, 4H), 5.46-5.36 (m, 1H), 5.18-5.05 (m, 2H), 4.28-4.19 (m, 2H), 4.14 (d, J=7.6 Hz, 1H), 3.87-3.78 (m, 2H), 3.70 (t, J=7.2 Hz, 1H), 3.61 (d, J=14.0 Hz, 1H), 3.28-3.26 (m, 1H), 2.67-2.57 (m, 1H), 2.44-2.38 (m, 1H), 2.16-2.07 (m, 1H), 0.84 (s, 9H); LCMS 94.5%, m/z [M+H]⁺=674.0.

### Synthesis of 111

To a stirred solution of 111.1b (125 mg, 0.18 mmol) in THF (3 mL) were added aniline (17 mg, 0.18 mmol) and by Pd(PPh₃)₄ (42 mg, 0.03 mmol) at RT. After stirring for 2 h at RT, the reaction mixture was concentrated under reduced pressure. The residue was purified by prep-HPLC (XSELECT-C18 (150×30), 5 μ; A: 0.1% Formic Acid in H₂O; B: acetonitrile; Gradient: (Time in minutes/%B): 0/70, 8/95, 12/98 at 24 mL/min) to afford 111 (40 mg, 34%) as a solid.

[α]²⁵D+9.70 (c 0.5, MeOH); ¹H NMR (500 MHz, DMSO-d₆): 12.69 (br s, 1H), 11.05/10.99 (s, 1H), 8.27 (d, J=2.0 Hz, 1H), 7.82-7.43 (m, 4H), 4.05 (d, J=7.5 Hz, 1H), 4.01 (d, J=14 Hz, 1H), 3.84-3.80 (m, 1H), 3.60 (dd, J=7.5 Hz, J=6.5 Hz, 1H), 3.45 (d, J=14.0 Hz, 1H), 3.29-3.27 (m, 1H), 2.60-2.54 (m, 1H), 2.38-2.34 (m, 1H), 2.10-2.05 (m, 1H), 0.85/0.82 (s, 9H); ¹⁹F NMR (376.49 MHz, DMSO-d₆): −89.21 (d, J=226 Hz), −97.18 (d, J=226 Hz); LCMS 95.9%, m/z [M+H]⁺=634.0; Chiral purity: 97.6%.

TABLE 5

| | | | M/Z | M/Z | |
|---|---|---|---|---|---|
| Ex. | SM | Compound | (M + H)+ | (M − H)− | 1H NMR |
| 112 | 1-(1-(3-(tert-butyl) phenyl)cyclopropyl)-N- methylmethanamine | 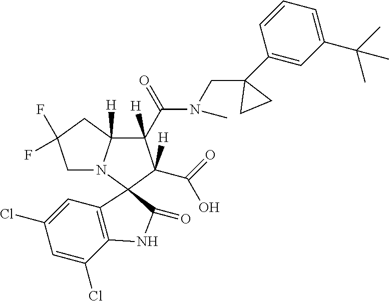 [SMILES: CN(CC1(c2cccc(C(C)(C)C)c2)CC1)C(=O)[C@H]1[C@H]2CC(F)(F)CN2[C@]2(C(=O)Nc3c(Cl)cc(Cl)cc32)[C@H]1C(=O)O] | 620.2 | | (500 MHz, DMSO-d₆) (Exist in rotameric form): 12.21 (br s, 1H), 10.95/10.90 (br s, 1H), 8.06 (s, 1H), 7.43-7.39 (m, 2H), 7.27-7.19 (s, 3H), 4.50-4.30 (m, 1H), 3.96-3.88 (m, 2H), 3.65-3.55 (m, 1H), 3.30-3.15 (m, 1H), 2.97-2.89 (m, 2H), 2.89/2.80 (s, 3H), 2.55-2.40 (m, 1H), 1.90-1.80 (m, 1H), 1.28/1.26 (s, 9H), 1.01-0.93 (m, 2H), 0.90-0.79 (m, 1H), 0.67-0.62 (m, 1H). |
| 113 | quinolin-2-amine | 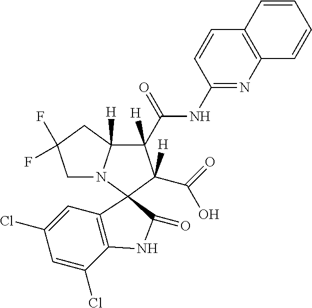 [SMILES: O=C(Nc1ccc2ccccc2n1)[C@H]1[C@H]2CC(F)(F)CN2[C@]2(C(=O)Nc3c(Cl)cc(Cl)cc32)[C@H]1C(=O)O] | 547 | | (500 MHz, DMSO-d₆): 12.77 (br s, 1H), 11.17 (s, 1H), 10.97 (br s, 1H), 8.41-8.35 (m, 2H), 7.94 (d, J = 8.0 Hz, 1H), 7.84 (d, J = 8.5 Hz, 1H), 7.76-7.73 (m, 1H), 7.64-7.62 (m, 1H), 7.61-7.53 (m, 1H), 7.34 (s, 1H), 4.04-3.94 (m, 3H), 2.98-2.92 (m, 1H), 2.77-2.50 (m, 3H). |
| 114 | 6- (trifluoromethyl)pyridin- 2-amine | 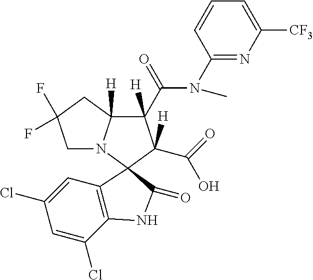 [SMILES: CN(C(=O)[C@H]1[C@H]2CC(F)(F)CN2[C@]2(C(=O)Nc3c(Cl)cc(Cl)cc32)[C@H]1C(=O)O)c1cccc(C(F)(F)F)n1] | | 577.2 | (500 MHz, DMSO-d₆): 12.39 (br s, 1H), 11.04 (s, 1H), 8.21 (br s, 2H), 7.83-7.48 (m, 3H), 3.99 (br s, 2H), 3.79 (br s, 1H), 3.41 (br s, 3H), 3.17-3.08 (m, 1H), 2.69-2.62 (m, 1H), 2.49-2.46 (m, 1H), 2.20-1.95 (m, 1H). |
| 115 | naphthalen-2-amine | 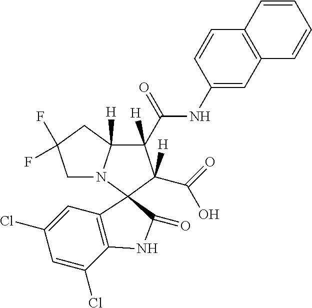 [SMILES: O=C(Nc1ccc2ccccc2c1)[C@H]1[C@H]2CC(F)(F)CN2[C@]2(C(=O)Nc3c(Cl)cc(Cl)cc32)[C@H]1C(=O)O] | 546 | | (500 MHz, DMSO-d₆): 12.44 (br s, 1H), 11.06 (br s, 1H), 10.45 (br s, 1H), 8.28-8.20 (m, 2H), 7.91-7.83 (m, 3H), 7.64-7.60 (m, 1H), 7.57-7.41 (m, 3H), 4.13-4.09 (m, 2H), 3.67-3.65 (m, 1H), 3.32-3.17 (m, 1H), 2.70-2.60 (m, 1H), 2.50-2.41 (m, 1H), 2.14-2.07 (m, 1H). |
| 116 | 3,5-dichloro-N- cyclopropylaniline | 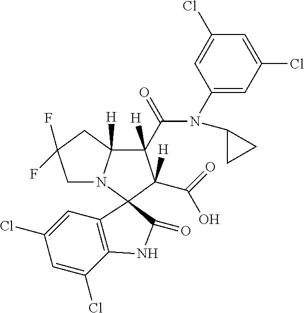 [SMILES: O=C(O)[C@H]1[C@@H](C(=O)N(c2cc(Cl)cc(Cl)c2)C2CC2)[C@H]2CC(F)(F)CN2[C@]12C(=O)Nc1c(Cl)cc(Cl)cc12] | 604.1 | | (500 MHz, DMSO-d₆) (Exist in rotameric form): 12.60 (br s, 1H), 11.05/11.00 (s, 1H), 8.28/7.93 (d, J = 2.0 Hz, 1H), 7.69-7.32 (m, 4H), 4.62-4.60 (m, 1H), 4.24-3.80 (m, 2H), 3.32-3.12 (m, 2H), 2.64-2.57 (m, 1H), 2.50-2.36 (m, 1H), 2.10-2-02 (m, 1H), 1.01-0.99 (m, 2H). 0.71-0.53 (m, 2H). |
| 117 | naphthalen-1-amine | 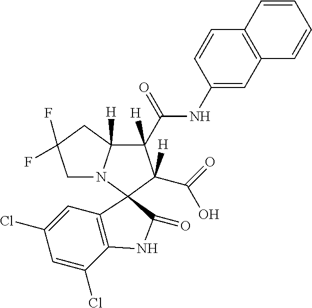 [SMILES: O=C(Nc1ccc2ccccc2c1)[C@H]1[C@H]2CC(F)(F)CN2[C@]2(C(=O)Nc3c(Cl)cc(Cl)cc32)[C@H]1C(=O)O] | 546.2 | | (500 MHz, DMSO-d₆): 12.56 (br s, 1H), 11.04 (s, 1H), 10.31 (br s, 1H), 8.18 (t, J = 5.0 Hz, 1H), 8.13-8.10 (m, 1H), 7.98-7.96 (m, 1H), 7.83 (d, J = 8.0 Hz, 1H), 7.63-7.43 (m, 4H), 7.43 (s, 1H), 4.19-4.15 (m, 2H), 3.83 (t, J = 7.0 Hz, 1H), 3.21-3.17 (m, 1H), 2.65-2.61 (m, 2H), 2.35-2.15 (m, 1H). |
| 118 | 2- methylbenzo[d]thiazol- 6-amine | 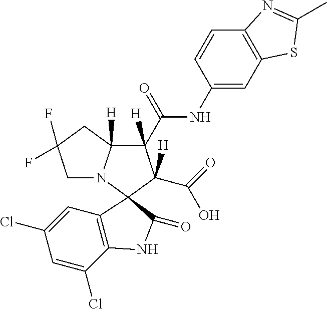 [SMILES: Cc1nc2ccc(NC(=O)[C@H]3[C@H]4CC(F)(F)CN4[C@]4(C(=O)Nc5c(Cl)cc(Cl)cc54)[C@H]3C(=O)O)cc2s1] | | 565.1 | (500 MHz, DMSO-d₆): 12.75 (br s, 1H), 11.16 (br s, 1H), 10.43 (br s, 1H), 8.45 (s, 1H), 7.86 (d, J = 8.5 Hz, 1H), 7.62-7.53 (m, 2H), 7.21 (br s, 1H), 4.05-3.90 (m, 2H), 3.64 (t, J = 10.5 Hz 1H), 2.95-2.94 (m, 1H), 2.77 (s, 3H), 2.69-2.56 (m, 1H), 2.50-2.36 (m, 2H). |
| 119 | isoquinolin-3-amine | 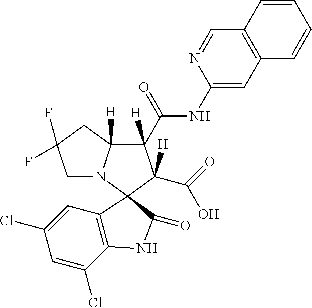 [SMILES: O=C(Nc1cc2ccccc2cn1)[C@H]1[C@H]2CC(F)(F)CN2[C@]2(C(=O)Nc3c(Cl)cc(Cl)cc32)[C@H]1C(=O)O] | 547.2 | | (500 MHz, DMSO-d₆): 12.74 (br s, 1H), 11.18 (br s, 1H), 10.76 (br s, 1H), 9.18 (br s, 1H), 8.51 (s, 1H), 8.07 (d, J = 7.5 Hz, 1H), 7.92-7.90 (m, 1H), 7.72 (t, J = 7.5 Hz, 1H), 7.61-7.51 (m, 2H), 7.34 (br s, 1H), 4.05-3.93 (m, 3H), 2.98-2-92 (m, 1H), 2.74 (m, 1H), 2.52-2.50 (m, 2H). |
| 120 | quinolin-6-amine | 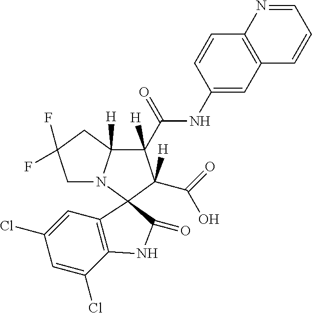 [SMILES: O=C(Nc1ccc2ncccc2c1)[C@H]1[C@H]2CC(F)(F)CN2[C@]2(C(=O)Nc3c(Cl)cc(Cl)cc32)[C@H]1C(=O)O] | 547.2 | | (500 MHz, DMSO-d₆): 12.49 (br s, 1H), 11.02 (br s, 1H), 10.90-10.50 (br s, 1H), 8.79 (dd, J = 4.0, 1.5 Hz, 1H), 8.38-8.35 (m, 2H), 8.21-8.09 (m, 1H), 7.99 (d, J = 9.0 Hz, 1H), 7.83 (dd, J = 9.0, 2.0 Hz, 1H), 7.50-7.41 (m, 2H), 4.22-4.00 (m, 2H), 3.72-3.56 (m, 1H), 3.26-3.18 (m, 1H), 2.67-2.65 (m, 1H), 2.50-2.40 (m, 1H), 2.27-2.03 (m, 1H). |
| 121 | [1,1′-biphenyl]-2-amine | 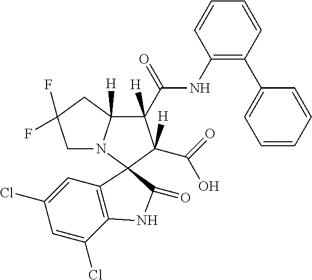 [SMILES: O=C(Nc1ccccc1-c1ccccc1)[C@H]1[C@H]2CC(F)(F)CN2[C@]2(C(=O)Nc3c(Cl)cc(Cl)cc32)[C@H]1C(=O)O] | 572.2 | | (500 MHz, DMSO-d₆): 12.48 (br s, 1H), 11.01 (br s, 1H), 9.63 (br s, 1H), 8.15 (s, 1H), 7.51-7.34 (m, 10H), 4.02 (d, J = 6.5 Hz, 1H), 3.98-3.94 (m, 1H), 3.53-3.43 (m, 1H), 3.19-3.11 (m, 1H), 2.57-2.50 (m, 1H), 2.09-1.98 (m, 1H), 1.91-1.72 (m, 1H). |
| 122 | isoquinolin-1-amine | 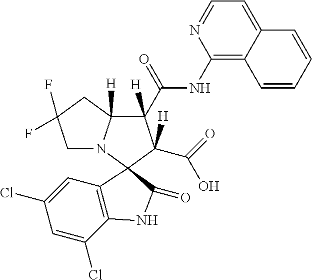 [SMILES: O=C(Nc1nccc2ccccc12)[C@H]1[C@H]2CC(F)(F)CN2[C@]2(C(=O)Nc3c(Cl)cc(Cl)cc32)[C@H]1C(=O)O] | 547.1 | | (500 MHz, DMSO-d₆): 12.83 (br s, 1H), 11.18 (s, 1H), 10.74 (br s, 1H), 8.35 (br s, 1H), 8.01-7.99 (m, 2H), 7.81-7.79 (m, 2H), 7.69-7.65 (m, 1H), 7.62 (s, 1H), 7.28 (br s, 1H), 4.09-3.91 (m, 3H), 3.17-2.95 (m, 1H), 2.85-2.56 (m, 3H). |
| 123 | 1-methyl-1H- pyrazolo[3,4-b]pyridin- 3-amine | 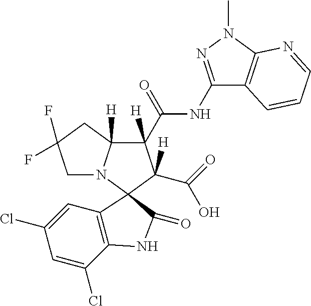 [SMILES: Cn1nc(NC(=O)[C@H]2[C@H]3CC(F)(F)CN3[C@]3(C(=O)Nc4c(Cl)cc(Cl)cc43)[C@H]2C(=O)O)c2cccnc21] | 551 | | (500 MHz, MeOH-d₄): 8.52 (d, J = 1.5 Hz, 1H), 8.52-8.47 (dd, J = 12.5, 1.5 Hz, 1H), 8.22 (d, J = 2.0 Hz, 1H), 7.30 (d, J = 2.0 Hz, 1H), 7.20-7.17 (dd, J = 8.5, 4.5 Hz, 1H), 4.29-4.20 (m, 2H), 4.06 (s, 3H), 3.70 (t, J = 7.0 Hz, 1H), 3.24-3.18 (m, 1H), 2.75-2.68 (m, 1H), 2.49-2.42 (m, 1H), 2.41-2.24 (m, 1H). |
| 124 | [1,1′-biphenyl]-3-amine | 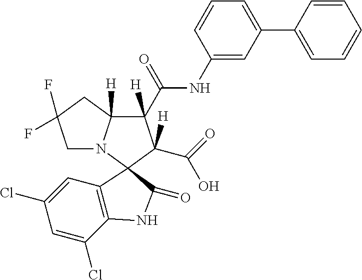 [SMILES: O=C(Nc1cccc(-c2ccccc2)c1)[C@H]1[C@H]2CC(F)(F)CN2[C@]2(C(=O)Nc3c(Cl)cc(Cl)cc32)[C@H]1C(=O)O] | 572 | | (500 MHz, DMSO-d₆): 12.43 (br s, 1H), 11.07 (s, 1H), 10.35 (s, 1H), 8.20 (d, J = 1.5 Hz, 1H), 7.86 (s, 1H), 7.68-7.62 (m, 3H), 7.51-7.39 (m, 6H), 4.16-4.09 (m, 2H), 3.64 (t, J = 7.0 Hz, 1H), 3.27-3.19 (m, 1H), 2.69-2.62 (m, 1H), 2.48-2.44 (m, 1H), 2.11-2.05 (m, 1H). |
| 125 | benzo[d]thiazol-2-amine | 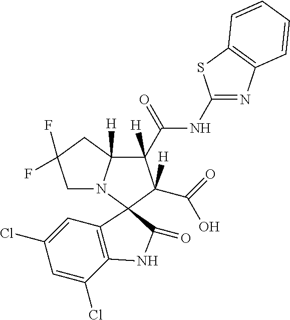 [SMILES: O=C(Nc1nc2ccccc2s1)[C@H]1[C@H]2CC(F)(F)CN2[C@]2(C(=O)Nc3c(Cl)cc(Cl)cc32)[C@H]1C(=O)O] | 553 | | (500 MHz, DMSO-d₆) δ: 12.78 (br s, 1H), 10.89 (br s, 1H), 7.97 (d, J = 7.5 Hz, 1H), 7.74 (d, J = 7.0 Hz, 1H), 7.53-7.42 (m, 3H), 7.32-7.08 (m, 2H), 4.16 (br s, 1H), 3.93 (t, J = 9.0 Hz, 1H), 3.78 (br s, 1H), 2.89 (br s, 1H), 2.67-2.36 (m, 3H). |
| 126 | quinoxalin-5-amine | 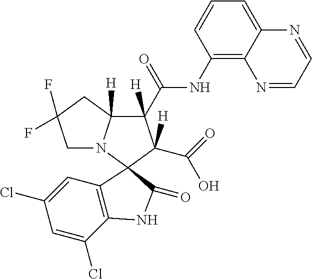 [SMILES: O=C(Nc1cccc2nccnc12)[C@H]1[C@H]2CC(F)(F)CN2[C@]2(C(=O)Nc3c(Cl)cc(Cl)cc32)[C@H]1C(=O)O] | 548.2 | | (500 MHz, DMSO-d₆): 12.45 (br s, 1H), 11.05 (br s, 1H), 10.63 (s, 1H), 9.05 (d, J = 1.5 Hz, 1H), 8.96 (d, J = 1.5 Hz, 1H), 8.70 (d, J = 7.5 Hz, 1H), 8.10 (br s, 1H), 7.91 (t, J = 8.0, 1H), 7.84 (d, J = 8.5 Hz, 1H), 7.46 (s, 1H), 4.15-4.02 (m, 3H), 3.27-3.21 (m, 1H), 2.69-2.62 (m, 1H), 2.50-2.43 (m, 1H), 2.22-2.07 (m, 1H). |
| 127 | quinolin-8-amine | 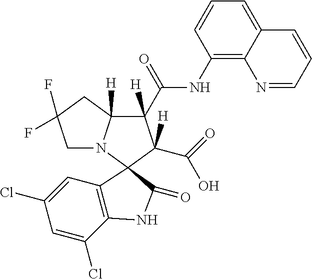 [SMILES: O=C(Nc1cccc2cccnc12)[C@H]1[C@H]2CC(F)(F)CN2[C@]2(C(=O)Nc3c(Cl)cc(Cl)cc32)[C@H]1C(=O)O] | 547 | | (500 MHz, DMSO-d₆): 12.77 (br s, 1H), 11.14 (br s, 1H), 10.53 (br s, 1H), 9.01 (d, J = 2.0 Hz, 1H), 8.73 (d, J = 7.0 Hz, 1H), 8.44 (d, J = 8.0 Hz, 1H), 7.72-7.59 (m, 5H), 4.38-4.26 (m, 1H), 4.06-3.99 (m, 2H), 2.93-2.73 (m, 3H), 2.60-2.50 (m, 1H). |
| 128 | benzo[d]thiazol-5-amine | 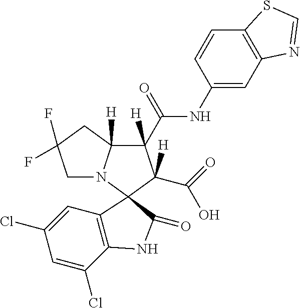 [SMILES: O=C(Nc1ccc2scnc2c1)[C@H]1[C@H]2CC(F)(F)CN2[C@]2(C(=O)Nc3c(Cl)cc(Cl)cc32)[C@H]1C(=O)O] | 553 | | (500 MHz, DMSO-d₆): δ 12.47 (br s, 1H), 11.01 (br s, 1H), 10.64 (br s, 1H), 9.40 (s, 1H), 8.50 (d, J = 2.0 Hz, 1H), 8.20-8.15 (m, 1H), 8.11 (d, J = 9.0 Hz, 1H), 7.62 (dd, J = 8.5, 1.5 Hz, 1H), 7.46 (s, 1H), 4.14-4.08 (m, 2H), 3.62-3.58 (m, 1H), 3.32-3.15 (m, 1H), 2.67-2.61 (m, 1H), 2.50-2.36 (m, 1H), 2.14-2.07 (m, 1H). |
| 129 | 2,3-dihydrobenzo[b] [1,4]dioxin-5-amine | 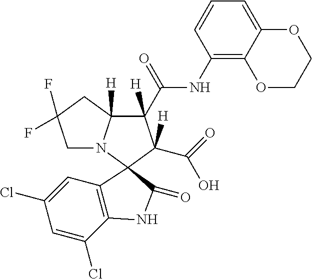 [SMILES: O=C(Nc1cccc2c1OCCO2)[C@H]1[C@H]2CC(F)(F)CN2[C@]2(C(=O)Nc3c(Cl)cc(Cl)cc32)[C@H]1C(=O)O] | 554 | | (500 MHz, DMSO-d₆): 12.45 (br s, 1H), 11.05 (s, 1H), 9.51 (s, 1H), 8.14 (s, 1H), 7.47 (d, J = 2.0 Hz, 1H), 7.43 (d, J = 7.5 Hz 1H), 6.81 (dd, J = 8.5 Hz, 8.0 Hz, 1H), 6.66 (dd, J = 8.5 Hz, 2.0 Hz, 1H), 4.31-4.24 (m, 4H), 4.09-4.05 (m, 1H), 4.00 (d, J = 7.5 Hz, 1H), 3.79 (t, J = 7.0 Hz, 1H), 3.22-3.14 (m, 1H), 2.65-2.59 (m, 1H), 2.50-2.36 (m, 1H), 2.18-2.07 (m 1H). |
| 130 | 5-fluoro-1H-indazol-3- amine | 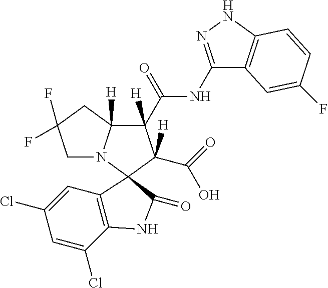 [SMILES: O=C(Nc1n[nH]c2ccc(F)cc12)[C@H]1[C@H]2CC(F)(F)CN2[C@]2(C(=O)Nc3c(Cl)cc(Cl)cc32)[C@H]1C(=O)O] | | 552 | (500 MHz, DMSO-d₆): 8.26 (dd, J = 9.0, 4.5 Hz, 1H), 7.76 (dd, J = 8.5, 2.5 Hz, 1H), 7.56 (br s, 1H), 7.47 (dt, J = 8.5, 2.5 Hz, 1H), 7.14 (br s, 1H), 6.63 (br s, 2H), 4.54-4.49 (m, 1H), 4.16-4.14 (m, 1H), 3.95-3.94 (m, 1H), 2.95-2.90 (m, 1H), 2.81-2.76 (m, 1H), 2.69-2.60 (m, 2H). |
| 131 | 5,6,7,8- tetrahydronaphthalen-1- amine | 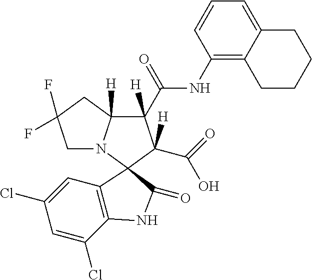 [SMILES: O=C(Nc1cccc2c1CCCC2)[C@H]1[C@H]2CC(F)(F)CN2[C@]2(C(=O)Nc3c(Cl)cc(Cl)cc32)[C@H]1C(=O)O] | | 548.1 | (500 MHz, DMSO-d₆): 12.45 (br s, 1H), 11.02 (s, 1H), 9.52 (br s, 1H), 8.14 (br s, 1H), 7.44 (s, 1H), 7.28-7.24 (m, 1H), 7.10 (dd, J = 8.0, 7.5 Hz, 1H), 6.95 (d, J = 8.0 Hz, 1H), 4.14-4.07 (m, 2H), 3.65 (t, J = 6.5 Hz, 1H), 3.26-3.18 (m, 1H), 2.74-2.57 (m, 6H), 2.37-2.21 (m, 1H), 1.75-1.70 (m, 4H). |
| 132 | 3,5-dichloro-N-(2- methoxyethyl)anilin | 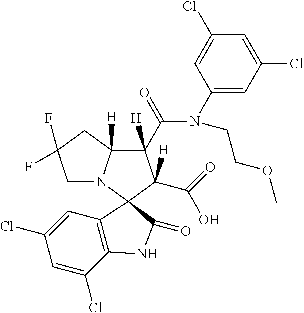 [SMILES: COCCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] | 622.2 | | (500 MHz, DMSO-d₆) (Exist in rotameric form): 12.46 (br s, 1H), 11.02 (br s, 1H), 8.21/8.13 (br s, 1H), 7.97-7.03 (m, 4H), 4.16-3.97 (m, 4H), 3.94 (s, 3H), 3.52-3.32 (m, 3H), 3.32-3.17 (m, 1H), 2.66-2.57 (m, 1H), 2.50-2.36 (m, 1H), 2.17-2.07 (m, 1H). |
| 133 | 6-methoxy-N-methyl-4- (trifluoromethyl)pyridin- 2-amine | 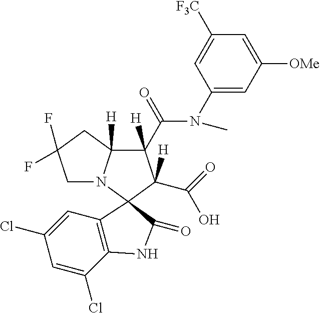 [SMILES: COc1cc(N(C)C(=O)[C@H]2[C@H]3CC(F)(F)CN3[C@]3(C(=O)Nc4c(Cl)cc(Cl)cc43)[C@H]2C(=O)O)cc(C(F)(F)F)c1] | 609.2 | | (500 MHz, MeOD): 8.29 (br s, 1H), 7.69-6.90 (m, 3H), 4.14-3.91 (m, 3H), 3.99 (s, 3H), 3.42 (m, 3H), 3.30-3.16 (m, 1H), 2.70-2.65 (m, 1H), 2.39-2.24 (m, 2H). |
| 134 | 5-phenylpyrazin-2- amine | 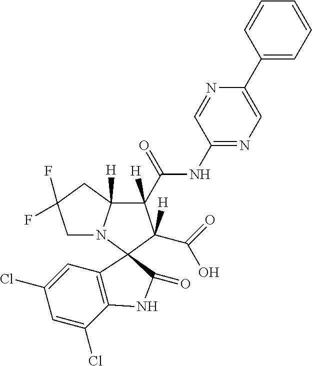 [SMILES: O=C(Nc1cnc(-c2ccccc2)cn1)[C@H]1[C@H]2CC(F)(F)CN2[C@]2(C(=O)Nc3c(Cl)cc(Cl)cc32)[C@H]1C(=O)O] | 574 | | (500 MHz, DMSO-d₆): 12.50 (s, 1H), 11.17 (s, 1H), 11.10 (s, 1H), 9.43 (s, 1H), 9.04 (d, J = 1.0 Hz, 1H), 8.14-8.11 (m, 3H), 7.55-7.46 (m, 4H), 4.11-4.08 (m, 2H), 3.83-3.82 (m, 1H), 3.22-3.16 (m, 1H), 2.70-2.64 (m, 1H), 2.47-2.43 (m, 1H), 2.21-1.95 (m, 1H). |
| 135 | 1,7-naphthyridin-8- amine | 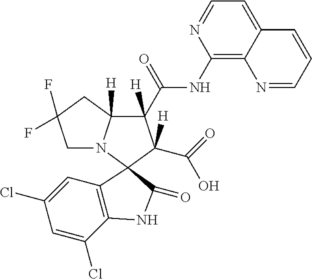 [SMILES: O=C(Nc1nccc2cccnc12)[C@H]1[C@H]2CC(F)(F)CN2[C@]2(C(=O)Nc3c(Cl)cc(Cl)cc32)[C@H]1C(=O)O] | 548.1 | | (500 MHz, DMSO-d₆): 12.72 (br s, 1H), 11.45-10.70 (br s, 2H), 9.06 (br s, 1H), 8.45 (d, J = 8.5 Hz, 1H), 8.34 (d, J = 6.0 Hz, 1H), 7.87-7.85 (m, 1H), 7.66-7.63 (m, 1H), 7.55 (s, 2H), 4.29-4.22 (m, 1H), 4.10-4.06 (m, 1H), 3.95-3.90 (m, 1H), 2.93-2.88 (m, 1H), 2.79-2.72 (m, 2H), 2.50-2.49 (m, 1H). |
| 136 | 1-methyl-1H-indol-6- amine | 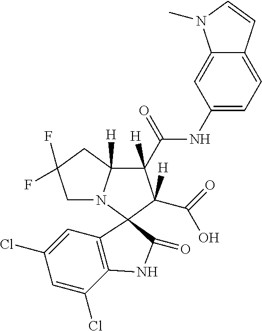 [SMILES: Cn1ccc2ccc(NC(=O)[C@H]3[C@H]4CC(F)(F)CN4[C@]4(C(=O)Nc5c(Cl)cc(Cl)cc54)[C@H]3C(=O)O)cc21] | 549.1 | | (500 MHz, DMSO-d₆): 13.0-12.0 (brs, 1H), 11.20-10.60 (br s, 1H), 7.93 (s, 1H), 7.51 (br s. 1H), 7.45 (d, J = 8.0 Hz, 1H), 7.31 (br s, 1H), 7.24 (d, J = 2.5 Hz, 1H), 7.01-6.98 (m, 2H), 6.34 (d, J = 2.5 Hz, 1H), 4.12 (br s. 1H), 3.73 (s, 3H), 3.69-3.64 (m, 2H), 2.89-2.63 (m, 2H), 2.41-2.36 (m, 2H). |
| 137 | 1-methyl-1H-indol-5- amine | 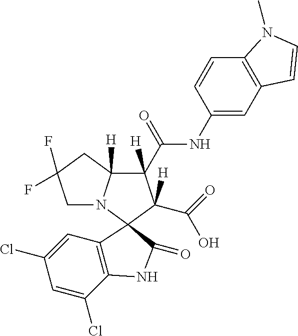 [SMILES: Cn1ccc2cc(NC(=O)[C@H]3[C@H]4CC(F)(F)CN4[C@]4(C(=O)Nc5c(Cl)cc(Cl)cc54)[C@H]3C(=O)O)ccc21] | 549.3 | | (500 MHz, DMSO-d₆): 12.36 (br s, 1H), 11.04 (s, 1H), 10.04 (br s, 1H), 8.25 (s, 1H), 7.86 (s, 1H), 7.47 (d, J = 1.5 Hz, 1H), 7.39 (d, J = 8.5 Hz, 1H), 7.31 (d, J = 3.0 Hz, 1H), 7.26-7.24 (m, 1H), 6.41 (d, J = 2.5 Hz, 1H), 4.15-4.10 (m, 1H), 4.06 (d, J = 7.5 Hz, 1H), 3.78 (s, 3H), 3.60 (t, J = 7.0 Hz, 1H), 3.28-3.20 (m, 1H), 2.67-2.60 (m, 1H), 2.50-2.44 (m, 1H), 2.20-2.05 (m, 1H). |
| 138 | 1-methyl-1H-indazol-5- amine | 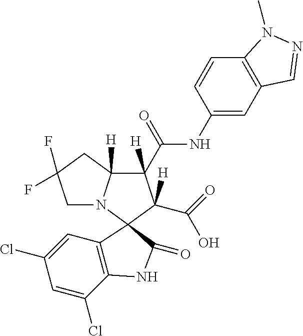 [SMILES: Cn1ncc2cc(NC(=O)[C@H]3[C@H]4CC(F)(F)CN4[C@]4(C(=O)Nc5c(Cl)cc(Cl)cc54)[C@H]3C(=O)O)ccc21] | | 548 | (500 MHz, DMSO-d₆): 12.72 (br s, 1H), 11.15 (br s, 1H), 10.24 (br s, 1H), 8.16 (s, 1H), 8.00 (s, 1H), 7.71-7.36 (m, 3H), 7.22 (br s, 1H), 4.02 (s, 3H), 4.02-3.90 (m, 2H), 3.62 (t, J = 11.5 Hz, 1H), 2.95-2.94 (m, 1H), 2.78-2.76 (m, 1H), 2.64-2.55 (m, 1H), 2.50-2.36 (m, 1H). |
| 139 | 1-methyl-1H- benzo[d]imidazol-2- amine | 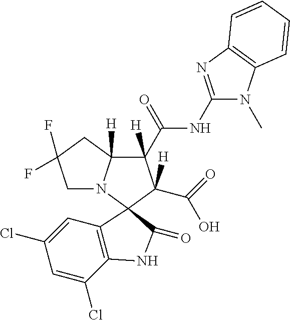 [SMILES: Cn1c(NC(=O)[C@H]2[C@H]3CC(F)(F)CN3[C@]3(C(=O)Nc4c(Cl)cc(Cl)cc43)[C@H]2C(=O)O)nc2ccccc21] | 550 | | (500 MHz, DMSO-d₆): 14.24 (br s, 1H), 12.35 (br s, 1H), 10.98 (br s, 1H), 7.49-7.45 (m, 4H), 7.23-7.19 (m, 2H), 4.01-3.75 (m, 3H), 3.61 (s, 3H), 2.91-2.80 (m, 2H), 2.77-2.66 (m, 1H), 2.65-2.50 (m, 1H). |
| 140 | quinazolin-6-amine | 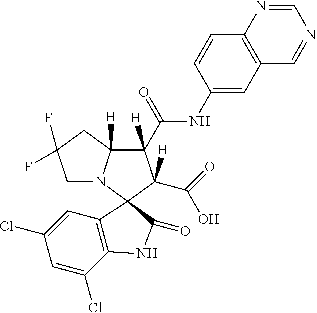 [SMILES: O=C(Nc1ccc2ncncc2c1)[C@H]1[C@H]2CC(F)(F)CN2[C@]2(C(=O)Nc3c(Cl)cc(Cl)cc32)[C@H]1C(=O)O] | 548 | | (500 MHz, DMSO-d₆): 12.56 (br s, 1H), 11.04 (br s, 1H), 9.62 (s, 1H), 9.20 (s, 1H), 8.58 (d, J = 2.0 Hz, 1H), 8.11-8.01 (m, 3H), 7.47 (s, 1H), 4.15-4.10 (m, 2H), 3.68-3.65 (m, 1H), 3.26-3.16 (m, 1H), 2.69-2.63 (m, 1H), 2.49-2.40 (m, 1H), 2.23-2.07 (m, 1H). |
| 141 | 1-methyl-1H-indol-4- amine | 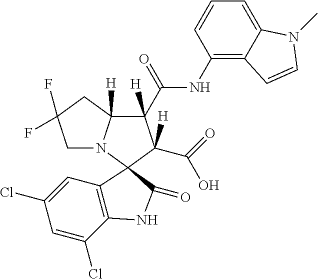 [SMILES: Cn1ccc2c(NC(=O)[C@H]3[C@H]4CC(F)(F)CN4[C@]4(C(=O)Nc5c(Cl)cc(Cl)cc54)[C@H]3C(=O)O)cccc21] | 549 | | (500 MHz, DMSO-d₆): 12.40 (br s, 1H), 11.05 (s, 1H), 9.92 (br s, 1H), 8.21 (s, 1H), 7.50-7.46 (m, 2H), 7.29 (d, J = 3.0 Hz, 1H), 7.25 (d, J = 8.0 Hz, 1H), 7.14 (t, J = 8.0 Hz, 1H), 6.66 (d, J = 2.5 Hz, 1H), 4.16-4.11 (m, 1H), 4.09 (d, J = 7.5 Hz, 1H), 3.82 (t, J = 7.0 Hz, 1H), 3.79 (s, 3H), 3.24-3.16 (m, 1H), 2.67-2.60 (m, 1H), 2.55-2.50 (m, 1H), 2.13-2.07 (m, 1H). |
| 142 | 1-methyl-1H-imidazol- 4-amine | 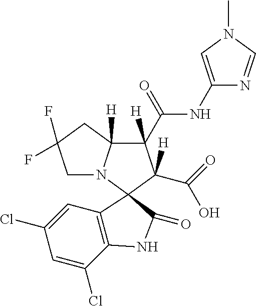 [SMILES: Cn1cnc(NC(=O)[C@H]2[C@H]3CC(F)(F)CN3[C@]3(C(=O)Nc4c(Cl)cc(Cl)cc43)[C@H]2C(=O)O)c1] | 500.2 | | (500 MHz, DMSO-d₆): 10.84 (br s, 1H), 10.67 (br s, 1H), 8.14-8.10 (m, 1H), 7.38-7.37 (m, 2H), 7.26 (s, 1H), 4.00-3.97 (m, 1H), 3.88-3.81 (m, 1H), 3.63 (s, 3H), 3.54-3.49 (m, 1H), 3.24-3.10 (m, 1H), 2.58-2.50 (m, 1H), 2.36-2.17 (m, 2H). |
| 143 | 2-methyl-2H-indazol-6- amine | 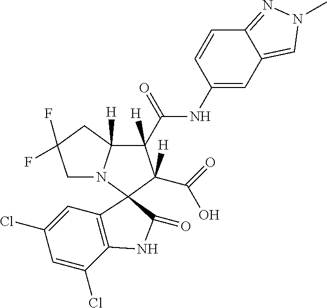 [SMILES: Cn1cc2cc(NC(=O)[C@H]3[C@H]4CC(F)(F)CN4[C@]4(C(=O)Nc5c(Cl)cc(Cl)cc54)[C@H]3C(=O)O)ccc2n1] | 550.2 | | (500 MHz, DMSO-d₆): 12.41 (s, 1H), 11.06 (s, 1H), 10.21 (s, 1H), 8.27 (s, 2H), 8.05 (s, 1H), 7.65 (d, J = 9.0 Hz, 1H), 7.49 (d, J = 2.0 Hz, 1H), 7.09 (dd, J = 9.0, 2.0 Hz, 1H), 4.15 (s, 3H), 4.13-4.09 (m, 2H), 3.63 (t, J = 7.0 Hz, 1H), 3.27-3.22 (m, 1H), 2.66-2.63 (m, 1H), 2.45-2.44 (m, 1H), 2.10-1.95 (m, 1H). |
| 144 | quinazolin-4-amine | 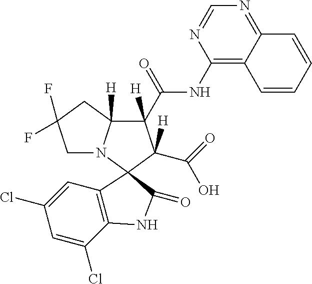 [SMILES: O=C(Nc1ncnc2ccccc12)[C@H]1[C@H]2CC(F)(F)CN2[C@]2(C(=O)Nc3c(Cl)cc(Cl)cc32)[C@H]1C(=O)O] | 548 | | (500 MHz, DMSO-d₆): 14.57 (br s, 1H), 10.78 (br s, 1H), 8.94 (br s, 1H), 8.46 (br s, 1H), 7.96-7.91 (m, 2H), 7.77-7.42 (m, 4H), 4.44-4.22 (m, 1H), 4.04-3.90 (m, 1H), 3.72-3.54 (m, 1H), 3.29-3.24 (m, 1H), 2.89-2.84 (m, 1H), 2.68-2.64 (m, 1H), 2.55-2.40 (m, 1H). |
| 145 | 3,5-dichloro-N- (cyclopentylmethyl) aniline | 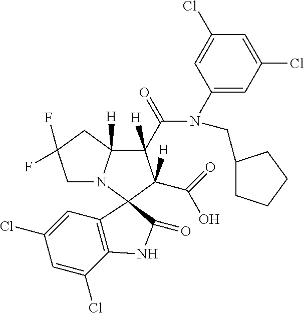 [SMILES: O=C(O)[C@H]1[C@@H](C(=O)N(CC2CCCC2)c2cc(Cl)cc(Cl)c2)[C@H]2CC(F)(F)CN2[C@]12C(=O)Nc1c(Cl)cc(Cl)cc12] | 646 | | (500 MHz, DMSO-d₆) (Exist in rotameric form): 12.56 (s, 1H), 11.04/10.98 (s, 1H), 8.28 (d, J = 2.0 Hz, 1H), 8.13-7.36 (m, 4H), 4.02-3.93 (m, 2H), 3.82-3.80 (m, 1H), 3.52-3.47 (m, 2H), 3.34-3.26 (m, 1H), 2.51-2.49 (m, 1H), 2.49-2.36 (m, 1H), 2.09-2.07 (m, 1H), 1.92-1.89 (m, 1H), 1.65-1.58 (m, 4H), 1.48-1.45 (m, 2H), 1.37-1.35 (m, 1H), 1.14-1.13 (m, 1H). |
| 146 | 1-methyl-1H- benzo[d]imidazol-5- amine | 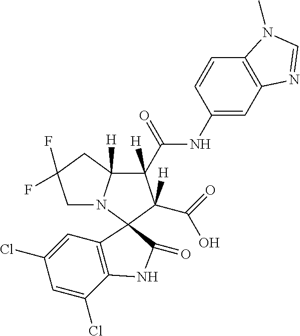 [SMILES: Cn1cnc2cc(NC(=O)[C@H]3[C@H]4CC(F)(F)CN4[C@]4(C(=O)Nc5c(Cl)cc(Cl)cc54)[C@H]3C(=O)O)ccc21] | 550.1 | | |
| 147 | quinoxalin-6-amine | 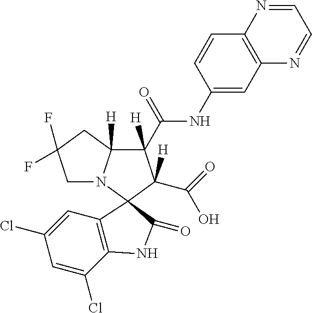 [SMILES: O=C(Nc1ccc2nccnc2c1)[C@H]1[C@H]2CC(F)(F)CN2[C@]2(C(=O)Nc3c(Cl)cc(Cl)cc32)[C@H]1C(=O)O] | 548 | | (500 MHz, DMSO-d₆): 12.80 (br s, 1H), 11.25 (br s, 1H), 10.77 (br s, 1H), 8.90 (d, J = 1.5 Hz, 1H), 8.84 (s, 1H), 8.62-8.46 (m, 1H), 8.09 (d, J = 9.0 Hz, 1H), 7.95 (m, 1H), 7.64-7.60 (m, 1H), 7.22 (br s, 1H), 4.15-3.96 (m, 2H), 3.70-3.60 (m, 1H), 2.96-2.94 (m, 1H), 2.79-2.76 (m, 1H), 2.72-2.60 (m, 1H), 2.50-2.40 (m, 1H). |
| 148 | 3,5-dichloro-N- (cyclobutylmethyl) aniline | 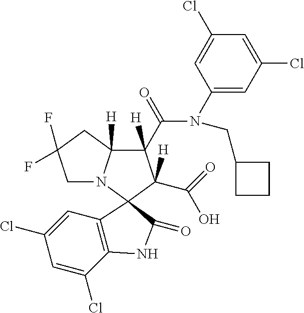 [SMILES: O=C(O)[C@H]1[C@@H](C(=O)N(CC2CCC2)c2cc(Cl)cc(Cl)c2)[C@H]2CC(F)(F)CN2[C@]12C(=O)Nc1c(Cl)cc(Cl)cc12] | 632 | | (500 MHz, DMSO-d₆) (Exist in rotameric form): 12.53 (br s, 1H), 11.04/10.98 (br s. 1H), 8.29/7.88 (d, J = 2.0 Hz, 1H), 7.67 (s, 1H), 7.59-7.35 (m, 3H), 4.01-3.93 (m, 2H), 3.82-3.80 (m, 1H), 3.66-3.62 (m, 1H), 3.44-3.42 (m, 1H), 3.30-3.26 (m, 1H), 2.67-2.52 (m, 1H), 2.42-2.36 (m, 2H), 2.23-2.00 (m, 1H), 1.93-1.89 (m, 2H), 1.81-1.72 (m, 3H), 1.66-1.47 (m, 1H). |
| 149 | N-benzyl-3,5- dichloroaniline | 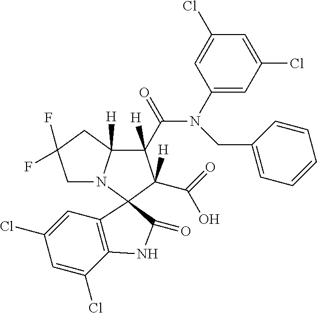 [SMILES: O=C(O)[C@H]1[C@@H](C(=O)N(Cc2ccccc2)c2cc(Cl)cc(Cl)c2)[C@H]2CC(F)(F)CN2[C@]12C(=O)Nc1c(Cl)cc(Cl)cc12] | 653.9 | | (500 MHz, DMSO-d₆) (Exist in rotameric form): 12.72 (s, 1H), 11.07/11.01 (s, 1H), 8.32 (d, J = 2.0 Hz, 1H), 7.58-7.24 (m, 9H), 5.44 (d, J = 15.5 Hz, 1H), 4.56 (d, J = 15.5 Hz, 1H), 4.14-4.04 (m, 1H), 3.91-3.82 (m, 1H), 3.56 (t, J = 7.0 Hz, 1H), 3.31 (m, 1H), 2.64-2.54 (m, 1H), 2.42-2.36 (m, 1H), 2.20-2.13 (m, 1H). |
| 150 | 2-((3,5- dichlorophenyl)amino) ethan-1-ol | 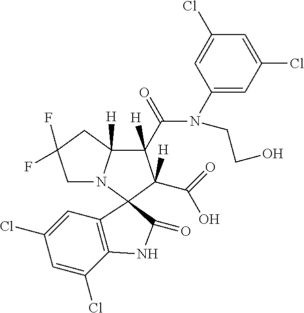 [SMILES: O=C(O)[C@H]1[C@@H](C(=O)N(CCO)c2cc(Cl)cc(Cl)c2)[C@H]2CC(F)(F)CN2[C@]12C(=O)Nc1c(Cl)cc(Cl)cc12] | 607.9 | | (500 MHz, DMSO-d₆): 12.71 (s, 1H), 11.12 (s, 1H), 7.54 (d, J = 4.5 Hz, 2H), 6.63 (s, 3H), 6.39-6.35 (m, 1H), 4.28-4.26 (m, 1H), 4.18-4.15 (m, 1H), 3.95-3.90 (m, 1H), 3.81 (d, J = 12.0 Hz, 1H), 3.74-3.69 (m, 1H), 3.36-3.34 (m, 2H), 2.87-2.76 (m, 2H), 2.68-2.61 (m, 1H), 2.50-2.36 (m, 1H). |
| 151 | 3,5-dichloro-N- isobutylaniline | 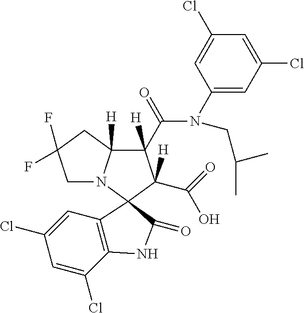 [SMILES: CC(C)CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] | 620 | | (500 MHz, DMSO-d₆) (Exist in rotameric form): 12.56 (br s, 1H), 11.04/10.98 (br s, 1H), 8.27/7.87 (s, 1H), 7.66-7.40 (m, 4H), 4.12-4.02 (m, 1H), 3.85-3.80 (m, 2H), 3.50-3.44 (m, 1H), 3.42-3.38 (m, 1H), 3.31-3.17 (m, 1H), 2.64-2.55 (m, 1H), 2.50-2.36 (m, 1H), 2.12-1.99 (m, 1H), 1.66-1.61 (m, 1H), 0.92-0.84 (m, 6H). |
| 152 | 3,5-dichloro-N- cyclobutylaniline | 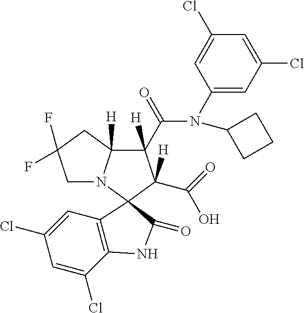 [SMILES: O=C(O)[C@H]1[C@@H](C(=O)N(c2cc(Cl)cc(Cl)c2)C2CCC2)[C@H]2CC(F)(F)CN2[C@]12C(=O)Nc1c(Cl)cc(Cl)cc12] | 617.9 | | (500 MHz, DMSO-d₆) (Exist in rotameric form): 12.55 (s, 1H), 11.03/10.97 (s, 1H), 8.28 (t, J = 2.5 Hz, 1H), 7.90-7.22 (m, 4H), 4.89/4.46 (m, 1H), 4.16-3.77 (m, 3H), 3.31-3.24 (m, 2H), 2.58-2.51 (m, 1H), 2.49-2.40 (m, 1H), 2.14-2.04 (m, 3H), 1.73-1.52 (m, 3H). |
| 153 | 2-((3,5-dichlorophenyl) amino)-N,N- dimethylacetamide | 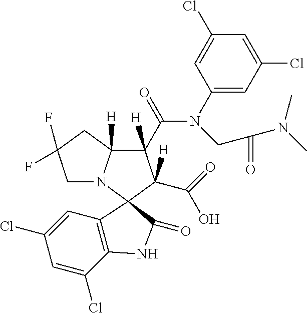 [SMILES: CN(C)C(=O)CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] | 648.9 | | (500 MHz, DMSO-d₆) (Exist in rotameric form): 12.51 (s, 1H), 10.99 (s, 1H), 8.20/7.89 (d, J = 2.5 Hz, 1H), 7.68/7.56 (d, J = 2.0 Hz, 1H), 7.45-7.27 (m, 3H), 5.03/4.99 (s, 1H), 4.18/4.14 (s, 1H), 4.03 (d, J = 9.5 Hz, 1H), 3.85-3.81 (m, 1H), 3.56 (t, J = 8.5 Hz, 1H), 3.32-3.20 (m, 1H), 2.97/2.95 (s, 3H), 2.89/2.87 (s, 3H), 2.71-2.69 (m, 1H), 2.56-2.50 (m, 1H), 2.32-2.26 (m, 1H). |
| 154 | 3,5-dichloro-N-(3,3,3- trifluoro-2,2- dimethylpropyl)aniline | 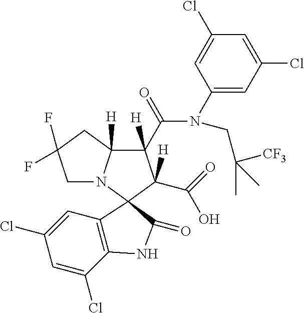 [SMILES: CC(C)(CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1)C(F)(F)F] | 688 | | (500 MHz, DMSO-d₆): 12.68 (br s, 1H), 11.01 (br s, 1H), 8.20 (d, J = 1.5 Hz, 1H), 7.75-7.74 (m, 1H), 7.65 (s, 1H), 7.62-7.42 (m, 2H), 4.35 (d, J = 14.5 Hz, 1H), 4.08 (d, J = 7.5 Hz, 1H), 3.83-3.75 (m, 2H), 3.62 (t, J = 7.0 Hz, 1H), 3.32-3.29 (m, 1H), 2.61-2.51 (m, 1H), 2.37-2.29 (m, 1H), 2.19-2.05 (m, 1H), 1.17 (s, 3H), 1.00 (s, 3H). |
| 155 | 3,5-dichloro-N-((1- (trifluoromethyl) cyclopropyl) methyl)aniline | 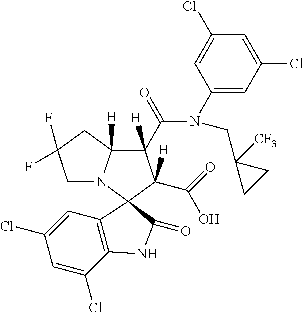 [SMILES: O=C(O)[C@H]1[C@@H](C(=O)N(CC2(C(F)(F)F)CC2)c2cc(Cl)cc(Cl)c2)[C@H]2CC(F)(F)CN2[C@]12C(=O)Nc1c(Cl)cc(Cl)cc12] | 686 | | (500 MHz, DMSO-d₆) (Exist in rotameric form): 12.60 (br s, 1H), 11.05/11.01 (s, 1H), 8.20 (d, J = 2.0 Hz, 1H), 7.75-7.65 (m, 1H), 7.49-7.36 (m, 3H), 4.29 (d, J = 15.5 Hz, 1H), 4.04-4.01 (m, 2H), 3.84-3.80 (m, 1H), 3.51 (t, J = 7.0 Hz, 1H), 3.29-3.23 (m, 1H), 2.58-2.52 (m, 1H), 2.36-2.34 (m, 1H), 2.09-2.07 (m, 1H), 0.92-0.84 (m, 2H), 0.76-0.68 (m, 2H). |
| 156 | N-(cyclopropylmethyl)- [1,1′-biphenyl]-3-amine | 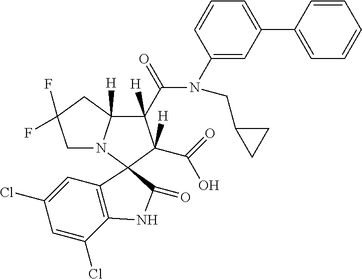 [SMILES: O=C(O)[C@H]1[C@@H](C(=O)N(CC2CC2)c2cccc(-c3ccccc3)c2)[C@H]2CC(F)(F)CN2[C@]12C(=O)Nc1c(Cl)cc(Cl)cc12] | | 624.1 | (500 MHz, DMSO-d₆) (Exist in rotameric form): 12.50 (br s, 1H), 10.97 (s, 1H), 8.36 (d, J = 1.5 Hz, 1H), 7.72-7.52 (m, 5H), 7.50-7.47 (m, 3H), 7.44-7.31 (m, 2H), 3.95 (d, J = 7.0 Hz, 1H), 3.81-3.60 (m, 1H), 3.69-3.68 (m, 1H), 3.64-3.61 (m, 1H), 3.50 (t, J = 7.0 Hz, 1H), 3.28-3.26 (m, 1H), 2.58-2.55 (m, 1H), 2.50-2.44 (m, 1H), 2.39-2.17 (m, 1H), 0.99-0.95 (m, 1H), 0.47-0.43 (m, 2H), 0.23-0.13 (m, 2H). |
| 157 | N-methyl-3- (trifluoromethyl) aniline | 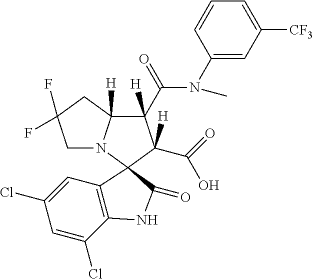 [SMILES: CN(C(=O)[C@H]1[C@H]2CC(F)(F)CN2[C@]2(C(=O)Nc3c(Cl)cc(Cl)cc32)[C@H]1C(=O)O)c1cccc(C(F)(F)F)c1] | 578 | | (500 MHz, DMSO-d₆) (Exist in rotameric form): 13.25-11.80 (br s. 1H), 11.20-10.80 (br s. 1H), 8.35 (d, J = 1.5 Hz, 1H), 7.92-7.61 (m, 4H), 7.44/7.42 (s, 1H), 4.15-4.09 (m, 1H), 3.89-3.87 (m, 1H), 3.78-3.74 (m, 1H), 3.43-3.38 (m, 1H), 3.27 (s, 3H), 2.57-2.50 (m, 1H), 2.37-2.29 (m, 1H), 2.17-2.10 (m, 1H). |
| 158 | 3-((3,5-dichlorophenyl) amino)cyclobutan-1-ol | 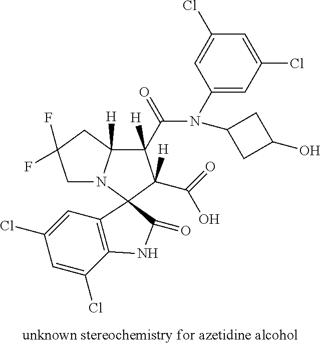 [SMILES: O=C(O)[C@H]1[C@@H](C(=O)N(c2cc(Cl)cc(Cl)c2)C2CC(O)C2)[C@H]2CC(F)(F)CN2[C@]12C(=O)Nc1c(Cl)cc(Cl)cc12] | 634 | | (500 MHz, DMSO-d₆): 12.80 (br s, 1H), 11.14 (s, 1H), 7.62 (s, 1H), 7.54 (d, J = 1.5 Hz, 1H), 6.65-6.62 (m, 2H), 6.49 (d, J = 1.5 Hz, 2H), 4.78 (m, 1H), 3.91-3.81 (m, 1H), 3.78-3.74 (m, 2H), 3.62-3.53 (m, 1H), 2.90-2.76 (m, 5H), 2.55-2.45 (m, 1H), 1.90-1.88 (m, 2H). |
| 159 | 3-((3,5-dichlorophenyl) amino)cyclobutan-1-ol | 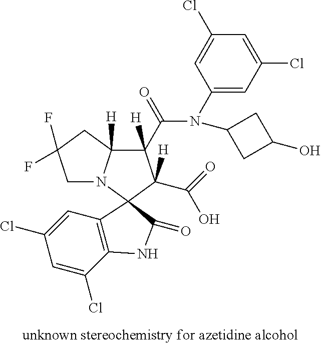 [SMILES: O=C(O)[C@H]1[C@@H](C(=O)N(c2cc(Cl)cc(Cl)c2)C2CC(O)C2)[C@H]2CC(F)(F)CN2[C@]12C(=O)Nc1c(Cl)cc(Cl)cc12] | 633.9 | | (500 MHz, DMSO-d₆): 12.80 (br s, 1H), 11.14 (s, 1H), 7.63 (br s, 1H), 7.55 (d, J = 1.5 Hz, 1H), 6.73 (d, J = 6.0 Hz, 1H), 6.65-6.63 (m, 1H), 6.44 (d, J = 2.0 Hz, 2H), 5.16-5.12 (m, 1H), 3.98-3.93 (m, 2H), 3.82-3.77 (m, 2H), 2.86-2.79 (m, 3H), 2.60-2.50 (m, 1H), 2.50-2.43 (m, 2H), 2.31-2.26 (m, 2H). |
| 160 | N1-(3,5- dichlorophenyl)-N2,N2- dimethylethane-1,2- diamine | 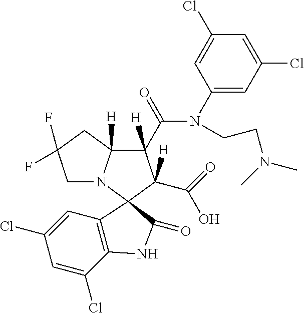 [SMILES: CN(C)CCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] | 634.9 | | (500 MHz, DMSO-d₆): 10.80 (s, 1H), 8.22 (d, J = 2.0 Hz, 1H), 7.70 (t, J = 2.0 Hz, 1H), 7.62 (br s, 2H), 7.41 (d, J = 2.0 Hz, 1H), 4.48 (br s, 1H), 3.90 (d, J = 8.0 Hz, 1H), 3.85-3.80 (m, 1H), 3.58-3.55 (m, 1H), 3.41-3.26 (m, 2H), 2.86-2.83 (m, 2H), 2.60 (br s, 6H), 2.60-2.50 (m, 1H), 2.30-2.22 (m, 2H). |
| 161 | N-methyl-[1,1′- biphenyl]-3-amine | 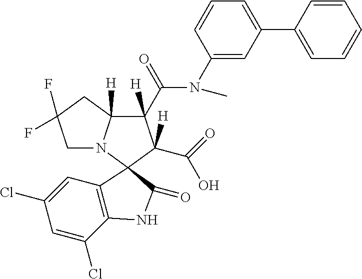 [SMILES: CN(C(=O)[C@H]1[C@H]2CC(F)(F)CN2[C@]2(C(=O)Nc3c(Cl)cc(Cl)cc32)[C@H]1C(=O)O)c1cccc(-c2ccccc2)c1] | 586 | | (500 MHz, DMSO-d₆): 8.40 (d, J = 2.0 Hz, 1H), 7.71-7.67 (m, 4H), 7.56 (t, J = 8.0 Hz, 1H), 7.50-7.45 (m, 3H), 7.41-7.38 (m, 2H), 3.92-3.91 (m, 1H), 3.81-3.76 (m, 1H), 3.56 (t, J = 6.5 Hz, 1H), 3.30 (s, 3H), 3.27-3.25 (m, 1H), 2.58-2.52 (m, 1H), 2.42-2.36 (m, 1H), 2.21-2.14 (m, 1H). |
| 162 | N-methyl-[1,1′- biphenyl]-2-amine | 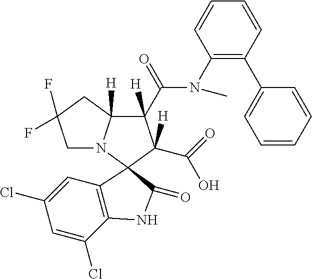 [SMILES: CN(C(=O)[C@H]1[C@H]2CC(F)(F)CN2[C@]2(C(=O)Nc3c(Cl)cc(Cl)cc32)[C@H]1C(=O)O)c1ccccc1-c1ccccc1] | 586 | | (500 MHz, DMSO-d₆) (Exist in rotameric form): 8.17 (d, J = 2.0 Hz, 1H), 7.55-7.35 (m, 10H), 3.69-3.67 (d, J = 8.0 Hz, 1H), 3.55-3.51 (m, 1H), 3.28 (s, 3H), 3.19-3.06 (m, 2H), 2.43-2.38 (m, 1H), 1.39-1.32 (m, 1H), 0.80-0.72 (m, 1H). |
| 163 | N-(cyclopropylmethyl)- 3- (trifluoromethyl)aniline | 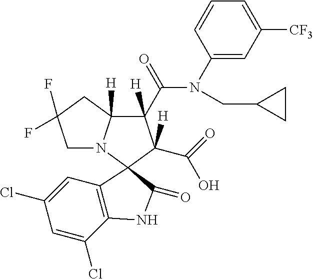 [SMILES: O=C(O)[C@H]1[C@@H](C(=O)N(CC2CC2)c2cccc(C(F)(F)F)c2)[C@H]2CC(F)(F)CN2[C@]12C(=O)Nc1c(Cl)cc(Cl)cc12] | 618 | | (500 MHz, DMSO-d₆) (Exist in rotameric form): 12.65 (br s, 1H), 11.00/10.98 (s, 1H), 8.31 (d, J = 1.5 Hz, 1H), 7.80-7.62 (m, 4H), 7.48-7.33 (m, 1H), 3.95-3.93 (m, 1H), 3.78-3.73 (m, 2H), 3.55-3.51 (m, 1H), 3.35-3.24 (m, 2H), 2.60-2.54 (m, 1H), 2.49-2.36 (m, 1H), 2.28-2.16 (m, 1H), 0.91-0.88 (m, 1H), 0.42-0.40 (m, 2H), 0.17-0.14 (m, 1H), 0.06-0.05 (m, 1H). |
| 164 | N-(cyclopropylmethyl)- 3-(2,2,2- trifluoroethyl)aniline | 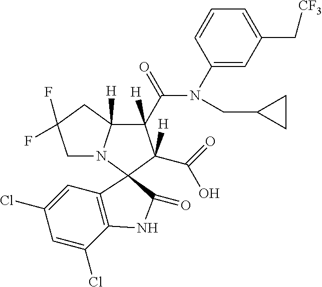 [SMILES: O=C(O)[C@H]1[C@@H](C(=O)N(CC2CC2)c2cccc(CC(F)(F)F)c2)[C@H]2CC(F)(F)CN2[C@]12C(=O)Nc1c(Cl)cc(Cl)cc12] | 632 | | (500 MHz, DMSO-d₆): 12.55 (br s, 1H), 10.98 (s, 1H), 7.33 (d, J = 2.0 Hz, 1H), 7.53-7.31 (m, 5H), 3.87 (d, J = 7.5 Hz, 1H), 3.76-3.71 (m, 3H), 3.59 (d, J = 6.0 Hz, 2H), 3.36-3.22 (m, 2H), 2.59-2.54 (m, 1H), 2.50-2.42 (m, 1H), 2.18 (m, 1H), 0.93-0.89 (m, 1H), 0.42-0.40 (m, 2H), 0.16-0.09 (m, 2H). |
| 165 | N-(cyclopropylmethyl)- 1,3-dimethyl-1H- pyrazol-4-amine | 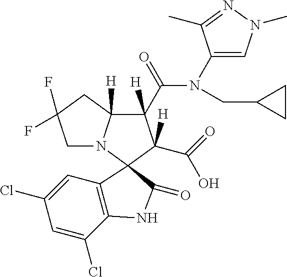 [SMILES: Cc1nn(C)cc1N(CC1CC1)C(=O)[C@H]1[C@H]2CC(F)(F)CN2[C@]2(C(=O)Nc3c(Cl)cc(Cl)cc32)[C@H]1C(=O)O] | | 566.1 | (500 MHz, DMSO-d₆) (exist in rotameric form): 12.57 (br s, 1H), 10.88 (br s, 1H), 7.87 (s, 1H), 7.54-7.51 (m, 1H), 6.67-6.61 (m, 1H), 4.10-3.72 (m, 6H), 3.53-3.32 (m, 2H), 2.98-2.63 (m, 1H), 2.63-2.50 (m, 1H), 2.48-2.25 (m, 1H), 2.22/2.09 (s, 3H), 1.63-1.46 (m, 1H), 0.93-0.77 (m, 1H), 0.39-0.28 (m, 2H), 0.12-0.09 (m, 2H). |
| 166 | N-(cyclopropylmethyl)- 1-methyl-3- (trifluoromethyl)-1H- pyrazol-4-amine | 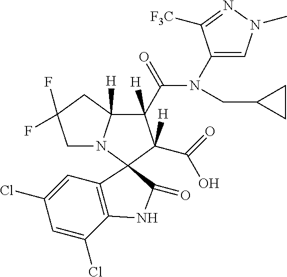 [SMILES: Cn1cc(N(CC2CC2)C(=O)[C@H]2[C@H]3CC(F)(F)CN3[C@]3(C(=O)Nc4c(Cl)cc(Cl)cc43)[C@H]2C(=O)O)c(C(F)(F)F)n1] | 622 | | (500 MHz, DMSO-d₆) (Exist in rotameric form): 12.68-12.13 (m, 1H), 11.14-10.87 (m, 1H), 8.19/8.15 (s, 1H), 7.93-7.42 (m, 2H), 4.02-3.91 (m, 6H), 3.46-3.36 (m, 1H), 3.15-2.75 (m, 2H), 2.64-2.59 (m, 1H), 2.30-2.10 (m, 2H), 0.93-0.85 (m, 1H), 0.44-0.40 (m, 2H), 0.22-0.09 (m, 2H). |
| 167 | N-(cyclopropylmethyl)- 1-phenyl-3- (trifluoromethyl)-1H- pyrazol-4-amine | 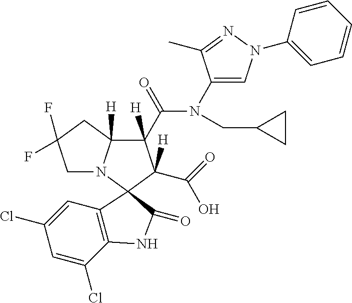 [SMILES: Cc1nn(-c2ccccc2)cc1N(CC1CC1)C(=O)[C@H]1[C@H]2CC(F)(F)CN2[C@]2(C(=O)Nc3c(Cl)cc(Cl)cc32)[C@H]1C(=O)O] | | 628.1 | (500 MHz, DMSO-d₆) (Exist in rotameric form): 8.75-8.67 (m, 1H), 7.86-7.67 (m, 2H), 7.52-7.43 (m, 3H), 7.37-7.27 (m, 1H), 6.74-6.65 (m, 1H), 4.14-3.02 (m, 5H), 2.79-2.49 (m, 3H), 2.36/2.24 (s, 3H), 1.81-1.54 (m, 1H), 0.98-0.89 (m, 1H), 0.46-0.36 (m, 2H), 0.12-0.11 (m, 2H). |
| 168 | 4-((cyclopropylmethyl) amino)-1-methyl-1H- pyrazole-3-carbonitrile | 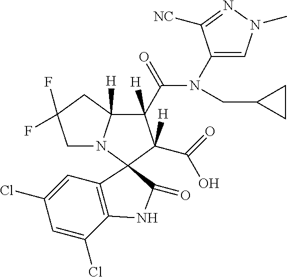 [SMILES: Cn1cc(N(CC2CC2)C(=O)[C@H]2[C@H]3CC(F)(F)CN3[C@]3(C(=O)Nc4c(Cl)cc(Cl)cc43)[C@H]2C(=O)O)c(C#N)n1] | 579 | | (500 MHz, DMSO-d₆) (Exist in rotameric form): 12.50 (br s, 1H), 10.99 (s, 1H), 8.21 (d, J = 2.0 Hz, 1H), 8.00 (s, 1H), 7.48 (d, J = 2.0 Hz, 1H), 4.20-3.88 (m, 2H), 3.96/3.95 (s, 3H), 3.66-3.62 (m, 1H), 3.54 (t, J = 7.0 Hz, 1H), 3.44-3.40 (m, 1H), 3.32-3.25 (m, 1H), 2.63-2.51 (m, 1H), 2.50-2.40 (m, 1H), 2.19-2.07 (m, 1H), 0.92-0.90 (m, 1H), 0.47-0.44 (m, 2H), 0.23-0.22 (m, 2H). |
| 169 | 3,5-dichloro-N- methylaniline | 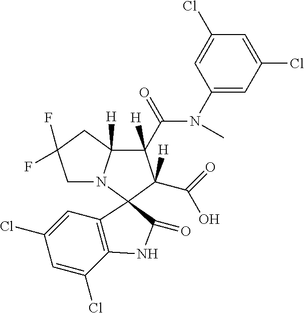 [SMILES: CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] | 578 | | (500 MHz, DMSO-d₆) (Exist in rotameric form) 6: 12.54 (br s, 1H), 11.07/11.00 (s, 1H), 8.30/7.88 (d, J = 1.5 Hz, 1H), 7.66 (s, 1H), 7.56-7.43 (m, 3H), 4.17-4.10 (m, 1H), 4.01 (d, J = 7.5 Hz, 1H), 3.84-3.80 (m, 1H), 3.54 (t, J = 7.0 Hz, 1H), 3.39/3.24 (s, 3H), 2.67-2.50 (m, 1H), 2.40-2.32 (m, 1H), 2.14-2.06 (m, 1H) |
| 170 | 2-(3-(tert-butyl)phenyl)- N,2-dimethylpropan-1- amine | 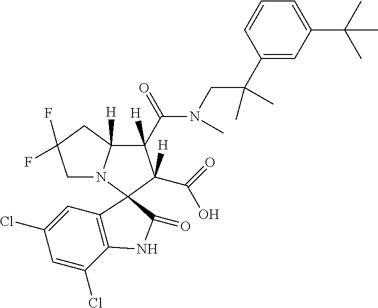 [SMILES: CN(CC(C)(C)c1cccc(C(C)(C)C)c1)C(=O)[C@H]1[C@H]2CC(F)(F)CN2[C@]2(C(=O)Nc3c(Cl)cc(Cl)cc32)[C@H]1C(=O)O] | 622.2 | | (500 MHz, DMSO-d₆) (Exist in rotameric form): 12.62-12.01 (br s. 1H), 11.27-10.80 (br s. 1H), 8.14-8.11 (m, 1H), 7.51-7.42 (m, 2H), 7.25-7.23 (m, 3H), 4.03-3.93 (m, 2H), 3.85-3.61 (m, 3H), 3.20-3.06 (m, 1H), 2.64-2.44 (m, 4H), 2.37-2.07 (m, 1H), 1.95-1.88 (m, 1H), 1.41-1.28 (m, 15H). |
| 171 | 3,5-dichloro-N- ethylaniline | 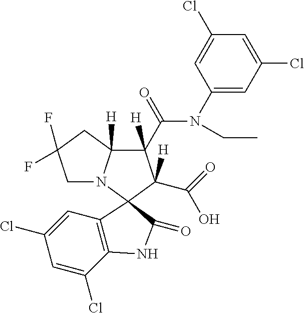 [SMILES: CCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] | 592.1 | | (500 MHz, DMSO-d₆) (Exist in rotameric form): 12.72 (br s, 1H), 11.10/10.99 (s, 1H), 8.30/7.92 (d, J = 1.5 Hz, 1H), 7.68-7.35 (m, 4H), 4.15-3.79 (m, 3H), 3.57-3.53 (m, 1H), 3.48-3.41 (m, 1H), 3.28-3.26 (m, 1H), 2.60-2.50 (m, 1H), 2.41-2.35 (m, 1H), 2.16-2.07 (m, 1H), 1.15-1.04 (m, 3H). |
| 172 | 3,5-dichloro-N- propylaniline | 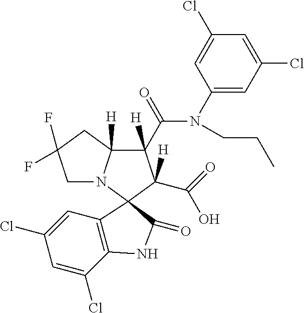 [SMILES: CCCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] | | 604 | (500 MHz, DMSO-d₆) (Exist in rotameric form): 12.60 (br s, 1H), 11.10/10.99 (s, 1H), 10.20/8.28 (d, J = 2.0 Hz, 1H), 7.90-7.37 (m, 4H), 4.13-4.00 (m, 1H), 3.86-3.70 (m, 2H), 3.52-3.45 (m, 2H), 3.32-3.25 (m, 1H), 2.60-2.57 (m, 1H), 2.50-2.36 (m, 1H), 2.20-2.00 (m, 1H), 1.48-1.42 (m, 2H), 0.89-0.86 (m, 3H). |
| 173 | 3,5-dichloro-N- (cyclopropylmethyl) aniline | 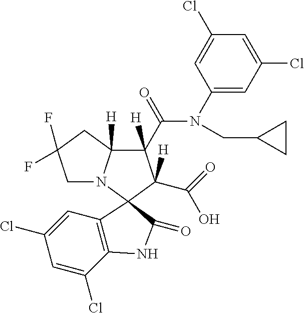 [SMILES: O=C(O)[C@H]1[C@@H](C(=O)N(CC2CC2)c2cc(Cl)cc(Cl)c2)[C@H]2CC(F)(F)CN2[C@]12C(=O)Nc1c(Cl)cc(Cl)cc12] | | 616 | (500 MHz, DMSO-d₆) (Exist in rotameric form): 12.50 (br s, 1H), 10.99 (s, 1H), 8.28/7.91 (d, J = 1.5 Hz, 1H), 7.69 (s, 1H), 7.64-7.34 (m, 3H), 4.02-4.00 (d, J = 7.5 Hz, 1H), 3.85-3.76 (m, 2H), 3.45-3.41 (m, 2H), 3.32-3.26 (m, 1H), 2.60-2.55 (m, 1H), 2.41-2.36 (m, 1H), 2.25-2.10 (m, 1H), 0.90-0.80 (m, 1H), 0.42-0.40 (m, 2H), 0.19-0.17 (m, 1H), 0.08-0.07 (m, 1H). |
| 174 | 3,5-dichloro-N- isopropylaniline | 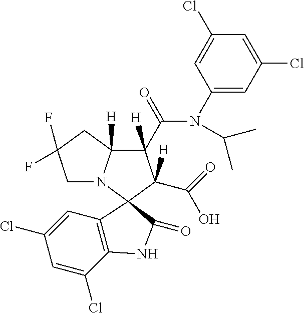 [SMILES: CC(C)N(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] | | 606 | (500 MHz, DMSO-d₆) (Exist in rotameric form): 12.52 (br s, 1H), 11.05/10.97 (s, 1H), 8.31 (d, J = 2.0 Hz, 1H), 7.75 (d, J = 2.0 Hz, 1H), 7.48 (d, J = 2.0 Hz, 1H), 7.43-7.37 (m, 1H), 7.24-7.22 (m, 1H), 4.93-4.91 (m, 1H), 3.96-3.95 (m, 1H), 3.81-3.77 (m, 1H), 3.32-3.23 (m, 2H), 2.58-2.50 (m, 1H), 2.45-2.41 (m, 1H), 2.10-2.07 (m, 1H), 1.05-1.02 (m, 6H). |
| 175 | 3,5-dichloro-N- cyclopentylaniline | 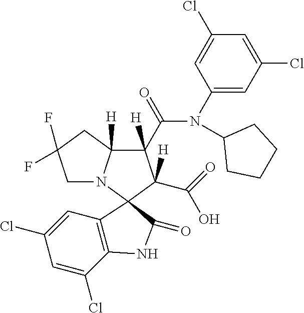 [SMILES: O=C(O)[C@H]1[C@@H](C(=O)N(c2cc(Cl)cc(Cl)c2)C2CCCC2)[C@H]2CC(F)(F)CN2[C@]12C(=O)Nc1c(Cl)cc(Cl)cc12] | 632 | | (500 MHz, DMSO-d₆) (exist in rotameric form): 12.55 (br s, 1H), 10.94 (s, 1H), 8.30 (d, J = 1.5 Hz, 1H), 7.73-7.22 (m, 4H), 4.80-4.76 (m, 1H), 3.96 (d, J = 7.5 Hz, 1H), 3.82-3.78 (m, 1H), 3.29-3.22 (m, 2H), 2.60-2.50 (m, 1H), 2.49-2.38 (m, 1H), 2.14-2.07 (m, 1H), 1.89-1.82 (m, 2H), 1.51-1.50 (m, 4H), 1.30-1.21 (m, 2H). |
| 176 | N-methyl-2- (trifluoromethyl)aniline | 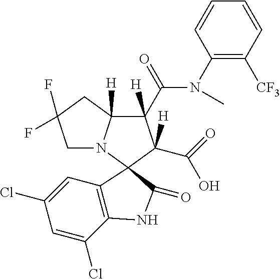 [SMILES: CN(C(=O)[C@H]1[C@H]2CC(F)(F)CN2[C@]2(C(=O)Nc3c(Cl)cc(Cl)cc32)[C@H]1C(=O)O)c1ccccc1C(F)(F)F] | 578.1 | | (500 MHz, DMSO-d₆) (Exist in rotameric form): 12.52 (br s, 1H), 11.04/10.99 (s, 1H), 8.20/8.05 (d, J = 2.0 Hz, 1H), 7.89-7.78 (m, 2H), 7.69-7.39 (m, 3H), 4.22-4.15 (m, 1H), 3.94-3.90 (m, 1H), 3.37-3.28 (m, 1H), 3.19-3.13 (m, 1H), 3.14 (s, 3H), 2.63-2.57 (m, 1H), 2.50-2.29 (m, 1H), 2.28-2.07 (m, 1H). |
| 177 | 3,5-dichloro-N- (cyclohexylmethyl) aniline | 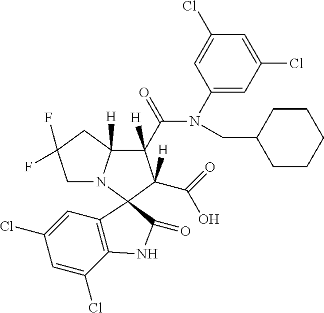 [SMILES: O=C(O)[C@H]1[C@@H](C(=O)N(CC2CCCCC2)c2cc(Cl)cc(Cl)c2)[C@H]2CC(F)(F)CN2[C@]12C(=O)Nc1c(Cl)cc(Cl)cc12] | 660 | | (500 MHz, DMSO-d₆) (Exist in rotameric form): 12.57 (br s, 1H), 11.04/10.98 (s, 1H), 8.27 (d, J = 1.5 Hz, 1H), 7.87-6.87 (m, 4H), 4.01 (d, J = 7.5 Hz, 1H), 3.92-3.89 (m, 1H), 3.82-3.80 (m, 1H), 3.49 (t, J = 7.0 Hz, 1H), 3.40-3.37 (m, 1H), 3.30-3.26 (m, 1H), 2.59-2.51 (m, 1H), 2.39-2.37 (m, 1H), 2.18-2.08 (m, 1H), 1.84-1.81 (m, 1H), 1.64-1.58 (m, 4H), 1.40-1.30 (m, 1H), 1.16-1.09 (m, 3H), 0.98-0.96 (m, 2H). |
| 178 | 3,5-dichloro-N- (cycloheptylmethyl) aniline | 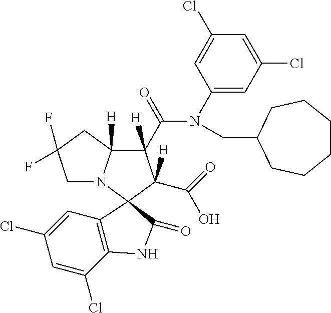 [SMILES: O=C(O)[C@H]1[C@@H](C(=O)N(CC2CCCCCC2)c2cc(Cl)cc(Cl)c2)[C@H]2CC(F)(F)CN2[C@]12C(=O)Nc1c(Cl)cc(Cl)cc12] | 674 | | (500 MHz, DMSO-d₆) (Exist in rotameric form): 12.57 (br s, 1H), 11.04/10.98 (s, 1H), 8.27/7.86 (d, J = 2.0 Hz, 1H), 7.67-7.38 (m, 4H), 4.02 (d, J = 7.5 Hz, 1H), 3.95-3.91 (m, 1H), 3.82-3.80 (m, 1H), 3.49 (t, J = 7.5 Hz, 1H), 3.39-3.36 (m, 1H), 3.31-3.25 (m, 1H), 2.54-2.49 (m, 1H), 2.37-2.36 (m, 1H), 2.25-2.05 (m, 1H), 1.75-1.70 (m, 1H), 1.63-1.59 (m, 3H), 1.46-1.42 (m, 5H), 1.36-1.15 (m, 4H). |
| 179 | 3,5-dichloro-4-methoxy- N-methylaniline | 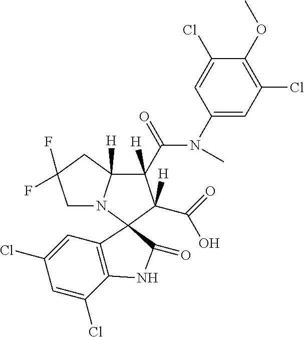 [SMILES: COc1c(Cl)cc(N(C)C(=O)[C@H]2[C@H]3CC(F)(F)CN3[C@]3(C(=O)Nc4c(Cl)cc(Cl)cc43)[C@H]2C(=O)O)cc1Cl] | 608.1 | | (500 MHz, DMSO-d₆) (Exist in rotameric form): 12.50 (br s, 1H), 11.10/10.99 (s, 1H), 8.31/7.88 (d, J = 1.5 Hz, 1H), 7.57-7.35 (m, 3H), 4.00 (d, J = 7.5 Hz, 1H), 3.86 (s, 3H), 3.85-3.80 (m, 1H), 3.56-3.53 (m, 1H), 3.37/3.23 (s, 3H), 3.30-3.24 (m, 1H), 2.60-2.55 (m, 1H), 2.42-2.36 (m, 1H), 2.18-2.05 (m, 1H). |
| 180 | N-(cyclopropylmethyl)- [1,1′-biphenyl]-2-amine | 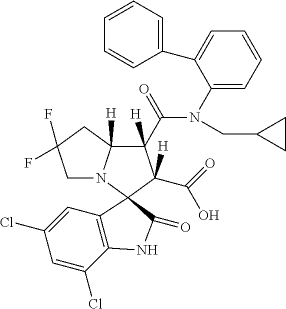 [SMILES: O=C(O)[C@H]1[C@@H](C(=O)N(CC2CC2)c2ccccc2-c2ccccc2)[C@H]2CC(F)(F)CN2[C@]12C(=O)Nc1c(Cl)cc(Cl)cc12] | 626 | | (500 MHz, DMSO-d₆) (Exist in rotameric form): 12.46 (br s, 1H), 11.10/10.92 (br s, 1H), 8.16-8.02 (m, 1H), 7.68-7.33 (m, 10H), 4.23-4.19 (m, 1H), 3.78 (d, J = 8.5 Hz, 1H), 3.64-3.63 (m, 1H), 3.18-3.13 (m, 2H), 2.89-2.86 (m, 1H), 2.50-2.43 (m, 1H), 1.50-1.35 (m, 1H), 1.28-1.20 (m, 1H), 1.02-0.80 (m, 1H), 0.52-0.48 (m, 2H), 0.35-0.32 (m, 1H), 0.18-0.16 (m, 1H). |
| 181 | N-(cyclopentylmethyl)- 3- (trifluoromethyl)aniline | 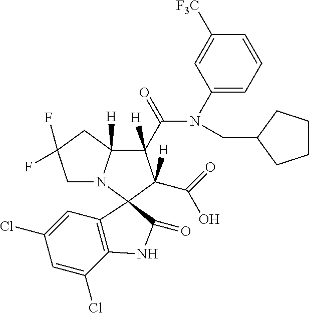 [SMILES: O=C(O)[C@H]1[C@@H](C(=O)N(CC2CCCC2)c2cccc(C(F)(F)F)c2)[C@H]2CC(F)(F)CN2[C@]12C(=O)Nc1c(Cl)cc(Cl)cc12] | 646 | | (500 MHz, DMSO-d₆) (Exist in rotameric form): 12.60 (br s, 1H), 11.00/10.98 (s, 1H), 8.31/7.90 (d, J = 2.0 Hz, 1H), 7.78-7.55 (m, 4H), 7.48/7.43 (d, J = 1.5 Hz, 1H), 3.95-3.94 (m, 2H), 3.77-3.75 (m, 1H), 3.62-3.55 (m, 1H), 3.38-3.25 (m, 2H), 2.57-2.50 (m, 1H), 2.43-2.31 (m, 1H), 2.25-2.07 (m, 1H), 1.92-1.89 (m, 1H), 1.66-1.58 (m, 4H), 1.47-1.45 (m, 2H), 1.37-1.35 (m, 1H), 1.17-1.14 (m, 1H). |
| 182 | 3,5-dichloro-N-(3,3- dimethylbutyl)aniline | 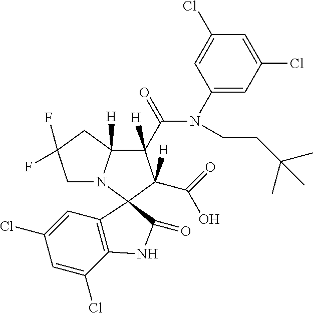 [SMILES: CC(C)(C)CCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] | 648 | | ¹H NMR (500 MHz, DMSO-d₆) (Exist in rotameric form): 12.54/12.50 (br s, 1H), 11.05/10.99 (s, 1H), 8.30/7.92 (d, J = 2.0 Hz, 1H), 7.68-7.40 (m, 4H), 4.00-3.95 (m, 2H), 3.81-3.79 (m, 1H), 3.48-3.42 (m, 2H), 3.36-3.25 (m, 1H), 2.64-2.55 (m, 1H), 2.50-2.34 (m, 1H), 2.20-2.05 (m, 1H), 1.49-1.47 (m, 1H), 1.36-1.32 (m, 1H), 0.88/0.87 (s, 9H). |
| 183 | N-(cyclopropylmethyl)- 3-(perfluoroethyl)aniline | 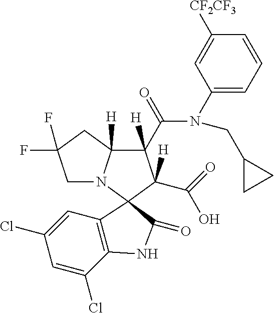 | | 666 | ¹H NMR(500 MHz, DMSO-d₆) (Exist in rotameric form): 12.55 (br s, 1H), 11.01/10.99 (s, 1H), 8.30/7.95 (d, J = 2.0 Hz, 1H), 7.79-7.73 (m, 2H), 7.70-7.61 (m, 2H), 7.49/7.43 (d, J = 2.0 Hz, 1H), 3.91 (d, J = 7.5 Hz, 1H), 3.79-3.76 (m, 2H), 3.53-3.45 (m, 1H), 3.32-3.24 (m, 2H), 2.61-2.55 (m, 1H), 2.45-2.30 (m, 1H), 2.29-2.15 (m, 1H), 0.90-0.87 (m, 1H), 0.41-0.38 (m, 2H), 0.15-0.12 (m, 1H), 0.03-0.01 (m, 1H). |
| 184 | N-(cyclopropylmethyl)- 3-(1,1,1-trifluoro-2- methylpropan-2- yl)aniline | 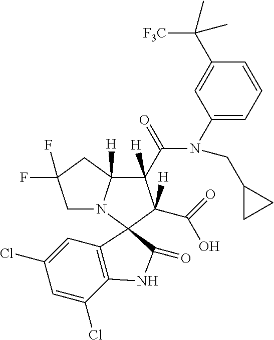 [SMILES: CC(C)(c1cccc(N(CC2CC2)C(=O)[C@H]2[C@H]3CC(F)(F)CN3[C@]3(C(=O)Nc4c(Cl)cc(Cl)cc43)[C@H]2C(=O)O)c1)C(F)(F)F] | 660.1 | | ¹H NMR(500 MHz, DMSO-d₆) (Exist in rotameric form): 12.52 (br s, 1H), 10.99 (br s, 1H), 8.33 (d, J = 2.0 Hz, 1H), 7.59-7.48 (m, 2H), 7.33/6.65 (d, J = 7.5 Hz, 1H), 7.12/6.58 (m, 1H), 5.85/5.15 (m, 1H), 3.91-3.86 (m, 2H), 3.75-3.42 (m, 3H), 3.32-3.25 (m, 1H), 2.58-2.56 (m, 1H), 2.44-2.32 (m, 1H), 2.31-2.18 (m, 1H), 1.58 (s, 3H), 1.56 (s, 3H), 0.93-0.91 (m, 1H), 0.43-0.39 (m, 2H), 0.20-0.15 (m, 1H), 0.07-0.02 (m, 1H). |
| 185 | 3,5-dichloro-N-(2,2,3,3- tetramethylbutyl)aniline | 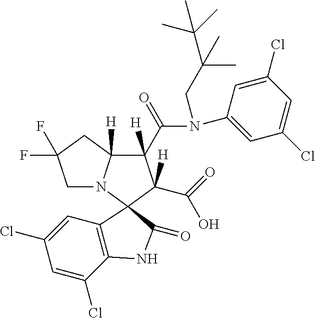 [SMILES: CC(C)(C)C(C)(C)CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] | 676 | | ¹H NMR(500 MHz, DMSO-d₆) (Exist in rotameric form): 12.58 (br s, 1H), 11.05/10.99 (s, 1H), 8.28 (d, J = 2.0 Hz, 1H), 7.68-7.44 (m, 4H), 4.25-4.17 (m, 1H), 4.03 (d, J = 7.5 Hz, 1H), 3.81-3.77 (m, 1H), 3.66-3.62 (m, 2H), 3.29-3.25 (m, 1H), 2.60-2.55 (m, 1H), 2.36-2.31 (m, 1H), 2.12-2.07 (m, 1H), 0.89 (s, 9H), 0.87 (s, 3H), 0.64 (s, 3H). |
| 186 | 3,5-dichloro-N- ((2,2,3,3-tetramethyl- cyclopropyl) methyl)aniline | 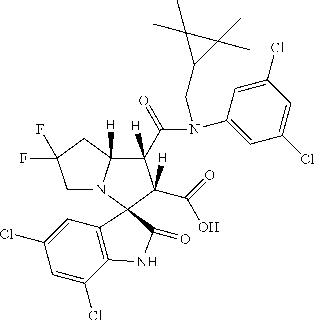 [SMILES: CC1(C)C(CN(C(=O)[C@H]2[C@H]3CC(F)(F)CN3[C@]3(C(=O)Nc4c(Cl)cc(Cl)cc43)[C@H]2C(=O)O)c2cc(Cl)cc(Cl)c2)C1(C)C] | 674 | | ¹H NMR(500 MHz, DMSO-d₆) (Exist in rotameric form): 12.55 (br s, 1H), 11.05/10.99 (s, 1H), 8.30/7.91 (d, J = 1.5 Hz, 1H), 7.71 (t, J = 2.0 Hz, 1H), 7.49 (d, J = 2.0 Hz, 1H), 7.44-7.33 (m, 2H), 4.13-4.09 (m, 1H), 4.01 (d, J = 7.5 Hz, 1H), 3.84-3.80 (m, 1H), 3.48-3.42 (m, 2H), 3.30-3.26 (m, 1871871H), 2.62-2.57 (m, 1H), 2.49-2.36 (m, 1H), 2.20-2.05 (m, 1H), 0.98 (s, 3H), 0.92 (s, 3H), 0.77 (s, 3H), 0.54 (s, 3H), 0.32-0.30 (m, 1H). |
| 187 | 3,5-dichloro-N- (spiro[2.2]pentan-1- ylmethyl)aniline | 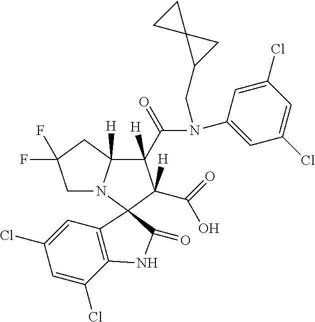 [SMILES: O=C(O)[C@H]1[C@@H](C(=O)N(CC2CC23CC3)c2cc(Cl)cc(Cl)c2)[C@H]2CC(F)(F)CN2[C@]12C(=O)Nc1c(Cl)cc(Cl)cc12] | 644 | | ¹H NMR(500 MHz, DMSO-d₆) (Exist in rotameric form): 12.60 (br s, 1H), 11.00/10.99 (s, 1H), 8.30 (dd, J = 8.5 Hz, J = 2.0 Hz, 1H), 7.71-7.69 (m, 1H), 7.49 (s, 1H), 7.44-7.34 (m, 2H), 4.36-4.12 (m, 1H), 4.02-4.00 (m, 1H), 3.83-3.74 (m, 2H), 3.49-3.40 (m, 1H), 3.30-3.15 (m, 1H), 2.66-2.50 (m, 1H), 2.48-2.35 (m, 1H), 2.20-2.06 (m, 1H), 1.32-1.15 (m, 1H), 0.93-0.89 (m, 1H), 0.69-0.49 (m, 5H). |
| 188 | 3,5-dichloro-N-((3- fluorobicyclo [1.1.1]pentan-1- yl)methyl)aniline | 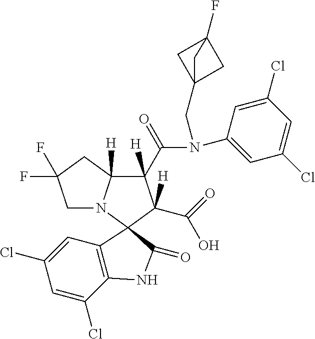 [SMILES: O=C(O)[C@H]1[C@@H](C(=O)N(CC23CC(F)(C2)C3)c2cc(Cl)cc(Cl)c2)[C@H]2CC(F)(F)CN2[C@]12C(=O)Nc1c(Cl)cc(Cl)cc12] | 662 | | ¹H NMR(500 MHz, DMSO-d₆) (Exist in rotameric form): 12.67/12.55 (br s, 1H), 11.06/11.00 (br s, 1H), 8.28/7.85 (d, J = 2.0 Hz, 1H), 7.66/7.58 (t, J = 1.5 Hz, 1H), 7.53-7.45 (m, 3H), 4.42 (d, J = 14.5 Hz, 1H), 4.07 (d, J = 7.5 Hz, 1H), 3.85-3.83 (m, 1H), 3.72 (d, J = 14.5 Hz, 1H), 3.50-3.47 (m, 1H), 3.37-3.32 (m, 1H), 2.60-2.51 (m, 1H), 2.40-2.36 (m, 1H), 2.18-2.03 (m, 1H), 2.02-1.90 (m, 6H). |
| 189 | N-(cyclopropylmethyl)- 3-(1-(trifluoromethyl) cyclopropyl)aniline | 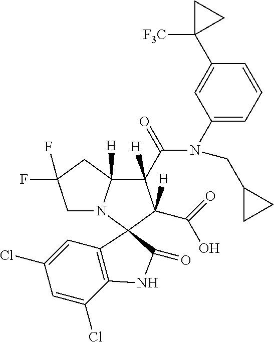 [SMILES: O=C(O)[C@H]1[C@@H](C(=O)N(CC2CC2)c2cccc(C3(C(F)(F)F)CC3)c2)[C@H]2CC(F)(F)CN2[C@]12C(=O)Nc1c(Cl)cc(Cl)cc12] | 658.1 | | ¹H NMR (400 MHz, DMSO-d₆) (Exist in rotameric form): 12.50 (br s, 1H), 11.00/10.99 (s, 1H), 8.31 (d, J = 2.0 Hz, 1H), 7.54-7.32 (m, 4H), 3.91-3.88 (m, 1H), 3.76-3.65 (m, 2H), 3.58-3.45 (m, 1H), 3.40-3.22 (m, 2H), 2.61-2.50 (m, 1H), 2.42-2.17 (m, 2H), 1.39-1.33 (m, 2H), 1.22-1.12 (m, 2H), 0.94-0.88 (m, 1H), 0.43-0.38 (m, 2H), 0.17-0.07 (m, 2H). |
| 190 | 1-((3r,5r,7r)-adamantan- 1-yl)-N- methylmethanamine | 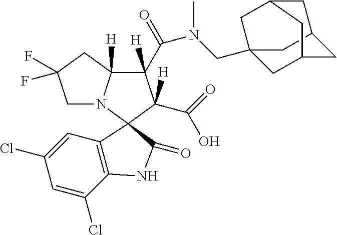 [SMILES: CN(CC12CC3CC(CC(C3)C1)C2)C(=O)[C@H]1[C@H]2CC(F)(F)CN2[C@]2(C(=O)Nc3c(Cl)cc(Cl)cc32)[C@H]1C(=O)O] | 582.1 | | ¹H NMR (400 MHz, DMSO-d₆) (Exist in rotameric form): 12.22 (br s, 1H), 10.99 (br s, 1H), 8.21/8.09 (d, J = 2.0 Hz, 1H), 7.44-7.43 (m, 1H), 4.09-3.96 (m, 3H), 3.23-3.00 (m, 3H), 3.06/2.92 (s, 3H), 2.62-2.54 (m, 1H), 2.50-2.43 (m, 1H), 2.11-2.00 (m, 1H), 1.96-1.90 (m, 3H), 1.75-1.50 (m, 12H). |
| 191 | 3,5-dichloro-N- hexylaniline | 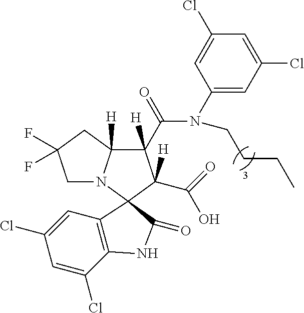 | 647.9 | | ¹H NMR (400 MHz, DMSO-d₆) (Exist in rotameric form): 12.55 (br s, 1H), 11.05/10.98 (s, 1H), 8.29/7.90 (d, J = 2.0 Hz, 1H), 7.68/7.59 (t, J = 2.0 Hz, 1H), 7.48 (d, J = 2.0 Hz, 1H), 7.44-7.38 (m, 2H), 4.14/4.01 (d, J = 7.6 Hz, 1H), 3.95-3.88 (m, 1H), 3.84-3.79 (m, 1H), 3.54-3.44 (m, 2H), 3.32-3.28 (m, 1H), 2.61-2.50 (m, 1H), 2.44-2.34 (m, 1H), 2.15-2.07 (m, 1H), 1.47-1.36 (m, 2H), 1.28-1.22 (m, 6H), 0.87-0.82 (m, 3H). |
| 192 | 3,5-dichloro-N- octylaniline | 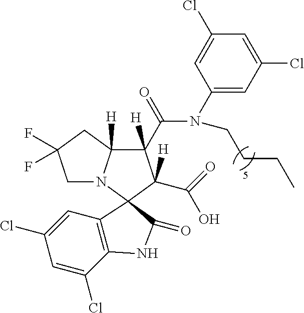 | 676.1 | | ¹H NMR (400 MHz, DMSO-d₆) (Exist in rotameric form): 12.53 (br s, 1H), 11.05/10.98 (s, 1H), 8.29/7.90 (d, J = 2.0 Hz, 1H), 7.68/7.59 (t, J = 2.0 Hz, 1H), 7.48 (d, J = 2.0 Hz, 1H), 7.44-7.38 (m, 2H), 4.14/4.00 (d, J = 7.2 Hz, 1H), 3.92-3.79 (m, 2H), 3.56-3.44 (m, 2H), 3.32-3.23 (m, 1H), 2.68-2.50 (m, 1H), 2.43-2.32 (m, 1H), 2.15-2.07 (m, 1H), 1.46-1.18 (m, 12H), 0.86-0.83 (m, 3H). |
| 193 | 3,5-dichloro-N- dodecylaniline | 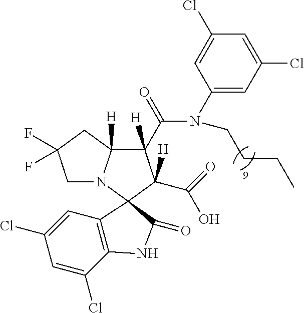 | | 730.2 | ¹H NMR(500 MHz, DMSO-d₆) (Exist in rotameric form): 12.55 (br s, 1H), 11.02/10.99 (s, 1H), 8.29/7.90 (d, J = 2.0 Hz, 1H), 7.68/7.59 (br s, 1H), 7.49-7.38 (m, 3H), 4.12/4.01 (d, J = 7.5 Hz, 1H), 3.89-3.79 (m, 2H), 3.55-3.37 (m, 3H), 2.64-2.50 (m, 1H), 2.42-2.35 (m, 1H), 2.14-2.08 (m, 1H), 1.50-1.23 (m, 20H), 0.88-0.83 (m, 3H). |
| 194 | N-(((3r,5r,7r)- adamantan-1- yl)methyl)-3,5- dichloroaniline | 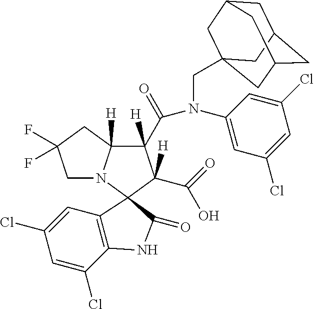 [SMILES: O=C(O)[C@H]1[C@@H](C(=O)N(CC23CC4CC(CC(C4)C2)C3)c2cc(Cl)cc(Cl)c2)[C@H]2CC(F)(F)CN2[C@]12C(=O)Nc1c(Cl)cc(Cl)cc12] | 712.1 | | ¹H NMR (400 MHz, DMSO-d₆) (Exist in rotameric form): 12.63 (br s, 1H), 11.02/10.99 (s, 1H), 8.25/7.81 (d, J = 2.0 Hz, 1H), 7.59-7.43 (m, 4H), 4.05 (d, J = 7.6 Hz, 1H), 3.88 (d, J = 14.4 Hz, 1H), 3.84-3.78 (m, 1H), 3.60 (t, J = 6.8 Hz, 1H), 3.37-3.27 (m, 2H), 2.60-2.50 (m, 1H), 2.38-2.30 (m, 1H), 2.13-2.04 (m, 1H), 1.90-1.82 (m, 3H), 1.63-1.47 (m, 6H), 1.50-1.40 (m, 6H). |
| 195 | N-(((1R,3S,5r,7r)- adamantan-2- yl)methyl)-3,5- dichloroaniline | 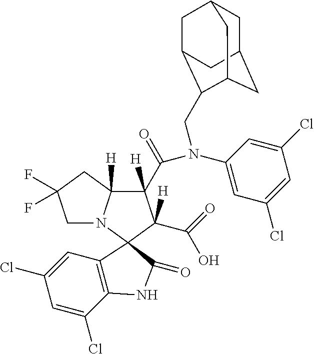 [SMILES: O=C(O)[C@H]1[C@@H](C(=O)N(CC2C3CC4CC(C3)CC2C4)c2cc(Cl)cc(Cl)c2)[C@H]2CC(F)(F)CN2[C@]12C(=O)Nc1c(Cl)cc(Cl)cc12] | 712.0 | | ¹H NMR (400 MHz, DMSO-d₆) (Exist in rotameric form): 12.60 (s, 1H), 11.05/10.98 (s, 1H), 8.30/7.91 (d, J = 1.6 Hz, 1H), 7.66/7.56 (br s, 1H), 7.48-7.42 (m, 3H), 4.42-4.37 (m, 1H), 4.02 (d, J = 7.6 Hz, 1H), 3.86-3.80 (m, 1H), 3.53-3.48 (m, 1H), 3.43-3.45 (m, 2H), 2.60-2.50 (m, 1H), 2.36-2.33 (m, 1H), 2.17-2.14 (m, 1H), 1.99-1.93 (m, 1H), 1.88-1.51 (m, 12H), 1.50-1.45 (m, 2H). |
| 196 | 3-chloro-N-(2,2,3,3- tetramethylbutyl)aniline | 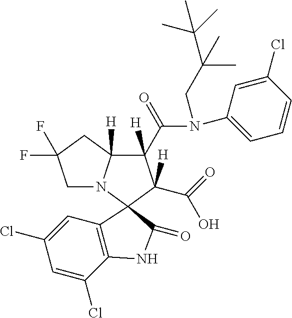 [SMILES: CC(C)(C)C(C)(C)CN(C(=O)[C@H]1[C@H]2CC(F)(F)CN2[C@]2(C(=O)Nc3c(Cl)cc(Cl)cc32)[C@H]1C(=O)O)c1cccc(Cl)c1] | 642.1 | | ¹H NMR (400 MHz, DMSO-d₆) (Exist in rotameric form): 12.51 (br s, 1H), 10.97 (br s, 1H), 8.30/7.82 (d, J = 1.6 Hz, 1H), 7.51-7.35 (m, 5H), 4.20-4.00 (m, 1H), 4.04-3.95 (m, 1H), 3.78-3.73 (m, 2H), 3.63-3.53 (m, 1H), 3.26-3.22 (m, 1H), 2.60-2.50 (m, 1H), 2.45-2.28 (m, 1H), 2.21-2.07 (m, 1H), 0.89 (s, 9H), 0.84 (s, 3H), 0.66 (s, 3H). |
| 197 | N-(2,2,3,3-tetramethyl- butyl)aniline | 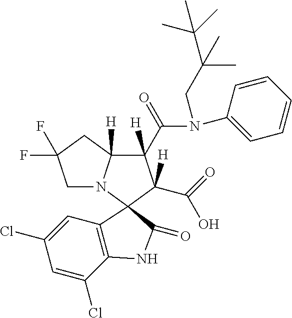 [SMILES: CC(C)(C)C(C)(C)CN(C(=O)[C@H]1[C@H]2CC(F)(F)CN2[C@]2(C(=O)Nc3c(Cl)cc(Cl)cc32)[C@H]1C(=O)O)c1ccccc1] | 608.1 | | ¹H NMR (400 MHz, DMSO-d₆): 12.41 (br s, 1H), 10.96 (br s, 1H), 8.34 (br s, 1H), 7.46-7.33 (m, 6H), 4.09-4.01 (m, 1H), 3.87-3.69 (m, 3H), 3.61-3.54 (m, 1H), 3.27-3.17 (m, 1H), 2.61-2.50 (m, 1H), 2.42-2.31 (m, 1H), 2.24-2.07 (m, 1H), 0.88 (s, 9H), 0.82 (s, 3H), 0.68 (s, 3H). |
| 198 | 3,5-dimethyl-N-(2,2,3,3- tetramethylbutyl)aniline | 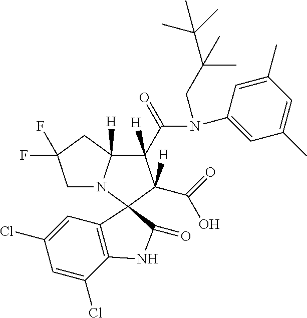 [SMILES: Cc1cc(C)cc(N(CC(C)(C)C(C)(C)C)C(=O)[C@H]2[C@H]3CC(F)(F)CN3[C@]3(C(=O)Nc4c(Cl)cc(Cl)cc43)[C@H]2C(=O)O)c1] | 636.1 | | ¹H NMR (400 MHz, DMSO-d₆): 12.40 (br s, 1H), 10.96 (s, 1H), 8.33 (d, J = 2.0 Hz, 1H), 7.47 (d, J = 2.0 Hz, 1H), 7.07-6.96 (m, 3H), 4.03 (d, J = 14.0 Hz, 1H), 3.87 (d, J = 7.6 Hz, 1H), 3.74-3.62 (m, 3H), 3.28-3.19 (m, 1H), 2.67-2.50 (m, 1H), 2.41-2.33 (m, 1H), 2.30 (br s, 6H), 2.24-2.07 (m, 1H), 0.88 (s, 9H), 0.82 (s, 3H), 0.70 (s, 3H). |
| 199 | 5-(tert-butyl)-N- methylthiazol-2-amine | 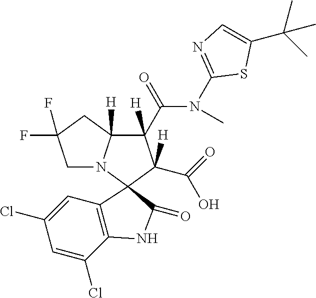 [SMILES: CN(C(=O)[C@H]1[C@H]2CC(F)(F)CN2[C@]2(C(=O)Nc3c(Cl)cc(Cl)cc32)[C@H]1C(=O)O)c1ncc(C(C)(C)C)s1] | 573.0 | | ¹H NMR (400 MHz, DMSO-d₆): 12.60 (br s, 1H), 11.10 (br s, 1H), 7.77 (br s, 1H), 7.53 (s, 1H), 7.28 (s, 1H), 4.18 (br s, 2H), 3.92-3.86 (m, 1H), 3.87 (s, 3H), 2.89-2.73 (m, 3H), 2.68-2.55 (m, 1H), 1.34 (s, 9H). |

Using either 110.4_1a or 110.4_1b and following the amide coupling procedures (as used to make 110.5a or 111.1b) and deprotection (110.6a or 111.2b), the following examples were made.

### Example 200

### Synthesis of (1′R,2′S,3R,7a′R)-5,7-dichloro-1-((3,5-dichlorophenyl)(methyl)carbamoyl)-6′,6′-difluoro-1-methyl-2-oxo-1′,2′,5′,6′,7′,7a′-hexahydrospiro[indoline-3,3′-pyrrolizine]-2′-carboxylic acid:

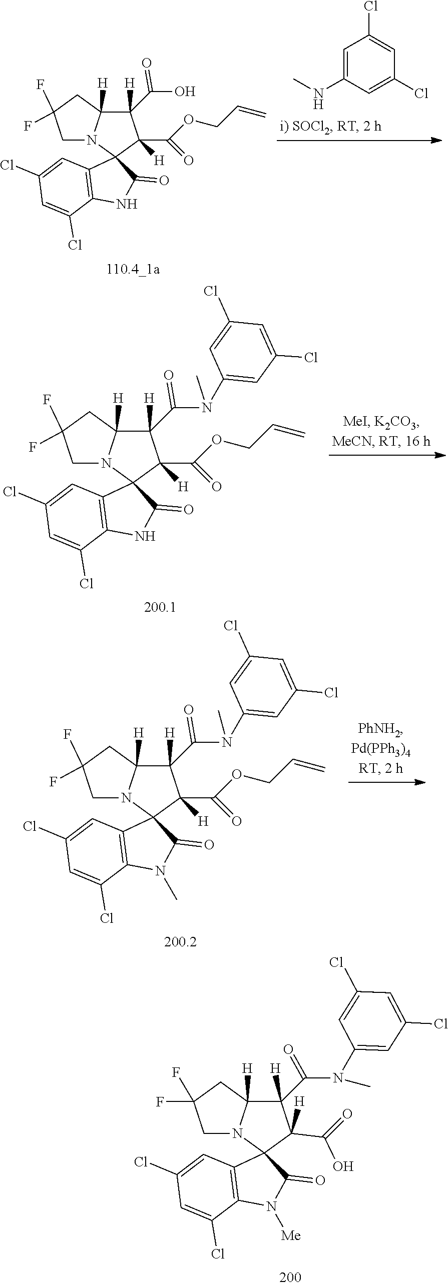

### Synthesis of 200.1

Thionyl chloride (6 mL) was added to 110.4_1a (300 mg, 0.65 mmol) at RT and stirred for 2 h. The excess thionyl chloride was concentrated under reduced pressure to afford the acid chloride derivative. To this acid chloride in CH₂Cl₂ (5 mL) was added a solution of 3,5-dichloro-N-methylaniline (228 mg, 1.3 mmol) in CH₂Cl₂ (5 mL). After stirring for 16 h at RT, the reaction mixture was quenched with water (10 mL) and the organic layer was separated. The aqueous layer was extracted with CH₂Cl₂ (2×10 mL). The combined organic layer was washed with brine, dried over anhydrous Na₂SO₄, filtered and concentrated under reduced pressure. The residue was purified by flash chromatography (40 g Silica gel cartridge, 20% EtOAc in pet ether) to afford 200.1 (280 mg, 69%) as a solid. LCMS: 62.1%, m/z [M+H]⁺=620.0.

### Synthesis of 200.2

To a stirred solution of 200.1 (280 mg, 0.45 mmol) in MeCN (10 mL) was added K₂CO₃ (62 mg, 0.45 mmol) followed by Mel (69 mg, 0.48 mmol) at RT. After stirring for 16 h at RT, the reaction mixture was quenched with water (10 mL) and extracted with EtOAc (15 mL). The organic layer was washed with water (15 mL), brine (15 mL), dried over anhydrous Na₂SO₄, filtered and concentrated under reduced pressure to afford 200.2 (270 mg) as a pale brown solid. The residue was used in the next step without purification. LCMS: 58.1%, m/z [M+H]⁺=634.2.

### Synthesis of 200

To a stirred solution of 200.2 (260 mg, 0.41 mmol) in THF (10 mL) were added aniline (38 mg, 0.41 mmol) and Pd(PPh₃)₄ (95 mg, 0.08 mmol) at RT. After stirring for 2 h at RT, the reaction mixture was concentrated under reduced pressure. The residue was triturated with diethyl ether: n-pentane. The resulting residue was purified by prep. HPLC [Column: X-BRIDGE C18 (150×30) mm, 5 μ; A: 0.1% Formic acid in H₂O, B: acetonitrile; Gradient: (Time/%B): 0/60, 8/85, 10/90, 10.1/98, 13/98, 13.1/60, 16/60 at 18 mL/min] to afford 200 (45 mg, 17%) as a solid.

¹H NMR (500 MHz, DMSO-d₆) (Exist in rotameric form): 12.53 (br s, 1H), 8.40/7.98 (d, J=2.0 Hz, 1H), 7.67-7.42 (m, 4H), 4.11-4.02 (m, 1H), 3.82-3.78 (m, 1H), 3.58-3.55 (m, 1H), 3.43 (s, 3H), 3.23 (s, 3H), 3.22-3.16 (m, 1H), 2.57-2.50 (m, 1H), 2.38-2.30 (m, 1H), 2.11-2.04 (m, 1H); LCMS: 95.0%, [M+H]⁺=592.0.

TABLE 6

| | | | M/Z | |
|---|---|---|---|---|
| Example | aniline | Compound | (M + H)⁺ | 1H NMR |
| 201 | 3,5-dichloro-N- methylaniline | 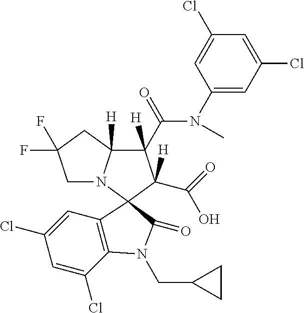 [SMILES: CN(C(=O)[C@H]1[C@H]2CC(F)(F)CN2[C@]2(C(=O)N(CC3CC3)c3c(Cl)cc(Cl)cc32)[C@H]1C(=O)O)c1cc(Cl)cc(Cl)c1] | 632.1 | (500 MHz, DMSO-d6): 12.79 (br s, 1H), 7.79-7.75 (m, 2H), 7.57-7.45 (m, 3H), 4.24- 4.22 (m, 1H), 3.91-3.88 (m, 1H), 3.83-3.73 (m, 3H), 3.45-3.42 (m, 1H), 3.25 (s, 3H), 2.71-2.66 (m, 1H), 2.50-2.37 (m, 1H), 2.28- 2.22 (m, 1H), 1.19-1.10 (m, 1H), 0.45-0.39 (m, 2H), 0.24-0.22 (m, 2H). |
| 202 | 3,5-dichloro-N- (cyclopropylmethyl) aniline | 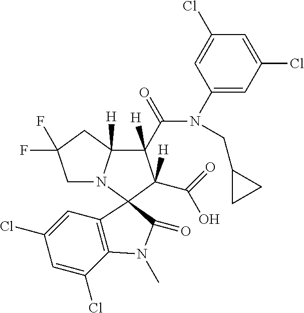 [SMILES: CN1C(=O)[C@]2(c3cc(Cl)cc(Cl)c31)[C@@H](C(=O)O)[C@@H](C(=O)N(CC1CC1)c1cc(Cl)cc(Cl)c1)[C@H]1CC(F)(F)CN12] | 632 | (500 MHz, DMSO-d₆) (Exist in rotameric form): 12.60 (s, 1H), 8.38/8.01 (d, J = 2.0 Hz, 1H), 7.69/7.60 (t, J = 1.5 Hz, 1H), 7.50 (d, J = 2.0 Hz, 1H), 7.46-7.39 (m, 2H), 4.03 (d, J = 7.5 Hz, 1H), 3.82-3.75 (m, 2H), 3.50- 3.42 (m, 5H), 3.22-3.19 (m, 1H), 2.53-2.50 (m, 1H), 2.39-2.36 (m, 1H), 2.07-2.20 (m, 1H), 0.89 (m, 1H), 0.42-0.40 (m, 2H), 0.17- 0.16 (m, 1H), 0.08-0.06 (m, 1H). |
| 203 | 3,5-dichloro-N- (cyclopropylmethyl) aniline | 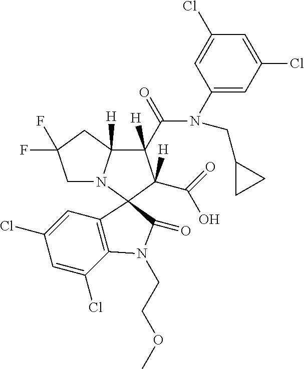 [SMILES: COCCN1C(=O)[C@]2(c3cc(Cl)cc(Cl)c31)[C@@H](C(=O)O)[C@@H](C(=O)N(CC1CC1)c1cc(Cl)cc(Cl)c1)[C@H]1CC(F)(F)CN12] | 675.9 | (500 MHz, DMSO-d₆) (Exist in rotameric form): 12.82 (br s, 1H), 7.79-7.78 (m, 2H), 7.56 (d, J = 2.0 Hz, 1H), 7.46/7.40 (br s, 2H), 4.23 (d, J = 9.5 Hz, 1H), 4.08-4.06 (m, 1H), 3.98-3.97 (m, 1H), 3.86-3.76 (m, 2H), 3.60 (d, J = 7.5 Hz, 2H), 3.44-3.39 (m, 3H), 3.21 (s, 3H), 2.69-2.60 (m, 1H), 2.60-2.52 (m, 1H), 2.38-2.18 (m, 1H), 0.90-0.88 (m, 1H), 0.44-0.41 (m, 2H), 0.10-0.09 (m, 2H). |
| 204 | 3,5-dichloro-N- (cyclopropylmethyl) aniline | 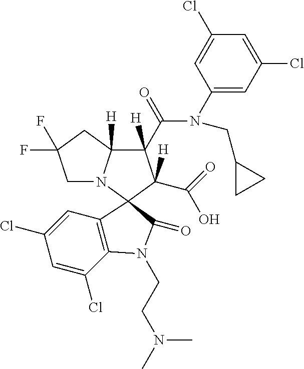 [SMILES: CN(C)CCN1C(=O)[C@]2(c3cc(Cl)cc(Cl)c31)[C@@H](C(=O)O)[C@@H](C(=O)N(CC1CC1)c1cc(Cl)cc(Cl)c1)[C@H]1CC(F)(F)CN12] | 689 | (500 MHz, DMSO-d₆): 7.74-7.73 (m, 1H), 7.64-7.57 (m, 2H), 7.53-7.50 (m, 2H), 4.28- 4.12 (m, 1H), 4.09-4.04 (m, 2H), 3.84-3.78 (m, 2H), 3.65-3.61 (m, 1H), 3.57-3.52 (m, 1H), 3.32-3.24 (m, 1H), 2.91-2.86 (m, 2H), 2.71-2.60 (m, 1H), 2.50 (s, 3H), 2.48 (s, 3H), 2.48-2.15 (m, 2H), 0.95-0.85 (m, 1H), 0.42- 0.39 (m, 2H), 0.10-0.06 (m, 2H). |
| 205 | 3,5-dichloro-N- (cyclopropylmethyl) aniline | 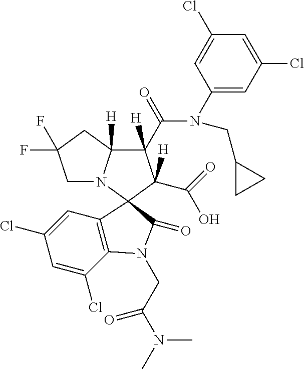 [SMILES: CN(C)C(=O)CN1C(=O)[C@]2(c3cc(Cl)cc(Cl)c31)[C@@H](C(=O)O)[C@@H](C(=O)N(CC1CC1)c1cc(Cl)cc(Cl)c1)[C@H]1CC(F)(F)CN12] | 702.9 | (500 MHz, DMSO-d₆) (Exist in rotameric form): 12.58 (br s, 1H), 8.38/8.03 (d, J = 2.0 Hz, 1H), 7.70/7.60 (t, J = 2.0 Hz, 1H), 7.44- 7.38 (m, 3H), 4.92 (d, J = 17.5 Hz, 1H), 4.78 (d, J = 17.5 Hz, 1H), 4.11-4.00 (m, 1H), 3.80-3.72 (m, 2H), 3.52 (t, J = 7.0 Hz, 1H), 3.45-3.41 (m, 1H), 3.06/3.05 (s, 3H), 3.10- 2.99 (m, 1H), 2.85/2.83 (s, 3H), 2.57-2.50 (m, 1H), 2.40-2.30 (m, 1H), 2.12-1.91 (m, 1H), 0.90-0.84 (m, 1H), 0.42-0.41 (m, 2H), 0.17-0.15 (m, 1H), 0.08-0.02 (m, 1H). |
| 206 | 3,5-dichloro-N- (cyclopentylmethyl) aniline | 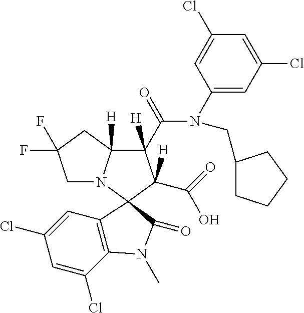 [SMILES: CN1C(=O)[C@]2(c3cc(Cl)cc(Cl)c31)[C@@H](C(=O)O)[C@@H](C(=O)N(CC1CCCC1)c1cc(Cl)cc(Cl)c1)[C@H]1CC(F)(F)CN12] | 660 | (500 MHz, DMSO-d₆) (Exist in rotameric form): 12.60 (br s, 1H), 8.38/7.98 (d, J = 2.5 Hz, 1H), 7.67 (s, 1H), 7.58-7.40 (m, 3H), 4.08-3.97 (m, 1H), 3.93-3.91 (m, 1H), 3.80- 3.78 (m, 1H), 3.53-3.49 (m, 1H), 3.45/3.43 (s, 3H), 3.22-3.18 (m, 1H), 2.60-2.50 (m, 1H), 2.42-2.31 (m, 1H), 2.12-2.00 (m, 1H), 1.92-1.89 (m, 1H), 1.65-1.57 (m, 5H), 1.48- 1.45 (m, 2H), 1.36-1.33 (m, 1H), 1.15-1.12 (m, 1H). |
| 207 | 3,5-dichloro-N- neopentylaniline | 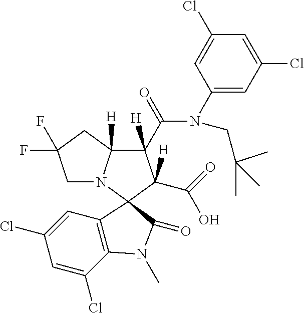 [SMILES: CN1C(=O)[C@]2(c3cc(Cl)cc(Cl)c31)[C@@H](C(=O)O)[C@@H](C(=O)N(CC(C)(C)C)c1cc(Cl)cc(Cl)c1)[C@H]1CC(F)(F)CN12] | 647.9 | (500 MHz, DMSO-d₆) (Exist in rotameric form): 12.63 (br s, 1H), 8.36/7.92 (d, J = 2.0 Hz, 1H), 7.61-7.46 (m, 4H), 4.07 (d, J = 7.5 Hz, 1H), 3.98 (d, J = 14 Hz, 1H), 3.82-3.78 (m, 1H), 3.64-3.61 (m, 1H), 3.48-3.45 (m, 1H), 3.43 (s, 3H), 3.28-3.19 (m, 1H), 2.56- 2.50 (m, 1H), 2.39-2.31 (m, 1H), 2.09-2.02 (m, 1H), 0.84/0.82 (s, 9H). |

Using 110.4_1a and the listed anilines, the following compounds were made as in Example 200.

### Example 208

### Synthesis of (1′R,2′S,3R,7a′R)-5,7-dichloro-1-cyclopropyl-1′-((cyclopropylmethyl)(3,5-dichlorophenyl)carbamoyl)-6′,6′-difluoro-2-oxo-1′,2′,5′,6′,7′,7a′-hexahydrospiro[indoline-3.3′-pyrrolizine]-2′-carboxylic acid:

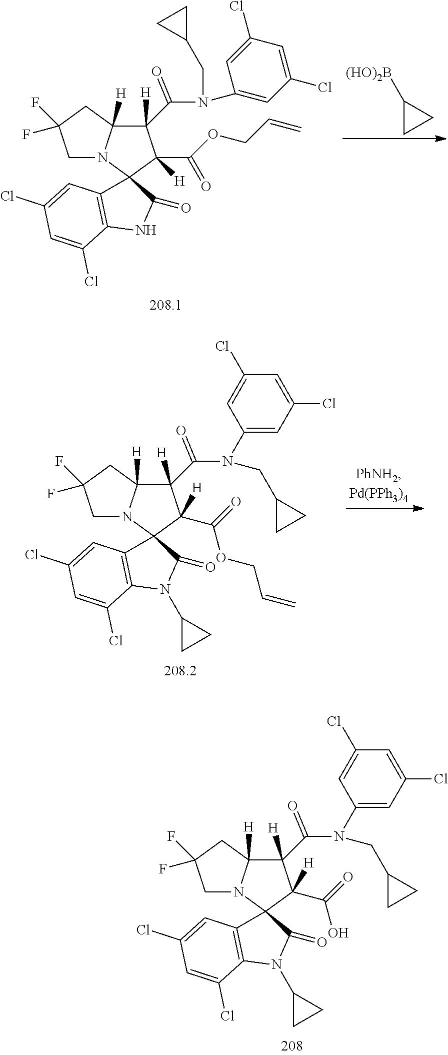 [SMILES: C=CCOC(=O)[C@H]1[C@@H](C(=O)N(CC2CC2)c2cc(Cl)cc(Cl)c2)[C@H]2CC(F)(F)CN2[C@]12C(=O)Nc1c(Cl)cc(Cl)cc12 ; OB(O)C1CC1 ; C=CCOC(=O)[C@H]1[C@@H](C(=O)N(CC2CC2)c2cc(Cl)cc(Cl)c2)[C@H]2CC(F)(F)CN2[C@]12C(=O)N(C1CC1)c1c(Cl)cc(Cl)cc12 ; O=C(O)[C@H]1[C@@H](C(=O)N(CC2CC2)c2cc(Cl)cc(Cl)c2)[C@H]2CC(F)(F)CN2[C@]12C(=O)N(C1CC1)c1c(Cl)cc(Cl)cc12]

### Synthesis of 208.1

208.1 was synthesized from 110.4_1a following procedure described for the synthesis of 200.1.

### Synthesis of 208.2

To a stirred solution of 208.1 (300 mg, 0.45 mmol) in DCM (10 mL) was added cyclopropylboronic acid (78 mg, 0.91 mmol) and TEA (0.12 mL, 0.91 mmol). After purging with oxygen for 10 minutes, Cu(OAc)₂ (82 mg, 0.45 mmol) was added and purged again with oxygen for 5 minutes. After stirring at RT for 16 h, the reaction mixture was diluted with DCM (20 mL), filtered through a small pad of Celite and the pad was washed with DCM (50 mL). The filtrate was washed with water (20 mL) and brine (20 mL). The combined organic layer was dried over anhydrous Na₂SO₄, filtered and concentrated under reduced pressure. The residue was purified by flash chromatography using (40 g Silica gel cartridge, 20% EtOAc in pet ether) to afford 208.2 (120 mg, 37%) as solid.

¹H NMR (400 MHz, CDCl₃): 8.32 (d, J=2.4 Hz, 1H), 7.41-7.40 (m, 1H), 7.30-7.26 (m, 1H), 7.24-7.17 (br s, 2H), 5.48-5.40 (m, 1H), 5.16-5.07 (m, 2H), 4.28-4.23 (m, 1H), 3.97-3.93 (m, 2H), 3.72-3.67 (m, 1H), 3.60-3.55 (m, 1H), 3.51-3.45 (m, 1H), 3.35-3.32 (m, 1H), 3.22-3.13 (m, 1H), 2.94-2.92 (m, 1H), 2.65-2.57 (m, 1H), 2.29-2.15 (m, 2H), 1.12-1.10 (m, 2H), 0.98-0.86 (m, 3H), 0.52-0.50 (m, 2H), 0.21-0.19 (m, 2H); LCMS: 98.6%, m/z [M+H]⁺=700.0.

### Synthesis of 208

To a stirred solution of 208.2 (100 mg, 0.14 mmol) in THF (10 mL) were added aniline (13 mg, 0.14 mmol) and Pd(PPh₃)₄ (33 mg, 0.02 mmol) at RT. After stirring for 2 h at RT, the reaction mixture was concentrated under reduced pressure. The residue was purified by prep. HPLC [Column: KROMOSIL-C18 (150×25 mm), 10 u; A: 0.1% Formic in H₂O, Acetonitrile; Gradient:(Time/%B): −0/60, 8/85, 12/95, 12.1/98, 14/98, 14.1/60, 16/60 at 22 mL/min] to afford 208 (80 mg, 85%) as a solid.

¹H NMR (500 MHz, DMSO-d₆) (Exist in rotameric form): 12.53 (br s, 1H), 8.36/7.98 (d, J=2.5 Hz, 1H), 7.68/7.60 (t, J=2.0 Hz, 1H), 7.51-7.38 (m, 3H), 4.12-3.98 (m, 1H), 3.79-3.75 (m, 2H), 3.44-3.40 (m, 2H), 3.18 (m, 1H), 2.96-2.94 (m, 1H), 2.51-2.50 (m, 1H), 2.41-2.32 (m, 1H), 2.15-1.93 (m, 1H), 1.13-0.96 (m, 2H), 0.88-0.85 (m, 2H), 0.80-0.65 (m, 1H), 0.42-0.40 (m, 2H), 0.17- 0.16 (m, 1H), 0.07-0.02 (m, 1H); LCMS: 98.2% , m/z [M+H]⁺=657.9.

### Example 216

### Synthesis of (1′S,2′R,3S,7a′S)-5-chloro-1′-((3,5-dichlorophenyl)(neopentyl)carbamoyl)-6′,6′,7-trifluoro-2-oxo-1′,2′,5′,6′,7′,7a′-hexahydrospiro[indoline-3,3′-pyrrolizine]-2′-carboxylic acid and (1′R,2′S,3R,7a′R)-5-chloro-1-((3,5-dichlorophenyl)(neopentyl)carbamoyl)-6′,6′,7-trifluoro-2-oxo-1′,2′,5′,6′,7′,7a′-hexahydrospiro[indoline-3,3′-pyrrolizine]-2′-carboxylic acid (216.8a and 216.8b):

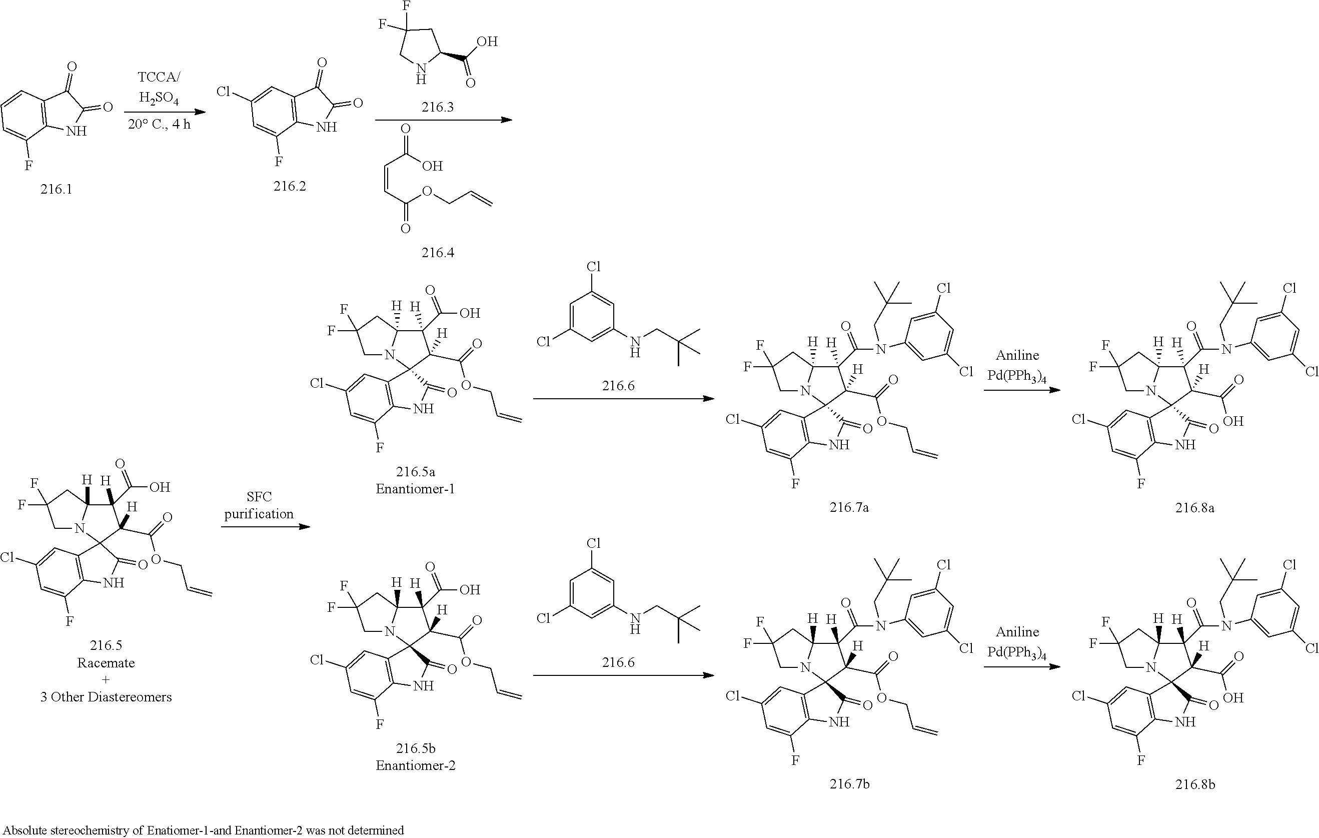

### Synthesis of 216.2

To a stirred solution of 216.1 (3.0 g, 18.2 mmol) in conc. H₂SO₄ (15 mL) and CH₃SO₃H (15 mL) was added trichloroisocyanuric acid (2.1 g, 9.08 mmol) at 0° C. After stirring for 4 h at RT, the reaction was cooled to 0° C. and quenched with ice cold H₂O (100 mL). The resulting precipitate was filtered, washed with H₂O (200 mL), collected and dried under vacuum to afford 216.2 (3.5 g, 97%) as an orange solid.

¹H NMR (400 MHz, DMSO-d₆): 11.66 (s, 1H), 7.77 (dd, J=2.0 Hz, 10.0 Hz, 1H), 7.47 (d, J=2.0 Hz, 1H); GCMS: 97.7%, m/z [M+H]⁺=200.8.

### Synthesis of 216.5a and 216.5b

To a stirred solution of 216.3 (3 g, 12.1 mmol) in MTBE (100 mL) were added 216.2 (2.4 g, 12.1 mmol) and 216.4 (1.88 g, 12.1 mmol) at RT. After stirring at 80° C. for 16 h, the reaction mixture was cooled to RT and then concentrated under reduced pressure. The diastereomeric mixture (dr 7%:26%:27%:5% by LCMS) was purified by column chromatography (Silica gel 100-200 mesh, 20-30% EtOAc/pet ether) to afford required diastereomer 216.5 (1.7 g, 32%) as an off white solid. Regio and relative stereochemistry were confirmed by 2D NMR studies.

¹H NMR (500 MHz, DMSO-d₆): 12.86 (br s, 1H), 11.23 (s, 1H), 7.58 (d, J=2.0 Hz, 1H), 7.42 (dd, J=1.5 Hz, 9.5 Hz, 1H), 5.50-5.45 (m, 1H), 5.11-5.06 (m, 2H), 4.28-4.27 (m, 2H), 4.09-4.05 (m, 1H), 4.01 (d, J=7.5 Hz, 1H), 3.65 (t, J=7.0 Hz, 1H), 3.21-3.18 (m, 1H), 2.69-2.68 (m, 1H), 2.50-2.46 (m, 1H), 2.19-2.09 (m, 1H); LCMS: 97.9%, m/z [M+H]⁺=445.0; Chiral purity: (50.5+49.4)%.

### Separation of 216.5a and 216.5b

216.5 (1.7 g) was separated by chiral SFC using Chiralcel OX-H (30×250) mm, 5 μ; A: 75% CO₂%, B: 25% (0.5% DEA in Methanol at RT (Isocratic 90 g/min, with detection at 214 nm). Pure fractions were concentrated under reduced pressure to afford 216.5a (Enantiomer-1, 760 mg, 89%) as an off-white solid and 216.5b (Enantiomer-2, 670 mg, 79%) as an off-white solid.

216.5a: ¹H NMR (500 MHz, DMSO-d₆): 12.88 (br s, 1H), 11.23 (s, 1H), 7.58 (d, J=1.5 Hz, 1H), 7.42 (dd, J=2.0 Hz, 10.0 Hz, 1H), 5.50-5.45 (m, 1H), 5.12-5.06 (m, 2H), 4.28-4.27 (m, 2H), 4.10-4.05 (m, 1H), 4.01 (d, J=7.5 Hz, 1H), 3.65 (t, J=7.0 Hz, 1H), 3.21-3.18 (m, 1H), 2.74-2.65 (m, 1H), 2.50-2.45 (m, 1H), 2.20-2.09 (m, 1H); LCMS: 97.5%, m/z [M+H]⁺=445.0; Chiral purity: 99.7%.

216.5b: ¹H NMR (500 MHz, DMSO-d₆): 12.86 (br s, 1H), 11.23 (s, 1H), 7.58 (d, J=2.0 Hz, 1H), 7.42 (dd, J=2.0 Hz, 10.0 Hz, 1H), 5.50-5.45 (m, 1H), 5.12-5.06 (m, 2H), 4.29-4.27 (m, 2H), 4.08-4.06 (m, 1H), 4.01 (d, J=7.5 Hz, 1H), 3.65 (t, J=7.0 Hz, 1H), 3.21-3.18 (m, 1H), 2.74-2.65 (m, 1H), 2.50-2.44 (m, 1H), 2.20-2.09 (m, 1H); LCMS: 98.7%, m/z [M+H]⁺=445.0; Chiral purity: 99.8%.

### Synthesis of 216.7a

To 216.5a (200 mg, 0.44 mmol) was added SOCl₂ (3 mL). After stirring for 2 h at RT, SOCl₂ was evaporated under reduced pressure to afford intermediate acid chloride. To above prepared intermediate acid chloride at RT was added solution of 216.6 (150 mg, 0.64 mmol) in CH₂Cl₂. After stirring at 50° C. for 16 h, the reaction mixture was evaporated and resulting residue was purified by flash column chromatography (24 g Silica gel cartridge, 15% EtOAc in pet ether) to afford 216.7a (150 mg, 53%) as an off-white solid.

¹H NMR (400 MHz, CDCl₃): 8.18 (d, J=2.0 Hz, 1H), 7.60 (br s, 1H), 7.34 (t, J=1.6 Hz, 1H), 7.26 (s, 1H), 7.08 (dd, J=2.0 Hz, 9.6 Hz, 1H), 5.50-5.42 (m, 1H), 5.15-5.06 (m, 2H), 4.37-4.32 (m, 1H), 4.25-4.20 (m, 1H), 4.05-3.98 (m, 2H), 3.81-3.68 (m, 2H), 3.54-3.49 (m, 1H), 3.34-3.29 (m, 1H), 2.75-2.70 (m, 1H), 2.31-2.10 (m, 2H), 0.92 (s, 9H); LCMS: 96.1%, m/z [M+H]⁺=660.0.

### Synthesis of 216.8a

To a stirred solution of compound 216.7a (130 mg, 0.19 mmol) in THF (3 mL) were added aniline (18 mg, 0.19 mmol) and Pd(PPh₃)₄ (46 mg, 0.04 mmol) at RT. After stirring for 1 h, the reaction mixture was diluted with EtOAc (15 mL). The organic solution was collected, washed with brine (10 mL), dried over anhydrous Na₂SO₄, filtered and concentrated under reduced pressure. The residue was purified by prep. HPLC [Column: X-BRIDGE-C18 (150×30) mm, 5 μ, A: 0.1% Formic Acid in H₂O, B: Acetonitrile; Gradient: (Time/%B): 0/70, 8/90, 9/90, 9.1/98, 11/98, 11.1/70, 14/70 at 20 mL/min] to afford 216.8a (47 mg, 38%) as solid.

¹H NMR (500 MHz, DMSO-d₆) (exist in rotameric form): 12.59/12.45 (s, 1H), 11.10/11.06 (s, 1H), 8.16/7.71 (d, J=1.5 Hz, 1H), 7.61-7.34 (m, 4H), 4.06-4.00 (m, 2H), 3.84-3.80 (m, 1H), 3.61-3.58 (m, 1H), 3.44 (d, J=14 Hz, 1H), 3.36-3.29 (m, 1H), 2.59-2.50 (m, 1H), 2.38-2.34 (m, 1H), 2.13-2.06 (m, 1H), 0.85/0.82 (s, 9H); LCMS: 98.1%, m/z [M+H]⁺=618.0; Chiral Purity: 99.9%.

### Synthesis of 216.7b

To 216.5b (200 mg, 0.44 mmol) was added SOCl₂ (3 mL). After stirring for 2 h at RT, SOCl₂ was evaporated under reduced pressure to afford intermediate acid chloride. To above prepared intermediate acid chloride at RT was added a solution of compound 216.6 (150 mg, 0.64 mmol) in CH₂Cl₂. After stirring at 50° C. for 16 h, the reaction mixture was evaporated and the residue was purified by flash column chromatography (24 g Silica gel cartridge, 15% EtOAc in pet ether) to afford 216.7b (180 mg, 63%) as an off white solid.

¹H NMR (400 MHz, CDCl₃): 8.18 (d, J=2.0 Hz, 1H), 7.65 (br s, 1H), 7.34 (t, J=1.6 Hz, 1H), 7.26 (s, 1H), 7.08 (dd, J=1.6 Hz, 9.2 Hz, 1H), 5.50-5.42 (m, 1H), 5.14-5.06 (m, 2H), 4.37-4.32 (m, 1H), 4.25-4.20 (m, 1H), 4.05-3.98 (m, 2H), 3.81-3.68 (m, 2H), 3.54-3.50 (m, 1H), 3.36-3.27 (m, 1H), 2.76-2.68 (m, 1H), 2.31-2.05 (m, 2H), 0.92 (s, 9H); LCMS: 90.2%, m/z [M+H]⁺=660.0.

### Synthesis of 216.8b:

To a stirred solution of compound 216.7b (160 mg, 0.24 mmol) in THF (3 mL) were added aniline (23 mg, 0.24 mmol) and Pd(PPh₃)₄ (56 mg, 0.05 mmol) at RT. After stirring for 1 h, the reaction mixture was diluted with EtOAc (15 mL). The organic solution was collected, washed with brine (10 mL), dried over anhydrous Na₂SO₄, filtered and concentrated under reduced pressure. The residue was purified by prep. HPLC [Column: X-BRIDGE-C18 (150×30) mm, 5 μ, A: 0.1% Formic Acid in H₂O, B: Acetonitrile; Gradient: (Time/%B): 0/60, 8/90, 10/90, 10.1/98, 11/98, 11.1/60, 14/60 at 20 mL/min] to afford 216.8b (93 mg, 62%) as solid.

¹H NMR (500 MHz, DMSO-d₆) (exist in rotameric form): 12.59/12.45 (s, 1H), 11.10/11.06 (s, 1H), 8.16/7.71 (d, J=1.5 Hz, 1H), 7.61-7.34 (m, 4H), 4.06-4.00 (m, 2H), 3.85-3.80 (m, 1H), 3.60 (t, J=7.0 Hz, 1H), 3.44 (d, J=14 Hz, 1H), 3.36-3.28 (m, 1H), 2.59-2.50 (m, 1H), 2.38-2.34 (m, 1H), 2.11-2.06 (m, 1H), 0.85/0.82 (s, 9H); LCMS: 97.4%, m/z [M+H]⁺=618.0; Chiral Purity: 99.6%.

TABLE 7

| | | | M/Z | |
|---|---|---|---|---|
| Example | Isatin | Compound | (M + H)⁺ | 1H NMR |
| 217a | 7-chloro-5- fluoroindoline- 2,3-dione | 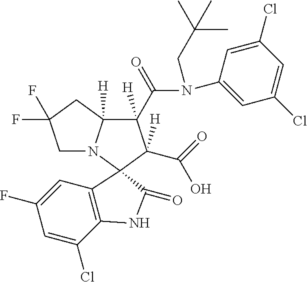 [SMILES: CC(C)(C)CN(C(=O)[C@H]1[C@@H](C(=O)O)[C@]2(C(=O)Nc3c(Cl)cc(F)cc32)N2CC(F)(F)C[C@@H]12)c1cc(Cl)cc(Cl)c1] | 618.0 | ¹H NMR (500 MHz, DMSO-d₆) (Exist in rotamcric form): 12.56/12.50 (br s, 1H), 10.90/10.86 (s, 1H), 8.10 (d, J = 7.0 Hz, 1H), 7.66-7.45 (m, 3H), 7.33/7.28 (d, J = 8.0 Hz, 1H), 4.05 (d, J = 7.5 Hz, 1H), 3.97 (d, J = 14.0 Hz, 1H), 3.84-3.80 (m, 1H), 3.62-3.61 (m, 1H), 3.49 (d, J = 14.0 Hz, 1H), 3.32-3.21 (m, 1H), 2.60-2.50 (m, 1H), 2.41-2.33 (m, 1H), 2.15-2.03 (m, 1H), 0.84/0.82 (s, 9H). |
| 217b | | 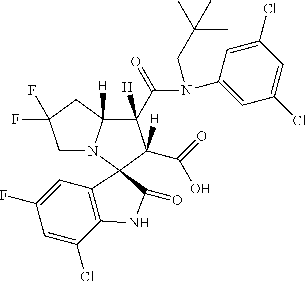 [SMILES: CC(C)(C)CN(C(=O)[C@H]1[C@H]2CC(F)(F)CN2[C@]2(C(=O)Nc3c(Cl)cc(F)cc32)[C@H]1C(=O)O)c1cc(Cl)cc(Cl)c1] | 618.1 | ¹H NMR (500 MHz, DMSO-d₆) (Exist in rotameric form): 12.56/12.50 (br s, 1H), 10.90/10.86 (s, 1H), 8.10 (dd, J = 2.0 Hz, 9.0 Hz, 1H), 7.66-7.44 (m, 3H), 7.33/7.28 (d, J = 8.0 Hz, 1H), 4.05 (d, J = 7.5 Hz, 1H), 3.97 (d, J = 14.0 Hz, 1H), 3.84-3.80 (m, 1H), 3.63-3.60 (m, 1H), 3.49 (d, J = 14.0 Hz, 1H), 3.32-3.21 (m, 1H), 2.60-2.50 (m, 1H), 2.41-2.33 (m, 1H), 2.15-2.03 (m, 1H), 0.84/0.82 (s, 9H). |
| 218a | 5,7- difluoroindoline- 2,3-dione | 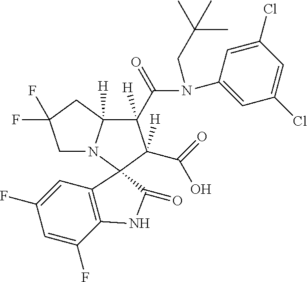 [SMILES: CC(C)(C)CN(C(=O)[C@H]1[C@@H](C(=O)O)[C@]2(C(=O)Nc3c(F)cc(F)cc32)N2CC(F)(F)C[C@@H]12)c1cc(Cl)cc(Cl)c1] | 602.1 | ¹H NMR (400 MHz. DMSO-d₆) (Exist in rotameric form): 12.55/12.45 (br s, 1H), 10.98/10.93 (s, 1H), 7.99 (dd, J = 2.0 Hz, 8.8 Hz, 1H), 7.61-7.44 (m, 3H), 7.26- 7.21 (m, 1H), 4.04 (d, J = 7.6 Hz, 1H), 3.97 (d, J = 14.0 Hz, 1H), 3.85-3.80 (m, 1H), 3.63-3.60 (m, 1H), 3.49 (d, J = 13.6 Hz, 1H), 3.32-3.26 (m, 1H), 2.60-2.50 (m, 1H), 2.42-2.32 (m, 1H), 2.14-2.05 (m, 1H), 0.84/0.83 (s, 9H). |
| 218b | | 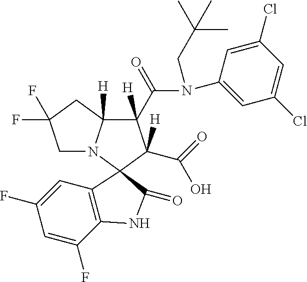 [SMILES: CC(C)(C)CN(C(=O)[C@H]1[C@H]2CC(F)(F)CN2[C@]2(C(=O)Nc3c(F)cc(F)cc32)[C@H]1C(=O)O)c1cc(Cl)cc(Cl)c1] | 602.1 | ¹H NMR (400 MHz, DMSO-d₆) (Exist in rotameric form): 12.54 (br s, 1H), 10.98/10.93 (s, 1H), 7.99 (dd, J = 2.0 Hz, 8.8 Hz, 1H), 7.61-7.44 (m, 3H), 7.26- 7.21 (m, 1H), 4.04 (d, J = 7.6 Hz, 1H), 3.97 (d, J = 14.0 Hz, 1H), 3.85-3.80 (m, 1H), 3.61-3.60 (m, 1H), 3.49 (d, J = 13.6 Hz, 1H), 3.32-3.26 (m, 1H), 2.60-2.50 (m, 1H), 2.42-2.32 (m, 1H), 2.15-2.04 (m, 1H), 0.84/0.83 (s, 9H). |

The following examples was made as in Example 216 with the listed isatins in place of 5-chloro-7-fluoroindoline-2,3-dione, 216.2. Regiochemistry and relative stereochemistry was assigned by 2D NMR studies. Absolute stereochemistry unknown for enantiomeric pairs (a and b).

### Example 219

### Synthesis of rac-(1′R,2′S,3R,8a′R)-5,7-dichloro-1′-((3,5-dichlorophenyl)(methyl)carbamoyl)-2-oxo-1′,5′,6′,7′,8′,8a′-hexahydro-2′H-spiro[indoline-3,3′-indolizine]-2′-carboxylic acid:

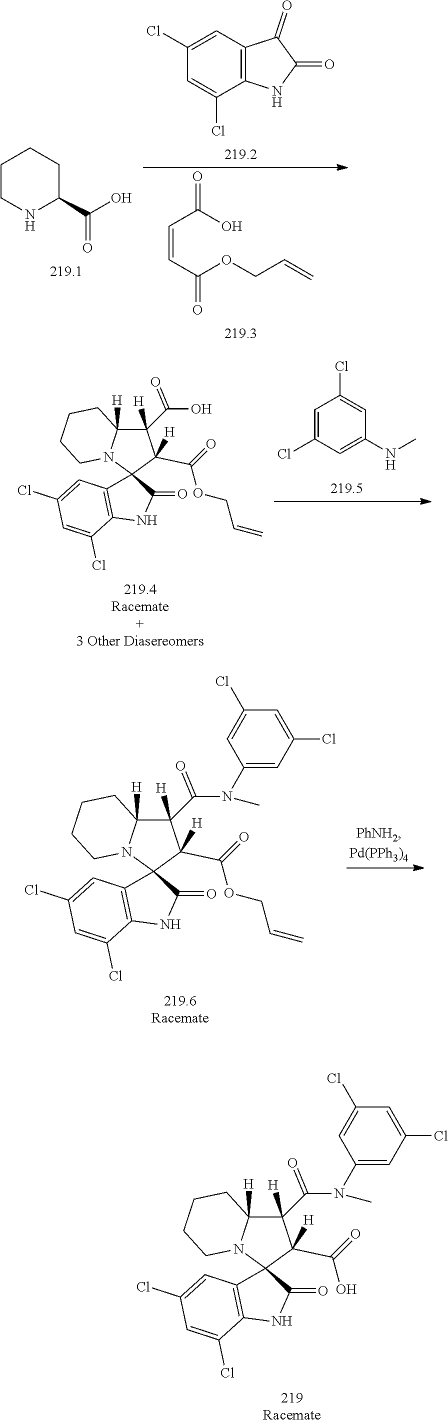

### Synthesis of 219.4

To a solution of 219.1 (3.0 g, 23.2 mmol) in acetontrile (50 mL) were added 219.2 (5.0 g, 23.2 mmol) and 219.3 (3.62 g, 23.2 mmol) at RT. After stirring at 90° C. for 16 h, the reaction mixture was concentrated. The residue was purified by column chromatography (Silica gel 100-200 mesh, 30% EtOAc/pet ether) to afford a mixture of diastereomers (dr=7:10:10:2). The mixture of diasteromers was purified by prep. HPLC [Column: X-SELECT-C18 (150×30) mm, 5 μ, A: 0.1% Formic Acid in H₂O, B: Acetonitrile; Gradient: (Time/%B): 0/30, 8/70, 11/70, 12/98, 12.1/98, 15/9 at 22 mL/min] to afford 219.4 (200 mg, 2%) as an off-white solid. The regio chemistry and relative stereochemistry was confirmed by 2D NMR analysis.

¹H NMR (500 MHz, DMSO-d₆, at 100° C.): 11.93 (br s, 1H), 10.62 (br s, 1H), 7.85 (d, J=2.0 Hz, 1H), 7.28 (d, J=2.0 Hz, 1H), 5.51-5.45 (m, 1H), 5.08-5.03 (m, 2H), 4.23-4.21 (m, 2H), 3.72 (d, J=7.5 Hz, 1H), 3.36-3.33 (m, 1H), 3.27-3.24 (m, 1H), 2.29-2.20 (m, 2H), 1.83-1.72 (m, 2H), 1.48-1.46 (m, 1H), 1.22-1.17 (m, 3H); LCMS: 95.5%, m/z [M+H]⁺=439.0; Chiral purity: (50.8+49.2)%.

### Synthesis of 219.6

Thionyl chloride (2 mL) was added to 219.4 (110 mg, 0.25 mmol) at RT. After stirring for 2 h, the thionyl chloride was evaporated under reduced pressure to afford acid chloride. To the above intermediate acid chloride at RT was added a solution of 219.5 (66 mg, 0.38 mmol) in CH₂Cl₂ (3 mL). After stirring at 50° C. for 16 h, the reaction mixture was concentrated under reduced pressure. The residue was purified by column chromatography (Silica gel 100-200 mesh, 20% EtOAc/pet ether) to afford 219.6 (30 mg, 20%) as a light brown solid. LCMS: 69.9%, m/z [M+H]⁺=598.0.

### Synthesis of 219

To a stirred solution of 219.6 (30 mg, 0.05 mmol) in THF (2 mL) were added aniline (5 mg, 0.05 mmol) and Pd(PPh₃)₄ (11 mg, 0.01 mmol) at RT. After stirring at RT for 2 h, the reaction mixture was concentrated under reduced pressure. The residue was purified by prep. HPLC [Column: KROMOSIL-C18 (150×25) mm, 10 μ; A: 0.1% Formic Acid in H₂O, B: Acetonitrile; Gradient: (Time/%B): 0/60, 8/80, 9/80, 9.1/98, 12/98, 12.1/60, 14/60 at 22 mL/min] to afford 219 (3 mg, 10%) as an off-white solid.

¹H NMR (400 MHz, DMSO-d₆) (exist in rotameric form): 12.40/12.30 (s, 1H), 10.94/10.90 (s, 1H), 8.32/8.01 (d, J=2.0 Hz, 1H), 7.63/7.53 (t, J=1.6 Hz, 1H), 7.46-7.35 (m, 3H), 3.64 (d, J=7.6 Hz, 1H), 3.53 (t, J=6.0 Hz, 1H), 3.43/3.22 (s, 3H), 3.01-2.99 (m, 1H), 2.20-2.13 (m, 2H), 1.68-1.66 (m, 1H), 1.54-1.51 (m, 1H), 1.43-1.40 (m, 1H), 1.19-1.05 (m, 3H); LCMS: 90.1%, m/z [M+H]⁺=556.1; Chiral purity: (46.7+45.2)%.

### Example 220

### Synthesis of 5,7-dichloro-8′-((3,5-dichlorophenyl)(neopentyl)carbamoyl)-2-oxo-3′,4′,7′,8′,8a′-hexahydrospiro[indoline-3,6′-pyrrolo[2,1-c][1,4]oxazine]-7′-carboxylic acid 220.7a and 220.7c:

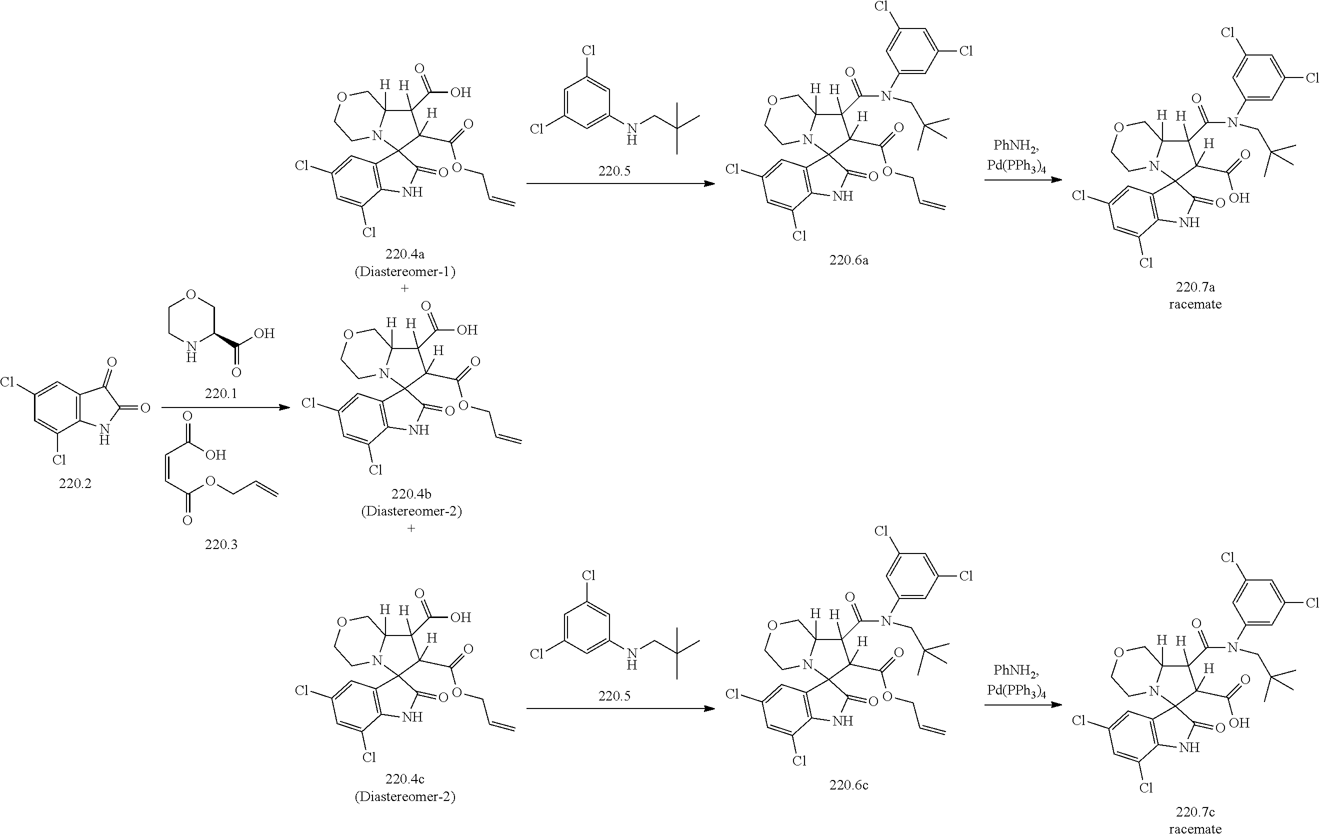

### Synthesis of 220.4a, 220.4b & 220.4c

To a stirred solution of 220.1 (3.0 g, 22.9 mmol) in THF (100 mL) were added 220.2 (4.94 g, 22.9 mmol), 220.3 (3.57 g, 22.9 mmol) and DIPEA (2.95 g, 22.9 mmol) at RT. After stirring at 80° C. for 16 h, the reaction mixture was diluted with water (30 mL) and extracted with EtOAc (2×50 mL). The combined organic layer was washed with brine (25 mL), dried over anhydrous Na₂SO₄, filtered and concentrate under reduced pressure to afford crude mixture of diastreomers (dr=5+3+6%). The crude mixture of diastereomers was purified by column chromatography (Silica gel 100-200 mesh, 30% EtOAc in pet ether) to afford 220.4a (250 mg), 220.4b (60 mg), 220.4c (450 mg) as solids.

220.4a: ¹H NMR (500 MHz, Acetone-d6): 11.11 (s, 1H), 9.90 (s, 1H), 7.98 (d, J=2.0 Hz, 1H), 7.33 (d, J=2.0 Hz, 1H), 5.61-5.52 (m, 1H), 5.14-5.05 (m, 2H), 4.31-4.28 (m, 2H), 4.09-4.06 (m, 1H), 3.85 (d, J=7.0 Hz, 1H), 3.71-3.68 (m, 1H), 3.62-3.55 (m, 2H), 3.29-3.24 (m, 2H), 2.60-2.59 (m, 1H), 2.28-2.26 (m, 1H); LCMS: 93.0%, m/z [M+H]⁺441.0.

220.4b: LCMS: 80%, m/z [M+H]⁺=441.1

220.4c: LCMS: 68%, m/z [M+H]⁺=441.1

### Synthesis of 220.6a

SOCl₂(5 mL) was added to 220.4a (250 mg, 0.59 mmol) at RT. After stirring for 2 h, the reaction mixture was concentrated under reduced pressure to afford intermediate acid chloride. To above acid chloride was added a solution of 220.5 (158 mg, 0.68 mmol) in DCM (10 mL). After stirring at 55° C. for 16 h, the reaction mixture was concentrated under reduced pressure. The residue was purified by column chromatography (Silica gel 100-200 mesh, 30% EtOAc in pet ether) to afford 220.6_1 (40 mg, 11%) as solid.

¹H NMR (500 MHz, Acetone-d6): 9.82 (s, 1H), 8.43 (d, J=1.5 Hz, 1H), 7.58-7.54 (m, 2H), 7.48 (d, J=2 Hz, 1H), 7.33 (d, J=2.0 Hz, 1H), 5.63-5.50 (m, 1H), 5.15-5.11 (m, 2H), 4.42-4.40 (m, 1H) 4.29-4.21 (m, 1H), 3.89-3.83 (m, 4H), 3.62-3.60 (m, 2H), 3.31-3.24 (m, 3H), 2.52-2.49 (m, 1H), 2.25-2.23 (m, 1H), 0.91 (s, 9H); LCMS: 93.5%, m/z [M+H]⁺=656.

### Synthesis of 220.7a

To a stirred solution of 220.6a (40 mg, 0.06 mmol) in THF (2 mL) were added aniline (6 mg, 0.06 mmol) and Pd(PPh₃)₄ (14 mg, 0.01 mmol) at RT. After stirring for 2 h at RT, the reaction mixture was concentrated under reduced pressure. The resulting residue was purified by prep. HPLC [Column: KROMOSIL-C18 (150×25) mm, 10 μ; A: 0.1% Formic Acid in H₂O, B: Acetonitrile; Gradient: (Time/%B): 0/60, 8/70, 8.1/98, 9/98, 9.1/60, 12/60 at 25 mL/min] to afford 220.7_1 (3.0 mg, 8%) as solid.

LCMS: 95.1%, m/z [M+H]⁺=614.1

### Synthesis of 220.6c

SOCl₂ (10 mL) was added to 220.4c (450 mg, 1.02 mmol) at RT. After stirring under nitrogen atmosphere for 2 h, the reaction mixture was concentrated under reduced pressure to afford intermediate acid chloride. To the above prepared acid chloride was added 220.5 (273 mg, 1.18 mmol) in DCM (15 mL). After stirring at 55° C. for 16 h, the reaction mixture was concentrated under reduced pressure. The residue was purified by silica gel column chromatography (100-200 Silica gel, 30% EtOAc/pet ether) to afford 220.6c (250 mg, 37%) as an orange solid.

¹H NMR (400 MHz, DMSO-d₆): 11.24 (s, 1H), 7.81-7.68 (m, 3H), 7.49 (d, J=2.0 Hz, 1H), 6.65 (s, 1H), 5.34-5.26 (m, 1H), 5.06-5.00 (m, 2H), 4.26-4.21 (m, 1H), 4.15-3.97 (m, 3H), 3.87 (d, J=10.0 Hz, 1H), 3.57 (d, J=10.0 Hz, 1H), 3.27-3.03 (m, 4H), 2.50-2.45 (m, 2H), 2.15-2.11 (m, 1H), 0.80 (s, 9H). LCMS: 85.3%, m/z [M+H]⁺=656.

### Synthesis of 220.7c

To a stirred solution of 220.6a (250 mg, 0.38 mmol) in THF (10 mL) were added aniline (35 mg, 0.38 mmol) and Pd(PPh₃)₄ (88 mg, 0.08 mmol) at RT. After stirring for 2 h at RT, the reaction mixture was concentrated under reduced pressure. The residue was purified by prep. HPLC [Column: X-SELECT-C₁₈ (150×19) mm, 5 μ; A: 0.1% Formic acid in H₂O, B: Acetonitrile; Gradient: (Time/%B): 0/10, 8/80, 11/89, 11.1/98, 13.98, 15/98 at 18 mL/minute] to afford 220.7c (35 mg, 15%) as solid.

¹H NMR (500 MHz, DMSO-d₆): 12.80 (s, 1H), 11.18 (s, 1H), 7.79 (s, 2H), 7.74 (s, 1H), 7.48 (s, 1H), 6.68 (s, 1H), 4.14 (d, J=14.0 Hz, 1H), 3.94 (m, J=10.0 Hz, 1H), 3.79 (d, J=10.5 Hz, 1H), 3.56 (d, J=9.5 Hz, 1H), 3.30-3.20 (m, 2H), 3.09-3.01 (m, 2H), 2.50-2.46 (m, 1H), 2.42-2.36 (m, 1H), 2.10-2.07 (m, 1H), 0.77 (s, 9H). LCMS: 97.7%, m/z [M+H]⁺=614.0; Chiral purity: (51.5+48.4)%.

### Example 225

### Synthesis of 5,7-dichloro-N1′-(3,5-dichlorophenyl)-6′,6′-difluoro-N2′-hydroxy-N2′-methyl-2-oxo-1′,2′,5′,6′,7′,7a′-hexahydrospiro[indoline-3,3′-pyrrolizine]-1′,2′-dicarboxamide (225a and 225b)

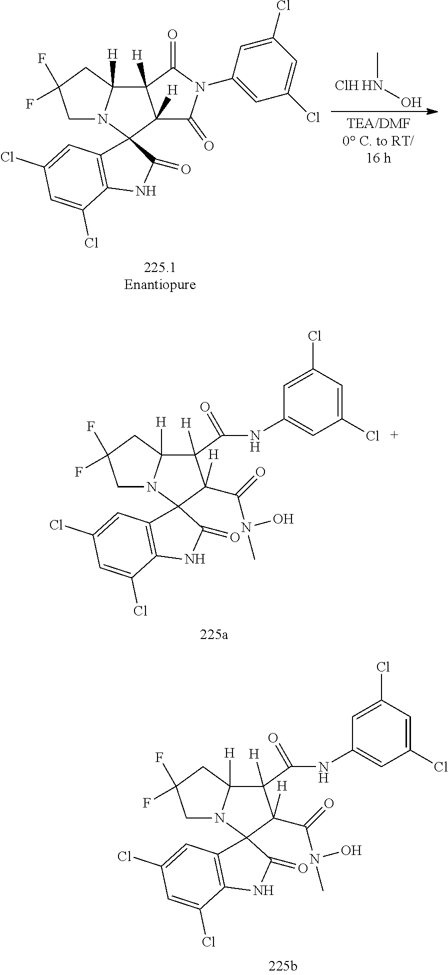

To a stirred solution of 225.1 (350 mg, 0.64 mmol) in DMF (15 mL) were added TEA (1.33 mL, 9.58 mmol) and N-methylhydroxylamine hydrochloride (534 mg, 6.39 mmol) at 0° C. in sealed tube. After warming slowly allowed to RT and stirring for 16 h, the reaction mixture was diluted with EtOAc (20 mL) and washed with H₂O (2×20 mL). The organic layer was dried over anhydrous Na₂SO₄, filtered and concentrated under reduced pressure. The residue was purified by column chromatography followed by SFC purification [Column: Chiralpak IC (4.6×250) mm, 5 μ; 75% CO₂: 25% Methanol at RT (Isocratic 20 mL/min, with detection at 214 nm)] to afford 225a (20 mg, 5%) as an off-white solid and 225b (45 mg, 12%) as an off-white solid. Absolute stereochemistry was not established for 225a and 225b.

225a: ¹H NMR (500 MHz, DMSO-d₆): 11.20 (br s, 1H), 10.06 (br s, 1H), 10.03 (br s, 1H), 7.55 (d, J=2.0 Hz, 1H), 7.41 (d, J=1.5 Hz, 2H), 7.29 (t, J=2.0 Hz, 1H), 7.10 (br s, 1H), 4.58-4.52 (m, 1H), 4.47-4.42 (m, 1H), 3.83-3.74 (m, 1H), 3.34-3.32 (m, 1H), 3.08 (s, 3H), 3.01-2.90 (m, 1H), 2.83-2.78 (m, 1H), 2.37-2.31 (m, 1H); LCMS: 94.8%, m/z [M+H]⁺=593.1.

225b: ¹H NMR (500 MHz, DMSO-d₆): 10.85 (br s, 1H), 10.32 (s, 1H), 9.86 (br s, 1H), 7.68 (d, J=2.0 Hz, 3H), 7.49 (d, J=2.0 Hz, 1H), 7.30 (t, J=2.0 Hz, 1H), 4.25 (d, J=7.5 Hz, 1H), 4.13-4.09 (m, 1H), 3.49-3.38 (m, 2H), 2.69 (s, 3H), 2.60-2.50 (m, 1H), 2.47-2.39 (m, 1H), 2.26-2.13 (m, 1H); LCMS: 95.8%, m/z [M+H]⁺=593.1.

### Example 226

### Synthesis of 5,7-dichloro-N1′-(3,5-dichlorophenyl)-6′,6′-difluoro-N2′-hydroxy-2-oxo-1′,2′,5′,6′,7′,7a′-hexahydrospiro[indoline-3,3′-pyrrolizine]-1′,2′-dicarboxamide (226a and 226b)

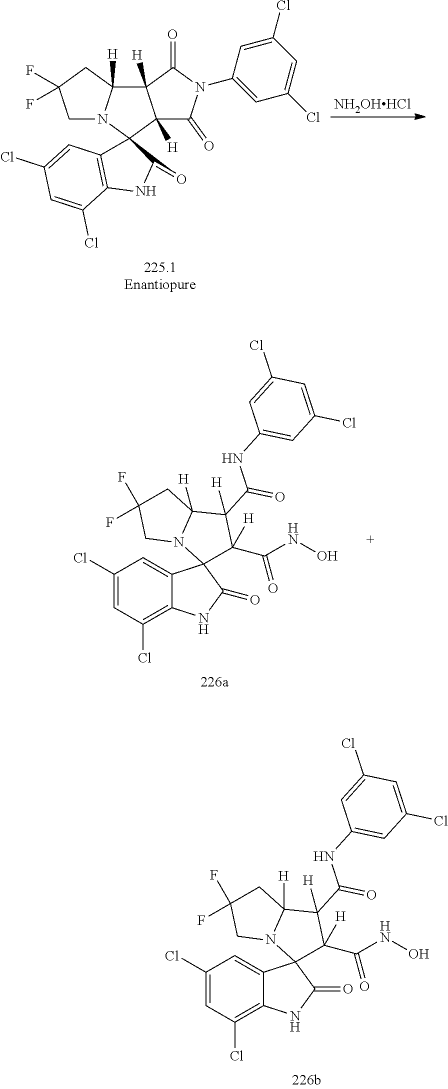

To a stirred solution of 225.1 (200 mg, 0.37 mmol) in DMF (3 mL) were added NH₂OH.HCl (127 mg, 1.83 mmol) and TEA (0.25 mL, 1.83 mmol) at RT. After stirring for 4 h at RT, the reaction mixture was poured into ice cold water (15 mL) and stirred well for 10 minutes. The resulting precipitate was filtered, washed with cold water (10 mL) and dried under high vacuum. The residue was purified by prep. HPLC [Column: KROMASIL-C18 (150×25) mm, 10 μ; A: 0.1% Formic acid in H₂O, B: Acetonitrile; Gradient: (Time/%B): 0/45, 8/70, 9.4/70, 9.5/98, 11/98, 11.1/45, 13/45 at 25 mL/min] to afford 226a (30 mg, 14%) as a white solid and 226b (17 mg, 8%) as a white solid. Absolute stereochemistry was not established for 226a and 226b.

226a: ¹H NMR (500 MHz, DMSO-d₆): 11.14 (br s, 1H), 10.53 (br s, 1H), 10.06 (s, 1H), 8.88 (s, 1H), 7.53 (d, J=2.0 Hz, 1H), 7.44-7.35 (m, 2H), 7.30-7.29 (m, 2H), 4.29-4.24 (m, 1H), 4.00-3.97 (m, 1H), 3.65-3.60 (m, 1H), 3.50 (d, J=7.5 Hz, 1H), 3.00-2.81 (m, 1H), 2.79-2.76 (m, 1H), 2.34-2.27 (m, 1H); LCMS: 97.5%, m/z [M−H]⁻=576.9.

226b: ¹H NMR (500 MHz, DMSO-d₆): 11.08 (br s, 1H), 10.40 (br s, 1H), 10.17 (br s, 1H), 8.92 (s, 1H), 7.70-7.65 (m, 2H), 7.57 (d, J=2.0 Hz, 1H), 7.42 (d, J=1.5 Hz, 1H), 7.29 (t, J=2.0 Hz, 1H), 4.40-4.35 (m, 1H), 4.24-4.21 (m, 1H), 3.79-3.64 (m, 1H), 3.26 (d, J=8.0 Hz, 1H), 3.10-2.95 (m, 1H), 2.75-2.70 (m, 1H), 2.37-2.28 (m, 1H); LCMS: 95.6%, m/z [M−H]⁻=576.9.

### Example 227

### Synthesis of (1′R,2′S,3R,7a′R)-5,7-dichloro-N1′-(3,5-dichlorophenyl)-6′,6′-difluoro-N2′-methoxy-N2′-methyl-2-oxo-1′,2′,5′,6′,7′,7a′-hexahydrospiro [indoline-3,3′-pyrrolizine]-1′,2′-dicarboxamide

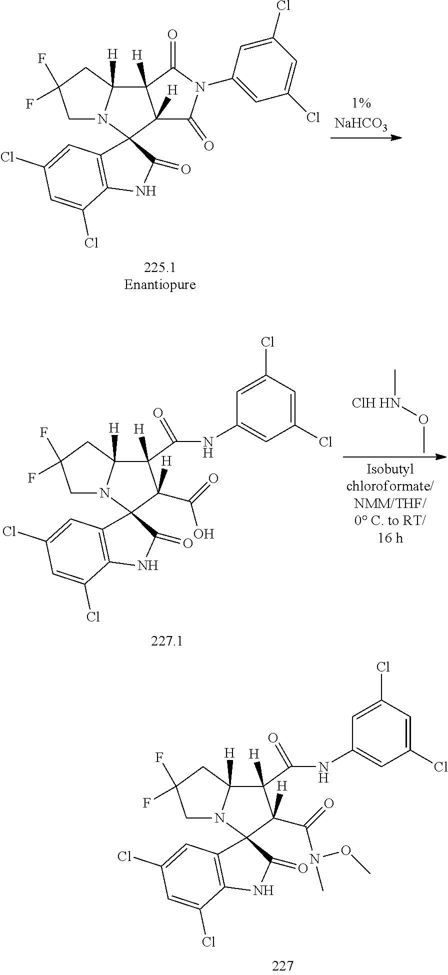

To a stirred solution of 227.1 (300 mg, 0.53 mmol) in THF (10 mL) were added N-methylmorpholine (87 μL, 0.79 mmol) and isobutyl chloroformate (62 μL, 0.64 mmol) at 0° C. After stirring for 5 minutes, N,O-dimethylhydroxylamine hydrochloride (103 mg, 1.06 mmol) was added at 0° C. After stirring for 16 h at RT, the reaction mixture was concentrated under reduced pressure. The residue was purified by prep. HPLC [Column: SYMMETRY-C₈ (300×19) mm, 7 u; A: 0.1% Formic acid in H₂O, B: Acetonitrile; Gradient: (T%B): −0/50, 8/80, 8.1/98, 10/98, 10.1/50, 13/50 at 20 mL/min] followed by normal phase prep. HPLC [Column: Chiracel OX—H (250×30) mm, 5 u, Mobile Phase: Acetonitrile at RT (Isocratic 42.0 mL /min, with detection at 215 nm)] to afford 227 (59 mg, 18%) as a white solid.

¹H NMR (400 MHz, DMSO-d₆): 10.89 (s, 1H), 10.38 (s, 1H), 7.70-7.68 (m, 3H), 7.51 (d, J=2.0 Hz, 1H), 7.31 (t, J=2.0 Hz, 1H), 4.26 (d, J=7.2 Hz, 1H), 4.13 (dd, J=7.2 Hz, J=6.8 Hz, 1H), 3.54-3.40 (m, 2H), 3.44 (s, 3H), 2.67 (s, 3H), 2.61-2.54 (m, 1H), 2.49-2.46 (m, 1H), 2.25-2.16 (m, 1H); LCMS: 98.3%, m/z [M+H]⁺=606.9.

### Example 228

### Synthesis of (1′R,2′S,3R,7a′R)-5,7-dichloro-N1′-(3,5-dichlorophenyl)-6′,6′-difluoro-2-oxo-1′,2′,5′,6′,7′,7a′-hexahydrospiro[indoline-3,3′-pyrrolizine]-1′,2′-dicarboxamide

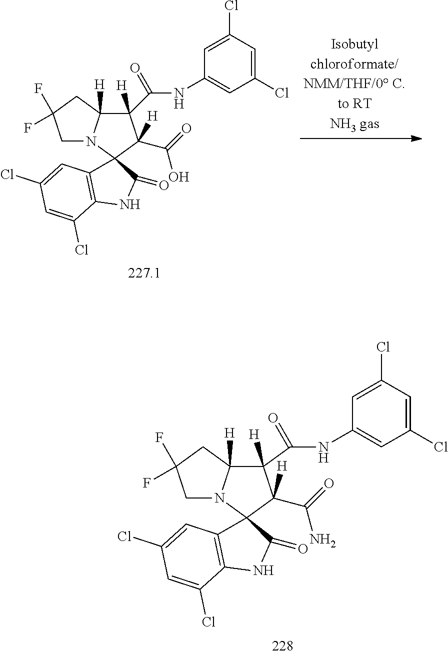 [SMILES: O=C(Nc1cc(Cl)cc(Cl)c1)[C@H]1[C@H]2CC(F)(F)CN2[C@]2(C(=O)Nc3c(Cl)cc(Cl)cc32)[C@H]1C(=O)O ; NC(=O)[C@H]1[C@@H](C(=O)Nc2cc(Cl)cc(Cl)c2)[C@H]2CC(F)(F)CN2[C@]12C(=O)Nc1c(Cl)cc(Cl)cc12]

To a stirred solution of 227.1 (200 mg, 0.35 mmol) in THF (20 mL) were added N-methylmorpholine (58 μL, 0.53 mmol) and isobutyl chloroformate (41 μL, 0.42 mmol) at 0° C. After stirring for 5 minutes, NH₃ gas was purged into the reaction mixture for 10 minutes at 0° C. After stirring for 16 h at RT, the reaction mixture was concentrated under reduced pressure. The residue was purified by prep. HPLC [Column: X-BRIDGE C18 (250×19) mm, 5; A: 0.1% Formic acid in H₂O, B: Acetonitrile; Gradient: (T%B): −0/50, 8/80, 8.1/98, 10/98, 10.1/50, 13/50 at 20 mL/min] to afford 228 (96 mg, 47%) as a white solid.

¹H NMR (500 MHz, DMSO-d₆): 10.89 (s, 1H), 10.74 (s, 1H), 7.71-7.70 (m, 3H), 7.53 (d, J=2.0 Hz, 1H), 7.33 (t, J=2.0 Hz, 1H), 7.00 (br s, 1H), 6.66 (br s, 1H), 4.55-4.50 (m, 1H), 4.25 (t, J=10.5 Hz, 1H), 3.99 (d, J=11.0 Hz, 1H), 3.49-3.41 (m, 1H), 2.84 (t, J=11.5 Hz, 1H), 2.41-2.35 (m, 1H), 2.16-2.06 (m, 1H); LCMS: 96.9%, m/z [M+H]⁺=562.9.

### Example 229

### Synthesis of rel-(1′R,2′S,3R,7a′R)-5,7-dichloro-N1′-(3,5-dichlorophenyl)-6′,6′-difluoro-N2′-hydroxy-N1′,N2′-dimethyl-2-oxo-1′,2′,5′,6′,7′,7a′-hexahydrospiro[indoline-3,3′-pyrrolizine]-1′,2′-dicarboxamide and 5,7-dichloro-N1′-(3,5-dichlorophenyl)-6′,6′-difluoro-N2′-hydroxy-N1′,N2′-dimethyl-2-oxo-1′,2′,5′,6′,7′,7a′-hexahydrospiro[indoline-3,3′-pyrrolizine]-1′,2′-dicarboxamide (229a and 229b):

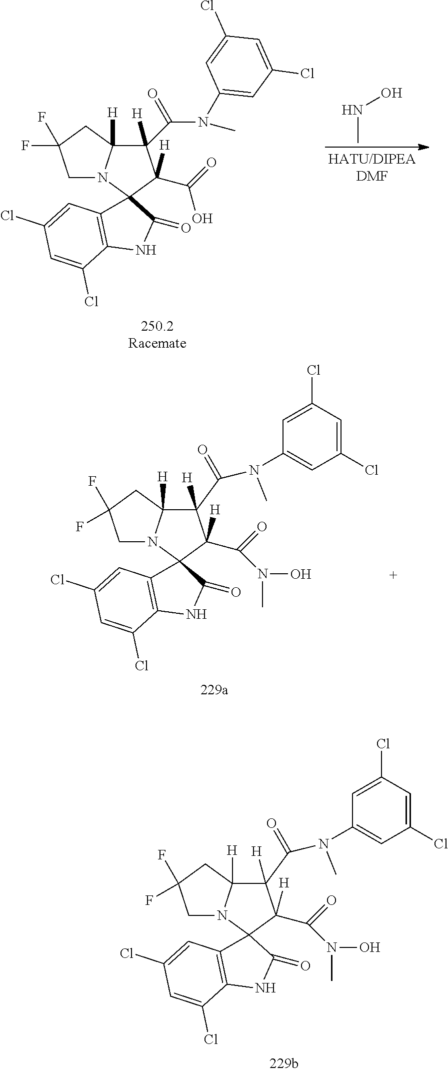

229a and 229b were synthesized from 250.2 following the procedure described for the synthesis of 260a and 260b. Absolute stereochemistry was not established for 229a and 229b.

229a: ¹H NMR (500 MHz, DMSO-d₆) (Exist in rotameric form): 10.88/10.79 (s, 1H), 10.07/9.95 (s, 1H), 7.72/7.54 (s, 1H), 7.51-7.30 (m, 4H), 4.55/4.42 (d, J=8.0 Hz, 1H), 4.17-4.05 (m, 1H), 3.51-3.36 (m, 2H), 3.36/3.22 (s, 3H), 2.99/2.97 (s, 3H), 2.74-2.50 (m, 2H), 2.23-2.10 (m, 1H); LCMS: 96.0%, m/z [M+H]⁺=607.0.

229b: ¹H NMR (500 MHz, DMSO-d₆) (Exist in rotameric form): 10.75/10.69 (s, 1H), 9.90/9.82 (s, 1H), 7.95/7.82 (s, 1H), 7.77/7.63 (s, 1H), 7.60-7.45 (m, 3H), 4.22/4.07 (d, J=7.5 Hz, 1H), 3.85-3.77 (m, 1H), 3.56-3.35 (m, 2H), 3.24 (s, 3H), 2.72 (s, 3H), 2.50-2.32 (m, 2H), 2.22-2.10 (m, 1H); LCMS: 95.1%, m/z [M+H]⁺=607.0.

### Example 230

### Synthesis of rel-(1′R,2′S,3R,7a′R)-5,7-dichloro-N1′-(3,5-dichlorophenyl)-6′,6′-difluoro-N1′-methyl-2-oxo-N2′-(phenylsulfonyl)-1′,2′,5′,6′,7′,7a′-hexahydrospiro[indoline-3,3′-pyrrolizine]-1′,2′-dicarboxamide

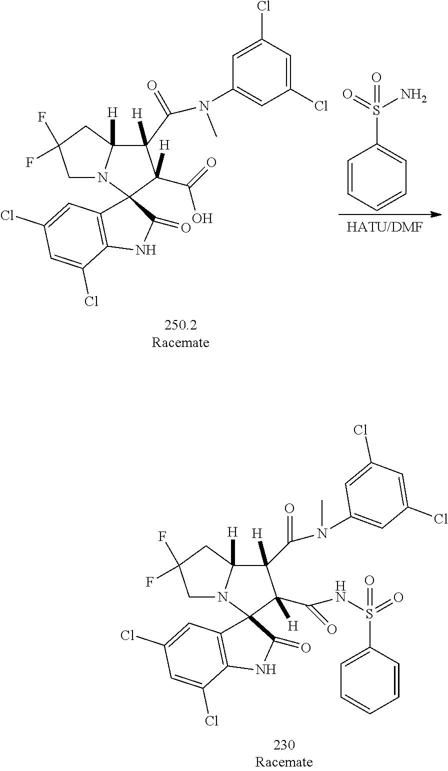

To a stirred solution of 250.2 (200 mg, 0.34 mmol) in DMF (5 mL) were added DIPEA (0.12 mL, 0.69 mmol) and HATU (196 mg, 0.51 mmol) at RT. After stirring 15 minutes, benzenesulfonamide (81 mg, 0.51 mmol) was added. After stirring for 12 h at RT, the reaction mixture was quenched with ice cold water (5 mL) and extracted with EtOAc (2×10 mL). The combined organic layer was washed with brine (10 mL), dried over anhydrous Na₂SO₄, filtered and concentrated. The resulted residue was purified by prep. HPLC [Column: X-BRIDGE-C18 (150×30) mm, 5 μ; A: 0.1% Formic acid in H₂O, B: Acetonitrile; Gradient: (Time/%B): 0/40, 8/80, 11/90, 11.1/98, 12/98, 12.1/40, 15/40 at 23 mL/min] to afford 230 (24 mg, 10%) as an off-white solid.

¹H NMR (500 MHz, DMSO-d₆) (exist in rotameric form): 12.00 (br s, 1H), 10.86/10.66 (s, 1H), 7.74-7.39 (m, 10H), 4.60/4.21 (m, 1H), 4.00-3.92 (m, 1H), 3.67-3.63 (m, 1H), 3.27-3.23 (m, 1H), 3.20 (s, 3H), 2.96-2.87 (m, 1H), 2.64-2.50 (m, 1H), 2.14-2.02 (m, 1H); LCMS: 93.0%, m/z [M-H]⁻=715.0.

### Example 231

### Synthesis of rel-(1′R,2′S,3R,7a′R)-5,7-dichloro-N1′-(3,5-dichlorophenyl)-6′,6′-difluoro-N1′-methyl-N2′-(methylsulfonyl)-2-oxo-1′,2′,5′,6′,7′,7a′-hexahydrospiro[indoline-3,3′-pyrrolizine]-1′,2′-dicarboxamide.

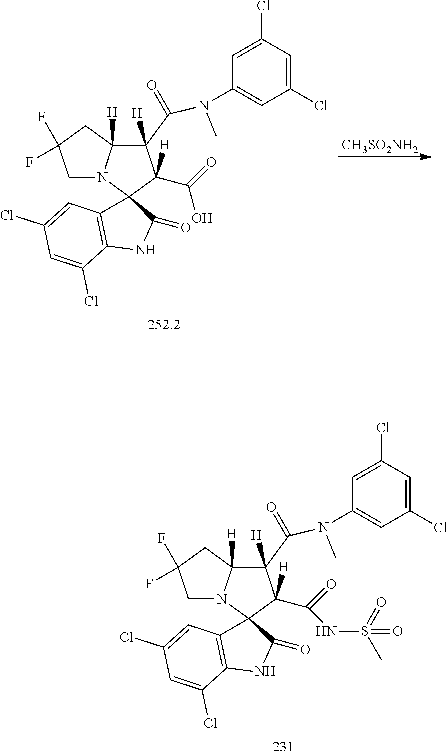

To a stirred solution of 252.2 (1.0 g, 1.72 mmol) in DMF (15 mL) were added DIPEA (0.47 mL, 2.59 mmol) and HATU (0.98 g, 2.59 mmol) at RT. After stirring for 30 minutes, methansulphonamide (0.27 g, 2.59 mmol) was added. After stirring for 16 h at RT, the reaction mixture was quenched with water (20 mL) and extracted with EtOAc (2×20 mL). The combined organic layer was washed with brine (20 mL), dried over anhydrous Na₂SO₄, filtered and concentrated under reduced pressure. The residue was purified by flash column chromatography (40 g Silica gel cartridge, 30% EtOAc in pet ether) followed by prep. HPLC [Column: X-BRIDGE-C18 (150×30) mm, 5 μ; A: 0.1% Formic acid in H₂O, B: Acetonitrile; Gradient: (Time/%B): 0/50, 8/80, 9/80, 9.1/98, 10/98, 10.1/50, 12/50 at 23 mL/min] to afford 231 (71 mg, 6%) as an off-white solid.

¹H NMR (500 MHz, DMSO-d₆) (exist in rotameric form): 11.32 (br s, 1H), 11.10/11.93 (s, 1H), 7.75-7.48 (m, 5H), 4.58/4.25 (m, 1H), 4.00 (d, J=10.5 Hz, 1H), 3.75-3.71 (m, 1H), 3.39-3.33 (m, 1H), 3.22 (s, 3H), 3.01/2.98 (s, 3H), 2.92-2.86 (m, 1H), 2.50-2.43 (m, 1H), 2.15-2.07 (m, 1H); LCMS: 99.0, m/z [M+H]⁺=655.0; Chiral purity: 96.4%.

### Example 232

### Synthesis of (1′S,2′R,3S,7a′S)-5,7-dichloro-N1′-(3,5-dichlorophenyl)-6′,6′-difluoro-N1′-methyl-2-oxo-N2′-(2,2,2-trifluoroethyl)-1′,2′,5′,6′,7′,7a′-hexahydrospiro[indoline-3,3′-pyrrolizine]-1′,2′-dicarboxamide (232.1a) and (1′R,2′S,3R,7a′R)-5,7-dichloro-N1′-(3,5-dichlorophenyl)-6′,6′-difluoro-Nr-methyl-2-oxo-N2′-(2,2,2-trifluoroethyl)-1′,2′,5′,6′,7′,7a′-hexahydrospiro[indoline-3,3′-pyrrolizine]-1,2′-dicarboxamide (232.1b)

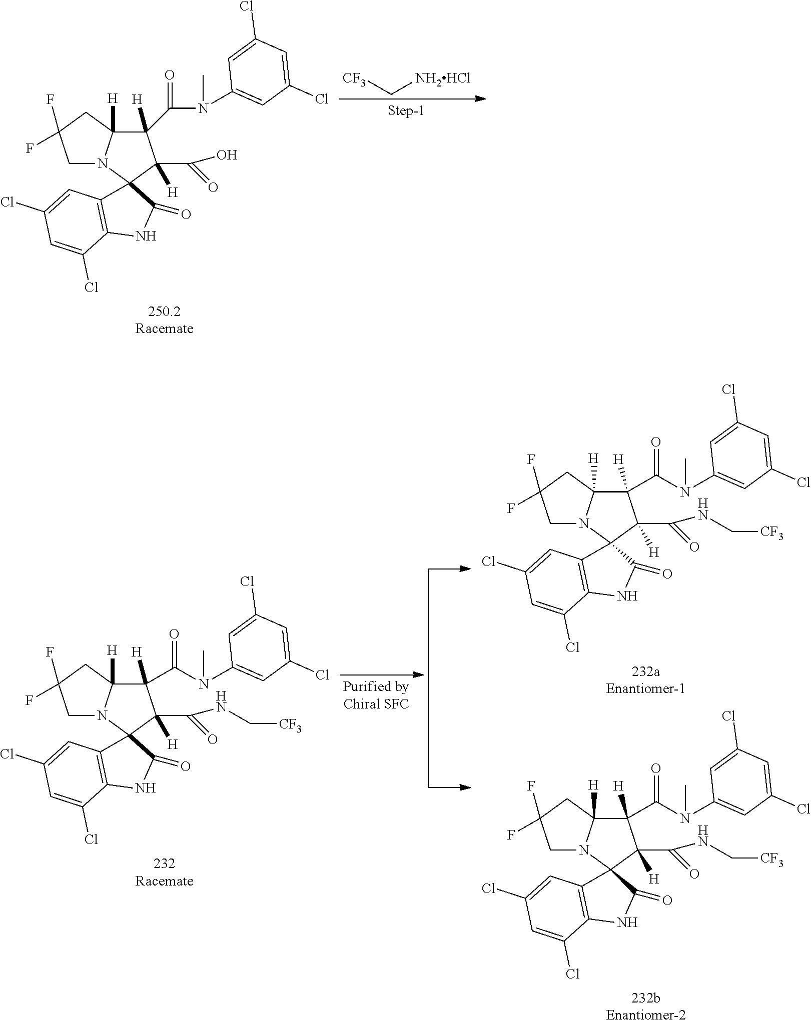

To a stirred solution of 250.2 (200 mg, 0.34 mmol) in DMF (5 mL) were added HATU (196 mg, 0.52 mmol) and Et₃N (0.14 mL, 1.04 mmol) at RT. After stirred for 15 minutes, 2,2,2-trifluoroethanamine hydrochloride (92 mg, 0.69 mmol) was added. After stirring for 3 h at RT, the reaction mixture was diluted with cold water (50 mL) and extracted with EtOAc (2×60 mL). The combined organic layer was washed with brine (50 mL), dried over anhydrous Na₂SO₄, filtered and concentrated under reduced pressure. The residue was purified by prep. HPLC [KROMOSIL-C18 (150×25) mm, 10 μ; A: 0.1% Formic Acid in H₂O, B: Acetonitrile; Gradient:(T%B): −0/30, 8/80, 11/85, 11.1/98, 13/98, 13.1/30, 15/30 at 22 mL/min] to afford 232 (110 mg, 50%) as an off-white solid.

¹H NMR (500 MHz, DMSO-d₆) (Exist in rotameric form): 10.95/10.74 (s, 1H), 7.82-7.45 (m, 6H), 4.26 (t, J=10.5 Hz, 1H), 4.00-3.97 (m, 1H), 3.88-3.84 (m, 1H), 3.77-3.72 (m, 1H), 3.65-3.60 (m, 1H), 3.46-3.39 (m, 1H), 3.39/3.21 (s, 3H), 2.89-2.85 (m, 1H), 2.60-2.50 (m, 1H), 2.25-2.10 (m, 1H); LCMS: 96.4%, m/z [M−H]⁻=657.0; Chiral purity: (49.9%+50.0%).

### Separation of 232a and 232b:

232 (100 mg) was separated by chiral SFC [Column: (R,R) Whelk-01 (30×250 mm), 5 μ; 90% CO₂: 10% Acetonitrile at RT (Isocratic 70 g/min, with detection at 214 nm)] to afford 232a (Enantiomer-1, 17 mg, 34%) as an off-white solid and 232b (Enantiomer-2, 20 mg, 40%) as an off white solid. Absolute stereochemistry was not determined.

232a: ¹H NMR (500 MHz, DMSO-d₆) (Exist in rotameric form): 10.95/10.74 (s, 1H), 7.82-7.44 (m, 6H), 4.26 (t, J=10 Hz, 1H), 3.99-3.97 (m, 1H), 3.90-3.84 (m, 1H), 3.77-3.72 (m, 1H), 3.67-3.62 (m, 1H), 3.46-3.39 (m, 1H), 3.39/3.21 (s, 3H), 2.89-2.85 (m, 1H), 2.60-2.50 (m, 1H), 2.25-2.10 (m, 1H); LCMS: 99.1%, m/z [M−H]⁻=657.0; Chiral purity: 98.65%.

232b: ¹H NMR (500 MHz, DMSO-d₆) (Exist in rotameric form): 10.95/10.74 (s, 1H), 7.76-7.44 (m, 6H), 4.26 (t, J=10 Hz, 1H), 3.99-3.97 (m, 1H), 3.90-3.84 (m, 1H), 3.77-3.72 (m, 1H), 3.65-3.62 (m, 1H), 3.48-3.37 (m, 1H), 3.41/3.21 (s, 3H), 2.89-2.85 (m, 1H), 2.60-2.50 (m, 1H), 2.25-2.10 (m, 1H); LCMS: 99.1%, m/z [M−H]⁻=657.0; Chiral purity: 99.7%.

### Example 233

### Synthesis of (1′R,2′S,7a′R)-5,7-dichloro-N1′-(3,5-dichlorophenyl)-N2′-methoxy-2-oxo-1′,2′,5′,6′,7′,7a′-hexahydrospiro[indoline-3,3′-pyrrolizine]-1,2′-dicarboxamide

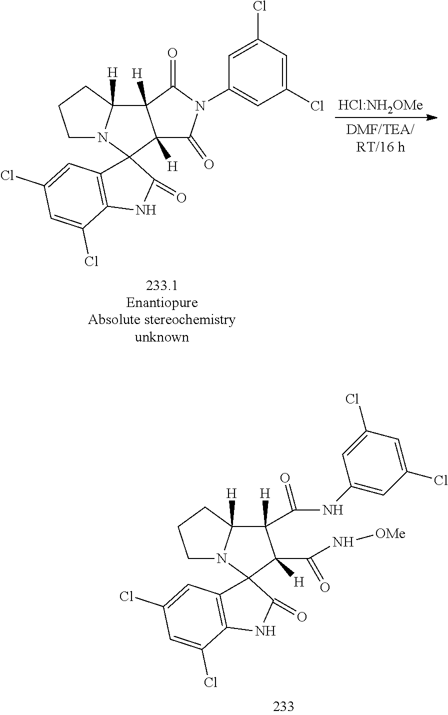

To a stirred solution of 233.1 (WO2017117239) (200 mg, 0.39 mmol) in DMF (15 mL) were added methoxyamine hydrochloride (326 mg, 3.9 mmol) and TEA (1.6 mL, 11.7 mmol) at RT. After stirring for 16 h at RT, the reaction mixture was quenched with ice cold water and extracted with EtOAc (2×30 mL). The combined organic layer was dried over Na₂SO₄, filtered and concentrated under reduced pressure. The residue was purified by prep. HPLC [Column: X-BRIDGE-C8 (150×19) mm, 5 μ; A: 0.1% Formic acid in H₂O, B: Acetonitrile; Gradient: (T%B): −0/40, 8/80, 9/80, 9.1/98, 11/98, 11.1/40, 14/40 at 25 mL/min] to obtain 233 (7 mg, 3%) as an off-white solid.

¹H NMR (400 MHz, DMSO-d₆): 10.96 (br s, 1H), 10.79 (br s, 1H), 10.12 (br s, 1H), 7.67 (s, 2H), 7.57-7.49 (m, 2H), 7.26 (t, J=2.0 Hz, 1H), 4.22-4.10 (m, 1H), 4.10-4.01 (m, 1H) 3.40 (s, 3H), 3.25-3.12 (m, 2H), 2.07-1.98 (m, 1H), 1.97-1.65 (m, 4H); LCMS: 94.6%, m/z [M+H]⁺=557.0. Regiochemistry is unknown.

### Example 234

### Synthesis of 5,7-dichloro-N1′-(3,5-dichlorophenyl)-N2′-hydroxy-2-oxo-1′,2′,5′,6′,7′,7a′-hexahydrospiro[indoline-3,3′-pyrrolizine]-1,2′-dicarboxamide (234a and 234b)

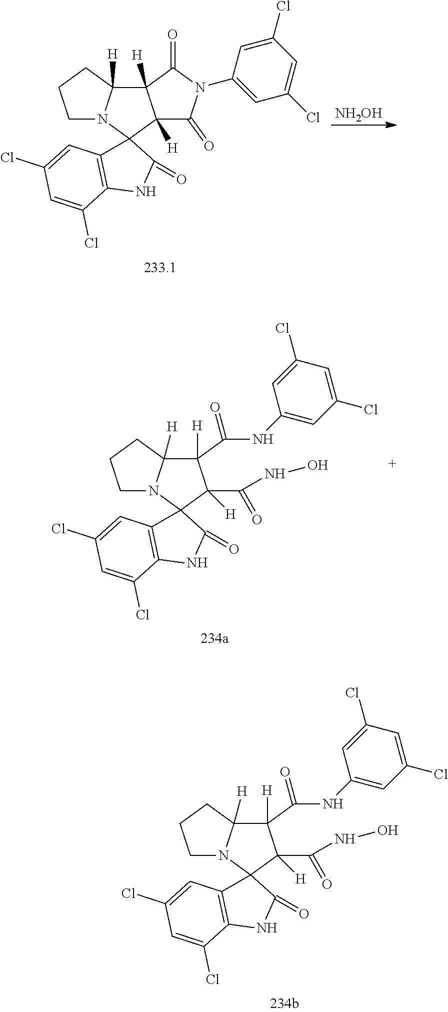

To a stirred solution of 233.1 (200 mg, 0.39 mmol) in DMF (5 mL) were added NH₂OH (137 mg, 1.96 mmol) and NEt₃ (0.27 mL, 1.96 mmol) at RT. After stirring for 16 h at RT, the reaction mixture was poured into ice cold water (15 mL) and stirred well. The resulting precipitate was filtered, washed with water (10 mL) and dried under high vacuum. The residue was purified by prep. HPLC [Column: KROMASIL-C18 (150×25) mm, 10 μ; A: 0.1% Formic acid in H₂O, B: Acetonitrile; Gradient: (T%B): −0/10, 9/80, 9.1/98, 11/98, 11.1/10, 13/10 at 25 mL/min] to obtain 234a (40 mg, 19%) as an off-white solid and 234b (150 mg, 71%) as an off white solid.

234a: ¹H NMR (500 MHz, DMSO-d₆): 10.99 (br s, 1H), 10.44 (br s, 1H), 9.88 (s, 1H), 8.81 (s, 1H), 7.47 (d, J=2.0 Hz, 2H), 7.38 (d, J=2.0 Hz, 2H), 7.25 (t, J=2.0 Hz, 1H), 4.07-4.02 (m, 1H), 3.91-3.88 (m, 1H), 3.55-3.51 (m, 1H), 3.09-3.05 (m, 1H), 2.37-2.30 (m, 1H), 2.09-2.05 (m, 1H), 1.92-1.85 (m, 1H), 1.84-1.77 (m, 1H), 1.70-1.62 (m, 1H); LCMS: 84.6%, m/z [M+H]⁺=542.9. Regiochemistry is unknown.

234b: ¹H NMR (500 MHz, DMSO-d₆): 10.93 (br s, 1H), 10.22 (br s, 1H), 10.13 (s, 1H), 8.71 (s, 1H), 7.68 (d, J=1.5 Hz, 2H), 7.60 (s, 1H), 7.49 (d, J=1.5 Hz, 1H), 7.27 (t, J=2.0 Hz, 1H), 4.16-4.12 (m, 1H), 4.02-3.99 (m, 1H), 3.16-3.11 (m, 1H), 2.27-2.23 (m, 1H), 2.02-1.99 (m, 1H), 1.92-1.85 (m, 1H), 1.81-1.65 (m, 3H); LCMS: 85.1%, m/z [M+H]⁺=542.9. Regiochemistry is unknown.

### Example 235

### Synthesis of 5,7-dichloro-N1′-(3,5-dichlorophenyl)-N2′-hydroxy-N2′-methyl-2-oxo-1′,2′,5′,6′,7′,7a′-hexahydrospiro[indoline-3,3′-pyrrolizine]-1′,2′-dicarboxamide

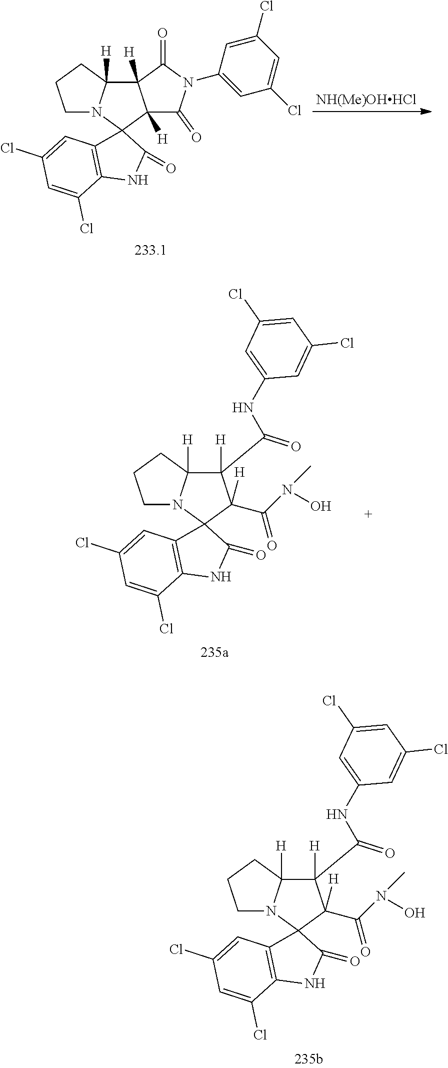 [SMILES: O=C1[C@H]2[C@H]3CCCN3C3(C(=O)Nc4c(Cl)cc(Cl)cc43)[C@H]2C(=O)N1c1cc(Cl)cc(Cl)c1 ; CN(O)C(=O)C1C(C(=O)Nc2cc(Cl)cc(Cl)c2)C2CCCN2C12C(=O)Nc1c(Cl)cc(Cl)cc12 ; "+" ; "NH(Me)OH·HCl" ; CN(O)C(=O)C1C(C(=O)Nc2cc(Cl)cc(Cl)c2)C2CCCN2C12C(=O)Nc1c(Cl)cc(Cl)cc12]

To a stirred solution of 233.1 (200 mg, 0.39 mmol) in DMF (10 mL) were added NEt₃ (0.27 mL, 1.96 mmol) and N-methylhydroxylamine hydrochloride (164 mg, 1.96 mmol) at RT. After stirring for 16 h at RT, the reaction mixture was poured into ice cold water (15 mL) and stirred for 15 minutes. The resulting precipitate was filtered, washed with water (10 mL) and dried under high vacuum. The residue was purified by prep. HPLC [Column: KROMASIL-C18 (150×25) mm, 10 μ; A: 0.1% Formic acid in H₂O, B: Acetonitrile; Gradient: (T%B): −0/30, 8/80, 10/90, 10.1/98, 12/98, 12.1/30, 14/30 at 25 mL/min] to obtain 235a (15 mg, 7%) as a white solid and 235b (30 mg, 14%) as a white solid.

235a: ¹H NMR (400 MHz, DMSO-d₆): 11.01 (br s, 1H), 9.85 (br s, 2H), 7.48 (d, J=2.0 Hz, 1H), 7.42 (d, J=2.0 Hz, 2H), 7.25-7.24 (m, 2H), 4.54-4.50 (m, 1H), 4.27-4.18 (m, 1H), 3.35-3.25 (m, 2H), 3.08 (s, 3H), 2.37-2.33 (m, 1H), 2.12-2.05 (m, 1H), 1.92-1.83 (m, 2H), 1.69-1.60 (m, 1H); LCMS: 86.30%, m/z [M+H]⁺=557.0. Regiochemistry is unknown.

235b:¹HNMR (400 MHz, DMSO-d₆): 10.67 (s, 1H), 10.20 (s, 1H), 9.72 (s, 1H), 7.78 (d, J=1.6 Hz, 1H), 7.69 (d, J=2.0 Hz, 2H), 7.41 (d, J=2.0 Hz, 1H), 7.26 (t, J=2.0 Hz, 1H), 4.26 (d, J=7.6 Hz, 1H), 3.96-3.94 (m, 1H), 3.35-3.30 (m, 1H), 2.82-2.80 (m, 1H), 2.69 (s, 3H), 2.17-2.09 (m, 1H), 1.88-1.77 (m, 3H), 1.58-1.48 (m, 1H); LCMS: 99.7%, m/z [M+H]⁺=557.0. Regiochemistry is unknown.

### Example 236

### Synthesis of (1′R,2′S,7a′R)-5,7-dichloro-N-(3,5-dichlorophenyl)-2′-(hydrazinecarbonyl)-2-oxo-1′,2′,5′,6′,7′,7a′-hexahydrospiro[indoline-3,3′-pyrrolizine]-1′-carboxamide

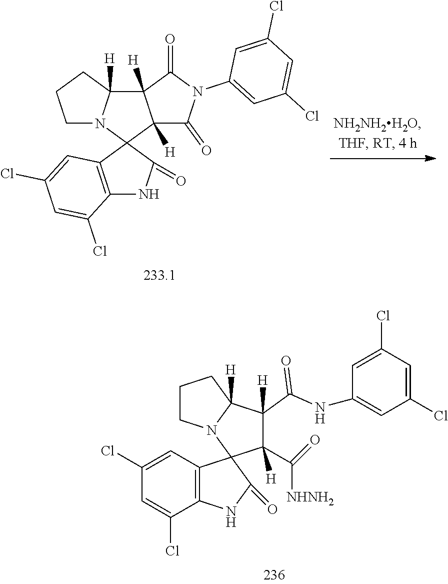 [SMILES: O=C1[C@H]2[C@H]3CCCN3C3(C(=O)Nc4c(Cl)cc(Cl)cc43)[C@H]2C(=O)N1c1cc(Cl)cc(Cl)c1 ; NNC(=O)[C@H]1[C@@H](C(=O)Nc2cc(Cl)cc(Cl)c2)[C@H]2CCCN2C12C(=O)Nc1c(Cl)cc(Cl)cc12]

To a stirred solution of 233.1 (500 mg, 0.97 mmol) in THF (5 mL) was added hydrazine mono hydrate (98 mg, 1.85 mmol) at RT. After stirring at RT for 4 h, the reaction mixture was quenched with water and extracted with EtOAc (2×10 mL). The combined organic layer was washed with brine (10 mL), dried over Na₂SO₄, filtered and concentrated under reduced pressure. The residue was triturated with CH₂Cl₂ (5 mL) to get 236 (320 mg, 60%) as a white solid.

¹H NMR (400 MHz, DMSO-d₆): 10.92 (s, 1H), 10.13 (s, 1H), 8.81 (s, 1H), 7.68 (d, J=1.6 Hz, 2H), 7.52 (s, 1H), 7.48 (s, 1H), 7.26 (s, 1H), 4.19-4.13 (m, 1H), 4.08-4.06 (m, 3H), 3.40 (d, J=7.6 Hz, 1H), 3.16-3.10 (m, 1H), 2.30-2.26 (m, 1H), 1.99-1.88 (m, 2H), 1.78-1.68 (m, 2H); LCMS: 92.3%, m/z [M+H]⁺=542.3; Chiral purity: 98.7%. Regiochemistry is unknown.

### Example 237

### Synthesis of (1′R,2′S,7a′R)-5,7-dichloro-N1′-(3,5-dichlorophenyl)-N2′-(1-methyl-1H-imidazol-4-yl)-2-oxo-1′,2′,5′,6′,7′,7a′-hexahydrospiro[indoline-3,3′-pyrrolizine]-1′,2′-dicarboxamide

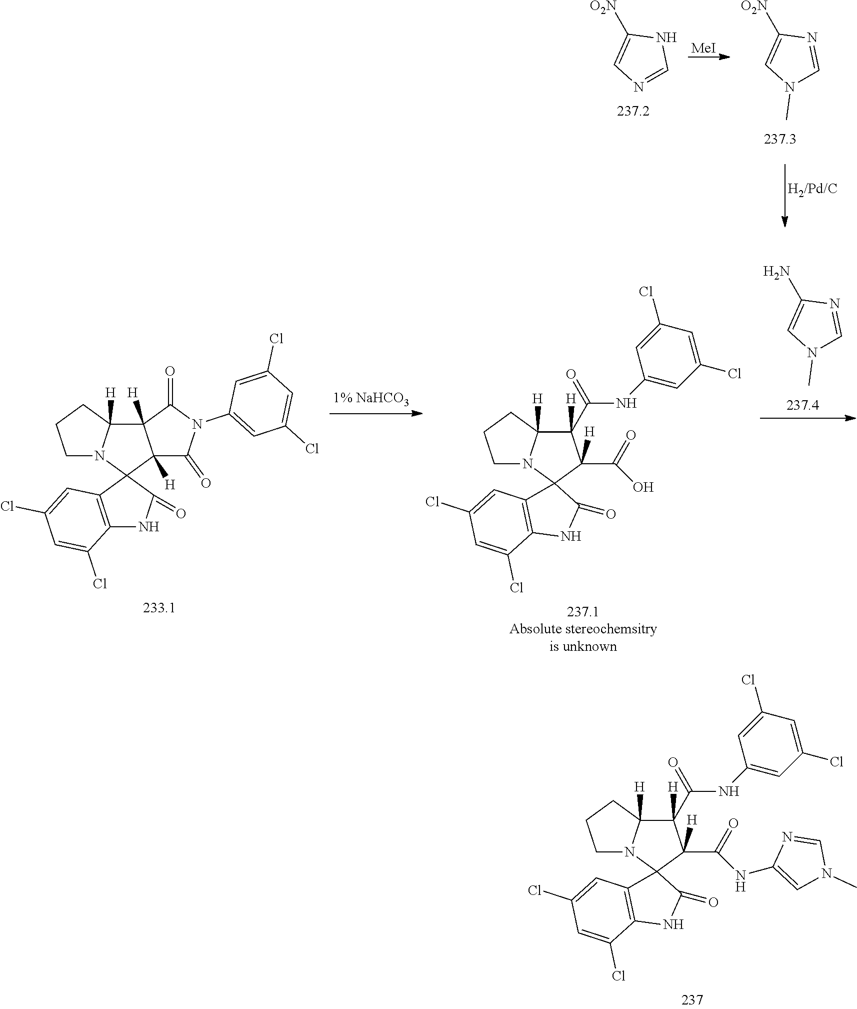

### Synthesis of 237.3

To a stirred solution of 237.2 (1 g, 8.84 mmol) in DMF (20 mL) was added NaH (0.7 g, 17.7 mmol) portion wise at 0° C. over a period of 10 minutes. After stirring at 0° C. for 1 h, MeI (1.5 g, 10.6 mmol) was added. After stirring at rt for 16 h, the reaction mixture was quenched with ice cold water and extracted with EtOAc (3×200 mL). The combined organic layer was washed with brine, dried over anhydrous Na₂SO₄, filtered and concentrated under reduced pressure to afford 237.3 (300 mg, 27%) as a gum, which was carried to next step without purification. LCMS: 95.7%, m/z [M+H]⁺=128.1.

### Synthesis of 237.4

To a stirred solution of 237.3 (1 g, 7.86 mmol) in EtOH (20 mL) was added 10% Pd/C (0.2 g) at RT. After hydrogenating at rt for 16 h using balloon pressure, the reaction mixture was filtered through Celite pad. The filtrate was concentrated under reduced pressure to afford 237.4 (800 mg) as an off-white solid. LCMS: 40.9%, m/z [M+H]⁺=98.1.

### Synthesis of 237

To a stirred solution of 237.1 (0.3 g, 0.57 mmol) in THF (10 mL) were added N-methyl morpholine (86 mg, 0.85 mmol) and isobutyl chloroformate (93 mg, 0.68 mmol) at 0° C. After stirring at 0° C. for 30 minutes, 237.4 (69 mg, 0.68 mmol) was added. After stirring for 16 h at 60° C., the reaction mixture was diluted with water and extracted with EtOAc (3×60 mL). The combined organic layer was washed with brine and dried over anhydrous Na₂SO₄ and concentrated. The residue was purified by column chromatography (Silica gel 100-200 mesh, 3% MeOH in DCM) to obtain 237 (18 mg, 5%) as a brown solid.

¹H NMR (400 MHz, DMSO-d₆): 10.94 (s, 1H), 10.07 (s, 1H), 9.94 (s, 1H), 7.64 (br s, 2H), 7.45 (s, 1H), 7.30-7.20 (m, 4H), 4.34-4.22 (m, 2H), 3.62-3.57 (m, 1H), 3.61 (s, 3H), 3.26-3.20 (m, 1H), 2.35-2.33 (m, 1H), 2.18-2.14 (m, 1H), 1.90-1.80 (m, 2H), 1.72-1.62 (m, 1H); LCMS: 95.2%, m/z [M+H]⁺=607.0. Absolute stereochemistry is unknown.

### Example 238

### Synthesis of (1′R,2′S,7a′R)-5,7-dichloro-N1′-(3,5-dichlorophenyl)-N2′-(1-methyl-1H-imidazol-2-yl)-2-oxo-1′,2′,5′,6′,7′,7a′-hexahydrospiro[indoline-3,3′-pyrrolizine]-1′,2′-dicarboxamide

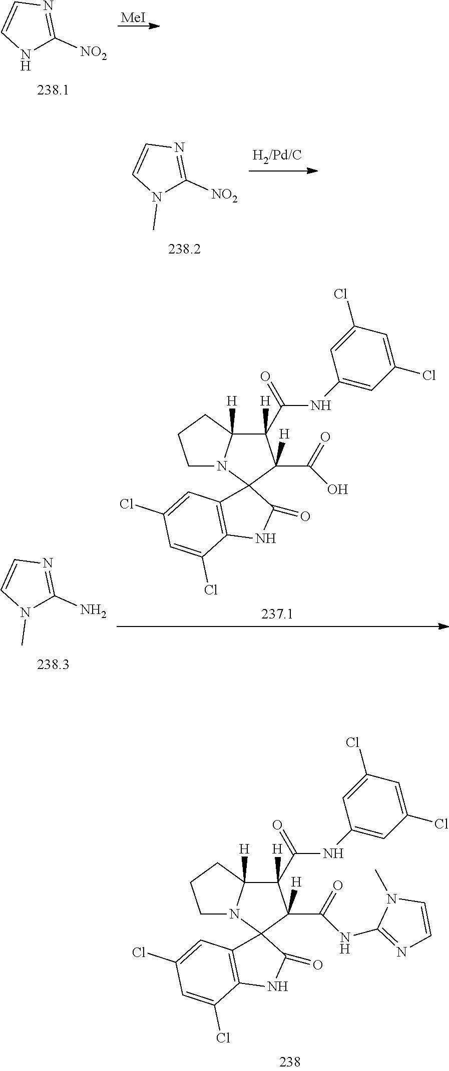

### Synthesis of 238.2

To a stirred solution of 238.1 (0.2 g, 1.76 mmol) in DMF (10 mL) was added 60% NaH (0.14 g, 3.54 mmol) portion wise at 0° C. After stirring for 1 h, MeI (0.25 g, 1.77 mmol) was added at 0° C. After stirring at RT for 16 h, the reaction mixture was quenched with ice cold water and extracted with EtOAc (3×50mL). The combined organic layer was washed with brine and dried over anhydrous Na₂SO₄ and concentrated to obtain 238.2 (0.14 g) as a gummy material.

¹H NMR (500 MHz, CDCl₃): 7.14 (d, J=1.0 Hz, 1H), 7.06 (s, 1H), 4.08 (s, 3H); LCMS: 57.04%, m/z [M+H]⁺=127.9.

### Synthesis of 238.3

To a stirred solution of 238.2 (0.1 g, 0.79 mmol) in 1,4-dioxane (5 mL) was added 10% Pd/C (50% wet, 20 mg). After hydrogenating for16 hat RT using balloon pressure, the reaction mixture was filtered through Celite pad. The filtrate was concentrated under reduced pressure to obtain 238.3 (70 mg) as an off-white solid. LCMS: 98.2%, m/z [M+H]⁺=98.2.

### Synthesis of 238

To a stirred solution of 237.1 (0.25 g, 0.47 mmol) in THF (10 mL) were added N-methyl morpholine (72 mg, 0.71 mmol) and isobutyl chloroformate (77 mg, 0.57 mmol) at 0° C. After stirring at 0° C. for 30 minutes, 238.3 (69 mg, 0.71 mmol) was added. After stirring for 24 h at 60° C., the reaction mixture was diluted with water and extracted with EtOAc (3×50 mL). The combined organic layer was washed with brine and dried over anhydrous Na₂SO₄ and concentrated. The residue was purified by column chromatography (Silica gel 100-200 mesh, 3% MeOH in DCM) followed by prep. HPLC [Column: KROMASIL-C18 (150×25) mm, 10 μ; A: 0.1% Formic acid in H₂O, B: Acetonitrile; Gradient: (T%B): −0/30, 7/70, 7.1/98, 9/98, 9.1/30, 11/30 at 25 mL/min] to obtain 238 (11 mg, 4%) as a pale pink solid.

¹H NMR (400 MHz, DMSO-d₆): 11.82 (br s, 1H), 10.61 (s, 1H), 10.54 (s, 1H), 7.76 (s, 2H), 7.45 (s, 2H), 7.25 (s, 1H), 6.76 (s, 1H), 6.60 (s, 1H), 4.21-4.11 (m, 1H), 4.06 (d, J=3.2 Hz, 2H), 2.96 (s, 3H), 2.91-2.83 (m, 1H), 2.50-2.40 (m, 1H), 1.81-1.72 (m, 2H), 1.61-1.48 (m, 2H); LCMS: 96.2%, m/z [M−H]⁻=605.2. Absolute stereochemistry is unknown.

### Example 239

### Synthesis of (1′R,2′ S,7a′R)-5,7-dichloro-N1′-(3,5-dichlorophenyl)-N2′-(1-methyl-1H-pyrazol-5-yl)-2-oxo-1′,2′,5′,6′,7′,7a′-hexahydrospiro[indoline-3,3′-pyrrolizine]-1′,2′-dicarboxamide

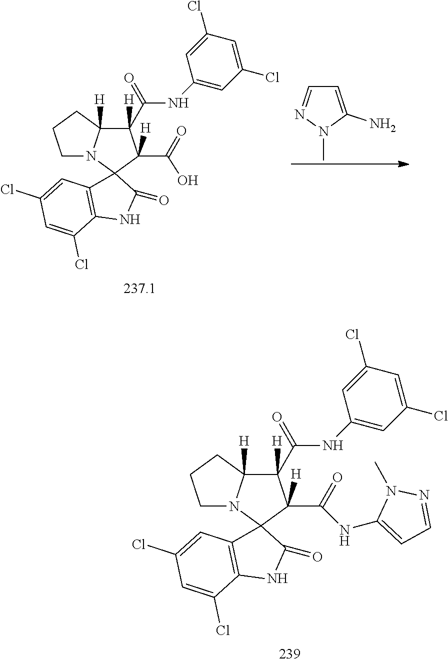

N-Methyl morpholine (151 mg, 1.50 mmol) was added to 237.1 (400 mg, 0.75 mmol) in THF (40 mL) at −10° C. followed by isobutyl chloroformate (204 mg, 1.50 mmol). After stirring for 20 minutes at −10° C., 1-methyl-1H-pyrazol-5-amine (220 mg, 2.26 mmol) was added and stirred for 1 h at the same temperature. The reaction mixture was concentrated under reduced pressure to obtain residue which was purified by reverse phase chromatography [Column: Buchi Reveleris C₁₈ (40 g); B: 0.05% Formic acid in H₂O, B: Acetonitrile]. Pure fractions were lyophilized to get 239 (40 mg, 8%) as an off-white solid.

¹H NMR (400 MHz, DMSO-d₆): 11.07 (s, 1H), 10.16 (s, 1H), 9.63 (s, 1H), 7.66 (d, J=1.6 Hz, 2H), 7.56 (d, J=2.0 Hz, 1H), 7.35 (d, J=2.0 Hz, 1H), 7.31 (d, J=1.6 Hz, 1H), 7.27 (t, J=2.0 Hz, 1H), 5.90 (d, J=1.6 Hz, 1H), 4.31-4.26 (m, 2H), 3.63 (d, J=7.2 Hz, 1H), 3.51 (s, 3H), 3.20-3.18 (m, 1H), 2.36-2.33 (m, 1H), 2.12-2.02 (m, 1H), 1.92-1.79 (m, 2H), 1.71-1.63 (m, 1H); LCMS: 96.9%, m/z [M+H]⁺=607.0. Absolute stereochemistry is unknown.

### Example 240

### Synthesis of (1′R,2′S,7a′R)-5,7-dichloro-N1′-(3,5-dichlorophenyl)-N2′-(1-methyl-1H-pyrazol-3-yl)-2-oxo-1′,2′,5′,6′,7′,7a′-hexahydrospiro[indoline-3,3′-pyrrolizine]-1′,2′-dicarboxamide

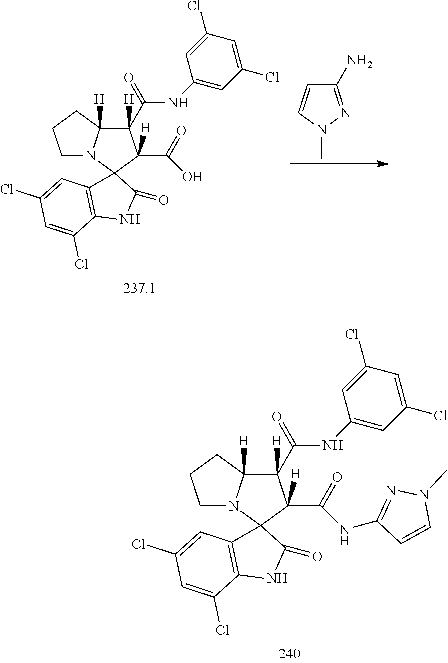

To a stirred solution of 237.1 (100 mg, 0.18 mmol) in THF (10 mL) were added at 0° C. N-methyl morpholine (38 mg, 0.37 mmol) and isobutyl chloroformate (51 mg, 0.37 mmol). After stirring for 15 minutes, 1-methyl-1H-pyrazol-3-amine (36 mg, 0.37 mmol) was added. After stirring for 1 h at 0° C., the reaction mixture was concentrated under reduced pressure. The residue was purified by reverse phase chromatography [Column: Buchi Reveleris C18 (40 g); B: 0.05% Formic acid in H₂O, B: Acetonitrile] to afford 240 (20 mg, 17%) as an off-white solid.

¹H NMR (400 MHz, DMSO-d₆): 10.97 (s, 1H), 10.10 (s, 1H), 10.05 (s, 1H), 7.65 (d, J=2.0 Hz, 2H), 7.50 (d, J=2.0 Hz, 1H), 7.47 (d, J=2.0 Hz, 1H), 7.33 (d, J=2.0 Hz, 1H), 7.25 (t, J=2.0 Hz, 1H), 6.38 (d, J=2.0 Hz, 1H), 4.35-4.25 (m, 2H), 3.70 (s, 3H), 3.57 (d, J=8.0 Hz, 1H), 3.26-3.20 (m, 1H), 2.36-2.32 (m, 1H), 2.15-2.09 (m, 1H), 1.92-1.88 (m, 1H), 1.83-1.80 (m, 1H), 1.68-1.66 (m, 1H); LCMS: 93.1%, m/z [M+H]⁺=607.0. Absolute stereochemistry is unknown.

### Example 241

### Synthesis of (1′R,2′ S,7a′R)-5,7-dichloro-N1′-(3,5-dichlorophenyl)-N2′-methoxy-N2′-methyl-2-oxo-1′,2′,5′,6′,7′,7a′-hexahydrospiro[indoline-3,3′-pyrrolizine]-1′,2′-dicarboxamide

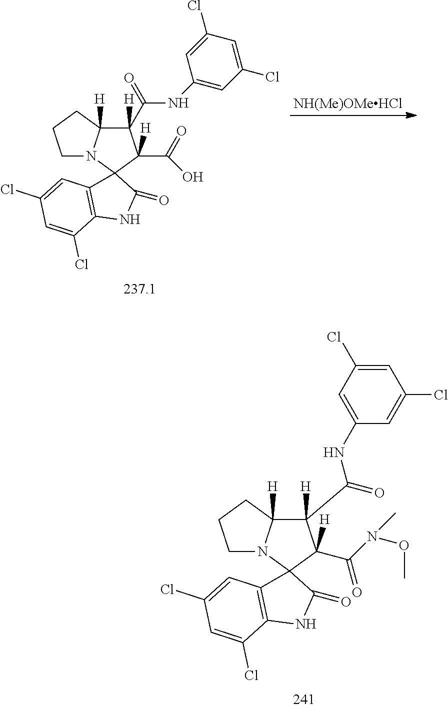

To a stirred solution of 237.1 (300 mg, 0.56 mmol) in DMF (10 mL) were added N-methylmorpholine (0.09 mL, 0.85 mmol) and isobutyl chloroformate (0.1 mL, 0.73 mmol) at 0° C. After 15 minutes, N,O-dimethylhydroxylamine hydrochloride (276 mg, 2.83 mmol) was added at 0° C. After stirring for 16 h at RT, the reaction mixture was poured into ice cold water (20 mL) and stirred for 20 minutes. The resulting precipitate was filtered. The solid was collected, washed with water (20 mL) and dried under high vacuum. The residue was purified by prep. HPLC [Column: KROMASIL-C18 (150×25) mm, 10 μ; A: 0.1% Formic acid in H₂O, B: Acetonitrile; Gradient: (T%B): −0/50, 8/80, 10/80, 10.1/98, 11/98, 11.1/50, 13/50 at 25 mL/min] to obtain 241 (17 mg, 5% yield) as a white solid.

¹H NMR (400 MHz, DMSO-d₆): 10.72 (br s, 1H), 10.24 (br s, 1H), 7.81 (d, J=2.0 Hz, 1H), 7.70 (d, J=1.6 Hz, 2H), 7.44 (d, J=2.0 Hz, 1H), 7.27 (t, J=2.0 Hz, 1H), 4.25 (d, J=7.2 Hz, 1H), 4.01-3.95 (m, 1H), 3.43 (s, 3H), 3.37-3.35 (m, 1H), 2.90-2.82 (m, 1H), 2.66 (s, 3H), 2.17-2.10 (m, 1H), 1.90-1.72 (m, 3H), 1.61-1.53 (m, 1H); LCMS: 95.2%, m/z [M+H]⁺=571.0. Absolute stereochemistry is unknown.

### Example 242

### Synthesis of (1′R,2′S,7a′R)-2′-(2-acetylhydrazine-1-carbonyl)-5,7-dichloro-N-(3,5-dichlorophenyl)-2-oxo-1′,2′,5′,6′,7′,7a′-hexahydrospiro[indoline-3,3′-pyrrolizine]-1-carboxamide

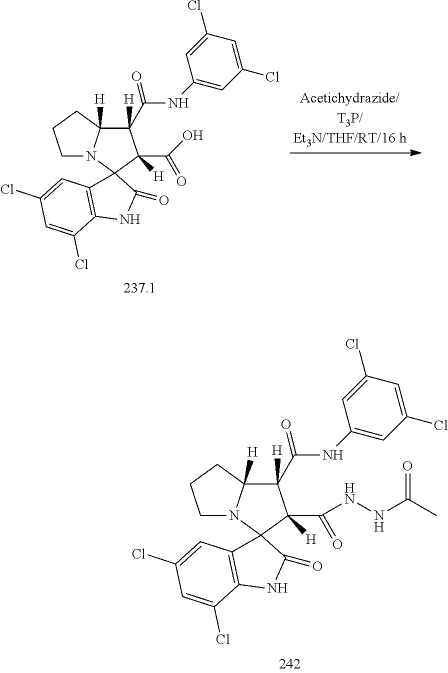 [SMILES: O=C(Nc1cc(Cl)cc(Cl)c1)[C@H]1[C@H]2CCCN2C2(C(=O)Nc3c(Cl)cc(Cl)cc32)[C@H]1C(=O)O ; CC(=O)NNC(=O)[C@H]1[C@@H](C(=O)Nc2cc(Cl)cc(Cl)c2)[C@H]2CCCN2C12C(=O)Nc1c(Cl)cc(Cl)cc12]

To a stirred solution of 237.1 (100 mg, 0.18 mmol) in THF (3 mL) were added triethyl amine (0.1 mL, 0.75 mmol) and acetichydrazide (28 mg, 0.37 mmol) at RT. After 10 minutes, propylphosphonic anhydride solution (50% wt in EtOAc, 0.24 mL, 0.75 mmol) was added at RT. After stirring for 16 h at RT, the reaction mixture was concentrated under reduced. The residue was purified by prep. HPLC [Column: KROMASIL-C18 (150×25) mm, 10 μ; A: 0.1% Formic acid in H₂O, B: Acetonitrile; Gradient: (T%B): −0/40, 8/90, 8.1/98, 10/98, 10.1/40, 12/40 at 25 mL/min] to afford 242 (27 mg, 24%) as a white solid.

¹H NMR (400 MHz, DMSO-d₆): 10.89 (s, 1H), 10.22 (s, 1H), 9.83 (s, 1H), 9.71 (s, 1H), 7.71-7.68 (m, 2H), 7.59 (d, J=2.0 Hz, 1H), 7.42 (d, J=2.0 Hz, 1H), 7.27 (t, J=2.0 Hz, 1H), 4.18-4.12 (m, 1H), 4.08-4.04 (m, 1H), 3.68 (d, J=8.0 Hz, 1H), 2.96-2.90 (m, 1H), 2.28-2.24 (m, 1H), 1.88-1.68 (m, 4H), 1.77 (s, 3H); LCMS: 95.6%, m/z [M+H]⁺=584.0. Absolute stereochemistry is unknown.

### Example 243

### Synthesis of (1′R,2′S,7a′R)-5,7-dichloro-N1′-(3,5-dichlorophenyl)-N2′-morpholino-2-oxo-1′,2′,5′,6′,7′,7a′-hexahydrospiro dicarboxamide

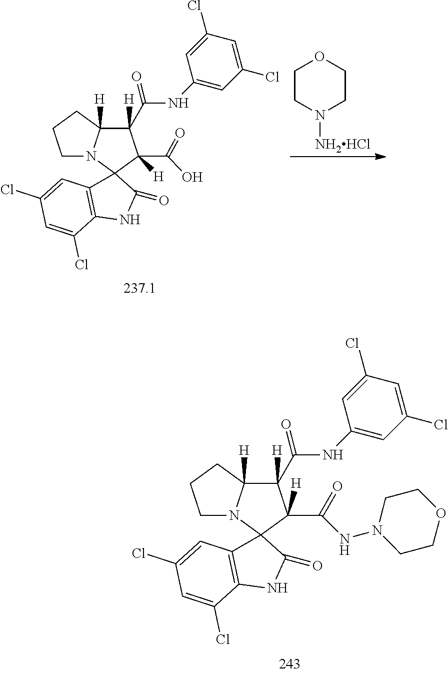

To a stirred solution of 237.1 (200 mg, 0.37 mmol) in DMF (10 mL) were added at 0° C. N-methyl morpholine (0.05 mL, 0.49 mmol) and isobutyl chloroformate (0.07 mL, 0.56 mmol). After 10 mintues, morpholin-4-amine hydrochloride (0.07 mL, 0.56 mmol) was added at 0° C. After stirring for 2 h at RT, the reaction mixture was poured into ice cold water (15 mL) and stirred for 15 minutes. The resulting precipitate was filtered, washed with water (10 mL) and dried under high vacuum. The compound material was purified by prep. HPLC [Column: KROMASIL-C18 (150×25) mm, 10 μ; A: 10 mM NH₄OAc in H₂O, B: Acetonitrile; Gradient: (T%B): −0/50, 8/80, 9/80, 9.1/50, 11/50 at 25 mL/min] to obtain 243 (35 mg, 15%) as a white solid.

¹H NMR (400 MHz, DMSO-d₆): 10.81 (br s, 1H), 10.15 (br s, 1H), 8.18 (s, 1H), 7.90 (d, J=1.6 Hz, 1H), 7.62 (d, J=1.2 Hz, 2H), 7.37 (d, J=1.6 Hz, 1H), 7.18 (s, 1H), 4.12 (d, J=7.2 Hz, 1H), 3.79-3.74 (m, 1H), 3.57-3.47 (m, 2H), 3.39-3.25 (m, 3H), 2.71-2.60 (m, 2H), 2.26-2.10 (m, 2H), 2.02-2.00 (m, 1H), 1.75-1.69 (m, 3H), 1.41-1.37 (m, 1H), 1.02-1.00 (m, 1H); LCMS: 99.6%, m/z [M+H]⁺=612.0. Absolute stereochemistry is unknown.

### Example 244

### Synthesis of (1′R,2′S,6′S,7a′R)-5,7-dichloro-N1′-(3,5-dichlorophenyl)-6′-hydroxy-6′-methyl-2-oxo-1′,2′,5′,6′,7′,7a′-hexahydrospiro[indoline-3,3′-pyrrolizine]-1,2′-dicarboxamide

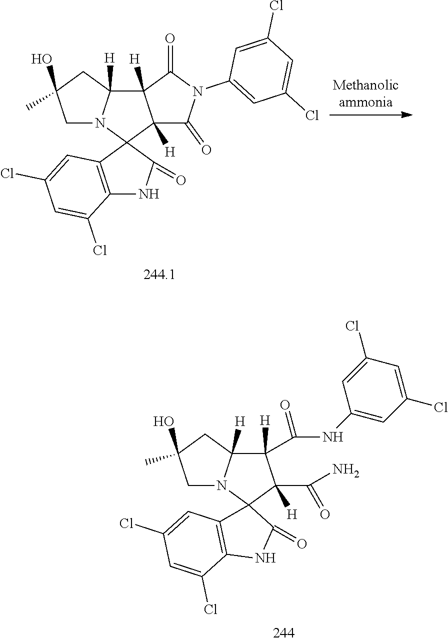 [SMILES: C[C@]1(O)C[C@@H]2[C@@H]3C(=O)N(c4cc(Cl)cc(Cl)c4)C(=O)[C@@H]3C3(C(=O)Nc4c(Cl)cc(Cl)cc43)N2C1 ; C[C@]1(O)C[C@@H]2[C@H](C(=O)Nc3cc(Cl)cc(Cl)c3)[C@H](C(N)=O)C3(C(=O)Nc4c(Cl)cc(Cl)cc43)N2C1]

Methanolic ammonia (10 mL) was added to 244.1 (WO2017117239) (250 mg, 0.46 mmol) at RT. After stirring for 16 h, the reaction mixture was concentrated under reduced pressure. The residue was purified by prep. HPLC [Column: YMC TRIART-C₁₈ (150×25) mm, 10 μ; A: 0.1% Formic acid in H₂O, B: Acetonitrile; Gradient: (T%B): −0/20, 8/60, 10/60, 10.1/20, 12/20 at 25 mL/min] to obtain 244 (32 mg, 12%) as a white solid.

¹H NMR (500 MHz, DMSO-d₆): 10.74 (s, 1H), 10.61 (s, 1H), 7.71 (d, J=1.5 Hz, 2H), 7.65 (d, J=1.5 Hz, 1H), 7.48 (d, J=2.0 Hz, 1H), 7.30 (t, J=2.0 Hz, 1H), 6.91 (s, 1H), 6.57 (s, 1H), 4.50-4.47 (m, 1H), 4.43 (s, 1H), 4.32-4.28 (m, 1H), 3.81 (d, J=11.5 Hz, 1H), 2.92 (d, J=8.5 Hz, 1H), 2.30 (d, J=8.0 Hz, 1H), 1.69-1.65 (m, 1H), 1.50-1.45 (m, 1H), 1.21 (s, 3H); LCMS: 99.5%, m/z [M+H]⁺=557.0; Chiral Purity: 99.9%. Regiochemistry is unknown.

### Example 245

### Synthesis of (1′R,2′S,6′S,7a′R)-5,7-dichloro-N1′-(3,5-dichlorophenyl)-N2′,6′-dihydroxy-6′-methyl-2-oxo-1′,2′,5′,6′,7′,7a′-hexahydrospiro[indoline-3,3′-pyrrolizine]-1′,2′-dicarboxamide

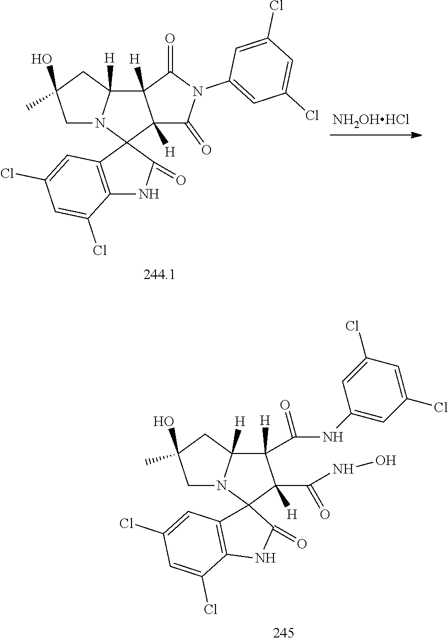

To a stirred solution of 244.1 (500 mg, 0.92 mmol) in DMF (15 mL) was added NH₂OH.HCl (320 mg, 4.61 mmol)) and Et₃N (0.64 mL, 4.61 mmol) at RT and the resulting reaction mixture was stirred for 16 h. The reaction mixture was diluted with ice cold water (20 mL) and the resulting precipitate was filtered to obtain the residue which was purified by prep. HPLC [Column: KROMASIL-C18 (150×25) mm, 10 μ; A: 0.1% Formic acid in H₂O, B: Acetonitrile; Gradient: (T%B): −0/20, 8/60, 8/98, 10/98, 10.1/20, 13/20 at 25 mL/min] to obtain 245 (30 mg, 5%) as an off-white solid.

¹H NMR (400 MHz, DMSO-d₆): 10.91 (br s, 1H), 10.21 (br s, 1H), 10.08 (br s, 1H), 8.75 (br s, 1H), 7.67 (d, J=2.0 Hz, 2H), 7.50 (s, 1H), 7.47 (s, 1H), 7.25 (t, J=1.6 Hz, 1H), 4.51-4.42 (m, 1H), 4.00 (s, 1H), 4.22-4.18 (m, 1H), 3.23 (d, J=9.2 Hz, 1H), 3.17 (d, J=7.6 Hz, 1H), 2.20-2.14 (m, 2H), 1.69-1.65 (m, 1H), 1.20 (s, 3H); LCMS: 90.5%, m/z [M+H]⁺=573.0. Regiochemistry is unknown.

### Example 246

### Synthesis of (1′R,2′S,6′S,7a′R)-5,7-dichloro-N1′-(3,5-dichlorophenyl)-6′-hydroxy-N2′-methoxy-6′-methyl-2-oxo-1′,2′,5′,6′,7′,7a′-hexahydrospiro[indoline-3,3′-pyrrolizine]-1′,2′-dicarboxamide

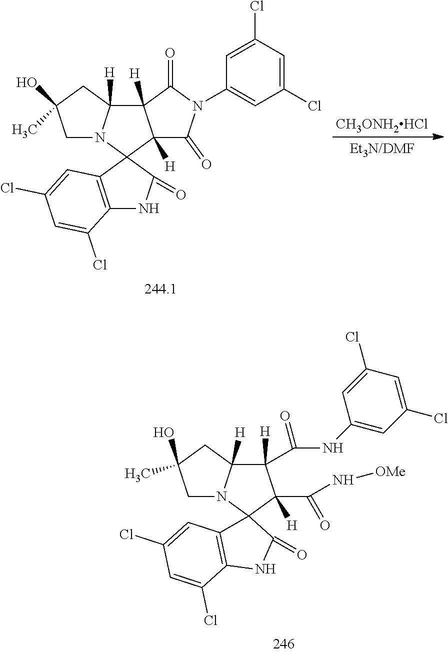

To a stirred solution of 244.1 (300 mg, 0.55 mmol) in DMF (15 mL) were added Et₃N (1.1 mL, 8.31 mmol) and CH₃ONH_{2.}HCl (462 mg, 5.54 mmol) at RT. After stirring at RT for 48 h, the reaction mixture was diluted with water (50 mL) and extracted with EtOAc (2×30 mL). The combined organic layer was dried over Na₂SO₄, filtered and evaporated by nitrogen bubbling. The resulting residue was purified by prep. HPLC [Column: KROMASIL-C18 (150×25) mm, 10 μ; A: 0.1% Formic acid in H₂O, B: Acetonitrile; Gradient: (T%B): −0/40, 7/65, 7.1/98, 9/98, 9.1/40, 11/40 at 25 mL/min] to obtain 246 (25 mg, 12%) as an off-white solid.

¹H NMR (500 MHz, DMSO-d₆): 11.00 (s, 1H), 10.80 (s, 1H), 10.13 (s, 1H), 7.66 (d, J=1.5 Hz, 2H), 7.56 (s, 1H), 7.42 (s, 1H), 7.26 (d, J=2.0 Hz, 1H), 4.57-4.48 (m, 1H), 4.43 (s, 1H), 4.27-4.23 (m, 1H), 3.45 (s, 3H), 3.32-3.24 (m, 1H), 3.01 (d, J=8.0 Hz, 1H), 2.24-2.14 (m, 2H), 1.68-1.61 (m, 1H); LCMS: 96.4%, m/z [M+H]⁺=587.0. Regiochemistry is unknown.

### Example 247

### Synthesis of (6′S)-5,7-dichloro-N1′-(3,5-dichlorophenyl)-6′-hydroxy-6′-methyl-N2′-(methylsulfonyl)-2-oxo-1′,2′,5′,6′,7′,7a′-hexahydrospiro[indoline-3,3′-pyrrolizine]-1′,2′-dicarboxamide

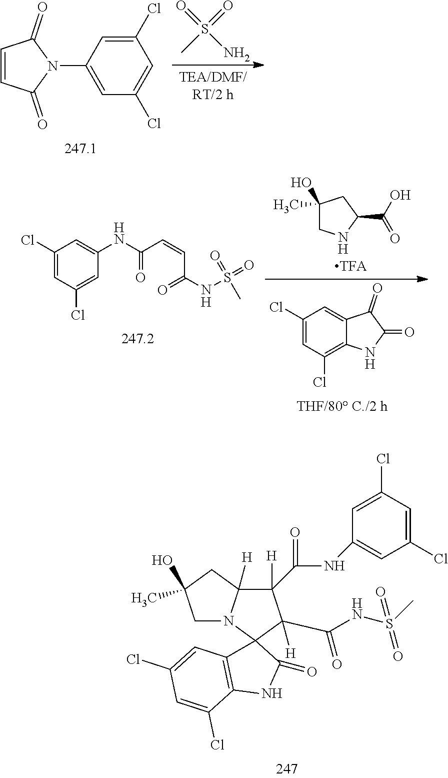

### Synthesis of 247.2

To a stirred solution of 247.1 (1 g, 4.13 mmol) in DMF (5 mL) were added methanesulfonamide (393 mg, 4.13 mmol) and TEA (1.72 mL, 12.4 mmol) at RT. After stirring at RT for 2 h, the reaction mixture was concentrated under reduced pressure to obtain 247.2 (1.39 g) as a thick brown liquid, which used in the next step without any purification. LCMS: 57.2%, m/z [M−H]⁻=334.7.

### Synthesis of 247

To a stirred solution of 247.2 (1.2 g, 3.55 mmol) in THF (20 mL) were added TFA salt of (2S,4S)-4-hydroxy-4-methyl-1-(2,2,2-trifluoroacetyl)-114-pyrrolidine-2-carboxylic acid (860 mg, 3.55 mmol) and 5,7-dichloroindoline-2,3-dione (767 mg, 3.55 mmol) at RT. After stirring for 2 h at 80° C., the reaction mixture was cooled to RT and diluted with EtOAc. The organic solution was collected, washed with ice cold water, dried over Na₂SO₄, filtered and concentrated using a stream of nitrogen gas. The residue was purified by prep. HPLC [Column: KROMASIL-C18 (150×25) mm, 10 μ; A: 0.1% Formic acid in H₂O, B: Acetonitrile; Gradient: (T%B): −0/45, 7/55, 7.1/98, 10/98, 10.1/45, 12/45 at 25 mL/min] to obtain 247 (26 mg) as an off-white solid. Regiochemistry was not confirmed.

¹H NMR (400 MHz, DMSO-d₆): 11.75 (br s, 1H), 11.02 (br s, 1H), 9.98 (s, 1H), 7.49 (s, 1H), 7.41 (d, J=2 Hz, 2H), 7.26 (s, 1H), 7.12 (br s, 1H), 4.56-4.42 (m, 2H), 4.28-4.19 (m, 1H), 3.39 (d, J=7.6 Hz, 1H), 3.22-3.10 (m, 4H), 2.25-2.10 (m, 2H), 1.80-1.75 (m, 1H), 1.24 (s, 3H); LCMS: 90.9%, m/z [M+H]⁺=634.9.

### Example 248

### Synthesis of (6′S)-5,7-dichloro-N1′-(3,5-dichlorophenyl)-N2′-(N,N-dimethylsulfamoyl)-6′-hydroxy-6′-methyl-2-oxo-1′,2′,5′,6′,7′,7a′-hexahydrospiro[indoline-3,3′-pyrrolizine]-1,2′-dicarboxamide

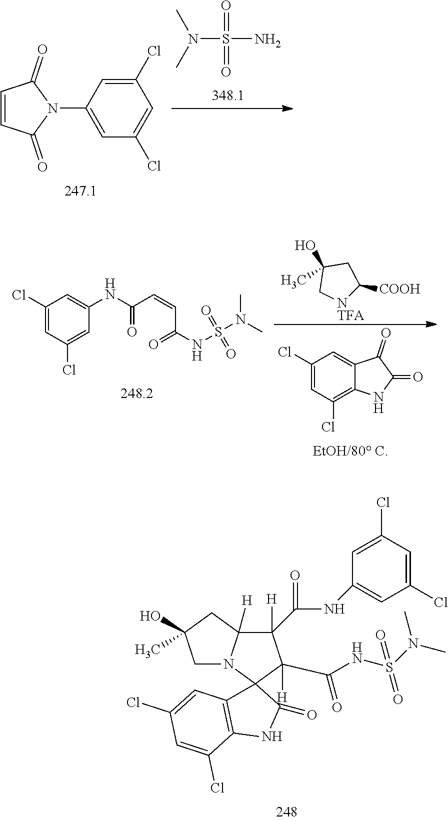

### Synthesis of 248.2

To a stirred solution of 247.1 (500 mg, 2.06 mmol) and 248.1 (256 mg, 2.06 mmol) in DMF (3 mL) was added Et₃N (0.86 mL, 6.19 mmol) at RT. After stirring at RT for 2 h, the reaction mixture was evaporated under reduced pressure to afford 248.2, which used as such in the next step. LCMS: (13+39)%, m/z [M−H]⁻=364.0.

### Synthesis of 248

To a stirred solution of 248.2 (300 mg, 0.81 mmol) in THF (10 mL) were added TFA salt of (2S,4S)-4-hydroxy-4-methyl-1-(2,2,2-trifluoroacetyl)-114-pyrrolidine-2-carboxylic acid (197 mg, 0.81 mmol) and 5,7-dichloroindoline-2,3-dione (176 mg, 0.81 mmol) at RT. After stirring at 80° C. for 2 h, the reaction mixture was cooled to RT and diluted with EtOAc (20 mL). The organic solution was collected, washed with ice cold water, dried over Na₂SO₄ filtered and concentrated under reduced pressure. The residue was purified by prep. HPLC [Column: KROMASIL-C18 (150×25) mm, 10 μ; A: 0.1% Formic acid in H₂O, B: Acetonitrile; Gradient: (T%B): −0/40, 8/70, 8.1/40, 10/40 at 25 mL/min] to afford 248 (16 mg, 3%) as an off-white solid. Regiochemistry was not confirmed. LCMS: 92.6%, m/z [M+H]⁺=664.0.

### Example 249

### Synthesis of (1′S,2′R,3S,7a′S)-5,7-dichloro-1-((3-chloro-5-methoxyphenyl)(methyl)carbamoyl)-6′,6′-difluoro-2-oxo-1′,2′,5′,6′,7′,7a′-hexahydrospiro[indoline-3,3′-pyrrolizine]-2′-carboxylic acid (249a) & (1′R,2′S,3R,7a′R)-5,7-dichloro-1-((3-chloro-5-methoxyphenyl)(methyl)carbamoyl)-6′,6′-difluoro-2-oxo-1′,2′,5′,6′,7′,7a′-hexahydrospiro[indoline-3,3′-pyrrolizine]-2′-carboxylic acid (249b):

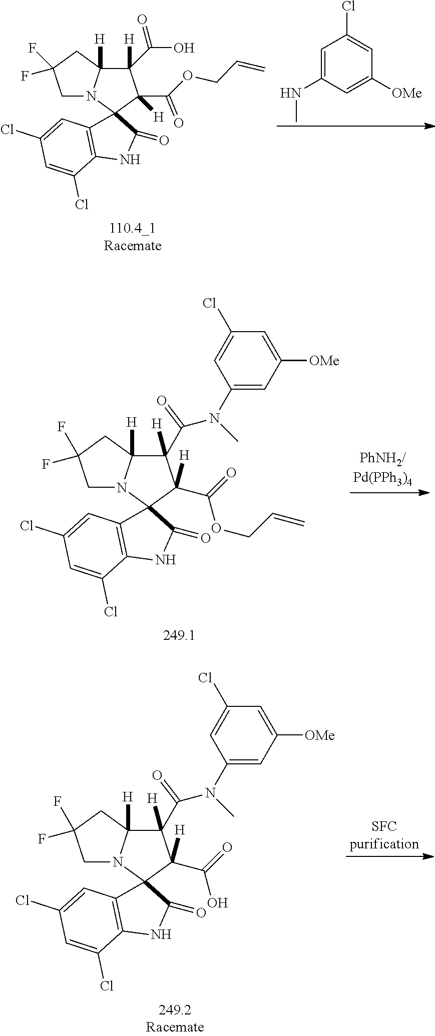

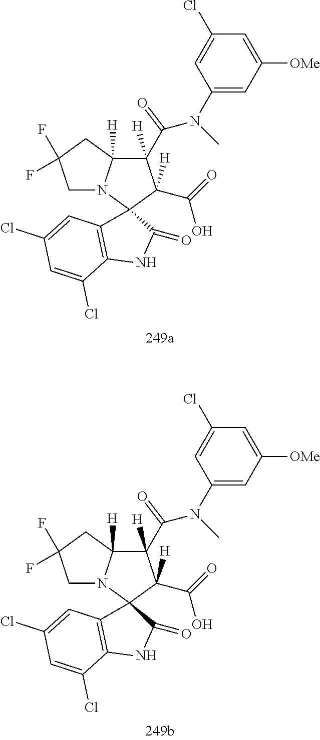 [SMILES: COc1cc(Cl)cc(N(C)C(=O)[C@H]2[C@@H](C(=O)O)[C@]3(C(=O)Nc4c(Cl)cc(Cl)cc43)N3CC(F)(F)C[C@@H]23)c1 ; COc1cc(Cl)cc(N(C)C(=O)[C@H]2[C@H]3CC(F)(F)CN3[C@]3(C(=O)Nc4c(Cl)cc(Cl)cc43)[C@H]2C(=O)O)c1]

249a and 249b were synthesized from 110.4_1 following the procedure described for the synthesis of 265a and 265b.

249a: ¹H NMR (500 MHz, DMSO-d₆) (Exist in rotameric form): 12.55 (br s, 1H), 10.97 (br s, 1H), 8.34 (s, 1H), 7.47-7.44 (m, 1H), 7.05-6.86 (m, 3H), 4.15-3.95 (m, 1H), 3.83-3.79 (m, 1H), 3.79 (s, 3H), 3.60-3.50 (m, 1H), 3.32-3.25 (m, 1H), 3.23 (s, 3H), 2.60-2.49 (m, 1H), 2.45-2.30 (m, 1H), 2.14-2.07 (m, 1H); LCMS: 93.1%, m/z [M+H]⁺=574.1.

249b:¹HNMR (500 MHz, DMSO-d₆) (Exist in rotameric form): 12.51 (br s, 1H), 11.04/10.97 (br s, 1H), 8.34 (s, 1H), 7.47-7.44 (m, 1H), 7.05-6.85 (m, 3H), 4.15-3.95 (m, 1H), 3.83-3.79 (m, 1H), 3.79/3.68 (s, 3H), 3.60-3.50 (m, 1H), 3.37-3.24 (m, 1H), 3.23 (s, 3H), 2.60-2.50 (m, 1H), 2.45-2.30 (m, 1H), 2.14-2.07 (m, 1H); LCMS: 92.2%, m/z [M+H]⁺=574.1.

### Example 250

### Synthesis of rac-(5-methyl-2-oxo-1,3-dioxo1-4-yl)methyl (1′R,2′S,3R,7a′R)-5,7-dichloro-1-((3,5-dichlorophenyl)(methyl)carbamoyl)-6′,6′-difluoro-2-oxo-1′,2′,5′,6′,7′,7a′-hexahydrospiro[indoline-3,3′-pyrrolizine]-2′-carboxylate (250.3a) & rac-(5-methyl-2-oxo-1,3-dioxo1-4-yl)methyl (1′R,2′S,3R,7a′R)-5,7-dichloro-1′-((3,5-dichlorophenyl)(methyl)carbamoyl)-6′,6′-difluoro-1-45-methyl-2-oxo-1,3-dioxol-4-yl)methyl)-2-oxo-1′,2′,5′,6′,7′,7a′-hexahydrospiro[indoline-3,3′-pyrrolizine]-2′-carboxylate (250.3b)

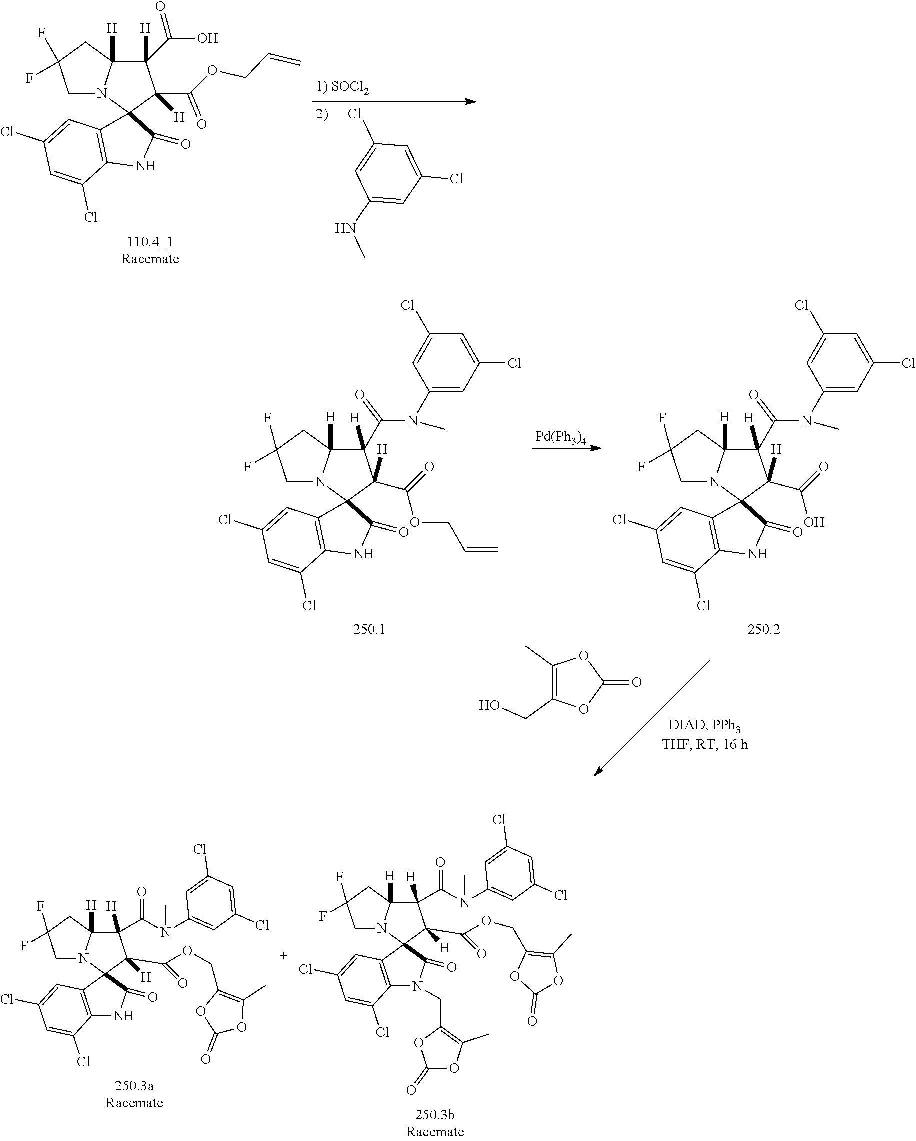

### Synthesis of 250.1

Thionyl chloride (20 mL) was added to 110.4_1 (1.5 g, 3.25 mmol) at RT. After stirring for 2 h, the excess thionyl chloride was removed under reduced pressure to give an acid chloride. To this acid chloride in CH₂Cl₂ (25 mL) was added a solution of 3,5-dichloro-N-methylaniline (1.24 g, 7.08 mmol) in CH₂Cl₂ (5 mL) at RT. After stirring for 16 h at RT, the reaction mixture was quenched with water (10 mL). The organic layer was separated, and the aqueous layer was extracted with CH₂Cl₂ (2×20 mL). The combined organic layer was washed with brine (20 mL), dried over anhydrous Na₂SO₄, filtered and concentrated under reduced pressure. The residue was purified by flash column chromatography (40 g Silica gel cartridge, 30% EtOAc in pet ether) to afford 250.1 (1.4 g, 73%) as a solid.

¹H NMR (400 MHz, DMSO-d₆) (exist in rotameric form): 11.10/11.05 (br s, 1H), 8.20/7.76 (d, J=2.0 Hz, 1H), 7.68/7.57 (t, J=2.0 Hz, 1H), 7.50-7.42 (m, 3H), 5.45-5.36 (m, 1H), 5.10-5.06 (m, 2H), 4.29-4.18 (m, 2H), 4.13 (d, J=7.6 Hz, 1H), 3.84-3.80 (m, 1H), 3.64-3.60 (m, 1H), 3.41/3.24 (s, 3H), 3.35-3.24 (m, 1H), 2.67-2.55 (m, 1H), 2.43-2.33 (m, 1H), 2.17-2.09 (m, 1H); LCMS: 95.1%, m/z [M+H]⁺=620.2.

### Synthesis of 250.2

To a stirred solution of 250.1 (1.4 g, 2.26 mmol) in THF (20 mL) were added aniline (210 mg, 2.26 mmol) and Pd(PPh₃)₄ (522 mg, 0.45 mmol) at RT. After stirring for 2 h at RT, the reaction mixture was concentrated under reduced pressure. The residue was purified by prep. HPLC [Column: X-SELECT-C18 (150×30) mm, 5 μ; A: 0.1% HCOOH in H₂O, B: Acetonitrile; Gradient: (Time/%B): 0/45, 8/80, 10/80, 10.1/98, 13/98, 13.1/45, 15/45 at 18 ml/min] to afford 250.2 (430 mg, 33%) as a solid.

¹H NMR (500 MHz, DMSO-d₆) (Exist in rotameric form):12.54 (br s, 1H), 11.07/10.99 (br s, 1H), 8.30/7.88 (d, J=2.0 Hz, 1H), 7.66-7.37 (m, 4H), 4.01 (d, J=7.5 Hz, 1H), 3.84-3.80 (m, 1H), 3.56-3.53 (m, 1H), 3.40/3.24 (s, 3H), 3.30-3.24 (m, 1H), 2.64-2.55 (m, 1H), 2.42-2.32 (m, 1H), 2.19-2.03 (m, 1H); LCMS: 91.4%, m/z [M−H]⁻=576.2.

### Synthesis of 250.3a & Synthesis of 250.3b

To a stirred solution of 250.2 (300 mg, 0.51 mmol) and 4-(hydroxymethyl)-5-methyl-1,3-dioxol-2-one (100 mg, 0.77 mmol) in THF (10 mL) were added triphenyl phosphine (160 mg, 0.62 mmol) and DIAD (130 mg, 0.62 mmol) at RT. After stirring for 16 h at RT, the reaction mixture was concentrated under reduced pressure to afford residue. The residue was purified by prep. HPLC [X-SELECT-C18 (150×30) mm, 5 μ; A: 0.1% Formic acid in H₂O, B: Acetonitrile; Gradient: (Time/%B): 0/45, 8/80, 10/80, 10.1/98, 13/98, 13.1/45, 15/45 at 18 mL/min] to afford 250.3a (81 mg, 23%) as a solid and 250.3b (21 mg, 5%) as a solid. 250.3a: ¹H NMR (500 MHz, DMSO-d₆) (Exist as a rotamer): 11.14/11.06 (s, 1H), 8.18/7.76 (d, J=2.0 Hz, 1H), 7.69/7.57 (t, J=2.0 Hz, 1H), 7.45-7.37 (m, 3H), 4.91-4.87 (m, 1H), 4.66/4.59 (d, J=14.5 Hz, 1H), 4.15 (d, J=7.5 Hz, 1H), 3.82-3.79 (m, 1H), 3.66-3.63 (m, 1H), 3.32-3.19 (m, 1H), 3.21 (s, 3H), 2.61-2.56 (m, 1H), 2.38-2.36 (m, 1H), 2.13-2.03 (m, 1H), 2.03/2.02 (s, 3H); LCMS: 98.7%, m/z [M+H]⁺=689.9. 250.3b: ¹H NMR (500 MHz, DMSO-d₆) (Exist as a Rotamer): 8.33/7.91 (d, J=2.0 Hz, 1H), 7.69/7.57 (t, J=2.0 Hz, 1H), 7.53-7.42 (m, 3H), 5.12-5.03 (m, 2H), 4.94-4.91 (m, 1H), 4.54/4.42 (d, J=14.5 Hz, 1H), 4.31/4.23 (d, J=7.5 Hz, 1H), 3.82-3.80 (m, 1H), 3.67-3.65 (m, 1H), 3.41/3.24 (s, 3H), 3.19-3.12 (m, 1H), 2.58-2.50 (m, 1H), 2.41-2.30 (m, 1H), 2.17/2.13 (s, 3H), 2.07-2.04 (m, 1H), 2.02/2.01 (s, 3H); LCMS: 95.5%, m/z [M+H]⁺=801.9.

### Example 251

### Synthesis of rac-(pivaloyloxy)methyl (1′R,2′S,3R,7a′R)-5,7-dichloro-1-((3,5-dichlorophenyl)(methyl)carbamoyl)-6′,6′-difluoro-2-oxo-1′,2′,5′,6′,7′,7a′-hexahydrospiro[indoline-3,3′-pyrrolizine]-2′-carboxylate (251a), (pivaloyloxy)methyl 5,7-dichloro-1-((3,5-dichlorophenyl)(methyl)carbamoyl)-6′,6′-difluoro-2-oxo-1′,2′,5′,6′,7′,7a′-hexahydrospiro[indoline-3,3′-pyrrolizine]-2′-carboxylate (251b) & rac-(pivaloyloxy)methyl (1′R,2′S,3R,7a′R)-5,7-dichloro-1-((3,5-dichlorophenyl)(methyl)carbamoyl)-6′,6′-difluoro-2-oxo-1-((pivaloyloxy)methyl)-1′,2′,5′,6′,7′,7a′-hexahydrospiro[indoline-3,3′-pyrrolizine]-2′-carboxylate (251c)

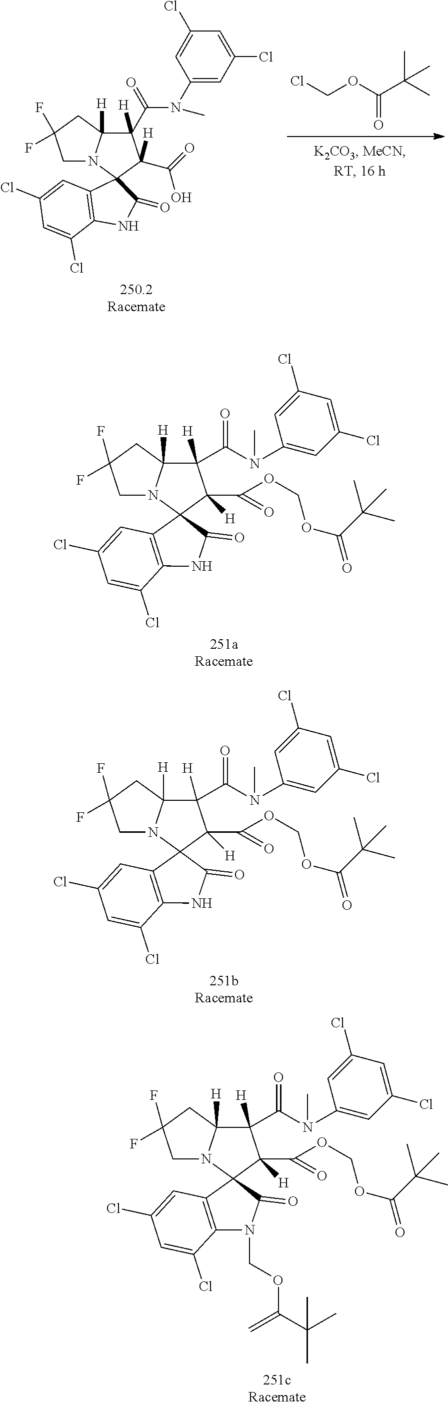

### Synthesis of 251a, 251b & 251c

To a stirred solution of 250.2 (200 mg, 0.35 mmol) and chloromethyl pivalate (80 mg, 0.51 mmol) in CH₃CN (10 mL) was added K₂CO₃ (100 mg, 0.69 mmol) at RT. After stirring for 16 h at RT, the reaction mixture was quenched with water and extracted with EtOAc (10 mL). The combined organic layer was washed with water (5 mL), brine solution (5 mL), dried over Na₂SO₄, filtered and concentrated under reduced pressure. The residue was purified by prep. HPLC [X- BRIDGE-C18 (150×30) mm, 5 μ; A: 0.1% Formic acid in H₂O, B: Acetonitrile; Gradient: (Time/%B): 0/70, 8/90, 10/95, 14/98, 14.1/70, 17/70 at 22 mL/min] to afford 251a (19 mg, 8%) as a solid, 251b (20 mg, 8%) as a solid and 251c (49 mg, 17%) as a solid.

251a: ¹H NMR (500 MHz, DMSO-d₆) (Exist in rotameric form): 11.01/10.91 (br s, 1H), 7.84-7.68 (m, 2H), 7.57-7.30 (m, 3H), 5.54 (s, 2H), 4.34 (d, J=10.5 Hz, 1H), 4.03-3.99 (m, 1H), 3.75-3.73 (m, 1H), 3.48-3.41 (m, 1H), 3.23 (s, 3H), 2.75-2.70 (m, 1H), 2.64-2.50 (m, 1H), 2.37-2.20 (m, 1H), 1.07/1.05 (s, 9H); LCMS: 96.9%, m/z [M+H]⁺=692.0;

251b: ¹H NMR (500 MHz, DMSO-d₆) (Exist in rotameric form): 11.10 (br s, 1H), 8.20 (br s, 1H), 7.71 (t, J=2.0 Hz, 1H), 7.56-7.44 (m, 3H), 5.54-5.47 (m, 2H), 4.14 (d, J=7.5 Hz, 1H), 3.79-3.76 (m, 1H), 3.68-3.66 (m, 1H), 3.40/3.21 (s, 3H), 3.18-3.05 (m, 1H), 2.64-2.51 (m, 1H), 2.42-2.32 (m, 1H), 2.13-2.00 (m, 1H), 1.15-1.03 (m, 9H); LCMS: 95.5%, m/z [M+H]⁺=692.0; (epimerized material-absolute stereochemistry not determined).

251c: ¹H NMR (500 MHz, DMSO-d₆) (Exist in rotameric form): 8.01 (d, J=2.0 Hz, 1H), 7.77 (br s, 1H), 7.68-7.65 (m, 1H), 7.55-7.45 (m, 2H), 5.78-5.71 (m, 2H), 5.56-5.43 (m, 2H), 4.45 (d, J=10.0 Hz, 1H), 3.96-3.92 (m, 1H), 3.76-3.71 (m, 1H), 3.48-3.37 (m, 1H), 3.24 (s, 3H), 2.72-2.67 (m, 1H), 2.55-2.50 (m, 1H), 2.38-2.23 (m, 1H), 1.10-1.06 (m, 18H); LCMS: 93.7%, m/z [M+H]⁺=806.0.

### Example 252

### Synthesis of (5-methyl-2-oxo-1,3-dioxo1-4-yl)methyl (1′R,2′S,3R,7a′R)-5,7-dichloro-1-((3,5-dichlorophenyl)(methyl)carbamoyl)-6′,6′-difluoro-2-oxo-1′,2′,5′,6′,7′,7a′-hexahydrospiro[indoline-3,3′-pyrrolizine]-2′-carboxylate

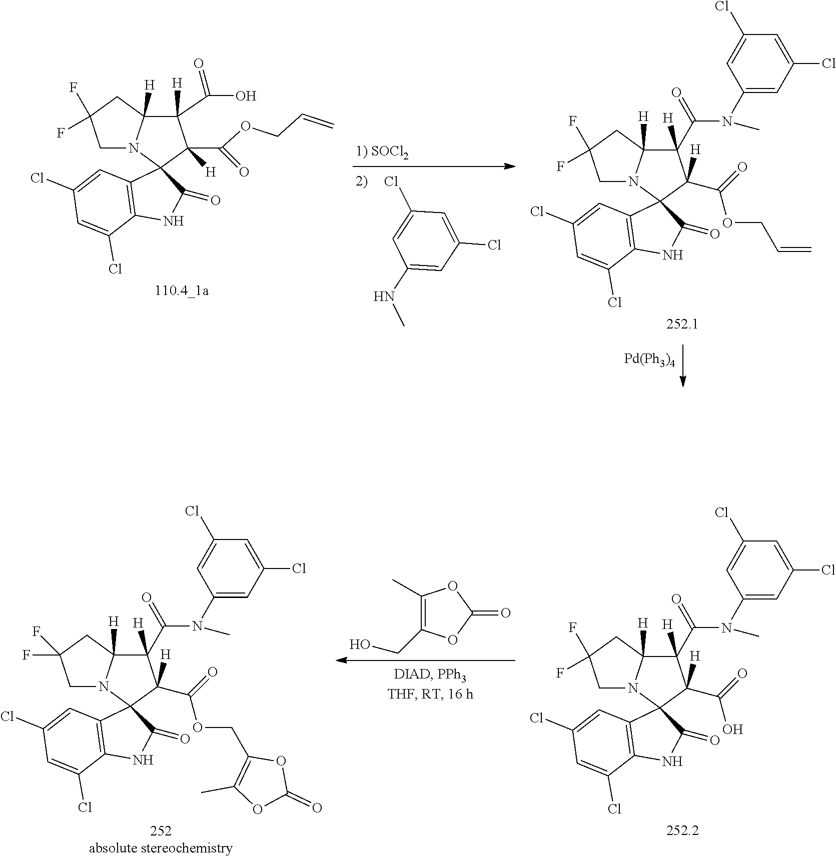

### Synthesis of 252.1

Thionyl chloride (10 mL) was added to 110.4_1a (500 mg, 1.08 mmol) at RT. After stirring for 2 h, the excess thionyl chloride was removed under reduced pressure to afford intermediate acid chloride. To the intermediate acid chloride in CH₂Cl₂ (5 mL) was added a solution of 3,5-dichloro-N-methylaniline (380 mg, 2.16 mmol) in CH₂Cl₂ (5 mL) at RT. After stirring for 16 h at RT, the reaction mixture was quenched with water (10 mL). The organic layer was separated, and the aqueous layer was extracted with CH₂Cl₂ (2×10 mL). The combined organic layer was washed with brine, dried over anhydrous Na₂SO₄, filtered and concentrated under reduced pressure. The residue was purified by flash column chromatography (40 g Silica gel cartridge, 30% EtOAc in pet ether) to afford 252.1 (510 mg, 74%) as a solid.

¹H NMR (500 MHz, DMSO-d₆) (exist in rotameric form): 11.10/11.05 (br s, 1H), 8.20/7.76 (d, J=1.5 Hz, 1H), 7.68/7.57 (t, J=2.0 Hz, 1H), 7.50-7.42 (m, 3H), 5.44-5.38 (m, 1H), 5.10-5.06 (m, 2H), 4.26-4.19 (m, 2H), 4.13 (d, J=7.5 Hz, 1H), 3.84-3.81 (m, 1H), 3.63-3.61 (m, 1H), 3.41/3.24 (s, 3H), 3.32-3.24 (m, 1H), 2.64-2.58 (m, 1H), 2.40-2.36 (m, 1H), 2.19-2.09 (m, 1H); LCMS: 88.4%, m/z [M+H]⁺=620.2.

### Synthesis of 252.2

To a stirred solution of 252.1 (500 mg, 0.80 mmol) in THF (10 mL) were added aniline (46 mg, 0.50 mmol) and Pd(PPh₃)₄ (144 mg, 0.12 mmol) at RT. After stirring for 2 h at RT, the reaction mixture was concentrated under reduced pressure. The residue was purified by prep. HPLC [Column: X-SELECT-C18 (150×30) mm, 5 μ; A: 0.1% Formic acid in H₂O, B: Acetonitrile; Gradient: (Time/%B): 0/45, 8/80, 10/80, 10.1/98, 13/98, 13.1/45, 15/45 at 18 mL/min] to afford 252.2 (165 mg, 35%) as a solid.

¹H NMR (500 MHz, DMSO-d₆) (Exist in rotameric form):12.54 (br s, 1H), 11.07/10.99 (br s, 1H), 8.30/7.88 (d, J=1.5 Hz, 1H), 7.66-7.43 (m, 4H), 4.01 (d, J=7.5 Hz, 1H), 3.84-3.80 (m, 1H), 3.55-3.53 (m, 1H), 3.40/3.24 (s, 3H), 3.32-3.24 (m, 1H), 2.64-2.50 (m, 1H), 2.42-2.32 (m, 1H), 2.14-2.06 (m, 1H); LCMS: 94.7%, m/z [M+H]⁺=578.0; Chiral purity: 99.8%.

### Synthesis of 252

To a stirred solution of 252.2 (300 mg, 0.51 mmol) and 4-(hydroxymethyl)-5-methyl-1,3-dioxol-2-one (101 mg, 0.77 mmol) in THF (5 mL) were added triphenylphosphine (163 mg, 0.62 mmol) and DIAD (125 mg, 0.62 mmol) at RT. After stirring for 16 h at RT, the reaction mixture was concentrated under reduced pressure. The residue was purified by flash chromatography (40 g Silica gel cartridge, 30% EtOAc in pet ether) followed by prep. HPLC [X-SELECT-C₁₈ (150×25) mm, 5 μ; A: 0.1% Formic acid in H₂O, B: Acetonitrile; Gradient: (Time/%B): 0/65, 8/80, 10/90, 10.1/65, 13/65 at 22 mL/min] to afford 252 (120 mg, 33%) as a solid.

¹H NMR (500 MHz, DMSO-d₆) (Exist in rotameric form): 11.14/11.06 (s, 1H), 8.18 (d, J=2.0 Hz, 1H), 7.76-7.69 (m, 1H), 7.57-7.37 (m, 3H), 4.91-4.87 (m, 1H), 4.68-4.65 (m, 1H), 4.23-4.14 (m, 1H), 3.82-3.81 (m, 1H), 3.66-3.63 (m, 1H), 3.32-3.19 (m, 1H), 3.24 (s, 3H), 2.61-2.54 (m, 1H), 2.41-2.36 (m, 1H), 2.09-2.03 (m, 1H), 2.03 (s, 3H); LCMS: 98.1%, m/z [M+H]⁺=689.9; Chiral purity: 97.2%.

### Example 253

### Synthesis of (5-methyl-2-oxo-1,3-dioxo1-4-yl)methyl (1′S,2′R,3S,7a′S)-5,7-dichloro-1-((3,5-dichlorophenyl)(methyl)carbamoyl)-6′,6′-difluoro-2-oxo-1′,2′,5′,6′,7′,7a′-hexahydrospiro[indoline-3,3′-pyrrolizine]-2′-carboxylate

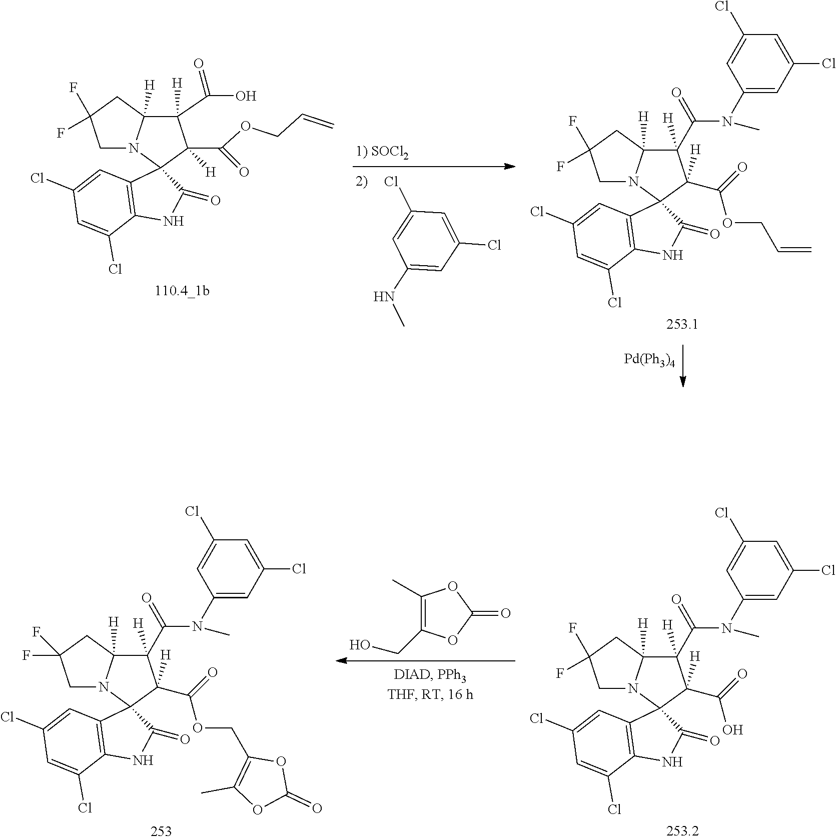

### Synthesis of 253.1

Thionyl chloride (8 mL) was added to 110.4_1b (400 mg, 0.86 mmol) at RT. After stirring for 2 h, the excess thionyl chloride was removed under reduced pressure to afford intermediate acid chloride. To this acid chloride in CH₂Cl₂ (5 mL) was added a solution of 3,5-dichloro-N-methylaniline (308 mg, 1.75 mmol) in CH₂Cl₂ (5 mL) at RT. After stirring for 16 h at RT, the reaction was quenched with water (10 mL). The organic layer was separated, and the aqueous layer was extracted with CH₂Cl₂ (10 mL×2). The combined organic layer was washed with brine, dried over anhydrous Na₂SO₄, filtered and concentrated under reduced pressure. The residue was purified by flash column chromatography (40 g Silica gel cartridge, 30% ethyl acetate in pet ether) to afford 253.1 (410 mg, 74%) as a solid.

¹H NMR (500 MHz, DMSO-d₆) (exist in rotameric form): 11.13/11.05 (br s, 1H), 8.20/7.76 (d, J=2.0 Hz, 1H), 7.68/7.57 (t, J=2.0 Hz, 1H), 7.50-7.42 (m, 3H), 5.42-5.38 (m, 1H), 5.10-5.06 (m, 2H), 4.26-4.19 (m, 2H), 4.13 (d, J=7.5 Hz, 1H), 3.84-3.81 (m, 1H), 3.63-3.61 (m, 1H), 3.41/3.24 (s, 3H), 3.32-3.24 (m, 1H), 2.64-2.58 (m, 1H), 2.42-2.34 (m, 1H), 2.20-2.09 (m, 1H); LCMS: 84.1%, m/z [M+H]⁺=620.2.

### Synthesis of 253.2

To a stirred solution of 253.1 (400 mg, 0.64 mmol) in THF (10 mL) were added aniline (60 mg, 0.64 mmol) and Pd(PPh₃)₄ (138 mg, 0.12 mmol) at RT. After stirring for 2 h at RT. After completion of the reaction, the reaction mixture was concentrated under reduced pressure. The residue was purified by prep. HPLC [Column: X-SELECT-C18 (150×30) mm, 5 μ; A: 0.1% HCOOH in H₂O, B: Acetonitrile; Gradient: (Time/%B): 0/45, 8/80, 10/80, 10.1/98, 13/98, 13.1/45, 15/45 at 18 mL/min] to afford 253 (140 mg, 37%) as a solid.

¹H NMR (500 MHz, DMSO-d₆) (Exist in rotameric form): 12.55 (br s, 1H), 11.07/11.00 (s, 1H), 8.30/7.88 (d, J=2.0 Hz, 1H), 7.66/7.56 (t, J=2.0 Hz, 1H), 7.49-7.43 (m, 2H), 4.02 (d, J=8.0 Hz, 1H), 3.84-3.80 (m, 1H), 3.56-3.53 (m, 1H), 3.40/3.24 (s, 3H), 3.32-3.24 (m, 1H), 2.64-2.54 (m, 1H), 2.41-2.31 (m, 1H), 2.17-2.02 (m, 1H); LCMS: 90.8%, [M+H]⁺=578.0; Chiral purity: 99.8%.

### Synthesis of 253

To a stirred solution of 253.2 (300 mg, 0.51 mmol) and 4-(hydroxymethyl)-5-methyl-1,3-dioxol-2-one (101 mg, 0.77 mmol) in THF (5 mL) was added triphenyl phosphine (163 mg, 0.62 mmol) and DIAD (125 mg, 0.62 mmol) at RT. After stirring for 16 h at RT, the reaction mixture was concentrated under reduced pressure. The residue was purified by flash chromatography (40 g Silica gel cartridge, 30% EtOAc in pet ether) followed by prep. HPLC [KROMOSIL-C18 (150×25) mm, 10 μ; A: 0.1% Formic acid in H₂O, B: Acetonitrile; Gradient: (Time/%B): 0/50, 8/70, 12/98, 14/98, 14.1/50, 16/50 at 22 mL/min] to afford 253 (134 mg, 37%) as a solid.

¹H NMR (500 MHz, DMSO-d₆) (Exist in rotameric form): 11.10/11.06 (s, 1H), 8.18 (d, J=2.0 Hz, 1H), 7.76-7.69 (m, 1H), 7.57-7.37 (m, 3H), 4.91-4.87 (m, 1H), 4.68-4.65 (m, 1H), 4.23-4.14 (m, 1H), 3.82-3.81 (m, 1H), 3.66-3.63 (m, 1H), 3.31-3.21 (m, 1H), 3.24 (s, 3H), 2.60-2.54 (m, 1H), 2.40-2.36 (m, 1H), 2.11-2.02 (m, 1H), 2.02 (s, 3H); LCMS: 99.0%, m/z [M+H]⁺=689.9; Chiral purity: 99.5%.

### Example 254

### Synthesis of (pivaloyloxy)methyl (1′R,2′S,3R,7a′R)-5,7-dichloro-1-((3,5-dichlorophenyl)(methyl)carbamoyl)-6′,6′-difluoro-2-oxo-1′,2′,5′,6′,7′,7a′-hexahydrospiro[indoline-3,3′-pyrrolizine]-2′-carboxylate (254a) and (pivaloyloxy)methyl (1′R,2′R,3R,7a′R)-5,7-dichloro-1′-((3,5-dichlorophenyl)(methyl)carbamoyl)-6′,6′-difluoro-2-oxo-1′,2′,5′,6′,7′,7a′-hexahydrospiro[indoline-3,3′-pyrrolizinel-2′-carboxylate (254b):

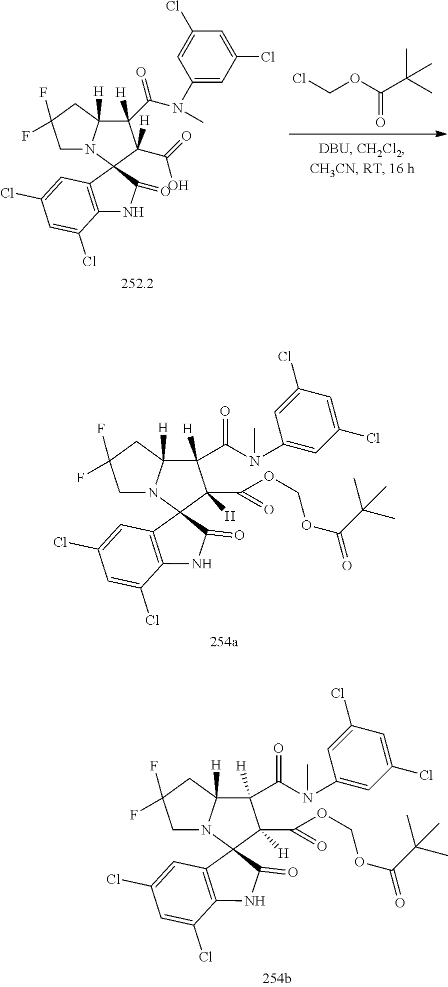

To a stirred solution of 252.2 (350 mg, 0.60 mmol) and chloromethyl pivalate (273 mg, 1.81 mmol) in CH₂Cl₂:CH₃CN (4:1, 5 mL) was added DBU (275 mg, 1.81 mmol) at RT. After stirring for 16 h at RT, the reaction mixture was washed with water (5 mL), brine (5 mL), dried over Na₂SO₄, filtered, and concentrated under reduced pressure. The residue was purified by prep. HPLC [X BRIDGE-C18 (150×25) mm, 5 μ; A: 0.1% Formic acid in H₂O, B: Acetonitrile; Gradient: (Time/%B): 0/65, 8/85, 10/90, 14/98, 17/98, 17.1/65, 20/65 at 24 mL/min] to afford 254a (95 mg, 22%) as a solid and 254b (58 mg, 14%) (epimerized material) as a solid.

254a: ¹H NMR (500 MHz, DMSO-d₆) (Exist in rotameric form): 11.15/11.06 (br s, 1H), 8.20 (d, J=1.5 Hz, 1H), 7.84-7.70 (m, 1H), 7.55-7.44 (m, 3H), 5.54-5.47 (m, 2H), 4.15-4.13 (m, 1H), 3.80-3.76 (m, 1H), 3.68-3.65 (m, 1H), 3.21 (s, 3H), 3.20-3.10 (m, 1H), 2.60-2.54 (m, 1H), 2.38-2.33 (m, 1H), 2.07-2.04 (m, 1H), 1.07/1.03 (m, 9H); LCMS: 99.4%, m/z [M+H]⁺=691.9; Chiral purity: 94.6%. Stereochemistry was confirmed by 2D NMR studies.

254b: ¹H NMR (500 MHz, DMSO-d₆) (Exist in rotameric form): 11.08/10.92 (br s, 1H), 7.84 (d, J=1.5 Hz, 1H), 7.77 (s, 1H), 7.54-7.44 (m, 3H), 5.60-5.50 (m, 2H), 4.34 (d, J=10.5 Hz, 1H), 4.11-3.97 (m, 1H), 3.77-3.71 (m, 1H), 3.50-3.41 (m, 1H), 3.23 (s, 3H), 2.75-2.70 (m, 1H), 2.55-2.45 (m, 1H), 2.31-2.20 (m, 1H), 1.07 (s, 9H); LCMS: 97.8%, m/z [M+H]⁺=691.9; Chiral purity: 95.7%. Stereochemistry was confirmed by 2D NMR studies.

### Example 255

### Synthesis of (pivaloyloxy)methyl (1′S,2′R,3S,7a′S)-5,7-dichloro-1-((3,5-dichlorophenyl)(methyl)carbamoyl)-6′,6′-difluoro-2-oxo-1′,2′,5′,6′,7′,7a′-hexahydrospiro[indoline-3,3′-pyrrolizine]-2′-carboxylate (255a) and (pivaloyloxy)methyl (1′S,2′S,3S,7a′S)-5,7-dichloro-1-((3,5-dichlorophenyl)(methyl)carbamoyl)-6′,6′-difluoro-2-oxo-1′,2′,5′,6′,7′,7a′-hexahydrospiro[indoline-3,3′-pyrrolizine]-2′-carboxylate (255b):

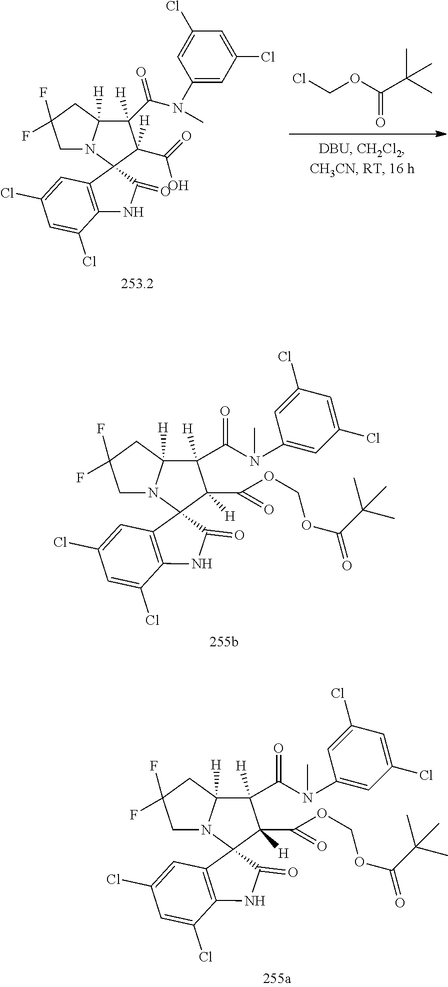

To a stirred solution of 253.2 (350 mg, 0.60 mmol) and chloromethyl pivalate (273 mg, 1.81 mmol) in CH₂Cl₂:CH₃CN (4:1, 5 mL) was added DBU (275 mg, 1.81 mmol) at RT. After stirring for 16 h at RT, the reaction mixture was washed with water (5 mL), brine solution (5 mL), dried over Na₂SO₄, filtered and concentrated under reduced pressure. The residue was purified by prep. HPLC [KROMOSIL-C18 (150×25) mm, 10 μ; A: 0.1% Formic acid in H₂O, B: Acetonitrile; Gradient: (Time/%B): 0/70, 8/90, 10/95, 12/98, 14/98, 14.1/70, 16/70 at 18 mL/min] to afford 255a (65 mg, 15%) as a solid and 255b (108 mg, 25%) as a solid. 255a: ¹H NMR (500 MHz, DMSO-d₆) (Exist in rotameric form): 11.07/10.91 (s, 1H), 7.84 (s, 1H), 7.77 (s, 1H), 7.54-7.44 (m, 3H), 5.56-5.54 (m, 2H), 4.43-4.33 (m, 1H), 4.03-3.99 (m, 1H), 3.77-3.71 (m, 1H), 3.50-3.40 (m, 1H), 3.23 (s, 3H), 2.75-2.64 (m, 1H), 2.54-2.50 (m, 1H), 2.47-2.25 (m, 1H), 1.07 (s, 9H); LCMS: 99.2%, m/z [M+H]⁺=692.0; Chiral purity: 99.6%.

255b: ¹H NMR (500 MHz, DMSO-d₆) (Exist in rotameric form): 11.06/10.90 (br s, 1H), 8.20 (d, J=1.5 Hz, 1H), 7.84-7.70 (m, 1H), 7.55-7.45 (m, 3H), 5.54-5.47 (m, 2H), 4.18-4.13 (m, 1H), 3.80-3.76 (m, 1H), 3.68-3.65 (m, 1H), 3.21 (s, 3H), 3.21-3.10 (m, 1H), 2.65-2.50 (m, 1H), 2.40-2.30 (m, 1H), 2.11-1.98 (m, 1H), 1.15-1.03 (m, 9H); LCMS: 99.6%, m/z [M+H]⁺=691.9; Chiral purity: 97.1%.

### Example 256

### Synthesis of acetoxymethyl (1′R,2′R,3R,7a′R)-5,7-dichloro-1′-((3,5-dichlorophenyl)(methyl)carbamoyl)-6′,6′-difluoro-2-oxo-1′,2′,5′,6′,7′,7a′-hexahydrospiro[indoline-3,3′-pyrrolizine]-2′-carboxylate (256a) and acetoxymethyl (1′R,2′S,3R,7a′R)-5,7-dichloro-1′-((3,5-dichlorophenyl)(methyl)carbamoyl)-6′,6′-difluoro-2-oxo-1′,2′,5′,6′,7′,7a′-hexahydrospiro[indoline-3,3′-pyrrolizine]-2′-carboxylate (256b):

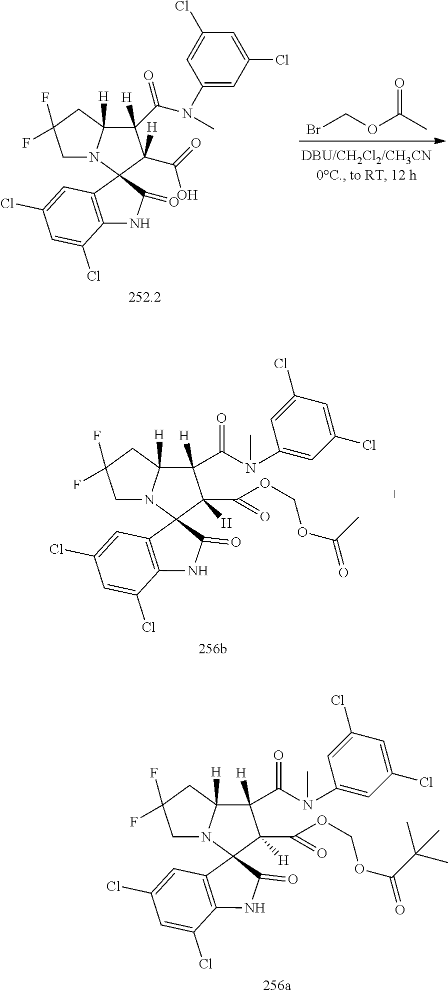

To a stirred solution of 252.2 (300 mg, 0.52 mmol) in DCM (6 mL) and CH₃CN (1.5 mL) were added DBU (87 mg, 0.52 mmol) and bromomethyl acetate (87 mg, 0.52 mmol) at 0° C. After stirring at RT for 12 h, the reaction mixture was diluted with water (30 mL) and extracted with DCM (2×50 mL). The combined organic layer was washed with brine (50 mL), dried over anhydrous Na₂SO₄, filtered and concentrated under reduced pressure. The residue was purified by prep. HPLC (Column: X-BRIDGE-C18 (150×30) mm, 5 μ; A: 0.1% Formic Acid in H2O, B: Acetonitrile; Gradient: 0/60,8/80,10/85,13/98,15/98,15.1/60,18/60 at 23 mL/min) to afford 256a (Peak-1, 30 mg, 9%) as an off white solid and 256b (Peak-2, 60 mg, 18%) as a solid.

256a: ¹H NMR (500 MHz, DMSO-d₆) (Exist in rotameric form): 11.10/10.93 (s, 1 H), 7.81 (d, J=1.5 Hz, 1H), 7.77 (s, 1H), 7.56-7.44 (m, 3H), 5.54 (d, J=6.0 Hz, 1H), 5.45 (d, J=6.0 Hz, 1H), 4.33 (d, J=10.5 Hz, 1H), 4.01-3.97 (m, 1H), 3.78-3.73 (m, 1H), 3.51-3.44 (m, 1H), 3.23 (s, 3H), 2.77-2.72 (m, 1H), 2.51-2.49 (m, 1H), 2.37-2.36 (m, 1H), 2.01 (s, 3H); LCMS: 96.7%, m/z [M+H]⁺=650.0; Chiral purity: 99.9%.

256b: ¹H NMR (500 MHz, DMSO-d₆) (Exist in rotameric form): 11.10/11.05 (s, 1H), 8.18/7.75 (d, J=2.0 Hz, 1H), 7.70 (t, J=2.0 Hz, 1H), 7.58-7.45 (m, 3H), 5.52/5.48 (d, J=6.0 Hz, 1H), 5.40-5.38 (m, 1H), 4.14 (d, J=7.5 Hz, 1H), 3.80-3.77 (m, 1H), 3.68-3.65 (m, 1H), 3.40/3.23 (s, 3H), 3.21-3.17 (m, 1H), 2.64-2.63 (m, 1H), 2.37-2.35 (m, 1H), 2.11-2.08 (m, 1H), 1.97/1.89 (s, 3H); LCMS: 98.0%, m/z [M+H]⁺=650.1; Chiral purity: 99.5%.

### Example 257

### Synthesis of acetoxymethyl (1′R,2′S,3R,7a′R)-5,7-dichloro-1-((3,5-dichlorophenyl)(methyl)carbamoyl)-6′,6′-difluoro-2-oxo-1′,2′,5′,6′,7′,7a′-hexahydrospiro[indoline-3,3′-pyrrolizine]-2′-carboxylate:

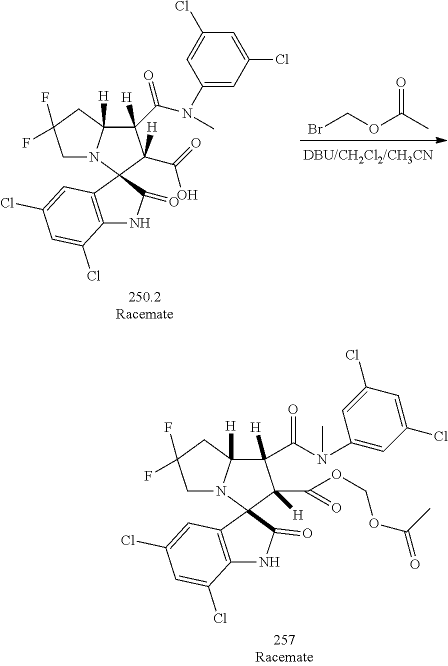

257 was synthesized from 250.2 following the procedure described for the synthesis of 256a and 256b.

¹H NMR (500 MHz, DMSO-d₆) (Exist in rotameric form): 11.15/11.05 (s, 1H), 8.18/7.75 (d, J=2.0 Hz, 1H), 7.69/7.57 (t, J=2.0 Hz, 1H), 7.51-7.45 (m, 3H), 5.52/5.48 (d, J=6.0 Hz, 1H), 5.40-5.38 (m, 1H), 4.14 (d, J=7.5 Hz, 1H), 3.82-3.77 (m, 1H), 3.66 (t, J=7.0 Hz, 1H), 3.40/3.23 (s, 3H), 3.21-3.17 (m, 1H), 2.61-2.51 (m, 1H), 2.42-2.30 (m, 1H), 2.15-2.00 (m, 1H), 1.97/1.89 (s, 3H); LCMS: 97.5%, m/z [M+H]⁺=650.

### Example 258

### Synthesis of 2-(dimethylamino)-2-oxoethyl (1′R,2′S,3R,7a′R)-5,7-dichloro-1′-((3,5-dichlorophenyl)(methyl)carbamoyl)-6′,6′-difluoro-2-oxo-1′,2′,5′,6′,7′,7a′-hexahydrospiro[indoline-3,3′-pyrrolizine]-2′-carboxylate:

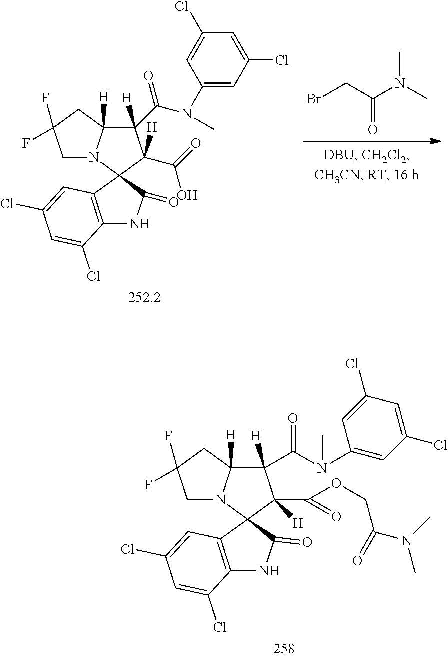

To a stirred solution of 252.2 (400 mg, 0.69 mmol) and 2-bromo-N,N-dimethylacetamide (230 mg, 1.38 mmol) in CH₂Cl₂:CH₃CN (4:1, 8 mL) was added DBU (210 mg, 1.38 mmol) at RT. After stirring for 16 h at RT, the reaction mixture was diluted with CH₂Cl₂ (10 mL). The organic layer was washed with water (10 mL), brine (10 mL), dried over Na₂SO₄ and filtered. The organic layer was concentrated under reduced pressure. The residue was purified by prep. HPLC [Column: X-BRIDGE-C18 (150×30) mm, 5 μ; A: 0.1% Formic acid in H₂O, B: Acetonitrile; Gradient: (Time/%B): 0/60, 8/80, 11/90, 11.1/98, 13/98, 13.1/60, 16/60 at 18 mL/min] to afford 258 (198 mg, 43%) as a solid.

¹H NMR (500 MHz, DMSO-d₆) (Exist in rotameric form): 11.13/11.05 (s, 1H), 8.17/7.75 (d, J=2.0 Hz, 1H), 7.68-7.67 (m, 1H), 7.56-7.45 (m, 3H), 4.69-4.44 (m, 2H), 4.25-4.07 (m, 1H), 3.84-3.81 (m, 1H), 3.74-3.71 (m, 1H), 3.22 (s, 3H), 3.19-3.00 (m, 1H), 2.83/2.82 (s, 3H), 2.77 (s, 3H), 2.63-2.57 (m, 1H), 2.35-2.34 (m, 1H), 2.13-2.10 (m, 1H); LCMS: 99.2%, m/z [M+H]⁺=663.2; Chiral purity: 97.3%.

### Example 259

### Synthesis of S-(2-acetamidoethyl) (1′R,2′S,3R,7a′R)-5,7-dichloro-1′-((3,5-dichlorophenyl)(methyl)carbamoyl)-6′,6′-difluoro-2-oxo-1′,2′,5′,6′,7′,7a′-hexahydrospiro[Iindoline-3,3′-pyrrolizine]-2′-carbothioate:

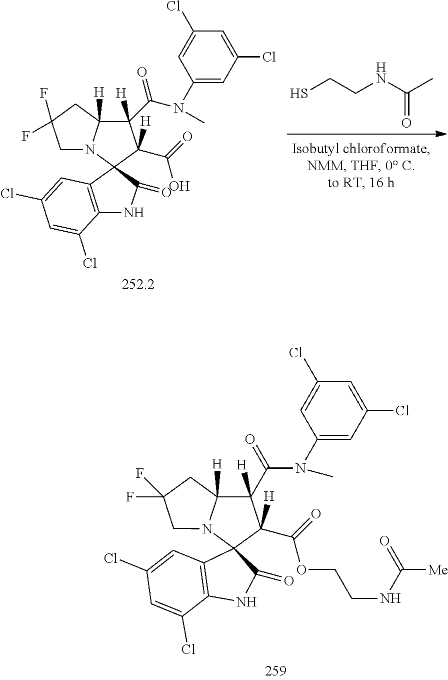

To a stirred solution of 252.2 (400 mg, 0.69 mmol) in THF (8 mL) was added N-methylmorpholine (140 mg, 1.38 mmol) and isobutyl chloroformate (141 mg, 1.38 mmol) at 0° C. After 30 minutes, N-(2-mercaptoethyl)acetamide (246 mg, 2.07 mmol) was added at 0° C. After stirring for 16 h at RT, the reaction mixture was quenched with water and extracted with EtOAc (2×10 mL). The combined organic layer was washed with water (20 mL), brine (20 mL), dried over Na₂SO₄, filtered and concentrated under reduced pressure. The residue was purified by prep. HPLC [Column: KROMASIL C18 (150×25) mm, 10 μ; A: 10 mM Ammonium bicarbonate in H₂O, B: Acetonitrile; Gradient: (Time/%B): 0/50, 8/80, 10/80, 10.1/98, 11/98, 11.1/50, 14/50 at 20 mL/min] to afford 259 (38 mg, 8%) as a solid.

¹H NMR (500 MHz, DMSO-d₆) (Exist in rotameric form): 11.00 (br s, 1H), 7.93-7.76 (m, 3H), 7.57-7.46 (m, 3H), 4.68-4.35 (m, 1H), 4.07-4.03 (m, 1H), 3.80-3.79 (m, 1H), 3.54-3.49 (m, 1H), 3.27 (s, 3H), 3.00-2.98 (m, 2H), 2.78-2.73 (m, 3H), 2.57-2.50 (m, 1H), 2.36-2.23 (m, 1H). 1.75 (s, 3H); LCMS: 98.6%, m/z [M+H]⁺=679.2.

### Example 260

### Synthesis of (1′R,2′S,3R,7a′R)-5,7-dichloro-N1′-(3,5-dichlorophenyl)-6′,6′-difluoro-N2′-hydroxy-N1′,N2′-dimethyl-2-oxo-1′,2′,5′,6′,7′,7a′-hexahydrospiro[indoline-3,3′-pyrrolizine]-1′,2′-dicarboxamide (260a) and (1′R,2′R,3R,7a′R)-5,7-dichloro-N1′-(3,5-dichlorophenyl)-6′,6′-difluoro-N2′-hydroxy-N1′,N2′-dimethyl-2-oxo-1′,2′,5′,6′,7′,7a′-hexahydrospiro[indoline-3,3′-pyrrolizine]-1′,2′-dicarboxamide (260b):

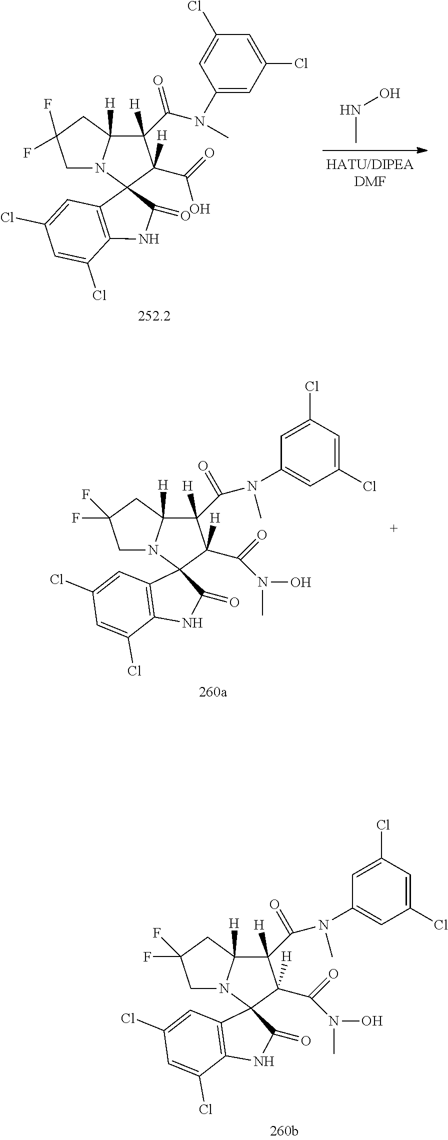

To a stirred solution of 252.2 (900 mg, 1.55 mmol) in DMF (20 mL) were added HATU (709 mg, 1.86 mmol) and DIPEA (0.5 mL, 3.10 mmol,) at RT. After 15 minutes, N-methylhydroxylamine (193 mg, 2.33 mmol) was added. After stirring for 16 h at RT, the reaction mixture was diluted with ice-water (20 mL) and extracted with EtOAc (2×20 mL). The combined organic layer was washed with cold water (20 mL), brine (20 mL), dried over anhydrous Na₂SO₄, filtered and concentrated under reduced pressure. The residue was purified by prep. HPLC [Column: KROMASIL C18 (150×25) mm, 10 μ; A: 0.1% Formic acid in H₂O, B: Acetonitrile; Gradient: (Time/%B): 0/50, 8/65, 11/65, 11.1/98, 12/98, 12.1/50, 14/50 at 23 mL/min] to afford 260a (83 mg) as off-white solid and 260b (55 mg, 6%) as off-white solid.

260a: ¹H NMR (500 MHz, DMSO-d₆) (Exist in rotameric form): 10.88/10.78 (br s, 1 H), 10.07/9.95 (br s, 1H), 7.72 (s, 1H), 7.54-7.30 (m, 4H), 4.63-4.41 (m, 1H), 4.12 (m, 1H), 3.51-3.39 (m, 2H), 3.37/3.22 (s, 3H), 2.99/2.97 (s, 3H), 2.74-2.69 (m, 1H), 2.60-2.50 (m, 1H), 2.22-2.10 (m, 1H); LCMS: 98.3%, m/z [M+H]⁺=607.2; Chiral Purity: 98.9%.

260b: ¹H NMR (500 MHz, DMSO-d₆) (Exist in rotameric form): 10.69 (s, 1H), 9.81 (s, 1H), 7.93 (s, 1H), 7.63 (s, 1H), 7.51 (s, 2H), 7.45 (d, J=2.0 Hz, 1H), 4.07 (d, J=7.5 Hz, 1H), 3.83-3.79 (m, 1H), 3.50-3.44 (m, 1H), 3.36 (t, J=6.5 Hz, 1H), 3.24 (s, 3H), 2.72 (s, 3H), 2.50-2.33 (m, 2H), 2.21-2.12 (m, 1H); LCMS: 95.4%, m/z [M+H]⁺=607.1; Chiral Purity: 87.9%.

### Example 261

### Synthesis of (1′R,2′S,3R,7a′R)-5,7-dichloro-N1′-(3,5-dichlorophenyl)-6′,6′-difluoro-N1′-methyl-2-oxo-N2′-(phenylsulfonyl)-1′,2′,5′,6′,7′,7a′-hexahydrospiro[indoline-3,3′-pyrrolizine]-1,2′-dicarboxamide:

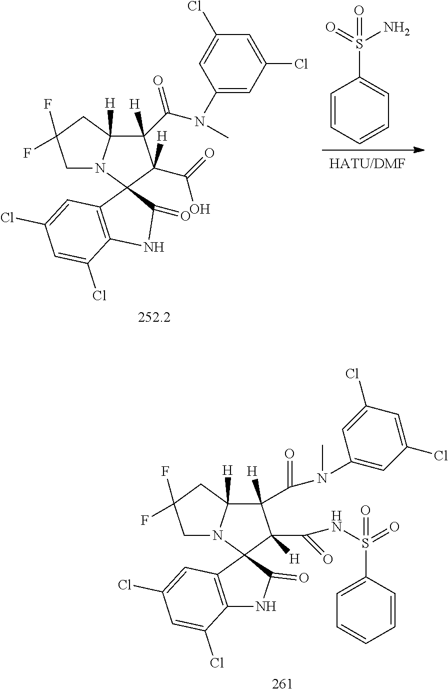

To a stirred solution of 252.2 (400 mg, 0.69 mmol) in DMF (6 mL) were added HATU (395 mg, 1.04 mmol) and DIPEA (0.23 mL, 1.39 mmol) at RT. After 15 minutes, benzenesulfonamide (163 mg, 1.04 mmol) was added. After stirring at RT for 48 h, the reaction mixture was diluted with ice-cold water (80 mL) and extracted with EtOAc (2×50 mL). The combined organic layer was washed with brine (50 mL), dried over anhydrous Na₂SO₄, filtered and concentrated under reduced pressure. The residue was purified by prep. HPLC [Column: X BRIDGE C18 (150×30) mm, 5 μ; A: 0.1% Formic acid in H₂O, B: Acetonitrile; Gradient: 0/60, 8/75, 10/75, 10.1/98, 12/98, 12.1/60, 15/60 at 23 mL/min] to afford 261 (102 mg, 21%) as a solid.

¹H NMR (300 MHz, DMSO-d₆) (Exist in rotameric form): 12.00 (br s, 1H), 10.85/10.65 (br s, 1H), 7.73-7.40 (m, 10H), 4.65-4.52 (m, 1H), 4.28-4.10 (m, 1H), 4.01-3.92 (m, 1H), 3.70-3.60 (m, 1H), 3.20 (s, 3H), 3.00-2.80 (m, 1H), 2.51-2.40 (m, 1H), 2.15-2.00 (m, 1H); LCMS: 94.2%, m/z [M+H]⁺=716.9; Chiral purity: 94.5%.

### Example 265

### Synthesis of (1′S,2′R,3S,7a′S)-5,7-dichloro-1-((3,5-dichloro-2-fluorophenyl)(methyl)carbamoyl)-6′,6′-difluoro-2-oxo-1′,2′,5′,6′,7′,7a′-hexahydrospiro[indoline-3,3′-pyrrolizine]-2′-carboxylic acid (265a) & (1′R,2′S,3R,7a′R)-5,7-dichloro-1-((3,5-dichloro-2-fluorophenyl)(methyl)carbamoyl)-6′,6′-difluoro-2-oxo-1′,2′,5′,6′,7′,7a′-hexahydrospiro[indoline-3,3′-pyrrolizine]-2′-carboxylic acid (265b):

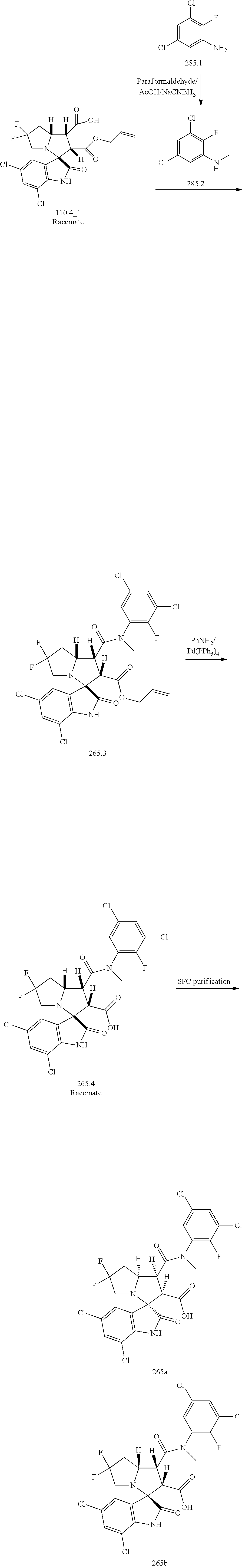

### Synthesis of 265.2

To a stirred solution of 265.1 (500 mg, 2.77 mmol) in MeOH (10 mL) were added paraformaldehyde (0.083 g, 2.77 mmol) and AcOH (0.1 mL, cat.) at RT. After stirring for 3 h at RT, NaCNBH₃ (523 mg, 8.33 mmol) was added. After stirring for 16 h at 60° C., the reaction mixture was evaporated under reduced pressure, diluted with H₂O (25 mL) and extracted with EtOAc (2×15 mL). The combined organic layer was dried over anhydrous Na₂SO₄, filtered and concentrated under reduced pressure. The residue was purified by (Silica gel 100-200 mesh, 2% EtOAc/hexane) to afford 265.2 (200 mg, 37%) as a yellow liquid.

¹H NMR (400 MHz, CDCl₃): 6.69-6.63 (m, 1H), 6.55-6.49 (m, 1H), 4.12 (br s, 1H), 2.87 (d, J=5.2 Hz, 3H); LCMS: 94.7%, m/z [M+H]⁺=194.0.

### Synthesis of 265.3

Thionyl chloride (5 mL) was added to compound 110.4_1 (350 mg, 0.76 mmol) at RT. After stirring for 2 h, the reaction mixture was concentrated under reduced pressure. To the intermediate acid chloride in CH₂Cl₂ (3 mL) was added a solution of 265.2 (221 mg, 1.13 mmol) in CH₂Cl₂ (2 mL). After stirring for 16 h at RT, the reaction mixture was quenched with water (25 mL) and extracted with CH₂Cl₂ (2×15 mL). The combined organic layer was washed with brine, dried over anhydrous Na₂SO₄, filtered and concentrated under reduced pressure. The residue was purified by (Silica gel 100-200 mesh, 10% EtOAc/hexane) to afford 265.3 (200 mg, 41%) as a solid. LCMS: 57.1%, m/z [M−H]⁻=636.2.

### Synthesis of 265a & 265b

To a stirred solution of 265.3 (200 mg, 0.31 mmol) in THF (5 mL) were added aniline (29.0 mg, 0.31 mmol) and Pd(PPh₃)₄ (72.0 mg, 0.062 mmol) at RT. After stirring for 2 h at RT, the reaction mixture was concentrated under reduced pressure. The residue was purified by chiral SFC [Chiralpak IE (30×250) mm, 5 μ; 80% CO₂: 20% of 0.5% isopropylamine in isopropanol at RT (Isocratic 70 g/min, with detection at 214 nm) to afford 265a (7 mg) and 265b (7 mg) as a solid.

265a: ¹H NMR (300 MHz, DMSO-d₆) (Exist in rotameric form): 12.83-12.14 (br s, 1H), 10.99 (br s, 1H), 8.26/8.22 (s, 1H), 7.88-7.82 (m, 2H), 7.70-7.43 (m, 1H), 4.15-3.88 (m, 3H), 3.31-3.26 (m, 1H), 3.20 (s, 3H), 2.73-2.43 (m, 2H), 2.10-1.95 (m, 1H); LCMS: 98.1%, m/z [M+H]⁺=595.9, Chiral purity: 96.9%. Absolute stereochemistry was not determined.

265b: ¹H NMR (300 MHz, DMSO-d₆) (Exist in rotameric form): 11.19-10.24 (br s, 1H), 8.28/8.22 (br s, 1H), 7.83-7.38 (m, 3H), 4.13-3.83 (m, 4H), 3.32-3.25 (m, 1H), 3.18 (s, 3H), 2.60-2.44 (m, 1H), 2.30-2.25 (m, 1H); LCMS: 95.7%, m/z [M+H]⁺=595.9; Chiral purity: 90.9%. Absolute stereochemistry was not determined.

### Example 266

### Synthesis of (1′R,2′S,7a′R)-5,7-dichloro-1-((3,5-dichlorophenyl)(methyl)carbamoyl)-2-oxo-1,2′,5′,6′,7′,7a′-hexahydrospiro[indoline-3,3′-pyrrolizine]-2′-carboxylic acid

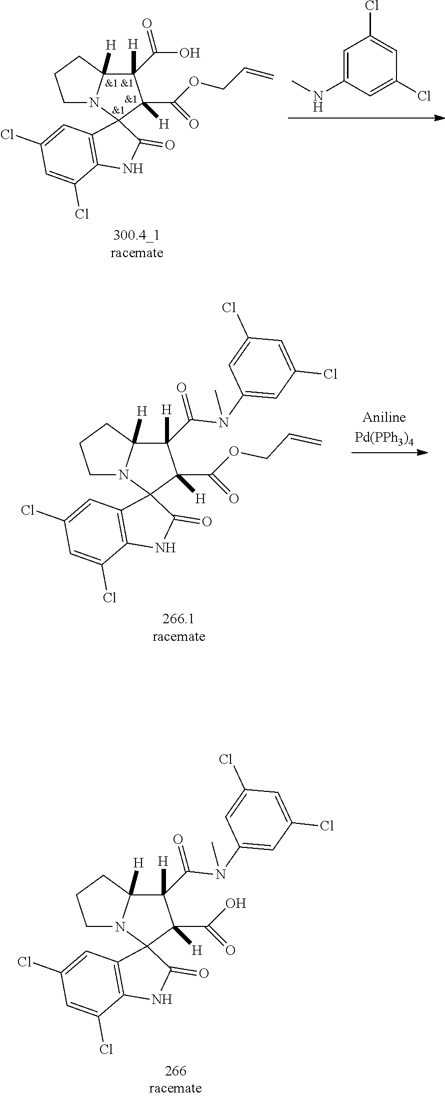

### Synthesis of 266.1

Thionyl chloride (10 mL) was added to 82.4_1 (500 mg, 1.17 mmol). After stirring at RT for 2 h, the reaction mixture was concentrated under reduced pressure to afford acid chloride intermediate. To the acid chloride intermediate was added a solution of 3,5-dichloro-N-methylaniline (395 mg, 2.26 mmol) in CH₂Cl₂ (10 mL). After stirring at RT for 16 h, the reaction mixture was concentrated under reduced pressure. The residue was purified by flash chromatography (Silica gel, 15% EtOAc/pet ether) to afford 266.1 (200 mg, 29%) as a pale brown solid. LCMS: 96.1%, m/z [M−H]⁻=581.8.

### Synthesis of 266

To a stirred solution of 266.1 (100 mg, 0.17 mmol) in THF (5 mL) were added aniline (16.1 mg, 0.17 mmol) and Pd(PPh₃)₄ (39.6 mg, 0.03 mmol) at RT. After stirring for 2 h at RT, the reaction mixture was concentrated under reduced pressure. The residue was purified by prep. HPLC [Kromasil C8 (150×25), 10 μ; A: 0.1% Formic acid in H₂O, B: Acetonitrile; Gradient:(T%B): −0/60, 8/90, 9/80, 9.1/80, 10/98, 10.1/60, 14/60 at 25 mL/min] to afford 266 (60 mg, 9% yield) as a solid.

¹H NMR (500 MHz, DMSO-d₆) (Exist in rotameric form): 12.47 (brs, 1H), 10.98 (br s, 1H), 8.41/7.91 (d, J=1.5 Hz, 1H), 7.68-7.54 (m, 1H), 7.47-7.40 (m, 3H), 4.09 (d, J=8.0 Hz, 1H), 3.80-3.77 (m, 1H), 3.59-3.50 (m, 1H), 3.41/3.23(s, 3H), 2.88-2.79 (m, 1H), 2.30-2.19 (m, 1H), 1.98-1.88 (m, 1H), 1.75-1.70 (m, 2H), 1.60-1.50 (m, 1H); LCMS: 91.4%, m/z [M+H]⁺=542.1.

### Example 267

### Synthesis of 1′-((3-(tert-butyl)phenethyl)carbamoyl)-5,7-dichloro-7′,7′-difluoro-2-oxo-1′,2′,5′,6′,7′,7a′-hexahydrospiro[indoline-3,3′-pyrrolizine]-2′-carboxylic acid:

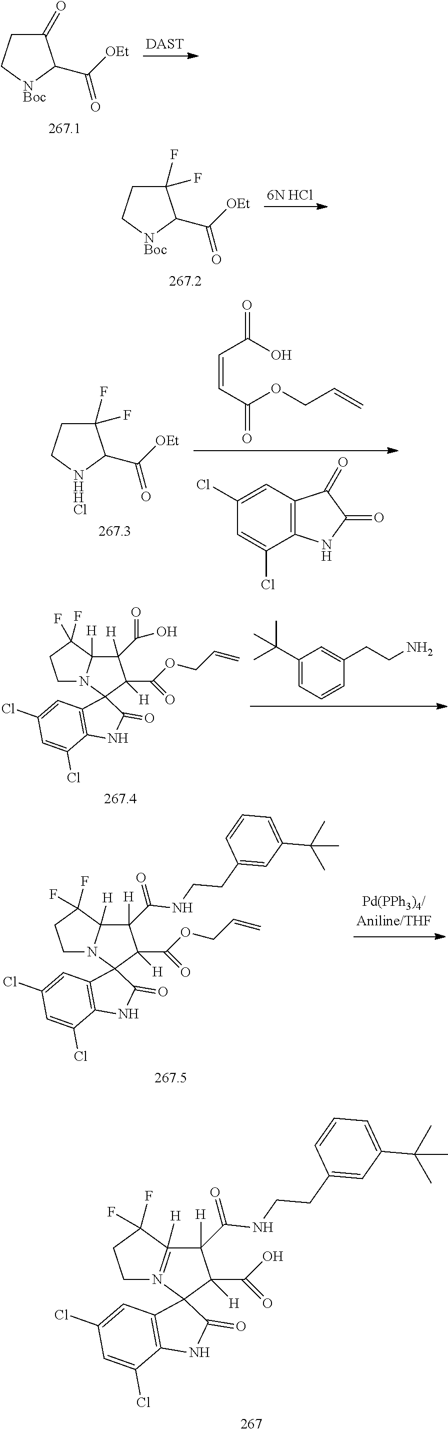

### Synthesis of 267.2

To a stirred solution of 267.1 (5.0 g, 19.4 mmol) in DCM (50 mL) was added DAST (7.8 mL, 58.3 mmol) dropwise at −78° C. After warming to RT and stirred for 16 h at RT, the reaction mixture was quenched with sat. NaHCO₃ solution at 0° C. and extracted with DCM (2×20 mL). The combined organic layer was dried over anhydrous Na₂SO₄, filtered and concentrated under reduced pressure. The residue was purified by flash chromatography (80 g Silica gel cartridge, 20%-30% EtOAc/pet ether) to afford 267.2 (5.0 g, 92%) as liquid. ¹H NMR (500 MHz, CDCl₃) (Exist in rotameric form): 4.44-4.40 (m, 1H), 4.30-4.23 (m, 2H), 3.78-3.75 (m, 1H), 3.57-3.55 (m, 1H), 2.60-2.30 (m, 2H), 1.48-1.42 (m, 9H), 1.33-1.29 (m, 3H).

### Synthesis of 267.3

To 267.2 (8 g, 28.6 mmol) was added 6N HCl (80 mL) at RT. After stirring at 65° C. for 6 h, the reaction mixture was concentrated under reduced pressure. The crude residue was washed 3 times with EtOAc:DCM (1:3; 40 mL) and dried under high vacuum to afford 267.3 (5.6 g, 73%) as a brown solid.

¹H NMR (400 MHz, D₂O): 4.64-4.58 (m, 1H), 3.66 (t, J=7.6 Hz, 2H), 2.74-2.62 (m, 2H).

### Synthesis of 267.4

To a solution of (Z)-4-(allyloxy)-4-oxobut-2-enoic acid (4.5 g, 28.8 mmol) in EtOH (50 mL) were added 267.3 (5.39 g, 28.8 mmol) and 5,7-dichloroindoline-2,3-dione (6.23 g, 28.8 mmol) at RT. After stirring at reflux for 2 h, the reaction mixture was concentrated. The residue was purified by flash chromatography (80 g Silica gel cartridge, 100% EtOAc) to afford 267.4 (4.2 g, 32%) as a brown solid.

LCMS: (28+32+10+6) %, m/z [M+H]⁺=461.0).

### Synthesis of 267.5

To a stirred solution of 267.4 (1.5 g, 3.25 mmol) in DMF (10 mL) were added DIPEA (1.7 mL, 9.75 mmol) and HATU (1.48 g, 3.90 mmol) at RT. After stirring for 10 minutes, 2-(3-(tert-butyl)phenyl)ethan-1-amine (0.86 g, 4.87 mmol) was added. After stirring for 3 h at RT, the reaction mixture was poured into ice cold water and stirred for 10 minutes. The resulting precipitate was filtered, washed with cold water (2×20 mL) and dried under vacuum (1.3 g). The residue was purified by prep. HPLC [Column: X-SELECT-C18 (150×19) mm, 5 μ; A: 10 mM Ammonium bicarbonate in H₂O, B:Acetonitrile; Gradient: (T%B): −0/50, 8/80, 10/80, 14/98, 16/98, 16.1/50, 18/50 at 18 mL/min] to afford major diastereomer (600 mg) which was further purified by flash chromatography (80 g Silica gel cartridge, 20%-30% EtOAc/pet ether) to afford 267.5 (200 mg, 10%) as a solid.

¹HNMR (400 MHz, DMSO-d₆): 11.20 (s, 1H), 8.57-8.54 (m, 1H), 7.58 (d, J=1.6 Hz, 1H), 7.23-7.14 (m, 3H), 7.07-7.01 (m, 2H), 5.33-5.24 (m, 1H), 5.06-5.02 (m, 2H), 4.21-4.16 (m, 2H), 4.01-3.96 (m, 3H), 3.67-3.61 (m, 2H), 2.72-2.50 (m, 4H), 2.50-2.33 (m, 2H), 1.27 (s, 9H); LCMS: 95.5%, m/z [M+H]⁺=620.0).

### Synthesis of 267

To a stirred solution of 267.5 (200 mg, 0.33 mmol) in THF (2 mL) were added aniline (30 mg, 0.32 mmol) and Pd(PPh₃)₄ (74 mg, 0.064 mmol) at RT. After stirring for 2 h at RT, the reaction mixture was concentrated under reduced pressure. The residue was purified by prep. HPLC [X BRIDGE-C18 (150×25), 5 μ; A: 0.1% Formic acid in H₂O, B: Acetonitrile; Gradient: (Time/%B): 0/60, 8/90, 8.1/98, 10/98, 10.1/60, 13/60 at 24 mL/min] to afford 267 (160 mg, 85%) as a solid.

¹H NMR (500 MHz, DMSO-d₆): 12.65 (br s, 1H), 11.13 (br s, 1H), 8.49 (br s, 1H), 7.57 (s, 1H), 7.23-7.18 (m, 3H), 7.11 (s, 1H), 7.07-7.01 (m, 1H), 4.02-3.90 (m, 2H), 3.60-3.56 (m, 1H), 3.32-3.22 (m, 2H), 2.71-2.67 (m, 2H), 2.50-2.22 (m, 4H), 1.27 (s, 9H); LCMS: 97.8%; m/z [M+H]⁺=580.1; absolute stereochemistry not known. Tested as racemate.

### Example 268

### Synthesis of 5,7-dichloro-1′-((3,5-dichlorophenyl)(methyl)carbamoyl)-7′,7′-difluoro-2-oxo-1′,2′,5′,6′,7′,7a′-hexahydrospiro[indoline-3,3′-pyrrolizine]-2′-carboxylic acid (268a & 268b):

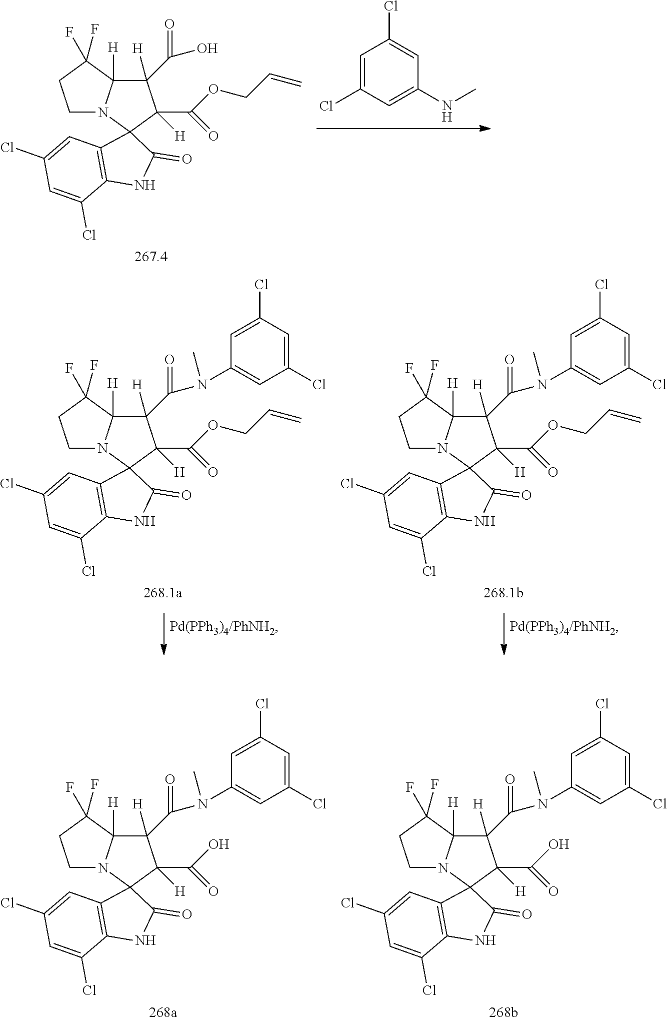

### Synthesis of 268.1a & 268.1b

To a stirred solution of 267.4 (500 mg, 1.08 mmol) in DCM (8 mL) were added 3,5-dichloro-N-methylaniline (230 mg, 1.30 mmol), TEA (0.36 mL, 2.61 mmol) and DMAP (39 mg, 0.32 mmol) at RT. After stirring for 30 min, POCl₃ (0.10 mL, 1.084 mmol) dissolved in DCM (1 mL) was added. After stirring for 16 h at RT, the reaction mixture was quenched with water (5 mL) and extracted with DCM (2×10 mL). The combined organic layer was dried over anhydrous Na₂SO₄, filtered and concentrated under reduced. The residue was purified by prep. HPLC [X-BRIDGE C18 (150×30) mm, 5 μ; A: 0.1% Formic acid in H₂O, B: Acetonitrile; Gradient: (Time/%B): 0/70, 8/90, 10/90, 10.1/98, 12/98, 12.1/70, 15/70 at 20 mL/min] to afford 268.1a (Peak-1, 390 mg, 58%) as a white solid and 268.1b (Peak-2, 140 mg, 21%) as a white solid.

268.1a: ¹H NMR (400 MHz, DMSO-d₆): 11.05 (br s, 1H), 7.90-7.44 (m, 5H), 5.80-5.64 (m, 1H), 5.25-4.90 (m, 2H), 4.90-4.75 (m, 1H), 4.39 (br s, 2H), 3.90-3.50 (m, 2H), 3.40-3.10 (m, 4H), 2.57-2.50 (m, 1H), 2.32-2.25 (m, 2H); LCMS: 95.8%, m/z [M+H]⁺=620.0.

268.1b: ¹H NMR (400 MHz, DMSO-d₆): 11.24 (s, 1H), 7.78 (s, 1H), 7.58-7.56 (m, 3H), 6.42 (s, 1H), 5.35-5.27 (m, 1H), 5.07-5.03 (m, 2H), 4.31-4.19 (m, 3H), 3.93-3.88 (m, 1H), 3.78-3.73 (m, 1H), 3.40-3.20 (m, 4H), 2.63-2.50 (m, 1H), 2.30-2.21 (m, 2H); LCMS: 86.9%, m/z [M+H]⁺=620.2.

### Synthesis of 268a

To a stirred solution of 268.1a (400 mg, 0.65 mmol) in THF (8 mL) were added aniline (60 μL, 0.65 mmol) and Pd(PPh₃)₄ (149 mg, 0.13 mmol) at RT. After stirring for 2 h at RT, the reaction mixture was concentrated under reduced pressure. The residue was purified by prep. HPLC [X-SELECT-C18 (150×19), 5 μ; A: 10 mM NH₄HCO₃ in H₂O, B: Acetonitrile; Gradient: (Time/%B): 0/20, 8/70, 8.1/98, 11/98, 11.1/20, 13/20 at 18 mL/min] to afford 268a (86 mg, 23%) as a solid.

¹H NMR (500 MHz, DMSO-d₆) (Exist in rotameric form): 12.90-12.70 (br s, 1H), 11.00-10.80 (br s, 1H), 7.61-6.50 (m, 5H), 4.84-4.79 (m, 1H), 4.60-4.30 (m, 1H), 3.85-3.68 (m, 1H), 3.32 (s, 3H), 3.30-3.10 (m, 1H), 2.60-2.50 (m, 1H), 2.40-2.15 (m, 2H); LCMS: 86.1%; m/z [M+H]⁺=578.0; absolute stereochemistry not known. Tested as racemate.

### Synthesis of 268b

To a stirred solution of 268.1b (150 mg, 0.24 mmol) in THF (3 mL) were added aniline (20 μL, 0.24 mmol) and Pd(PPh₃)₄ (55 mg, 0.05 mmol) at RT. After stirring for 2 h at RT, the reaction mixture was concentrated under reduced pressure. The residue was purified by prep. HPLC [KROMOSIL-C18 (150×25 mm), 10 μ; A: 0.1% Formic acid in H₂O, B: Acetonitrile; Gradient: (Time/%B): 0/50, 8/75, 9/75, 9.1/98, 11/98, 11.1/50, 13/50 at 23 mL/min] to afford 268b (50 mg, 35%) as a solid.

¹H NMR (500 MHz, DMSO-d₆): 13.01 (br s, 1H), 11.17 (s, 1H), 7.76-7.55 (m, 4H), 6.43 (s, 1H), 4.10 (d, J=11.0 Hz, 1H), 3.88-3.84 (m, 1H), 3.73-3.68 (m, 1H), 3.27 (s, 3H), 2.64-2.59 (m, 1H), 2.50-2.36 (m, 1H), 2.25-2.14 (m, 2H); LCMS: 99.3%, m/z [M+H]⁺=578.1; absolute stereochemistry not known. Tested as racemate.

### Example 269

### Synthesis of (269a & 269b)

### Synthesis of 269.2_1 and 269.2_2

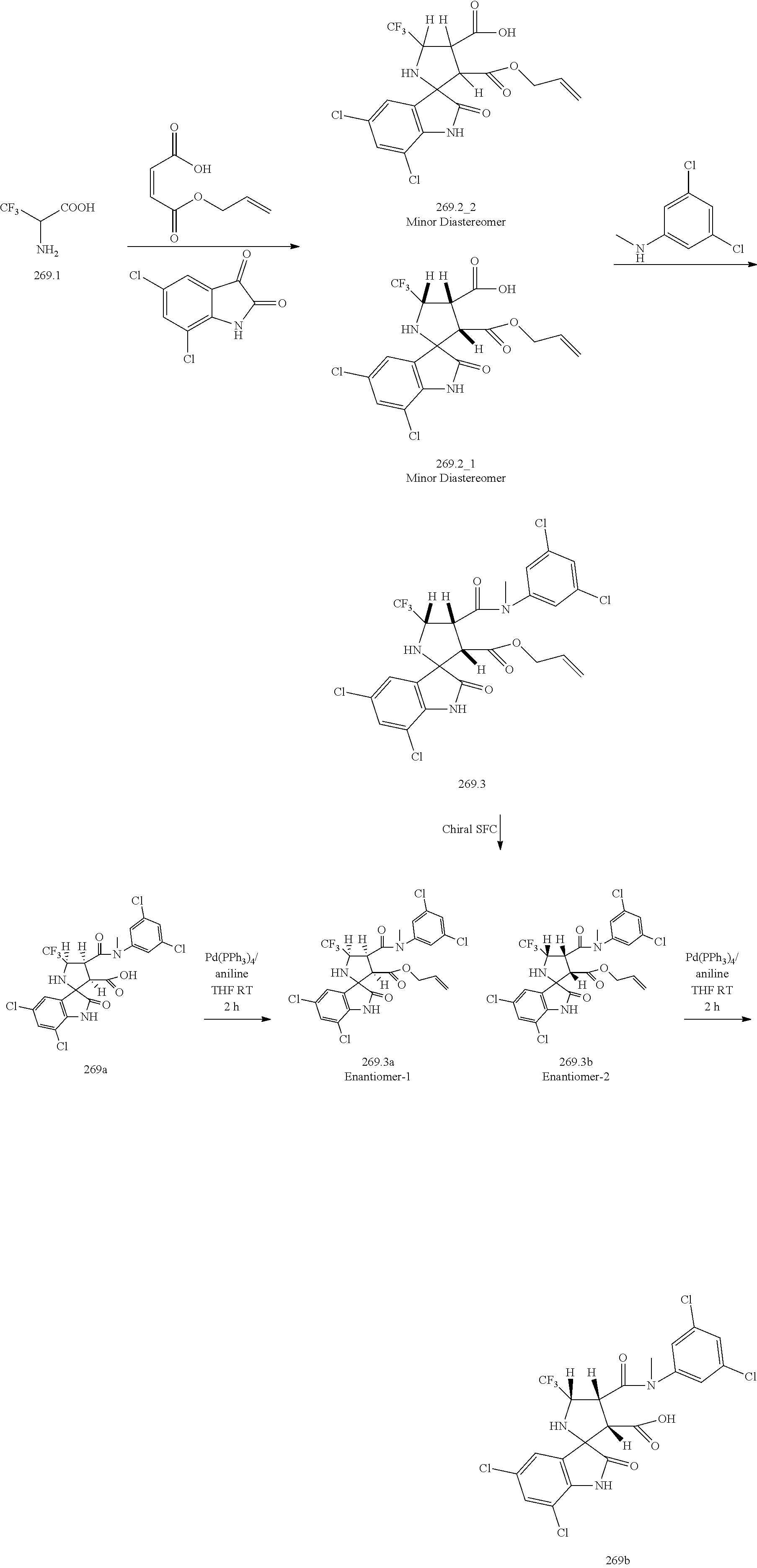

To a stirred solution of 269.1 (2 g, 13.9 mmol) in EtOH (70 mL) were added (Z)-4-(allyloxy)-4-oxobut-2-enoic acid (2.18 g, 13.9 mmol) and 5,7-dichloroindoline-2,3-dione (3 g, 13.9 mmol) at RT. After stirring for 3 h at 80° C., the reaction mixture was cooled to RT and concentrated under vacuum. The residue was purified by flash chromatography (80 g Silica gel cartridge, 40% EtOAc in pet ether) to afford major diastereomer 269.2_1 (750 mg, 12%) as a solid and minor diastereomer 269.2_2 (36 mg) as a solid.

269.2_1: ¹H NMR (500 MHz, DMSO-d₆): 12.78 (br s, 1H), 11.04 (s, 1H), 7.89 (d, J=2.0 Hz, 1H), 7.42 (d, J=2.5 Hz, 1H), 5.47-5.39 (m, 1H), 5.08-5.04 (m, 2H), 4.40-4.35 (m, 2H), 4.25 (d, J=5.5 Hz, 2H), 3.94 (d, J=7.5 Hz, 1H), 3.65 (t, J=7.0 Hz, 1H). Regio chemistry and relative stereochemistry was confirmed by 2D NMR studies.

269.2_2: ¹H NMR (500 MHz, DMSO-d₆): 12.65 (br s, 1H), 10.98 (br s, 1H), 7.98 (d, J=2.0 Hz, 1H), 7.42 (d, J=2.0 Hz, 1H), 5.96-5.89 (m, 1H), 5.42-5.37 (m, 2H), 4.65-4.57 (m, 2H), 4.42-4.38 (m, 2H), 3.88-3.71 (m, 1H), 3.69-3.65 (m, 1H); LCMS: 90.5%, m/z [M+H]⁺=453.2.

### Synthesis of 269.3

To a stirred solution of 269.2 (700 mg, 1.54 mmol) in pyridine (10 mL) was added EDC-HCl (592 mg, 3.09 mmol) at RT. After stirring for 10 minutes, 3,5-dichloro-N-methylaniline (408 mg, 2.31 mmol) was added. After stirring for 2 h at RT, the reaction mixture was evaporated under reduced pressure. The residue was purified by column chromatography (Silica gel 100-200 mesh, 20% EtOAc in pet ether) to afford 269.3 (180 mg, 19%) as a solid.

### Separation of 269.3a & 269.3b (Absolute Stereochemistry of Enantiomer 1 & 2 was not Determined)

269.3 (280 mg) was purified by chiral SFC (Chiralcel OX-H (30 ×250) mm, 5 μ; 50% CO₂: 50% Acetonitrile at RT (Isocratic 90 g/min, with detection at 214 nm). Pure fractions were concentrated under reduced pressure to give of 269.3a (Enantiomer-1, 90 mg, 64%) as a solid and of 269.3b (Enantiomer-2, 90 mg, 64%) as a solid.

269.3a: ¹H NMR (500 MHz, DMSO-d₆): 11.24 (s, 1H), 7.78 (br s, 1H), 7.61-7.55 (m, 2H), 7.49 (br s, 1H), 6.51 (br s, 1H), 5.33-5.28 (m, 1H), 5.06-5.03 (m, 2H), 4.57 (d, J=6.0 Hz, 1H), 4.24-4.18 (m, 3H), 3.85 (d, J=11.0 Hz, 1H), 3.76-3.72 (m, 1H), 3.25 (s, 3H); LCMS: 90.5%, m/z [M+H]⁺=611.9; Chiral purity: 99.8%.

269.3b: ¹H NMR (500 MHz, DMSO-d₆): 11.24 (s, 1H), 7.78 (br s, 1H), 7.66-7.55 (m, 2H), 7.49 (br s, 1H), 6.51 (br s, 1H), 5.33-5.28 (m, 1H), 5.06-5.03 (m, 2H), 4.57 (d, J=5.5 Hz, 1H), 4.24-4.18 (m, 3H), 3.85 (d, J=11.0 Hz, 1H), 3.77-3.72 (m, 1H), 3.26 (s, 3H); LCMS: 90.2%, m/z [M+H]⁺=611.9.

### Synthesis of 269a

To a stirred solution of 269.3a (80 mg, 0.13 mmol) in THF (3 mL) were added aniline (18 mg, 0.19 mmol) and Pd(PPh₃)₄ (30 mg, 0.026 mmol) at RT. After stirring for 2 h at RT, the reaction mixture was diluted with water (10 mL) and acidified with 1N HCl. The reaction mixture was extracted with EtOAc (20 mL). The organic layer was washed with brine (10 mL), dried over Na₂SO₄, filtered and evaporated. The residue was purified by prep. HPLC [Column: X BRIDGE-C18 (150×25), 5 μ; A: 0.1% Formic acid in H₂O, B: Acetonitrile; Gradient (T%B): −0/50, 8/80, 10/80, 10.1/98, 12/98, 12.1/50, 15/50 at 22 mL/min] to afford 269a (40 mg, 38%) as a solid.

¹H NMR (400 MHz, DMSO-d₆) (Exist in rotameric form): 13.08 (br s, 1H), 11.20 (br s, 1H), 7.76-7.32 (m, 4H), 6.53 (br s, 1H), 4.49-4.46 (m, 1H), 4.14-4.11 (m, 1H), 3.81-3.58 (m, 2H), 3.27 (s, 3H); LCMS: 99.4%, [M−H]⁻=567.9. (absolute stereochemistry was not determined).

### Synthesis of 269b

To a stirred solution of 269.3b (90 mg, 0.15 mmol) in THF (3 mL) were added aniline (21 mg, 0.22 mmol) and Pd(PPh₃)₄ (17 mg, 0.015 mmol) at RT. After stirring for 2 h at RT, the reaction mixture was diluted with water (10 mL) and acidified with 1N HCl. The reaction mixture was extracted with EtOAc (20 mL) and the orgaic layer was washed with brine (10 mL). The organic layer was dried over Na₂SO₄, filtered and concentrated under reduced pressure. The residue was purified by prep. HPLC [Column: X BRIDGE-C18 (150×25), 5 μ; A: 0.1% Formic acid in H₂O, B: Acetonitrile; Gradient (T%B): —0/50, 8/70, 10/70, 11.4/80, 11.15/98, 13/98, 13.1/50, 16/50 at 22 mL/min] to afford 269b (40 mg, 38%) as a sold.

¹H NMR (400 MHz, DMSO-d₆) (Exist in rotameric form): 13.04 (br s, 1H), 11.20 (br s, 1H), 7.76-7.33 (m, 4H), 6.52 (br s, 1H), 4.55-4.42 (m, 1H), 4.20-4.10 (m, 1H), 3.82-3.68 (m, 2H), 3.27 (s, 3H); LCMS: 99.0% [M−H]⁻=568.0. (absolute stereochemistry was not determined)

### Example 270

### Synthesis of (3′S,4′R,5′S)-5,7-dichloro-4′-((cyclopropylmethyl)(3,5-dichlorophenyl)carbamoyl)-2-oxo-5′-(trifluoromethyl)spiro[indoline-3,2′-pyrrolidine1-3′-carboxylic acid and (3′R,4′S,5′R)-5,7-dichloro-4′-((cyclopropylmethyl)(3,5-dichlorophenyl)carbamoyl)-2-oxo-5′-(trifluoromethyl)spiro[indoline-3,2′-pyrrolidine1-3′-carboxylic acid (270a & 270b)

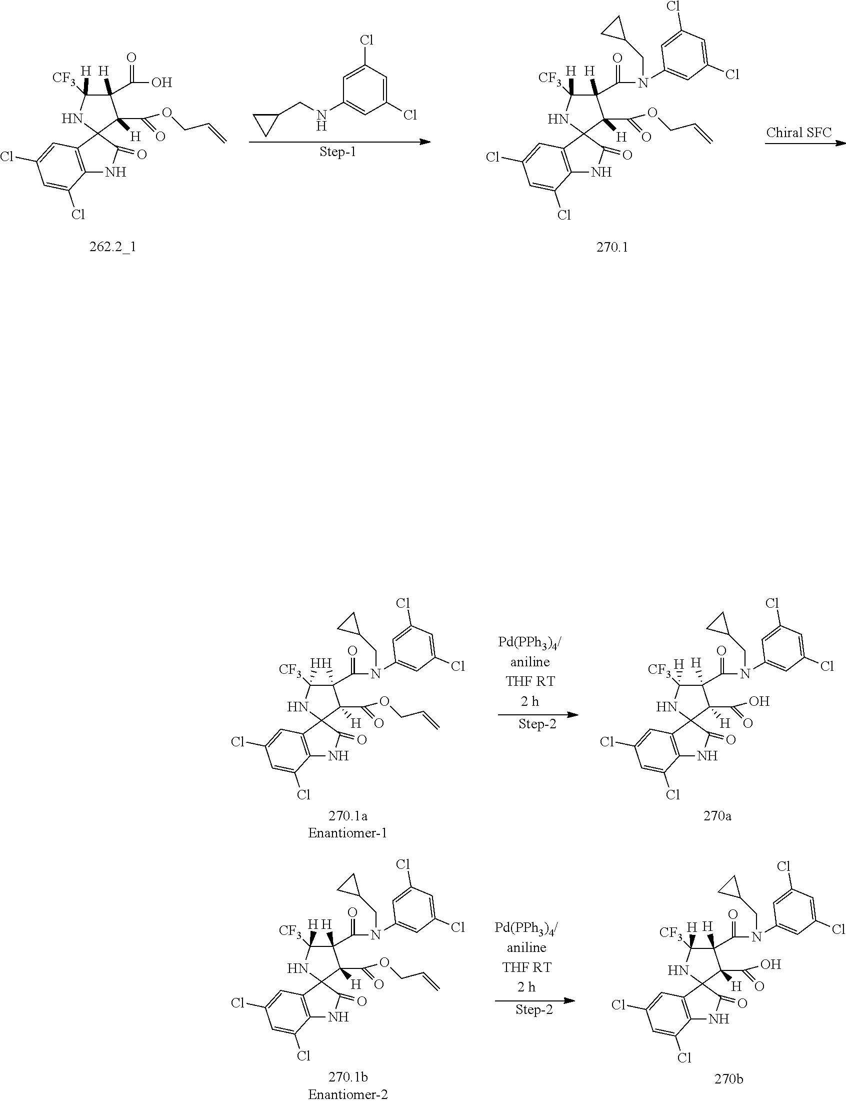

270a and 270b were synthesized from 269.2_1 following procedure described for the synthesis of 269a and 269b.

270a: ¹H NMR (500 MHz, DMSO-d₆) (Exist in rotameric form): 12.69 (br s, 1H), 11.04 (br s, 1H), 8.23/7.93 (d, J=1.5 Hz, 1H), 7.69-7.26 (m, 4H), 4.06-3.98 (m, 2H), 3.87 (d, J=7.0 Hz, 1H), 3.80-3.76 (m, 1H), 3.66 (t, J=6.5 Hz, 1H), 3.44-3.40 (m, 1H), 0.89-0.85 (m, 1H), 0.42-0.36 (m, 2H), 0.19-0.12 (m, 1H), 0.02-0.00 (m, 1H); LCMS: 98.1% [M+H]⁺=609.9; Chiral purity: 99.0%. Absolute stereo chemistry is unknown.

270b:

¹H NMR (500 MHz, DMSO-d₆) (Exist in rotameric form): 12.69 (br s, 1H), 11.04 (s, 1H), 8.23/7.93 (d, J=1.5 Hz, 1H), 7.69-7.26 (m, 4H), 4.06-3.98 (m, 2H), 3.87 (d, J=7.5 Hz, 1H), 3.80-3.76 (m, 1H), 3.66 (t, J=6.5 Hz, 1H), 3.44-3.40 (m, 1H), 0.89-0.85 (m, 1H), 0.42-0.37 (m, 2H), 0.19-0.16 (m, 1H), 0.03-0.00 (m, 1H); LCMS: 96.2% [M+H]⁺=609.9; Chiral purity: 97.6%. Absolute stereo chemistry is unknown.

### Example 271

### Synthesis of rac-(3′S,4′R,5′S)-5,7-dichloro-4′-((3,5-dichlorophenyl)(methyl)carbamoyl)-1-methyl-2-oxo-5′-(trifluoromethyl)spiro[indoline-3,2′-pyrrolidine]-3′-carboxylic acid (271):

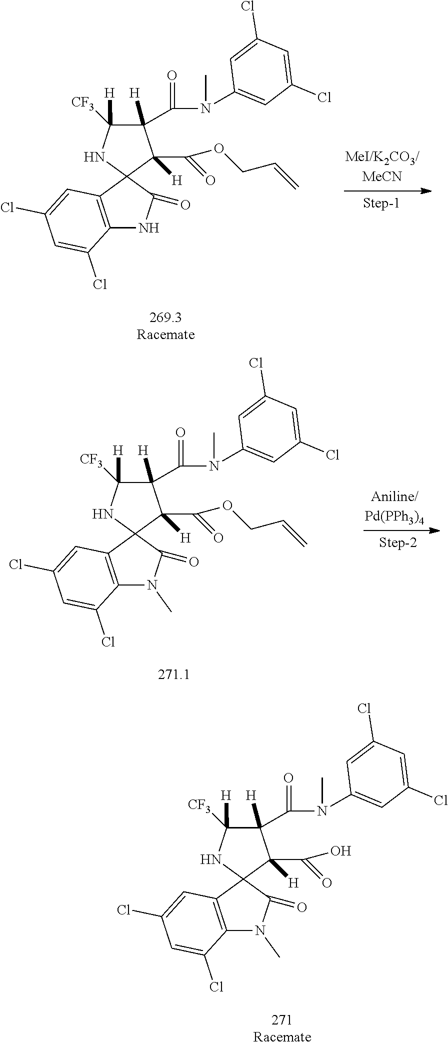 [SMILES: C=CCOC(=O)[C@H]1[C@@H](C(=O)N(C)c2cc(Cl)cc(Cl)c2)[C@@H](C(F)(F)F)NC12C(=O)Nc1c(Cl)cc(Cl)cc12 ; C=CCOC(=O)[C@H]1[C@@H](C(=O)N(C)c2cc(Cl)cc(Cl)c2)[C@@H](C(F)(F)F)NC12C(=O)N(C)c1c(Cl)cc(Cl)cc12 ; CN(C(=O)[C@H]1[C@@H](C(F)(F)F)NC2(C(=O)N(C)c3c(Cl)cc(Cl)cc32)[C@H]1C(=O)O)c1cc(Cl)cc(Cl)c1]

### Synthesis of 271.1

To a stirred solution of 269.3 (250 mg, 0.41 mmol) in acetonitrile (5 mL) was added K₂CO₃ (169 mg, 1.22 mmol) at RT. After stirring for 30 minutes at RT, MeI (0.05 mL, 0.81 mmol) was added. After stirring for 16 h at RT, the reaction mixture was quenched with water (25 mL) and extracted with EtOAc (50 mL). The organic layer was dried over Na₂SO₄, filtered and concentrated under reduced pressure. The residue was purified by silica gel column chromatography (Silica gel 100-200 mesh, 10% EtOAc in pet ether) to afford 271.1 (130 mg, 51%) as a brown solid.

¹H NMR (400 MHz, DMSO-d₆): 7.78 (br s, 1H), 7.66-7.58 (m, 2H), 7.52 (d, J=1.6 Hz, 1H), 7.36 (br s, 1H), 6.54 (br s, 1H), 5.39-5.32 (m, 1H), 5.13-5.05 (m, 2H), 4.53 (d, J=6.4 Hz, 1H), 4.27-4.15 (m, 3H), 3.90-3.80 (m, 1H), 3.78-3.69 (m, 1H), 3.44 (s, 3H), 3.26 (s, 3H); LCMS: 93.9%, m/z [M+H]⁺=626.00.

### Synthesis of 271

To a stirred solution of 271.1 (120 mg, 0.19 mmol) in THF (3 mL) were added aniline (21 mg, 0.23 mmol) and Pd(PPh₃)₄ (22 mg, 0.019 mmol) at RT. After stirring for 2 h at RT, the reaction mixture was diluted with water (10 mL) and acidified with 1N HCl. The reaction mixture was extracted with EtOAc (20 mL). The organic layer was washed with brine (10 mL), dried over Na₂SO₄, filtered and concentrated under reduced pressure. The residue was purified by prep. HPLC [Column: X-BRIDGE-C18 (150×30), 5 μ; A: 0.1% Formic acid in H₂O, B: Acetonitrile; Gradient: (T%B): −0/70, 8/90, 10/90, 10.1/98, 12/98, 12.1/70, 15/70 at 20 mL/min) to afford 271 (40 mg, 38%) as a solid.

¹H NMR (500 MHz, DMSO-d₆) (Exist in rotameric form): 13.11 (br s, 1H), 7.82-7.33 (m, 4H), 6.57 (s, 1H), 4.55-4.45 (m, 1H), 4.26-4.16 (m, 1H), 3.80 (d, J=11.5 Hz, 1H), 3.72-3.68 (m, 1H), 3.45 (s, 3H), 3.26 (s, 3H); LCMS: 96.6%, m/z [M+H]⁺=584.0;

### Example 272

### Synthesis of (272)

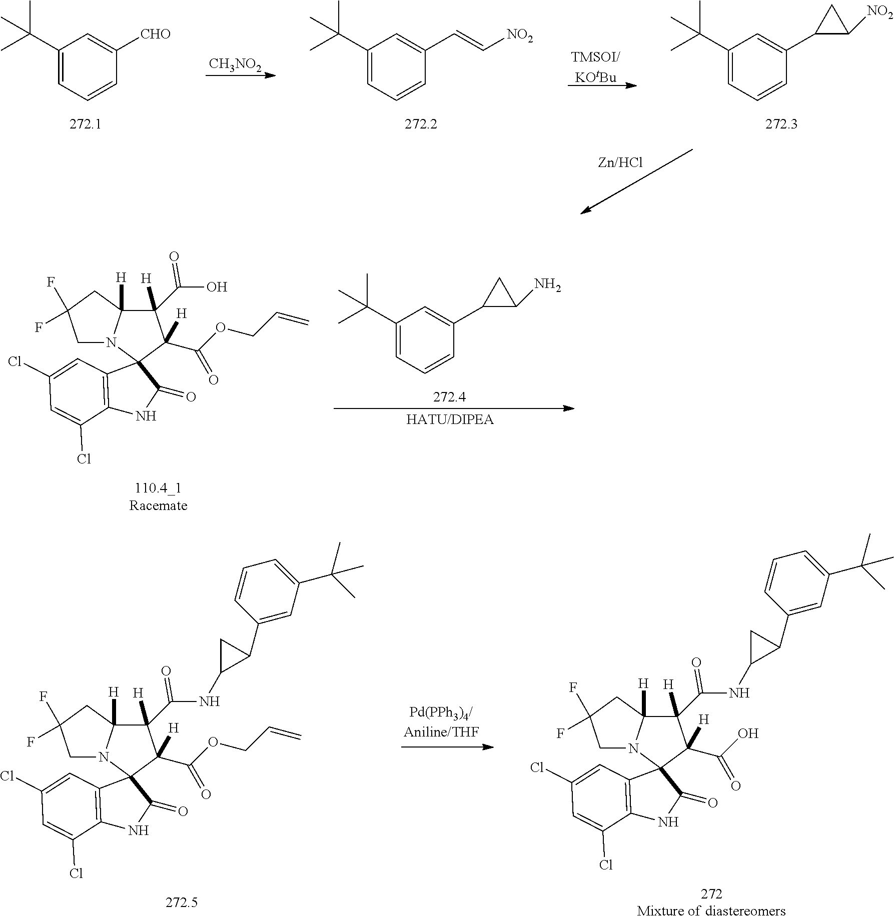

### Synthesis of 272.2

To a stirred solution of 272.1 (3.0 g, 18.5 mmol) in CH₃NO₂ (150 mL) was added NH₄OAc at RT. After stirring for 16 h at 120° C., the reaction mixture was cooled to RT and concentrated under reduced pressure. The residue was purified by column chromatography (Silica gel 100-200 mesh, 2% EtOAc in pet ether) to afford 272.2 (3.4 g, 89%) as liquid.

¹H NMR (500 MHz, CDCl₃): 8.03 (d, J=13.5 Hz, 1H), 7.60 (d, J=13.5 Hz, 1H), 7.55-7.54 (m, 2H), 7.41-7.37 (m, 2H), 1.35 (s, 9H); LCMS: 95.2%, m/z [M+H]⁺=206.2.

### Synthesis of 272.3

To a stirred solution of t-BuOK (4.09 g, 36.5 mmol) in DMSO (50 mL) was added TMSOI (8.03 g, 36.5 mmol) portion wise at RT. After stirring for 10 minutes, 272.2 (3.0 g, 14.6 mmol) was added dropwise at RT. After stirring for 30 minutes, the reaction mixture was poured into ice cold water and extracted with EtOAc (2×50 mL). The combined organic layer was washed with brine (25 mL), dried over anhydrous Na₂SO₄, filtered and concentrated under reduced pressure. The residue was purified column chromatography (Silica gel 100-200 mesh, 20% EtOAc in pet ether) to afford 272.3 (500 mg, 15%). LCMS: 82.8%, m/z [M+H]⁺=220.2.

### Synthesis of 272.4

To a stirred solution of 272.3 (140 mg, 0.63 mmol) in isopropanol (15 mL) were added 1N HCl (4.2 mL) and Zn (823 mg, 12.6 mmol) at RT. After stirring for 2 h, the reaction mixture was filtered through a small pad of Celite and the filtrate was concentrated under reduced pressure to afford 272.4 (110 mg) as a pale brown liquid which was used for the next step without purification. LCMS: 70.1%, m/z [M+H]⁺=190.3.

### Synthesis of 272.5

To a stirred solution of 110.4_1 (364 mg, 0.79 mmol) in DMF (5 mL) were added DIPEA (0.16 mL, 0.94 mmol), HATU (300 mg, 0.79 mmol) and 272.4 (150 mg, 0.79 mmol) at RT. After stirring at RT for 30 minutes. The reaction mixture was diluted with ice cold water. After stirring for 10 minutes, the resulting precipitate was filtered, washed with cold water and dried under vacuum to afford 272.5 (450 mg) which was used in the next step without purification. LCMS: 38.4%, m/z [M+H]⁺=632.1.

### Synthesis of 272

To a stirred solution of 272.5 (450 mg, 0.71 mmol) in THF (10 mL) were added aniline (79 mg, 0.85 mmol) and Pd(PPh₃)₄ (82 mg, 0.038 mmol) at RT. After stirring at RT for 2 h, the reaction mixture was diluted with water (10 mL) and pH adjusted to pH-6 with 1N HCl. The reaction mixture was extracted with EtOAc (2×20 mL). The combined organic layer was dried over anhydrous Na₂SO₄, filtered and concentrated under reduced pressure. The residue was purified by prep. HPLC [Column: X-BRIDGE-C18 (150×30), 5 μ; A: 0.1% Formic acid in H₂O, B: Acetonitrile; Gradient: (T%B): −0/50, 8/80, 10/80, 12/98, 14/98, 14.1/50, 17/50 at 20 mL/min] to afford mixture of diastereomers 272 (90 mg, 21%) as an off white solid.

¹H NMR (500 MHz, DMSO-d₆): 12.35 (br s, 1H), 10.98 (s, 1H), 8.51 (br s, 1H), 8.26 (d, J=6.5 Hz, 1H), 7.44-6.88 (m, 5H), 4.03-3.90 (m, 2H), 3.32-3.22 (m, 2H), 2.87-2.85 (m, 1H), 2.61-2.56 (m, 1H), 2.45-2.34 (m, 1H), 2.20-1.90 (m, 2H), 1.28 (s, 9H), 1.28-1.09 (m, 2H); LCMS: 95.48%, m/z [M+H]⁺=592.2.

### Example 273

### Synthesis of 1′-((4,6-bis(trifluoromethyl)pyridin-2-yl)(methyl)carbamoyl)-5,7-dichloro-6′,6′-difluoro-2-oxo-1′,2′,5′,6′,7′,7a′-hexahydrospiro[indoline-3,3′-pyrrolizine]-2′-carboxylic acid (273a & 273b)

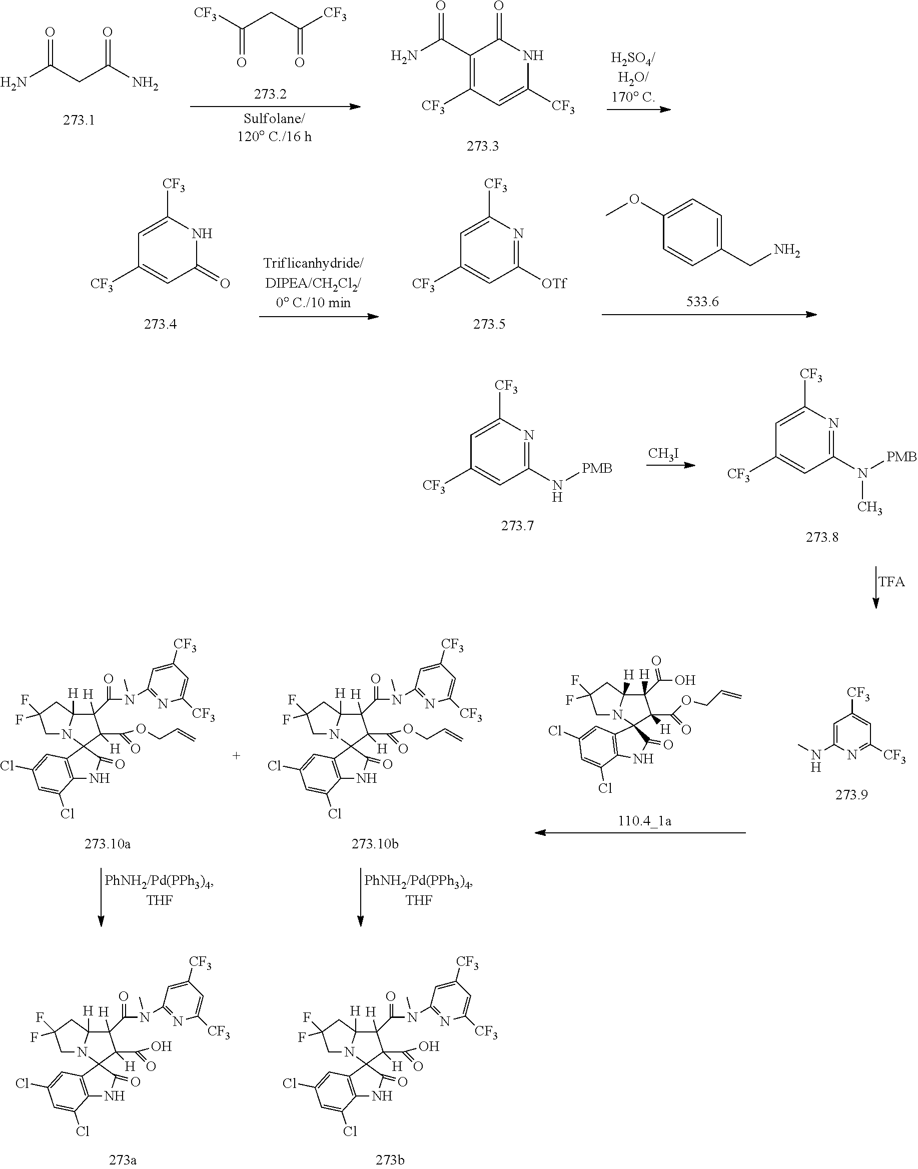

### Synthesis of 273.3

To a stirred solution of 273.1 (12.0 g, 118 mmol) in sulfolane (75 mL) was added 273.2 (24.4 g, 58.4 mmol) at RT. After stirring at 120° C. for 16 h, the reaction mixture was cooled to RT, poured into ice cold water and stirred well. The resulting precipitate was filtered, washed with cold water (2×20 mL) and dried under vacuum to afford 273.3 (18 g, 56%) as a white solid.

¹H NMR (500 MHz, DMSO-d₆): 13.03 (s, 1H), 8.00 (s, 1H), 7.83 (s, 1H), 7.62 (s, 1H); LCMS: 98.2%, m/z [M+H]⁺=275.0.

### Synthesis of 273.4

To 273.3 (36 g, 131 mmol) was added concentrated H2SO₄ (80 mL) and H₂O (54 mL) at RT. After stirring at 170° C. for 12 h, the reaction mixture was cooled to RT, poured into ice cold water and stirred well. The resulting precipitate was filtered, washed with cold water (2×50 mL) and dried under vacuum to afford 273.4 (20 g, 66%) as a white solid. LCMS: 99.6%, m/z [M+H]⁺=232.

### Synthesis of 273.5

To a stirred solution of 273.4 (20 g, 86.6 mmol) in DCM (200 mL) was added DIPEA (16 mL, 95.2 mmol) at 0° C. After stirring for 20 minutes, triflic anhydride (14.5 mL, 86.6 mmol) was dropwise added at 0° C. After stirring for 10 minutes at RT, the reaction mixture was quenched with water (50 mL) and the organic layer was separated. The aq. layer was extracted with DCM (50 mL). The combined organic layer was dried over anhydrous Na₂SO₄, filtered and concentrated under reduced pressure. The residue was purified by column chromatography (Silica gel 100-200 mesh; 2%-4% EtOAc/pet ether) to afford 273.5 (20 g, 64%) as solid. ¹H NMR (500 MHz, CDCl₃): 7.98 (s, 1H), 7.64 (s, 1H); ¹⁹F NMR (470 MHz, CDCl₃): −64.56, −68.26, −72.05.

### Synthesis of 273.7

To a stirred solution of 273.5 (10 g, 27.5 mmol) in THF (60 mL) was added DIPEA (9.6 mL, 55.1 mmol) at 0° C. After stirring for 30 minutes, 273.6 (4.15 g, 30.3 mmol) in THF (40 mL) was added at RT. After stirring for 16 h at RT, the reaction mixture was quenched with water (20 mL) and extracted with EtOAc (2×60 mL). The combined organic layer was dried over anhydrous Na₂SO₄, filtered and concentrated under reduced pressure. The residue was purified by flash column chromatography (80 g Silica gel cartridge; 10% EtOAc/pet ether) to afford 273.7 (8.5 g, 88%) as solid.

¹H NMR (500 MHz, CDCl₃): 7.29-7.26 (m, 2H), 7.09 (s, 1H), 6.90-6.87 (m, 2H), 6.71 (s, 1H), 4.50 (d, J=6.0 Hz, 2H), 3.81 (s, 3H); ¹⁹F NMR (470 MHz, CDCl₃): −65.35.−69.07; LCMS: 87.6%, m/z [M+H]⁺=351.0.

### Synthesis of 273.8

To a stirred solution of 273.7 (1.0 g, 2.85 mmol) in DMF (15 mL) were added dropwise NaHMDS (1M, 4.2 mL, 4.28 mmol) at 0° C. and MeI (0.18 mL, 2.85 mmol). After stirring for 2 h at RT, the reaction mixture was quenched with water (5 mL) and extracted with EtOAc (2×15 mL). The combined organic layer was dried over anhydrous Na₂SO₄, filtered and concentrated under reduced pressure. The residue was purified by flash column chromatography (24 g Silica gel cartridge; 20% EtOAc/pet ether) to afford 273.8 (0.85 g, 85%) as a solid.

¹H NMR (400 MHz, CDCl₃): 7.21 (d, J=8.4 Hz, 2H), 7.05 (s, 1H), 6.87-6.85 (m, 2H), 6.80 (s, 1H), 4.79 (s, 2H), 3.79 (s, 3H), 3.11 (s, 3H); LCMS: 94.3%, m/z [M+H]⁺=365.1.

### Synthesis of 273.9

To a stirred solution of 273.8 (0.700 g, 1.92 mmol) in DCM (5 mL) was added TFA (3.5 mL) at RT. After stirring for 1 h at RT, the reaction mixture was diluted with DCM (20 mL). The organic layer was collected, washed with saturated NaHCO₃ (2×10 mL) and water (10 mL), dried over anhydrous Na₂SO₄, filtered and concentrated under reduced pressure to afford 273.9 (0.35 g, 74%) as a solid.

¹H NMR (500 MHz, CDCl₃): 7.08 (s, 1H), 6.71 (s, 1H), 5.07 (br s, 1H), 3.01 (d, J=5.5 Hz, 3H).

### Synthesis of 273.10a & 273.10b

Thionyl chloride (5 mL) was added to 110.4_1a (0.500 g, 1.08 mmol) at RT. After stirring for 2 h, the reaction mixture was concentrated under reduced pressure to afford intermediate acid chloride. To this acid chloride in CH₂Cl₂ (5 mL) was added 273.9 (0.45 g, 1.87 mmol) in CH₂Cl₂ (5 mL). After stirring for 16 h at RT, the reaction mixture was quenched with water (10 mL). The organic layer was separated, and the aqueous layer was extracted with CH₂Cl₂ (2×15 mL). The combined organic layer was washed with brine (10 mL), dried over anhydrous Na₂SO₄, filtered and concentrated under reduced pressure. The residue was purified by prep. HPLC [Column: X-BRIDGE C18 (150×30), 5 μ; A: 0.1% Formic acid in H₂O, B: Acetonitrile; Gradient: (Time/%B): 0/70, 8/80, 10/98, 14/98, 16/98, 16.1/70, 20/70 at 23 mL/min] to afford 273.10a (Peak-1, 35 mg, 5%) as a white solid and 273.10b (Peak-2, 25 mg, 3%) as a white solid.

273.10a: ¹H NMR (400 MHz, DMSO-d₆): 11.22 (s, 1H), 8.38 (s, 1H), 8.27 (s, 1H), 7.55 (s, 1H), 7.33 (d, J=2.0 Hz, 1H), 5.30-5.21 (m, 1H), 5.02-4.98 (m, 2H), 4.24-4.11 (m, 3H), 4.09-3.99 (m, 3H), 3.60 (s, 3H), 2.91-2.72 (m, 2H), 2.54-2.50 (m, 1H); LCMS: 98.9%, m/z [M+H]⁺=687.2. Absolute stereochemistry was not determined.

273.10b: ¹H NMR (400 MHz, DMSO-d₆): 11.20-11.10 (br s, 1H), 8.52 (s, 1H), 8.26 (s, 2H), 7.50 (s, 1H), 5.45-5.39 (m, 1H), 5.06-5.02 (m, 2H), 4.22-3.90 (m, 5H), 3.50 (s, 3H), 3.22-3.12 (m, 1H), 2.69-2.67 (m, 1H), 2.15-2.09 (m, 2H); LCMS: 97.1%, m/z [M+H]⁺=687.2. Absolute stereochemistry was not determined.

### Synthesis of 273a

To a stirred solution of 273.10a (30 mg, 0.043 mmol) in THF (2 mL) were added aniline (4 μL, 0.043 mmol) and Pd(PPh₃)₄ (9 mg, 0.008 mmol) at RT. After stirring for 2 h at RT, the reaction mixture was concentrated under reduced pressure. The residue was purified by prep. HPLC [Column: KROMOSIL-C18 (150×25 mm), 10 μ; A: 0.1% Formic acid in H₂O, B: Acetonitrile; Gradient: (Time/%B): 0/20, 8/80, 10/80, 10.1/98, 12/98, 12.1/20, 14/20 at 20 mL/min] to afford 273a (14 mg, 50%) as solid.

¹H NMR (500 MHz, DMSO-d₆): 12.82 (br s, 1H), 11.15 (br s, 1H), 8.39 (br s, 1H), 8.28 (s, 1H), 7.55 (s, 1H), 7.27 (br s, 1H), 4.13-4.10 (m, 1H), 4.01-3.93 (m, 2H), 3.57 (s, 3H), 2.86-2.83 (m, 1H), 2.78-2.72 (m, 1H), 2.60-2.50 (m, 2H). LCMS: 99.3%, m/z [M+H]⁺=647.0; Chiral purity: 97.1%.

### Synthesis of 273b

To a stirred solution of 273.10b (20 mg, 0.029 mmol) in THF (2 mL) were added aniline (2 μL, 0.029 mmol) and Pd(PPh₃)₄ (6 mg, 0.005 mmol) at RT. After stirring for 2 h at RT, the reaction mixture was concentrated under reduced pressure. The residue was purified by prep. HPLC [Column: KROMOSIL-C18 (150×25 mm), 10 μ; A: 0.1% Formic acid in H₂O; B: Acetonitrile; Gradient: (Time/%B): 0/20, 8/80, 10/80, 10.1/98, 11/98, 11.1/40, 13/40 at 24 mL/min] to afford 273b (5 mg, 27%) as a solid. LCMS: 94.1%, m/z [M+H]⁺=647.0.

### Example 274

### Synthesis of (1′R,2′S,7a′R)-5,7-dichloro-1-((3,5-dichlorophenyl)(neopentyl)carbamoyl)-6′,6′-dimethyl-2-oxo1′,2′,5′,6′,7′,7a′-hexahydrospiro[indoline-3,3′-pyrrolizine]-2′-carboxylic acid and (1′S,2′R,7a′S)-5,7-dichloro-1′-((3,5-dichlorophenyl)(neopentyl)carbamoyl)-6′,6′-dimethyl-2-oxo-1′,2′,5′,6′,7′,7a′-hexahydrospiro[indoline-3,3′-pyrrolizine]-2′-carboxylic acid (274.6a & 274.6b):

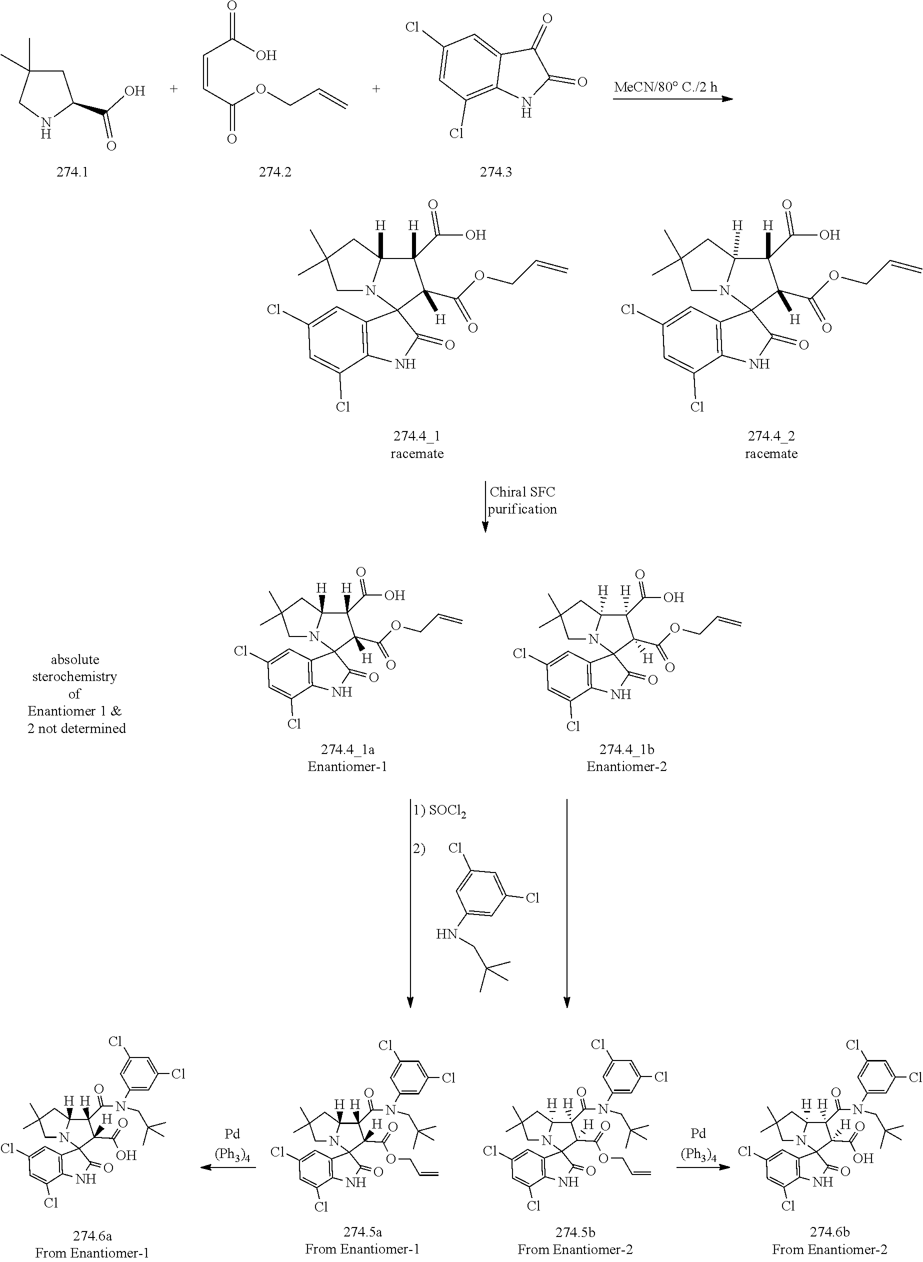

### Synthesis of 274.4_1 & 274.4_2

To a solution of 274.1 (5 g, 20.8 mmol) in MTBE (50 mL) were added 274.2 (3.2 g, 20.8 mmol) and 274.3 (4.4 g, 20.8 mmol) at RT. After stirring at 80° C. for 2 h, the reaction mixture was cooled to RT. The precipitate was filtered and washed with EtOAc. The filtrate was concentrated under reduced pressure. The residue was isolated by column chromatography (Silica-gel 100-200 mesh, 50% EtOAc/pet ether) to afford major diastereomer 274.4_1 (racemate, 1.65 g, 17%) as a white solid.

274.4_1: ¹H NMR (500 MHz, DMSO-d₆): 12.53 (s, 1H), 10.96 (s, 1H), 7.78 (d, J=2.0 Hz, 1H), 7.46 (d, J=2.0 Hz, 1H), 5.51-5.45 (m, 1H), 5.10-5.05 (m, 2H), 4.27-4.26 (m, 2H), 4.17-4.10 (m, 1H), 4.01 (d, J=7.5 Hz, 1H), 3.55 (t, J=7.5 Hz, 1H), 2.57 (d, J=7.5 Hz, 1H), 1.98 (d, J=7.0 Hz, 1H), 1.63-1.61 (m, 1H), 1.47-1.45 (m, 1H), 1.01 (s, 3H), 0.97 (s, 3H); LCMS: 99.1%, m/z [M+H]⁺=453.0; HPLC Purity: 98.9%; Chiral Purity: (49.8 +50.1)%. Regiochemistry and relative stereochemistry was confirmed by 2D NMR studies.

274.4_2: ¹H NMR (400 MHz, DMSO-d₆): 12.35 (br s, 1H), 10.90 (s, 1H), 7.55 (d, J=1.6 Hz, 1H), 7.50 (d, J=2.0 Hz, 1H), 5.87-5.80 (m, 1H), 5.27-5.14 (m, 2H), 4.45-4.37 (m, 3H), 3.74 (d, J=8.8 Hz, 1H), 3.46-3.42 (m, 1H), 2.51-2.48 (m, 1H), 2.08 (d, J=8.0 Hz, 1H), 1.92-1.87 (m, 1H), 1.60-1.55 (m, 1H), 1.02 (s, 6H); LCMS: 92.6%, m/z [M+H]⁺=453.1. Regiochemistry and relative stereochemistry was confirmed by 2D NMR studies.

### Separation of 274.4_1a & 274.4_1b

274.4_1 (1.6 g) was purified by chiral SFC using Lux Cellulose-4 (30×250) mm, 5 μ; 70% CO₂: 30% Isopropanol at RT (Isocratic 100 g/min, with detection at 214 nm). Pure fractions were concentrated under reduced pressure to give 274.4_1a (Peak-1, 625 mg, 78%) as a white solid and 274.4_1b (Peak-2, 650 mg, 81%) as a solid. (absolute stereochemistry of Enantiomer 1 & 2 not determined).

274.4_1a: ¹H NMR (500 MHz, DMSO-d₆): 12.50 (s, 1H), 10.96 (s, 1H), 7.78 (d, J=2.0 Hz, 1H), 7.46 (d, J=2.0 Hz, 1H), 5.51-5.45 (m, 1H), 5.10-5.05 (m, 2H), 4.28-4.26 (m, 2H), 4.17-4.10 (m, 1H), 4.01 (d, J=8.0 Hz, 1H), 3.55 (t, J=7.5 Hz, 1H), 2.57 (d, J=7.5 Hz, 1H), 1.98 (d, J=7.5 Hz, 1H), 1.63-1.61 (m, 1H), 1.48-1.45 (m, 1H), 1.01 (s, 3H), 0.97 (s, 3H); LCMS: 99.2%, m/z [M+H]⁺=453.1; Chiral Purity: 99.7%.

274.4_1b: ¹H NMR (500 MHz, DMSO-d₆): 12.53 (s, 1H), 10.96 (s, 1H), 7.79 (d, J=1.5 Hz, 1H), 7.46 (d, J=2.0 Hz, 1H), 5.49-5.46 (m, 1H), 5.11-5.05 (m, 2H), 4.28-4.27 (m, 2H), 4.14-4.13 (m, 1H), 4.00 (d, J=6.5 Hz, 1H), 3.55 (t, J=7.5 Hz, 1H), 2.57 (d, J=7.5 Hz, 1H), 1.97 (d, J=7.5 Hz, 1H), 1.64-1.61 (m, 1H), 1.48-1.44 (m, 1H), 1.01 (s, 3H), 0.97 (s, 3H); LCMS: 98.9%, m/z [M+H]⁺=453.1; Chiral Purity: 99.7%.

### Synthesis of 274.5a

Thionyl chloride (6 mL) was added to 274.4_1a (300 mg, 0.66 mmol) at RT. After stirring for 2 h at RT, the reaction mixture was concentrated under reduced pressure to afford intermediate acid chloride. To this acid chloride in CH₂Cl₂ (5 mL) was added a solution of 3,5-dichloro-N-neopentylaniline (296 mg, 1.27 mmol) in CH₂Cl₂ (5 mL). After stirring at 55° C. for 16 h, the reaction mixture was concentrated under reduced pressure. The residue was purified by flash column chromatography (40 g Silica gel cartridge, 20% to 30% EtOAc/pet ether) to afford 274.5a (230 mg, 53%) as solid.

¹H NMR (500 MHz, DMSO-d₆) (Exist in rotameric form): 10.90/10.84 (s, 1H), 8.26 (s, 1H), 7.63 (s, 1H), 7.44-7.39 (m, 3H), 5.48-5.39 (m, 1H), 5.09-5.05 (m, 2H), 4.27-4.13 (m, 3H), 3.84-3.68 (m, 3H), 3.54 (t, J=7.5 Hz, 1H), 2.68 (d, J=7.0 Hz, 1H), 1.86 (d, J=7.0 Hz, 1H), 1.54-1.49 (m, 2H), 0.97 (s, 6H), 0.84/0.82 (s, 9H); LCMS: 98.8%, m/z [M+H]⁺=668.0.

### Synthesis of 274.6a

To a stirred solution of 274.5a (230 mg, 0.34 mmol) in THF (10 mL) were added aniline (48 mg, 0.68 mmol) and Pd(PPh₃)₄ (80 mg, 0.06 mmol) at RT. After stirring for 2 h at RT, the reaction mixture was concentrated under reduced pressure. The residue was purified by prep. HPLC [Column: X-BRIDGE-C18 (150×30) mm, 5 μ; A: 0.1% Formic acid in H₂O, B: Acetonitrile; Gradient: (Time/%B): 0/70, 8/90, 10/70, 10.1/98, 12/98, 12.1/70, 15/70 at 20 mL/min) to afford 274.6a (118 mg, 55%) as a solid.

¹H NMR (500 MHz, DMSO-d₆) (Exist in rotameric form): 12.40 (br s, 1H), 10.85/10.78 (s, 1H), 8.41/7.91 (d, J=2.0 Hz, 1H), 7.61/7.52 (t, J=2.0 Hz, 1H), 7.47-7.39 (m, 3H), 4.07 (d, J=8.0 Hz, 1H), 3.94 (d, J=14.0 Hz, 1H), 3.84-3.78 (m, 1H), 3.51 (d, J=14.0 Hz, 1H), 3.45 (t, J=7.0 Hz, 1H), 2.68 (d, J=7.0 Hz, 1H), 1.82 (d, J=7.0 Hz, 1H), 1.50-1.41 (m, 2H), 0.96 (s, 6H), 0.84/0.82 (s, 9H); LCMS: 99.3%, m/z [M+H]⁺=626.0; Chiral purity: 99.8%.

### Synthesis of 274.5b

Thionyl chloride (5 mL) was added to 274.4_1b (100 mg, 0.22 mmol) at RT. After stirring for 2 h at RT, the reaction mixture was concentrated under reduced pressure to afford acid chloride intermediate. To this acid chloride in CH₂Cl₂ (5 mL) was added a solution of 3,5-dichloro-N-neopentylaniline (98 mg, 0.42 mmol) in CH₂Cl₂ (5 mL). After stirring at 55° C. for 16 h, the reaction mixture was concentrated under reduced pressure. The residue was purified by flash column chromatography (40 g Silica gel cartridge, 20% to 30% EtOAc/pet ether) to afford 274.5b (100 mg, 68%) as a white solid.

¹H NMR (400 MHz, DMSO-d₆) (Exist in rotameric form): 10.90/10.84 (s, 1H), 8.26/7.78 (d, J=2.0 Hz, 1H), 7.63/7.54 (t, J=2.0 Hz, 1H), 7.44-7.39 (m, 3H), 5.48-5.39 (m, 1H), 5.09-5.04 (m, 2H), 4.30-4.12 (m, 3H), 3.85-3.67 (m, 3H), 3.54 (t, J=7.6 Hz, 1H), 2.68-2.66 (m, 1H), 1.86 (d, J=6.8 Hz, 1H), 1.54-1.49 (m, 2H), 0.97 (s, 6H), 0.84/0.81 (s, 9H); LCMS: 96.0%, m/z [M+H]⁺=667.9.

### Synthesis of 274.6b

To a stirred solution of 274.5b (100 mg, 0.15 mmol) in THF (5 mL) were added aniline (17 mg, 0.18 mmol) and Pd(PPh₃)₄ (34.6 mg, 0.03 mmol) at RT. After stirring for 3 h at RT, the reaction mixture was concentrated under reduced pressure. The residue was purified by prep. HPLC [Column: KROMOSIL-C18 (150×25) mm, 10 μ; A: 0.1% Formic acid in H₂O, B: Acetonitrile; Gradient: (Time/%B): 0/70, 8/95, 10/98, 12/98, 12.1/70, 14/70 at 25 mL/min] to afford 274.6b (65 mg, 69%) as a solid.

¹H NMR (400 MHz, DMSO-d₆) (Exist in rotameric form): 12.38 (br s, 1H), 10.80/10.77 (s, 1H), 8.41/7.92 (d, J=2.0 Hz, 1H), 7.60/7.52 (t, J=1.6 Hz, 1H), 7.47-7.38 (m, 3H), 4.06 (d, J=8.0 Hz, 1H), 3.93 (d, J=14.0 Hz, 1H), 3.84-3.77 (m, 1H), 3.52 (d, J=14.0 Hz, 1H), 3.45 (t, J=7.2 Hz, 1H), 2.68 (d, J=6.8 Hz, 1H), 1.82 (d, J=6.8 Hz, 1H), 1.48-1.43 (m, 2H), 0.96 (s, 6H), 0.84/0.82 (s, 9H); LCMS: 99.0%, m/z [M+H]⁺=626.0; Chiral purity: 99.4%.

### Example 275

### Synthesis of (5′R,6′S,7′R,7a′R)-5″,7″-dichloro-7′-((3,5-dichlorophenyl)(neopentyl)carbamoyl)-2″-oxo-1′,6′,7′,7a′-tetrahydro-3′H-dispiro[cyclopropane-1,2′-pyrrolizine-5′,3″-indoline1-6′-carboxylic acid and (5′S,6′R,7′S,7a′S)-5″,7″-dichloro-7′-((3,5-dichlorophenyl)(neopentyl)carbamoyl)-2″-oxo-1′,6′,7′,7a′-tetrahydro-3′H-dispiro]cyclopropane-1,2′-pyrrolizine-5′,3″-indoline]-6′-carboxylic acid (275.7a & 275.7b)

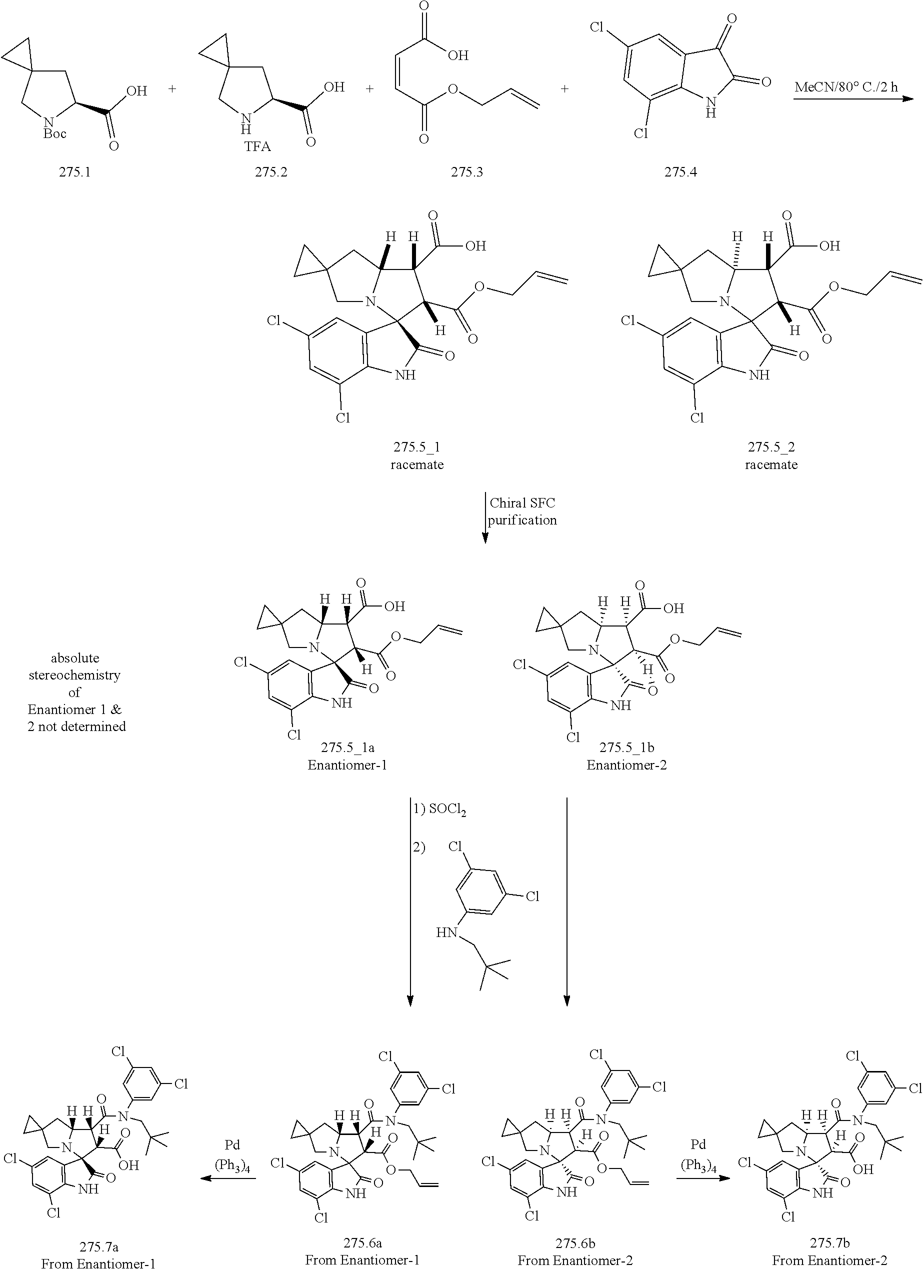

### Synthesis of 275.2

To a stirred solution of 275.1 (10 g, 41.4 mmol) in DCM (100 mL) was added TFA (16 mL, 207 mmol) at 0° C. After stirring at RT for 16 h, the reaction mixture was concentrated under reduced pressure. The resulting residue was triturated with diethyl ether (200 mL) to afford 275.2 (7.4 g, 75%) as a solid.

¹H NMR (400 MHz, DMSO-d₆): 9.47 (br s, 1H), 4.43 (dd, J=8.4 Hz, J=6.8 Hz, 1H), 3.16 (d, J=11.2 Hz, 2H), 3.12 (d, J=11.2 Hz, 2H), 2.24-2.18 (m, 1H), 2.03-1.98 (m, 1H), 0.68-0.61 (m, 4H); LCMS: 87.6%, m/z [M+H]⁺=142.2.

### Synthesis of 275.5_1 & 275.5_2

To a solution of 275.2 (4.2 g, 17.6 mmol) in MTBE (60 mL) were added 275.3 (2.75 g, 17.6 mmol) and 275.4 (3.81 g, 17.6 mmol) at RT. After stirring at 80° C. for 2 h, the reaction mixture was cooled to RT. The resulting precipitate was filtered and washed with EtOAc. The filtrate was concentrated under reduced pressure. The residue was purified by column chromatography (Silica gel 100-200 mesh, 10%-30% EtOAc/pet ether). The major diastereomer was triturated with DCM (50 mL) to afford 275.5_1 (racemate, 3.0 g, 38%) as a white solid.

¹H NMR (500 MHz, DMSO-d₆): 12.60 (br s, 1H), 11.03 (s, 1H), 7.74 (d, J=2.0 Hz, 1H), 7.46 (d, J=2.0 Hz, 1H), 5.51-5.48 (m, 1H), 5.11-5.06 (m, 2H), 4.28 (d, J=5.5 Hz, 2H), 4.22-4.16 (m, 1H), 4.04 (d, J=7.5 Hz, 1H), 3.63-3.58 (m, 1H), 2.75 (d, J=7.5 Hz, 1H), 2.04 (d, J=8.0 Hz, 1H), 1.93-1.85 (m, 1H), 1.52-1.49 (m, 1H), 0.50-0.36 (m, 4H); LCMS: 96.8%, m/z [M+H]⁺=451.1; Chiral Purity: (49.9%+50.0%). The regio and relative stereochemistry was confirmed by 2D NMR analysis.

275.5_2: ¹H NMR (400 MHz, DMSO-d₆): 12.45 (br s, 1H), 10.98 (s, 1H), 7.58 (d, J=1.6 Hz, 1H), 7.51 (d, J=2.0 Hz, 1H), 5.89-5.79 (m, 1H), 5.28-5.15 (m, 2H), 4.51-4.37 (m, 3H), 3.65-3.57 (m, 2H), 2.72 (d, J=8.8 Hz, 1H), 2.04-1.99 (m, 2H), 1.77-1.72 (m, 1H), 0.65-0.35 (m, 4H); LCMS: 91.9%, m/z [M+H]⁺=451.0. The regiochemistry and relative stereochemistry was confirmed by 2D NMR analysis.

### Separation of 275.5_1a & 275.5_1b

275.5_1 (3 g) was purified by chiral SFC using Chiralpak AD-H (30×250) mm, 5 μ; A: 80% CO₂%, B: 20% of 0.5% TFA in Isopropanol at RT (Isocratic 100 g/min, with detection at 214 nm). Pure fractions were concentrated under reduced pressure to give 275.5_1a (Peak-1, 1.2 g, 80%) as a solid and 275.5_1b (Peak-2, 1.4 g, 93%) as a solid. Absolute stereochemistry of Enantiomer 1 & 2 not determined.

275.5_1a: ¹H NMR (500 MHz, DMSO-d₆): 12.60 (br s, 1H), 11.10 (s, 1H), 7.71 (d, J=1.5 Hz, 1H), 7.49 (d, J=2.0 Hz, 1H), 5.53-5.49 (m, 1H), 5.13-5.07 (m, 2H), 4.31 (d, J=5.5 Hz, 2H), 4.26-4.24 (m, 1H), 4.05 (d, J=7.5 Hz, 1H), 3.69-3.63 (m, 1H), 2.88-2.82 (m, 1H), 2.09 (d, J=8.0 Hz, 1H), 1.97-1.90 (m, 1H), 1.55-1.52 (m, 1H), 0.52-0.36 (m, 4H); LCMS: 98.3%, m/z [M+H]⁺=451.0; Chiral Purity: 99.1%.

275.5_1b: ¹H NMR (500 MHz, DMSO-d₆): 12.65 (br s, 1H), 11.10 (s, 1H), 7.71 (s, 1H), 7.49 (s, 1H), 5.55-5.48 (m, 1H), 5.12-5.07 (m, 2H), 4.31 (d, J=5.5 Hz, 2H), 4.27-4.23 (m, 1H), 4.05 (d, J=8.0 Hz, 1H), 3.69-3.66 (m, 1H), 2.85 (d, J=7.0 Hz, 1H), 2.09 (d, J=8.0 Hz, 1H), 1.97-1.93 (m, 1H), 1.55-1.52 (m, 1H), 0.51-0.37 (m, 4H); LCMS: 98.0%, m/z [M+H]⁺=451.0; Chiral Purity: 95.4%.

### Synthesis of 275.6a

Thionyl chloride (5 mL) was added to 275.5_1a (300 mg, 0.66 mmol) at RT. After stirring for 2 h, the reaction mixture was concentrated under reduced pressure to afford acid chloride intermediate. To this acid chloride in CH₂Cl₂ (5 mL) was added a solution of 3,5-dichloro-N-neopentylaniline (296 mg, 1.27 mmol) in CH₂Cl₂ (5 mL). After stirring at 55° C. for 16 h, the reaction mixture was concentrated under reduced pressure. The residue was purified by flash column chromatography (40 g Silica gel cartridge, 20% to 30% EtOAc/pet ether) to afford 275.6a (300 mg, 71%) as solid.

¹H NMR (400 MHz, DMSO-d₆) (Exist in rotameric form): 10.91/10.98 (s, 1H), 8.21/7.71 (d, J=1.6 Hz, 1H), 7.63/7.54 (t, J=1.6 Hz, 1H), 7.43-7.39 (m, 3H), 5.67-5.46 (m, 1H), 5.23-5.08 (m, 2H), 4.34-4.19 (m, 3H), 4.14 (d, J=8.0 Hz, 1H), 3.89-3.80 (m, 2H), 3.68-3.60 (m, 2H), 2.80 (d, J=7.6 Hz, 1H), 1.93-1.88 (m, 1H), 1.43-1.39 (m, 1H), 0.83/0.81 (s, 9H), 0.47-0.33 (m, 4H); LCMS: 98.2%, m/z [M+H]⁺=665.9.

### Synthesis of 275.7a

To a stirred solution of 275.6a (300 mg, 0.44 mmol) in THF (10 mL) wereadded aniline (50 mg, 0.53 mmol) and Pd(PPh₃)₄ (104 mg, 0.08 mmol) at RT. After stirring for 2 h at RT, the reaction mixture was concentrated under reduced pressure. The residue was purified by prep. HPLC [Column: KROMOSIL-C18 (150×25) mm, 10 μ; A: 0.1% Formic Acid in H₂O, B: Acetonitrile; Gradient: (Time/%B): 0/70, 8/95, 10/98, 12/98, 12.1/70, 14/70 at 25 mL/min] to afford 275.7a (113 mg, 40%) as a solid.

¹H NMR (400 MHz, DMSO-d₆) (Exist in rotameric form): 12.43/12.30 (br s, 1H), 10.90/10.85 (s, 1H), 8.37/7.87 (d, J=2.0 Hz, 1H), 7.61/7.53 (t, J=2.0 Hz, 1H), 7.48 (d, J=1.6 Hz, 2H), 7.42/7.39 (d, J=2.0 Hz, 1H), 4.07 (d, J=8.0 Hz, 1H), 4.00 (d, J=14.0 Hz, 1H), 3.88-3.86 (m, 1H), 3.55 (t, J=7.2 Hz, 1H), 3.44 (d, J=14.0 Hz, 1H), 2.87 (d, J=7.2 Hz, 1H), 1.87-1.82 (m, 2H), 1.36-1.33 (m, 1H), 0.83/0.82 (s, 9H), 0.45-0.32 (m, 4H); LCMS: 99.0%, m/z [M+H]⁺=624.0; Chiral purity: 98.3%.

### Synthesis of 275.6b

Thionyl chloride (5 mL) was added to 275.5_1b (300 mg, 0.66 mmol) at RT. After stirring for 2 h, the reaction mixture was concentrated under reduced pressure to afford acid chloride intermediate. To this acid chloride in CH₂Cl₂ (5 mL) was added a solution of 3,5-dichloro-N-neopentylaniline (296 mg, 1.27 mmol) in CH₂Cl₂ (5 mL). After stirring at 55° C. for 16 h, the reaction mixture was concentrated under reduced pressure. The residue was purified by flash column chromatography (40 g Silica gel cartridge, 20% to 30% EtOAc/pet ether) to afford 275.6b (200 mg, 45%) as solid.

LCMS: 92.9%, m/z [M+H]⁺=666.1.

### Synthesis of 275.7b

To a stirred solution of 275.6b (200 mg, 0.30 mmol) in THF (10 mL) were added aniline (56 mg, 0.60 mmol) and Pd(PPh₃)₄ (104 mg, 0.08 mmol) at RT. After stirring for 3 h at RT, the reaction mixture was concentrated under reduced pressure. The residue was purified by prep. HPLC [Column: KROMOSIL-C18 (150×25) mm, 10 μ, A: 0.1% Formic Acid in H₂O, B: Acetonitrile; Gradient: (Time/%B): 0/70, 8/95, 10/98, 12/98, 12.1/70, 14/70 at 25 mL/min] to afford 275.7b (30 mg, 16%) as a solid.

¹H NMR (400 MHz, DMSO-d₆) (Exist in rotameric form): 12.42/12.30 (br s, 1H), 10.90/10.85 (s, 1H), 8.37/7.86 (d, J=2.0 Hz, 1H), 7.61/7.53 (t, J=2.0 Hz, 1H), 7.48 (d, J=1.6 Hz, 2H), 7.42/7.39 (d, J=2.0 Hz, 1H), 4.07 (d, J=7.6 Hz, 1H), 4.00 (d, J=14.0 Hz, 1H), 3.88-3.86 (m, 1H), 3.55 (t, J=7.2 Hz, 1H), 3.44 (d, J=14.0 Hz, 1H), 2.87 (d, J=7.6 Hz, 1H), 1.89-1.82 (m, 2H), 1.36-1.32 (m, 1H), 0.83/0.82 (s, 9H), 0.45-0.32 (m, 4H); LCMS: 95.8%, m/z [M+H]⁺=624.0; Chiral purity: 95.8%.

### Example 280

### Synthesis of (1′R,2′R,7a′R)-5,7-dichloro-1-((cyclopropylmethyl)(3,5-dichlorophenyl)carbamoyl)-6′,6′-difluoro-2-oxo-1′,2′,5′,6′,7′,7a′-hexahydrospiro[indoline-3,3′-pyrrolizine]-2′-carboxylic acid

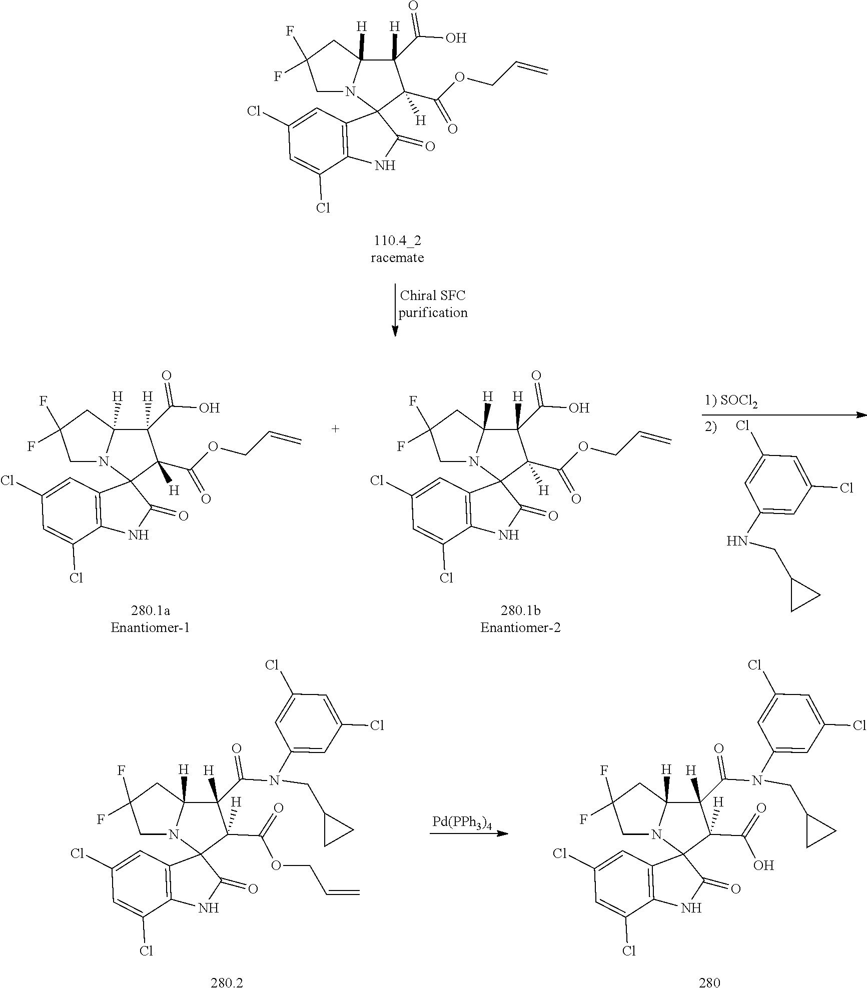

280 was synthesized from 280.1b following the procedure described for the synthesis of 110. Absolute stereochemistry is unknown.

¹H NMR (500 MHz, DMSO-d₆) (Exist in rotameric form): 12.66 (br s, 1H), 10.91 (br s, 1H), 7.92-7.38 (m, 5H), 4.62-4.55 (m, 1H), 3.67-3.59 (m, 1H), 3.48-3.38 (m, 2H), 3.20-3.07 (m, 2H), 2.74-2.64 (m, 1H), 2.50-2.41 (m, 1H), 2.30-2.15 (m, 1H), 0.90-0.80 (m, 1H), 0.38-0.36 (m, 2H), 0.06-0.04 (m, 2H); LCMS: 92.6%, m/z [M+H]⁺=618.0; Chiral Purity: 95.1%.

### Example 281

### Synthesis of (1′S,2′S,7a′S)-5,7-dichloro-1-((cyclopropylmethyl)(3,5-dichlorophenyl)carbamoyl)-6′,6′-difluoro-2-oxo-1′,2′,5′,6′,7′,7a′-hexahydrospiro[indoline-3,3′-pyrrolizine]-2′-carboxylic acid

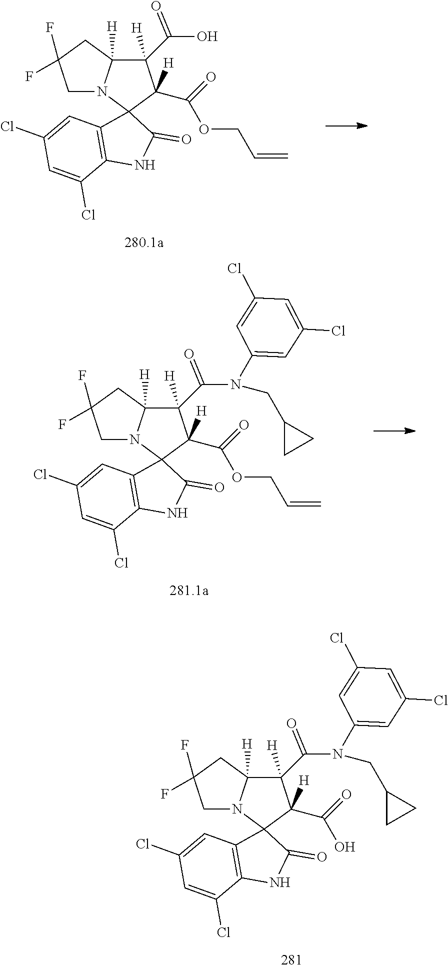 [SMILES: C=CCOC(=O)[C@H]1[C@H](C(=O)O)[C@@H]2CC(F)(F)CN2C12C(=O)Nc1c(Cl)cc(Cl)cc12 ; C=CCOC(=O)[C@H]1[C@H](C(=O)N(CC2CC2)c2cc(Cl)cc(Cl)c2)[C@@H]2CC(F)(F)CN2C12C(=O)Nc1c(Cl)cc(Cl)cc12 ; O=C(O)[C@H]1[C@H](C(=O)N(CC2CC2)c2cc(Cl)cc(Cl)c2)[C@@H]2CC(F)(F)CN2C12C(=O)Nc1c(Cl)cc(Cl)cc12]

281 was synthesized from 110.4_2 following the procedure described for the synthesis of 111. Absolute stereochemistry is unknown.

¹H NMR (500 MHz, DMSO-d₆) (Exist in rotameric form): 12.66 (br s, 1H), 10.91 (br s, 1H), 7.92-7.38 (m, 5H), 4.61-4.53 (m, 1H), 3.68-3.58 (m, 1H), 3.49-3.39 (m, 2H), 3.22-3.07 (m, 2H), 2.72-2.65 (m, 1H), 2.50-2.40 (m, 1H), 2.30-2.15 (m, 1H), 0.90-0.81 (m, 1H), 0.38-0.36 (m, 2H), 0.06-0.04 (m, 2H); LCMS: 90.3%, m/z [M+H]⁺=618.0.

### Example 282

### Synthesis of methyl (1′R,2′S,7a′R)-5,7-dichloro-1-((3,5-dichlorophenyl)(methyl)carbamoyl)-6′,6′-difluoro-2′-methyl-2-oxo1′,2′,5′,6′,7′,7a′-hexahydrospiro [indoline-3,3′-pyrrolizine]-2′-carboxylate

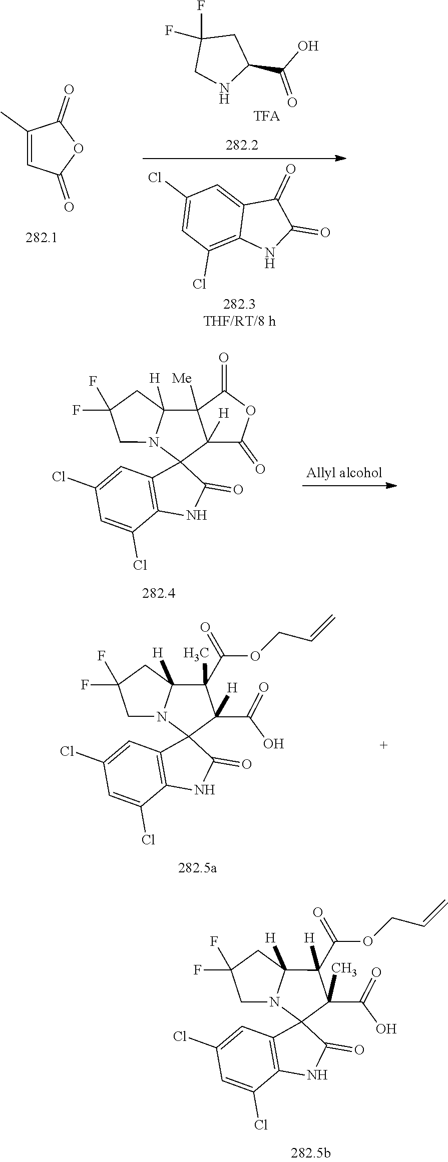

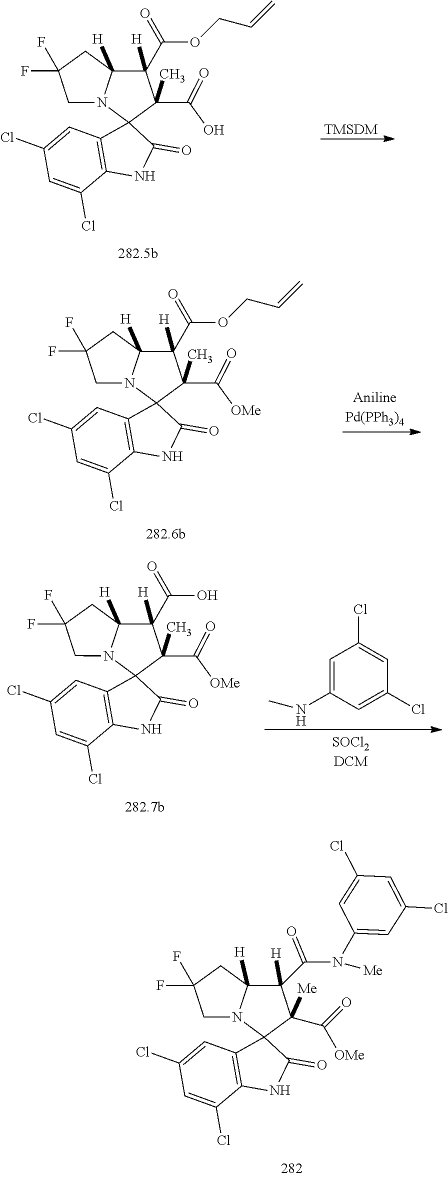

### Synthesis of 282.4

To a stirred solution of 282.1 (2.5 g, 22.3 mmol) in THF (25 mL) were added 282.2 (5.5 g, 22.3 mmol) and 282.3 (4.8 g, 22.3 mmol) at RT. After stirring at RT for 8 h, the reaction mixture was evaporated under reduced pressure to afford 282.4 (8.5 g) as a brown solid, which was used as such in the next step without purification. LCMS: 16%, m/z [M+H]⁺=416.9.

### Synthesis of 282.5a and 282.5b

A solution of 282.4 (8.5 g, 20.4 mmol) in allyl alcohol (50 mL) was heated at 90° C. for 16 h. The reaction mixture was concentrated under reduced pressure. The residue was purified by flash column chromatography (120 g Silica gel cartridge, 30% EtOAc/pet ether) to afford mixture of regio-isomers. The regio isomers were purified by prep. HPLC to obtain 282.5a (160 mg, 2%) as an off-white solid and 282.5b (1.1 g, 11%) as an off-white solid. The regio chemistry and relative stereochemistry was confirmed by 2D NMR analysis.

282.5a data: ¹H NMR (400 MHz, MeOH-d4): 7.89 (d, J=1.6 Hz, 1H), 7.29 (d, J=1.6 Hz, 1H), 5.56-5.47 (m, 1H), 5.11-5.05 (m, 2H), 4.35-4.24 (m, 2H), 3.82-3.78 (m, 1H), 3.74 (s, 1H), 3.07-2.98 (m, 1H), 2.73-2.64 (m, 1H), 2.38-2.32 (m, 1H), 2.19-2.10 (m, 1H), 1.59 (s, 3H); LCMS: 95.5%, m/z [M+H]⁺=475.0.

282.5b: ¹H NMR (500 MHz, DMSO-d₆): 13.40 (br s, 1H), 11.10 (br s, 1H), 7.61 (s, 1H), 7.22 (s, 1H), 5.95-5.87 (m, 1H), 5.34 (dd, J=17.5 Hz, J=1.5 Hz, 1H), 5.23 (dd, J=10.5 Hz, J=1.5 Hz, 1H), 4.62-4.54 (m, 2H), 4.45-4.42 (m, 1H), 4.02 (d, J=10.5 Hz, 1H), 3.88-3.72 (m, 1H), 3.20-2.98 (m, 1H), 2.80-2.75 (m, 1H), 2.31-2.26 (m, 1H), 1.30 (s, 3H); LCMS: 91.5%, m/z [M+H]⁺=475.0.

### Synthesis of 282.6b

To a stirred solution of 282.5b (400 mg, 0.84 mmol) in methanol (8 mL) was added trimethyl silyldiazomethane (2M in hexane, 2.1 mL, 4.2 mmol) at 0° C. After stirring at RT for 16 h, the reaction mixture was cooled to 0° C. and quenched with acetic acid (0.5 mL). After stirring at RT for 1 h, the reaction mixture was concentrated under reduced pressure at low temperature. The residue was purified by flash column chromatography (24 g Silica gel cartridge, 10% to 25% EtOAc/pet ether) to obtain 282.6b (260 mg, 63%) as an off-white solid.

¹H NMR (400 MHz, DMSO-d₆): 11.31 (s, 1H), 7.67 (d, J=2.0 Hz, 1H), 6.91 (d, J=2.0 Hz, 1H), 5.94-5.86 (m, 1H), 5.37-5.25 (m, 2H), 4.59 (d, J=4.8 Hz, 2H), 4.54-4.47 (m, 1H), 4.11 (d, J=10.4 Hz, 1H), 3.77-3.69 (m, 1H), 3.64 (s, 3H), 2.91-2.81 (m, 2H), 2.40-2.33 (m, 1H), 1.24 (s, 3H); LCMS: 91.0%, m/z [M−H]⁻=486.9.

### Synthesis of 282.7b

To a stirred solution of 282.6b (240 mg, 0.49 mmol) in THF (5 mL) were added aniline (46 mg, 0.49 mmol) and Pd(PPh₃)₄ (114 mg, 0.09 mmol) at RT. After stirring for 4 h at RT, the reaction mixture was concentrated under reduced pressure. The residue was purified by column chromatography (Silica gel 100-200 mseh, 30% EtOAc/pet ether) to afford 282.7b (120 mg, 55%) as a yellow solid.

¹H NMR (400 MHz, DMSO-d₆): 12.90 (br s, 1H), 11.28 (s, 1H), 7.66 (d, J=1.6 Hz, 1H), 6.90 (d, J=2.0 Hz, 1H), 4.49-4.42 (m, 1H), 4.06-3.97 (m, 2H), 3.82-3.71 (m, 1H), 3.64 (s, 3H), 2.97-2.84 (m, 1H), 2.39-2.30 (m, 1H), 1.19 (s, 3H); LCMS: 95.1%, m/z [M+H]⁺=450.4.

### Synthesis of 282

Thionyl chloride (1 mL) was added to 282.7b (70 mg, 0.15 mmol) at RT. After stirring for 2 h, the reaction mixture was concentrated under reduced pressure to afford intermediate acid chloride. To this acid chloride in CH₂Cl₂ (2 mL) were added a solution of 3,5-dichloro-N-methylaniline (38 mg, 0.21 mmol) in CH₂Cl₂ (3 mL) and catalytic amount of DMAP (2 mg) at RT. After stirring at 55° C. for 16 h, the reaction mixture was concentrated under reduced pressure. The reside was purified by flash column chromatography (12 g Silica gel cartridge, 15% to 20% EtOAc/pet ether) to afford 282 (50 mg, 57%) as an off-white solid.

¹H NMR (500 MHz, DMSO-d₆) (Exist in rotameric form): 11.31/11.13 (s, 1H), 7.77-7.44 (m, 4H), 6.99-6.91 (m, 1H), 4.40/4.33 (d, J=10.0 Hz, 1H), 3.96-3.72 (m, 1H), 3.65/3.58 (s, 3H), 3.46-3.39 (m, 1H), 3.34/3.17 (s, 3H), 2.88-2.77 (m, 2H), 2.17-2.07 (m, 1H), 1.10/1.04 (s, 3H); LCMS: 94.5%, m/z [M+H]⁺=605.9.

### Example 284

### Synthesis of (1′R,2′S,3R,7a′R)-5,7-dichloro-N1′-(3,5-dichlorophenyl)-6′,6′-difluoro-N1′-neopentyl-2-oxo-1′,2′,5′,6′,7′,7a′-hexahydrospiro[indoline-3,3′-pyrrolizine]-1′,2′-dicarboxamide:

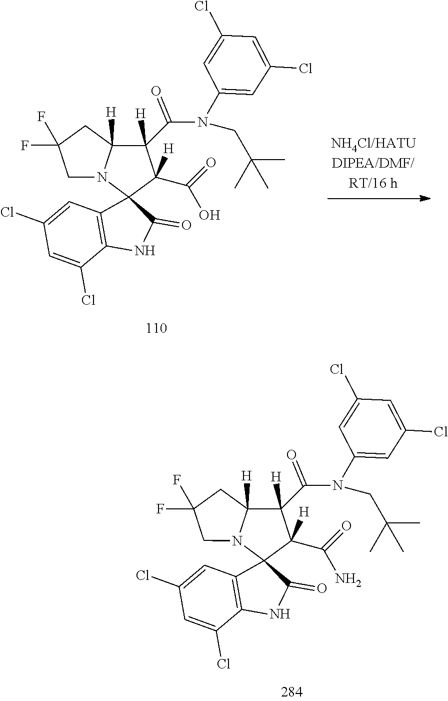 [SMILES: CC(C)(C)CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1 ; CC(C)(C)CN(C(=O)[C@H]1[C@H]2CC(F)(F)CN2[C@]2(C(=O)Nc3c(Cl)cc(Cl)cc32)[C@H]1C(N)=O)c1cc(Cl)cc(Cl)c1]

To a stirred solution of 110 (300 mg, 0.47 mmol) in DMF (6 mL) were added HATU (360 mg, 0.94 mmol) and DIPEA (0.51 mL, 2.83 mmol) at room temperature. After stirring for 10 minutes, NH₄Cl (128 mg, 2.36 mmol) was added. After stirring for 16 h at RT, the reaction mixture was diluted with ice-cold water (50 mL) and stirred for 10 minutes. The resulting precipitate was filtered, washed with water and dried under vacuum. The crude compound was purified by prep. HPLC [KROMOSIL-C18 (150×25) mm, 10 μ; A: 0.1% Formic acid in H₂O, B: Acetonitrile; Gradient: (Time/%B): 0/55, 8/80, 8.1/98, 10/98, 10.1/55, 13/55 at 20 mL/min] to afford 284 (70 mg, 23%) as an off-white solid.

¹H NMR (400 MHz, DMSO-d₆) (Exist in rotameric form): 10.80/10.70 (s, 1H), 7.70 (t, J=1.6 Hz, 1H), 7.67 (d, J=1.6 Hz, 1H), 7.61-7.54 (m, 2H), 7.49 (d, J=1.6 Hz, 1H), 6.95 (br s, 1H), 6.42 (br s, 1H), 4.33-4.27 (m, 1H), 3.92 (d, J=11.6 Hz, 1H), 3.78 (d, J=14.0 Hz, 1H), 3.68-3.58 (m, 2H), 3.45-3.35 (m, 1H), 2.81-2.76 (m, 1H), 2.50-2.44 (m, 1H), 2.28-2.13 (m, 1H), 0.81 (s, 9H); LCMS: 99.1%, m/z [M+H]⁺=633.0; Chiral purity: 94.7%.

### Example 285

### Synthesis of (1′R,2′S,3R,7a′R)-5,7-dichloro-N1′-(3,5-dichlorophenyl)-6′,6′-difluoro-N2′-hydroxy-N2′-methyl-N1′-neopentyl-2-oxo-1′,2′,5′,6′,7′,7a′-hexahydrospiro[indoline-3,3′-pyrrolizine]-1′,2′-dicarboxamide

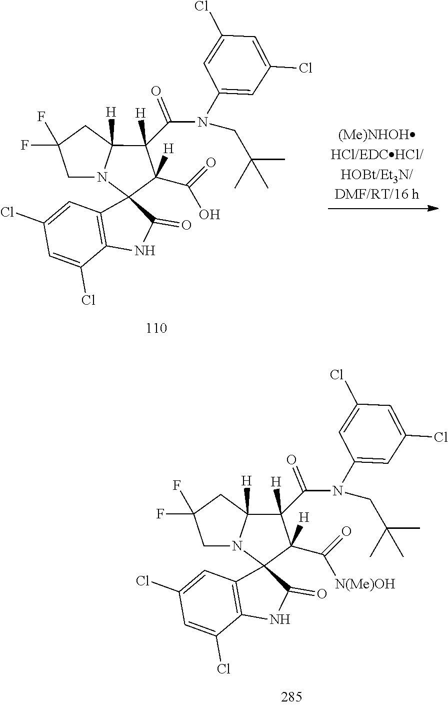

To a stirred solution of 110 (400 mg, 0.62 mmol) in DMF (5 mL) were added EDC.HCl (240 mg, 1.25 mmol), HOBt (255 mg, 1.88 mmol) and Et₃N (0.88 mL, 6.29 mmol) at room temperature. After stirring for 5 minutes, (Me)NHOH.HCl (420 mg, 5.03 mmol) was added. After stirring for 16 h at RT, the reaction mixture was diluted with ice-cold water (50 mL) and stirred for 10 minutes. The resulting precipitate was filtered, washed with water and dried under vacuum. The crude compound was purified by prep. HPLC [X-SELECT-C18 (250×19) mm, 5 μ; A: 10 mM Ammonium bicarbonate in H₂O, B: Acetonitrile; Gradient: (Time/%B): 0/65, 8/80, 10/80, 10.1/98, 12/98, 12.1/65, 15/65 at 20 mL/min] to afford 285 (53 mg, 13%) as an off-white solid.

¹H NMR (500 MHz, DMSO-d₆): 10.77 (s, 1H), 10.10 (s, 1H), 7.67 (s, 1H), 7.59-7.44 (m, 3H), 7.28 (s, 1H), 4.40 (d, J=8.0 Hz, 1H), 4.15-4.08 (m, 1H), 3.78 (d, J=14.0 Hz, 1H), 3.63 (d, J=14.0 Hz, 1H), 3.52 (t, J=9.0 Hz, 1H), 3.44-3.40 (m, 1H), 2.98 (s, 3H), 2.71-2.66 (m, 1H), 2.64-2.55 (m, 1H), 2.21-2.10 (m, 1H), 0.82 (s, 9H); LCMS: 97.2%, m/z [M+H]⁺=662.9; Chiral purity: 93.2%.

### Example 286

### Synthesis of 5,7-dichloro-N1′-(3,5-dichlorophenyl)-6′,6′-difluoro-N2′-hydroxy-N1′-neopentyl-2-oxo-1′,2′,5′,6′,7′,7a′-hexahydrospiro[indoline-3,3′-pyrrolizine]-1′,2′-dicarboxamide (286a & 286b)

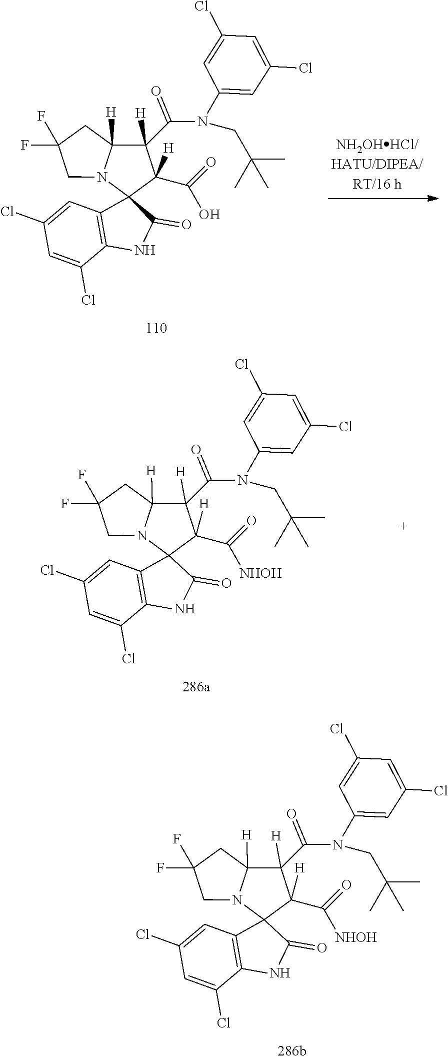

To a stirred solution of 110 (400 mg, 0.62 mmol) in DMF (5 mL) were added HATU (360 mg, 0.94 mmol) and DIPEA (1.2 mL, 6.29 mmol) at RT. After stirring for 10 minutes, NH₂OH.HCl (352 mg, 5.03 mmol) was added. After stirring for 16 h at RT, the reaction mixture was diluted with ice cold water (50 mL) and stirred for 15 minutes. The resulting precipitate was filtered, washed with water (10 mL) and dried under vacuum. The residue was purified by prep. HPLC [X-SELECT-C18 (150×30) mm, 5 μ; A: 10 mM Ammonium bicarbonate in H₂O, B: Acetonitrile; Gradient: (Time/%B): 0/60, 8/90, 11/90, 11.1/98, 15/98, 15.1/60, 19/60 at 22 mL/min] to obtain 286a (Peak-1, 37 mg, 9%) as an off-white solid and 286b (Peak-2, 38 mg, 9%) as an off-white solid.

286a: ¹H NMR (500 MHz, DMSO-d₆): 10.74 (br s, 1H), 9.96 (br s, 1H), 8.83 (s, 1H), 7.71-7.52 (m, 5H), 4.32 (t, J=10.5 Hz, 1H), 3.80-3.75 (m, 1H), 3.71-3.63 (m, 3H), 3.38-3.32 (m, 1H), 2.87-2.83 (m, 1H), 2.64-2.50 (m, 1H), 2.23-2.16 (m, 1H), 0.80 (s, 9H); LCMS: 97.0%, m/z [M+H]⁺=648.9; chiral purity: 99.8%.

286b: LCMS: 95.9%, m/z [M+H]⁺=649.0.

### Example 287

### Synthesis of 1′-(((3s,5s,7s)-adamantan-1-yl)(methyl)carbamoyl)-5,7-dichloro-6′,6′-difluoro-2-oxo-1′,2′,5′,6′,7′,7a′-hexahydrospiro[indoline-3,3′-pyrrolizine]-2′-carboxylic acid

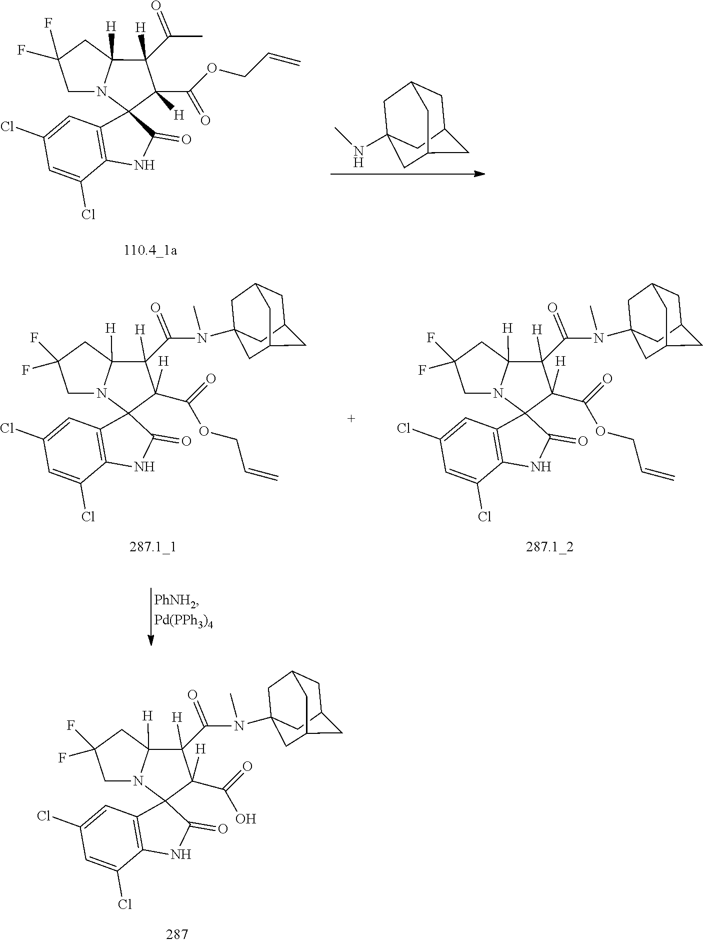 [SMILES: C=CCOC(=O)[C@H]1[C@@H](C(C)=O)[C@H]2CC(F)(F)CN2[C@]12C(=O)Nc1c(Cl)cc(Cl)cc12 ; CNC12CC3CC(CC(C3)C1)C2 ; C=CCOC(=O)C1C(C(=O)N(C)C23CC4CC(CC(C4)C2)C3)C2CC(F)(F)CN2C12C(=O)Nc1c(Cl)cc(Cl)cc12 ; C=CCOC(=O)C1C(C(=O)N(C)C23CC4CC(CC(C4)C2)C3)C2CC(F)(F)CN2C12C(=O)Nc1c(Cl)cc(Cl)cc12 ; CN(C(=O)C1C2CC(F)(F)CN2C2(C(=O)Nc3c(Cl)cc(Cl)cc32)C1C(=O)O)C12CC3CC(CC(C3)C1)C2]

### Synthesis of 287.1_1 and 287.1_2

Thionyl chloride (3 mL) was added to 110.4_1a (300 mg, 0.65 mmol) at RT. After stirring for 2 h, the reaction mixture was concentrated under reduced pressure to afford intermediate acid chloride. To the above prepared acid chloride in CH₂Cl₂ (5 mL) was added solution of (3s,5s,7s)-N-methyladamantan-1-amine (161 mg, 0.97 mmol) in CH₂Cl₂ (5 mL). After stirring for 16 h at 50° C., the reaction was quenched with water (10 mL) and extracted with CH₂Cl₂ (2×15 mL). The combined organic layer was washed with brine, dried over anhydrous Na₂SO₄, filtered and concentrated under reduced pressure. The residue was purified by flash column chromatography (40 g Silica gel cartridge, 20%-25% EtOAc/pet ether) to obtain 287.1_1 (Peak-1, 105 mg, 26%) as a pale brown solid and 287.1_2 (Peak-2, 84 mg, 21%) as a pale brown solid.

287.1_1: LCMS: 24.6%, m/z [M+H]⁺=608.1.

287.1_2: LCMS: 81.9%, m/z [M+H]⁺=608.1.

### Synthesis of 287

To a stirred solution of 287.1_1 (105 mg, 0.17 mmol) in THF (5 mL) were added aniline (16 mg, 0.17 mmol) and Pd(PPh₃)₄ (39 mg, 0.03 mmol) at RT. After stirring for 2 h, the reaction mixture was concentrated under reduced pressure. The residue was purified by prep. HPLC [X-BRIDGE-C18 (150×30) mm, 5 μ; A: 0.1% Formic acid in H₂O, B: Acetonitrile; Gradient: (Time/%B): 0/65, 8/80, 8.1/98, 10/98, 10.1/98, 13/65 at 22 mL/min] to obtain 287 (16 mg, 15%) as solid.

¹H NMR (500 MHz, DMSO-d₆): 12.40 (br s, 1H), 11.06 (br s, 1H), 7.68 (s, 1H), 7.52 (s, 1H), 4.13 (d, J=11.0 Hz, 1H), 3.89-3.85 (m, 1H), 3.72-3.67 (m, 1H), 3.06 (s, 3H), 2.86-2.64 (m, 3H), 2.50-2.47 (m, 1H), 2.13-2.04 (m, 9H), 1.65-1.60 (m, 6H); LCMS: 94.1%, m/z [M+H]⁺=568.1; Chiral purity: 90.5%.

### Example 288

### Synthesis of 1′-((2-(3-(tert-butyl)phenyl)-2-methylpropyl)carbamoyl)-5,7-dichloro-6′,6′-difluoro-2-oxo-1′,2′,5′,6′,7′,7a′-hexahydrospiro[indoline-3,3′-pyrrolizine]-2′-carboxylic acid (288a) & rac-(1′R,2′S,3R,7a′R)-1′-((2-(3-(tert-butyl)phenyl)-2-methylpropyl)carbamoyl)-5,7-dichloro-6′,6′-difluoro-2-oxo-1′,2′,5′,6′,7′,7a′-hexahydrospiro[indoline-3,3′-pyrrolizine]-2′-carboxylic acid (288b)

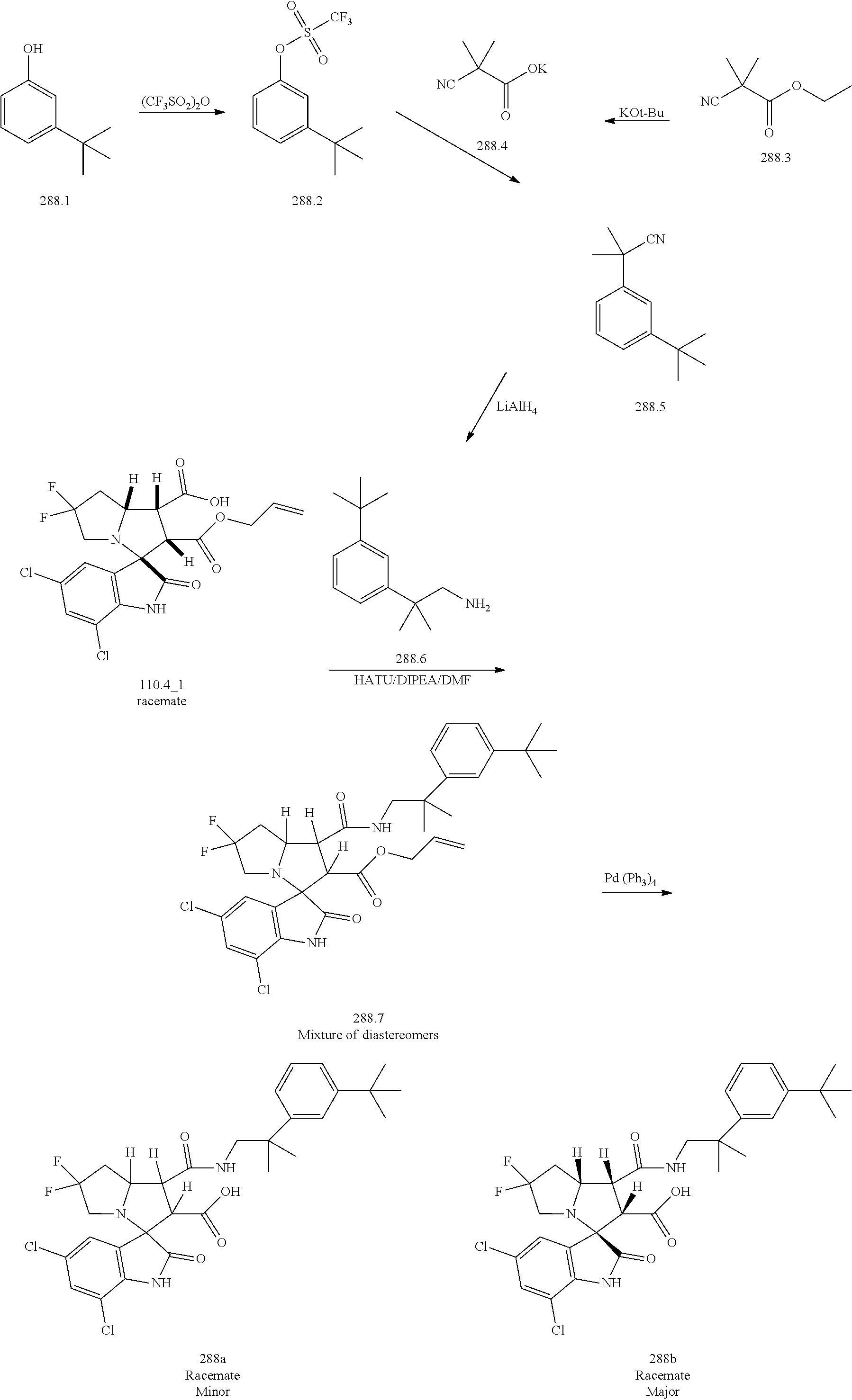

### Synthesis of 288.2

To a stirred solution of 288.1 (5 g, 33.2 mmol) in CH₂Cl₂ (60 mL) were added pyridine (2.9 mL, 36.5 mmol) and trifluoromethanesulfonic anhydride (6.1 mL, 36.5 mmol) at 0° C. After stirring for 16 h at RT, the reaction mixture was quenched with ice water. The organic layer was separated and washed with water (60 mL), brine (60 mL), dried over Na₂SO₄, and concentrated under reduced pressure. The residue was purified by flash column chromatography (40 g Silica gel cartridge, 10% EtOAc in pet ether) to afford 288.2 (3.9 g, 42%) as a colorless oil.

¹H NMR (500 MHz, CDCl₃): 7.42-7.35 (m, 2H), 7.26-7.25 (m, 1H), 7.10-7.07 (m, 1H), 1.33 (s, 9H).

### Synthesis of 288.4

To a stirred solution of 288.3 (3 g, 21.2 mmol) in EtOH (40 mL) and water (401 mg, 22.2 mmol) at RT was added a solution of potassium tert-butoxide (2.4 g, 21.2 mmol) in EtOH (20 mL) at 60° C. over a period of 30 minutes. After stirring for 2 h at 60° C., the reaction mixture was concentrated under reduced pressure. The residue was triturated with a mixture of diethyl ether and EtOH to afford 288.4 (1.8 g, 56%) as a solid.

¹H NMR (500 MHz, DMSO-d₆): 1.29 (s, 6H).

### Synthesis of 288.5

A stirred suspension of 288.2 (2 g, 7.08 mmol), 288.4 (1.28 g, 8.5 mmol) and Xantphos (205 mg, 0.35 mmol) in mesitylene (20 mL) was purged with argon for 15 minutes followed by addition of bis(allyl)dichlorodipalladium (78 mg, 0.21 mmol) and purging with argon for an additional 5 minutes. After stirring at 130° C. for 6 h, the reaction mixture was cooled to RT and poured into ice water (30 mL). The organic layer was separated, washed with water (20 mL), brine (20 mL), dried over Na₂SO₄, filtered and concentrated under reduced pressure. The residue was purified by flash column chromatography (80 g Silica gel cartridge, 10% EtOAc in pet ether to afford 288.5 (1.2 g, 72%) as a colorless oil.

¹H NMR (500 MHz, CDCl₃): 7.51 (d, J=1.5 Hz, 1H), 7.36-7.25 (m, 3H), 1.73 (s, 6H), 1.34 (s, 9H); GCMS: 99.4%, m/z [M+H]⁺=202.2.

### Synthesis of 288.6

To a stirred solution of 288.5 (1 g, 4.96 mmol) in THF (15 mL) was added LiAlH₄ solution (1M in THF, 7.5 mL, 7.45 mmol) at 0° C. After stirring for 4 h at 0° C., the reaction mixture was quenched with 10% NaOH solution at 0° C. and extracted with EtOAc (20 mL). The organic layer was separated, washed with water (20 mL), brine (20 mL), dried over Na₂SO₄, filtered and concentrated under reduced pressure. The residue was triturated with n-pentane (10 mL) to afford 288.6 (850 mg, 84%) as a solid.

¹H NMR (500 MHz, DMSO-d₆): 7.34 (d, J=2.0 Hz, 1H), 7.34-7.13 (m, 3H), 3.62-3.59 (m, 1H), 2.67 (s, 2H), 1.78-1.76 (m, 1H), 1.28 (s, 9H), 1.23 (s, 6H); LCMS: 92.9%, m/z [M+H]⁺=206.2.

### Synthesis of 288.7

To a stirred solution of 110.4_1 (300 mg, 0.65 mmol) in DMF (3 mL) were added DIPEA (252 mg, 1.95 mmol) and HATU (297 mg, 0.77 mmol) at RT. After stirring for 15 minutes at RT, 288.6 (200 mg, 0.97 mmol) was added. After stirring for 6 h at the RT, the reaction mixture was quenched with water (10 mL) and extracted with EtOAc (10 mL). The combined organic layer was washed with water (20 mL), brine (20 mL), dried over anhydrous Na₂SO₄, filtered and concentrated under reduced pressure to afford mixture of diastereomers of 288.7 (500 mg) as a brown gummy material. The residue was used in the next step without purification.

### Synthesis of 288a & 288b

To a stirred solution of 288.7 (500 mg, 0.77 mmol) in THF (20 mL) were added aniline (70 mg, 0.77 mmol) and Pd(PPh₃)₄ (180 mg, 0.2 mmol) at RT. After stirring for 2 h at RT, the reaction mixture was concentrated under reduced pressure and triturated with diethyl ether and pentane. The residue was purified by prep. HPLC [X-BRIDGE C18 (150×30) mm, 5 μ; A: 0.1% Formic acid in H₂O, B: Acetonitrile; Gradient: (Time/%B): 0/65, 9/80, 11/80, 11.1/65, 14/65 at 25 mL/min] to afford minor diastereomer 288a (20 mg, 5%) as a solid and major diastereomer 288b (57 mg, 14%) as a solid.

288a: ¹H NMR (500 MHz, DMSO-d₆): 12.50 (br s, 1H), 11.07 (br s, 1H), 7.54-7.39 (m, 2H), 7.31 (s, 1H), 7.24-7.17 (m, 3H), 7.10-7.07 (m, 1H), 4.15-4.05 (m, 2H), 3.57-3.47 (m, 2H), 3.32-3.20 (m, 2H), 2.76-2.64 (m, 2H), 2.31-2.24 (m, 1H), 1.39-1.30 (m, 9H), 1.28-1.26 (m, 6H); LCMS: 98.5%, m/z [M+H]⁺=608.3. (absolute stereochemistry not determined)

288b: ¹H NMR (500 MHz, DMSO-d₆): 12.35 (br s, 1H), 10.93 (br s, 1H), 8.19 (br s, 1H), 7.88 (br s, 1H), 7.42 (d, J=1.5 Hz, 2H), 7.23-7.19 (m, 3H), 3.92-3.86 (m, 2H), 3.63-3.59 (m, 1H), 3.47-3.40 (m, 1H), 3.16-3.01 (m, 2H), 2.56-2.50 (m, 1H), 1.94-1.87 (m, 1H), 1.71-1.58 (m, 1H), 1.33-1.26 (m, 15H); LCMS: 98.5%, m/z [M+H]⁺=608.3.

TABLE 8

| | | | M/Z | M/Z | |
|---|---|---|---|---|---|
| Example | aniline | Compound | (M + H)+ | (M − H)− | 1H NMR |
| 289 | 3,5-dichloro-N- ethylaniline | 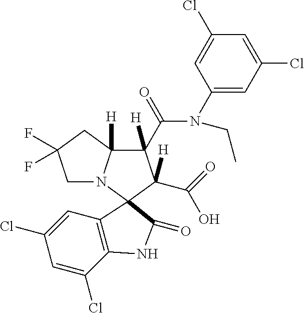 | | 590.2 | ¹H NMR (500 MHz, DMSO-d₆) (Exist in rolameric form): 12.55 (br s, 1H), 11.05/10.99 (br s, 1H), 8.30/7.93 (s, 1H), 7.69-7.37 (m, 4H), 4.01-4.00 (m, 1H), 3.92- 3.89 (m, 1H), 3.84-3.79 (m, 1H), 3.57-3.53 (m, 1H), 3.45-3.44 (m, 1H), 3.32-3.26 (m, 1H), 2.60- 2.50 (m, 1H), 2.50-2.36 (m, 1H), 2.19-2.03 (m, 1H), 1.10-1.04 (m, 3H). |
| 290 | 3,5-dichloro-4- (difluoro- methoxy)-N- methylaniline | 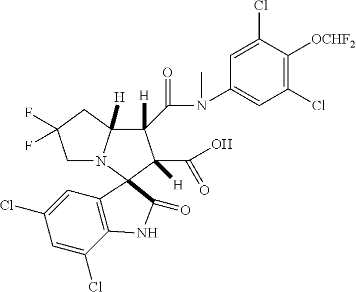 | 644.0 | | ¹H NMR (500 MHz, DMSO-d₆) (Exist in rotameric form): 12.65 (br s, 1H), 11.05/10.99 (s, 1H), 8.67-7.01 (m, 4H), 4.17-4.12 (m, 1H), 4.01- 3.99 (m, 1H), 3.85-3.81 (m, 1H), 3.55 (t, J = 6.5 Hz, 1H), 3.40/3.24 (s, 3H), 3.29-3.10 (m, 1H), 2.67-2.54 (m, 1H), 2.39- 2.35 (m, 1H), 2.15-2.02 (m, 1H). |
| 291 | 3,5-dichloro-4- methoxy-N- methylaniline | 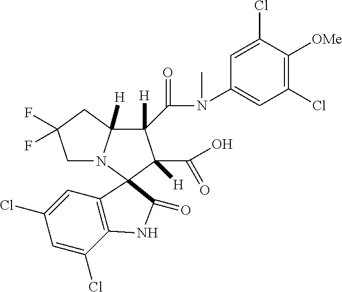 | 608.0 | | ¹H NMR (500 MHz, DMSO-d₆) (Exist in rotameric form): 12.60 (br s, 1H), 11.05/10.97 (s, 1H), 8.31/7.89 (s, 1H), 7.64-7.45 (m, 3H), 4.15- 3.97 (m, 1H), 3.86 (s, 3H), 3.86-3.80 (m, 1H), 3.58-3.50 (m, 1H), 3.37/3.17 (s, 3H), 3.33- 3.20 (m, 1H), 2.60-2.50 (m, 1H), 2.42-2.30 (m, 1H), 2.18-2.05 (m, 1H). |
| 292 | N-methyl-6- (trifluoromethyl) pyridin-2- amine | 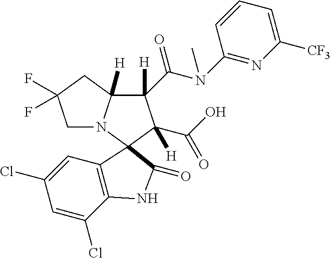 | 579.2 | | ¹H NMR (500 MHz, DMSO-d₆) (Exist in rotameric form): 12.38 (br s, 1H), 11.03 (br s, 1H), 8.21 (br s, 1H), 7.82-7.47 (m, 4H), 4.07- 3.92 (m, 1H), 3.82-3.72 (m, 1H), 3.42/3.32 (br s, 3H), 3.32-3.25 (m, 1H), 3.16-3.08 (m, 1H), 2.70- 2.60 (m, 1H), 2.50-2.40 (m, 1H), 2.17-2.00 (m, 1H). |
| 293 | 2-(3-(2- aminoethyl) phenyl)-2- methylpropan- 1-ol | 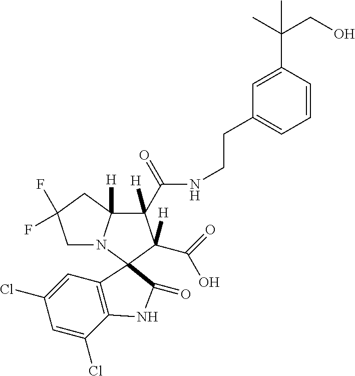 | 596.3 | | ¹H NMR (500 MHz, DMSO-d₆): 12.60 (br s, 1H), 11.10 (br s, 1H), 8.05 (br s, 1H), 7.60-7.50 (m, 1H), 7.22-7.17 (m, 4H), 7.03-7.02 (m, 1H), 4.70-4.60 (m, 1H), 3.90- 3.70 (m, 1H), 3.45-3.34 (m, 3H), 3.32-3.28 (m, 2H), 2.92-2.80 (m, 1H), 2.76-2.63 (m, 3H), 2.36- 2.23 (m, 2H), 1.20 (s, 6H). |
| 294 | (1-(3-(tert- butyl)phenyl) cyclopropyl) methanamine | 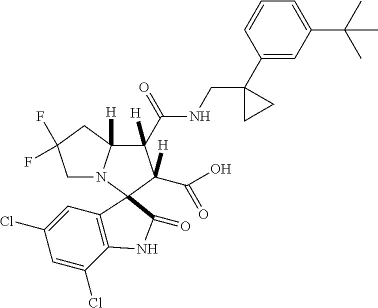 | 606 | | ¹H NMR (500 MHz, DMSO-d₆): 12.27 (br s, 1H), 10.96 (br s, 1H), 8.22 (br s, 1H), 8.18-8.09 (m, 1H), 7.46-7.43 (m, 1H), 7.34 (s, 1H), 7.22- 7.14 (m, 3H), 3.92-3.86 (m, 2H), 3.83-3.78 (m, 1H), 3.42-3.39 (m, 1H), 3.09-3.04 (m, 2H), 2.54- 2.50 (m, 1H), 1.97-1.86 (m, 1H), 1.75-1.60 (m, 1H), 1.27 (s, 9H), 0.95- 0.84 (m, 3H), 0.71-0.68 (m, 1H). |

Using 110.4_1 and the listed aniline, the following compounds were made as in Example 288.

Assays for Detecting and Measuring the Effect of Compounds on dF508-CFTR Channels CFRT-YFP High Throughput Assay:

The following protocol is designed to selectively screen small molecule compounds for F508del CFTR corrector activities in the HTS YFP flux assay. In this protocol, the cells are incubated with test compounds for 24 hours, washed with PBS, stimulated with forskolin and a standard potentiator, and read on a 384-well HTS plate reader, such as the Hamamatsu FDDD-6000.

YFP fluorescence intensity is acquired at high speed before and after iodide buffer is injected to the assay cells. Iodide enters the cells via active CFTR channels in the plasma membrane, and quenches the YFP fluorescence. The rate of fluorescence quenching is proportionally related to the total CFTR activities in the cell membrane. dF508-CFTR corrector accelerates YFP quenching by increasing the number of CFTR molecules in the testing cell plasma membrane.

This method was initially developed for bench top plate readers (Galietta et al., 2001), and was adapted to the HTS format (Sui et al. Assay Drug Dev. Technol. 2010).

Fisher Rat Thyroid (FRT) cells stably expressing both human ΔF508-CFTR and a halide-sensitive yellow fluorescent protein (YFP-H148Q/I152L 25, 22) (Galietta et al., Am.J.Physiol Cell Physiol 281(5), C_{1734, 2001}) were cultured on plastic surface in Coon's modified Ham's F12 medium supplemented with FBS 10%, L-glutamine 2 mM, penicillin 100 U/mL, and streptomycin 100 μg/mL. G418 (0.75-1.0 mg/mL) and zeocin (3.2 ug/mL) were used for selection of FRT cells expressing ΔF508-CFTR and YFP. For primary screening, FRT cells were plated into 384-well black wall, transparent bottom microtiter plates (Costar; Corning Inc.) at a cell density of 20,000-40,000 per well. Test compounds were applied to the cells at varying concentrations. Cells were incubated in a cell culture incubator at 37° C. with 5% CO₂ for 24-26 hr. Assay plates were washed with DPBS media (Thermo, cat# SH30028.02) to remove unbound cells and compound. Stimulation media (25 μL) containing 20 μM Forskolin & 30 μM P3 [6-(Ethyl-phenyl-sulfonyl)-4-oxo-1, 4-dihydro-quinoline-3-carboxylic acid 2-methoxy-benzylamide] in Hams F-12 Coon's modified media was added to the plate wells and incubated at RT for 60-120 min. 25 μL of HEPES-PBS-I buffer (10 mM HEPES, 1 mM MgCl₂, 3 mM KCl, 1 mM CaCl₂, 150 mM NaI) was then added and fluorescence quench curves (Excitation 500 nm/Emission 540 nm; exposure 136 ms) were immediately recorded on an FDSS-6000 plate reader (Hamamatsu). Quench rates were derived from least squares fitting of the data as described by Sui et al., (2010).

### REFERENCES

Galietta, L. J., Jayaraman, S., and Verkman, A. S. Cell-based assay for high-throughput quantitative screening of CFTR chloride transport agonists. Am.J.Physiol Cell Physiol 281(5), C_{1734, 2001.}

Sui J, Cotard S, Andersen J, Zhu P, Staunton J, Lee M, Lin S. (2010) Optimization of a Yellow fluorescent protein-based iodide influx high-throughput screening assay for cystic fibrosis transmembrane conductance regulator (CFTR) modulators. Assay Drug Dev Technol. 2010 Dec; 8(6):656-68.

Alejandro A. Pezzulo, Xiao Xiao Tang, Mark J. Hoegger, Mahmoud H. Abou Alaiwa, Shyam Ramachandran, Thomas O. Moninger, Phillip H. Karp, Christine L. Wohlford-Lenane, Henk P. Haagsman, Martin van Eijk, Botond Ba'nfi, Alexander R. Horswill, David A. Stoltz, Paul B. McCray Jr, Michael J. Welsh, Joseph Zabner. Reduced airway surface pH impairs bacterial killing in the porcine cystic fibrosis lung. Nature 487, 109-115 (2012).

### Determination of Activity in Primary CF Cell:

### Cell Culture:

Primary CF airway epithelial cells were obtained from the UNC Cystic Fibrosis Tissue Procurement and Cell Culture Core. The cells are grown at 37° C. in a Heracell 150i incubator using growth media (BEGM, Fischer). Cells were then transferred to differentiation media (airway liquid interface media (ALI) media; Lechner JF and LaVeck MA, J. Tissue Culture Methods 1985, 9: 43-48) for a minimum of 4 weeks on coated Costar snapwells. Two days before the Ussing assay the mucus on the apical surface of the cells was aspirated after incubating with 200 μL of differentiation media for at least thirty (30) minutes. One day before the Ussing assay test compounds were added to the basolateral surface of the cells at various test concentrations dissolved in DMSO. Duplicate wells were prepared giving a n=3 or n=4 protocol.

### Electrophysiological Procedures

Cells were treated for 24 hours with various combinations and concentrations of the test articles, reference standard (3 μM VX809 or 3 μM FDL169, positive control). Compounds stock solutions were prepared in DMSO and diluted 1/1000 into ALI media to their final assay concentration. Cells were treated with combination solutions (2 mL of each dilution) and incubated at 37° C. for 24 h.

### Ussing Assay:

For an Ussing assay, cells on four Snapwell (6-well) plates were treated 24 hours prior to experimentation. The next day filters from individual Snapwells were removed from the plates and mounted vertically in Ussing chambers pre-equilibrated at 37° C. in 5 ml of HBS (pH 7.4) both apical and basolateral sides and bubbled with room air to facilitate mixing upon addition of compounds. The resting current was recorded for 10 min to ensure a stable baseline. Resting current was blocked by the apical addition of 3 μM benzamil, an ENaC inhibitor. After 10 min, 10 μM forskolin was added to both the apical and basolateral side to stimulate CFTR. The increase in chloride current was detected as an upward deflection of the trace. After an additional 10 min, the potentiator VX₇₇₀ (1 μM) was added, further increasing the chloride current. Finally CFTR-172 (a CFTR inhibitor, 20 μM) and/or bumetanide (20 μM) was added to block CFTR mediated chloride current, resulting in a decrease in the observed current.

### Equivalent Current Assay

For the equivalent current assay, cells on four Transwell (24-well) plates were treated. Each Transwell plate was filled with 200 μl of HBS on the apical surface and 2 ml on the basolateral surface. Plates were placed horizontally in a heated mount at 37° C., and equilibrated for several minutes. Resting current was measured for 15 min and then blocked by the apical addition of 5 μM benzamil. After 20 min, 10 μM forskolin and 1 μM VX₇₇₀ were added to both the apical and basolateral side to stimulate CFTR. An increase in chloride current is seen as an upward deflection of the trace. After another 30 min, CFTR-172 (a CFTR inhibitor, 20 μM) and/or bumetanide (20 uM) was added to block CFTR mediated chloride current.

### Data Collection and Analysis Methods

The raw data, current vs. time for the Ussing chamber (sampling interval: 10 s) and voltage vs. time and resistance vs. time for the equivalent current assay (sampling interval: 5 minutes) were transferred to Excel (Microsoft Office Professional, version 14.0.7106.5003) for analysis. CFTR specific current was measured as the average amplitude of the increase in current elicited upon addition forskolin and ending upon addition of the CFTR channel specific blocker CFTR-172. This average is equivalent to the sum of the average forskolin activated and the average VX770-potentiated currents. The average current measured in vehicle (0.1% DMSO) treated cells, Iv, was subtracted from the current for the test article, ITA, or from the corrector reference standard VX₈₀₉ (3 uM ISTD). For replicate measurements, the average vehicle subtracted response for the test article, was normalized to the average vehicle subtracted inhibitor response of the reference corrector VX₈₀₉ (3 μM).

I_{NSC}=(I_{TA}−I_{V})₍ₐᵥₑ₎/(I_{STD}−I_{V})₍ₐᵥₑ₎ (Equation 1)

A second endpoint, for the equivalent current assay, evaluated was NAUC, the normalized area under the curve (AUC) measuring the response after addition of forskolin and VX770 to the time point right before the addition of the CFTR inhibitor. The AUC is effectively the average response multiplied by the duration of the response. The AUC of the test article, AUC_{TA} was then corrected by subtracting the average vehicle response, AUC_{V,ave} over the same time range, and normalized as for the inhibitor-sensitive current to the difference of the corrector reference standard VX809 (3 μM _{VX809r,ave}) or FDL176 (3 μM _{FDL176r,ave}) and the vehicle response:

NAUC_{TA}=[AUC_{TA}−AUC_{V,ave}]/[AUC_{Vx809r,ave}−AUC_{V,ave}] (Equation 2).

The normalized value for DMSO is 0.0 and for VX-809 alone is 1.0. Combinations of compounds with VX-809 that give normalized values greater than 1.0 show activity in the combination assay. A value of 2 means the test compound doubles the effect to VX-809 alone.

Experiments were run with a minimum of n=4 replicates per concentration. Since the distribution for the ratio of two normal distributions is a Cauchy distribution, the median value must be used for the average and the average deviation must be used for the error of all normalized data. Potency (EC₅₀) and efficacy (maximum response) were determined by fitting dose response data to a sigmoid dose response model (GraphPad Prism 5.04, Manufacturer) using Equation 3:

E=Eₘᵢₙ+(Eₘₐₓ−Eₘᵢₙ)/(1+10\hat{ }((LogEC5OS)*n_{H})) (Equation 3)

where E is the recorded response, and S is the concentration of test compound in combination with VX-809. Since there were at most 8 points in the dose response curve only EC₅₀ and maximum (Eₘₐₓ) were allowed to vary, while the minimum (Eₘᵢₙ) was fixed equal to the VX-809 response of 1.0, and the Hill slope, n_{H}, was fixed equal to 1.

Statistical comparisons (t-test and Mann Whitney) and calculation of averages and errors were performed in Excel.

The table below provides the results of the equivalent current assays in Primary CF airway epithelial for cells treated with combination solutions (2 mL of each dilution: either FDL169 (3 μM) or VX-809 (3 μM) as noted in column 5-1st site corrector) and incubated at 37° C. for 24 h.

NAUC “+++” refers to an observed NAUC>170% of positive control; NAUC “++” refers to an observed NAUC 170-140% of positive control; NAUC “+” refers to an observed NAUC <140% of positive control.

| | Nauc @ | Nauc @ | Nauc @ | 1^{st} site |
|---|---|---|---|---|
| Example | 1 μM | 3 μM | 10 μM | corrector |
| 1 | | +++ | +++ | VX-809 |
| 2 | | + | ++ | VX-809 |
| 3 | | +++ | +++ | VX-809 |
| 4 | | +++ | +++ | VX-809 |
| 5 | | +++ | +++ | VX-809 |
| 6 | | +++ | +++ | VX-809 |
| 7 | | | +++ | VX-809 |
| 8 | | +++ | +++ | VX-809 |
| 9 | | +++ | +++ | VX-809 |
| 10 | | ++ | ++ | VX-809 |
| 11 | | +++ | ++ | VX-809 |
| 12 | | +++ | +++ | VX-809 |
| 13 | | +++ | +++ | VX-809 |
| 14 | | +++ | +++ | VX-809 |
| 15 | | +++ | +++ | VX-809 |
| 16 | | +++ | +++ | VX-809 |
| 17 | | +++ | +++ | VX-809 |
| 18 | | + | +++ | VX-809 |
| 19 | | ++ | +++ | VX-809 |
| 20 | | +++ | +++ | VX-809 |
| 21 | | +++ | +++ | VX-809 |
| 22 | | ++ | +++ | VX-809 |
| 23 | | ++ | + | VX-809 |
| 24 | | +++ | + | VX-809 |
| 25 | | + | ++ | VX-809 |
| 26 | | ++ | +++ | VX-809 |
| 27 | | ++ | ++ | VX-809 |
| 28 | | +++ | +++ | VX-809 |
| 29 | | ++ | +++ | VX-809 |
| 30 | | + | ++ | VX-809 |
| 31 | | + | +++ | VX-809 |
| 32 | | +++ | +++ | VX-809 |
| 33 | | +++ | +++ | VX-809 |
| 34 | | +++ | ++ | VX-809 |
| 35 | | +++ | +++ | VX-809 |
| 36 | | + | +++ | VX-809 |
| 37 | | +++ | +++ | VX-809 |
| 38 | | + | ++ | VX-809 |
| 39 | | +++ | +++ | VX-809 |
| 40 | | +++ | +++ | VX-809 |
| 41 | | + | ++ | VX-809 |
| 42 | | +++ | +++ | VX-809 |
| 43 | | +++ | ++ | VX-809 |
| 44 | | +++ | +++ | VX-809 |
| 45 | | +++ | +++ | VX-809 |
| 46 | | +++ | +++ | VX-809 |
| 47 | | ++ | +++ | VX-809 |
| 48 | | ++ | +++ | VX-809 |
| 49 | | ++ | +++ | VX-809 |
| 50 | | ++ | +++ | VX-809 |
| 51 | | +++ | +++ | VX-809 |
| 52 | | ++ | +++ | VX-809 |
| 53 | | +++ | +++ | VX-809 |
| 54 | | + | +++ | VX-809 |
| 55 | | ++ | + | VX-809 |
| 56 | | +++ | +++ | VX-809 |
| 57 | | +++ | +++ | VX-809 |
| 58 | | + | +++ | VX-809 |
| 59 | | + | ++ | VX-809 |
| 60 | | +++ | +++ | VX-809 |
| 61 | | +++ | +++ | VX-809 |
| 62 | | | +++ | VX-809 |
| 63 | | | +++ | VX-809 |
| 64 | | | +++ | VX-809 |
| 65 | | | +++ | VX-809 |
| 66.9a | | | + | VX-809 |
| 66.9b.1 | | | + | VX-809 |
| 66.9b.2 | | | + | VX-809 |
| 66.9b.3 | | | + | VX-809 |
| 67.9a | | | + | VX-809 |
| 67.9b | + | ++ | +++ | VX-809 |
| 68.a | | | + | VX-809 |
| 68.b | | | + | VX-809 |
| 68.c | + | +++ | +++ | VX-809 |
| 69.a | | | + | VX-809 |
| 69.b | ++ | +++ | ++ | VX-809 |
| 70.a | | | + | VX-809 |
| 70.b | | | + | VX-809 |
| 70.c | + | + | +++ | VX-809 |
| 71.a | | | +++ | VX-809 |
| 72.a | | | + | VX-809 |
| 72.b | | | + | VX-809 |
| 72.c | | | ++ | VX-809 |
| 73.a | | | + | VX-809 |
| 73.b | | | + | VX-809 |
| 73.c | + | + | +++ | VX-809 |
| 74.a | | | + | VX-809 |
| 75.a | | | + | VX-809 |
| 76.a | | | + | VX-809 |
| 76.b | | | + | VX-809 |
| 76.c | | | + | VX-809 |
| 76.d | | | + | VX-809 |
| 77.a | | | + | VX-809 |
| 77.b | | | + | VX-809 |
| 78.a | | | + | VX-809 |
| 79a | + | + | + | VX-809 |
| 79b | + | + | ++ | VX-809 |
| 79c | + | + | + | VX-809 |
| 80.7a.1 | | | + | VX-809 |
| 80.7a.2 | + | +++ | +++ | VX-809 |
| 80.7b.1 | | | + | VX-809 |
| 80.7b.2 | | | + | VX-809 |
| 81 | + | ++ | + | VX-809 |
| 82.6a | +++ | ++ | | FDL169 |
| 82.6b | ++ | + | | FDL169 |
| 83b | +++ | +++ | | FDL169 |
| 84b | +++ | + | | FDL169 |
| 90 | + | + | | FDL169 |
| 91 | ++ | ++ | | FDL169 |
| 92 | ++ | +++ | +++ | VX-809 |
| 92 | + | ++ | +++ | FDL169 |
| 93 | +++ | +++ | ++ | FDL169 |
| 94 | ++ | ++ | | FDL169 |
| 95 | +++ | +++ | +++ | FDL169 |
| 96 | +++ | +++ | + | FDL169 |
| 100 | + | + | + | VX-809 |
| 101 | + | + | | FDL169 |
| 102 | | | + | VX-809 |
| 103 | + | + | + | FDL169 |
| 104 | | | + | FDL169 |
| 110 | +++ | +++ | + | FDL169 |
| 111 | + | ++ | | FDL169 |
| 112 | +++ | +++ | +++ | VX-809 |
| 113 | + | + | + | VX-809 |
| 114 | +++ | ++ | +++ | VX-809 |
| 115 | + | + | + | VX-809 |
| 116 | +++ | +++ | ++ | VX-809 |
| 117 | + | ++ | + | VX-809 |
| 118 | + | + | + | VX-809 |
| 119 | + | + | + | VX-809 |
| 120 | + | + | + | VX-809 |
| 121 | +++ | +++ | ++ | VX-809 |
| 122 | + | + | | VX-809 |
| 123 | + | + | | VX-809 |
| 124 | +++ | +++ | | VX-809 |
| 125 | + | + | | VX-809 |
| 126 | ++ | ++ | | VX-809 |
| 127 | + | + | | VX-809 |
| 128 | + | + | | VX-809 |
| 129 | + | + | | VX-809 |
| 130 | + | + | | VX-809 |
| 131 | +++ | +++ | | VX-809 |
| 132 | +++ | +++ | +++ | VX-809 |
| 133 | +++ | +++ | +++ | VX-809 |
| 134 | + | + | | VX-809 |
| 135 | + | + | + | VX-809 |
| 136 | + | + | | FDL169 |
| 137 | + | + | | FDL169 |
| 138 | + | + | | FDL169 |
| 139 | + | + | | FDL169 |
| 140 | + | + | | FDL169 |
| 141 | + | + | | FDL169 |
| 142 | + | + | | FDL169 |
| 143 | + | + | | FDL169 |
| 144 | + | + | | FDL169 |
| 145 | +++ | +++ | +++ | FDL169 |
| 146 | + | + | | FDL169 |
| 147 | + | + | | FDL169 |
| 148 | +++ | +++ | +++ | FDL169 |
| 149 | ++ | +++ | + | FDL169 |
| 150 | + | +++ | | FDL169 |
| 151 | +++ | +++ | ++ | FDL169 |
| 152 | +++ | +++ | ++ | FDL169 |
| 153 | + | + | | FDL169 |
| 154 | +++ | +++ | + | FDL169 |
| 155 | +++ | +++ | + | FDL169 |
| 156 | +++ | +++ | ++ | FDL169 |
| 157 | ++ | ++ | | FDL169 |
| 158 | + | + | | FDL169 |
| 159 | + | + | | FDL169 |
| 160 | + | + | | FDL169 |
| 161 | ++ | +++ | ++ | FDL169 |
| 162 | +++ | +++ | +++ | FDL169 |
| 163 | +++ | +++ | ++ | FDL169 |
| 164 | +++ | +++ | +++ | FDL169 |
| 165 | + | + | | FDL169 |
| 166 | + | + | | FDL169 |
| 167 | + | + | | FDL169 |
| 168 | + | + | | FDL169 |
| 169 | ++ | +++ | +++ | FDL169 |
| 170 | +++ | + | + | VX-809 |
| 171 | +++ | +++ | +++ | FDL169 |
| 172 | +++ | +++ | +++ | FDL169 |
| 173 | +++ | +++ | + | FDL169 |
| 174 | +++ | +++ | +++ | FDL169 |
| 175 | +++ | +++ | +++ | FDL169 |
| 176 | +++ | +++ | | FDL169 |
| 177 | +++ | ++ | ++ | FDL169 |
| 178 | +++ | +++ | ++ | FDL169 |
| 179 | ++ | ++ | +++ | FDL169 |
| 180 | +++ | +++ | + | FDL169 |
| 181 | ++ | +++ | | FDL169 |
| 182 | +++ | ++ | | FDL169 |
| 185 | +++ | +++ | + | FDL169 |
| 188 | +++ | ++ | ++ | FDL169 |
| 189 | ++ | + | | FDL169 |
| 191 | ++ | + | | FDL169 |
| 192 | ++ | + | | FDL169 |
| 193 | ++ | + | | FDL169 |
| 194 | ++ | +++ | | FDL169 |
| 200 | ++ | ++ | ++ | FDL169 |
| 200 | +++ | +++ | +++ | VX-809 |
| 201 | + | + | | FDL169 |
| 202 | ++ | ++ | + | FDL169 |
| 203 | + | + | | FDL169 |
| 205 | + | ++ | ++ | FDL169 |
| 207 | ++ | ++ | | FDL169 |
| 208 | ++ | ++ | + | FDL169 |
| 219 | ++ | + | | FDL169 |
| 227 | + | ++ | ++ | VX-809 |
| 228 | + | + | + | VX-809 |
| 230 | | | + | VX-809 |
| 231 | | | + | VX-809 |
| 233 | ++ | +++ | +++ | VX-809 |
| 236 | + | ++ | +++ | VX-809 |
| 237 | + | + | ++ | VX-809 |
| 238 | + | + | | VX-809 |
| 239 | + | + | + | VX-809 |
| 240 | + | + | + | VX-809 |
| 241 | ++ | +++ | ++ | VX-809 |
| 242 | + | + | + | VX-809 |
| 243 | + | + | | VX-809 |
| 244 | + | + | + | VX-809 |
| 245 | + | ++ | +++ | VX-809 |
| 246 | + | + | +++ | VX-809 |
| 247 | + | ++ | +++ | VX-809 |
| 248 | + | +++ | +++ | VX-809 |
| 249 | +++ | +++ | +++ | VX-809 |
| 257 | | | +++ | VX-809 |
| 261 | | | + | VX-809 |
| 266 | + | +++ | +++ | VX-809 |
| 267 | +++ | +++ | +++ | VX-809 |
| 271 | + | +++ | + | VX-809 |
| 272 | + | + | ++ | VX-809 |
| 282 | + | + | | FDL169 |
| 283 | + | + | | FDL169 |
| 289 | +++ | +++ | +++ | VX-809 |
| 290 | ++ | +++ | +++ | VX-809 |
| 291 | + | + | +++ | VX-809 |
| 292 | ++ | +++ | +++ | VX-809 |
| 293 | | | + | VX-809 |
| 294 | + | +++ | +++ | VX-809 |
| 225a | + | + | +++ | VX-809 |
| 225b | + | +++ | +++ | VX-809 |
| 226a | ++ | ++ | +++ | VX-809 |
| 226b | + | +++ | +++ | VX-809 |
| 229b | | | + | VX-809 |
| 232a | + | + | + | VX-809 |
| 232b | + | + | + | VX-809 |
| 234a | ++ | ++ | ++ | VX-809 |
| 234b | + | ++ | +++ | VX-809 |
| 235a | ++ | +++ | +++ | VX-809 |
| 235b | +++ | +++ | +++ | VX-809 |
| 250.3a | | | +++ | VX-809 |
| 250.3b | | | + | VX-809 |
| 251a | | | + | VX-809 |
| 251b | | | + | VX-809 |
| 251c | | | + | VX-809 |
| 256a | | | +++ | VX-809 |
| 256b | | | + | VX-809 |
| 260a | + | + | | VX-809 |
| 260b | + | + | + | VX-809 |
| 265a | + | +++ | +++ | VX-809 |
| 265b | + | + | + | VX-809 |
| 268a | + | + | + | VX-809 |
| 268b | +++ | +++ | | VX-809 |
| 269a | + | + | + | VX-809 |
| 269b | ++ | +++ | +++ | FDL169 |
| 269b | +++ | +++ | +++ | VX-809 |
| 270a | +++ | +++ | ++ | FDL169 |
| 270b | + | ++ | | FDL169 |
| 273a | + | + | | FDL169 |
| 273b | + | ++ | | FDL169 |
| 274.6a | ++ | + | + | FDL169 |
| 274.6b | +++ | ++ | + | FDL169 |
| 275.7a | +++ | +++ | +++ | FDL169 |
| 275.7b | +++ | +++ | ++ | FDL169 |
| 288a | +++ | +++ | +++ | VX-809 |
| 288b | | | + | VX-809 |

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

1. A compound of Formula (I) 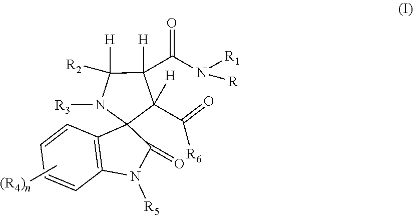
or a pharmaceutically acceptable salt thereof, wherein:
R and R₁ are independently selected from hydrogen, optionally substituted alkyl; optionally substituted alkenyl, optionally substituted alkynyl, optionally substituted cycloalkyl, optionally substituted aryl, optionally substituted arylalky, optionally substituted heteroaryl or optionally substituted heteroarylalkyl;
or R and R₁, together with the nitrogen atom to which they are attached, form an optionally substituted 3 to 7-membered heterocyclyl;
R₂ is hydrogen, optionally substituted alkyl, optionally substituted aryl, optionally substituted arylalkyl, optionally substituted heteroaryl or optionally substituted heteroarylalkyl;
R₃ is hydrogen, optionally substituted alkyl, R₇C(O)—, R₇SO₂— or R₇NHC(O)—;
or R₂ and R₃, together with the atoms to which they are attached, form an optionally substituted 3 to 7-membered heterocyclyl;
Each R₄ is independently halogen, optionally substituted alkyl, CN, optionally substituted alkoxy, NR₁₂R₁₃, or hydroxy;
R₅ is hydrogen, optionally substituted alkyl, optionally substituted alkenyl, optionally substituted alkynyl, or optionally substituted cycloalkyl;
R₆ is OR₈, SR₈ or NR₉R₁₀;
R₇ is optionally substituted alkyl, optionally substituted cycloalkyl, optionally substituted aryl, or optionally substituted arylalkyl;
R₈ is hydrogen, optionally substituted alkyl; optionally substituted alkenyl, optionally substituted alkynyl, optionally substituted cycloalkyl, optionally substituted aryl or optionally substituted heteroaryl;
R₉ is hydrogen, OR₁₁, optionally substituted alkyl, optionally substituted alkenyl, optionally substituted alkynyl, optionally substituted cycloalkyl; optionally substituted aryl, optionally substituted heteroaryl, heterocyclyl, SO₂—R₈, SO₂NRₐR_{b} or N(Rₐ)R_{b};
R₁₀ is hydrogen; optionally substituted alkyl, optionally substituted alkenyl, optionally substituted alkynyl, optionally substituted cycloalkyl; optionally substituted aryl or optionally substituted heteroaryl;
or R₉ and R₁₀, together with the nitrogen atom to which they are attached, form an optionally substituted heterocyclyl;
Rₐ and R_{b} are each independently hydrogen, optionally substituted alkyl; optionally substituted alkenyl, optionally substituted alkynyl, optionally substituted cycloalkyl, optionally substituted aryl or optionally substituted heteroaryl;
R₁₁ is hydrogen or optionally substituted alkyl;
R₁₂ and R₁₃ are each independently hydrogen, optionally substituted alkyl, R₇C(O)—, R₇SO₂— or R₇NHC(O)—;
or R₁₂ and R₁₃, together with the nitrogen atom to which they are attached, form an optionally substituted heterocyclyl;
and
n is 0, 1, 2, 3 or 4.

2. The compound of claim 1, wherein R₁ is optionally substituted aryl, optionally substituted heteroaryl, optionally substituted arylalkyl or optionally substituted heteroarylalkyl; preferably optionally substituted phenyl or optionally substituted 6-membered heteroaryl.

3. The compound of claim 1, wherein R is hydrogen, optionally substituted C₁-C₆-alkyl; optionally substituted C₃-C₈-cycloalkyl.

4. (canceled)

5. The compound of claim 1, wherein R₂ is hydrogen, optionally substituted C₁-C₆-alkyl, optionally substituted aryl-C₁-C₆-alkyl, or optionally substituted heteroaryl-C₁-C₆-alkyl.

6.-8 (canceled)

9. The compound of claim 1, wherein R₃ is hydrogen, C₁-C₄-alkyl, halo-C₁-C₄-alkyl, C₁-C₄-alkylC(O)—, aryl-C₁-C₄-alkylS(O)₂—, aryl-C₁-C₄-alkylNHC(O)—, or arylNHC(O)—.

10.-13. (canceled)

14. The compound of claim 1, represented by Formula II, 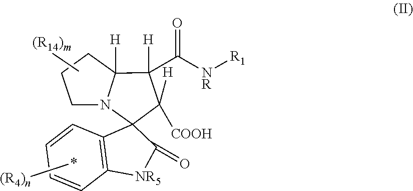
or a pharmaceutically acceptable salt thereof, wherein
m is 0, 1, 2 or 3; and
each R₁₄ is independently hydroxyl, protected hydroxyl, cyano, amino, protected amino, halogen, optionally substituted alkoxy, optionally substituted alkyl, optionally substituted alkylSO₂—, optionally substituted alkylC(O)—, or optionally substituted alkylC(O)NH—; or
two adjacent R₁₄ groups together with the carbon atoms to which they are attached form an optionally substituted fused 3 to 7-membered carbocylyl or heterocyclyl; or
two geminal R₁₄ groups together with the carbon atom to which they are attached form an optionally substituted spiro 3 to 7-membered carbocyclyl or heterocyclyl; or
two geminal R₁₄ groups together form (R₁₃)₂C=, wherein each R₁₃ is independently halogen, C₁-C₄-alkyl or halo-C₁-C₄-alkyl.

15. The compound of claim 1, represented by Formula III, 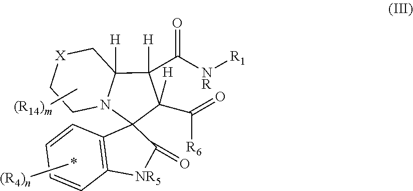
wherein X is O or C(Rₐ)₂;
each R₁₄ is independently hydroxyl, protected hydroxyl, cyano, amino, protected amino, halogen, optionally substituted alkoxy, optionally substituted alkyl, optionally substituted alkylSO₂—, optionally substituted alkylC(O)—, or optionally substituted alkylC(O)NH—; or
two adjacent R₁₄ groups together with the carbon atoms to which they are attached form an optionally substituted fused 3 to 7-membered carbocylyl or heterocyclyl; or
two geminal R₁₄ groups together with the carbon atom to which they are attached form an optionally substituted spiro 3 to 7-membered carbocyclyl or heterocyclyl; or
two geminal R₁₄ groups together form (R₁₃)₂C=, wherein each R₁₃ is independently halogen, C₁-C₄-alkyl or halo-C₁-C₄-alkyl; and
each Rₐ is independently hydrogen, hydroxyl, protected hydroxyl, cyano, amino, protected amino, halogen, optionally substituted alkoxy, or optionally substituted alkyl.

16.-18. (canceled)

19. The compound of claim 15, wherein

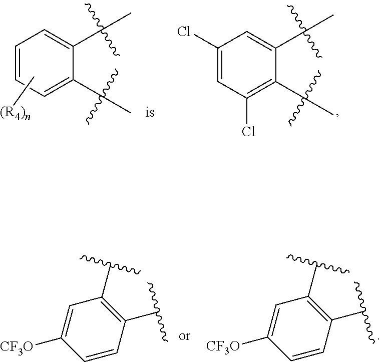

20. The compound of claim 19, wherein

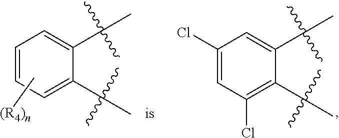

21. The compound of claim 14, represented by Formula IV, 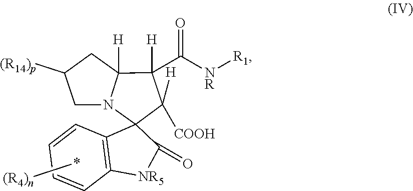
or a pharmaceutically acceptable salt thereof, wherein p is 0, 1 or 2.

22. The compound of claim 21, wherein p is 1 or 2, and each R₁₄ is independently selected from the group consisting of amino, protected amino, cyano, hydroxyl, fluoro, chloro, C₁-C₄-alkoxy, halo-C₁-C₄-alkoxy, C₁-C₄-alkyl SO₂, C₁-C₄-alkylC(O), C₁-C₄-alkylC(O)NH— and C₁-C₄-alkyl optionally substituted with one or more substituents independently selected from the group consisting of hydroxyl, fluoro, chloro, and amino; or
p is 2 and the two R₁₄ groups together with the carbon atom to which they are attached form a spiro C₃-C₆-cycloalkyl or a spiro-3 to 6-membered heterocycloalkyl; or
p is 2 and the two R₁₄ groups together form (R₁₃)₂C=, wherein each R₁₃ is independently hydrogen, fluoro, chloro, methyl, CF₃ or CHF₂.

23.-29. (canceled)

30. The compound of claim 21, wherein R₁ is selected from the groups set forth below:

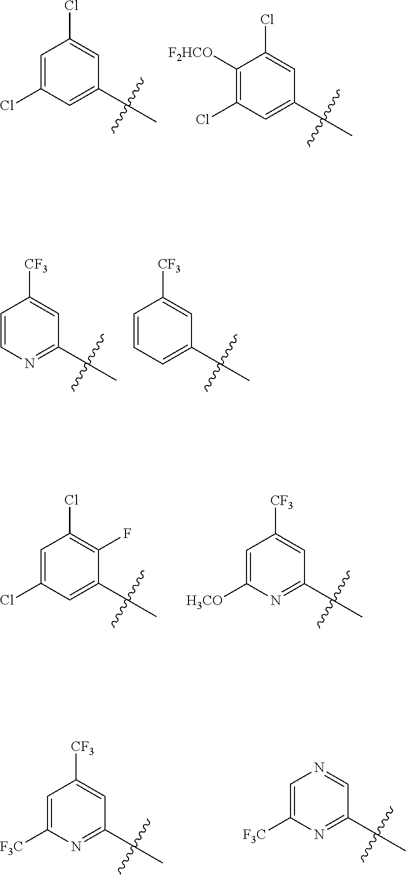

31. The compound of claim 21, wherein R₁ is represented by 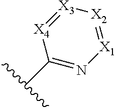
where X₁-X₄ are each independently N or CR₁₇, wherein R₁₇ is hydrogen, optionally substituted alkyl, optionally substituted alkoxy or halogen; or 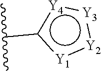
wherein one of Y₁, Y₂, Y₃ and Y₄ is O, S or NR₁₆, and the remainder are independently N or CR₁₇, wherein R₁₆ is hydrogen, optionally substituted alkyl, R₇C(O)—, R₇SO₂— or R₇NHC(O); R₁₇ is hydrogen, optionally substituted alkyl, optionally substituted alkoxy or halogen; and
R₇ is optionally substituted alkyl, optionally substituted cycloalkyl, optionally substituted aryl, or optionally substituted arylalkyl.

32. The compound of claim 31, wherein R₁ is selected from the groups shown below:

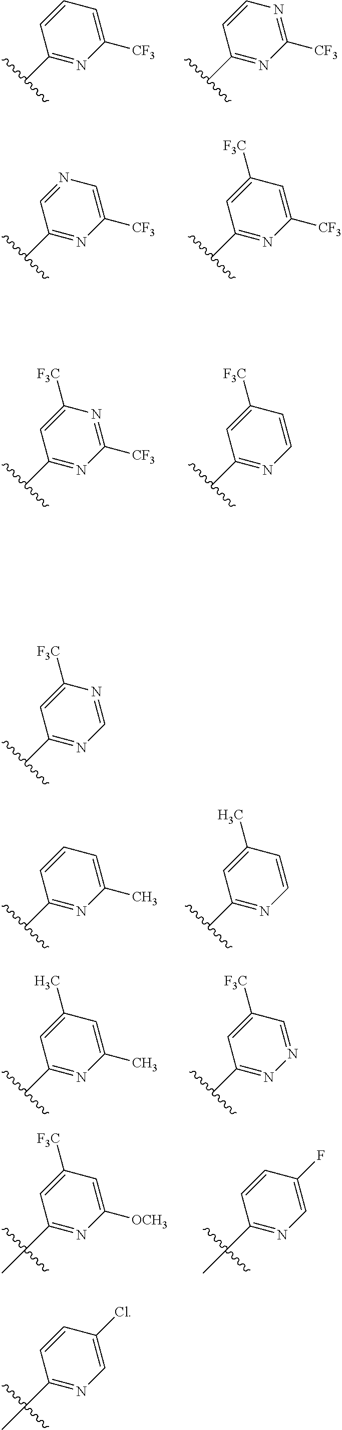

33. (canceled)

34. (anceled)

35. The compound of claim 31, wherein R₁ is selected from the groups shown below:

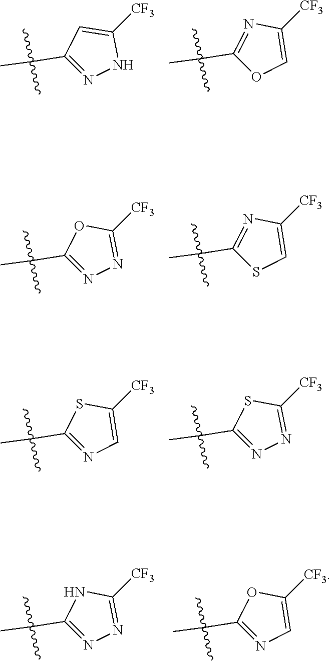

36. A compound selected from the compounds set forth in the table below, or a pharmaceutically acceptable salt thereof,

| Compound No. | Structure |
|---|---|
| 1 | 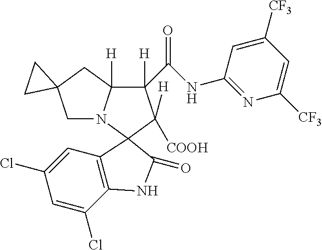 [SMILES: O=C(Nc1cc(C(F)(F)F)cc(C(F)(F)F)n1)C1C2CC3(CC3)CN2C2(C(=O)Nc3c(Cl)cc(Cl)cc32)C1C(=O)O] |
| 2 | 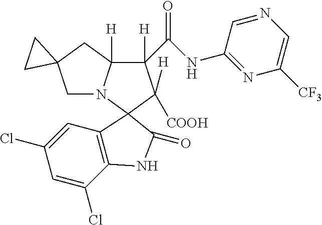 [SMILES: O=C(Nc1cncc(C(F)(F)F)n1)C1C2CC3(CC3)CN2C2(C(=O)Nc3c(Cl)cc(Cl)cc32)C1C(=O)O] |
| 3 | 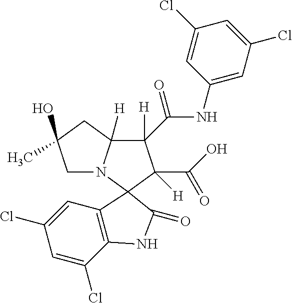 [SMILES: C[C@]1(O)CC2C(C(=O)Nc3cc(Cl)cc(Cl)c3)C(C(=O)O)C3(C(=O)Nc4c(Cl)cc(Cl)cc43)N2C1] |
| 4 | 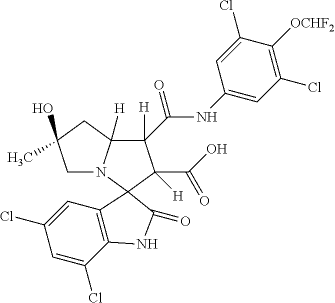 [SMILES: C[C@]1(O)CC2C(C(=O)Nc3cc(Cl)c(OC(F)F)c(Cl)c3)C(C(=O)O)C3(C(=O)Nc4c(Cl)cc(Cl)cc43)N2C1] |
| 5 | 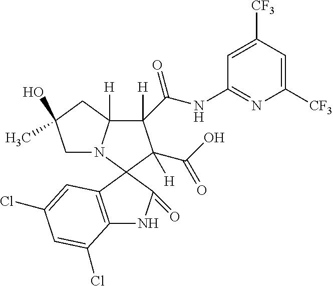 [SMILES: C[C@]1(O)CC2C(C(=O)Nc3cc(C(F)(F)F)cc(C(F)(F)F)n3)C(C(=O)O)C3(C(=O)Nc4c(Cl)cc(Cl)cc43)N2C1] |
| 6 | 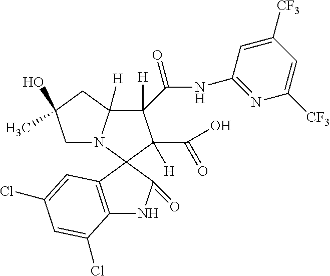 [SMILES: C[C@]1(O)CC2C(C(=O)Nc3cc(C(F)(F)F)cc(C(F)(F)F)n3)C(C(=O)O)C3(C(=O)Nc4c(Cl)cc(Cl)cc43)N2C1] |
| 7 | 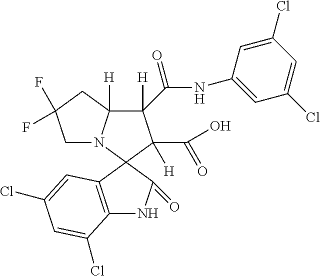 [SMILES: O=C(Nc1cc(Cl)cc(Cl)c1)C1C2CC(F)(F)CN2C2(C(=O)Nc3c(Cl)cc(Cl)cc32)C1C(=O)O] |
| 8 | 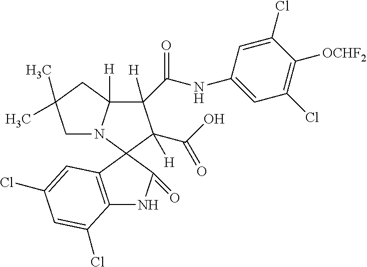 [SMILES: CC1(C)CC2C(C(=O)Nc3cc(Cl)c(OC(F)F)c(Cl)c3)C(C(=O)O)C3(C(=O)Nc4c(Cl)cc(Cl)cc43)N2C1] |
| 9 | 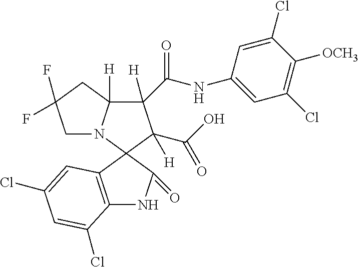 [SMILES: COc1c(Cl)cc(NC(=O)C2C3CC(F)(F)CN3C3(C(=O)Nc4c(Cl)cc(Cl)cc43)C2C(=O)O)cc1Cl] |
| 10 | 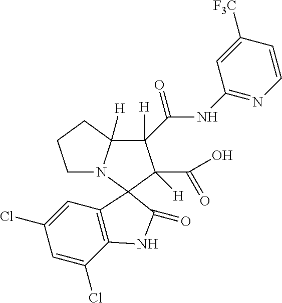 [SMILES: O=C(Nc1cc(C(F)(F)F)ccn1)C1C2CCCN2C2(C(=O)Nc3c(Cl)cc(Cl)cc32)C1C(=O)O] |
| 11 | 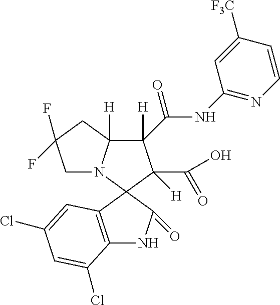 [SMILES: O=C(Nc1cc(C(F)(F)F)ccn1)C1C2CC(F)(F)CN2C2(C(=O)Nc3c(Cl)cc(Cl)cc32)C1C(=O)O] |
| 12 | 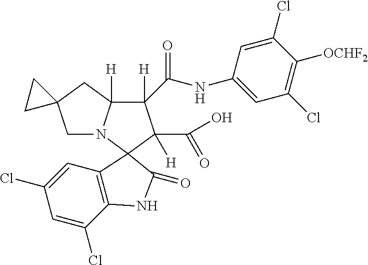 [SMILES: O=C(Nc1cc(Cl)c(OC(F)F)c(Cl)c1)C1C2CC3(CC3)CN2C2(C(=O)Nc3c(Cl)cc(Cl)cc32)C1C(=O)O] |
| 13 | 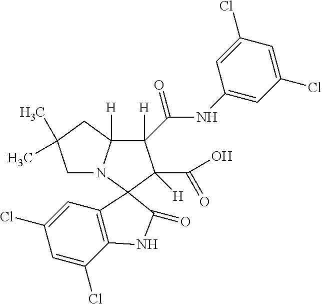 [SMILES: CC1(C)CC2C(C(=O)Nc3cc(Cl)cc(Cl)c3)C(C(=O)O)C3(C(=O)Nc4c(Cl)cc(Cl)cc43)N2C1] |
| 14 | 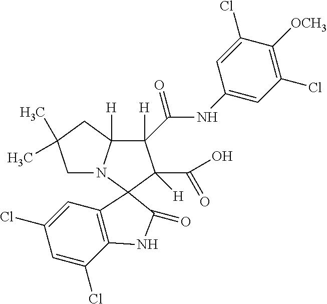 [SMILES: COc1c(Cl)cc(NC(=O)C2C3CC(C)(C)CN3C3(C(=O)Nc4c(Cl)cc(Cl)cc43)C2C(=O)O)cc1Cl] |
| 15 | 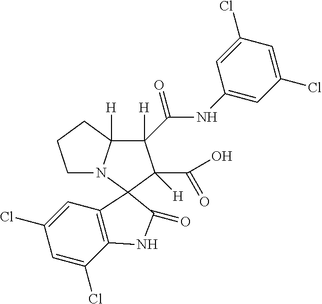 [SMILES: O=C(Nc1cc(Cl)cc(Cl)c1)C1C2CCCN2C2(C(=O)Nc3c(Cl)cc(Cl)cc32)C1C(=O)O] |
| 16 | 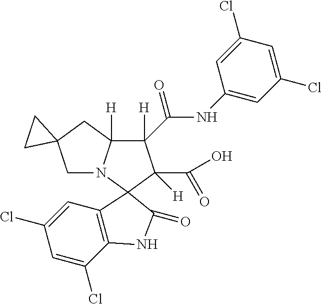 [SMILES: O=C(Nc1cc(Cl)cc(Cl)c1)C1C2CC3(CC3)CN2C2(C(=O)Nc3c(Cl)cc(Cl)cc32)C1C(=O)O] |
| 17 | 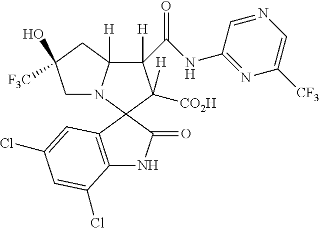 [SMILES: O=C(Nc1cncc(C(F)(F)F)n1)C1C2C[C@@](O)(C(F)(F)F)CN2C2(C(=O)Nc3c(Cl)cc(Cl)cc32)C1C(=O)O] |
| 18 | 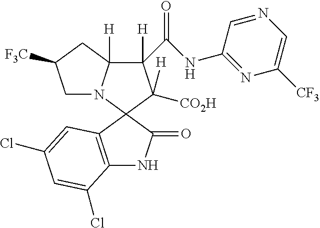 [SMILES: O=C(Nc1cncc(C(F)(F)F)n1)C1C2C[C@H](C(F)(F)F)CN2C2(C(=O)Nc3c(Cl)cc(Cl)cc32)C1C(=O)O] |
| 19 | 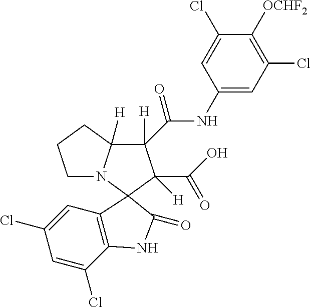 [SMILES: O=C(Nc1cc(Cl)c(OC(F)F)c(Cl)c1)C1C2CCCN2C2(C(=O)Nc3c(Cl)cc(Cl)cc32)C1C(=O)O] |
| 20 | 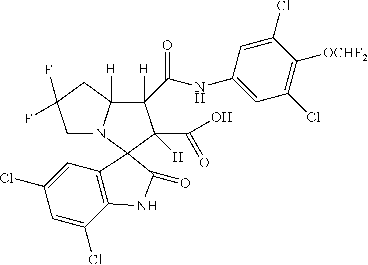 [SMILES: O=C(Nc1cc(Cl)c(OC(F)F)c(Cl)c1)C1C2CC(F)(F)CN2C2(C(=O)Nc3c(Cl)cc(Cl)cc32)C1C(=O)O] |
| 21 | 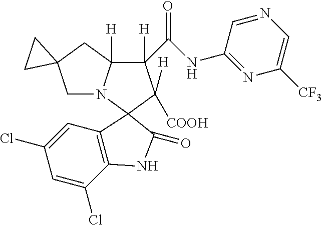 [SMILES: O=C(Nc1cncc(C(F)(F)F)n1)C1C2CC3(CC3)CN2C2(C(=O)Nc3c(Cl)cc(Cl)cc32)C1C(=O)O] |
| 22 | 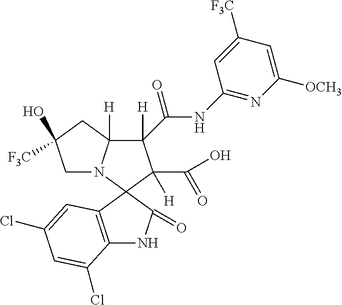 [SMILES: COc1cc(C(F)(F)F)cc(NC(=O)C2C3C[C@@](O)(C(F)(F)F)CN3C3(C(=O)Nc4c(Cl)cc(Cl)cc43)C2C(=O)O)n1] |
| 23 | 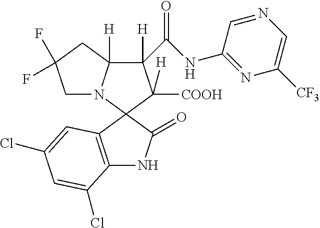 [SMILES: O=C(Nc1cncc(C(F)(F)F)n1)C1C2CC(F)(F)CN2C2(C(=O)Nc3c(Cl)cc(Cl)cc32)C1C(=O)O] |
| 24 | 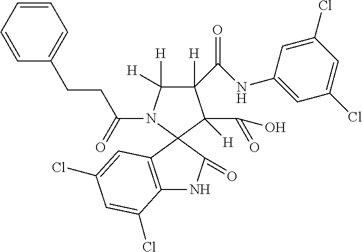 [SMILES: O=C(Nc1cc(Cl)cc(Cl)c1)C1CN(C(=O)CCc2ccccc2)C2(C(=O)Nc3c(Cl)cc(Cl)cc32)C1C(=O)O] |
| 25 | 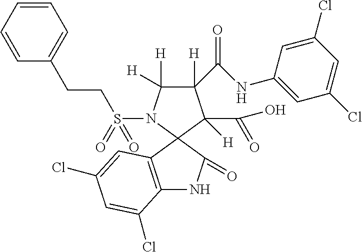 [SMILES: O=C(Nc1cc(Cl)cc(Cl)c1)C1CN(S(=O)(=O)CCc2ccccc2)C2(C(=O)Nc3c(Cl)cc(Cl)cc32)C1C(=O)O] |
| 26 | 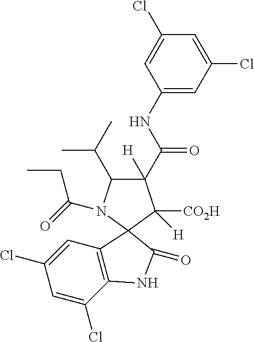 [SMILES: CCC(=O)N1C(C(C)C)C(C(=O)Nc2cc(Cl)cc(Cl)c2)C(C(=O)O)C12C(=O)Nc1c(Cl)cc(Cl)cc12] |
| 27 | 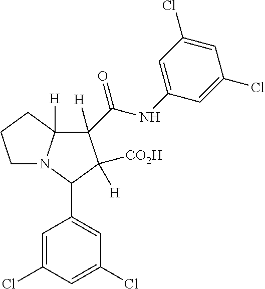 [SMILES: O=C(Nc1cc(Cl)cc(Cl)c1)C1C(C(=O)O)C(c2cc(Cl)cc(Cl)c2)N2CCCC12] |
| 28 | 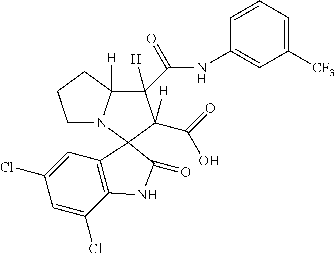 [SMILES: O=C(Nc1cccc(C(F)(F)F)c1)C1C2CCCN2C2(C(=O)Nc3c(Cl)cc(Cl)cc32)C1C(=O)O] |
| 29 | 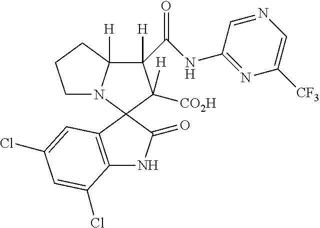 [SMILES: O=C(Nc1cncc(C(F)(F)F)n1)C1C2CCCN2C2(C(=O)Nc3c(Cl)cc(Cl)cc32)C1C(=O)O] |
| 30 | 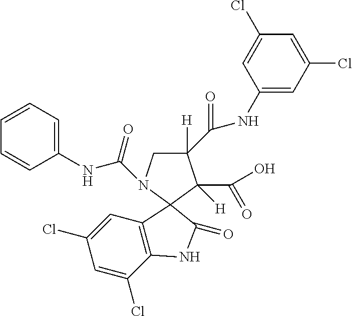 [SMILES: O=C(Nc1cc(Cl)cc(Cl)c1)C1CN(C(=O)Nc2ccccc2)C2(C(=O)Nc3c(Cl)cc(Cl)cc32)C1C(=O)O] |
| 31 | 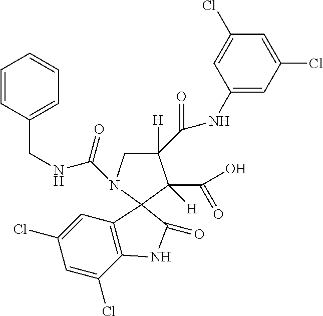 [SMILES: O=C(Nc1cc(Cl)cc(Cl)c1)C1CN(C(=O)NCc2ccccc2)C2(C(=O)Nc3c(Cl)cc(Cl)cc32)C1C(=O)O] |
| 32 | 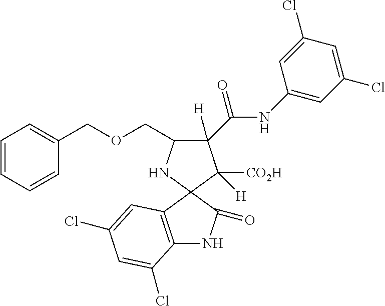 [SMILES: O=C(Nc1cc(Cl)cc(Cl)c1)C1C(COCc2ccccc2)NC2(C(=O)Nc3c(Cl)cc(Cl)cc32)C1C(=O)O] |
| 33 | 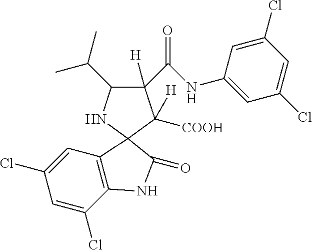 [SMILES: CC(C)C1NC2(C(=O)Nc3c(Cl)cc(Cl)cc32)C(C(=O)O)C1C(=O)Nc1cc(Cl)cc(Cl)c1] |
| 34 | 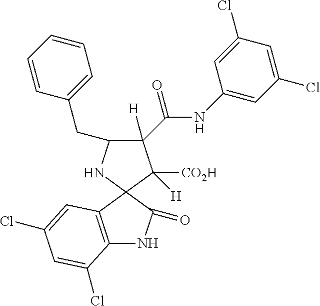 [SMILES: O=C(Nc1cc(Cl)cc(Cl)c1)C1C(Cc2ccccc2)NC2(C(=O)Nc3c(Cl)cc(Cl)cc32)C1C(=O)O] |
| 35 | 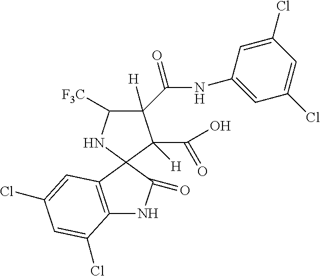 [SMILES: O=C(Nc1cc(Cl)cc(Cl)c1)C1C(C(F)(F)F)NC2(C(=O)Nc3c(Cl)cc(Cl)cc32)C1C(=O)O] |
| 36 | 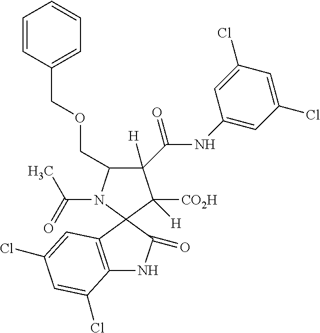 [SMILES: CC(=O)N1C(COCc2ccccc2)C(C(=O)Nc2cc(Cl)cc(Cl)c2)C(C(=O)O)C12C(=O)Nc1c(Cl)cc(Cl)cc12] |
| 37 | 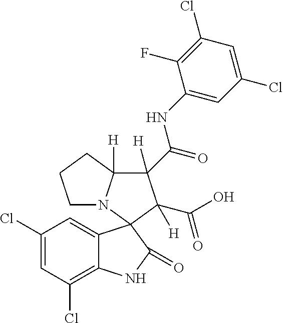 [SMILES: O=C(Nc1cc(Cl)cc(Cl)c1F)C1C2CCCN2C2(C(=O)Nc3c(Cl)cc(Cl)cc32)C1C(=O)O] |
| 38 | 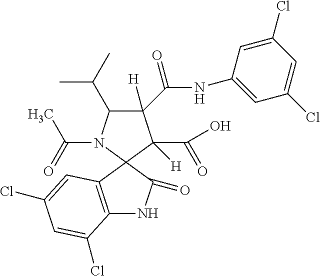 [SMILES: CC(=O)N1C(C(C)C)C(C(=O)Nc2cc(Cl)cc(Cl)c2)C(C(=O)O)C12C(=O)Nc1c(Cl)cc(Cl)cc12] |
| 39 | 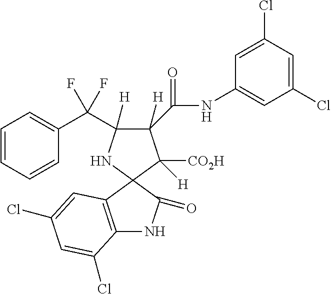 [SMILES: O=C(Nc1cc(Cl)cc(Cl)c1)C1C(C(F)(F)c2ccccc2)NC2(C(=O)Nc3c(Cl)cc(Cl)cc32)C1C(=O)O] |
| 40 | 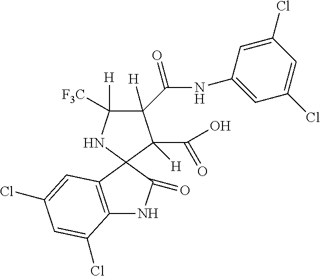 [SMILES: O=C(Nc1cc(Cl)cc(Cl)c1)C1C(C(F)(F)F)NC2(C(=O)Nc3c(Cl)cc(Cl)cc32)C1C(=O)O] |
| 41 | 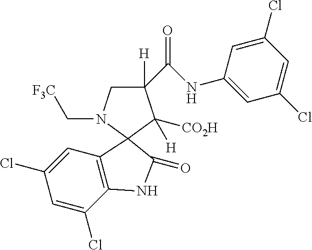 [SMILES: O=C(Nc1cc(Cl)cc(Cl)c1)C1CN(CC(F)(F)F)C2(C(=O)Nc3c(Cl)cc(Cl)cc32)C1C(=O)O] |
| 42 | 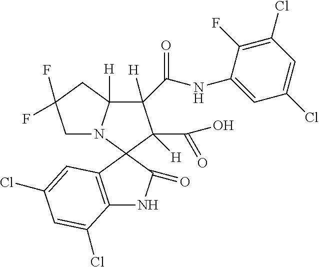 [SMILES: O=C(Nc1cc(Cl)cc(Cl)c1F)C1C2CC(F)(F)CN2C2(C(=O)Nc3c(Cl)cc(Cl)cc32)C1C(=O)O] |
| 43 | 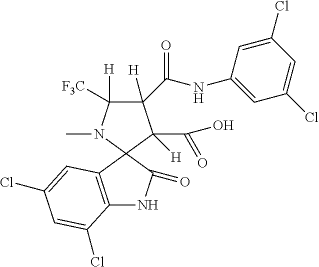 [SMILES: CN1C(C(F)(F)F)C(C(=O)Nc2cc(Cl)cc(Cl)c2)C(C(=O)O)C12C(=O)Nc1c(Cl)cc(Cl)cc12] |
| 44 | 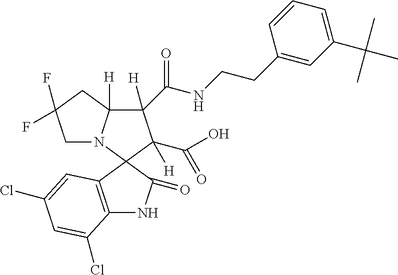 [SMILES: CC(C)(C)c1cccc(CCNC(=O)C2C3CC(F)(F)CN3C3(C(=O)Nc4c(Cl)cc(Cl)cc43)C2C(=O)O)c1] |
| 45 | 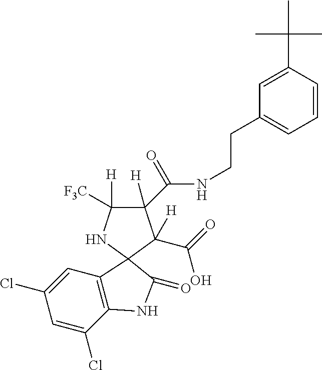 [SMILES: CC(C)(C)c1cccc(CCNC(=O)C2C(C(F)(F)F)NC3(C(=O)Nc4c(Cl)cc(Cl)cc43)C2C(=O)O)c1] |
| 46 | 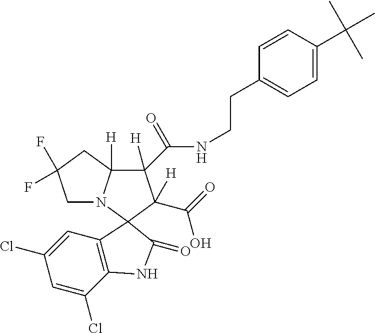 [SMILES: CC(C)(C)c1ccc(CCNC(=O)C2C3CC(F)(F)CN3C3(C(=O)Nc4c(Cl)cc(Cl)cc43)C2C(=O)O)cc1] |
| 47 | 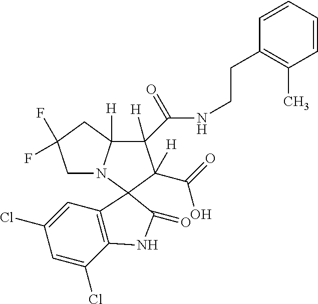 [SMILES: Cc1ccccc1CCNC(=O)C1C2CC(F)(F)CN2C2(C(=O)Nc3c(Cl)cc(Cl)cc32)C1C(=O)O] |
| 48 | 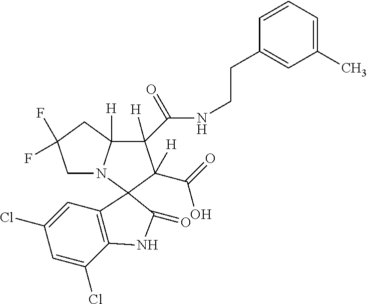 [SMILES: Cc1cccc(CCNC(=O)C2C3CC(F)(F)CN3C3(C(=O)Nc4c(Cl)cc(Cl)cc43)C2C(=O)O)c1] |
| 49 | 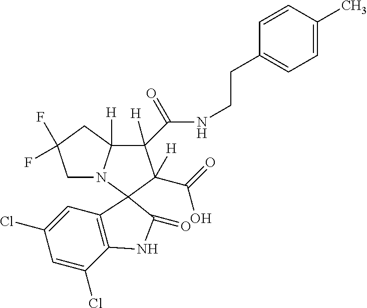 [SMILES: Cc1ccc(CCNC(=O)C2C3CC(F)(F)CN3C3(C(=O)Nc4c(Cl)cc(Cl)cc43)C2C(=O)O)cc1] |
| 50 | 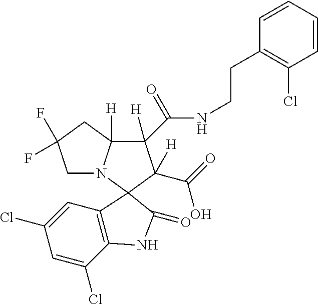 [SMILES: O=C(NCCc1ccccc1Cl)C1C2CC(F)(F)CN2C2(C(=O)Nc3c(Cl)cc(Cl)cc32)C1C(=O)O] |
| 51 | 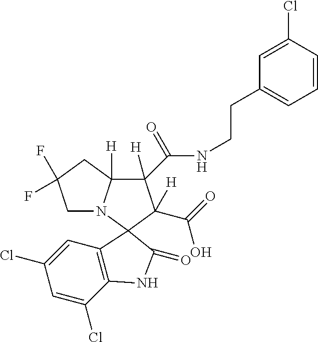 [SMILES: O=C(NCCc1cccc(Cl)c1)C1C2CC(F)(F)CN2C2(C(=O)Nc3c(Cl)cc(Cl)cc32)C1C(=O)O] |
| 52 | 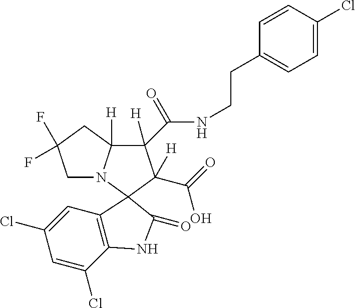 [SMILES: O=C(NCCc1ccc(Cl)cc1)C1C2CC(F)(F)CN2C2(C(=O)Nc3c(Cl)cc(Cl)cc32)C1C(=O)O] |
| 53 | 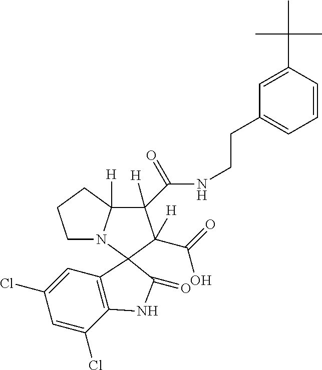 [SMILES: CC(C)(C)c1cccc(CCNC(=O)C2C3CCCN3C3(C(=O)Nc4c(Cl)cc(Cl)cc43)C2C(=O)O)c1] |
| 54 | 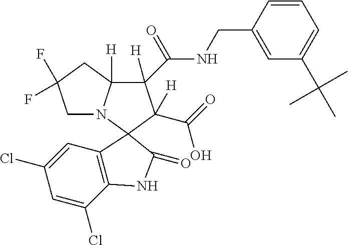 [SMILES: CC(C)(C)c1cccc(CNC(=O)C2C3CC(F)(F)CN3C3(C(=O)Nc4c(Cl)cc(Cl)cc43)C2C(=O)O)c1] |
| 55 | 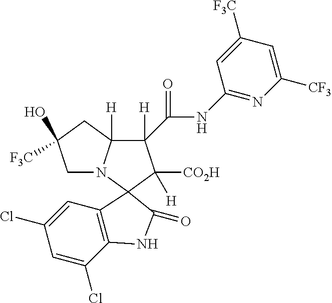 [SMILES: O=C(Nc1cc(C(F)(F)F)cc(C(F)(F)F)n1)C1C2C[C@@](O)(C(F)(F)F)CN2C2(C(=O)Nc3c(Cl)cc(Cl)cc32)C1C(=O)O] |
| 56 | 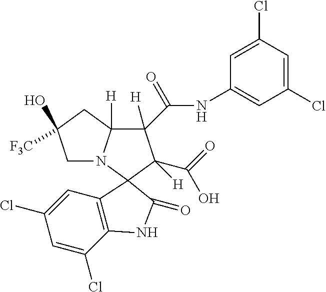 [SMILES: O=C(Nc1cc(Cl)cc(Cl)c1)C1C2C[C@@](O)(C(F)(F)F)CN2C2(C(=O)Nc3c(Cl)cc(Cl)cc32)C1C(=O)O] |
| 57 | 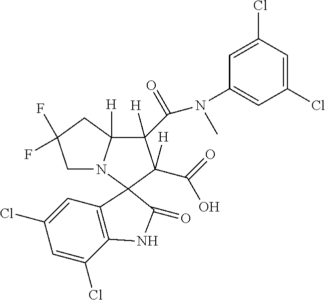 [SMILES: CN(C(=O)C1C2CC(F)(F)CN2C2(C(=O)Nc3c(Cl)cc(Cl)cc32)C1C(=O)O)c1cc(Cl)cc(Cl)c1] |
| 58 | 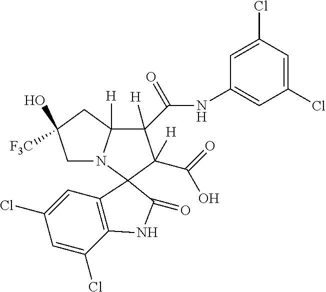 [SMILES: O=C(Nc1cc(Cl)cc(Cl)c1)C1C2C[C@@](O)(C(F)(F)F)CN2C2(C(=O)Nc3c(Cl)cc(Cl)cc32)C1C(=O)O] |
| 59 | 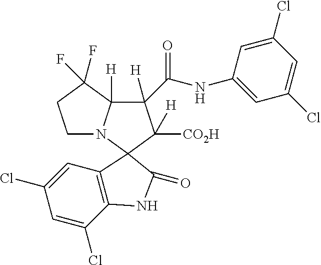 [SMILES: O=C(Nc1cc(Cl)cc(Cl)c1)C1C2N(CCC2(F)F)C2(C(=O)Nc3c(Cl)cc(Cl)cc32)C1C(=O)O] |
| 60 | 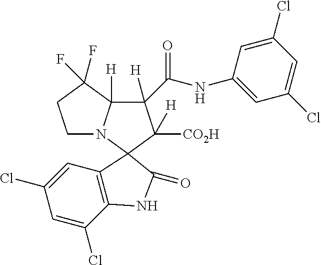 [SMILES: O=C(Nc1cc(Cl)cc(Cl)c1)C1C2N(CCC2(F)F)C2(C(=O)Nc3c(Cl)cc(Cl)cc32)C1C(=O)O] |
| 61 | 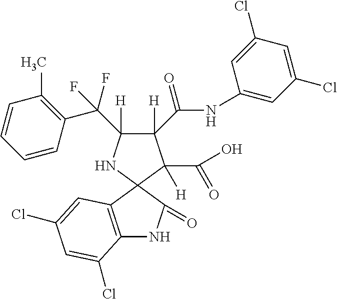 [SMILES: Cc1ccccc1C(F)(F)C1NC2(C(=O)Nc3c(Cl)cc(Cl)cc32)C(C(=O)O)C1C(=O)Nc1cc(Cl)cc(Cl)c1] |
| 62 | 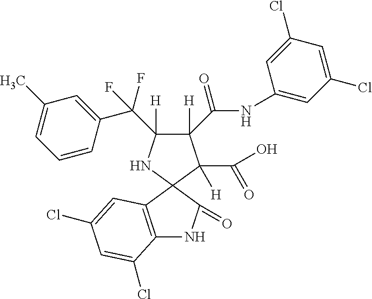 [SMILES: Cc1cccc(C(F)(F)C2NC3(C(=O)Nc4c(Cl)cc(Cl)cc43)C(C(=O)O)C2C(=O)Nc2cc(Cl)cc(Cl)c2)c1] |
| 63 | 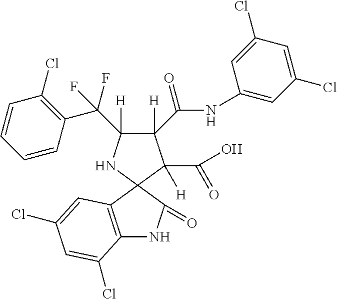 [SMILES: O=C(Nc1cc(Cl)cc(Cl)c1)C1C(C(F)(F)c2ccccc2Cl)NC2(C(=O)Nc3c(Cl)cc(Cl)cc32)C1C(=O)O] |
| 64 | 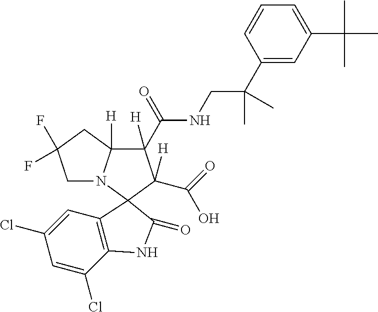 [SMILES: CC(C)(C)c1cccc(C(C)(C)CNC(=O)C2C3CC(F)(F)CN3C3(C(=O)Nc4c(Cl)cc(Cl)cc43)C2C(=O)O)c1] |
| 65 | 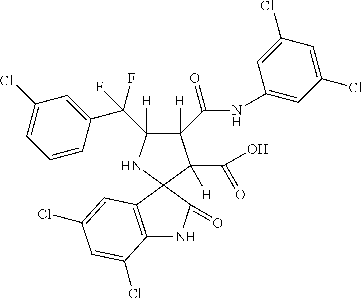 [SMILES: O=C(Nc1cc(Cl)cc(Cl)c1)C1C(C(F)(F)c2cccc(Cl)c2)NC2(C(=O)Nc3c(Cl)cc(Cl)cc32)C1C(=O)O] |
| 66.9a 66.9b.1 66.9b.2 66.9b.3 | 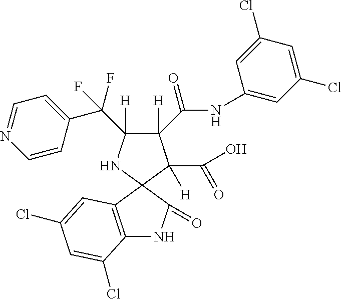 [SMILES: O=C(Nc1cc(Cl)cc(Cl)c1)C1C(C(F)(F)c2ccncc2)NC2(C(=O)Nc3c(Cl)cc(Cl)cc32)C1C(=O)O] |
| 67.9a 67.9b | 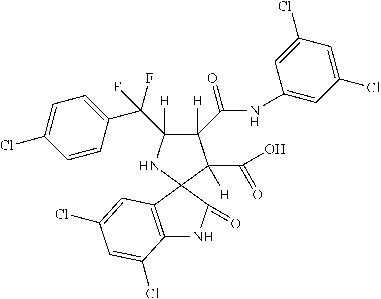 [SMILES: O=C(Nc1cc(Cl)cc(Cl)c1)C1C(C(F)(F)c2ccc(Cl)cc2)NC2(C(=O)Nc3c(Cl)cc(Cl)cc32)C1C(=O)O] |
| 68.a 68.b 68.c | 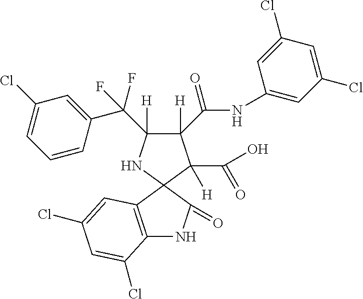 [SMILES: O=C(Nc1cc(Cl)cc(Cl)c1)C1C(C(F)(F)c2cccc(Cl)c2)NC2(C(=O)Nc3c(Cl)cc(Cl)cc32)C1C(=O)O] |
| 69.a 69.b | 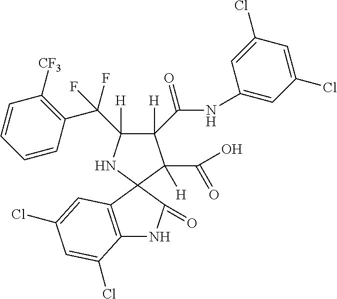 [SMILES: O=C(Nc1cc(Cl)cc(Cl)c1)C1C(C(F)(F)c2ccccc2C(F)(F)F)NC2(C(=O)Nc3c(Cl)cc(Cl)cc32)C1C(=O)O] |
| 70.a 70.b 70.c | 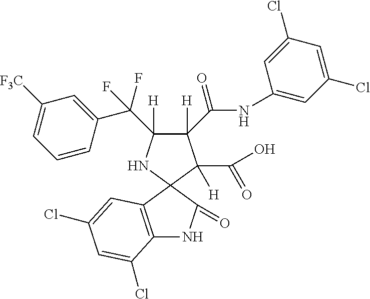 [SMILES: O=C(Nc1cc(Cl)cc(Cl)c1)C1C(C(F)(F)c2cccc(C(F)(F)F)c2)NC2(C(=O)Nc3c(Cl)cc(Cl)cc32)C1C(=O)O] |
| 71.a | 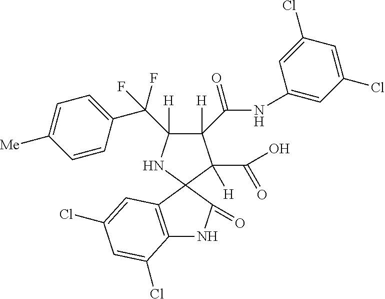 [SMILES: Cc1ccc(C(F)(F)C2NC3(C(=O)Nc4c(Cl)cc(Cl)cc43)C(C(=O)O)C2C(=O)Nc2cc(Cl)cc(Cl)c2)cc1] |
| 72.a 72.b 72.c | 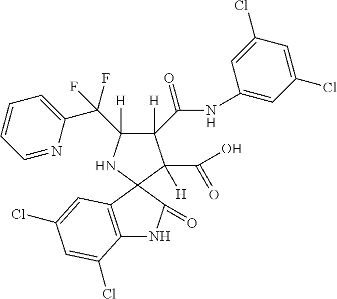 [SMILES: O=C(Nc1cc(Cl)cc(Cl)c1)C1C(C(F)(F)c2ccccn2)NC2(C(=O)Nc3c(Cl)cc(Cl)cc32)C1C(=O)O] |
| 73.a 73.b 73.c | 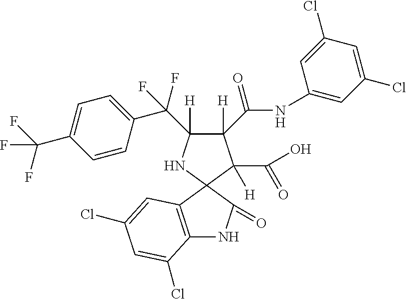 [SMILES: O=C(Nc1cc(Cl)cc(Cl)c1)C1C(C(F)(F)c2ccc(C(F)(F)F)cc2)NC2(C(=O)Nc3c(Cl)cc(Cl)cc32)C1C(=O)O] |
| 74.a | 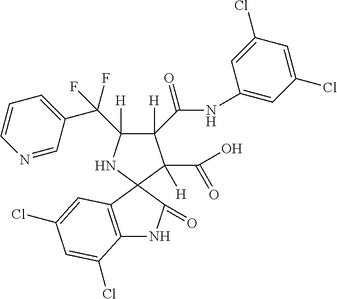 [SMILES: O=C(Nc1cc(Cl)cc(Cl)c1)C1C(C(F)(F)c2cccnc2)NC2(C(=O)Nc3c(Cl)cc(Cl)cc32)C1C(=O)O] |
| 75.a | 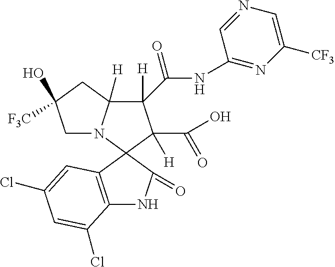 [SMILES: O=C(Nc1cncc(C(F)(F)F)n1)C1C2C[C@@](O)(C(F)(F)F)CN2C2(C(=O)Nc3c(Cl)cc(Cl)cc32)C1C(=O)O] |
| 76.a 76.b 76.c 76.d | 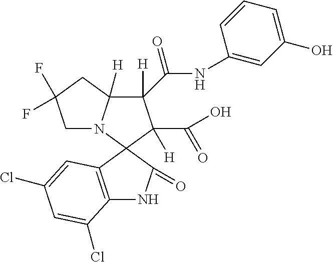 [SMILES: O=C(Nc1cccc(O)c1)C1C2CC(F)(F)CN2C2(C(=O)Nc3c(Cl)cc(Cl)cc32)C1C(=O)O] |
| 77.a 77.b | 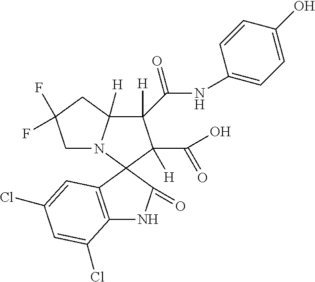 [SMILES: O=C(Nc1ccc(O)cc1)C1C2CC(F)(F)CN2C2(C(=O)Nc3c(Cl)cc(Cl)cc32)C1C(=O)O] |
| 78.a | 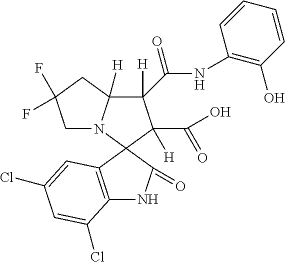 [SMILES: O=C(Nc1ccccc1O)C1C2CC(F)(F)CN2C2(C(=O)Nc3c(Cl)cc(Cl)cc32)C1C(=O)O] |
| 79a 79b 79c | 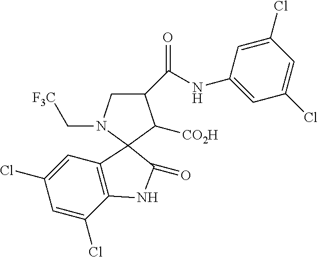 [SMILES: O=C(Nc1cc(Cl)cc(Cl)c1)C1CN(CC(F)(F)F)C2(C(=O)Nc3c(Cl)cc(Cl)cc32)C1C(=O)O] |
| 80.7a.1 80.7a.2 80.7b.1 80.7b.2 | 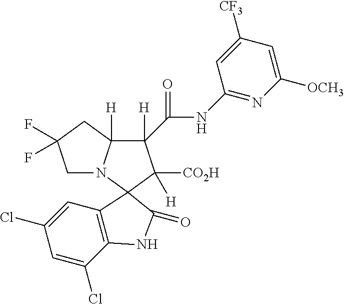 [SMILES: COc1cc(C(F)(F)F)cc(NC(=O)C2C3CC(F)(F)CN3C3(C(=O)Nc4c(Cl)cc(Cl)cc43)C2C(=O)O)n1] |
| 81 | 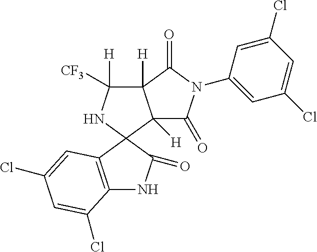 [SMILES: O=C1C2C(C(F)(F)F)NC3(C(=O)Nc4c(Cl)cc(Cl)cc43)C2C(=O)N1c1cc(Cl)cc(Cl)c1] |
| 82.6a | 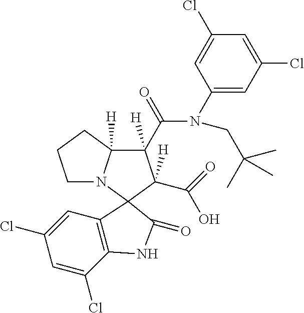 [SMILES: CC(C)(C)CN(C(=O)[C@H]1[C@@H](C(=O)O)C2(C(=O)Nc3c(Cl)cc(Cl)cc32)N2CCC[C@@H]12)c1cc(Cl)cc(Cl)c1] |
| 82.6b | 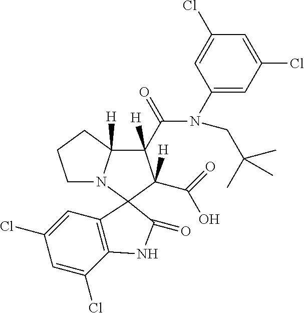 [SMILES: CC(C)(C)CN(C(=O)[C@H]1[C@H]2CCCN2C2(C(=O)Nc3c(Cl)cc(Cl)cc32)[C@H]1C(=O)O)c1cc(Cl)cc(Cl)c1] |
| 83a | 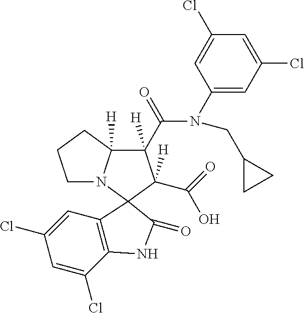 [SMILES: O=C(O)[C@@H]1[C@H](C(=O)N(CC2CC2)c2cc(Cl)cc(Cl)c2)[C@@H]2CCCN2C12C(=O)Nc1c(Cl)cc(Cl)cc12] |
| 83b | 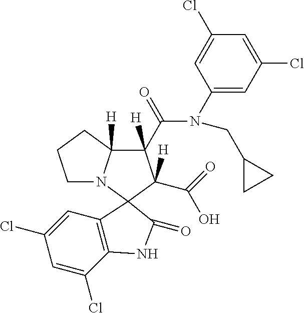 [SMILES: O=C(O)[C@H]1[C@@H](C(=O)N(CC2CC2)c2cc(Cl)cc(Cl)c2)[C@H]2CCCN2C12C(=O)Nc1c(Cl)cc(Cl)cc12] |
| 84a | 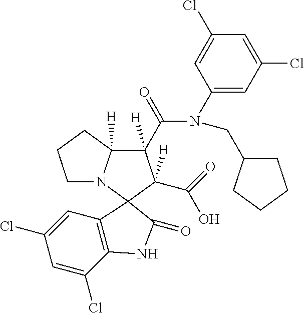 [SMILES: O=C(O)[C@@H]1[C@H](C(=O)N(CC2CCCC2)c2cc(Cl)cc(Cl)c2)[C@@H]2CCCN2C12C(=O)Nc1c(Cl)cc(Cl)cc12] |
| 84b | 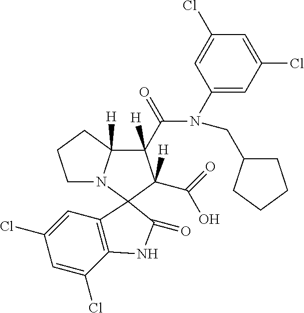 [SMILES: O=C(O)[C@H]1[C@@H](C(=O)N(CC2CCCC2)c2cc(Cl)cc(Cl)c2)[C@H]2CCCN2C12C(=O)Nc1c(Cl)cc(Cl)cc12] |
| 85a | 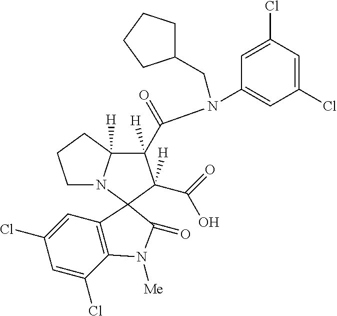 [SMILES: CN1C(=O)C2(c3cc(Cl)cc(Cl)c31)[C@H](C(=O)O)[C@H](C(=O)N(CC1CCCC1)c1cc(Cl)cc(Cl)c1)[C@@H]1CCCN12] |
| 85b | 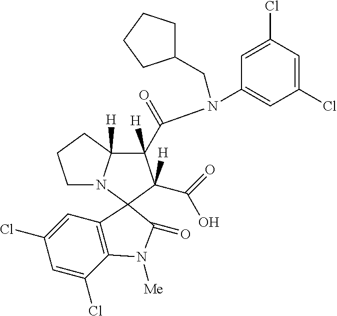 [SMILES: CN1C(=O)C2(c3cc(Cl)cc(Cl)c31)[C@@H](C(=O)O)[C@@H](C(=O)N(CC1CCCC1)c1cc(Cl)cc(Cl)c1)[C@H]1CCCN12] |
| 90 | 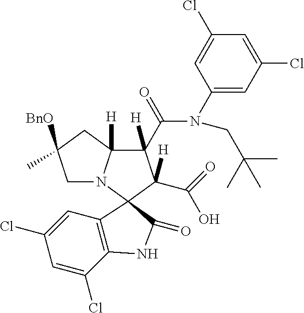 [SMILES: CC(C)(C)CN(C(=O)[C@H]1[C@H]2C[C@](C)(OCc3ccccc3)CN2[C@]2(C(=O)Nc3c(Cl)cc(Cl)cc32)[C@H]1C(=O)O)c1cc(Cl)cc(Cl)c1] |
| 91 | 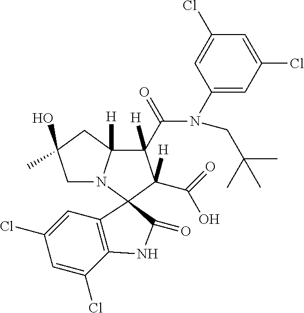 [SMILES: CC(C)(C)CN(C(=O)[C@H]1[C@H]2C[C@](C)(O)CN2[C@]2(C(=O)Nc3c(Cl)cc(Cl)cc32)[C@H]1C(=O)O)c1cc(Cl)cc(Cl)c1] |
| 92 | 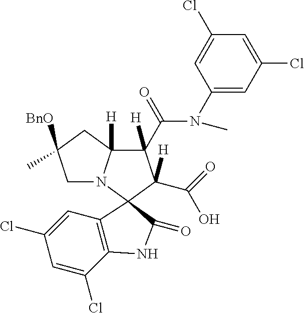 [SMILES: CN(C(=O)[C@H]1[C@H]2C[C@](C)(OCc3ccccc3)CN2[C@]2(C(=O)Nc3c(Cl)cc(Cl)cc32)[C@H]1C(=O)O)c1cc(Cl)cc(Cl)c1] |
| 93 | 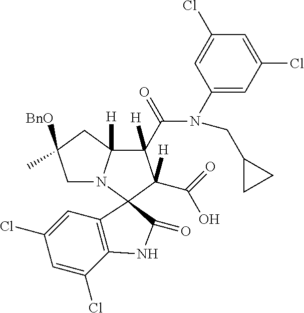 [SMILES: C[C@]1(OCc2ccccc2)C[C@@H]2[C@H](C(=O)N(CC3CC3)c3cc(Cl)cc(Cl)c3)[C@H](C(=O)O)[C@@]3(C(=O)Nc4c(Cl)cc(Cl)cc43)N2C1] |
| 94 | 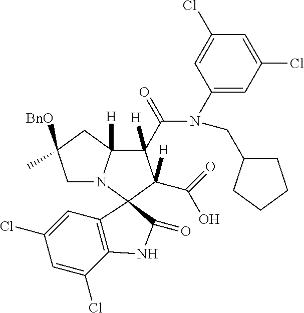 [SMILES: C[C@]1(OCc2ccccc2)C[C@@H]2[C@H](C(=O)N(CC3CCCC3)c3cc(Cl)cc(Cl)c3)[C@H](C(=O)O)[C@@]3(C(=O)Nc4c(Cl)cc(Cl)cc43)N2C1] |
| 95 | 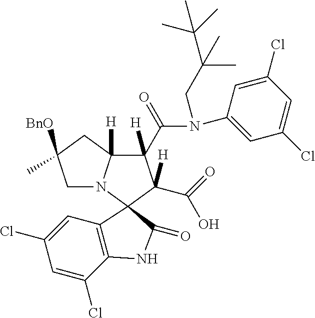 [SMILES: CC(C)(C)C(C)(C)CN(C(=O)[C@H]1[C@H]2C[C@](C)(OCc3ccccc3)CN2[C@]2(C(=O)Nc3c(Cl)cc(Cl)cc32)[C@H]1C(=O)O)c1cc(Cl)cc(Cl)c1] |
| 96 | 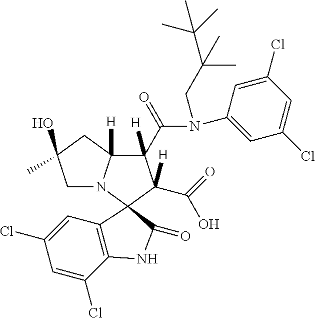 [SMILES: CC(C)(C)C(C)(C)CN(C(=O)[C@H]1[C@H]2C[C@](C)(O)CN2[C@]2(C(=O)Nc3c(Cl)cc(Cl)cc32)[C@H]1C(=O)O)c1cc(Cl)cc(Cl)c1] |
| 97 | 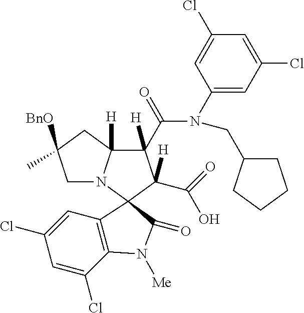 [SMILES: CN1C(=O)[C@]2(c3cc(Cl)cc(Cl)c31)[C@@H](C(=O)O)[C@@H](C(=O)N(CC1CCCC1)c1cc(Cl)cc(Cl)c1)[C@H]1C[C@](C)(OCc3ccccc3)CN12] |
| 100.6b | 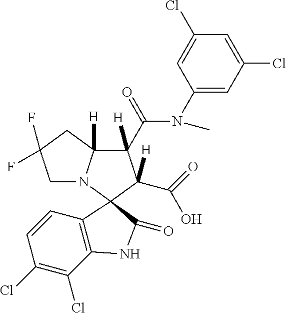 [SMILES: CN(C(=O)[C@H]1[C@H]2CC(F)(F)CN2[C@]2(C(=O)Nc3c2ccc(Cl)c3Cl)[C@H]1C(=O)O)c1cc(Cl)cc(Cl)c1] |
| 101 | 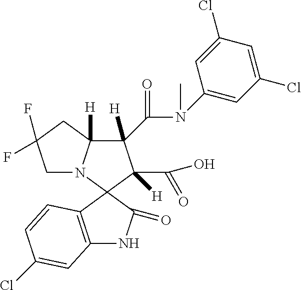 [SMILES: CN(C(=O)[C@H]1[C@H]2CC(F)(F)CN2C2(C(=O)Nc3cc(Cl)ccc32)[C@H]1C(=O)O)c1cc(Cl)cc(Cl)c1] |
| 102 | 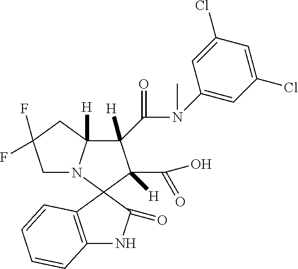 [SMILES: CN(C(=O)[C@H]1[C@H]2CC(F)(F)CN2C2(C(=O)Nc3ccccc32)[C@H]1C(=O)O)c1cc(Cl)cc(Cl)c1] |
| 103 | 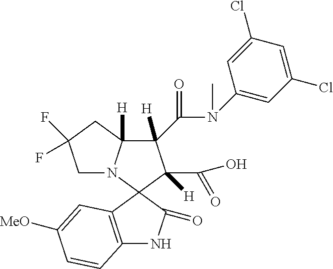 [SMILES: COc1ccc2c(c1)C1(C(=O)N2)[C@@H](C(=O)O)[C@@H](C(=O)N(C)c2cc(Cl)cc(Cl)c2)[C@H]2CC(F)(F)CN21] |
| 104 | 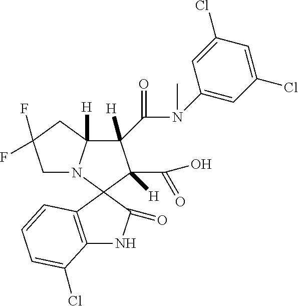 [SMILES: CN(C(=O)[C@H]1[C@H]2CC(F)(F)CN2C2(C(=O)Nc3c(Cl)cccc32)[C@H]1C(=O)O)c1cc(Cl)cc(Cl)c1] |
| 110 | 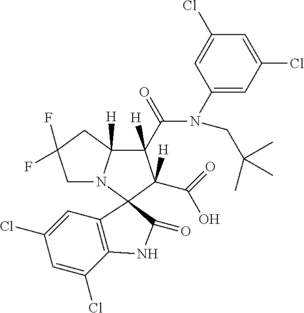 [SMILES: CC(C)(C)CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 111 | 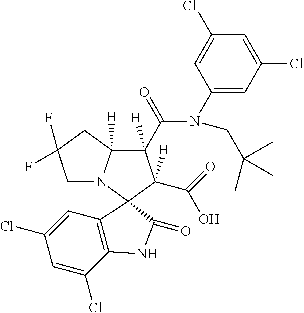 [SMILES: CC(C)(C)CN(C(=O)[C@H]1[C@@H](C(=O)O)[C@]2(C(=O)Nc3c(Cl)cc(Cl)cc32)N2CC(F)(F)C[C@@H]12)c1cc(Cl)cc(Cl)c1] |
| 112 | 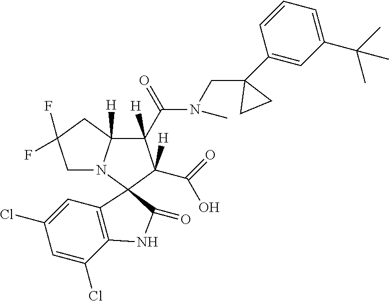 [SMILES: CN(CC1(c2cccc(C(C)(C)C)c2)CC1)C(=O)[C@H]1[C@H]2CC(F)(F)CN2[C@]2(C(=O)Nc3c(Cl)cc(Cl)cc32)[C@H]1C(=O)O] |
| 113 | 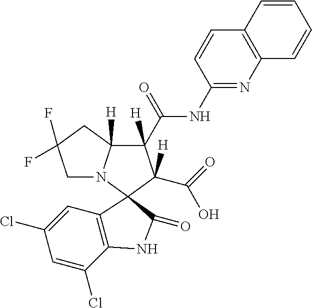 [SMILES: O=C(Nc1ccc2ccccc2n1)[C@H]1[C@H]2CC(F)(F)CN2[C@]2(C(=O)Nc3c(Cl)cc(Cl)cc32)[C@H]1C(=O)O] |
| 114 | 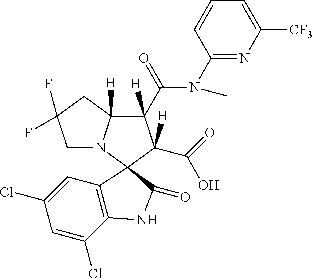 [SMILES: CN(C(=O)[C@H]1[C@H]2CC(F)(F)CN2[C@]2(C(=O)Nc3c(Cl)cc(Cl)cc32)[C@H]1C(=O)O)c1cccc(C(F)(F)F)n1] |
| 115 | 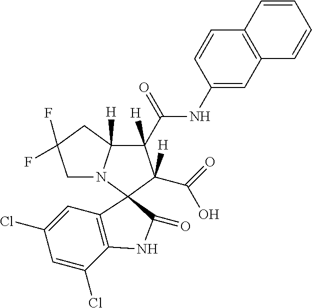 [SMILES: O=C(Nc1ccc2ccccc2c1)[C@H]1[C@H]2CC(F)(F)CN2[C@]2(C(=O)Nc3c(Cl)cc(Cl)cc32)[C@H]1C(=O)O] |
| 116 | 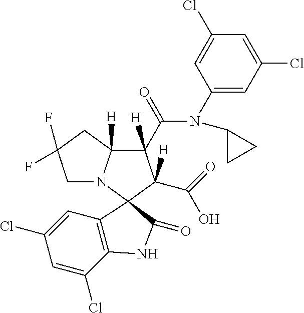 [SMILES: O=C(O)[C@H]1[C@@H](C(=O)N(c2cc(Cl)cc(Cl)c2)C2CC2)[C@H]2CC(F)(F)CN2[C@]12C(=O)Nc1c(Cl)cc(Cl)cc12] |
| 117 | 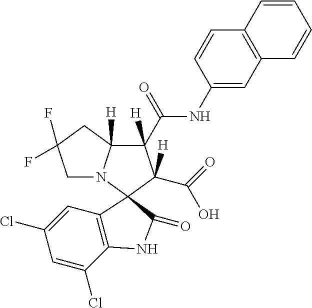 [SMILES: O=C(Nc1ccc2ccccc2c1)[C@H]1[C@H]2CC(F)(F)CN2[C@]2(C(=O)Nc3c(Cl)cc(Cl)cc32)[C@H]1C(=O)O] |
| 118 | 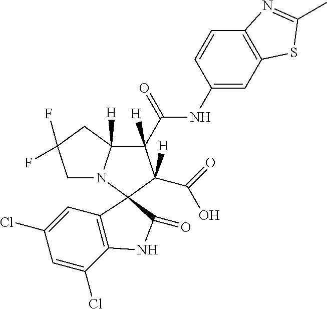 [SMILES: Cc1nc2ccc(NC(=O)[C@H]3[C@H]4CC(F)(F)CN4[C@]4(C(=O)Nc5c(Cl)cc(Cl)cc54)[C@H]3C(=O)O)cc2s1] |
| 119 | 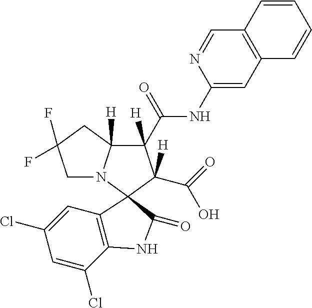 [SMILES: O=C(Nc1cc2ccccc2cn1)[C@H]1[C@H]2CC(F)(F)CN2[C@]2(C(=O)Nc3c(Cl)cc(Cl)cc32)[C@H]1C(=O)O] |
| 120 | 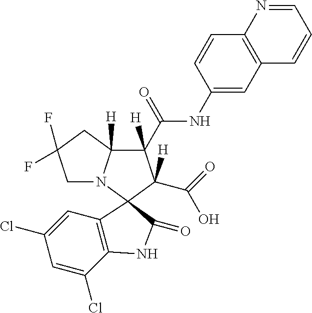 [SMILES: O=C(Nc1ccc2ncccc2c1)[C@H]1[C@H]2CC(F)(F)CN2[C@]2(C(=O)Nc3c(Cl)cc(Cl)cc32)[C@H]1C(=O)O] |
| 121 | 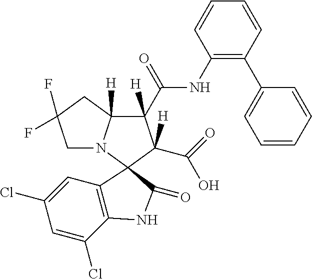 [SMILES: O=C(Nc1ccccc1-c1ccccc1)[C@H]1[C@H]2CC(F)(F)CN2[C@]2(C(=O)Nc3c(Cl)cc(Cl)cc32)[C@H]1C(=O)O] |
| 122 | 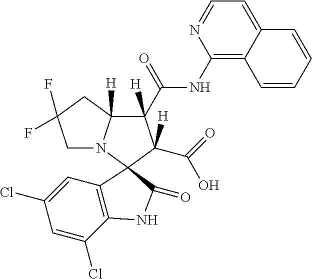 [SMILES: O=C(Nc1nccc2ccccc12)[C@H]1[C@H]2CC(F)(F)CN2[C@]2(C(=O)Nc3c(Cl)cc(Cl)cc32)[C@H]1C(=O)O] |
| 123 | 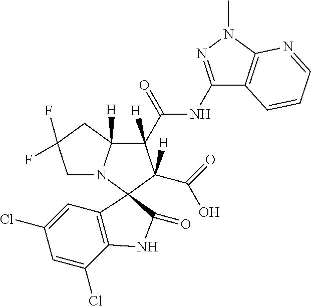 [SMILES: Cn1nc(NC(=O)[C@H]2[C@H]3CC(F)(F)CN3[C@]3(C(=O)Nc4c(Cl)cc(Cl)cc43)[C@H]2C(=O)O)c2cccnc21] |
| 124 | 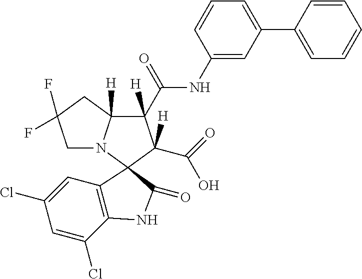 [SMILES: O=C(Nc1cccc(-c2ccccc2)c1)[C@H]1[C@H]2CC(F)(F)CN2[C@]2(C(=O)Nc3c(Cl)cc(Cl)cc32)[C@H]1C(=O)O] |
| 125 | 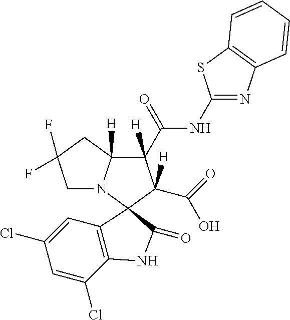 [SMILES: O=C(Nc1nc2ccccc2s1)[C@H]1[C@H]2CC(F)(F)CN2[C@]2(C(=O)Nc3c(Cl)cc(Cl)cc32)[C@H]1C(=O)O] |
| 126 | 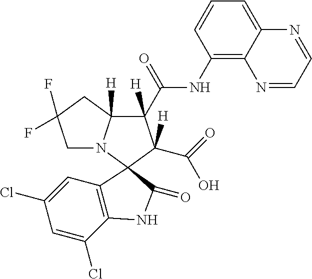 [SMILES: O=C(Nc1cccc2nccnc12)[C@H]1[C@H]2CC(F)(F)CN2[C@]2(C(=O)Nc3c(Cl)cc(Cl)cc32)[C@H]1C(=O)O] |
| 127 | 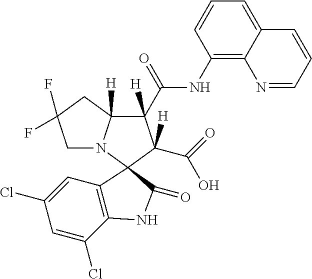 [SMILES: O=C(Nc1cccc2cccnc12)[C@H]1[C@H]2CC(F)(F)CN2[C@]2(C(=O)Nc3c(Cl)cc(Cl)cc32)[C@H]1C(=O)O] |
| 128 | 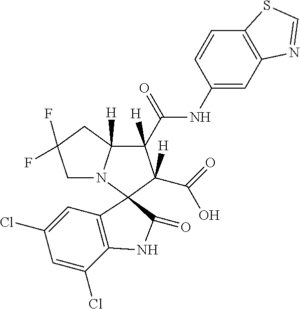 [SMILES: O=C(Nc1ccc2scnc2c1)[C@H]1[C@H]2CC(F)(F)CN2[C@]2(C(=O)Nc3c(Cl)cc(Cl)cc32)[C@H]1C(=O)O] |
| 129 | 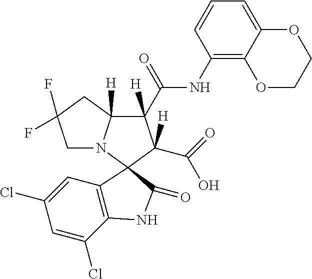 [SMILES: O=C(Nc1cccc2c1OCCO2)[C@H]1[C@H]2CC(F)(F)CN2[C@]2(C(=O)Nc3c(Cl)cc(Cl)cc32)[C@H]1C(=O)O] |
| 130 | 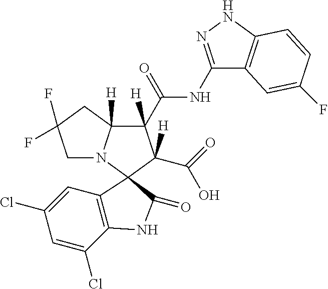 [SMILES: O=C(Nc1n[nH]c2ccc(F)cc12)[C@H]1[C@H]2CC(F)(F)CN2[C@]2(C(=O)Nc3c(Cl)cc(Cl)cc32)[C@H]1C(=O)O] |
| 131 | 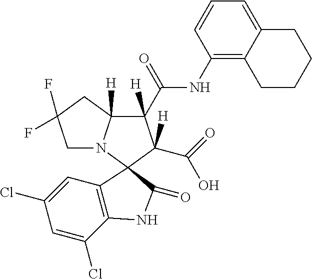 [SMILES: O=C(Nc1cccc2c1CCCC2)[C@H]1[C@H]2CC(F)(F)CN2[C@]2(C(=O)Nc3c(Cl)cc(Cl)cc32)[C@H]1C(=O)O] |
| 132 | 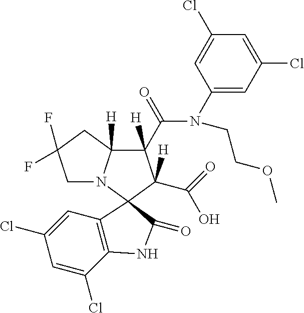 [SMILES: COCCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 133 | 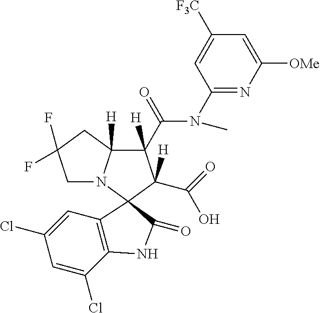 [SMILES: COc1cc(C(F)(F)F)cc(N(C)C(=O)[C@H]2[C@H]3CC(F)(F)CN3[C@]3(C(=O)Nc4c(Cl)cc(Cl)cc43)[C@H]2C(=O)O)n1] |
| 134 | 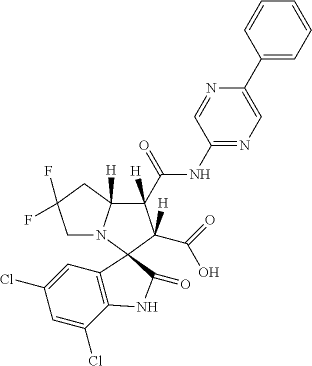 [SMILES: O=C(Nc1cnc(-c2ccccc2)cn1)[C@H]1[C@H]2CC(F)(F)CN2[C@]2(C(=O)Nc3c(Cl)cc(Cl)cc32)[C@H]1C(=O)O] |
| 135 | 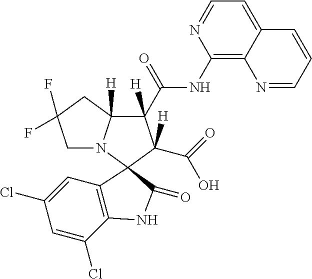 [SMILES: O=C(Nc1nccc2cccnc12)[C@H]1[C@H]2CC(F)(F)CN2[C@]2(C(=O)Nc3c(Cl)cc(Cl)cc32)[C@H]1C(=O)O] |
| 136 | 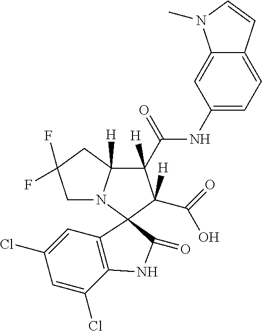 [SMILES: Cn1ccc2ccc(NC(=O)[C@H]3[C@H]4CC(F)(F)CN4[C@]4(C(=O)Nc5c(Cl)cc(Cl)cc54)[C@H]3C(=O)O)cc21] |
| 137 | 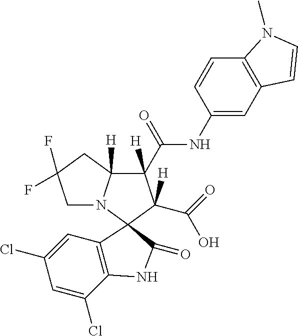 [SMILES: Cn1ccc2cc(NC(=O)[C@H]3[C@H]4CC(F)(F)CN4[C@]4(C(=O)Nc5c(Cl)cc(Cl)cc54)[C@H]3C(=O)O)ccc21] |
| 138 | 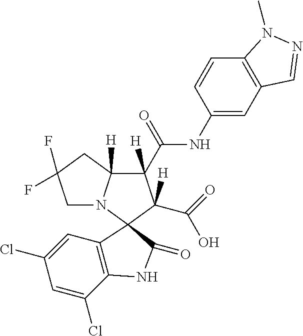 [SMILES: Cn1ncc2cc(NC(=O)[C@H]3[C@H]4CC(F)(F)CN4[C@]4(C(=O)Nc5c(Cl)cc(Cl)cc54)[C@H]3C(=O)O)ccc21] |
| 139 | 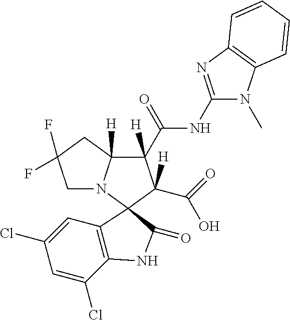 [SMILES: Cn1c(NC(=O)[C@H]2[C@H]3CC(F)(F)CN3[C@]3(C(=O)Nc4c(Cl)cc(Cl)cc43)[C@H]2C(=O)O)nc2ccccc21] |
| 140 | 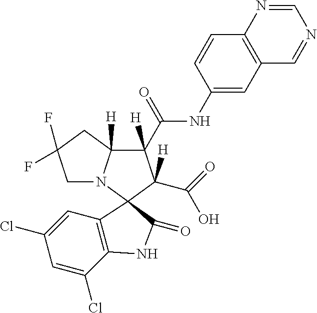 [SMILES: O=C(Nc1ccc2ncncc2c1)[C@H]1[C@H]2CC(F)(F)CN2[C@]2(C(=O)Nc3c(Cl)cc(Cl)cc32)[C@H]1C(=O)O] |
| 141 | 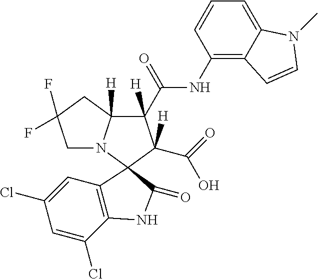 [SMILES: Cn1ccc2c(NC(=O)[C@H]3[C@H]4CC(F)(F)CN4[C@]4(C(=O)Nc5c(Cl)cc(Cl)cc54)[C@H]3C(=O)O)cccc21] |
| 142 | 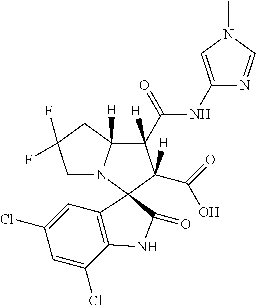 [SMILES: Cn1cnc(NC(=O)[C@H]2[C@H]3CC(F)(F)CN3[C@]3(C(=O)Nc4c(Cl)cc(Cl)cc43)[C@H]2C(=O)O)c1] |
| 143 | 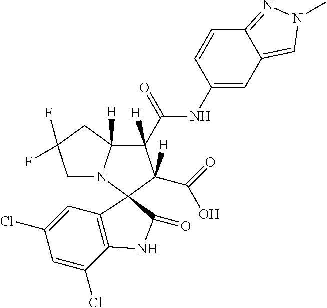 [SMILES: Cn1cc2cc(NC(=O)[C@H]3[C@H]4CC(F)(F)CN4[C@]4(C(=O)Nc5c(Cl)cc(Cl)cc54)[C@H]3C(=O)O)ccc2n1] |
| 144 | 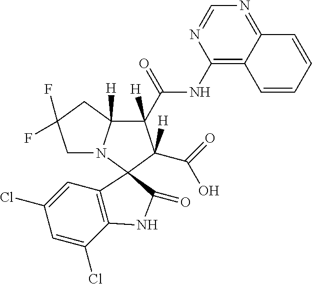 [SMILES: O=C(Nc1ncnc2ccccc12)[C@H]1[C@H]2CC(F)(F)CN2[C@]2(C(=O)Nc3c(Cl)cc(Cl)cc32)[C@H]1C(=O)O] |
| 145 | 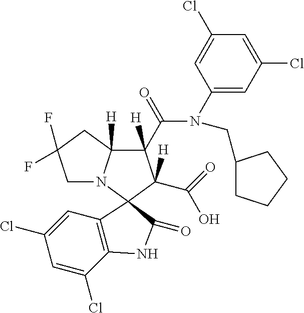 [SMILES: O=C(O)[C@H]1[C@@H](C(=O)N(CC2CCCC2)c2cc(Cl)cc(Cl)c2)[C@H]2CC(F)(F)CN2[C@]12C(=O)Nc1c(Cl)cc(Cl)cc12] |
| 146 | 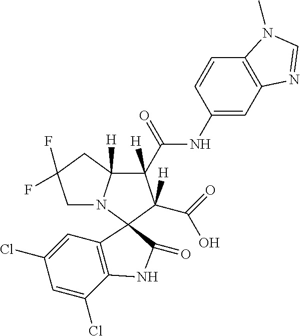 [SMILES: Cn1cnc2cc(NC(=O)[C@H]3[C@H]4CC(F)(F)CN4[C@]4(C(=O)Nc5c(Cl)cc(Cl)cc54)[C@H]3C(=O)O)ccc21] |
| 147 | 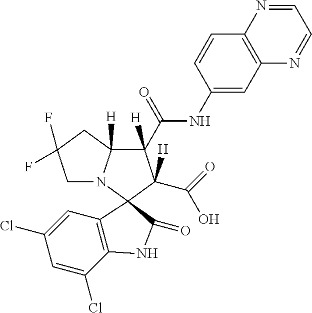 [SMILES: O=C(Nc1ccc2nccnc2c1)[C@H]1[C@H]2CC(F)(F)CN2[C@]2(C(=O)Nc3c(Cl)cc(Cl)cc32)[C@H]1C(=O)O] |
| 148 | 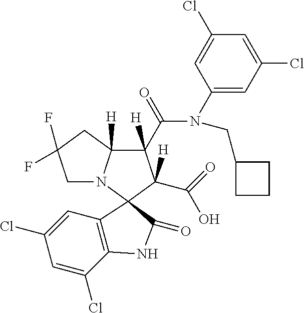 [SMILES: O=C(O)[C@H]1[C@@H](C(=O)N(CC2CCC2)c2cc(Cl)cc(Cl)c2)[C@H]2CC(F)(F)CN2[C@]12C(=O)Nc1c(Cl)cc(Cl)cc12] |
| 149 | 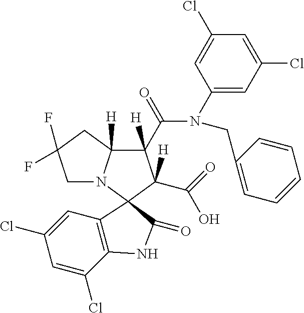 [SMILES: O=C(O)[C@H]1[C@@H](C(=O)N(Cc2ccccc2)c2cc(Cl)cc(Cl)c2)[C@H]2CC(F)(F)CN2[C@]12C(=O)Nc1c(Cl)cc(Cl)cc12] |
| 150 | 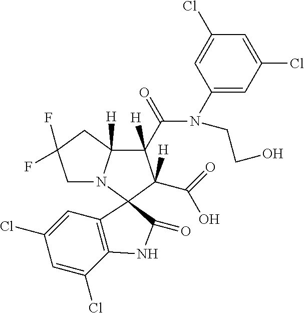 [SMILES: O=C(O)[C@H]1[C@@H](C(=O)N(CCO)c2cc(Cl)cc(Cl)c2)[C@H]2CC(F)(F)CN2[C@]12C(=O)Nc1c(Cl)cc(Cl)cc12] |
| 151 | 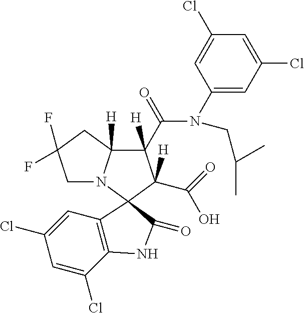 [SMILES: CC(C)CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 152 | 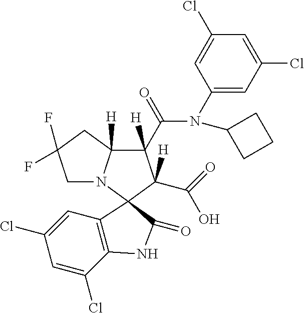 [SMILES: O=C(O)[C@H]1[C@@H](C(=O)N(c2cc(Cl)cc(Cl)c2)C2CCC2)[C@H]2CC(F)(F)CN2[C@]12C(=O)Nc1c(Cl)cc(Cl)cc12] |
| 153 | 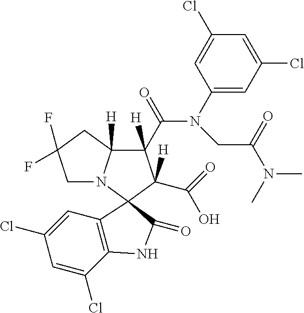 [SMILES: CN(C)C(=O)CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 154 | 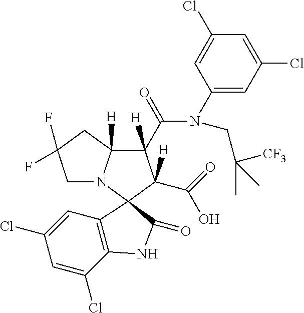 [SMILES: CC(C)(CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1)C(F)(F)F] |
| 155 | 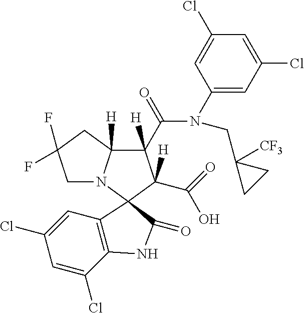 [SMILES: O=C(O)[C@H]1[C@@H](C(=O)N(CC2(C(F)(F)F)CC2)c2cc(Cl)cc(Cl)c2)[C@H]2CC(F)(F)CN2[C@]12C(=O)Nc1c(Cl)cc(Cl)cc12] |
| 156 | 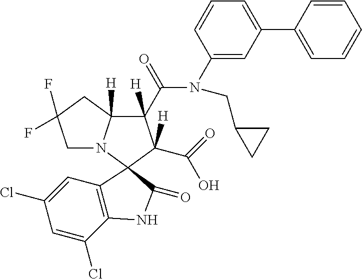 [SMILES: O=C(O)[C@H]1[C@@H](C(=O)N(CC2CC2)c2cccc(-c3ccccc3)c2)[C@H]2CC(F)(F)CN2[C@]12C(=O)Nc1c(Cl)cc(Cl)cc12] |
| 157 | 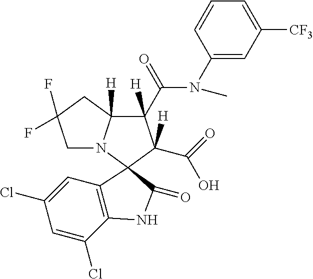 [SMILES: CN(C(=O)[C@H]1[C@H]2CC(F)(F)CN2[C@]2(C(=O)Nc3c(Cl)cc(Cl)cc32)[C@H]1C(=O)O)c1cccc(C(F)(F)F)c1] |
| 158 | 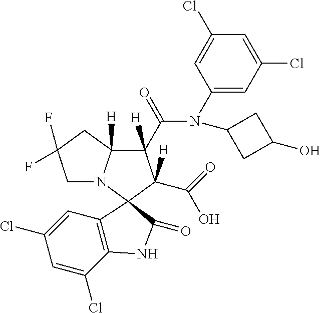 [SMILES: O=C(O)[C@H]1[C@@H](C(=O)N(c2cc(Cl)cc(Cl)c2)C2CC(O)C2)[C@H]2CC(F)(F)CN2[C@]12C(=O)Nc1c(Cl)cc(Cl)cc12] |
| 159 | 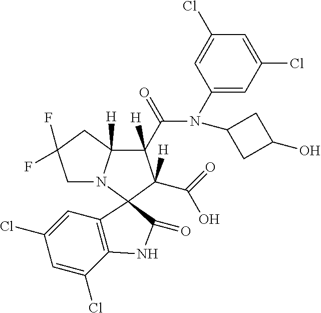 [SMILES: O=C(O)[C@H]1[C@@H](C(=O)N(c2cc(Cl)cc(Cl)c2)C2CC(O)C2)[C@H]2CC(F)(F)CN2[C@]12C(=O)Nc1c(Cl)cc(Cl)cc12] |
| 160 | 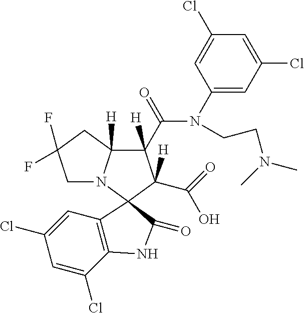 [SMILES: CN(C)CCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 161 | 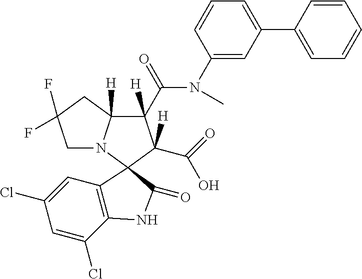 [SMILES: CN(C(=O)[C@H]1[C@H]2CC(F)(F)CN2[C@]2(C(=O)Nc3c(Cl)cc(Cl)cc32)[C@H]1C(=O)O)c1cccc(-c2ccccc2)c1] |
| 162 | 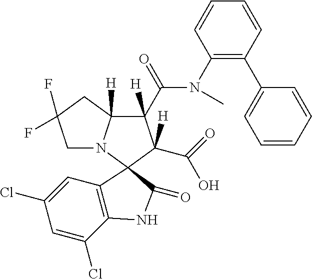 [SMILES: CN(C(=O)[C@H]1[C@H]2CC(F)(F)CN2[C@]2(C(=O)Nc3c(Cl)cc(Cl)cc32)[C@H]1C(=O)O)c1ccccc1-c1ccccc1] |
| 163 | 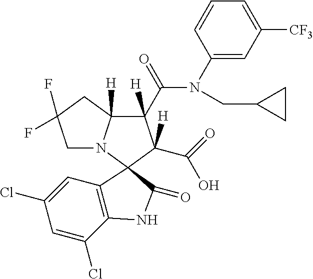 [SMILES: O=C(O)[C@H]1[C@@H](C(=O)N(CC2CC2)c2cccc(C(F)(F)F)c2)[C@H]2CC(F)(F)CN2[C@]12C(=O)Nc1c(Cl)cc(Cl)cc12] |
| 164 | 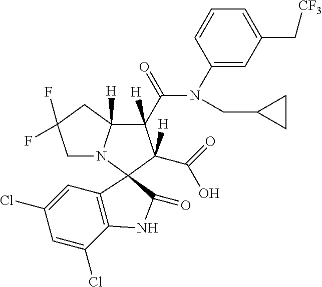 [SMILES: O=C(O)[C@H]1[C@@H](C(=O)N(CC2CC2)c2cccc(CC(F)(F)F)c2)[C@H]2CC(F)(F)CN2[C@]12C(=O)Nc1c(Cl)cc(Cl)cc12] |
| 165 | 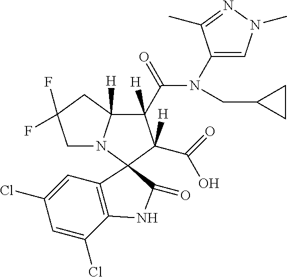 [SMILES: Cc1nn(C)cc1N(CC1CC1)C(=O)[C@H]1[C@H]2CC(F)(F)CN2[C@]2(C(=O)Nc3c(Cl)cc(Cl)cc32)[C@H]1C(=O)O] |
| 166 | 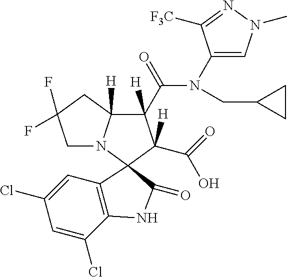 [SMILES: Cn1cc(N(CC2CC2)C(=O)[C@H]2[C@H]3CC(F)(F)CN3[C@]3(C(=O)Nc4c(Cl)cc(Cl)cc43)[C@H]2C(=O)O)c(C(F)(F)F)n1] |
| 167 | 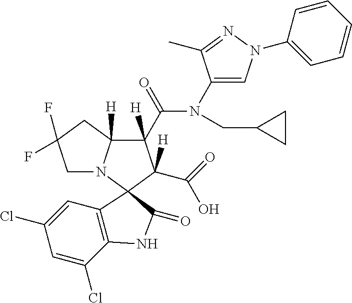 [SMILES: Cc1nn(-c2ccccc2)cc1N(CC1CC1)C(=O)[C@H]1[C@H]2CC(F)(F)CN2[C@]2(C(=O)Nc3c(Cl)cc(Cl)cc32)[C@H]1C(=O)O] |
| 168 | 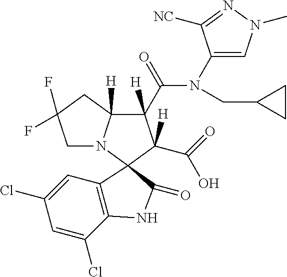 [SMILES: Cn1cc(N(CC2CC2)C(=O)[C@H]2[C@H]3CC(F)(F)CN3[C@]3(C(=O)Nc4c(Cl)cc(Cl)cc43)[C@H]2C(=O)O)c(C#N)n1] |
| 169 | 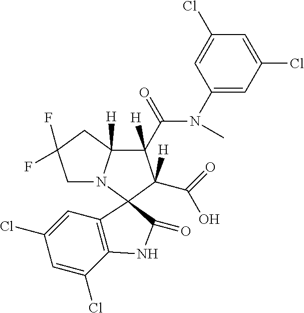 [SMILES: CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 170 | 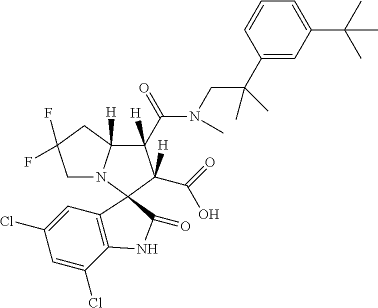 [SMILES: CN(CC(C)(C)c1cccc(C(C)(C)C)c1)C(=O)[C@H]1[C@H]2CC(F)(F)CN2[C@]2(C(=O)Nc3c(Cl)cc(Cl)cc32)[C@H]1C(=O)O] |
| 171 | 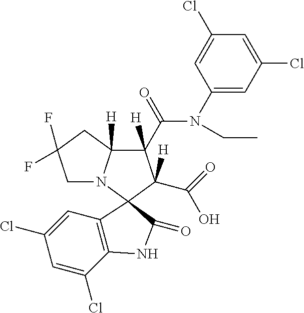 [SMILES: CCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 172 | 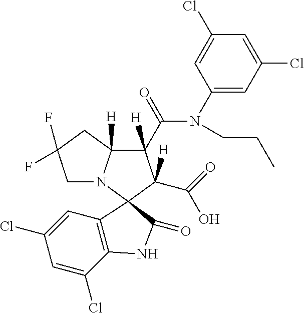 [SMILES: CCCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 173 | 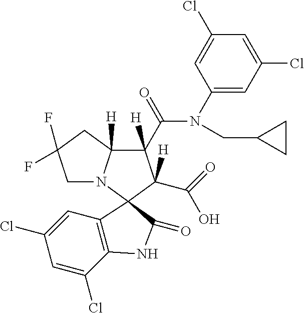 [SMILES: O=C(O)[C@H]1[C@@H](C(=O)N(CC2CC2)c2cc(Cl)cc(Cl)c2)[C@H]2CC(F)(F)CN2[C@]12C(=O)Nc1c(Cl)cc(Cl)cc12] |
| 174 | 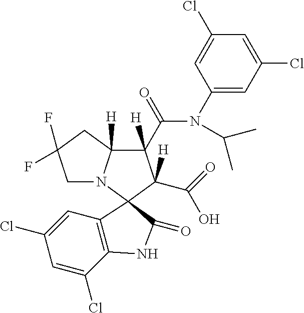 [SMILES: CC(C)N(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 175 | 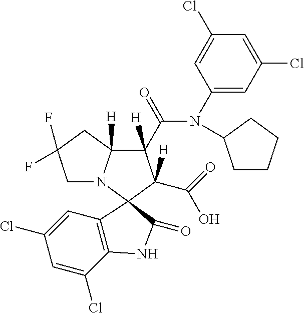 [SMILES: O=C(O)[C@H]1[C@@H](C(=O)N(c2cc(Cl)cc(Cl)c2)C2CCCC2)[C@H]2CC(F)(F)CN2[C@]12C(=O)Nc1c(Cl)cc(Cl)cc12] |
| 176 | 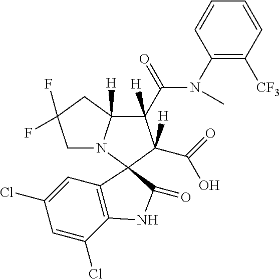 [SMILES: CN(C(=O)[C@H]1[C@H]2CC(F)(F)CN2[C@]2(C(=O)Nc3c(Cl)cc(Cl)cc32)[C@H]1C(=O)O)c1ccccc1C(F)(F)F] |
| 177 | 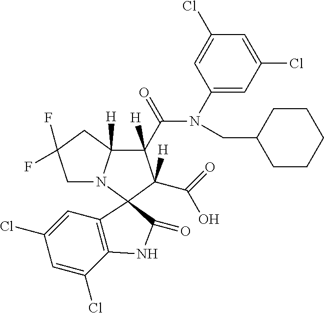 [SMILES: O=C(O)[C@H]1[C@@H](C(=O)N(CC2CCCCC2)c2cc(Cl)cc(Cl)c2)[C@H]2CC(F)(F)CN2[C@]12C(=O)Nc1c(Cl)cc(Cl)cc12] |
| 178 | 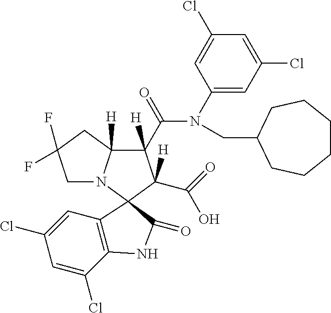 [SMILES: O=C(O)[C@H]1[C@@H](C(=O)N(CC2CCCCCC2)c2cc(Cl)cc(Cl)c2)[C@H]2CC(F)(F)CN2[C@]12C(=O)Nc1c(Cl)cc(Cl)cc12] |
| 179 | 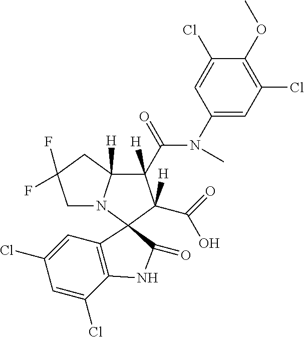 [SMILES: COc1c(Cl)cc(N(C)C(=O)[C@H]2[C@H]3CC(F)(F)CN3[C@]3(C(=O)Nc4c(Cl)cc(Cl)cc43)[C@H]2C(=O)O)cc1Cl] |
| 180 | 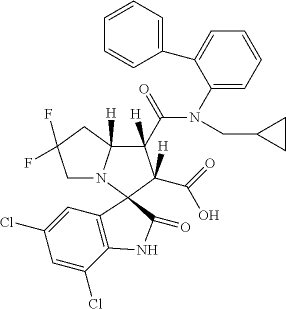 [SMILES: O=C(O)[C@H]1[C@@H](C(=O)N(CC2CC2)c2ccccc2-c2ccccc2)[C@H]2CC(F)(F)CN2[C@]12C(=O)Nc1c(Cl)cc(Cl)cc12] |
| 181 | 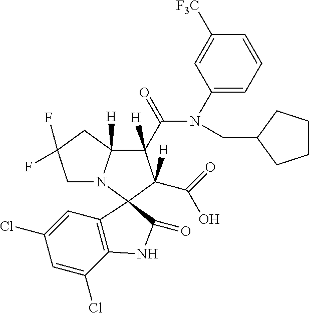 [SMILES: O=C(O)[C@H]1[C@@H](C(=O)N(CC2CCCC2)c2cccc(C(F)(F)F)c2)[C@H]2CC(F)(F)CN2[C@]12C(=O)Nc1c(Cl)cc(Cl)cc12] |
| 182 | 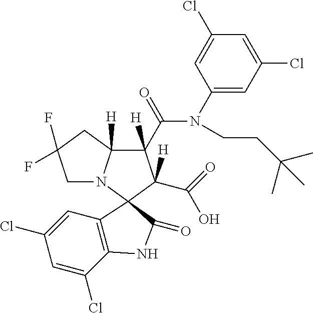 [SMILES: CC(C)(C)CCN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 183 | 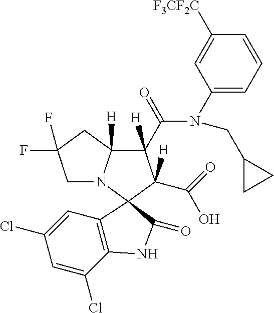 |
| 184 | 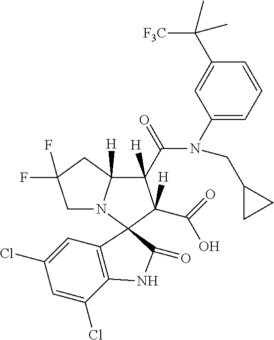 [SMILES: CC(C)(c1cccc(N(CC2CC2)C(=O)[C@H]2[C@H]3CC(F)(F)CN3[C@]3(C(=O)Nc4c(Cl)cc(Cl)cc43)[C@H]2C(=O)O)c1)C(F)(F)F] |
| 185 | 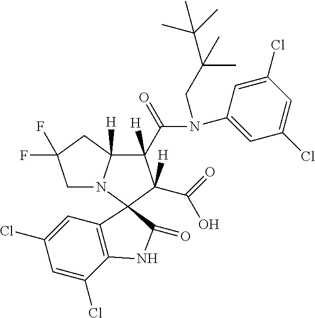 [SMILES: CC(C)(C)C(C)(C)CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1] |
| 186 | 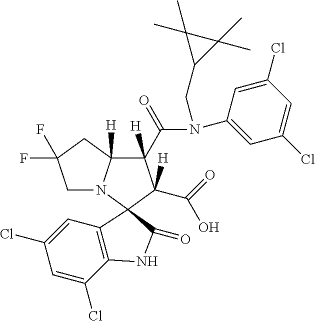 [SMILES: CC1(C)C(CN(C(=O)[C@H]2[C@H]3CC(F)(F)CN3[C@]3(C(=O)Nc4c(Cl)cc(Cl)cc43)[C@H]2C(=O)O)c2cc(Cl)cc(Cl)c2)C1(C)C] |
| 187 | 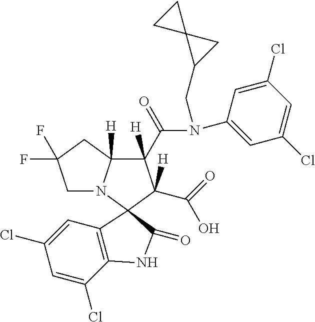 [SMILES: O=C(O)[C@H]1[C@@H](C(=O)N(CC2CC23CC3)c2cc(Cl)cc(Cl)c2)[C@H]2CC(F)(F)CN2[C@]12C(=O)Nc1c(Cl)cc(Cl)cc12] |
| 188 | 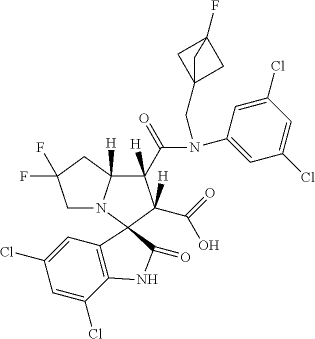 [SMILES: O=C(O)[C@H]1[C@@H](C(=O)N(CC23CC(F)(C2)C3)c2cc(Cl)cc(Cl)c2)[C@H]2CC(F)(F)CN2[C@]12C(=O)Nc1c(Cl)cc(Cl)cc12] |
| 189 | 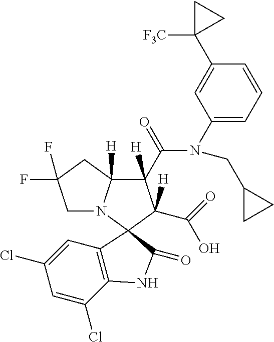 [SMILES: O=C(O)[C@H]1[C@@H](C(=O)N(CC2CC2)c2cccc(C3(C(F)(F)F)CC3)c2)[C@H]2CC(F)(F)CN2[C@]12C(=O)Nc1c(Cl)cc(Cl)cc12] |
| 190 | 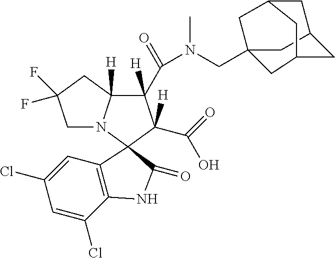 [SMILES: CN(CC12CC3CC(CC(C3)C1)C2)C(=O)[C@H]1[C@H]2CC(F)(F)CN2[C@]2(C(=O)Nc3c(Cl)cc(Cl)cc32)[C@H]1C(=O)O] |
| 191 | 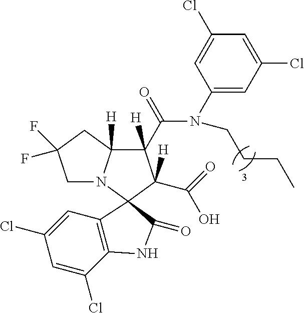 |
| 192 | 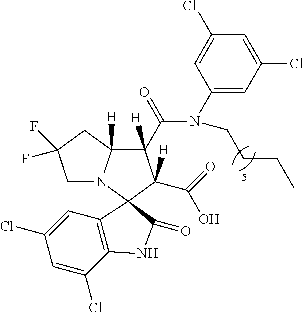 |
| 193 | 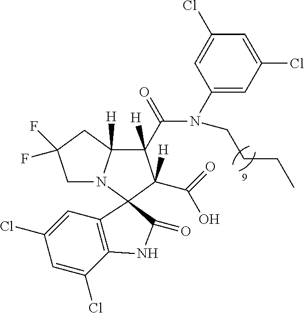 |
| 194 | 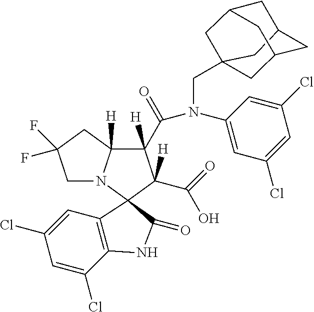 [SMILES: O=C(O)[C@H]1[C@@H](C(=O)N(CC23CC4CC(CC(C4)C2)C3)c2cc(Cl)cc(Cl)c2)[C@H]2CC(F)(F)CN2[C@]12C(=O)Nc1c(Cl)cc(Cl)cc12] |
| 195 | 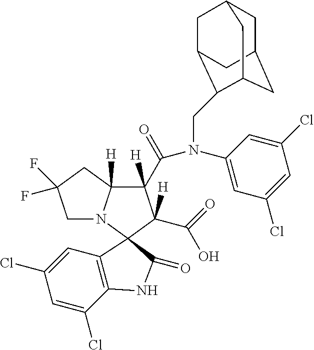 [SMILES: O=C(O)[C@H]1[C@@H](C(=O)N(CC2C3CC4CC(C3)CC2C4)c2cc(Cl)cc(Cl)c2)[C@H]2CC(F)(F)CN2[C@]12C(=O)Nc1c(Cl)cc(Cl)cc12] |
| 196 | 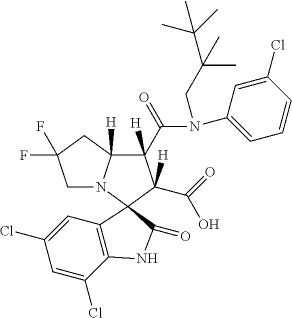 [SMILES: CC(C)(C)C(C)(C)CN(C(=O)[C@H]1[C@H]2CC(F)(F)CN2[C@]2(C(=O)Nc3c(Cl)cc(Cl)cc32)[C@H]1C(=O)O)c1cccc(Cl)c1] |
| 197 | 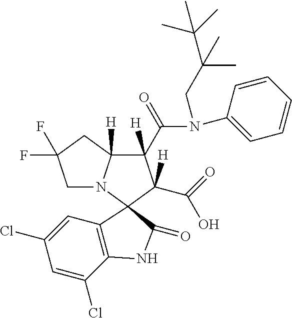 [SMILES: CC(C)(C)C(C)(C)CN(C(=O)[C@H]1[C@H]2CC(F)(F)CN2[C@]2(C(=O)Nc3c(Cl)cc(Cl)cc32)[C@H]1C(=O)O)c1ccccc1] |
| 198 | 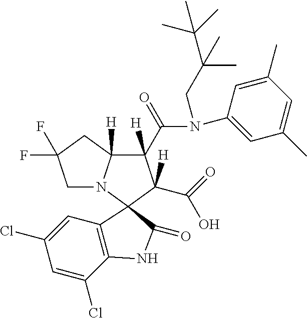 [SMILES: Cc1cc(C)cc(N(CC(C)(C)C(C)(C)C)C(=O)[C@H]2[C@H]3CC(F)(F)CN3[C@]3(C(=O)Nc4c(Cl)cc(Cl)cc43)[C@H]2C(=O)O)c1] |
| 199 | 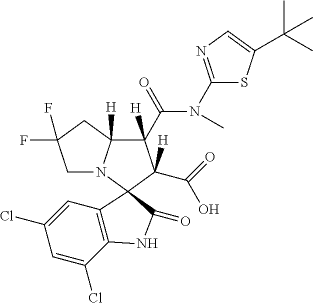 [SMILES: CN(C(=O)[C@H]1[C@H]2CC(F)(F)CN2[C@]2(C(=O)Nc3c(Cl)cc(Cl)cc32)[C@H]1C(=O)O)c1ncc(C(C)(C)C)s1] |
| 200 | 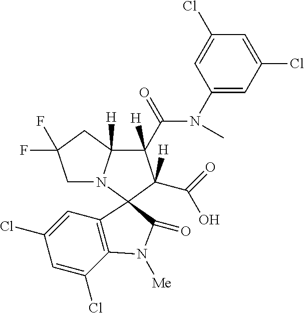 [SMILES: CN(C(=O)[C@H]1[C@H]2CC(F)(F)CN2[C@]2(C(=O)N(C)c3c(Cl)cc(Cl)cc32)[C@H]1C(=O)O)c1cc(Cl)cc(Cl)c1] |
| 201 | 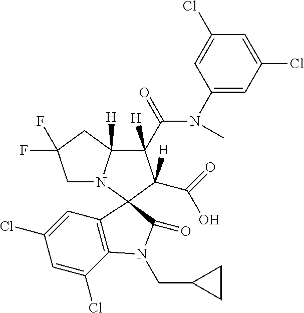 [SMILES: CN(C(=O)[C@H]1[C@H]2CC(F)(F)CN2[C@]2(C(=O)N(CC3CC3)c3c(Cl)cc(Cl)cc32)[C@H]1C(=O)O)c1cc(Cl)cc(Cl)c1] |
| 202 | 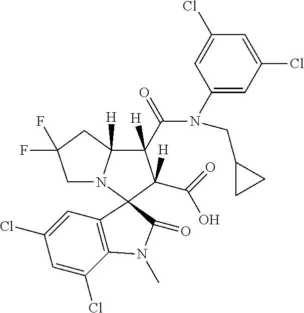 [SMILES: CN1C(=O)[C@]2(c3cc(Cl)cc(Cl)c31)[C@@H](C(=O)O)[C@@H](C(=O)N(CC1CC1)c1cc(Cl)cc(Cl)c1)[C@H]1CC(F)(F)CN12] |
| 203 | 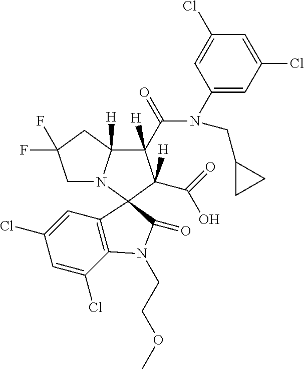 [SMILES: COCCN1C(=O)[C@]2(c3cc(Cl)cc(Cl)c31)[C@@H](C(=O)O)[C@@H](C(=O)N(CC1CC1)c1cc(Cl)cc(Cl)c1)[C@H]1CC(F)(F)CN12] |
| 204 | 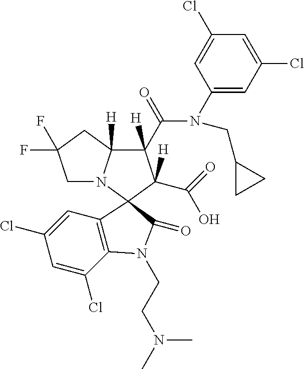 [SMILES: CN(C)CCN1C(=O)[C@]2(c3cc(Cl)cc(Cl)c31)[C@@H](C(=O)O)[C@@H](C(=O)N(CC1CC1)c1cc(Cl)cc(Cl)c1)[C@H]1CC(F)(F)CN12] |
| 205 | 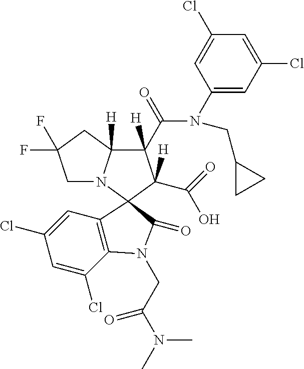 [SMILES: CN(C)C(=O)CN1C(=O)[C@]2(c3cc(Cl)cc(Cl)c31)[C@@H](C(=O)O)[C@@H](C(=O)N(CC1CC1)c1cc(Cl)cc(Cl)c1)[C@H]1CC(F)(F)CN12] |
| 206 | 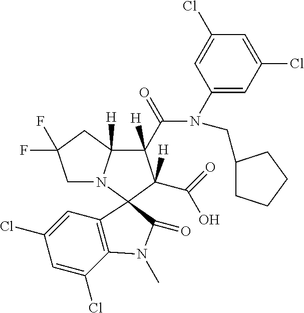 [SMILES: CN1C(=O)[C@]2(c3cc(Cl)cc(Cl)c31)[C@@H](C(=O)O)[C@@H](C(=O)N(CC1CCCC1)c1cc(Cl)cc(Cl)c1)[C@H]1CC(F)(F)CN12] |
| 207 | 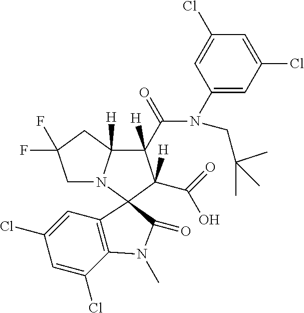 [SMILES: CN1C(=O)[C@]2(c3cc(Cl)cc(Cl)c31)[C@@H](C(=O)O)[C@@H](C(=O)N(CC(C)(C)C)c1cc(Cl)cc(Cl)c1)[C@H]1CC(F)(F)CN12] |
| 208 | 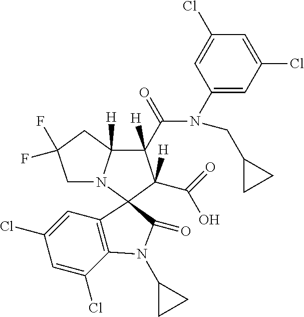 [SMILES: O=C(O)[C@H]1[C@@H](C(=O)N(CC2CC2)c2cc(Cl)cc(Cl)c2)[C@H]2CC(F)(F)CN2[C@]12C(=O)N(C1CC1)c1c(Cl)cc(Cl)cc12] |
| 216.8a | 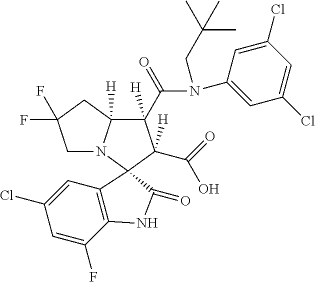 [SMILES: CC(C)(C)CN(C(=O)[C@H]1[C@@H](C(=O)O)[C@]2(C(=O)Nc3c(F)cc(Cl)cc32)N2CC(F)(F)C[C@@H]12)c1cc(Cl)cc(Cl)c1] |
| 216.8b | 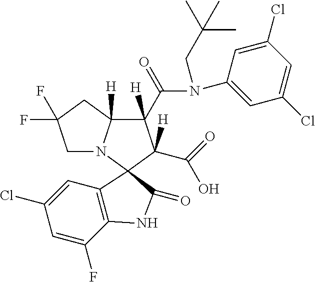 [SMILES: CC(C)(C)CN(C(=O)[C@H]1[C@H]2CC(F)(F)CN2[C@]2(C(=O)Nc3c(F)cc(Cl)cc32)[C@H]1C(=O)O)c1cc(Cl)cc(Cl)c1] |
| 217a | 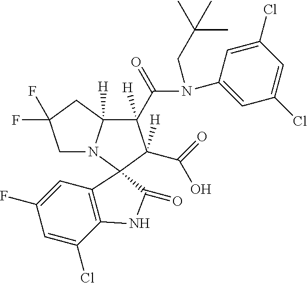 [SMILES: CC(C)(C)CN(C(=O)[C@H]1[C@@H](C(=O)O)[C@]2(C(=O)Nc3c(Cl)cc(F)cc32)N2CC(F)(F)C[C@@H]12)c1cc(Cl)cc(Cl)c1] |
| 217b | 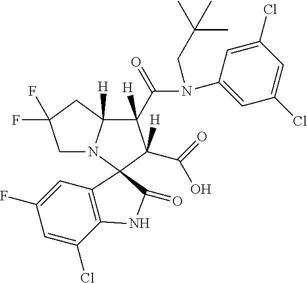 [SMILES: CC(C)(C)CN(C(=O)[C@H]1[C@H]2CC(F)(F)CN2[C@]2(C(=O)Nc3c(Cl)cc(F)cc32)[C@H]1C(=O)O)c1cc(Cl)cc(Cl)c1] |
| 218a | 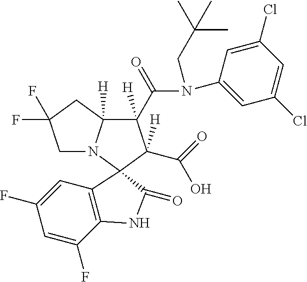 [SMILES: CC(C)(C)CN(C(=O)[C@H]1[C@@H](C(=O)O)[C@]2(C(=O)Nc3c(F)cc(F)cc32)N2CC(F)(F)C[C@@H]12)c1cc(Cl)cc(Cl)c1] |
| 218b | 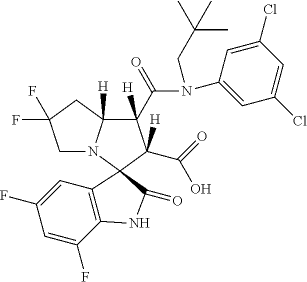 [SMILES: CC(C)(C)CN(C(=O)[C@H]1[C@H]2CC(F)(F)CN2[C@]2(C(=O)Nc3c(F)cc(F)cc32)[C@H]1C(=O)O)c1cc(Cl)cc(Cl)c1] |
| 219 | 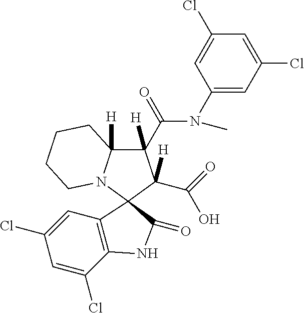 [SMILES: CN(C(=O)[C@H]1[C@H]2CCCCN2[C@]2(C(=O)Nc3c(Cl)cc(Cl)cc32)[C@H]1C(=O)O)c1cc(Cl)cc(Cl)c1] |
| 220.7a 220.7c | 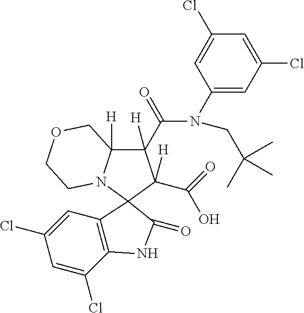 [SMILES: CC(C)(C)CN(C(=O)C1C2COCCN2C2(C(=O)Nc3c(Cl)cc(Cl)cc32)C1C(=O)O)c1cc(Cl)cc(Cl)c1] |
| 225a 225b | 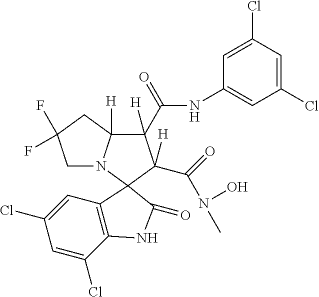 [SMILES: CN(O)C(=O)C1C(C(=O)Nc2cc(Cl)cc(Cl)c2)C2CC(F)(F)CN2C12C(=O)Nc1c(Cl)cc(Cl)cc12] |
| 226a 226b | 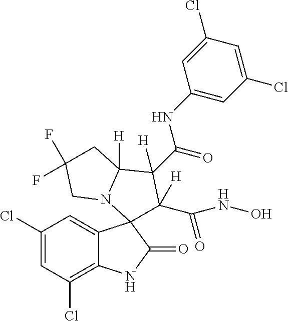 [SMILES: O=C(Nc1cc(Cl)cc(Cl)c1)C1C2CC(F)(F)CN2C2(C(=O)Nc3c(Cl)cc(Cl)cc32)C1C(=O)NO] |
| 227 | 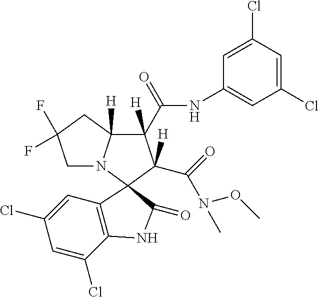 [SMILES: CON(C)C(=O)[C@H]1[C@@H](C(=O)Nc2cc(Cl)cc(Cl)c2)[C@H]2CC(F)(F)CN2[C@]12C(=O)Nc1c(Cl)cc(Cl)cc12] |
| 228 | 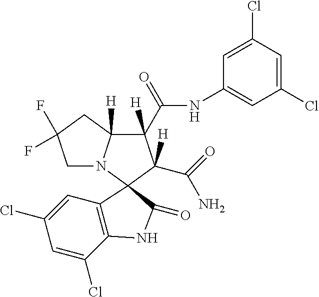 [SMILES: NC(=O)[C@H]1[C@@H](C(=O)Nc2cc(Cl)cc(Cl)c2)[C@H]2CC(F)(F)CN2[C@]12C(=O)Nc1c(Cl)cc(Cl)cc12] |
| 229a 229b | 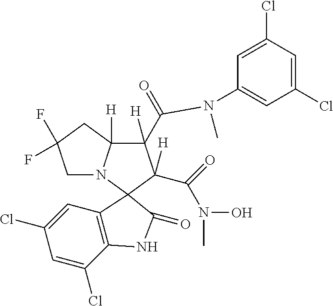 [SMILES: CN(O)C(=O)C1C(C(=O)N(C)c2cc(Cl)cc(Cl)c2)C2CC(F)(F)CN2C12C(=O)Nc1c(Cl)cc(Cl)cc12] |
| 230 | 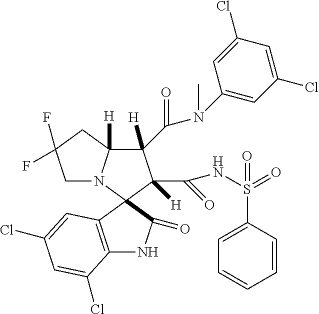 |
| 231 | 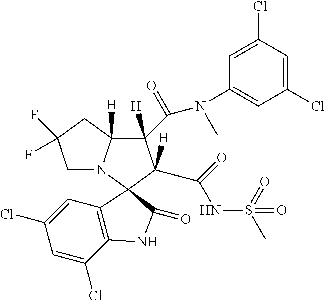 [SMILES: CN(C(=O)[C@H]1[C@H]2CC(F)(F)CN2[C@]2(C(=O)Nc3c(Cl)cc(Cl)cc32)[C@H]1C(=O)NS(C)(=O)=O)c1cc(Cl)cc(Cl)c1] |
| 232a | 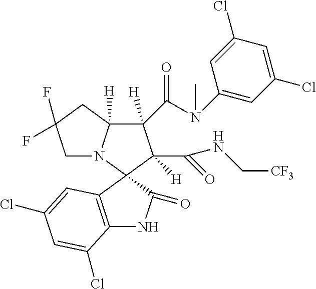 [SMILES: CN(C(=O)[C@H]1[C@@H](C(=O)NCC(F)(F)F)[C@]2(C(=O)Nc3c(Cl)cc(Cl)cc32)N2CC(F)(F)C[C@@H]12)c1cc(Cl)cc(Cl)c1] |
| 232b | 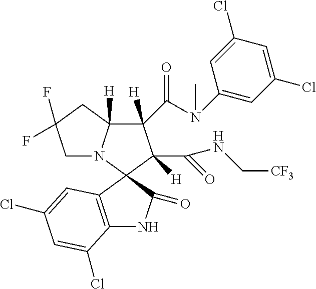 [SMILES: CN(C(=O)[C@H]1[C@H]2CC(F)(F)CN2[C@]2(C(=O)Nc3c(Cl)cc(Cl)cc32)[C@H]1C(=O)NCC(F)(F)F)c1cc(Cl)cc(Cl)c1] |
| 233 | 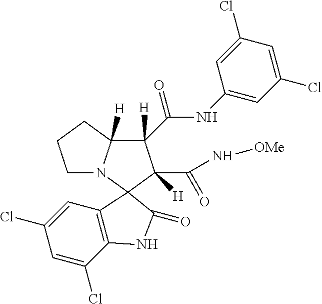 [SMILES: CONC(=O)[C@H]1[C@@H](C(=O)Nc2cc(Cl)cc(Cl)c2)[C@H]2CCCN2C12C(=O)Nc1c(Cl)cc(Cl)cc12] |
| 234a 234b | 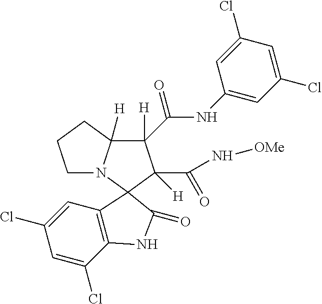 [SMILES: CONC(=O)C1C(C(=O)Nc2cc(Cl)cc(Cl)c2)C2CCCN2C12C(=O)Nc1c(Cl)cc(Cl)cc12] |
| 235a 235b | 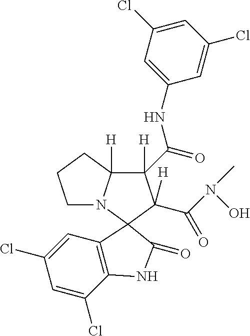 [SMILES: CN(O)C(=O)C1C(C(=O)Nc2cc(Cl)cc(Cl)c2)C2CCCN2C12C(=O)Nc1c(Cl)cc(Cl)cc12] |
| 236 | 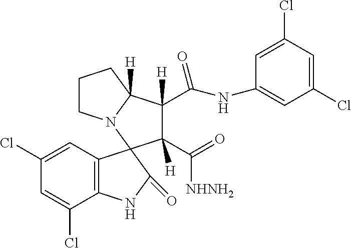 [SMILES: NNC(=O)[C@H]1[C@@H](C(=O)Nc2cc(Cl)cc(Cl)c2)[C@H]2CCCN2C12C(=O)Nc1c(Cl)cc(Cl)cc12] |
| 237 | 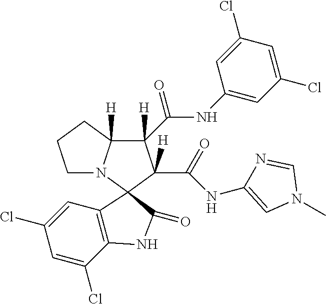 [SMILES: Cn1cnc(NC(=O)[C@H]2[C@@H](C(=O)Nc3cc(Cl)cc(Cl)c3)[C@H]3CCCN3[C@]23C(=O)Nc2c(Cl)cc(Cl)cc23)c1] |
| 238 | 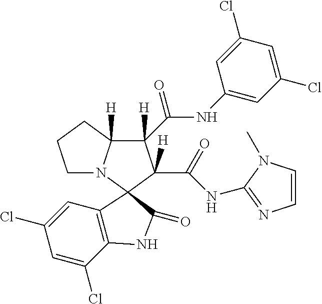 [SMILES: Cn1ccnc1NC(=O)[C@H]1[C@@H](C(=O)Nc2cc(Cl)cc(Cl)c2)[C@H]2CCCN2[C@]12C(=O)Nc1c(Cl)cc(Cl)cc12] |
| 239 | 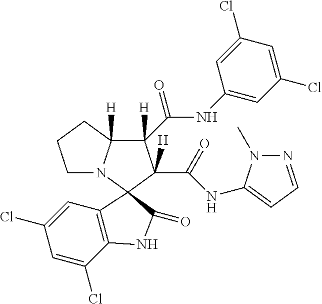 [SMILES: Cn1nccc1NC(=O)[C@H]1[C@@H](C(=O)Nc2cc(Cl)cc(Cl)c2)[C@H]2CCCN2[C@]12C(=O)Nc1c(Cl)cc(Cl)cc12] |
| 240 | 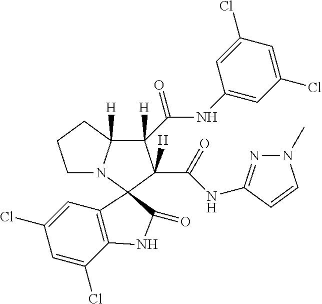 [SMILES: Cn1ccc(NC(=O)[C@H]2[C@@H](C(=O)Nc3cc(Cl)cc(Cl)c3)[C@H]3CCCN3[C@]23C(=O)Nc2c(Cl)cc(Cl)cc23)n1] |
| 241 | 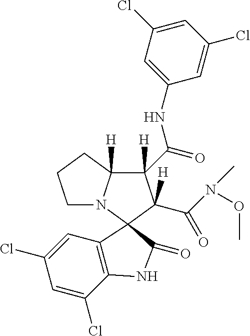 [SMILES: CON(C)C(=O)[C@H]1[C@@H](C(=O)Nc2cc(Cl)cc(Cl)c2)[C@H]2CCCN2[C@]12C(=O)Nc1c(Cl)cc(Cl)cc12] |
| 242 | 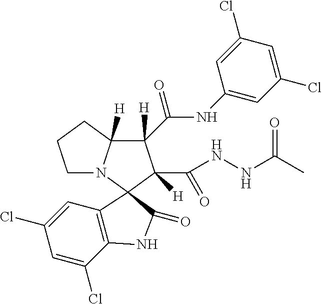 [SMILES: CC(=O)NNC(=O)[C@H]1[C@@H](C(=O)Nc2cc(Cl)cc(Cl)c2)[C@H]2CCCN2[C@]12C(=O)Nc1c(Cl)cc(Cl)cc12] |
| 243 | 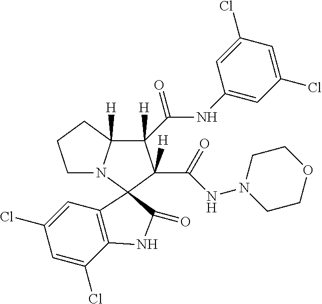 [SMILES: O=C(Nc1cc(Cl)cc(Cl)c1)[C@H]1[C@H]2CCCN2[C@]2(C(=O)Nc3c(Cl)cc(Cl)cc32)[C@H]1C(=O)NN1CCOCC1] |
| 244 | 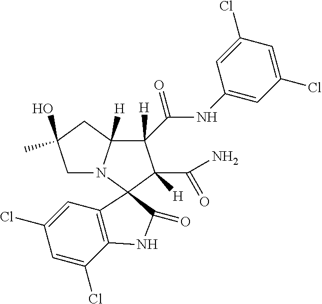 [SMILES: C[C@]1(O)C[C@@H]2[C@H](C(=O)Nc3cc(Cl)cc(Cl)c3)[C@H](C(N)=O)[C@@]3(C(=O)Nc4c(Cl)cc(Cl)cc43)N2C1] |
| 245 | 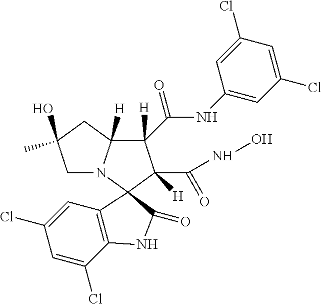 [SMILES: C[C@]1(O)C[C@@H]2[C@H](C(=O)Nc3cc(Cl)cc(Cl)c3)[C@H](C(=O)NO)[C@@]3(C(=O)Nc4c(Cl)cc(Cl)cc43)N2C1] |
| 246 | 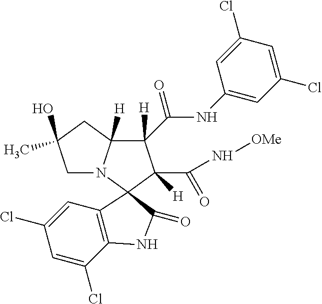 [SMILES: CONC(=O)[C@H]1[C@@H](C(=O)Nc2cc(Cl)cc(Cl)c2)[C@H]2C[C@](C)(O)CN2[C@]12C(=O)Nc1c(Cl)cc(Cl)cc12] |
| 247 | 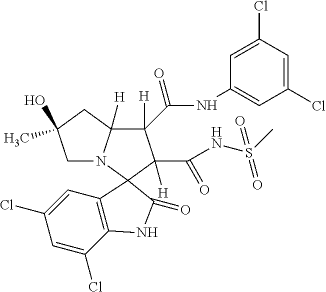 [SMILES: C[C@]1(O)CC2C(C(=O)Nc3cc(Cl)cc(Cl)c3)C(C(=O)NS(C)(=O)=O)C3(C(=O)Nc4c(Cl)cc(Cl)cc43)N2C1] |
| 248 | 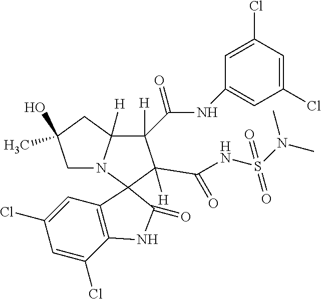 [SMILES: CN(C)S(=O)(=O)NC(=O)C1C(C(=O)Nc2cc(Cl)cc(Cl)c2)C2C[C@](C)(O)CN2C12C(=O)Nc1c(Cl)cc(Cl)cc12] |
| 249a | 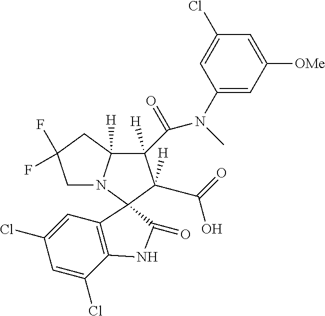 [SMILES: COc1cc(Cl)cc(N(C)C(=O)[C@H]2[C@@H](C(=O)O)[C@]3(C(=O)Nc4c(Cl)cc(Cl)cc43)N3CC(F)(F)C[C@@H]23)c1] |
| 249b | 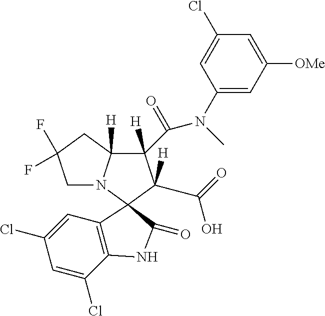 [SMILES: COc1cc(Cl)cc(N(C)C(=O)[C@H]2[C@H]3CC(F)(F)CN3[C@]3(C(=O)Nc4c(Cl)cc(Cl)cc43)[C@H]2C(=O)O)c1] |
| 250.3a | 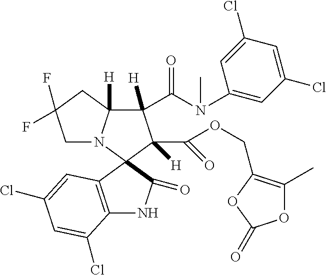 |
| 250.3b | 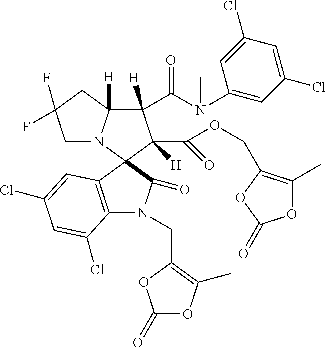 |
| 251a | 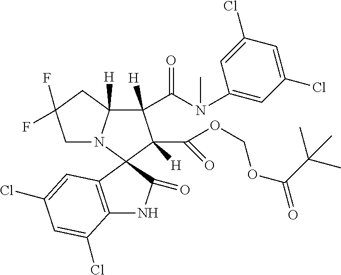 [SMILES: CN(C(=O)[C@H]1[C@H]2CC(F)(F)CN2[C@]2(C(=O)Nc3c(Cl)cc(Cl)cc32)[C@H]1C(=O)OCOC(=O)C(C)(C)C)c1cc(Cl)cc(Cl)c1] |
| 251c | 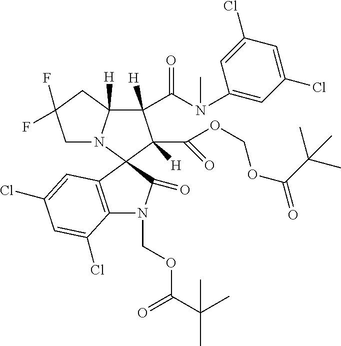 [SMILES: CN(C(=O)[C@H]1[C@H]2CC(F)(F)CN2[C@]2(C(=O)N(COC(=O)C(C)(C)C)c3c(Cl)cc(Cl)cc32)[C@H]1C(=O)OCOC(=O)C(C)(C)C)c1cc(Cl)cc(Cl)c1] |
| 252 | 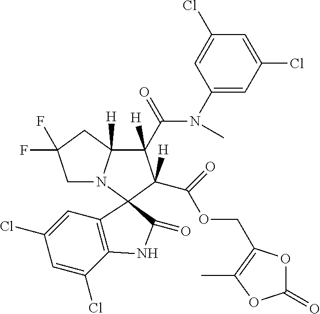 [SMILES: Cc1oc(=O)oc1COC(=O)[C@H]1[C@@H](C(=O)N(C)c2cc(Cl)cc(Cl)c2)[C@H]2CC(F)(F)CN2[C@]12C(=O)Nc1c(Cl)cc(Cl)cc12] |
| 253 | 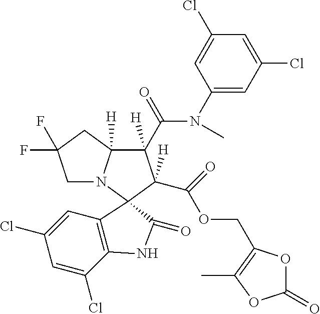 [SMILES: Cc1oc(=O)oc1COC(=O)[C@@H]1[C@H](C(=O)N(C)c2cc(Cl)cc(Cl)c2)[C@@H]2CC(F)(F)CN2[C@@]12C(=O)Nc1c(Cl)cc(Cl)cc12] |
| 254a | 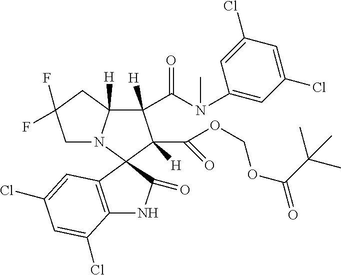 [SMILES: CN(C(=O)[C@H]1[C@H]2CC(F)(F)CN2[C@]2(C(=O)Nc3c(Cl)cc(Cl)cc32)[C@H]1C(=O)OCOC(=O)C(C)(C)C)c1cc(Cl)cc(Cl)c1] |
| 254b | 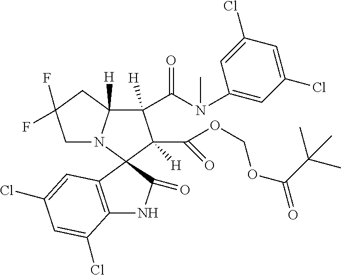 [SMILES: CN(C(=O)[C@@H]1[C@H]2CC(F)(F)CN2[C@]2(C(=O)Nc3c(Cl)cc(Cl)cc32)[C@@H]1C(=O)OCOC(=O)C(C)(C)C)c1cc(Cl)cc(Cl)c1] |
| 255a | 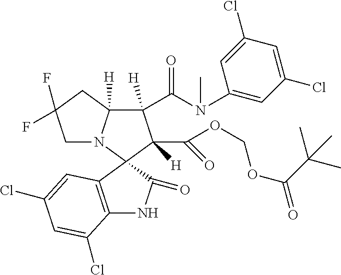 [SMILES: CN(C(=O)[C@@H]1[C@@H]2CC(F)(F)CN2[C@@]2(C(=O)Nc3c(Cl)cc(Cl)cc32)[C@H]1C(=O)OCOC(=O)C(C)(C)C)c1cc(Cl)cc(Cl)c1] |
| 255b | 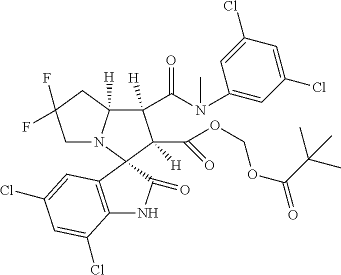 [SMILES: CN(C(=O)[C@H]1[C@@H](C(=O)OCOC(=O)C(C)(C)C)[C@]2(C(=O)Nc3c(Cl)cc(Cl)cc32)N2CC(F)(F)C[C@@H]12)c1cc(Cl)cc(Cl)c1] |
| 256a | 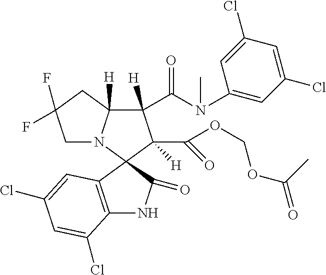 [SMILES: CC(=O)OCOC(=O)[C@@H]1[C@@H](C(=O)N(C)c2cc(Cl)cc(Cl)c2)[C@H]2CC(F)(F)CN2[C@]12C(=O)Nc1c(Cl)cc(Cl)cc12] |
| 256b | 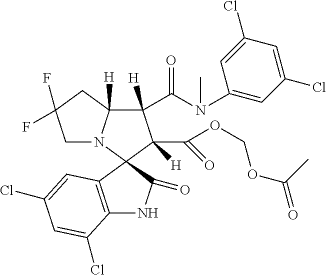 [SMILES: CC(=O)OCOC(=O)[C@H]1[C@@H](C(=O)N(C)c2cc(Cl)cc(Cl)c2)[C@H]2CC(F)(F)CN2[C@]12C(=O)Nc1c(Cl)cc(Cl)cc12] |
| 257 | 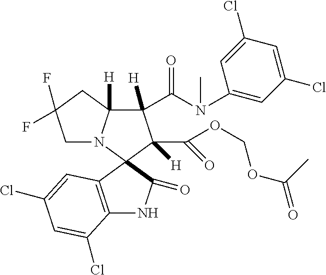 |
| 258 | 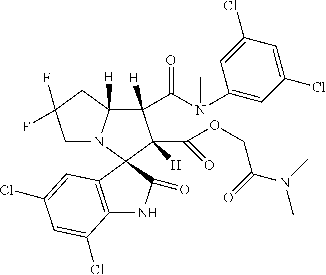 [SMILES: CN(C)C(=O)COC(=O)[C@H]1[C@@H](C(=O)N(C)c2cc(Cl)cc(Cl)c2)[C@H]2CC(F)(F)CN2[C@]12C(=O)Nc1c(Cl)cc(Cl)cc12] |
| 259 | 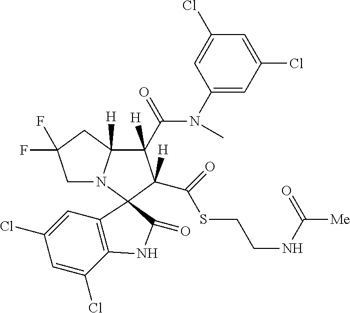 [SMILES: CC(=O)NCCSC(=O)[C@H]1[C@@H](C(=O)N(C)c2cc(Cl)cc(Cl)c2)[C@H]2CC(F)(F)CN2[C@]12C(=O)Nc1c(Cl)cc(Cl)cc12] |
| 260a | 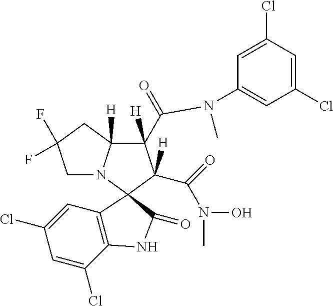 [SMILES: CN(O)C(=O)[C@H]1[C@@H](C(=O)N(C)c2cc(Cl)cc(Cl)c2)[C@H]2CC(F)(F)CN2[C@]12C(=O)Nc1c(Cl)cc(Cl)cc12] |
| 260b | 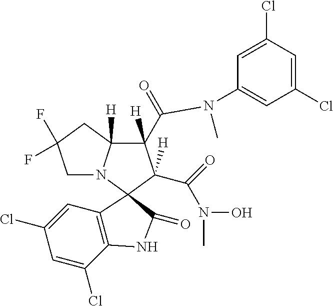 [SMILES: CN(O)C(=O)[C@@H]1[C@@H](C(=O)N(C)c2cc(Cl)cc(Cl)c2)[C@H]2CC(F)(F)CN2[C@]12C(=O)Nc1c(Cl)cc(Cl)cc12] |
| 261 | 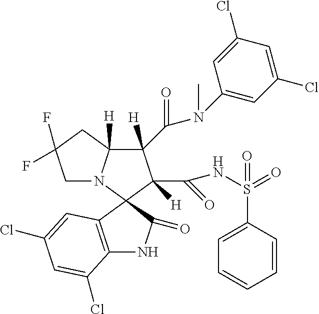 [SMILES: CN(C(=O)[C@H]1[C@H]2CC(F)(F)CN2[C@]2(C(=O)Nc3c(Cl)cc(Cl)cc32)[C@H]1C(=O)NS(=O)(=O)c1ccccc1)c1cc(Cl)cc(Cl)c1] |
| 265a | 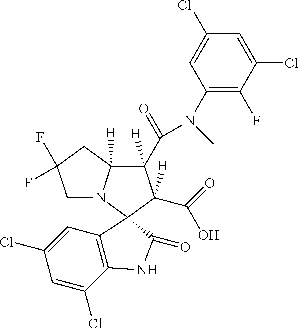 [SMILES: CN(C(=O)[C@H]1[C@@H](C(=O)O)[C@]2(C(=O)Nc3c(Cl)cc(Cl)cc32)N2CC(F)(F)C[C@@H]12)c1cc(Cl)cc(Cl)c1F] |
| 265b | 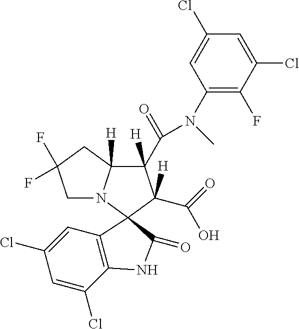 [SMILES: CN(C(=O)[C@H]1[C@H]2CC(F)(F)CN2[C@]2(C(=O)Nc3c(Cl)cc(Cl)cc32)[C@H]1C(=O)O)c1cc(Cl)cc(Cl)c1F] |
| 266 | 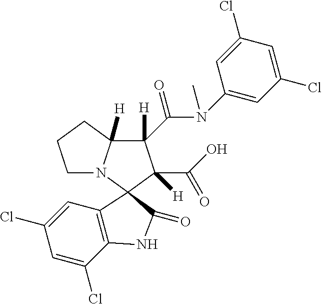 [SMILES: CN(C(=O)[C@H]1[C@H]2CCCN2[C@]2(C(=O)Nc3c(Cl)cc(Cl)cc32)[C@H]1C(=O)O)c1cc(Cl)cc(Cl)c1] |
| 267 | 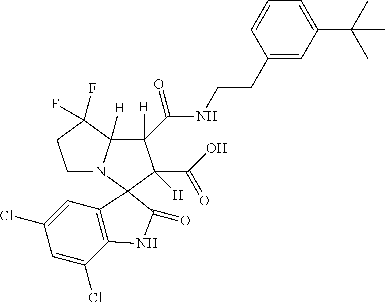 [SMILES: CC(C)(C)c1cccc(CCNC(=O)C2C3N(CCC3(F)F)C3(C(=O)Nc4c(Cl)cc(Cl)cc43)C2C(=O)O)c1] |
| 268a 268b | 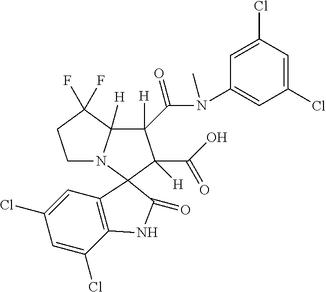 [SMILES: CN(C(=O)C1C2N(CCC2(F)F)C2(C(=O)Nc3c(Cl)cc(Cl)cc32)C1C(=O)O)c1cc(Cl)cc(Cl)c1] |
| 269a | 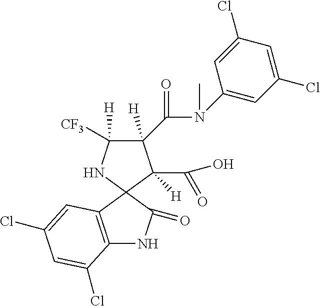 [SMILES: CN(C(=O)[C@@H]1[C@H](C(F)(F)F)NC2(C(=O)Nc3c(Cl)cc(Cl)cc32)[C@@H]1C(=O)O)c1cc(Cl)cc(Cl)c1] |
| 269b | 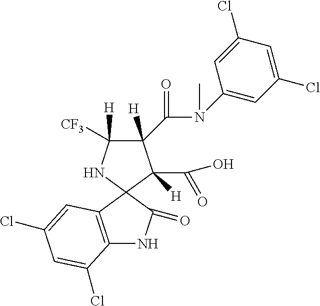 [SMILES: CN(C(=O)[C@H]1[C@@H](C(F)(F)F)NC2(C(=O)Nc3c(Cl)cc(Cl)cc32)[C@H]1C(=O)O)c1cc(Cl)cc(Cl)c1] |
| 270a | 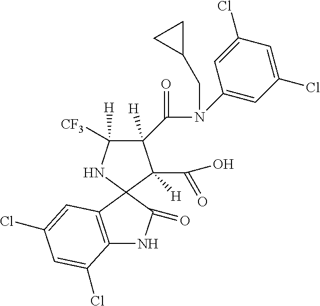 [SMILES: O=C(O)[C@@H]1[C@H](C(=O)N(CC2CC2)c2cc(Cl)cc(Cl)c2)[C@H](C(F)(F)F)NC12C(=O)Nc1c(Cl)cc(Cl)cc12] |
| 270b | 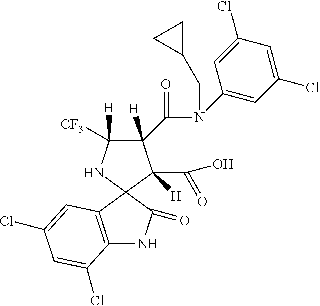 [SMILES: O=C(O)[C@H]1[C@@H](C(=O)N(CC2CC2)c2cc(Cl)cc(Cl)c2)[C@@H](C(F)(F)F)NC12C(=O)Nc1c(Cl)cc(Cl)cc12] |
| 271 | 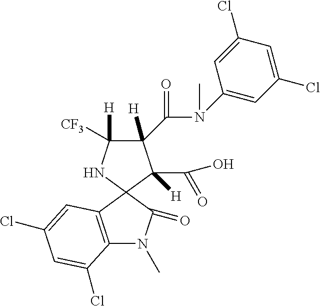 [SMILES: CN(C(=O)[C@H]1[C@@H](C(F)(F)F)NC2(C(=O)N(C)c3c(Cl)cc(Cl)cc32)[C@H]1C(=O)O)c1cc(Cl)cc(Cl)c1] |
| 272 | 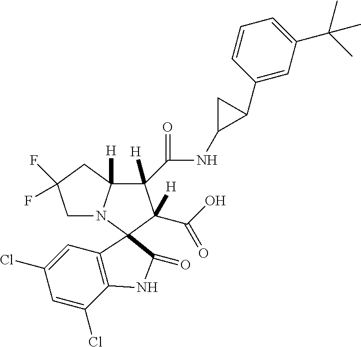 |
| 273a 273b | 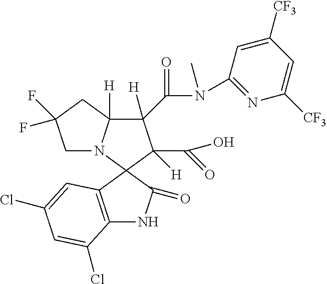 [SMILES: CN(C(=O)C1C2CC(F)(F)CN2C2(C(=O)Nc3c(Cl)cc(Cl)cc32)C1C(=O)O)c1cc(C(F)(F)F)cc(C(F)(F)F)n1] |
| 274.6a | 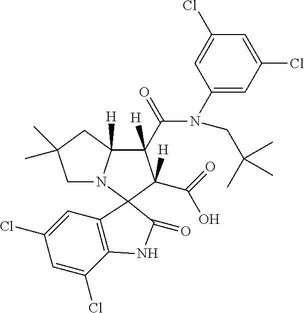 [SMILES: CC(C)(C)CN(C(=O)[C@H]1[C@H]2CC(C)(C)CN2C2(C(=O)Nc3c(Cl)cc(Cl)cc32)[C@H]1C(=O)O)c1cc(Cl)cc(Cl)c1] |
| 274.6b | 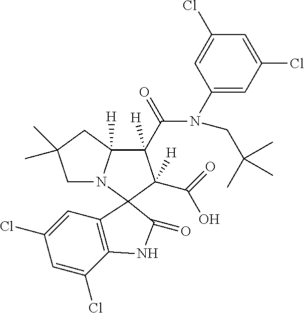 [SMILES: CC(C)(C)CN(C(=O)[C@H]1[C@@H](C(=O)O)C2(C(=O)Nc3c(Cl)cc(Cl)cc32)N2CC(C)(C)C[C@@H]12)c1cc(Cl)cc(Cl)c1] |
| 275.7a | 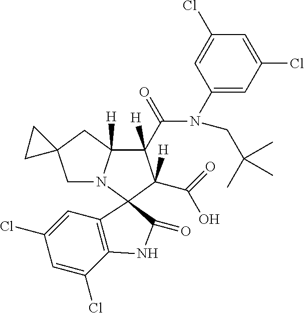 [SMILES: CC(C)(C)CN(C(=O)[C@H]1[C@H]2CC3(CC3)CN2[C@]2(C(=O)Nc3c(Cl)cc(Cl)cc32)[C@H]1C(=O)O)c1cc(Cl)cc(Cl)c1] |
| 275.7b | 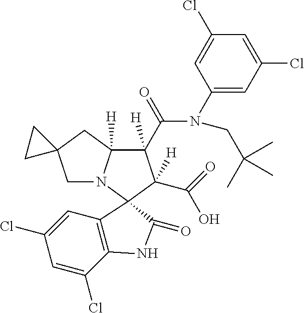 [SMILES: CC(C)(C)CN(C(=O)[C@H]1[C@@H](C(=O)O)[C@]2(C(=O)Nc3c(Cl)cc(Cl)cc32)N2CC3(CC3)C[C@@H]12)c1cc(Cl)cc(Cl)c1] |
| 280 | 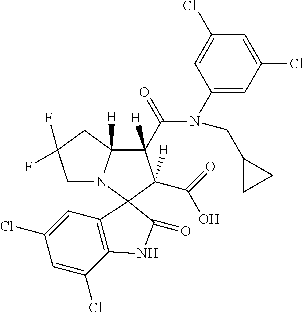 [SMILES: O=C(O)[C@@H]1[C@@H](C(=O)N(CC2CC2)c2cc(Cl)cc(Cl)c2)[C@H]2CC(F)(F)CN2C12C(=O)Nc1c(Cl)cc(Cl)cc12] |
| 281 | 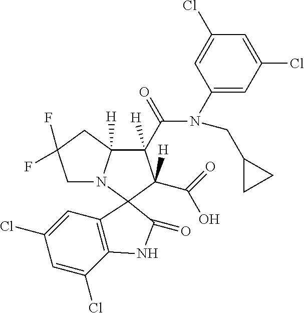 [SMILES: O=C(O)[C@H]1[C@H](C(=O)N(CC2CC2)c2cc(Cl)cc(Cl)c2)[C@@H]2CC(F)(F)CN2C12C(=O)Nc1c(Cl)cc(Cl)cc12] |
| 282 | 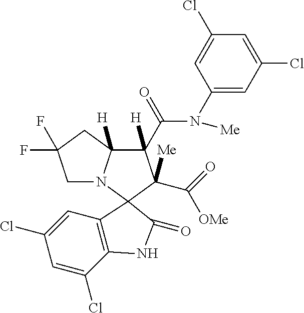 [SMILES: COC(=O)[C@@]1(C)[C@@H](C(=O)N(C)c2cc(Cl)cc(Cl)c2)[C@H]2CC(F)(F)CN2C12C(=O)Nc1c(Cl)cc(Cl)cc12] |
| 284 | 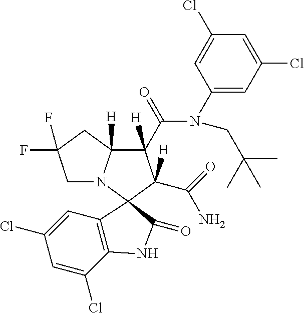 [SMILES: CC(C)(C)CN(C(=O)[C@H]1[C@H]2CC(F)(F)CN2[C@]2(C(=O)Nc3c(Cl)cc(Cl)cc32)[C@H]1C(N)=O)c1cc(Cl)cc(Cl)c1] |
| 285 | 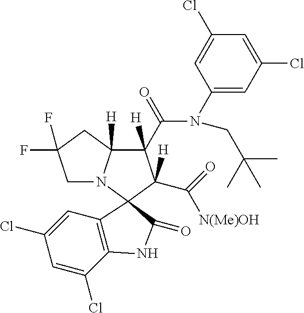 |
| 286a 286b | 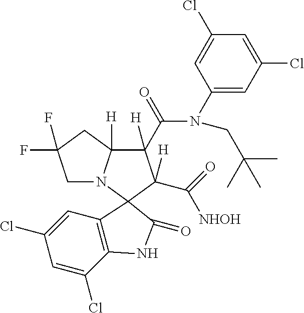 |
| 287 | 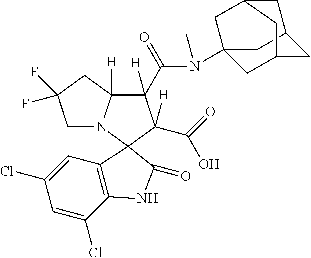 [SMILES: CN(C(=O)C1C2CC(F)(F)CN2C2(C(=O)Nc3c(Cl)cc(Cl)cc32)C1C(=O)O)C12CC3CC(CC(C3)C1)C2] |
| 288a | 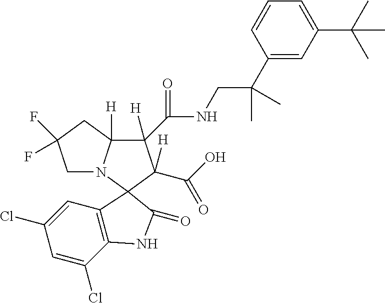 [SMILES: CC(C)(C)c1cccc(C(C)(C)CNC(=O)C2C3CC(F)(F)CN3C3(C(=O)Nc4c(Cl)cc(Cl)cc43)C2C(=O)O)c1] |
| 288b | 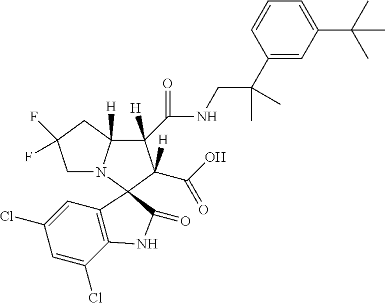 [SMILES: CC(C)(C)c1cccc(C(C)(C)CNC(=O)[C@H]2[C@H]3CC(F)(F)CN3[C@]3(C(=O)Nc4c(Cl)cc(Cl)cc43)[C@H]2C(=O)O)c1] |
| 289 | 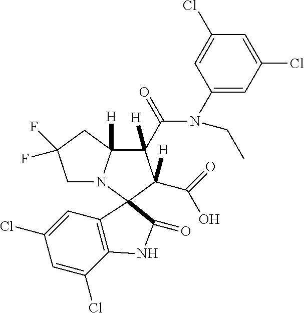 |
| 290 | 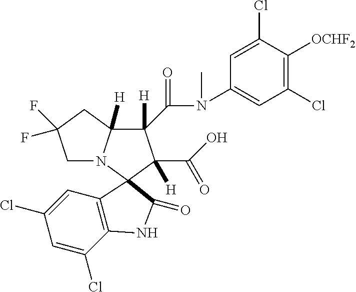 |
| 291 | 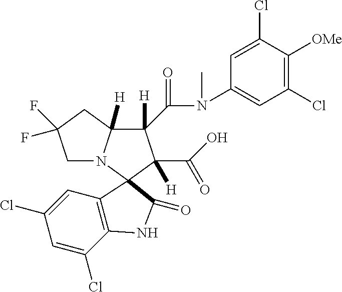 |
| 292 | 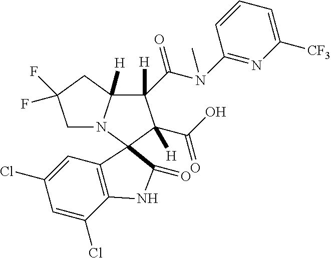 |
| 293 | 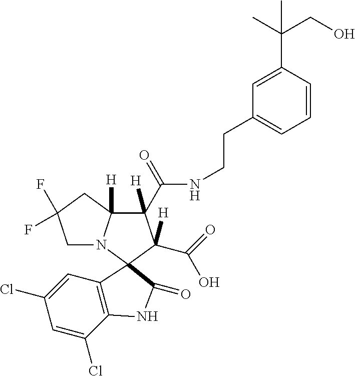 |
| 294 | 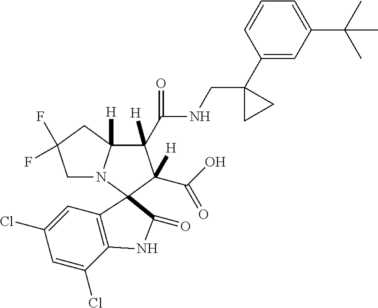 |

37. A pharmaceutical composition comprising a compound of claim 1 and a pharmaceutically acceptable carrier or excipient.

38. A method of treating a disease or disorder mediated by cystic fibrosis transmembrane conductance regulator (CFTR) in a subject in need thereof, comprising administering to the subject a therapeutically effective amount of a compound of claim 1.

39. (canceled)

40. A method for treating cystic fibrosis in a subject in need thereof, comprising the step of administering to the subject a therapeutically effective amount of a compound according claim 1.

41. The method of claim 40, further comprising the step of administering to the subject a therapeutically effective amount of a compound which is a CFTR modulator, a mucolytic or an antibiotic.
